(19) United States
(12) Patent Application Publication
(10) Pub. No.: US 2025/0288625 A1
(43) Pub. Date: Sep. 18, 2025

(54) COMPOSITIONS AND METHODS FOR TREATING ANTIBIOTIC RESISTANCE

(71) Applicant: Seres Therapeutics, Inc., Cambridge, MA (US)

(72) Inventors: Christopher Burton FORD, Swampscott, MA (US); Jessica BRYANT, Cambridge, MA (US); Edward J. O'BRIEN, Arlington, MA (US)

(73) Assignee: Seres Therapeutics, Inc., Cambridge, MA (US)

(21) Appl. No.: 19/005,746

(22) Filed: Dec. 30, 2024

Related U.S. Application Data
(62) Division of application No. 16/760,757, filed on Apr. 30, 2020, now Pat. No. 12,214,002.
(60) Provisional application No. 62/578,698, filed on Oct. 30, 2017.
(60) Provisional application No. 62/741,346, filed on Oct. 4, 2018.

(51) Int. Cl. A61K 35/742; A61P 31/04

(52) U.S. Cl. CPC A61K 35/742; A61P 31/04

(57) ABSTRACT

The present invention relates to compositions and methods for reducing the carriage of antibiotic resistance genes.

### CROSS REFERENCE TO RELATED APPLICATIONS

This application is a division of U.S. patent application Ser. No. 16/760,757, 371 (c) date of Apr. 30, 2020, which is the National Stage of International Application No. PCT/US2018/058279, filed Oct. 30, 2018, which claims the benefit of U.S. Provisional Application No. 62/578,698, filed Oct. 30, 2017, and of U.S. Provisional Application No. 62/741,346, filed Oct. 4, 2018, each of which is incorporated herein by reference in its entirety.

### REFERENCE TO A SEQUENCE LISTING SUBMITTED ELECTRONICALLY

The content of the electronically submitted sequence listing (Name: 4268_0450003_SequenceListing_ST26.xml; Size: 965,092 bytes; and Date of Creation: Jan. 9, 2025) filed with this application is herein incorporated by reference it its entirety.

### FIELD OF THE INVENTION

The invention relates to preventing and treating antibiotic resistance.

### BACKGROUND

According to the World Health Organization (WHO), bacterial “[a]ntibiotic resistance is one of the biggest threats to global health, food security, and development today” (who.int/mediacentre/factsheets/antibiotic-resistance/en). Antibiotic resistance can occur naturally in bacteria but is greatly exacerbated by increasing use and misuse of antibiotics. Adding to the problem is the diversity of antibiotic resistance mechanisms; a variety of disparate genes can confer resistance, and unrelated bacteria can horizontally transmit antibiotic resistance genes. The WHO has also stated “[w]hile there are some new antibiotics in development, none of them are expected to be effective against the most dangerous forms of antibiotic-resistant bacteria (ibid).”

Accordingly, it is imperative that new methods of reducing antibiotic resistance be developed and made available.

### SUMMARY

The invention relates to the discovery that bacterial compositions can be used to reduce the abundance of antibiotic resistance genes in the gastrointestinal (GI) microbiome of a subject.

The invention provides compositions including a therapeutically effective amount of a microbiome composition comprising spore-former bacteria for use in reducing the abundance of at least one antibiotic-resistance gene in the microbiome of a subject.

The invention also provides compositions including a therapeutically effective amount of a microbiome composition comprising Firmicutes derived from one or more healthy human subjects for use in treating a subject at risk for or diagnosed with an undesirable level or population of drug-resistant bacteria.

The invention further provides ROAR compositions for use reducing the abundance of antibiotic-resistant bacteria in the microbiome of a subject.

The invention additionally provides ROAR compositions for use in disrupting a transmission cycle of antibiotic resistance genes, by administration to at least two individuals with a high likelihood of direct or indirect contact, including optionally contact with a bodily fluid or waste.

In addition, the invention provides methods of reducing the abundance of at least one antibiotic-resistance gene in the microbiome of a subject, the methods including administering a therapeutically effective amount of a microbiome composition containing spore-former bacteria.

The invention also provides methods of treating a subject at risk for or diagnosed with an undesirable level or population of drug-resistant bacteria, the methods including administering a therapeutically effective amount of a microbiome composition containing Firmicutes derived from one or more healthy human subjects.

The invention further provides methods of reducing the abundance of antibiotic-resistant bacteria in the microbiome of a subject, the methods including administering a therapeutically effective amount of a ROAR composition.

The invention additional provides methods of disrupting a transmission cycle of antibiotic resistance genes, the methods including administering a ROAR composition to at least two individuals with a high likelihood of direct or indirect contact including contact with a bodily fluid or waste.

Various embodiments of any of the methods noted above are as set forth below.

In some embodiments, the antibiotic-resistance gene(s) is selected from those listed in Table 1.

In some embodiments, the abundance of at least 2, 3, 4, 5, 6, 7, 8, 9, 10, or more antibiotic resistance genes of Table 1 is reduced.

In some embodiments, the drug or antibiotic is from a drug class listed in Table 2.

In some embodiments, the methods further include, or the compositions for use further result in, reducing the abundance of 1, 2, 3, 4, 5, 6, 7, 8, 9, 10, 11, 12, 13, 14, 15, 16, 17, 18, 19, 20, 21, 22, 23, 24, 25, or more antibiotic resistant genes or drug classes.

In some embodiments, the composition includes one or more species listed in Table 3. In some embodiments, the species listed in Table 3 is not also listed in Table 4. The composition may be administered by a method described above or elsewhere herein.

In some embodiments, the composition includes 2, 3, 4, 5, 6, 7, 8, 9, 10, 11, 12, 13, 14, 15, 16, 17, 18, 19, 20, or more species listed in Table 3. In some embodiments, the species listed in Table 3 is not also listed in Table 4. The composition may be administered by a method described above or elsewhere herein.

In some embodiments, the species include a 16S rDNA, V4, and/or V6 sequence listed in Table 5, or a sequence having at least 95% identity thereto. The species may be administered by a method described above or elsewhere herein.

In some embodiments, the composition does not include one or more species of Table 4. In some embodiments, the species listed in Table 4 that is not included is not listed Table 3. The composition may be administered by a method described above or elsewhere herein.

In some embodiments, the species does not include a 16S rDNA, V4, and/or V6 sequence listed in Table 6, or a sequence having at least 95% identity thereto. The species may be administered by a method described above or elsewhere herein.

In some embodiments the bacteria in the composition are in the form of spores.

In some embodiments, the bacteria in the composition are cultured.

In some embodiments, the composition includes Firmicutes and one or more species of Bacteroides.

In some embodiments, the subject has a reduced abundance of one, two, or three bacteria selected from the group consisting of quinolone resistant, beta-lactam-resistant, and carbapenem-resistant bacteria.

In some embodiments, the subject has been unresponsive to antibiotic treatment.

In some embodiments, the subject has or is at risk of developing C. difficile infection or disease, or a colitis wherein, in some embodiments, the colitis is optionally Crohn's disease or ulcerative colitis (e.g., mild to moderate ulcerative colitis). In some embodiments, the subject does not have (or is not risk of developing) C. difficile infection or disease, or a colitis (such as Crohn's disease or ulcerative colitis, e.g., mild to moderate ulcerative colitis).

The invention also provides compositions including bacterial species associated with a decrease in the abundance of at least one antibiotic resistance gene in a subject.

In some embodiments, the antibiotic-resistance gene(s) is selected from those listed in Table 1.

In some embodiments, the abundance of at least 2, 3, 4, 5, 6, 7, 8, 9, 10, or more antibiotic resistance genes of Table 1 is decreased.

In some embodiments, the antibiotic is from a class listed in Table 2.

In some embodiments, the composition includes one or more species listed in Table 3. In some embodiments, the species listed in Table 3 is not also listed in Table 4.

In some embodiments, the composition includes 2, 3, 4, 5, 6, 7, 8, 9, 10, 11, 12, 13, 14, 15, 16, 17, 18, 19, 20, or more species listed in Table 3. In some embodiments, the species listed in Table 3 is not also listed in Table 4.

In some embodiments, the species include a 16S rDNA, V4, and/or V6 sequence listed in Table 5, or a sequence having at least 95% identity thereto.

In some embodiments, the composition does not include one or more species of Table 4. In some embodiments, the species of Table 4 that is not included is not listed in Table 3.

In some embodiments, the species do not include a 16S rDNA, V4, and/or V6 sequence listed in Table 6, or a sequence having at least 95% identity thereto.

In some embodiments, the composition includes one or more species of Firmicutes.

In some embodiments, the composition includes one or more species of Bacteroides.

In some embodiments, the bacteria in the composition are in the form of spores.

In some embodiments, the bacteria in the composition are obtained from a spore fraction derived from feces of a healthy subject.

In some embodiments, the composition includes one or more species of Firmicutes, and optionally one or more species of Bacteroides, as the sole bacterial species in the composition. In some embodiments, the bacteria in the composition are cultured.

The invention further provides compositions including bacteria for use in decreasing the abundance of at least one antibiotic resistance gene in a subject to whom the composition is administered.

In some embodiments, the bacteria in the composition are obtained from a spore fraction derived from feces of a healthy subject.

In some embodiments, the bacteria are selected for being associated with a decrease in the abundance of one or more antibiotic resistance genes in a subject.

In some embodiments, the antibiotic resistance gene(s) is selected from Table 1.

In some embodiments, the composition includes at least one species selected from Table 3.

In some embodiments, the composition does not include a species of Table 4.

The invention also provides methods for identifying a bacterial composition useful for decreasing the abundance of at least one antibiotic resistance gene in a subject to whom the composition is administered, the methods including screening the composition for the presence of one or more bacterial species of Table 3, wherein detection of one or more species of Table 3 in the bacterial composition indicates the identification of a composition that can be used to decrease the abundance of at least one antibiotic resistance gene. In some embodiments, the species of Table 3 is not also listed in Table 4.

In some embodiments, the screening includes detection of one or more 16S rDNA, V4, and/or V6 sequences of Table 5 in the composition, or a sequence having at least 95% identity thereto.

In some embodiments, the methods further include screening for the presence of one or more bacterial species of Table 4. In some embodiments, the species screened for from Table 4 is also not listed in Table 3.

In some embodiments, the screening includes detection of one or more 16S rDNA, V4, and/or V6 sequences of Table 6 in the composition, or a sequence having at least 95% identity thereto. The invention also includes methods for screening a potential donor of feces for use in therapeutic methods, the methods including testing a feces sample from the potential donor for the presence of a bacterial species from Table 3 or Table 4, wherein detection of one or more species from Table 3 indicates that the potential donor may proceed to become a donor, while detection of one or more species from Table 4 indicates that the potential donor should not become a donor. In some embodiments, the species from Table 3 tested for is not listed in Table 4. In some embodiments, the species from Table 4 tested for is not listed in Table 3.

The invention further includes methods for determining whether a subject could benefit from treatment with a ROAR composition, the methods including determining whether a sample from the subject includes one or more species of bacteria from Table 4, wherein detection of one or more species from Table 4 indicates that the subject may benefit from treatment with a ROAR composition. In some embodiments, the species from Table 3 tested for is not listed in Table 4. In some embodiments, the species from Table 4 tested for is not listed in Table 3.

The invention further provides compositions (e.g., the compositions described above and elsewhere herein), wherein the bacteria in the compositions demonstrate the ability to decrease the abundance of one or more antibiotic resistance gene(s) in an animal model of antibiotic resistance. In some embodiments, the animal model is a mouse treated with one or more antibiotics and then colonized with one or more bacteria harboring one or more antibiotic resistance genes. In some embodiments, the mice are colonized with vancomycin resistant bacteria or vancomycin and carbapenem resistant bacteria.

The invention also includes use of the compositions described herein for the purposes specified in the methods described herein, as well as the use of these compositions for the preparation of medicaments for these uses.

The entire disclosure of each patent document and scientific article referred to herein, and those patent documents and scientific articles cited thereby, is expressly incorporated by reference herein for all purposes.

Additional features and advantages of the invention are more particularly described below.

### BRIEF DESCRIPTION OF THE DRAWINGS

FIG. 1 is a graph depicting the results of an experiment detecting the reduction of genes associated with fluoroquinolone resistance in human subjects 7, 28, and 56 days after dosing with a human commensal spore composition. White=subjects who received placebo, medium gray=all subjects treated with the spore composition, and dark gray=results from the subset of subjects with a significant engraftment signal.

FIG. 2 is a graph depicting the results of an experiment detecting the reduction of genes associated with beta-lactam antibiotic resistance in human subjects 7, 28, and 56 days after dosing with a human commensal spore composition. White=subjects who received placebo, medium gray=all subjects treated with the spore composition, dark gray=results from the subset of subjects with a significant engraftment signal.

FIG. 3 is a graph showing the abundance of antibiotic resistance genes per time point (x-axis), faceted by cohort (A: placebo, B: spore prepared from the stool of healthy, screened humans, weekly, D: vancomycin, spore preparation weekly, and C: vancomycin, spore preparation daily.

FIG. 4 is a graph showing that in subjects with rapid, early engraftment with spore preparation in Arm D (Vancomycin, spore preparation Weekly), antibiotic resistance genes decreased more rapidly as compared to subjects who did not exhibit rapid, early engraftment. Visit 4 (day 8) is immediately following cessation of antibiotics, and Visit 4 (day 11) is the first spore preparation time point.

### DETAILED DESCRIPTION

Antibiotic resistance (referred to herein as “AbxR”) is a growing global public health issue. Resistance to all classes of antibiotics has been reported in all parts of the world (2014, Antimicrobial Resistance, Global Report on Surveillance, Summary, WHO) and development of conventional antibiotics has been relatively slow compared to the demand. For example, no new classes of antibiotics have been commercialized since the discovery of the lipopeptide antibiotic daptomycin in the 1980s. Furthermore, antibiotic stewardship programs that limit antibiotic use do not immediately reverse the trend of increasing resistance.

Applicants have discovered that treating a subject with a composition comprising bacteria, for example, spore-former bacteria derived from a healthy human, can reduce the abundance of AbxR genes (collectively, the “gut resistome”) in a human subject. Such compositions are referred to herein as reduction of antibiotic resistance compositions (ROAR compositions).

Furthermore, such treatment can reduce the abundance of AbxR genes associated with resistance to multiple classes of antibiotics, e.g., β-lactams (including carbapenems), quinolones, glycopeptides (e.g., vancomycin), and tetracyclines. In addition, it was found that non-pathogenic commensals can harbor antibiotic resistance genes, and treatment results in a decrease in the abundance of antibiotic resistance genes in non-pathogenic commensal organisms. Without committing to any particular theory, these data support a method of decreasing the presence of antibiotic resistance genes by, at least in part, a mechanism of decreasing horizontal transfer of antibiotic resistance genes from non-pathogenic commensal organisms to pathogenic bacteria. Such a mechanism could be related at least in part to decreasing the abundance of bacteria harboring antibiotic resistance genes by out-competing these bacteria for nutrients; decreasing the abundance of bacteria harboring antibiotic resistance genes by inducing a response in the host that limits the growth of potential pathogens harboring AbxR genes; decreasing the abundance of bacteria harboring antibiotic resistance genes by introducing bacteria that are directly antagonistic to potential pathogens harboring AbxR genes; reducing opportunistic pathogens, and/or decreasing the potential for the spread of antibiotic resistance genes between antibiotic resistant and susceptible organisms. In some embodiments, the decrease in the presence of antibiotic resistance genes occurs by reducing colonization in the host of microbes that harbor AbxR genes.

In some embodiments, a ROAR composition is used to break or prevent an antibiotic resistance transmission cycle. In such methods, a ROAR composition can be administered to a subject entering an environment in which there is a risk of an antibiotic resistance cycle or an environment in which the existence of an antibiotic resistance cycle has been identified, e.g., an environment in which antibiotic resistance has been identified as spreading or in which there is a risk of such spreading. Examples of such environments include hospitals, intensive care units, nursing homes, rehabilitation centers, group homes, and the like. In some embodiments, the dosing in such an environment is chronic. In some embodiments, a subject is treated to prevent or reduce colonization by one or more AbxR genes. In a subject that harbors one or more AbxR genes, a composition can be administered, thereby reducing the risk of AbxR infection in that subject and reducing the risk that subject will transmit AbxR genes after treatment of C. difficile disease, as such subjects may be susceptible to infection with or harbor antibiotic resistant bacteria. In some embodiments, a composition is administered to workers in environments in which there is a particular risk associated with spreading AbxR genes.

In some environments, “selective decontamination” of patients has been recommended, for example, in hospitals (particularly intensive care units) and nursing homes (for example, see Silvestri and van Saene, “Selective decontamination of the digestive tract: an update of the evidence,” HSR Proc. Intensive Care Cardiovasc. Anesth. 4:21-29, 2012; Oostdijk et al., “Selective decontamination in European intensive care patients,” Intensive Care Med. 38:533-538, 2012). A ROAR composition can be a useful alternative or supplement to such selective decontamination, adding to the armamentarium of methods available to decrease the presence of antibiotic resistance genes in these and other settings.

### ROAR Compositions

ROAR compositions generally comprise Firmicutes. In some embodiments, the Firmicutes are capable of forming spores (spore-formers). Bacteria in a composition can be in the form of spores, mixed vegetative and spore forms, or vegetative form. In some cases, a composition consists of Firmicutes. In some embodiments, a ROAR composition comprises one or more species of Bacteroidetes. In some embodiments, a ROAR composition consists of at least one species from each of Firmicutes and Bacteroidetes. Compositions comprising or consisting of Firmicutes (or Firmicutes and Bacteroidetes) as the sole bacterial species of the compositions are not naturally occurring.

As a non-limiting example, a ROAR composition can be prepared from bacteria that are isolated from stool, e.g., human stool. In general, the stool is from a healthy animal (e.g., a human) of the same species as the animal to be treated. Bacteria can be prepared, for example, in a method using an amount of ethanol suitable to kill any non-spore form bacteria present in the stool. An example of such a preparation can be found in, for example, WO 2014/121302.

In some embodiments, a ROAR composition comprises or consists of selected bacterial species associated with a decrease in the abundance of antibiotic resistance genes in a subject (see, e.g., Table 3, below). Such compositions are referred to herein as “designed Abx compositions.” The number of species (e.g., from Table 3) in a designed composition can be, for example, 1, 2, 3, 4, 5, 6, 7, 8, 9, 10, 11, 12, 13, 14, 15, 16, 17, 18, 19, 20, or more. In some embodiments the number of species is at least one and less than or equal to 50, for example, less than 45, less than 40, less than 35, less than 30, less than 25, less than 20, less than 15, less than 10, or less than 5. In some embodiments, the species selected from Table 3 do not include species that are also listed in Table 4.

Individual species in a designed ROAR composition may be cultured separately and then combined to create the composition. In some embodiments, a ROAR composition is produced by co-culturing all or subsets of the composition and then combining the components that were not co-cultured to create the composition. In some embodiments, a ROAR composition is produced by culturing a spore fraction derived from feces of a healthy human. The resulting culture can be used as a ROAR composition.

In some embodiments, the species selected for inclusion in a ROAR composition are associated with a decrease in the abundance of a particular class of antibiotic resistance genes, e.g., genes associated with resistance to quinolones, beta-lactams, and/or carbapenems (also see Table 2). In specific examples of, e.g., the compositions described above, bacterial species in a ROAR composition (e.g., designed Abx compositions) are selected from Table 2, and optionally may be identified by analysis of 16S rDNA sequences (or fragments thereof, e.g., a fragment comprising the 16S rDNA V4 and/or V6 regions; also see below and Table 5 for exemplary sequences).

In some embodiments, a species useful in a ROAR composition is a species having a full-length 16S rDNA with at least 95% sequence identity (“identity”) (e.g., at least 96%, 97%, 98%, or 99%, or 100%) to the 16S rDNA of a reference species, e.g., a species identified in Table 3 (see, e.g., Table 5 for sequence information). In some embodiments, a species useful in a ROAR composition is a species having a full-length 16S rDNA with at least 97% sequence identity (“identity”) to the 16S rDNA of a reference species, e.g., a species in Table 3 (see, e.g., Table 5 for sequence information). In some embodiments, a useful species has a V4 and/or V6 region 16S rDNA sequence having at least 95% identity (e.g., at least 96%, 97%, 98%, or 99%, or 100%) to a V4 and/or V6 region of 16S rDNA sequence of a reference species, e.g., a species identified in Table 3 (see, e.g., Table 5 for sequence information). In some embodiments, a useful species has a V4 and/or V6 region 16S rDNA sequence having at least 97% identity to a V4 and/or V6 region of 16S rDNA sequence of a reference species, e.g., a species identified in Table 3 (see, e.g., Table 5 for sequence information). In some embodiments, a useful species has a genomic sequence having at least 95% identity (e.g., at least 96%, 97%, 98%, or 99%, or 100%) to the full-length genomic DNA of a reference species, e.g., a species identified in Table 3. In some embodiments a useful species has a genomic sequence having at least 97% identity to the full-length genomic sequence of a reference species, e.g., a species identified in Table 3. In the event that a sequence is not provided herein, e.g., a 16S rDNA, V4, or V6 sequence, methods are well known in the art for identifying such sequences. Table 5 provides non-limiting examples of full-length 16S rDNA sequences that can be used as reference sequences. In general, identity or percent identity with a reference species means identity or percent identity with at least one 16S rDNA sequence (or fragment; e.g., V4 and/or V6) found in an organism.

In some cases, strains of bacterial species useful in the invention, e.g., species disclosed herein, can be obtained from a public biological resource center such as the ATCC (atcc.org), the DSMZ (dsmz.de), or the Riken BioResource Center (en.brc.riken.jp). 16s rDNA sequences useful for identifying species or other aspects of the invention can be obtained from public databases, e.g., the Human Microbiome Project (HMP) web site or GenBank.

Methods of determining sequence identity are known in the art and examples are provided infra.

In some embodiments, species excluded from ROAR compositions are species that are positively correlated with the presence of a particular class of antibiotic resistance genes (see, e.g., Table 4). These species can be identified using methods such as those described above (also see Table 6 for corresponding sequence information).

It is to be understood that “consisting of” in these examples refers to bacteria types that are present in a composition. A bacterial formulation “consisting of” listed species may thus contain additional non-bacterial materials such as one or more excipients (including, for example, one or more capsules), an aqueous or non-aqueous medium (e.g., glycerol, polyethylene glycol, cocoa butter, water, and/or a buffer), and/or one or more prebiotics or small molecule drugs.

### Species/Naming Information

Names and classification of bacteria are subject to changes that may not be reflected in the literature. For convenience, alternate names for some bacterial species may be provided herein but are not intended to be a comprehensive set of alternative names. In some embodiments, species are identified by sequence identity of all or a portion (e.g., V4 or V6 regions) of a 16S rDNA sequence, e.g., at least 90%, 93% 95%, 96%, 97%, 98%, 99%, or 100% identity.

### Determination of Identity

Clades, operational taxonomic units (OTUs), species, and strains are, in some embodiments, identified by their 16S rDNA sequence. The relatedness of clades, OTUs, species, and strains can be determined by the percent identity between clades, OTUs, species, or strains. Percent identity between a reference and query sequence can be determined using methods known in the art. Non-limiting examples of methods for such determinations are provided below. As used herein, the relatedness between two nucleotide sequences is described by the parameter “identity.”

In one embodiment, the degree of sequence identity between a query sequence and a reference sequence is determined by (1) aligning the two sequences by any suitable alignment program using the default scoring matrix and default gap penalty, (2) identifying the number of exact matches, where an exact match is where the alignment program has identified an identical nucleotide in the two aligned sequences on a given position in the alignment, and (3) dividing the number of exact matches with the length of the reference sequence.

In another embodiment, the degree of sequence identity between a query sequence and a reference sequence is determined by (1) aligning the two sequences by any suitable alignment program using the default scoring matrix and default gap penalty, (2) identifying the number of exact matches, where an exact match is where the alignment program has identified an identical nucleotide in the two aligned sequences on a given position in the alignment, and (3) dividing the number of exact matches with the length of the longest of the two sequences.

In another embodiment, the degree of sequence identity between the query sequence and the reference sequence is determined by (1) aligning the two sequences by any suitable alignment program using the default scoring matrix and default gap penalty, (2) identifying the number of exact matches, where an exact match is where the alignment program has identified an identical amino acid or nucleotide in the two aligned sequences on a given position in the alignment, and (3) dividing the number of exact matches with the “alignment length,” where the alignment length is the length of the entire alignment including gaps and overhanging parts of the sequences.

Sequence identity comparisons are carried out, generally, with the aid of a sequence comparison program. These commercially or publicly available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalizing the insertion of gaps, gap extensions, and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.

In general, the default values of the alignment program are used for sequence comparisons. Suitable computer programs useful for determining identity include, for example, BLAST (blast.ncbi.nlm.nih.gov).

In an embodiment of the present invention, the alignment program optimizes the alignment over the full-length of selected sequences, e.g., full-length, V4, or V6 16S rDNA sequence. For example, the global alignment program is based on the Needleman-Wunsch algorithm (Needleman and Wunsch, J. Mol. Biol. 48:443-453, 1970). Non-limiting examples of such programs are EMBOSS Needle and EMBOSS Stretcher programs, available at ebi.ac.uk/Tools/psa/.

In one embodiment, the sequences are aligned by a global alignment program and the sequence identity is calculated by identifying the number of exact matches identified by the program divided by the “alignment length,” where the alignment length is the length of the entire alignment including gaps and overhanging parts of the sequences. In a further embodiment, the global alignment program uses the Needleman-Wunsch algorithm and the sequence identity is calculated by identifying the number of exact matches identified by the program divided by the “alignment length,” where the alignment length is the length of the entire alignment including gaps and overhanging parts of the sequences.

In yet a further embodiment, the global alignment program is selected from the group consisting of EMBOSS Needle and EMBOSS stretcher and the sequence identity is calculated by identifying the number of exact matches identified by the program divided by the “alignment length,” where the alignment length is the length of the entire alignment including gaps and overhanging parts of the sequences.

Once the software has produced an alignment, it is possible to calculate percent (%) similarity and percent sequence identity.

### Methods of Testing Compositions for AbxR Activity or the Presence of AbxR Genes

A ROAR composition can be tested for the ability to reduce the abundance of antibiotic resistance genes in the GI tract of an animal using in vitro and/or in vivo models known in the art (for example, see Schjørring and Krogfelt, Int. J. Microbiol. 2011:312956, 2011; Marra, “Animal models in drug development for MRSA,” Meth. Mol. Biol. 1085:333-345, 2014). Such models can be adapted for use in testing candidate ROAR compositions. Additional examples of suitable models are described in the Examples. In addition, a ROAR composition can be tested for the ability to reduce the abundance of AbxR genes in human subjects enrolled in clinical trials.

### Subjects

In some embodiments, a subject suitable for treatment with a composition disclosed herein (a ROAR composition) is an animal, e.g., a mammal such as a human, dog, cat, bovine, ovine, coprine, equine, or other farm, domestic, or zoo animal at risk for, or diagnosed with, having an undesirable level of AbxR microorganisms, e.g., in at least a portion of the gastrointestinal (GI) tract. An undesirable level of AbxR organisms can be identified as a level significantly greater than that in a healthy population, for example, in the context of treating humans, the healthy population documented in the Human Microbiome Project database. Such organisms can be detected by, for example, culturing a stool or other gastrointestinal sample from the subject and using known methods of testing for the presence of antibiotic resistance. The methods include, for example, dilution methods (broth and agar dilution methods), disk-diffusion methods, E-test, automated methods, mechanism-specific tests such as beta-lactamase detection test and chromogenic cephalosporin tests, and genotypic methods such as PCR and DNA hybridization methods. Such methods also include the identification and quantification of antibiotic resistance associated genes by whole metagenomics shotgun (WMS) sequencing. In some embodiments, an undesirable level of AbxR organisms or genes can be identified using molecular methods that identify the presence or absence of genes, gene cassettes, or genetic markers associated with the phenotype of AbxR. Some examples of such molecular methods include but are not limited to DNA sequencing, transcriptional profiling, and methods that utilize a molecular probe.

In some embodiments, a subject suitable for treatment with a ROAR composition is or has been unresponsive to treatment with one or more antibiotics and the presence of an antibiotic resistant microbial population is inferred.

In some embodiments, a subject suitable for treatment with a ROAR composition has or is at risk of developing C. difficile infection or disease. In some embodiments, a subject suitable for treatment with a ROAR composition has or is at risk of developing a colitis (e.g., Crohn's disease or ulcerative colitis (e.g., mild to moderate ulcerative colitis)).

### Advantages

Use of conventional antibiotics, in addition to the potential for the existence or development of antibiotic resistance by a subject's microbiome, can cause serious side effects including, for example, GI effects such as dysbiosis, overgrowth of pathogens, nervous system damage, and allergic reactions. A ROAR composition typically comprises commensal bacteria and has limited side effects, e.g., is well-tolerated by subjects receiving the treatment. A ROAR composition can be administered repeatedly over time, as needed, and may reduce the risk of developing antibiotic resistance to a large group of antibiotics, e.g., by reducing the prevalence of antibiotic resistance genes in a treated individual. In general, a ROAR composition can modulate transmission cycles in which bacteria transmit antibiotic resistance genes. This feature is useful in, for example, health care settings in which the use of the compositions decreases the risk of fragile patients being exposed to or developing antibiotic resistance, both individually and as a patient population.

In some embodiments, an additional advantage of an ROAR composition that administration of the composition may improve the integrity of the epithelial and mucosal barriers, for example, by reducing the amount of local inflammation, and reducing the risk of translocation and infection by GI local microbes.

### Definitions

“Augmentation” of a type of bacterium, e.g., a species, is an effect of treatment with a composition of the invention that is characterized by post-treatment detection of an increased abundance of a species not present in the composition by a nonparametric test of abundance.

“Engraftment” of a type of bacterium, e.g., a species, is an effect of treatment with a composition of the invention that is characterized by post-treatment detection of a species from the administered composition, which is not detected in the treated subject pretreatment. Methods of detection are known in the art. In one example, the method is PCR detection of a 16S rDNA sequence using standard parameters for PCR.

“Operational taxonomic unit,” “OTU” (or plural, “OTUs”) refers to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence is the 16S sequence or a portion of the 16S sequence, such as a variable region, e.g., a V4 region. In other embodiments, the entire genomes of two entities are sequenced and compared. In another embodiment, select regions such as multilocus sequence tags (MLST), specific genes, or sets of genes may be genetically compared. In 16S embodiments, OTUs that share ≥97% average nucleotide identity across an entire 16S or a variable region of a 16S sequence are considered the same OTU (see e.g., Claesson et al., Nucleic Acids Res. 38:e200, 2010; Konstantinidis et al., Philos. Trans. R. Soc. Lond. B. Biol. Sci. 361:1929-1940, 2006). In embodiments involving the complete genome, MLSTs, specific genes, or sets of genes, OTUs that share ≥95% average nucleotide identity are considered the same OTU (see e.g. Achtman and Wagner, Nat. Rev. Microbiol. 6:431-440, 2008). OTUs can be distinguished, in some embodiments, by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence. As used herein, the terms “species” and “OTU” are used interchangeably unless otherwise distinguished by context.

A “therapeutically effective amount” of a composition described herein can vary according to factors such as the disease state, age, sex, and weight of the individual, and the ability of the composition to elicit a desired response in the individual, e.g., amelioration of at least one disorder parameter, or amelioration of at least one symptom of the disorder (and optionally, the effect of any additional agents being administered). A therapeutically effective amount is also one in which any toxic or detrimental effects of the composition are outweighed by the therapeutically beneficial effects. A composition as described herein is generally administered in a therapeutically effective amount.

### Formulations

ROAR compositions (e.g., designed Abx compositions) described herein can be prepared and administered using methods known in the art. In general, compositions are formulated for oral, colonoscopic, or nasogastric delivery although any appropriate method can be used.

A ROAR formulation can contain one or more pharmaceutical excipients suitable for the preparation or delivery of such formulations. In some embodiments, the formulation is a liquid formulation. In some embodiments, a formulation comprising a ROAR composition can comprise one or more of surfactants, adjuvants, buffers, antioxidants, tonicity adjusters, thickeners, viscosity modifiers, and the like.

In some embodiments, treatment includes administering a ROAR composition in a formulation that includes a pharmaceutically acceptable carrier. In some embodiments, the excipient includes a capsule or other format suitable for providing the ROAR composition as an oral dosage form. When an excipient serves as a diluent, it can be a solid, semi-solid, or liquid material, which acts as a vehicle, carrier, or medium for the active ingredient. Thus, the formulations can be in the form of tablets, pills, powders, lozenges, sachets, cachets, elixirs, suspensions, emulsions, solutions, syrups, soft or hard capsules, suppositories, or packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, polyethylene glycol, glycerol, and methyl cellulose. The compositions can be formulated so as to provide quick, sustained, or delayed release of the active ingredient after administration to the patient by employing procedures known in the art.

A ROAR composition can be formulated in a unit dosage form. In general, a dosage comprises about 10e2 to 10e9 viable colony forming units (cfu). In some embodiments, the ROAR compositions comprises or is composed of spore-former bacteria in spore form. In such cases, the dosage may be determined as “spore CFU” or “sCFU”, which quantify the number of viable spores by germination and growth, typically on plates.

The term “unit dosage forms” refers to physically discrete units suitable as unitary dosages for human subjects and/or other mammals, each unit containing a predetermined quantity of active material calculated to produce a desired therapeutic effect, in association with a suitable pharmaceutical excipient. A dosage may be administered in multiple delivery vehicles, e.g., multiple pills or capsules.

The amount and frequency of administering a ROAR composition to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest one or more symptoms of the disease or its complications. Effective doses will depend on the disease or condition being treated, as well as by the judgment of the attending clinician, depending upon factors such as the severity of the disease, the age, weight, and general condition of the patient, and the like.

The dosage can refer, for example, to the total number of cfus of each individual species or strains, or can refer to the total number of microorganisms in the dose. It is understood in the art that determining the number of organisms in a dosage is not exact and can depend on the method used to determine the number of organisms present. For example, the number of spores in a composition may be determined using a dipicolinic acid assay. In some cases, the number of organisms may be determined using a culture assay. When spores are present, the ability of assays relying on culture methods can depend on efficient germination of spores. Quantitative nucleic acid-based methods can depend on whether the nucleic acids from non-viable microorganisms are sufficiently reduced or eliminated. Effective doses can be extrapolated from dose-response curves derived from in vitro or animal model test systems.

### Screening Methods

The invention also provides methods for identifying bacterial compositions that can be used to decrease the abundance of antibiotic resistance genes in subjects. In these methods, candidate compositions are analyzed for the presence of one or more bacterial species of Table 3. Detection of one or more (e.g., 2, 3, 4, 5, 6, 7, 8, 9, 10, 11, 12, 13, 14, 15, 16, 17, 18, 19, 20, or more) of these species indicates that the composition may be useful in decreasing the abundance of antibiotic resistance genes. Additional testing can be done to further screen candidate compositions. For example, candidate compositions can further be analyzed for the presence of one or more species of Table 4. In some embodiments, it may be determined that it is preferable to avoid use of compositions identified as including one or more (e.g., 2, 3, 4, 5, 6, 7, 8, 9, 10, 11, 12, 13, 14, 15, 16, 17, 18, 19, 20, or more) species of Table 4.

In other screening methods of the invention, potential donors of feces for use in therapeutic methods (e.g., therapeutic methods of the present invention) are screened for their suitability to serve as donors. In these methods, a fecal sample of a potential donor is screened for the presence of one or more bacterial species associated with decreased antibiotic resistance genes (see, e.g., Table 3; see Table 5 for sequences) and/or one or more bacterial species correlated with the presence of antibiotic resistance genes (see, e.g., Table 4; see Table 6 for sequences). Detection of the former may indicate that the potential donor is suitable to proceed to serve as a donor, while detection of the latter may indicate that the potential donor should not proceed, as can be determined by those of skill in the art.

Additional screening methods include those used to determine whether a subject may benefit from treatment with a ROAR composition, as described herein. In these methods, a sample from the subject (e.g., a fecal sample) is tested for the presence of one or more bacterial species correlated with the presence of antibiotic resistance genes (see, e.g., Tables 4 and 6). Detection of one or more of these species can indicate that the subject may benefit from treatment according to the therapeutic methods of the invention, as can be determined by those of skill in the art.

Detection of species in these methods of the invention can be carried out using methods that are known in the art (see, e.g., above and elsewhere herein). For example, methods including, for example, qPCR, whole metagenomics shotgun sequencing (WMS; see, e.g., below), and/or analysis of 16S rDNA sequences or a portion thereof (e.g., the V4 and/or V6 regions; see, e.g., Table 5) can be performed. Sequences from Table 5 can be detected with respect to the identification of negative correlates (i.e., reduction in antibiotic resistance genes). Sequences from Table 6 can be detected with respect to the identification of positive correlates (i.e., increased abundance of antibiotic resistance genes).

### EQUIVALENTS

All technical features can be individually combined in all possible combinations of such features.

The invention may be embodied in other specific forms without departing from the spirit or essential characteristics thereof. The foregoing embodiments are therefore to be considered in all respects illustrative rather than limiting on the invention described herein.

### Examples

The following non-limiting examples further illustrate embodiments of the inventions described herein.

### Example 1: Human Studies-Clostridium difficile

Human subjects at risk for recurrent Clostridium difficile infection (rCDI) were treated with enriched and purified spores prepared from the stool of healthy, screened humans in a randomized, double-blind, placebo-controlled Phase 2 trial (n=89; randomized 2:1; 36 sites). The spores were administered orally in a single dose (of 4 capsules). Dosing was at 10e8 spore equivalents. The GI microbiomes of these subjects, as well as subjects in a Phase 1b open label dose ranging study, were profiled using whole metagenomics shotgun sequencing (WMS). Stool samples were collected before treatment with the spores (after treatment with antibiotics) and after receiving spores or placebo, as applicable.

DNA was extracted from stool samples and used to generate WMS data with the Illumina sequencing platform. WMS data was screened for the presence and abundance of genetic markers of proteins known to confer antibiotics resistance, as catalogued in the Comprehensive Antibiotic Resistance Database (CARD v.1.1.8) (card.mcmaster.ca). Genetic markers associated with these proteins were constructed using ShortBRED (Kaminski et al., PLOS Comput Biol 11 (12):e1004557), an informatics tool designed to identify proteins of interest at high specificity within WMS data (Table 1). For each class of antibiotics, the abundance of genetic markers associated with resistance to that class summed to generate normalized-gene abundance values (referred to herein as drug class marker-abundance or DCMA).

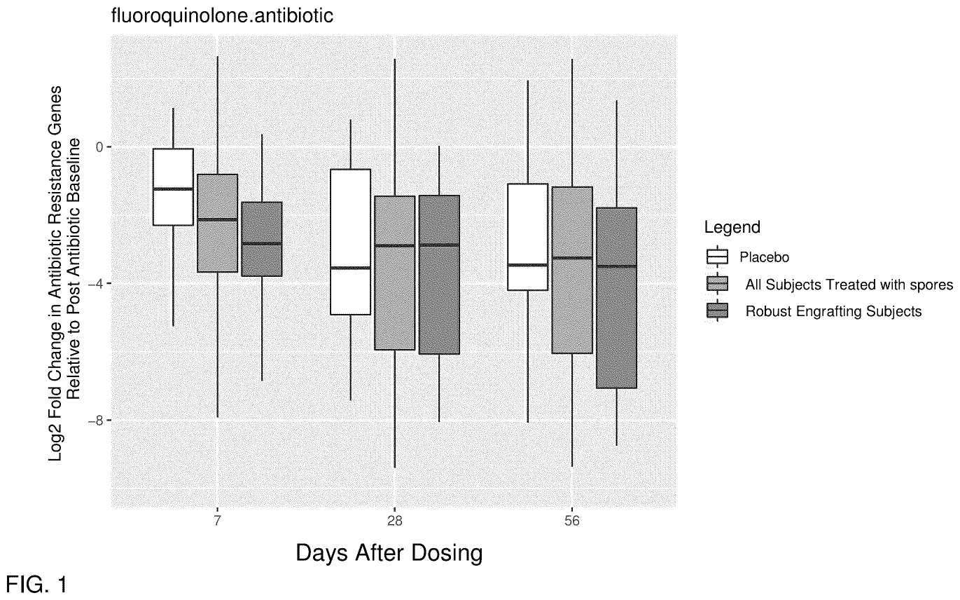

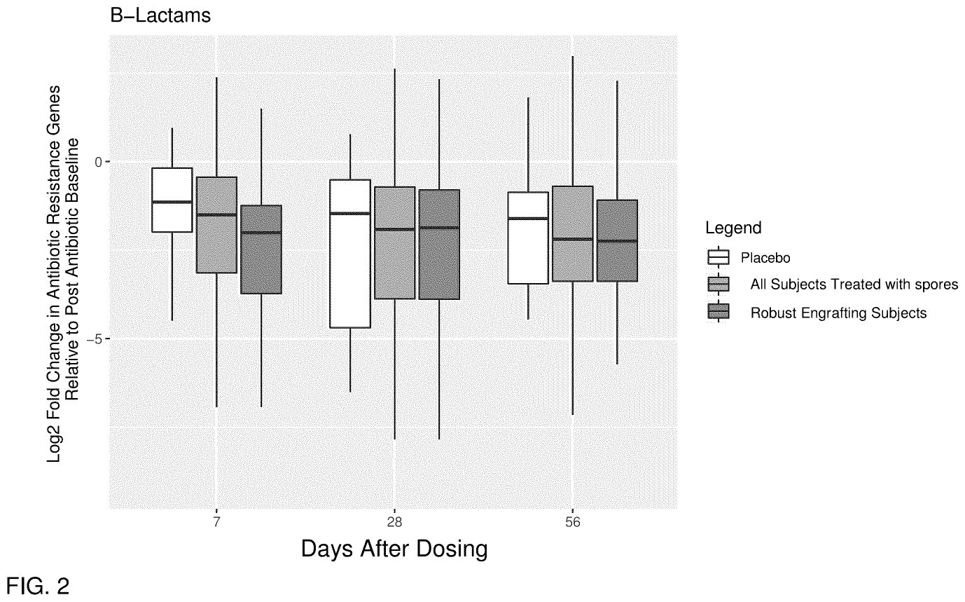

For the Phase 2 study, differential prevalence of spore-former species was observed based on WMS analysis. Gene abundance was assessed using the ShortBRED markers (Kaminski et al., supra) for genes associated with antibiotic resistance identified in ARDB (Liu and Pop, 2009, ARDB-Antibiotic Resistance Genes Database. Nuc Acids Res 37 (Database issue): D443-447). Data were analyzed for a significant engraftment signal, defined as subjects in whom a significant number of species were identified as being significantly genetically similar to species identified in the administered ROAR composition. Analysis of the data resulted in the discovery that in subjects with a significant engraftment signal for the composition, the abundance of genes associated with resistance to multiple classes of drugs, i.e., quinolone drugs (FIG. 1) and beta-lactam or carbapenem drugs (FIG. 2), was reduced.

In additional analyses, for each subject and drug class (from both the Phase 1b and Phase 2 studies), we calculated the log 2 fold change (log 2FC) of drug class marker abundance (DCMA) between samples collected at the visit 1 week after receiving placebo or spores, as compared to samples collected one day prior to treatment with placebo or spores. We compared rCDI subjects who received placebo to rCDI subjects who received spores and in whom significant engraftment was detected. We used the non-parametric Mann-Whitney test to determine if the log 2 FC in DCMA was significantly greater in subjects with engraftment of spores as compared to subjects who received placebo (Table 2). False discovery rates (FDR), the expected proportion of incorrectly rejected null hypotheses for a given p-value due to running multiple tests, was calculated using the Benjamini-Hochberg procedure (p<0.1)

The data demonstrate that a ROAR composition can be used to reduce the abundance of antibiotic resistance genes in a subject.

TABLE 1
Change in abundance of genes in response to treatment

| CARD | | | log2 Engrafters - | Gene |
|---|---|---|---|---|
| accession | p-value | FDR | log2 Placebo | Symbol |
| ARO_3000753 | 1.5E−03 | 8.5E−02 | 0.91 | abeM |
| ARO_3000216 | 2.9E−03 | 8.5E−02 | −1.24 | acrB |
| ARO_3000828 | 2.5E−03 | 8.5E−02 | −1.38 | baeR |
| ARO_3000830 | 1.3E−03 | 8.5E−02 | −1.57 | cpxA |
| ARO_3002867 | 2.7E−03 | 8.5E−02 | 1.35 | dfrF |
| ARO_3004039 | 3.7E−03 | 8.5E−02 | −1.27 | Escherichia |
| | | | | coli emrE |
| ARO_3002522 | 2.4E−04 | 8.5E−02 | 0.90 | novA |
| ARO_3000196 | 1.1E−03 | 8.5E−02 | 1.72 | tet32 |
| ARO_3002871 | 2.2E−03 | 8.5E−02 | 1.46 | tet37 |
| ARO_3000195 | 7.4E−04 | 8.5E−02 | −1.28 | tetB(P) |
| ARO_3002829 | 3.0E−03 | 8.5E−02 | 0.70 | vgaA |
| ARO_3000499 | 5.1E−03 | 9.5E−02 | −1.47 | acrE |
| ARO_3002626 | 6.1E−03 | 9.5E−02 | −0.76 | ANT(6)-Ia |
| ARO_3003838 | 5.7E−03 | 9.5E−02 | −1.50 | gadW |
| ARO_3002836 | 5.6E−03 | 9.5E−02 | −0.50 | InuB |
| ARO_3000263 | 6.7E−03 | 9.5E−02 | −1.33 | marA |
| ARO_3000793 | 6.3E−03 | 9.5E−02 | −1.40 | mdtB |
| ARO_3000794 | 6.7E−03 | 9.5E−02 | −1.59 | mdtC |
| ARO_3000180 | 4.9E−03 | 9.5E−02 | −0.53 | tetA(P) |
| ARO_3000194 | 5.1E−03 | 9.5E−02 | 1.57 | tetW |
| ARO_3003202 | 5.4E−03 | 9.5E−02 | −0.30 | TLA-1 |
| ARO_3000796 | 7.4E−03 | 1.0E−01 | −1.65 | mdtF |
| ARO_3000615 | 8.5E−03 | 1.1E−01 | −1.41 | mefA |
| ARO_3002818 | 8.3E−03 | 1.1E−01 | −1.24 | msrB |
| ARO_3000024 | 9.0E−03 | 1.1E−01 | −1.02 | patA |
| ARO_3000829 | 1.0E−02 | 1.2E−01 | −1.47 | baeS |
| ARO_3000491 | 1.1E−02 | 1.2E−01 | −1.11 | acrD |
| ARO_3000656 | 1.3E−02 | 1.2E−01 | −1.17 | acrS |
| ARO_3000074 | 1.1E−02 | 1.2E−01 | −1.10 | emrB |
| ARO_3000309 | 1.2E−02 | 1.2E−01 | −1.33 | emrD |
| ARO_3000206 | 1.5E−02 | 1.2E−01 | −1.15 | emrK |
| ARO_3000254 | 1.4E−02 | 1.2E−01 | −1.42 | emrY |
| ARO_3000833 | 1.3E−02 | 1.2E−01 | −1.20 | evgS |
| ARO_3000795 | 1.3E−02 | 1.2E−01 | −1.31 | mdtE |
| ARO_3003550 | 1.4E−02 | 1.2E−01 | −1.20 | mdtP |
| ARO_3003950 | 1.3E−02 | 1.2E−01 | −1.48 | msbA |
| ARO_3003952 | 1.4E−02 | 1.2E−01 | −1.12 | yojI |
| ARO_3003549 | 1.5E−02 | 1.3E−01 | −1.14 | mdtO |
| ARO_3002630 | 1.8E−02 | 1.3E−01 | −0.41 | ANT(9)-Ia |
| ARO_3002634 | 1.9E−02 | 1.3E−01 | −1.04 | APH(2″)-Ie |
| ARO_3001328 | 1.9E−02 | 1.3E−01 | −1.02 | Escherichia |
| | | | | coli mdfA |
| ARO_3000832 | 1.8E−02 | 1.3E−01 | −1.18 | evgA |
| ARO_3000676 | 1.7E−02 | 1.3E−01 | −1.08 | H-NS |
| ARO_3003206 | 1.8E−02 | 1.3E−01 | −0.40 | IsaE |
| ARO_3001329 | 2.0E−02 | 1.3E−01 | −1.05 | mdtG |
| ARO_3001216 | 1.8E−02 | 1.3E−01 | −1.12 | mdtH |
| ARO_3003107 | 1.8E−02 | 1.3E−01 | −0.20 | mefB |
| ARO_3002174 | 2.0E−02 | 1.3E−01 | −1.21 | MIR-9 |
| ARO_3000237 | 1.9E−02 | 1.3E−01 | −1.11 | tolC |
| ARO_3000502 | 2.4E−02 | 1.4E−01 | −0.91 | acrF |
| ARO_3002986 | 2.4E−02 | 1.4E−01 | −1.33 | bacA |
| ARO_3002669 | 2.9E−02 | 1.5E−01 | −0.90 | APH(2″)-Ig |
| ARO_3002152 | 2.9E−02 | 1.5E−01 | −1.25 | DHA-21 |
| ARO_3000516 | 2.9E−02 | 1.5E−01 | −1.23 | emrR |
| ARO_3002985 | 3.2E−02 | 1.7E−01 | −0.84 | arnA |
| ARO_3003057 | 3.4E−02 | 1.7E−01 | −0.23 | smeF |
| ARO_3000803 | 3.4E−02 | 1.7E−01 | −0.18 | MexE |
| ARO_3001304 | 3.5E−02 | 1.7E−01 | −0.49 | ErmS |
| ARO_3002559 | 3.6E−02 | 1.7E−01 | −1.06 | AAC(6′)-Ip |
| ARO_3003017 | 3.7E−02 | 1.7E−01 | −0.09 | dfrA21 |
| ARO_3002804 | 3.7E−02 | 1.7E−01 | −0.37 | FosA2 |
| ARO_3002894 | 3.7E−02 | 1.7E−01 | −0.06 | otrC |
| ARO_3003576 | 3.7E−02 | 1.7E−01 | −0.89 | PmrC |
| ARO_3003841 | 3.8E−02 | 1.7E−01 | −0.84 | kdpE |
| ARO_3003578 | 4.0E−02 | 1.8E−01 | −0.87 | pmrF |
| ARO_3002597 | 4.4E−02 | 2.0E−01 | −0.93 | AAC(6′)-Ie- |
| | | | | APH(2″)-Ia |
| ARO_3002635 | 4.4E−02 | 2.0E−01 | −1.22 | APH(2″)-IIa |
| ARO_3000205 | 4.8E−02 | 2.1E−01 | −1.14 | tetX |
| ARO_3000192 | 4.9E−02 | 2.1E−01 | −0.15 | tetS |
| ARO_3004042 | 5.0E−02 | 2.1E−01 | −1.14 | Enterobacter |
| | | | | cloacae acrA |
| ARO_3003773 | 5.6E−02 | 2.3E−01 | −0.04 | Clostridium |
| | | | | perfringens mprF |
| ARO_3002859 | 5.6E−02 | 2.3E−01 | 0.23 | dfrA14 |
| ARO_3003548 | 5.5E−02 | 2.3E−01 | −0.87 | mdtN |
| ARO_3002688 | 5.8E−02 | 2.3E−01 | 0.21 | catS |
| ARO_3002835 | 5.8E−02 | 2.3E−01 | −0.21 | InuA |
| ARO_3000518 | 6.1E−02 | 2.3E−01 | −0.99 | CRP |
| ARO_3000792 | 6.2E−02 | 2.3E−01 | −0.60 | mdtA |
| ARO_3000826 | 6.1E−02 | 2.3E−01 | −0.90 | sdiA |
| ARO_3002671 | 6.3E−02 | 2.3E−01 | −0.32 | cat-TC |
| ARO_3003112 | 6.3E−02 | 2.3E−01 | 0.11 | IsaC |
| ARO_3002670 | 6.9E−02 | 2.5E−01 | 0.70 | cat |
| ARO_3000508 | 6.9E−02 | 2.5E−01 | −0.81 | gadX |
| ARO_3000300 | 7.0E−02 | 2.5E−01 | −0.22 | IsaA |
| ARO_3003699 | 7.0E−02 | 2.5E−01 | −0.25 | mexQ |
| ARO_3000191 | 6.8E−02 | 2.5E−01 | −1.21 | tetQ |
| ARO_3000027 | 7.2E−02 | 2.5E−01 | −0.76 | emrA |
| ARO_3000377 | 7.5E−02 | 2.6E−01 | −0.25 | MexA |
| ARO_3003704 | 8.0E−02 | 2.6E−01 | −0.10 | mexM |
| ARO_3003705 | 8.0E−02 | 2.6E−01 | −0.24 | mexN |
| ARO_3001421 | 7.8E−02 | 2.6E−01 | 0.03 | OXA-26 |
| ARO_3002639 | 8.3E−02 | 2.6E−01 | −0.51 | APH(3″)-Ib |
| ARO_3004072 | 8.5E−02 | 2.6E−01 | −0.22 | OpmB |
| ARO_3003681 | 8.5E−02 | 2.6E−01 | −0.22 | TriC |
| ARO_3002932 | 8.3E−02 | 2.6E−01 | −0.30 | vanSB |
| ARO_3002831 | 8.4E−02 | 2.6E−01 | −1.15 | vgaC |
| ARO_3000250 | 8.9E−02 | 2.7E−01 | −0.27 | ErmC |
| ARO_3002964 | 8.9E−02 | 2.7E−01 | −0.35 | vanWB |
| ARO_3003679 | 9.0E−02 | 2.7E−01 | −0.13 | TriA |
| ARO_3002950 | 9.5E−02 | 2.8E−01 | −0.39 | vanXB |
| ARO_3002553 | 9.8E−02 | 2.9E−01 | −0.02 | AAC(6′)-If |
| ARO_3001777 | 9.8E−02 | 2.9E−01 | −0.04 | OXA-347 |
| ARO_3001303 | 1.1E−01 | 3.0E−01 | 0.07 | ErmO |
| ARO_3000823 | 1.1E−01 | 3.0E−01 | −0.98 | ramA |
| ARO_3002933 | 1.1E−01 | 3.0E−01 | −0.12 | vanSC |
| ARO_3003031 | 1.1E−01 | 3.1E−01 | −0.34 | mexW |
| ARO_3002679 | 1.1E−01 | 3.1E−01 | −0.25 | catB7 |
| ARO_3000578 | 1.2E−01 | 3.4E−01 | −0.21 | CcrA beta- |
| | | | | lactamase |
| ARO_3002156 | 1.2E−01 | 3.4E−01 | 0.16 | FOX-2 |
| ARO_3000811 | 1.2E−01 | 3.4E−01 | −0.04 | mtrD |
| ARO_3001299 | 1.2E−01 | 3.4E−01 | 0.02 | tIrB conferring |
| | | | | tylosinr esistance |
| ARO_3002502 | 1.3E−01 | 3.4E−01 | −0.23 | PDC-5 |
| ARO_3000149 | 1.3E−01 | 3.5E−01 | −0.15 | fosA |
| ARO_3000378 | 1.4E−01 | 3.6E−01 | −0.29 | MexB |
| ARO_3002923 | 1.4E−01 | 3.6E−01 | −0.59 | vanRD |
| ARO_3003577 | 1.4E−01 | 3.7E−01 | −0.65 | pmrE |
| ARO_3003922 | 1.4E−01 | 3.7E−01 | −0.99 | oqxA |
| ARO_3000806 | 1.5E−01 | 3.7E−01 | −0.18 | mexG |
| ARO_3002629 | 1.5E−01 | 3.9E−01 | 0.74 | ANT(6)-Ib |
| ARO_3002868 | 1.6E−01 | 3.9E−01 | −0.25 | dfrG |
| ARO_3000379 | 1.6E−01 | 3.9E−01 | −0.14 | OprM |
| ARO_3002687 | 1.7E−01 | 4.0E−01 | −0.05 | catQ |
| ARO_3002858 | 1.7E−01 | 4.0E−01 | −0.03 | dfrA12 |
| ARO_3000347 | 1.7E−01 | 4.0E−01 | −0.28 | ErmA |
| ARO_3000807 | 1.6E−01 | 4.0E−01 | −0.26 | mexH |
| ARO_3000621 | 1.6E−01 | 4.0E−01 | 0.01 | PC1 |
| | | | | beta-lactamase |
| | | | | (blaZ) |
| ARO_3003046 | 1.7E−01 | 4.0E−01 | −0.03 | qacA |
| ARO_3002965 | 1.6E−01 | 4.0E−01 | −0.26 | vanWG |
| ARO_3002943 | 1.7E−01 | 4.0E−01 | 0.30 | vanHB |
| ARO_3000025 | 1.7E−01 | 4.0E−01 | 0.12 | patB |
| ARO_3004073 | 1.7E−01 | 4.0E−01 | −0.20 | MuxA |
| ARO_3000368 | 1.8E−01 | 4.0E−01 | −0.13 | vanC |
| ARO_3002970 | 1.8E−01 | 4.1E−01 | −0.11 | vanTC |
| ARO_3003698 | 1.8E−01 | 4.1E−01 | −0.21 | mexP |
| ARO_3003835 | 1.9E−01 | 4.2E−01 | −0.24 | cdeA |
| ARO_3004077 | 1.9E−01 | 4.2E−01 | −0.20 | PmpM |
| ARO_3003551 | 1.9E−01 | 4.3E−01 | −0.19 | emeA |
| ARO_3002528 | 2.0E−01 | 4.3E−01 | 0.02 | AAC(3)-Ia |
| ARO_3002578 | 2.0E−01 | 4.3E−01 | −0.24 | AAC(6′)-Ib7 |
| ARO_3002686 | 2.0E−01 | 4.3E−01 | 0.15 | catP |
| ARO_3003693 | 2.0E−01 | 4.3E−01 | −0.19 | mexK |
| ARO_3002452 | 2.0E−01 | 4.3E−01 | −0.79 | OKP-B-19 |
| ARO_3002957 | 2.0E−01 | 4.3E−01 | −0.14 | vanYD |
| ARO_3002921 | 2.0E−01 | 4.4E−01 | −0.38 | vanRB |
| ARO_3001338 | 2.1E−01 | 4.4E−01 | −0.57 | SHV-100 |
| ARO_3004056 | 2.2E−01 | 4.5E−01 | −0.10 | ArmR |
| ARO_3002317 | 2.2E−01 | 4.5E−01 | 0.00 | KPC-7 |
| ARO_3002391 | 2.1E−01 | 4.5E−01 | −0.94 | OXY-1-3 |
| ARO_3000567 | 2.1E−01 | 4.5E−01 | 0.97 | tet(40) |
| ARO_3002185 | 2.2E−01 | 4.5E−01 | −0.06 | MOX-6 |
| ARO_3002972 | 2.2E−01 | 4.5E−01 | −0.11 | vanTG |
| ARO_3001796 | 2.3E−01 | 4.6E−01 | −0.23 | OXA-50 |
| ARO_3002956 | 2.3E−01 | 4.6E−01 | −0.40 | vanYB |
| ARO_3002999 | 2.3E−01 | 4.7E−01 | 0.10 | CblA−1 |
| ARO_3004036 | 2.3E−01 | 4.7E−01 | 0.02 | tetB(60) |
| ARO_3000316 | 2.5E−01 | 5.0E−01 | 0.05 | mphA |
| ARO_3003923 | 2.5E−01 | 5.0E−01 | −0.89 | oqx6 |
| ARO_3002930 | 2.5E−01 | 5.0E−01 | 0.19 | vanRO |
| ARO_3003022 | 2.9E−01 | 5.1E−01 | 0.01 | dfrB3 |
| ARO_3002705 | 2.9E−01 | 5.1E−01 | −0.01 | floR |
| ARO_3002213 | 2.9E−01 | 5.1E−01 | −0.01 | IMP-22 |
| ARO_3004041 | 2.9E−01 | 5.1E−01 | −0.48 | Klebsiella |
| | | | | pneumoniae |
| | | | | acrA |
| ARO_3002482 | 2.9E−01 | 5.1E−01 | −0.05 | LRA-1 |
| ARO_3002486 | 2.9E−01 | 5.1E−01 | 0.01 | LRA-7 |
| ARO_3001214 | 2.6E−01 | 5.1E−01 | −0.56 | mdtM |
| ARO_3000215 | 2.9E−01 | 5.1E−01 | 0.00 | mecR1 |
| ARO_3001610 | 2.9E−01 | 5.1E−01 | 0.01 | OXA-243 |
| ARO_3001503 | 2.9E−01 | 5.1E−01 | 0.03 | OXA-258 |
| ARO_3001780 | 2.7E−01 | 5.1E−01 | 0.08 | OXA-85 |
| ARO_3003836 | 2.9E−01 | 5.1E−01 | 0.01 | qacH |
| ARO_3002828 | 2.7E−01 | 5.1E−01 | −0.19 | srmB |
| ARO_3000573 | 2.9E−01 | 5.1E−01 | 0.02 | tet(43) |
| ARO_3000013 | 2.6E−01 | 5.1E−01 | −0.26 | vanB |
| ARO_3003727 | 2.9E−01 | 5.1E−01 | 0.00 | vanKI |
| ARO_3003724 | 2.9E−01 | 5.1E−01 | −0.01 | vanW1 |
| ARO_3002841 | 2.9E−01 | 5.1E−01 | 0.01 | vatB |
| ARO_3004032 | 3.0E−01 | 5.2E−01 | 0.40 | tetA(46) |
| ARO_3003033 | 3.1E−01 | 5.4E−01 | −0.20 | mexY |
| ARO_3004033 | 3.1E−01 | 5.4E−01 | 0.36 | tetB(46) |
| ARO_3003209 | 3.2E−01 | 5.5E−01 | −0.60 | FosA5 |
| ARO_3002538 | 3.8E−01 | 5.5E−01 | 0.01 | AAC(3)-IIIc |
| ARO_3002540 | 3.8E−01 | 5.5E−01 | 0.00 | AAC(3)-Vla |
| ARO_3002589 | 3.8E−01 | 5.5E−01 | 0.04 | AAC(6′)-Iid |
| ARO_3001816 | 3.4E−01 | 5.5E−01 | −0.24 | ACC-2 |
| ARO_3003848 | 3.8E−01 | 5.5E−01 | −0.02 | ADC-2 |
| ARO_3000780 | 3.8E−01 | 5.5E−01 | 0.02 | adeI |
| ARO_3002646 | 3.8E−01 | 5.5E−01 | 0.01 | APH(3′)-IIc |
| ARO_3002660 | 3.8E−01 | 5.5E−01 | −0.44 | APH(6)-Id |
| ARO_3000838 | 3.8E−01 | 5.5E−01 | −0.03 | arlR |
| ARO_3000839 | 3.8E−01 | 5.5E−01 | −0.02 | arlS |
| ARO_3002847 | 3.8E−01 | 5.5E−01 | −0.04 | arr-2 |
| ARO_3002854 | 3.2E−01 | 5.5E−01 | −0.18 | dfrA1 |
| ARO_3003013 | 3.8E−01 | 5.5E−01 | −0.03 | dfrA15 |
| ARO_3002860 | 3.4E−01 | 5.5E−01 | 0.14 | dfrA17 |
| ARO_3003953 | 3.8E−01 | 5.5E−01 | 0.01 | hmrM |
| ARO_3002259 | 3.8E−01 | 5.5E−01 | −0.02 | IND-3 |
| ARO_3003111 | 3.8E−01 | 5.5E−01 | −0.15 | IsaB |
| ARO_3000800 | 3.2E−01 | 5.5E−01 | −0.17 | MexC |
| ARO_3000808 | 3.5E−01 | 5.5E−01 | −0.14 | mexI |
| ARO_3003692 | 3.3E−01 | 5.5E−01 | −0.16 | mexJ |
| ARO_3003035 | 3.8E−01 | 5.5E−01 | −0.01 | mfpA |
| ARO_3004074 | 3.3E−01 | 5.5E−01 | −0.08 | MuxB |
| ARO_3000802 | 3.8E−01 | 5.5E−01 | −0.12 | OprJ |
| ARO_3000805 | 3.8E−01 | 5.5E−01 | −0.09 | OprN |
| ARO_3001710 | 3.4E−01 | 5.5E−01 | 0.18 | OXA-211 |
| ARO_3001398 | 3.8E−01 | 5.5E−01 | 0.02 | OXA-3 |
| ARO_3000822 | 3.6E−01 | 5.5E−01 | 0.03 | pmrA |
| ARO_3002794 | 3.2E−01 | 5.5E−01 | 0.01 | QnrS5 |
| ARO_3001301 | 3.5E−01 | 5.5E−01 | 0.05 | RlmA(II) |
| ARO_3003056 | 3.8E−01 | 5.5E−01 | −0.01 | smeE |
| ARO_3002493 | 3.8E−01 | 5.5E−01 | −0.01 | SRT-1 |
| ARO_3000168 | 3.7E−01 | 5.5E−01 | −0.04 | tet(D) |
| ARO_3000178 | 3.8E−01 | 5.5E−01 | 0.01 | tet(K) |
| ARO_3000186 | 3.4E−01 | 5.5E−01 | −0.22 | tetM |
| ARO_3000193 | 3.8E−01 | 5.5E−01 | 0.00 | tetT |
| ARO_3000005 | 3.4E−01 | 5.5E−01 | −0.20 | vanD |
| ARO_3002934 | 3.6E−01 | 5.5E−01 | −0.47 | vanSD |
| ARO_3002935 | 3.8E−01 | 5.5E−01 | −0.03 | vanSE |
| ARO_3002953 | 3.8E−01 | 5.5E−01 | −0.01 | vanXM |
| ARO_3004075 | 3.8E−01 | 5.5E−01 | −0.15 | MuxC |
| ARO_3000410 | 3.9E−01 | 5.6E−01 | 0.20 | sul1 |
| ARO_3002598 | 4.0E−01 | 5.7E−01 | −0.17 | ANT(3″)-Ii- |
| | | | | AAC(6′)-IId |
| | | | | fusion protein |
| ARO_3001396 | 4.1E−01 | 5.8E−01 | −0.32 | OXA-1 |
| ARO_3000616 | 4.2E−01 | 5.9E−01 | 0.12 | mel |
| ARO_3004038 | 4.3E−01 | 6.0E−01 | −0.20 | Pseudomonas |
| | | | | aeruginosa |
| | | | | emrE |
| ARO_3000498 | 4.4E−01 | 6.1E−01 | −1.33 | ErmF |
| ARO_3004054 | 4.4E−01 | 6.2E−01 | −0.11 | Pseudomonas |
| | | | | aeruginosa |
| | | | | CpxR |
| ARO_3002982 | 4.6E−01 | 6.4E−01 | −0.11 | amrA |
| ARO_3000593 | 4.6E−01 | 6.4E−01 | 0.19 | ErmQ |
| ARO_3000801 | 4.6E−01 | 6.4E−01 | −0.18 | MexD |
| ARO_3003839 | 4.7E−01 | 6.4E−01 | 0.11 | Mrx |
| ARO_3003680 | 4.8E−01 | 6.6E−01 | −0.25 | TriB |
| ARO_3003030 | 5.2E−01 | 7.0E−01 | −0.17 | mexV |
| ARO_3000167 | 5.3E−01 | 7.1E−01 | 0.02 | tet(C) |
| ARO_3001307 | 5.3E−01 | 7.1E−01 | −0.02 | VgbA |
| ARO_3003954 | 5.4E−01 | 7.2E−01 | 0.00 | efmA |
| ARO_3000804 | 5.4E−01 | 7.2E−01 | 0.04 | MexF |
| ARO_3000412 | 5.5E−01 | 7.3E−01 | −0.25 | sul2 |
| ARO_3003583 | 5.5E−01 | 7.3E−01 | −0.19 | basS |
| ARO_3002684 | 5.6E−01 | 7.3E−01 | −0.03 | catII |
| ARO_3003948 | 5.5E−01 | 7.3E−01 | −0.04 | efrA |
| ARO_3000230 | 5.6E−01 | 7.3E−01 | 0.00 | ANT(2″)-Ia |
| ARO_3002966 | 5.8E−01 | 7.5E−01 | −0.15 | vanXYC |
| ARO_3000190 | 5.8E−01 | 7.5E−01 | 0.35 | tetO |
| ARO_3002645 | 5.9E−01 | 7.5E−01 | −0.11 | APH(3′)-IIb |
| ARO_3003097 | 5.9E−01 | 7.5E−01 | −0.12 | CfxA6 |
| ARO_3000522 | 5.9E−01 | 7.5E−01 | −0.53 | ErmG |
| ARO_3000595 | 5.9E−01 | 7.5E−01 | −0.30 | ErmT |
| ARO_3002881 | 6.0E−01 | 7.5E−01 | 0.03 | ImrC |
| ARO_3002895 | 6.0E−01 | 7.5E−01 | −0.16 | SAT-1 |
| ARO_3003070 | 6.0E−01 | 7.5E−01 | −0.13 | vanXD |
| ARO_3002535 | 6.1E−01 | 7.5E−01 | −0.22 | AAC(3)-IIc |
| ARO_3002539 | 6.1E−01 | 7.5E−01 | −0.29 | AAC(3)-IV |
| ARO_3000174 | 6.1E−01 | 7.5E−01 | 0.01 | tet(G) |
| ARO_3002944 | 6.1E−01 | 7.6E−01 | −0.03 | vanHD |
| ARO_3003717 | 6.4E−01 | 7.8E−01 | −0.05 | ESP-1 |
| ARO_3000181 | 6.4E−01 | 7.8E−01 | 0.01 | tet(V) |
| ARO_3003710 | 6.5E−01 | 7.9E−01 | −0.06 | mexL |
| ARO_3000165 | 6.5E−01 | 7.9E−01 | −0.15 | tet(A) |
| ARO_3002931 | 6.5E−01 | 7.9E−01 | −0.03 | vanSA |
| ARO_3000010 | 6.7E−01 | 8.1E−01 | −0.03 | vanA |
| ARO_3002605 | 6.7E−01 | 8.1E−01 | 0.00 | aadA5 |
| ARO_3001933 | 7.1E−01 | 8.5E−01 | −0.63 | CTX-M-72 |
| ARO_3000175 | 7.1E−01 | 8.5E−01 | 0.01 | tet(H) |
| ARO_3002556 | 7.1E−01 | 8.5E−01 | 0.05 | AAC(6′)-Ii |
| ARO_3002680 | 7.2E−01 | 8.6E−01 | −0.25 | catB8 |
| ARO_3000809 | 7.3E−01 | 8.6E−01 | −0.14 | opmD |
| ARO_3002897 | 7.3E−01 | 8.6E−01 | −0.37 | SAT-4 |
| ARO_3001313 | 7.3E−01 | 8.6E−01 | 0.00 | facT |
| ARO_3003197 | 7.4E−01 | 8.7E−01 | −0.06 | aadA25 |
| ARO_3003949 | 7.7E−01 | 9.0E−01 | 0.00 | efrB |
| ARO_3003682 | 7.7E−01 | 9.0E−01 | −0.13 | OpmH |
| ARO_3001959 | 7.8E−01 | 9.0E−01 | −0.45 | CTX-M-100 |
| ARO_3002942 | 7.9E−01 | 9.1E−01 | −0.02 | vanHA |
| ARO_3003559 | 8.1E−01 | 9.3E−01 | −0.29 | cepA beta- |
| | | | | lactamase |
| ARO_3002628 | 8.2E−01 | 9.4E−01 | −0.02 | aad(6) |
| ARO_3000596 | 8.2E−01 | 9.4E−01 | 0.02 | ErmX |
| ARO_3002949 | 8.2E−01 | 9.4E−01 | −0.05 | vanXA |
| ARO_3002837 | 8.3E−01 | 9.4E−01 | −0.30 | InuC |
| ARO_3002756 | 8.3E−01 | 9.4E−01 | 0.21 | QnrB41 |
| ARO_3002875 | 8.4E−01 | 9.4E−01 | 0.04 | dfrE |
| ARO_3002647 | 8.5E−01 | 9.5E−01 | 0.00 | APH(3′)-IIIa |
| ARO_3000179 | 8.5E−01 | 9.5E−01 | −0.21 | tet(L) |
| ARO_3002069 | 8.6E−01 | 9.6E−01 | −0.09 | CMY-59 |
| ARO_3002019 | 9.0E−01 | 9.8E−01 | 0.01 | CMY-8 |
| ARO_3003095 | 9.0E−01 | 9.8E−01 | 0.12 | imiS |
| ARO_3002225 | 9.0E−01 | 9.8E−01 | −0.03 | IMP-34 |
| ARO_3001413 | 9.0E−01 | 9.8E−01 | 0.01 | OXA-18 |
| ARO_3000565 | 9.0E−01 | 9.8E−01 | 0.00 | tet(38) |
| ARO_3000177 | 8.8E−01 | 9.8E−01 | −0.25 | tet(J) |
| ARO_3002882 | 9.1E−01 | 9.8E−01 | 0.07 | ImrD |
| ARO_3002363 | 9.1E−01 | 9.8E−01 | 0.02 | PER-1 |
| ARO_3003069 | 9.1E−01 | 9.8E−01 | −0.02 | vanXYG |
| ARO_3002819 | 9.2E−01 | 9.9E−01 | −0.03 | msrC |
| ARO_3003210 | 9.4E−01 | 9.9E−01 | −0.27 | FosA4 |
| ARO_3000026 | 9.4E−01 | 9.9E−01 | −0.01 | mepA |
| ARO_3000746 | 9.4E−01 | 9.9E−01 | 0.00 | mepR |
| ARO_3000815 | 9.4E−01 | 9.9E−01 | 0.00 | mgrA |
| ARO_3003199 | 1.0E+00 | 1.0E+00 | 0.02 | AAC(6′)-Iak |
| ARO_3002613 | 9.6E−01 | 1.0E+00 | 0.01 | aadA13 |
| ARO_3002641 | 9.7E−01 | 1.0E+00 | −0.07 | APH(3′)-Ia |
| ARO_3002655 | 1.0E+00 | 1.0E+00 | −0.24 | APH(4)-Ia |
| ARO_3003730 | 1.0E+00 | 1.0E+00 | −0.06 | Bifidobacteria |
| | | | | intrinsic |
| | | | | ileS conferring |
| | | | | resistance to |
| | | | | mupirocin |
| ARO_3001920 | 9.8E−01 | 1.0E+00 | −0.09 | CTX-M-59 |
| ARO_3003955 | 9.9E−01 | 1.0E+00 | −0.03 | efpA |
| ARO_3000375 | 9.6E−01 | 1.0E+00 | 0.07 | ErmB |
| ARO_3004085 | 1.0E+00 | 1.0E+00 | −0.06 | InuG |
| ARO_3002665 | 9.9E−01 | 1.0E+00 | −0.16 | npmA |
| ARO_3003700 | 9.9E−01 | 1.0E+00 | −0.18 | opmE |
| ARO_3002788 | 1.0E+00 | 1.0E+00 | −0.04 | QnrD1 |
| ARO_3001388 | 9.6E−01 | 1.0E+00 | −0.37 | TEM-211 |
| ARO_3000556 | 9.5E−01 | 1.0E+00 | −0.01 | tet44 |
| ARO_3004035 | 9.7E−01 | 1.0E+00 | −0.01 | tetA(60) |
| ARO_3002909 | 1.0E+00 | 1.0E+00 | 0.00 | vanG |
| ARO_3002919 | 9.9E−01 | 1.0E+00 | −0.08 | vanRA |
| ARO_3002922 | 1.0E+00 | 1.0E+00 | −0.06 | vanRC |
| ARO_3002955 | 9.5E−01 | 1.0E+00 | 0.01 | vanYA |
| ARO_3002962 | 1.0E+00 | 1.0E+00 | −0.09 | vanZA |

TABLE 2
Engraftment of spores is associated with significant
reduction in antibiotic resistance genes

| | log2FC high | | | Examples of |
|---|---|---|---|---|
| | engrafters - | | | antibiotics in drug |
| Drug Class | 1og2 Placebo | p-value | FDR | class |
| Aminocoumarin | −1.534 | 0.003 | 0.035 | Novobiocin, |
| antibiotic | | | | Albamycin, |
| | | | | Coumemycin, |
| | | | | Clorobiocin |
| Aminoglycoside | −1.241 | 0.009 | 0.035 | Kanamycin, Amikacin, |
| antibiotic | | | | Tobramycin, Dibekacin, |
| | | | | Gentamicin, Sisomicin, |
| | | | | Netilmicin, Neomycins |
| | | | | (B, C, E) |
| | | | | Streptomycin |
| cephalosporin | −1.428 | 0.014 | 0.035 | Cefacetrill, Cefradrin, |
| | | | | Cefroxadin, |
| | | | | Cefaloglycin, Cefaclor, |
| | | | | Cefalexin, Cefadroxil, |
| | | | | Cefatrizin, Cefazedon, |
| | | | | Cefapirin, Ceftezol, |
| | | | | Cefazolin, Cefazaflur, |
| | | | | Cefalotin, Cefaloridin, |
| | | | | Cefalonium |
| cephamycin | −1.214 | 0.008 | 0.035 | Cefoxitin, Cefotetan, |
| | | | | Cefmetazole |
| Fluoroquinolone | −1.401 | 0.007 | 0.035 | Flumequine, oxolinic |
| antibiotic | | | | acid, rosoxacin, |
| | | | | ciprofloxacin, |
| | | | | fleroxacin, |
| | | | | lomefloxacin, |
| | | | | nadifloxacin, |
| | | | | norfloxacin, ofloxacin, |
| | | | | pefloxacin, rufloxacin, |
| | | | | balofloxacin, |
| | | | | grepafloxacin, |
| | | | | levofloxacin, |
| | | | | pazufloxacin, |
| | | | | sparfloxacin, |
| | | | | temafloxacin, |
| | | | | clinafloxacin, |
| | | | | gatifloxacin, |
| | | | | moxifloxacin, |
| | | | | sitafloxacin, |
| | | | | prulifloxacin, |
| | | | | besifloxacin, |
| | | | | delafloxacin, |
| | | | | ozenoxacin, |
| | | | | danofloxacin, |
| | | | | difloxacin, |
| | | | | enrofloxacin, |
| | | | | ibafloxacin, |
| | | | | marbofloxacin, |
| | | | | orbifloxacin, |
| | | | | sarafloxacin |
| glycylcycline | −1.658 | 0.009 | 0.035 | Tigercycline |
| Macrolide | −1.288 | 0.002 | 0.035 | Erythromycin, |
| antibiotic | | | | Clarithromycin, |
| | | | | Roxithromycin |
| Nitroimidazole | −1.477 | 0.013 | 0.035 | Nitroimidazole |
| antibiotic | | | | |
| Nucleoside | −1.347 | 0.011 | 0.035 | Tunicamycin, |
| antibiotic | | | | Streptothricin |
| penam | −1.424 | 0.010 | 0.035 | Benzylpenicillin |
| Peptide antibiotic | −1.279 | 0.006 | 0.035 | |
| Phenicol antibiotic | −1.139 | 0.012 | 0.035 | chloramphenicol |
| Rifamycin | −1.470 | 0.006 | 0.035 | rifamycin |
| antibiotic | | | | |
| Benzalkonium | −1.016 | 0.019 | 0.035 | Benzalkonium chloride |
| chloride | | | | |
| monobactam | −1.114 | 0.018 | 0.035 | Aztreonam |
| penem | −1.155 | 0.015 | 0.035 | Faropenem |
| rhodamine | −1.016 | 0.019 | 0.035 | rhodamine |
| triclosan | −1.234 | 0.018 | 0.035 | triclosan antibiotics |
| Lincosamide | −1.123 | 0.021 | 0.037 | lincomycin, |
| antibiotic | | | | clindamycin, pirlimycin |
| Streptogramin | −1.065 | 0.035 | 0.057 | streptogramin |
| antibiotic | | | | |
| Acridine dye | −0.905 | 0.043 | 0.067 | |
| carbapenem | −1.239 | 0.052 | 0.075 | carbapenem |
| Tetracycline | −0.558 | 0.050 | 0.075 | tetracycline |
| antibiotic | | | | |
| fosfomycin | −1.024 | 0.066 | 0.091 | fosfomycin |

### Example 2: Bacteria Associated with Changes in Abundance of Abx Resistance Genes

Computational analysis to determine whether the presence of specific bacterial species is predictive of a decrease in the abundance of antibiotic resistance genes was performed. Species identified in these analyses can be used in designed Abx compositions. To identify microbial species that are associated with a reduction of antimicrobial resistance genes, we calculated the Spearman correlation between species abundance and the DCMA of drug classes showing a significant reduction in subjects in whom engraftment of spores was observed relative to placebo. P-values were adjusted to account for multiple hypothesis testing using the Benjamini-Hochberg procedure (p<0.1) as described supra. The set of species associated with significant reduction of at least one drug of antibiotic resistance genes is summarized in Table 3. We have further defined the set of species positively correlated with elevated abundances of at least one class of antibiotic resistance genes, summarized in Table 4. Dose species in all tables are bacterial species identified in dose lots administered to subjects. Full length 16S sequences of species described herein are provided in Tables 5 (negative correlates) and 6 (positive correlates). In some embodiments, the bacterium (a) are from a genus listed below. Those of skill in the art can use standard methods to identify the genera.

TABLE 3
Species significantly correlated with reduction of at least one class of antibiotic resistance genes

| | Number of | | | | Representative |
|---|---|---|---|---|---|
| | drug classes | | | | Genome |
| | with | Spore- | Dose | Exemplary | Accession |
| | significant | former | Species | sequence | Number(s) |
| Species | reduction | (Y/N) | (Y/N) | identifier | (NCBI) |
| Anaerofustis_stercorihominis | 21 | Y | Y | 96, 97 | GCF_000154825 |
| Anaerostipes_caccae | 20 | Y | N | 62-66 | GCF_000154305 |
| Anaerostipes_hadrus | 23 | Y | Y | 223 | GCF_000332875 |
| Anaerostipes_sp. | 24 | Y | Y | 24 | GCA_900066985 |
| Anaerostipes_unclassified | 21 | N | N | | |
| Anaerotruncus_colihominis | 24 | Y | Y | 89-92 | GCF_000154565 |
| Anaerotruncus_unclassified | 22 | N | N | | |
| Bacteroides_fragilis | 18 | N | N | 184-191 | GCF_000273095 |
| Bacteroides_uniformis | 6 | N | N | 192-198 | GCF_000273275 |
| Bacteroides_vulgatus | 16 | N | N | 199-204 | GCF_000273295 |
| Blautia_faecis (a) | 22 | Y | Y | 9 | GCA_900066145 |
| Blautia_faecis (b) | 24 | Y | Y | 22 | GCA_900066945 |
| Blautia_hydrogenotrophica | 22 | Y | Y | 101 | GCF_000157975 |
| Blautia_producta | 21 | Y | Y | 281 | GCF_000373885 |
| Blautia_sp. | 22 | Y | Y | 268, 269 | |
| Blautia_sp_CAG_257 | 22 | Y | Y | 261 | |
| Blautia_wexlerae | 24 | Y | Y | 104-110, | GCF_000159975 |
| | | | | 255 | |
| Clostridiaceae_bacterium_JC118 | 21 | Y | Y | 222 | GCF_000313565 |
| Clostridiales_bacterium_1_7_47FAA | 23 | Y | Y | 98 | GCF_000155435 |
| Clostridium_aldenense | 21 | Y | Y | 278 | |
| Clostridium_asparagiforme | 22 | Y | Y | 103 | GCF_000158075 |
| Clostridium_bardettii | 24 | Y | Y | 80-85 | GCF_000154445 |
| Clostridium_bolteae | 23 | Y | Y | 73 | GCF_000154365 |
| Clostridium_clostridioforme | 21 | Y | Y | 231-233 | GCF_000371525 |
| Clostridium_hathewayi | 24 | Y | Y | 229, 230 | GCF_000371445 |
| Clostridium_lavalense | 20 | Y | Y | 280 | |
| Clostridium_leptum | 22 | Y | Y | 71, 72 | GCF_000154345 |
| Clostridium_nexile | 24 | Y | N | 282 | GCF_000156035 |
| Clostridium_ramosum | 21 | Y | Y | 86 | GCF_000154485 |
| Clostridium_sp. | 21 | Y | Y | 21 | GCA_900066915 |
| Clostridium_sp. | 22 | Y | Y | 18 | GCA_900066805 |
| Clostridium_sp. | 22 | Y | Y | 6 | GCA_900066055 |
| Clostridium_sp. | 22 | Y | Y | 12 | GCA_900066535 |
| Clostridium_sp. | 23 | Y | Y | 11 | GCA_900066365 |
| Clostridium_sp. | 24 | Y | Y | 8 | GCA_900066135 |
| Clostridium_sp. | 24 | Y | Y | | |
| Clostridium_sp. | 23 | Y | Y | 17 | GCA_900066795 |
| Clostridium_sp. | 22 | Y | Y | 15, 16 | GCA_900066785 |
| Clostridium_sp. | 23 | Y | Y | 10 | GCA_900066155 |
| Clostridium_scindens | 21 | Y | Y | 87, 88 | GCF_000154505 |
| Clostridium_sp_CAG_91 | 23 | Y | Y | 266 | |
| Clostridium_sp_KLE_1755 | 22 | Y | N | 247 | GCF_000466465 |
| Clostridium_symbiosum | 23 | Y | Y | 248 | GCF_000466485 |
| Coprobacillus_unclassified | 21 | N | N | | |
| Coprococcus_catus | 22 | Y | Y | 143 | GCF_000210555 |
| Coprococcus_comes | 21 | Y | Y | 99 | GCF_000155875 |
| Dialister_invisus | 21 | N | N | 111-113 | GCF_000160055 |
| Dorea_formicigenerans | 22 | Y | Y | 114-119 | GCF_000169235 |
| Dorea_longicatena | 21 | Y | Y | 60, 61 | GCF_000154065 |
| Eggerthella_lenta | 22 | N | N | 39-41 | GCF_000024265 |
| Eggerthella_unclassifted | 20 | N | N | | |
| Emergencia_timonensis | 22 | Y | Y | 14 | GCA_900066695 |
| Erysipelotrichaceae_bacterium_21_3 | 21 | N | N | 164-168 | GCF_000242195 |
| Erysipelotrichaceae_bacterium_6_1_45 | 21 | N | N | 160-163 | GCF_000242175 |
| Eubacterium_contortum | 23 | Y | Y | 272-275 | |
| Eubacterium_eligens | 22 | Y | Y | 43-47 | GCF_000146185 |
| Eubacterium_hallii | 22 | Y | Y | 120 | GCF_000173975 |
| Eubacterium_ramulus | 22 | Y | Y | 249 | GCF_000469345 |
| Eubacterium_rectale | 23 | Y | Y | 34-38 | GCF_000020605 |
| Eubacterium_sp. | 21 | Y | Y | | |
| Eubacterium_sp_3_1_31 | 20 | Y | Y | 205-209, | GCF_000273585 |
| | | | | 262 | |
| Eubacterium_ventriosum | 23 | Y | Y | 48 | GCF_000153885 |
| Faecalibacterium_prausnitzii | 24 | Y | Y | 74-79 | GCF_000154385 |
| Flavonifractor_plautii | 23 | Y | Y | 158 | GCF_000239295 |
| Flavonifractor_sp. (a) | 21 | Y | Y | 19 | GCA_900066825 |
| Flavonifractor_sp. (b) | 23 | Y | Y | 13 | GCA_900066645 |
| Fusicatenibacter_saccharivorans | 23 | Y | Y | 4 | GCA_001405555 |
| Gemmiger_formicilis_(a) | 24 | Y | Y | 256 | |
| Gemmiger_formicilis_(b) | 24 | Y | Y | 263 | |
| Holdemania_filiformis | 24 | Y | Y | 102 | GCF_000157995 |
| Holdemania_unclassified | 24 | N | N | | |
| Intestinimonas_butyriciproducens | 23 | Y | Y | 264 | |
| Intestinimonas_massiliensis | 22 | N | N | 20 | GCA_900066895 |
| Lachnospira_pectinoschiza | 24 | Y | Y | 5 | GCA_001405835 |
| Lachnospira_pectinoschiza | 19 | Y | Y | 270 | |
| Lachnospiraceae_bacterium_1_1_57FAA | 23 | Y | Y | 150 | GCF_000218445 |
| Lachnospiraceae_bacterium_1_4_56FAA | 21 | Y | Y | 145 | GCF_000218385 |
| Lachnospiraceae_bacterium_2_1_58FAA | 23 | Y | Y | 151 | GCF_000218465 |
| Lachnospiraceae_bacterium_3_1_46FAA | 22 | Y | Y | 139 | GCF_000209405 |
| Lachnospiraceae_bacterium_3_1_57FAA_CT1 | 22 | Y | Y | 146-148 | GCF_000218405 |
| Lachnospiraceae_bacterium_4_1_37FAA | 18 | Y | N | 137 | GCF_000191805 |
| Lachnospiraceae_bacterium_5_1_57FAA | 22 | Y | Y | 149 | GCF_000218425 |
| Lachnospiraceae_bacterium_5_1_63FAA | 24 | Y | Y | 134 | GCF_000185525 |
| Lachnospiraceae_bacterium_7_1_58FAA | 23 | Y | Y | 159 | GCF_000242155 |
| Lachnospiraceae_bacterium_8_1_57FAA | 21 | Y | Y | 135 | GCF_000185545 |
| Lachnospiraceae_bacterium_9_1_43BFAA | 24 | Y | Y | 141 | GCF_000209445 |
| Lactonifactor_longoviformis | 23 | Y | Y | 271 | |
| Oscillibacter_unclassified | 23 | N | N | | |
| Oscillospiraceae_bacterium_VE202_24 | 23 | N | N | 265 | |
| Parabacteroides_unclassified | 18 | N | N | | |
| Prevotella_buccae | 22 | N | N | 133 | GCF_000184945 |
| Roseburia_faecis | 23 | Y | Y | 259 | |
| Roseburia_hominis | 21 | Y | Y | 152-155 | GCF_000225345 |
| Roseburia_intestinalis | 23 | Y | Y | 100 | GCF_000156535 |
| Roseburia_inulinivorans | 24 | Y | Y | 283 | GCF_000174195 |
| Ruminococcus_bicirculans | 23 | Y | Y | 25, 26 | GCA_900067025 |
| Ruminococcus_bromii | 22 | Y | Y | 142 | GCF_000209875 |
| Ruminococcus_gnavus | 23 | Y | Y | 250-254, | GCF_000507805 |
| | | | | 277 | |
| Ruminococcus_obeum_(a) | 23 | Y | Y | 49-53 | GCF_000153905 |
| Ruminococcus_obeum_(b) | 19 | Y | Y | 267 | |
| Ruminococcus_sp. | 22 | Y | Y | 7, 258 | GCA_900066095 |
| Ruminococcus_torques | 24 | Y | Y | 54-59 | GCF_000153925 |
| Streptococcus_parasanguinis | 21 | N | N | 170-173 | GCF_000262145 |
| Streptococcus_salivarius | 21 | N | N | 210-216 | GCF_000286295 |
| Subdoligranulum_unclassified | 23 | N | N | | |
| Sutterella_wadsworthensis | 9 | N | N | 243-246 | GCF_000411515 |
| Bacteroides_thetaiotaomicron | 1 | N | N | 239-242 | GCF_000403155 |
| Haemophilus_parainfluenzae | 7 | N | N | 169 | GCF_000261285 |
| Parabacteroides_distasonis | 2 | N | N | 217-221 | GCF_000307435 |
| Akkermansia_muciniphila | 17 | N | N | 31-33 | GCF_000020225 |
| Bacteroides_dorei | 11 | N | N | 174-183 | GCF_000273055 |
| Butyrivibrio_sp. | 15 | N | N | 260 | |
| Clostridium_citroniae | 20 | Y | Y | 157 | GCF_000233455 |
| Clostridium_difficile | 8 | Y | N | 1-3 | GCA_000451045 |
| Clostridium_disporicum | 9 | Y | Y | 23 | GCA_900066955 |
| Clostridium_innocuum | 4 | Y | Y | 224-228 | GCF_000371425 |
| Clostridium_spiroforme | 11 | Y | Y | 93-95 | GCF_000154805 |
| Dorea_unclassified | 13 | N | N | | |
| Eisenbergiella_massiliensis | 11 | Y | Y | 257 | |
| Eisenbergiella_tayi_sp. | 14 | Y | Y | 276 | |
| Erysipelotrichaceae_bacterium_2_2_44A | 20 | N | N | 156 | GCF_000225685 |
| Eubacterium_siraeum | 14 | Y | Y | 67-70 | GCF_000154325 |
| Lachnospiraceae_bacterium_6_1_63FAA | 11 | Y | N | 140 | GCF_000209425 |
| Ruminococcaceae_bacterium_D16 | 19 | Y | Y | 121 | |
| Ruminococcus_gnavus | 7 | Y | Y | 250-254, | GCF_000507805 |
| | | | | 277 | |
| Ruminococcus_sp. | 12 | Y | Y | 7, 258 | GCF_000507805 |
| Streptococcus_australis | 4 | N | N | 136 | GCF_000186465 |
| Streptococcus_gordonii | 3 | N | N | 27-30 | GCF_000017005 |
| Streptococcus_infantis | 5 | N | N | 284-286 | GCF_000215385, |
| | | | | | GCF_000187465, |
| | | | | | GCF_000223335, |
| | | | | | GCF_000223255, |
| | | | | | GCF_000260755 |
| Streptococcus_sanguinis | 5 | N | N | 138 | GCF_000192205 |
| Dysgonomonas_mossii | 6 | N | N | 144 | GCF_000213575 |
| Streptococcus_thermophilus | 1 | N | N | 128-132 | GCF_000182875 |
| Peptostreptococcaceae_noname_unclassifted | 10 | N | N | | |
| Turicibacter_unclassified | 6 | N | N | | |
| Burkholderiales_bacterium_1_1_47 | 3 | N | N | 42 | GCF_000144975 |
| Clostridium_sp_CL_6 | 4 | Y | N | 287 | |
| Desulfovibrio_desulfuricans | 1 | N | N | | GCF_000384815, |
| | | | | | GCF_000022125, |
| | | | | | GCF_000420465, |
| | | | | | GCF_000189295 |
| Lactobacillus_fermentum | 1 | N | N | 234-238 | GCF_000397165 |
| Streptococcus_vestibularis | 1 | N | N | 122-127 | GCF_000180075 |

TABLE 4
Species significantly positively correlated with elevated abundances of at least one class of antibiotic
resistance genes

| | Number of | | | | |
|---|---|---|---|---|---|
| | drug classes | | | | |
| | associated | | | | Representative |
| | with | | | | Genome |
| | increasing | Spore- | Dose | Exemplary | Accession |
| | species | former | Species | sequence | Number(s) |
| Species | abundance | (Y/N) | (Y/N) | identifier | (NCBI) |
| Campylobacter_curvus | 20 | N | N | 367 | GCF_000376325 |
| Citrobacter_freundii | 22 | N | N | 361, 362 | GCF_000312465 |
| Citrobacter_unclassified | 22 | N | N | | |
| Desulfovibrio_desulfuricans | 6 | N | N | | GFC_000384815, |
| | | | | | GCF_000022125, |
| | | | | | GCF_000420465, |
| | | | | | GCF_000189295 |
| Enterobacter_cloacae | 22 | N | N | 387 | GCF_000467655 |
| Enterococcus_faecalis | 21 | N | N | | GCF_000393515, |
| | | | | | GCF_000394095, |
| | | | | | GCF_000393615, |
| | | | | | GCF_000157495, |
| | | | | | GCF_000390625, |
| | | | | | GCF_000390925, |
| | | | | | GCF_000148005, |
| | | | | | GCF_000148065, |
| | | | | | GCF_000394075, |
| | | | | | GCF_000393495, |
| | | | | | GCF_000391465, |
| | | | | | GCF_000392675, |
| | | | | | GCF_000396365, |
| | | | | | GCF_000157475, |
| | | | | | GCF_000396665, |
| | | | | | GCF_000148245, |
| | | | | | GCF_000393155, |
| | | | | | GCF_000148165, |
| | | | | | GCF_000391165, |
| | | | | | GCF_000157515, |
| | | | | | GCF_000396085, |
| | | | | | GCF_000395285, |
| | | | | | GCF_000479065, |
| | | | | | GCF_000393095, |
| | | | | | GCF_000393255, |
| | | | | | GCF_000391065, |
| | | | | | GCF_000394955, |
| | | | | | GCF_000415065, |
| | | | | | GCF_000394975, |
| | | | | | GCF_000415085, |
| | | | | | GCF_000395405, |
| | | | | | GCF_000172575, |
| | | | | | GCF_000390525, |
| | | | | | GCF_000393115, |
| | | | | | GCF_000394775, |
| | | | | | GCF_000393475, |
| | | | | | GCF_000390705, |
| | | | | | GCF_000205205, |
| | | | | | GCF_000395135, |
| | | | | | GCF_000392815, |
| | | | | | GCF_000320305, |
| | | | | | GCF_000396205, |
| | | | | | GCF_000390665, |
| | | | | | GCF_000390685, |
| | | | | | GCF_000391725, |
| | | | | | GCF_000163815, |
| | | | | | GCF_000391605, |
| | | | | | GCF_000391445, |
| | | | | | GCF_000391425, |
| | | | | | GCF_000396185, |
| | | | | | GCF_000148445, |
| | | | | | GCF_000148125, |
| | | | | | GCF_000396605, |
| | | | | | GCF_000396045, |
| | | | | | GCF_000291565, |
| | | | | | GCF_000394315, |
| | | | | | GCF_000395035, |
| | | | | | GCF_000147455, |
| | | | | | GCF_000391345, |
| | | | | | GCF_000396525, |
| | | | | | GCF_000392755 |
| Escherichia_coli | 24 | N | N | 388-394 | GCF_000474825 |
| Escherichia_unclassified | 23 | N | N | | |
| Fusobacterium_nucleatum | 15 | N | N | 403 | GCF_000479185 |
| Klebsiella_oxytoca | 22 | N | N | 330 | GCF_000252915 |
| Klebsiella_pneumoniae | 22 | N | N | 395-402 | GCF_000474865 |
| Klebsiella_unclassified | 21 | N | N | | |
| Lactobacillus_animalis | 22 | N | N | 317 | GCF_000183825 |
| Lactobacillus_casei_paracasei | 22 | N | N | 378, 379, | GCF_000409835, |
| | | | | 381-385 | GCF_000418515 |
| Lactobacillus_plantarum | 22 | N | N | 386 | GCF_000463075 |
| Lactobacillus_rhamnosus | 24 | N | N | 328, 329 | GCF_000235785 |
| Lactobacillus_salivarius | 9 | N | N | 303 | GCF_000159395 |
| Lactococcus_lactis | 15 | N | N | 363, 364 | GCF_000348965 |
| Megasphaera_micronuciformis | 22 | N | N | 310 | GCF_000165735 |
| Megasphaera_unclassified | 11 | N | N | | |
| Morganella_morganii | 24 | N | N | 355 | GCF_000286435 |
| Pantoea_unclassified | 24 | N | N | | |
| Pediococcus_acidilactici | 19 | N | N | 299 | GCF_000146325 |
| Proteus_penneri | 17 | N | N | 301, 302 | GCF 000155835 |
| Streptococcus_mitis_oralis_pneumoniae | 3 | N | N | 288-291, | GCF_000014365, |
| | | | | 332 | GCF_000257865 |
| Streptococcus_thermophilus | 5 | N | N | 312-316 | GCF_000182875 |
| Veillonella_atypica | 19 | N | N | 311 | GCF_000179755 |
| Veillonella_dispar | 18 | N | N | 304-306 | GCF_000160015 |
| Veillonella_parvula | 13 | N | N | 322 | GCF_000215025 |
| Bifidobacterium_dentium | 1 | N | N | 300 | GCF_000146775 |
| Butyrivibrio_sp. | 1 | N | N | 404 | |
| Clostridium_sp_CL_6 | 1 | Y | N | 287 | NCBI Taxonomy |
| | | | | | ID 1499683 |
| Enterococcus_faecium | 18 | N | N | 380 | GCF_000414965 |
| Erysipelotrichaceae_bacterium_2_2_44A | 1 | N | N | 326 | GCF_000225685 |
| Lactobacillus_fermentum | 2 | N | N | 368-372 | GCF_000397165 |
| Lactobacillus_gasseri | 1 | N | N | 377 | GCF_000406345 |
| Proteus_mirabilis | 8 | N | N | 292-298 | GCF_000069965 |
| Rothia_dentocariosa | 5 | N | N | 307-309 | GCF 000164695 |
| Proteus_unclassified | 5 | N | N | | |
| Veillonella_unclassified | 14 | N | N | | |
| Alistipes_onderdonkii | 1 | N | N | 365, 366 | GCF_000374505 |
| Bacteroides_ovatus | 5 | N | N | 333-336 | GCF_000273195 |
| Bacteroides_vulgatus | 4 | N | N | 344-349 | GCF_000273295 |
| Bilophila_unclassified | 3 | N | N | | |
| Bilophila_wadsworthia | 3 | N | N | 318-321 | GCF_000185705 |
| Parabacteroides_distasonis | 6 | N | N | 356-360 | GCF_000307435 |
| Parabacteroides_unclassified | 3 | N | N | | |
| Alistipes_unclassified | 2 | N | N | | |
| Rothia_mucilaginosa | 1 | N | N | 327 | GCF_000231235 |
| Bacteroides_thetaiotaomicron | 1 | N | N | 373-376 | GCF_000403155 |
| Bacteroides_uniformis | 2 | N | N | 337-343 | GCF_000273275 |
| Eggerthella_unclassified | 1 | N | N | | |
| Eubacterium_sp_3_1_31 | 2 | Y | Y | 350-354 | GCF_000273585 |
| Lachnospiraceae_bacterium_3_1_57FAA_CT1 | 1 | Y | Y | 323-325 | GCF_000218405 |
| Ruminococcus_obeum | 1 | Y | Y | 405 | |
| Turicibacter_unclassified | 1 | N | N | | |
| Clostridium_perfringens | 1 | Y | Y | 331 | GCF_000255475 |

TABLE 5
Full length 16S rDNA sequences of species (negative correlates)

| Species | Sequence | |
|---|---|---|
| Name | Identifier | 16S rDNA sequence |
| Clostridium_ | SEQ ID | ttatgagagtttgatcctggctcaggacgaacgctggcggcgtgcctaatacatgcaagtcgaacgcttctttttccaccggagc |
| difficile | NO: 1 | ttgctccaccggaaaaagaggagtggcgaacgggtgagtaacacgtgggtaacctgcccatcagaaggggataacacttgg |
| | | aaacaggtgctaataccgtataacaatcgaaaccgcatggttttgatttgaaaggcgctttcgggtgtcgctgatggatggaccc |
| | | gcggtgcattagctagttggtgaggtaacggctcaccaaggccacgatgcatagccgacctgagagggtgatcggccacatt |
| | | gggactgagacacggcccaaactcctacgggaggcagcagtagggaatcttcggcaatggacgaaagtctgaccgagcaa |
| | | cgccgcgtgagtgaagaaggttttcggatcgtaaaactctgttgttagagaagaacaaggatgagagtaactgttcatcccttga |
| | | cggtatctaaccagaaagccacggctaactacgtgccagcagccgcggtaatacgtaggtggcaagcgttgtccggatttatt |
| | | gggcgtaaagcgagcgcaggcggtttcttaagtctgatgtgaaagcccccggctcaaccggggagggtcattggaaactgg |
| | | gagacttgagtgcagaagaggagagtggaattccatgtgtagcggtgaaatgcgtagatatatggaggaacaccagtggcga |
| | | aggcggctctctggtctgtaactgacgctgaggctcgaaagcgtggggagcaaacaggattagataccctggtagtccacgc |
| | | cgtaaacgatgagtgctaagtgttggagggtttccgccatca |
| Clostridium_ | SEQ ID | ttatgagagtttgatcctggctcaggacgaacgctggcggcgtgcctaatacatgcaagtcgaacgcttctttttccaccggagc |
| difficile | NO: 2 | ttgctccaccggaaaaagaggagtggcgaacgggtgagtaacacgtgggtaacctgcccatcagaaggggataacacttgg |
| | | aaacaggtgctaataccgtataacaatcgaaaccgcatggttttgatttgaaaggcgctttcgggtgtcgctgatggatggaccc |
| | | gcggtgcattagctagttggtgaggtaacggctcaccaaggccacgatgcatagccgacctgagagggtgatcggccacatt |
| | | gggactgagacacggcccaaactcctacgggaggcagcagtagggaatcttcggcaatggacgaaagtctgaccgagcaa |
| | | cgccgcgtgagtgaagaaggttttcggatcgtaaaactctgttgttagagaagaacaaggatgagagtaactgttcatcccttga |
| | | cggtatctaaccagaaagccacggctaactacgtgccagcagccgcggtaatacgtaggtggcaagcgttgtccggatttatt |
| | | gggcgtaaagcgagcgcaggcggtttcttaagtctgatgtgaaagcccccggctcaaccggggagggtcattggaaactgg |
| | | gagacttgagtgcagaagaggagagtggaattccatgtgtagcggtgaaatgcgtagatatatggaggaacaccagtggcga |
| | | aggcggctctctggtctgtaactgacgctgaggctcgaaagcgtggggagcaaacaggattagataccctggtagtccacgc |
| | | cgtaaacgatgagtgctaagtgttggagggtttccgcccttca |
| Clostridium_ | SEQ ID | ttatgagagtttgatcctggctcaggacgaacgctggcggcgtgcctaatacatgcaagtcgaacgcttctttttccaccggagc |
| difficile | NO: 3 | ttgctccaccggaaaaagaggagtggcgaacgggtgagtaacacgtgggtaacctgcccatcagaaggggataacacttgg |
| | | aaacaggtgctaataccgtataacaatcgaaaccgcatggttttgatttgaaaggcgctttcgggtgtcgctgatggatggaccc |
| | | gcggtgcattagctagttggtgaggtaacggctcaccaaggccacgatgcatagccgacctgagagggtgatcggccacatt |
| | | gggactgagacacggcccaaactcctacgggaggcagcagtagggaatcttcggcaatggacgaaagtctgaccgagcaa |
| | | cgccgcgtgagtgaagaaggttttcggatcgtaaaactctgttgttagagaagaacaaggatgagagtaactgttcatcccttga |
| | | cggtatctaaccagaaagccacggctaactacgtgccagcagccgcggtaatacgtaggtggcaagcgttgtccggatttatt |
| | | gggcgtaaagcgagcgcaggcggtttcttaagtctgatgtgaaagcccccggctcaaccggggagggtcattggaaactgg |
| | | gagacttgagtgcagaagaggagagtggaattccatgtgtagcggtgaaatgcgtagatatatggaggaacaccagtggcga |
| | | aggcggctctctggtctgtaactgacgctgaggctcgaaagcgtggggagcaaacaggattagataccctggtagtccacgc |
| | | cgtaaacgatgagtgctaagtgttggagggtttccgcccttca |
| Fusicateni- | SEQ ID | tcagagagtttgatcctggctcaggatgaacgctggcggcgtgcttaacacatgcaagtcgagcgaagcagttaagttgatttct |
| bacter_ | NO: 4 | tcggattgattcttgactgactgagcggcggacgggtgagtaacgcgtgggtgacctgccccataccgggggataacagctg |
| saccharivorans | | gaaacggctgctaataccgcataagcgcacagagctgcatggctcggtgtgaaaaactccggtggtatgggatgggcccgc |
| | | gtctgattaggcagttggcggggtaacggcccaccaaaccgacgatcagtagccggcctgagagggcgaccggccacattg |
| | | ggactgagacacggcccaaactcctacgggaggcagcagtggggaatattgcacaatgggggaaaccctgatgcagcgac |
| | | gccgcgtgagcgaagaagtatttcggtatgtaaagctctatcagcagggaagataatgacggtacctgactaagaagccccg |
| | | gctaactacgtgccagcagccgcggtaatacgtagggggcaagcgttatccggatttactgggtgtaaagggagcgtagacg |
| | | gcaaggcaagtctgatgtgaaaacccagggcttaaccctgggactgcattggaaactgtctggctcgagtgccggagaggta |
| | | agcggaattcctagtgtagcggtgaaatgcgtagatattaggaagaacaccagtggcgaaggcggcttactggacggtaact |
| | | gacgttgaggctcgaaagcgtggggagcaaacaggattagataccctggtagtccacgccgtaaacgatgaatgctaggtgtt |
| | | ggggagcaaagctcttcggtgccgccgcaaacgcattaagcattccacctggggagtacgttcgcaagaatgaaactcaaag |
| | | gaattgacggggacccgcacaagcggtggagcatgtggtttaattcgaagcaacgcgaagaaccttaccaggtcttgacatcc |
| | | cgatgaccggcccgtaacggggctttctcttcggagcattggagacaggtggtgcatggttgtcgtcagctcgtgtcgtgagat |
| | | gttgggttaagtcccgcaacgagcgcaacccttatcctcagtagccagcaggtaaagctgggcactctgtggagactgccag |
| | | ggataacctggaggaaggtggggatgacgtcaaatcatcatgccccttatgatctgggctacacacgtgctacaatggcgtaa |
| | | acaaagggaggcaaagccgcgaggtggagcaaatcccaaaaataacgtctcagttcggactgcagtctgcaactcgactgc |
| | | acgaagctggaatcgctagtaatcgcgaatcagaatgtcgcggtgaatacgttcccgggtcttgtacacaccgcccgtcacac |
| | | catgggagttggtaacgcccgaagtcagtgacccaaccttttaggagggagctgccgaaggcgggactgataactggggtg |
| | | aagtcgtaacaaggtagccgtatcggaaggtgcggctggatcacctccttt |
| Lachnospira_ | SEQ ID | ttatgagagtttgatcctggctcaggatgaacgctggcggcgtgcttaacacatgcaagtcgaacgaagcatttaagacagatta |
| pectinoschi | NO: 5 | cttcggtttgaagtcttttatgactgagtggcggacgggtgagtaacgcgtgggtaacctgcctcatacagggggatagcagct |
| za | | ggaaacggctggtaataccgcataagcgcacagtaccacatggtacagtgtgaaaaactccggtggtatgagatggacccgc |
| | | gtctgattagcttgttggcggggtaacggcccaccaaggcgacgatcagtagccgacctgagagggtgaccggccacattgg |
| | | gactgagacacggcccagactcctacgggaggcagcagtggggaatattgcacaatggaggaaactctgatgcagcgacg |
| | | ccgcgtgagtgaagaagtagttcgctatgtaaagctctatcagcagggaagatagtgacggtacctgactaagaagctccggc |
| | | taaatacgtgccagcagccgcggtaatacgtatggagcaagcgttatccggatttactgggtgtaaagggagtgtaggtggcc |
| | | aggcaagtcagaagtgaaagcccggggctcaaccccgggactgcttttgaaactgcagggctagagtgcaggaggggcaa |
| | | gtggaattcctagtgtagcggtgaaatgcgtagatattaggaggaacaccagtggcgaaggcggcttgctggactgtaactga |
| | | cactgaggctcgaaagcgtggggagcaaacaggattagataccctggtagtccacgccgtaaacgatgaatactaggtgtcg |
| | | gggcacataagtgctccggtgccgcagcaaacgcaataagtattccacctggggagtacgttcgcaagaatgaaactcaaag |
| | | gaattgacggggacccgcacaagcggtggagcatgtggtttaattcgaagcaacgcgaagaaccttaccaagtcttgacatcc |
| | | tcttgaccggtcagtaatgtgaccttttcttcggaacaagagtgacaggtggtgcatggttgtcgtcagctcgtgtcgtgagatgtt |
| | | gggttaagtcccgcaacgagcgcaacccttatccttagtagccagcagttcggctgggcactctagggagactgccagggat |
| | | aacctggaggaaggtggggatgacgtcaaatcatcatgccccttatgacttgggctacacacgtgctacaatggcgtaaacaa |
| | | agtgaagcgagagcgtgagcttaagcaaatcacaaaaataacgtctcagttcggattgtagtctgcaactcgactacatgaagc |
| | | tggaatcgctagtaatcgcagatcagaatgctgcggtgaatacgttcccgggtcttgtacacaccgcccgtcacaccatggga |
| | | gtcggaaatgcccgaagtcggtgacctaacgtaagaaggagccgccgaaggcaggtctgataactggggtgaagtcgtaac |
| | | aaggtagccgtatcggaaggtgcggctggatcacctccttt |
| Clostridium_ | SEQ ID | tccggtggtatgagatggacccgcgtctgattagctagttggtgaggtaacggcccaccaaggcgacgatcagtagccgacct |
| sp. | NO: 6 | gagagggtgaccggccacattgggactgagacacggcccagactcctacgggaggcagcagtgggggatattgcacaatg |
| | | gaggaaactctgatgcagcgacgccgcgtgagtgaagaagtatttcggtatgtaaagctctatcagcagggaagaaaatgac |
| | | ggtacctgactaagaagccccggctaactacgtgccagcagccgcggtaatacgtagggggcaagcgttatccggatttact |
| | | gggtgtaaagggagcgtagacggcgacgcaagtctgaagtgaaatacccgggctcaacctgggaactgctttggaaactgtg |
| | | ttgctagagtgctggagaggtaagcggaattcctagtgtagcggtgaaatgcgtagatattaggaagaacaccagtggcgaag |
| | | gcggcttactggacagtaactgacgttgaggctcgaaagcgtggggagcaaacaggattagataccctggtagtccacgccg |
| | | taaacgatgaatactaggtgttggtgagcaaagctcatcggtgccgccgcaaacgcaataagtattccacctggggagtacgtt |
| | | cgcaagaatgaaactcaaaggaattgacggggacccgcacaagcggtggagcatgtggtttaattcgaagcaacgcgaaga |
| | | accttaccaaatcttgacatccctctgaaaagcctttaatcgggctcctccttcgggacagaggtgacaggtggtgcatggttgtc |
| | | gtcagctcgtgtcgtgagatgttgggttaagtcccgcaacgagcgcaacccctattgtcagtagccagcaggtaaagctgggc |
| | | actctgatgagactgccagggataacctggaggaaggtggggatgacgtcaaatcatcatgccccttatgatttgggctacaca |
| | | cgtgctacaatggcgtaaacaaagagaagcgagcctgcgagggggagcaaatctcaaaaataacgtctcagttcggattgta |
| | | gtctgcaactcgactacatgaagctggaatcgctagtaatcgcagatcagaatgctgcggtgaatacgttcccgggtcttgtaca |
| | | caccgcccgtcacaccatgggagtcggaaatgcccgaagccagtgacccaagcgaaagcagggagctgtcgaaggcagg |
| | | tctgataactggggtgaagtcgtaacaaggtagccgtatcggaaggtgcggctggatcacctccttt |
| Ruminococcus_ | SEQ ID | tacgagagtttgatcctggctcaggatgaacgctggcggcgtgcttaacacatgcaagtcgaacgaagcacttaagtttgattct |
| sp. | NO: 7 | tcggatgaagacttttgtgactgagtggcggacgggtgagtaacgcgtgggtaacctgcctcatacagggggataacagttag |
| | | aaatggctgctaataccgcataagaccacagcaccgcatggtgcaggggtaaaaactccggtggtatgagatggacccgcgt |
| | | ctgattagctggttggtggggtaacggcctaccaaggcgacgatcagtagccgacctgagagggtgaccggccacattggg |
| | | actgagacacggcccaaactcctacgggaggcagcagtggggaatattgcacaatgggggaaaccctgatgcagcgacgc |
| | | cgcgtgaaggatgaagtatttcggtatgtaaacttctatcagcagggaagaaaatgacggtacctgactaagaagccccggct |
| | | aactacgtgccagcagccgcggtaatacgtagggggcaagcgttatccggatttactgggtgtaaagggagcgtagacggta |
| | | tggcaagtctgatgtgaaaggccagggctcaaccctgggactgcattggaaactgtcgaactagagtgtcggagaggcaagt |
| | | ggaattcctagtgtagcggtgaaatgcgtagatattaggaggaacaccagtggcgaaggcggcttgctggacgatgactgac |
| | | gttgaggctcgaaagcgtggggagcaaacaggattagataccctggtagtccacgccgtaaacgatgactactaggtgtcgg |
| | | gtagcagagctattcggtgccgcagccaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatctccc |
| | | tgaccggcaagtaatgttgcctttccttcgggacagggatgacaggtggtgcatggttgtcgtcagctcgtgtcgtgagatgttg |
| | | ggttaagtcccgcaacgagcgcaacccctatctttagtagccagcggtacggccgggcactctagagagactgccagggata |
| | | acctggaggaaggtggggatgacgtcaaatcatcatgccccttatgagcagggctacacacgtgctacaatggcgtaaacaa |
| | | agggaagcgagcctgcgagggtaagcaaatctcaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gttggtaacgcccgaagtcagtgacccaaccgcaaggagggagctgccgaaggtgggaccgataactggggtgaagtcgt |
| | | aacaaggtagccgtatcggaaggtgcggctggatcacctccttt |
| Clostridium_ | SEQ ID | tttagagagtttgatcctggctcaggatgaacgctggcggcgtgcttaacacatgcaagtcgagcgaagcacttatggagattct |
| sp. | NO: 8 | tcggatgatccatttgtgactgagcggcggacgggtgagtaacgcgtgagtaacctgcctcatacaggggaataacagttaga |
| | | aatgactgctaatgccgcataagcgcacagggccacatggcctggtgtgaaaaactccggtggtatgagatggactcgcgtct |
| | | gattagctagttggtgaggtaacggcccaccaaggcgacgatcagtagccggcctgagagggtgaacggccacattgggac |
| | | tgagacacggcccaaactcctacgggaggcagcagtggggaatattgcacaatgggggaaaccctgatgcagcgacgccg |
| | | cgtgagtgaagaagtatttcggtatgtaaagctctatcagcagggaagaaaatgacggtacctgactaagaagccccggctaa |
| | | ctacgtgccagcagccgcggtaatacgtagggggcaagcgttatccggatttactgggtgtaaagggagcgtagacggctttg |
| | | caagtctgacgtgaaaatccggggctcaaccccggaactgcgttggaaactgtgaggcttgagtgccggagaggtaagcgg |
| | | aattcctagtgtagcggtgaaatgcgtagatattaggaggaacaccagtggcgaaggcggcttactggacggcaactgacgtt |
| | | gaggctcgaaagcgtggggagcaaacaggattagataccctggtagtccacgcggtaaacgatgaatactaggtgttgggg |
| | | gacaaagtccttcggtgccgccgcaaacgcattaagtattccacctggggagtacgttcgcaagaatgaaactcaaaggaatt |
| | | gacggggacccgcacaagcggtggagcatgtggtttaattcgaagcaacgcgaagaaccttaccaagtcttgacatcgattcg |
| | | ccgggactgtaatgagtcctttcccttcggggacgaagaagacaggtggtgcatggttgtcgtcagacgtgtcgtgagatgtt |
| | | gggttaagtcccgcaacgagcgcaacccttatcttcagtagccagcaagtgaagttgggcactctggagagactgccaggga |
| | | caacctggaggaaggtggggatgacgtcaaatcatcatgccccttatgacttgggctacacacgtgctacaatggcgtaaaca |
| | | aagggaagcagccctgtgaaggtgagcaaatcccaaaaataacgtctcagttcggattgtagtctgcaactcgactacatgaa |
| | | gctggaatcgctagtaatcgcgaatcagaatgtcgcggtgaatacgttcccgggtcttgtacacaccgcccgtcacaccatgg |
| | | gagtcggtaacgcccgaagtcagtgacccaaccgaaaggagggagctgccgaaggcgggactggtaactggggtgaagt |
| | | cgtaacaaggtagccgtatcggaaggtgcggctggatcacctccttt |
| Blautia_faecis | SEQ ID | ctccggtggtataagatggacccgcgttggattagctagttggcagggcagcggcctaccaaggcgacgatccatagccggc |
| | NO: 9 | ctgagagggtgaacggccacattgggactgagacacggcccagactcctacgggaggcagcagtggggaatattgcacaat |
| | | gggggaaaccctgatgcagcgacgccgcgtgaaggaagaagtatctcggtatgtaaacttctatcagcagggaagataatga |
| | | cggtacctgactaagaagccccggctaactacgtgccagcagccgcggtaatacgtagggggcgagcgttatccggatttac |
| | | tgggtgtaaagggagcgtagacggcgtatcaagtctgatgtgaaaggcaggggcttaacccctggactgcattggaaactggt |
| | | atgcttgagtgccggaggggtaagcggaattcctagtgtagcggtgaaatgcgtagatattaggaggaacaccagtggcgaa |
| | | ggcggcttactggacggtaactgacgttgaggctcgaaagcgtggggagcaaacaggattagataccctggtagtccacgcc |
| | | gtaaacgatgaatactaggtgtctgggagcacagctcttaggtgccgccgcaaacgcattaagtattccacctggggagtacgt |
| | | tcgcaagaatgaaactcaaaggaattgacggggacccgcacaagcggtggagcatgtggtttaattcgaagcaacgcgaag |
| | | aaccttaccaaatcttgacatccctctgacagagtatgtaatgtacttttccttcgggacaggggagacaggtggtgcatggttgt |
| | | cgtcagctcgtgtcgtgagatgttgggttaagtcccgcaacgagcgcaacccctatccttagtagccagcaagtaatgttgggc |
| | | actctgaggagactgccagggataacctggaggaaggcggggatgacgtcaaatcatcatgccccttatgatttgggctacac |
| | | acgtgctacaatggcgtaaacaaagggaagcgaacctgtgagggtgggcaaatctcaaaaataacgtcccagttcggactgc |
| | | agtctgcaactcgactgcacgaagctggaatcgctagtaatcgcggatcagaatgccgcggtgaatacgttcccgggtcttgta |
| | | cacaccgcccgtcacaccatgggagtcagtaacgcccgaagtcagtgacctaaccgtaaggaaggagctgccgaaggcgg |
| | | gacggatgactggggtgaagtcgtaacaaggtagccgtatcggaaggtgcggctggatcacctccttt |
| Clostridium_ | SEQ ID | taaagagtttgatcctggctcaggacgaacgctgtcggcgcgcctaacacatgcaagtcgaacgaagttgctttgaatgaattct |
| sp. | NO: 10 | tcggaaggaatttgattcaacttagtggcggacgggtgagtaacgcgtgagtaacctgcctttcagagggggataacgtctgg |
| | | aaacggacgctaataccgcataacatattggtttcgcatggagctgatatcaaaggagcaatccgctgaaagatggactcgcgt |
| | | ccaattagctagttggtgaggtaacggcccaccaaggcgacgattggtagccggactgagaggttgaacggccacattggga |
| | | ctgagacacggcccagactcctacgggaggcagcagtggggaatattgcacaatgggggaaaccctgatgcagcgacgcc |
| | | gagtgagggaagaaggttttcggattgtaaacctctgtccttggtgaagataatgacggtaaccaaggaggaagctacggcta |
| | | actacgtgccagcagccgcggtaatacgtaggtagcgagcgttgtccggaattactgggtgtaaagggagcgtaggcgggat |
| | | tgcaagttgaatgttaaatctatgggctcaacccatagccgcgttcaaaactgcagttcttgagtgaagtagaggcaggcggaat |
| | | tcctagtgtagcggtgaaatgcgtaaatattaggaggaacaccagtggcgaaggcggcctgctgggctttaactgacgctgag |
| | | gctcgaaagcgtgggtagcaaacaggattagataccctggtagtccacgctgtaaacgatgattactaggtgtggggggactg |
| | | accccttccgtgccggagttaacacaataagtaatccacctggggagtacgaccgcaaggttgaaactcaaaggaattgacgg |
| | | gggcccgcacaagcagtggagtatgtggtttaattcgaagcaacgcgaagaaccttaccaggtcttgacatccggtgcatagc |
| | | ctagagataggtgaagcccttcggggcaccgagacaggtggtgcatggttgtcgtcagctcgtgtcgtgagatgttgggttaa |
| | | gtcccgcaacgagcgcaacccttacgtttagttgctacgcaagagcactctagacggactgccgttgacaaaacggaggaag |
| | | gtggggatgacgtcaaatcatcatgccccttatgacctgggctacacacgtactacaatggctattaacagagggaagcaagat |
| | | ggtgacatggagcaaacccctaaaaatagtctcagttcggattgcaggctgcaacccgcctgcatgaagccggaattgctagt |
| | | aatcgcggatcagcatgccgcggtgaatacgttcccgggccttgtacacaccgcccgtcacaccatgagagttggcaacacc |
| | | cgaagccgatagtctaaccgcaagggggacgtcgtcgaaggtggggttgatgattggggtgaagtcgtaacaaggtagccgt |
| | | atcggaaggtgcggctggatcacctccttt |
| Clostridium_ | SEQ ID | atgagagtttgatcctggctcaggatgaacgctggcggcgtgcctaacacatgcaagtcgaacggaatttacatgaagcctag |
| sp. | NO: 11 | cgattgtaaatttagtggcggacgggtgagtaacgcgtgggtaacctgccttgtactgggggacaacagttggaaacgactgc |
| | | taataccgcataagcgcacagcttcgcatgaagcagtgtgaaaaactccggtggtacaagatggacccgcgtctgattagctg |
| | | gttggtgaggtaacggcccaccaaggcgacgatcagtagccggcctgagagggtgaacggccacattgggactgagacac |
| | | ggcccaaactcctacgggaggcagcagtggggaatattgcacaatgggggaaaccctgatgcagcaacgccgcgtgagtg |
| | | aagaagtatttcggtatgtaaagctctatcagcaggaaagaaaatgacggtacctgactaagaagccccggctaactacgtgcc |
| | | agcagccgcggtaatacgtagggggcaagcgttatccggatttactgggtgtaaagggagcgtagacggttttgcaagtctga |
| | | agtgaaagcccggggcttaaccccgggactgctttggaaactgtaggactagagtgcaggagaggtaagtggaattcctagt |
| | | gtagcggtgaaatgcgtagatattaggaggaacaccagtggcgaaggcggcttactggactgtaactgacgttgaggctcga |
| | | aagcgtggggagcaaacaggattagataccctggtagtccacgccgtaaacgatgattactaggtgttggtgggtacgaccca |
| | | tcggtgccgcagcaaacgcaataagtaatccacctggggagtacgttcgcaagaatgaaactcaaaggaattgacggggacc |
| | | cgcacaagcggtggagcatgtggtttaattcgaagcaacgcgaagaaccttacctggtcttgacatccctatgaataacgggca |
| | | atgccgttagtacttcggtacataggagacaggtggtgcatggttgtcgtcagctcgtgtcgtgagatgttgggttaagtcccgc |
| | | aacgagcgcaacccttatctttagtagccagcagtaagatgggcactctagagagactgccggggataacccggaggaaggt |
| | | ggggatgacgtcaaatcatcatgccccttatgaccagggctacacacgtgctacaatggcgtaaacaaagagaagcgaagtc |
| | | gtgaggcagagcgaatctcaaaaataacgtctcagttcggattgtagtctgcaactcgactacatgaagctggaatcgctagta |
| | | atcgcagatcagaatgctgcggtgaatacgttcccgggtcttgtacacaccgcccgtcacaccatgggagtcggaaatgcccg |
| | | aagtcggtgacctaaccgaaaggaaggagccgccgaaggcaggtctgataactggggtgaagtcgtaacaaggtagccgta |
| | | tcggaaggtgcggctggatcacctccttt |
| Clostridium_ | SEQ ID | atgagagtttgatcctggctcaggatgaacgctggcggcgtgcctaacacatgcaagtcgaacgaggtattttgattgaagtttt |
| sp. | NO: 12 | cggatggatttcaagataccgagtggcggacgggtgagtaacgcgtgggtaacctgcctcatacagggggataacggttaga |
| | | aatgactgctaataccgcataagcgcacagtaccgcatggtacggtgtgaaaaactccggtggtatgagatggacccgcgtct |
| | | gattagctagttggtggggtaacggcccaccaaggcgacgatcagtagccgacctgagagggtgaccggccacattgggac |
| | | tgagacacggcccagactcctacgggaggcagcagtgggggatattgcacaatggaggaaactctgatgcagcgacgccg |
| | | cgtgagtgaagaagtatttcggtatgtaaagctctatcagcagggaagaaaatgacggtacctgactaagaagccccggctaa |
| | | ctacgtgccagcagccgcggtaatacgtagggggcaagcgttatccggatttactgggtgtaaagggagcgtagacggcga |
| | | cgcaagtctgaagtgaaatacccgggctcaacctgggaactgctttggaaactgtgttgctagagtgctggagaggtaagcgg |
| | | aattcctagtgtagcggtgaaatgcgtagatattaggaagaacaccagtggcgaaggcggcttactggacagtaactgacgtt |
| | | gaggctcgaaagcgtggggagcaaacaggattagataccctggtagtccacgccgtaaacgatgaatactaggtgttggtga |
| | | gcaaagctcatcggtgccgccgcaaacgcaataagtattccacctggggagtacgttcgcaagaatgaaactcaaaggaattg |
| | | acggggacccgcacaagcggtggagcatgtggtttaattcgaagcaacgcgaagaaccttaccaaatcttgacatccctctga |
| | | aannnnnnnnnnnnnnnnnnnnnnnnnnnnnnnnnnnnnnnnnnnnnnncctccttcgggacagaggtgacagg |
| | | tggtgcatggttgtcgtcagctcgtgtcgtgagatgttgggttaagtcccgcaacgagcgcaacccctattgtcagtagccagca |
| | | ggtaaagctgggcactctgatgagactgccagggataacctggaggaaggtggggatgacgtcaaatcatcatgccccttatg |
| | | atttgggctacacacgtgctacaatggcgtaaacaaagagaagcgagcctgcgagggggagcaaatctcaaaaataacgtct |
| | | cagttcggattgtagtctgcaactcgactacatgaagctggaatcgctagtaatcgcagatcagaatgctgcggtgaatacgttc |
| | | ccgggtcttgtacacaccgcccgtcacaccatgggagtcggaaatgcccgaagccagtgacccaagcgaaagcagggagc |
| | | tgtcgaaggcaggtctgataactggggtgaagtcgtaacaaggtagccgtatcggaaggtgcggctggatcacctccttt |
| Flavonifractor_ | SEQ ID | tattgagagtttgatcctggctcaggatgaacgctggcggcgtgcttaacacatgcaagtcgaacggagtgctcatgacagag |
| sp. | NO: 13 | gattcgtccaatggagtgagttacttagtggcggacgggtgagtaacgcgtgagtaacctgccttggagtggggaataacagg |
| | | tggaaacatctgctaataccgcatgatgcagttgggtcgcatggctctgactgccaaagatttatcgctctgagatggactcgcg |
| | | tctgattagctggttggcggggtaacggcccaccaaggcgacgatcagtagccggactgagaggttggccggccacattgg |
| | | gactgagacacggcccagactcctacgggaggcagcagtggggaatattgggcaatgggcgcaagcctgacccagcaac |
| | | gccgcgtgaaggaagaaggctttcgggttgtaaacttcttttctcagggacgaagcaagtgacggtacctgaggaataagcca |
| | | cggctaactacgtgccagcagccgcggtaatacgtaggtggcgagcgttatccggatttactgggtgtaaagggcgtgtaggc |
| | | gggactgcaagtcagatgtgaaaaccatgggctcaacctgtggcctgcatttgaaactgtagttcttgagtactggagaggcag |
| | | acggaattcctagtgtagcggtgaaatgcgtagatattaggaggaacaccagtggcgaaggcggtctgctggacagcaactg |
| | | acgctgaggcgcgaaagcgtggggagcaaacaggattagataccctggtagtccacgctgtaaacgatggatactaggtgtg |
| | | gggggtctgaccccctccgtgccgcagttaacacaataagtatcccacctggggagtacgatcgcaaggttgaaactcaaag |
| | | gaattgacgggggcccgcacaagcggtggagtatgtggtttaattcgaagcaacgcgaagaaccttaccagggcttgacatc |
| | | ccggtgaccggtgtagagatacaccttcttcttcggaagcgccggtgacaggtggtgcatggttgtcgtcagctcgtgtcgtga |
| | | gatgttgggttaagtcccgcaacgagcgcaacccttattgttagttgctacgcaagagcactctagcgagactgccgttgacaa |
| | | aacggaggaaggtggggacgacgtcaaatcatcatgccccttatgtcctgggccacacacgtactacaatggtggtcaacag |
| | | agggaagcaagaccgcgaggtggagcaaacccctaaaagccatcccagttcggattgcaggctgcaactcgcctgtatgaa |
| | | gttggaatcgctagtaatcgcggatcagcatgccgcggtgaatacgttcccgggccttgtacacaccgcccgtcacaccatga |
| | | gagtcgggaacacccgaagtccgtagcctaaccgcaaggggggcgcggccgaaggtgggttcgataattggggtgaagtc |
| | | gtaacaaggtagccgtatcggaaggtgcggctggatcacctccttt |
| Emergencia_ | SEQ ID | attaagagtttgatcctggctcaggatgaacgctggcggcgtgcctaacacatgcaagtcgagcgagaagccattgaccgata |
| timonensis | NO: 14 | cttcggtaaagggatatggcggaaagcggcggacgggtgagtaacgcgtaggcaacctgccccttacagagggatagccat |
| | | tggaaacgatgattaagacctcataacgcctccctcccacatgagggggaggccaaagattcatcggtaagggatgggcctg |
| | | cgtctgattagcttgttggcggggtaacggcccaccaaggcgacgatcagtagccgacctgagagggtgatcggccacattg |
| | | gaactgagacacggtccaaactcctacgggaggcagcagtggggaatattgcacaatgggcggaagcctgatgcagcaac |
| | | gccgcgtgagggatgaaggccttcgggtcgtaaacctctgtccttggggaagaaacaaatgacggtacccatggaggaagc |
| | | cccggctaactacgtgccagcagccgcggtaatacgtagggggcgagcgttatccggaattattgggcgtaaagagtgcgta |
| | | ggtggttacctaagcgcagggtctaaggcaatggctcaaccattgttcgccctgcgaactgggctacttgagtgcaggagagg |
| | | aaagcggaattcctagtgtagcggtgaaatgcgtagatattaggaggaacaccagtggcgaaggcggctttctggactgttact |
| | | gacactgaggcacgaaagcgtggggagcaaacaggattagataccctggtagtccacgccgtaaacgatgagcactaggtg |
| | | tcggggccgcaaggcttcggtgccgcagttaacgcattaagtgctccgcctggggagtacgcacgcaagtgtgaaactcaaa |
| | | ggaattgacggggacccgcacaagcagcggagcatgtggtttaattcgaagcaacgcgaagaaccttaccaggacttgacat |
| | | ccccctgacagatccttaaccggatccttcttcggacaggggagacaggtggtgcatggttgtcgtcagctcgtgtcgtgagat |
| | | gttgggttaagtcccgcaacgagcgcaacccttgccattagttgccatcattcagttgggcactctaatgggactgccggggac |
| | | aactcggaggaaggtggggatgacgtcaaatcatcatgccccttatgttctgggctacacacgtgctacaatggccggtacag |
| | | caggaagcgatcccgcgagggggagcaaatcccaaaaaccggtcccagttcggactgcaggctgcaacccgcctgcacga |
| | | agccggagttgctagtaatcgtggatcagaatgccacggtgaatgcgttcccgggtcttgtacacaccgcccgtcacaccatg |
| | | gaagttgggggtgcccgaagccggcagggagatatgctgtctaaggcaaaaccaatgactggggtgaagtcgtaacaaggt |
| | | agccgtatcggaaggtgcggctggatcacctccttt |
| Clostridium_ | SEQ ID | atgagagtttgatcctggctcaggatgaacgctggcggcgtgcctaacacatgcaagtcgaacgaggtaaatgagatgaagtt |
| sp. | NO: 15 | ttcggatggattcttatttaccgagtggcggacgggtgagtaacgcgtgggtaacctgcctcatacagggggataacgattgga |
| | | aacgattgctaataccgcataagcgcacagtaccacatggtacagtgtgaaaaactccggtggtatgagatggacccgcgtct |
| | | gattagctagttggtgaggtaacggcccaccaaggcaacgatcagtagccgacctgagagggtgaccggccacattgggac |
| | | tgagacacggcccagactcctacgggaggcagcagtgggggatattgcacaatggaggaaactctgatgcagcgacgccg |
| | | cgtgagtgaagaagtatttcggtatgtaaagctctatcagcagggaagaaaatgacggtacctgactaagaagccccggctaa |
| | | ctacgtgccagcagccgcggtaatacgtagggggcaagcgttatccggatttactgggtgtaaagggagcgtagacggcgat |
| | | gcaagtctgaagtgaaatacccgggctcaacctgggaactgctttggaaactgtatggctagagtgctggagaggtaagcgg |
| | | aattcctagtgtagcggtgaaatgcgtagatattaggaagaacaccagtggcgaaggcggcttactggacagtaactgacgttc |
| | | aggctcgaaagcgtggggagcaaacaggattagataccctggtagtccacgccgtaaacgatgaatactaggtgtcggggg |
| | | acaaagtctttcggtgccgccgcaaacgcaataagtattccacctggggagtacgttcgcaagaatgaaactcaaaggaattga |
| | | cggggacccgcacaagcggtggagcatgtggtttaattcgaagcaacgcgaagaaccttaccaaatcttgacatccctctgaa |
| | | aagcctttaatcgggctcctccttcgggacagaggtgacaggtggtgcatggttgtcgtcagctcgtgtcgtgagatgttgggtt |
| | | aagtcccgcaacgagcgcaacccctattgtcagtagccagcaggtaaagctgggcactctgatgagactgccagggataacc |
| | | tggaggaaggtggggatgacgtcaaatcatcatgccccttatgatttgggctacacacgtgctacaatggcgtaaacaaagag |
| | | aggcgaagctgtgaggcagagcaaatctcaaaaataacgtctcagttcggattgtagtctgcaactcgactacatgaagctgga |
| | | atcgctagtaatcgcggatcagaatgccgcggtgaatacgttcccgggtcttgtacacaccgcccgtcacaccatgggagtcg |
| | | gaaatgcccgaagccagtgacccaagcgaaagcagggagctgtcgaaggcaggtctgataactggggtgaagtcgtaaca |
| | | aggtagccgtatcggaaggtgcggctggatcacctccttt |
| Clostridium_ | SEQ ID | nccggatttactgggtgtaaagggtgcgtaggcggaactgcaagtcagtggtgaaatctgagggctcaaccctcaaactgcca |
| sp. | NO: 16 | ttgaaactgtggttcttgagtgaagtagaggtaggcggaattcccggtgtagcggtggaatgcgtagagatcgggaggaacac |
| | | cagtggcgaaggcggcctactgggctttaactgacgctgaggcacgaaagcatgggtagcaaacaggattagataccctggt |
| | | agtccatgccgtaaacgatgattactaggtgtggggggtctgaccccctccgtgccggagttaacacaataagtaatccacctg |
| | | gggagtacggccgcaaggttgaaactcaaaggaattgacgggggcccgcacaagcagtggagtatgtggtttaattcgacgc |
| | | aacgcgaagaaccttaccaggtcttgacatccaactaacgaggtagagatacgtcaggtgcccttcggggaaagttgagaca |
| | | ggtggtgcatggttgtcgtcagctcgtgtcgtgagatgttgggttaagtcccgcaacgagcgcaacccttgtgattagttgctac |
| | | gcaagagcactctaatcagactgccgttgacaaaacggaggaaggtggggatgacgtcaaatcatcatgccctttatgacctg |
| | | ggctacacacgtactacaatggccgtaaacagagagaggcgacaccgcgaggtgaagcgaaaccctaaaaacggtctcagt |
| | | tcggattgtaggctgcaacccgcctacatgaagttggaattgctagtaatcgcggatcagcatgccgcggtgaatacgttcccg |
| | | ggccttgtacacaccgcccgtcacaccatgggagccggtaatacccgaagtcagtagtctaaccgcaaggggggcgctgcc |
| | | gaaggtaggattggcgactggggtgaagtcgtaacaaggtagccgtatcggaaggtgcggctggatcacctccttt |
| Clostridium_ | SEQ ID | acggcccagactcctacgggaggcagcagtggggaatattgcgcaatgggggaaaccctgacgcagcaacgccgcgtgat |
| sp. | NO: 17 | tgaagaaggccttcgggttgtaaagatctttaattagggacgaaataaatgacggtacctgaagaataagctccggctaactac |
| | | gtgccagcagccgcggtaatacgtagggagcaagcgttatccggatttactgggtgtaaagggcgcgcaggcgggccggta |
| | | agttggaagtgaaatctatgggcttaacccataaactgctttcaaaactgctggtcttgagtgatggagaggcaggcggaattcc |
| | | gtgtgtagcggtgaaatgcgtagatatacggaggaacaccagtggcgaaggcggcctgctggacattaactgacgctgagg |
| | | cgcgaaagcgtggggagcaaacaggattagataccctggtagtccacgccgtaaacgatggatactaggtgtgggaggtatt |
| | | gaccccttccgtgccgcagttaacacaataagtatcccacctggggagtacggccgcaaggttgaaactcaaaggaattgacg |
| | | ggggcccgcacaagcagtggagtatgtggtttaattcgaagcaacgcgaagaaccttaccaggtcttgacatcccgatgaccg |
| | | ncttttcttcggaacatcggtgacaggtggtgcatggttgtcgtcagctcgtgtcgtgagatgttgggttaagtcccgcaacgag |
| | | cgcaacccttacggttagttgatacgcaagatcactctagccggactgccgttgacaaaacggaggaaggtggggacgacgt |
| | | caaatcatcatgccccttatgacctgggctacacacgtactacaatggcagtcatacagagggaagcaaaaccgcgaggtgg |
| | | agcaaatccctaaaagctgtcccagttcagattgcaggctgcaacccgcctgcatgaagtcggaattgctagtaatcgcggatc |
| | | agcatgccgcggtgaatacgttcccgggccttgtacacaccgcccgtcacaccatgagagccgtcaatacccgaagtccgta |
| | | gcctaac |
| Clostridium_ | SEQ ID | tttagagagtttgatcctggctcaggatgaacgctggcggcgtgcctaacacatgcaagtcgaacggggttattttggaaatctc |
| sp. | NO: 18 | ttcggagatggaattcttaacctagtggcggacgggtgagtaacgcgtgagcaatctgcctttaggagggggataacagtcgg |
| | | aaacggctgctaataccgcataatacgtttgggaggcatctcttgaacgtcaaagattttatcgcctttagatgagctcgcgtctg |
| | | attagctggttggcggggtaacggcccaccaaggcgacgatcagtagccggactgagaggttgaacggccacattgggact |
| | | gagacacggcccagactcctacgggaggcagcagtggggaatattgcgcaatgggggaaaccctgacgcagcaacgccg |
| | | cgtgattgaagaaggccttcgggttgtaaagatctttaatcagggacgaaaaatgacggtacctgaagaataagctccggctaa |
| | | ctacgtgccagcagccgcggtaatacgtagggagcaagcgttatccggatttactgggtgtaaagggcgcgcaggcgggcc |
| | | ggcaagttgggagtgaaatcccggggcttaaccccggaactgctttcaaaactgctggtcttgagtgatggagaggcaggcg |
| | | gaattccgtgtgtagcggtgaaatgcgtagatatacggaggaacaccagtggcgaaggcggcctgctggacattaactgacg |
| | | ctgaggcgcgaaagcgtggggagcaaacaggattagataccctggtagtccacgccgtaaacgatggatactaggtgtggg |
| | | aggtattgaccccttccgtgccgcagttaacacaataagtatcccacctggggagtacggccgcaaggttgaaactcaaagga |
| | | attgacgggggcccgcacaagcagtggagtatgtggtttaattcgaagcaacgcgaagaaccttaccaggtcttgacatcccg |
| | | atgaccggcgtagagatacgccctctcttcggagcatcggtgacaggtggtgcatggttgtcgtcagctcgtgtcgtgagatgtt |
| | | gggttaagtcccgcaacgagcgcaacccttacggttagttgatacgcaagatcactctagccggactgccgttgacaaaacgg |
| | | aggaaggtggggacgacgtcaaatcatcatgccccttatgacctgggctacacacgtactacaatggcagtcatacagaggg |
| | | aagcaataccgcgaggtggagcaaatccctaaaagctgtcccagttcagattgcaggctgcaacccgcctgcatgaagtcgg |
| | | aattgctagtaatcgcggatcagcatgccgcggtgaatacgttcccgggccttgtacacaccgcccgtcacaccatgagagcc |
| | | gtcaatacccgaagtccgtagcctaaccgcaaggggggcgcggccgaaggtaggggtggtaattagggtgaagtcgtaaca |
| | | aggtagccgtatcggaaggtgcggctggatcacctccttt |
| Flavonifractor_ | SEQ ID | tattgagagtttgatcctggctcaggatgaacgctggcggcgtgcttaacacatgcaagtcgaacggagtgcccatgacagag |
| sp. | NO: 19 | gattcgtccaatggattgggttacttagtggcggacgggtgagtaacgcgtgaggaacctgcctcggagtggggaataacaca |
| | | acgaaagctgtgctaataccgcatgatacagttgggtcgcatgactctgactgtcaaagatttatcgctctgagatggcctcgcg |
| | | tctgattagctagttggtggggtaacggcccaccaaggcgacgatcagtagccggactgagaggttgaccggccacattggg |
| | | actgagacacggcccagactcctacgggaggcagcagtggggaatattgggcaatgggcgcaagcctgacccagcaacgc |
| | | cgcgtgaaggaagaaggctttcgggttgtaaacttcttttctcagggacgaagcaagtgacggtacctgaggaataagccacg |
| | | gctaactacgtgccagcagccgcggtaatacgtaggtggcaagcgttatccggatttactgggtgtaaagggcgtgtaggcgg |
| | | gatcgcaagtcagatgtgaaaactggaggctcaacctccagcctgcatttgaaactgtggttcttgagtactggagaggcagac |
| | | ggaattcctagtgtagcggtgaaatgcgtagatattaggaggaacaccagtggcgaaggcggtctgctggacagcaactgac |
| | | gctgaggcgcgaaagcgtggggagcaaacaggattagataccctggtagtccacgctgtaaacgatggatactaggtgtgg |
| | | ggggtctgaccccctccgtgccgcagttaacacaataagtatcccacctggggagtacgatcgcaaggttgaaactcaaagga |
| | | attgacgggggcccgcacaagcggtggagtatgtggtttaattcgaagcaacgcgaagaaccttaccagggcttgacatccc |
| | | gaggcccggcctagagataggcctttctcttcggagacctcggtgacaggtggtgcatggttgtcgtcagctcgtgtcgtgaga |
| | | tgttgggttaagtcccgcaacgagcgcaacccctattgttagttgctacgcaagagcactctagcgagactgccgttgacaaaa |
| | | cggaggaaggtggggacgacgtcaaatcatcatgccccttatgtcctgggccacacacgtactacaatggtggttaacagag |
| | | ggaggcaagaccgtgaggtggagcaaatccctaaaagccatcccagttcggattgcaggctgcaacccgcctgcatgaagtt |
| | | ggaatcgctagtaatcgcggatcagcatgccgcggtgaatacgttcccgggccttgtacacaccgcccgtcacaccatgaga |
| | | gtcgggaacacccgaagtccgtagcctaacagcaatgggggcgcggccgaaggtgggttcgataattggggtgaagtcgta |
| | | acaaggtagccgtatcggaaggtgcggctggatcacctccttt |
| Intestinimonas_ | SEQ ID | tattgagagtttgatcctggctcaggatgaacgctggcggcgtgcttaacacatgcaagtcgaacggaacgccaaggaaaga |
| massiliensis | NO: 20 | gttttcggacaatggaattggctgtttagtggcggacgggtgagtaacgcgtgagtaacctgccttggagtggggaataacaca |
| | | gtgaaaactgtgctaataccgcatgacatattggtgtcgcatggcactgatatcaaagatttatcgctctgagatggactcgcgtc |
| | | tgattagatagttggcggggtaacggcccaccaagtcgacgatcagtagccggactgagaggttggccggccacattgggac |
| | | tgagacacggcccagactcctacgggaggcagcagtggggaatattgggcaatgggcgcaagcctgacccagcaacgcc |
| | | gcgtgaaggaagaaggctttcgggttgtaaacttcttttaacagggacgaagtaagtgacggtacctgttgaataagccacggc |
| | | taactacgtgccagcagccgcggtaatacgtaggtggcaagcgttatccggatttactgggtgtaaagggcgtgtaggcggga |
| | | ctgcaagtcagatgtgaaaactatgggctcaacccatagcctgcatttgaaactgtagttcttgagtgtcggagaggcaatcgga |
| | | attccgtgtgtagcggtgaaatgcgtagatatacggaggaacaccagtggcgaaggcggattgctggacgataactgacgct |
| | | gaggcgcgaaagcgtggggagcaaacaggattagataccctggtagtccacgccgtaaacgatggatactaggtgtggggg |
| | | gtctgaccccctccgtgccgcagctaacgcaataagtatcccacctggggagtacgatcgcaaggttgaaactcaaaggaatt |
| | | gacgggggcccgcacaagcggtggagtatgtggtttaattcgaagcaacgcgaagaaccttaccagggcttgacatcctact |
| | | aacgaaccagagatggattaggtgcccttcggggaaagtagagacaggtggtgcatggttgtcgtcagctcgtgtcgtgagat |
| | | gttgggttaagtcccgcaacgagcgcaacccttattgttagttgctacgcaagagcactctagcgagactgccgttgacaaaac |
| | | ggaggaaggtggggacgacgtcaaatcatcatgccccttatgtcctgggccacacacgtactacaatggcggttaacagagg |
| | | gaggcaaagccgcgaggcagagcaaacccctaaaagccgtcccagttcggattgcaggctgaaacccgcctgtatgaagtc |
| | | ggaatcgctagtaatcgcggatcagcatgccgcggtgaatacgttcccgggccttgtacacaccgcccgtcacaccatgaga |
| | | gtcgggaacacccgaagtccgtagcctaactgcaaagggggcgcggccgaaggtgggttcgataattggggtgaagtcgta |
| | | acaaggtagccgtatcggaaggtgcggctggatcacctccttt |
| Clostridium_ | SEQ ID | atggagagtttgatcctggctcaggatgaacgctggcggcgtgcctaatacatgcaagtcgaacgcttcacttcggtgaagagt |
| sp. | NO: 21 | ggcgaacgggtgagtaatacataagtaacctggcatctacagggggataactgatggaaacgtcagctaagaccgcataggt |
| | | gtagagatcgcatgaactctatatgaaaagtgctacgggactggtagatgatggacttatggcgcattagctggttggtagggta |
| | | acggcctaccaaggcgacgatgcgtagccgacctgagagggtgaccggccacactgggactgagacacggcccagactc |
| | | ctacgggaggcagcagtagggaattttcggcaatgggggaaaccctgaccgagcaacgccgcgtgaaggaagaagtaattc |
| | | gttatgtaaacttctgtcatagaggaagaacggtggatatagggaatgatatccaagtgacggtactctataagaaagccacgg |
| | | ctaactacgtgccagcagccgcggtaatacgtaggtggcgagcgttatccggaattattgggcgtaaagagggagcaggcg |
| | | gcactaagggtagtggtgaaagatcgaagcttaacttcggtaagccatggaaaccgtagagctagagtgtgtgagaggatcg |
| | | tggaattccatgtgtagcggtgaaatgcgtagatatatggaggaacaccagtggcgaaggcgacgatctggcgcataactgac |
| | | gctcagtcccgaaagcgtggggagcaaataggattagataccctagtagtccacgccgtaaacgatgagtactaagtgttggg |
| | | tgtcaaagctcagtgctgcagttaacgcaataagtactccgcctgagtagtacgttcgcaagaatgaaactcaaaggaattgac |
| | | gggggcccgcacaagcggtggagcatgtggtttaattcgaagcaacgcgaagaaccttaccaggtcttgacatcgatctaaa |
| | | ggctccagagatggagagatagctatagagaagacaggtggtgcatggttgtcgtcagctcgtgtcgtgagatgttgggttaa |
| | | gtcccgcaacgagcgcaacccctgttgccagttgccagcattaagttggggactctggcgagactgccggtgacaagccgga |
| | | ggaaggcggggatgacgtcaaatcatcatgccccttatgacctgggctacacacgtgctacaatggacagagcagagggaa |
| | | gcgaagccgcgaggtggagcgaaacccataaaactgttctcagttcggactgcagtctgcaactcgactgcacgaagatgga |
| | | atcgctagtaatcgcgaatcagcatgtcgcggtgaatacgttctcgggccttgtacacaccgcccgtcacaccatgagagtcgg |
| | | taacacccgaagccggtggcctaaccgcaaggaaggagctgtctaaggtgggactgatgattggggtgaagtcgtaacaag |
| | | gtatccctacgggaacgtggggatggatcacctccttt |
| Blautia_faecis | SEQ ID | tattgagagtttgatcctggctcaggacgaacgctggcggcatgcctaacacatgcaagtcgagcgagttgatctcttcggagt |
| | NO: 22 | gaagctagcggcggacgggtgagtaacacgtgggcaacctgcctcatagaggggaatagcctcccgaaagggagattaata |
| | | ccgcataagattgtagcttcgcatgaagnagcaattaaaggagcaatccgctatgagatgggcccgcggcgcattagctagtt |
| | | ggtgaggtaacggctcaccaaggcgacgatgcgtagccgacctgagagggtgatcggccacattgggactgagacacggc |
| | | ccagactcctacgggaggcagcagtggggaatattgcacaatgggggaaaccctgatgcagcaatgccgcgtgagtgatga |
| | | cggccttcgggagtaaagctctgtcttcggggacgataatgacggtacccgaggaggaagccacggctaactacgtgccag |
| | | cagccgcggtaatacgtaggtggcaagcgttgtccggatttactgggcgtaaagggagcgtaggcggatttttaagtgagatgt |
| | | gaaatacccgggcttaacttgggtgctgcatttcaaactggaagtctagagtgcaggagaggagaagggaattcctagtgtag |
| | | cggtgaaatgcgtagagattaggaagaacaccagtggcgaaggcgcttctctggactgtaactgacgctgaggctcgaaagc |
| | | gtggggagcaaacaggattagataccctggtagtccacgccgtaaacgatgaatactaggtgtaggggttgtcatgacctctgt |
| | | gccgccgctaacgcattaagtattccgcctggggagtacggtcgcaagattaaaactcaaaggaattgacgggggcccgcac |
| | | aagcagcggagcatgtggtttaattcgaagcaacgcgaagaaccttacctagacttgacatctcctgcattacccttaatcgggg |
| | | aagtcgcttcggcgacaggaagacaggtggtgcatggttgtcgtcagctcgtgtcgtgagatgttgggttaagtcccgcaacg |
| | | agcgcaacccttattgttagttgctaccatttagttgagcactctagcgagactgcccgggttaaccgggaggaaggtggggat |
| | | gacgtcaaatcatcatgccccttatgtctagggctacacacgtgctacaatggcaagtacaaagagaagcaagaccgcgaggt |
| | | ggagcaaaactcaaaaacttgtctcagttcggattgtaggctgaaactcgcctacatgaagctggagttgctagtaatcgcgaat |
| | | cagcatgtcgcggtgaatacgttcccgggccttgtacacaccgcccgtcacaccatgagagttggcaatacccaaagtgcgtg |
| | | atctaactcgtaagagaggaagcgccctaaggtagggtcagcgattggggtgaagtcgtaacaaggtagccgtaggagaac |
| | | ctgcggctggatcacctccttt |
| Clostridium_ | SEQ ID | agtgagagtttgatcctggctcaggacgaacgctggcggcgtgcctaacacatgcaagtcgagcgagtgaatctccttcggga |
| disporicum | NO: 23 | gtgaagctagcggcggacgggtgagtaacacgtgggcaacctgcctcatagaggggaatagccttccgaaaggaagattaa |
| | | taccgcataagattgtagcttcgcatgaagtagcaattaaaggagcaatccgctatgagatgggcccgcggcgcattagctagt |
| | | tggtgaggtaacggctcaccaaggcgacgatgcgtagccgacctgagagggtgatcggccacattgggactgagacacgg |
| | | cccagactcctacgggaggcagcagtggggaatattgcacaatgggggaaaccctgatgcagcaacgccgcgtgagtgatg |
| | | acggccttcgggttgtaaagctctgtcttcagggacgataatgacggtacctgaggaggaagccacggctaactacgtgccag |
| | | cagccgcggtaatacgtaggtggcgagcgttgtccggatttactgggcgtaaagggagcgtaggcggacttttaagtgagatg |
| | | tgaaatacccgggctcaacttgggtgctgcatttcaaactggaagtctagagtgcaggagaggagaatggaattcctagtgtag |
| | | cggtgaaatgcgtagagattaggaagaacaccagtggcgaaggcgattctctggactgtaactgacgctgaggctcgaaagc |
| | | gtggggagcaaacaggattagataccctggtagtccacgccgtaaacgatgaatactaggtgtaggggttgtcatgacctctgt |
| | | gccgccgctaacgcattaagtattccgcctggggagtacggtcgcaagattaaaactcaaaggaattgacgggggcccgcac |
| | | aagcauggagcatgtggtttaattcgaagcaacgcgaagaaccttacctagacttgacatctcctgcattacccttaatcgggg |
| | | aagttccttcgggaacaggaagacaggtggtgcatggttgtcgtcagctcgtgtcgtgagatgttgggttaagtcccgcaacga |
| | | gcgcaacccttattgttagttgctaccatttagttgagcactctagcgagactgcccgggttaaccgggaggaaggtggggatg |
| | | acgtcaaatcatcatgccccttatgtctagggctacacacgtgctacaatggcaagtacaaagagaagcaagaccgcgaggtg |
| | | gagcaaaactcaaaaacttgtctcagttcggattgtaggctgaaactcgcctacatgaagctggagttgctagtaatcgcgaatc |
| | | agcatgtcgcggtgaatacgttcccgggccttgtacacaccgcccgtcacaccatgagagttggcaatacccaaagtgcgtga |
| | | tctgactcgcaagagaggaagcgccctaaggtagggtcagcgattggggtgaagtcgtaacaaggtagccgtaggagaacc |
| | | tgcggctggatcacctccttt |
| Anaerostipes_ | SEQ ID | atgagagttcgatcctggctcaggataaacgctggcggcgtgcttaacacatgcaagtcgaacgaagtttttctttcgggagga |
| sp. | NO: 24 | acttagtggcggacgggtgagtaacgcgtgggtaacctgccctgtacagggggacaacagctggaaacggctgctaatacc |
| | | gcataagcccttagcactgcatggtgcatagggaaaaggagcaatccggtacaggatggacccgcgtctgattagccagttg |
| | | gcagggtaacggcctaccaaagcgacgatcagtagccgatctgagaggatgtacggccacattgggactgagacacggcc |
| | | cagactcctacgggaggcagcagtggggaatattgcacaatggaggaaactctgatgcagcgacgccgcgtgagtgaagaa |
| | | gtatttcggtatgtaaagctctatcagcagggaagaaaatgacggtacctgactaagaagcaccggctaaatacgtgccagca |
| | | gccgcggtaatacgtatggtgcaagcgttatccggatttactgggtgtaaagggagcgtaggtggcaaggcaagccagaagt |
| | | gaaaacccggggctcaaccgcgggattgcttttggaactgtcatgctagagtgcaggaggggtgagcggaattcctagtgtag |
| | | cggtgaaatgcgtagatattaggaggaacaccggaggcgaaggcggctcactggactgtaactgacactgaggctcgaaag |
| | | cgtggggagcaaacaggattagataccctggtagtccacgcggtaaacgatgaatactagatgtcgggtagcaaagctactc |
| | | ggtgtcgtcgcaaacgcaataagtattccacctggggagtacgttcgcaagaatgaaactcaaaggaattgacggggacccg |
| | | cacaagcggtggagcatgtggtttaattcgaagcaacgcgaagaaccttacctgctcttgacatcccattcgatagagggtaat |
| | | gcctctagcccttcgggggaatggagacaggtggtgcatggttgtcgtcagctcgtgtcgtgagatgttgggttaagtcccgca |
| | | acgagcgcaacccttattgtcagtagccagcaggtgaagctgggcactctgatgagactgccggggataacccggaggaag |
| | | gtggggatgacgtcaaatcatcatgccccttatgagcagggctacacacgtgctacaatggcgtaaacagagggaagcgaag |
| | | gagtgatccggagcaaatctcaaaaataacgtctcagttcggattgtagtctgcaactcgactacatgaagctggaatcgctagt |
| | | aatcgcagatcagaatgctgcggtgaatacgttcccgggtcttgtacacaccgcccgtcacaccatgggagtcggtaatgccc |
| | | gaagtcagtgactcaaccgaaaggagagagctgccgaaggcaggactggtaactggggtgaagtcgtaacaaggtagccg |
| | | tatcggaaggtgcggctggatcacctccttt |
| Ruminococcus_ | SEQ ID | tttagagagtttgatcctggctcaggacgaacgctggcggcgtgcctaacacatgcaagtcgaacggagcttgaggggcttgc |
| bicirculans | NO: 25 | ccttcaagcttagtggcggacgggtgagtaacgcgtgagtaacctgcctctcagagggggataacgttctgaaaaggacgcta |
| | | ataccgcatgacatcccggggccgcatggccctgggatcaaaggagcaatccgctgagagatggactcgcgtccgattagct |
| | | agttggtgagataaaggcccaccaaggcgacgatcggtagccggactgagaggttgaacggccacattgggactgagacac |
| | | ggcccagactcctacgggaggcagcagtgggggatattgcacaatggagggaactctgatgcagcaacgccgcgtgaggg |
| | | aagaaggtcttcggattgtaaacctttgtcctcagggaagataatgacggtacctgaggaggaagctccggctaactacgtgcc |
| | | agcagccgcggtaatacgtagggagcaagcgttgtccggatttactgggtgtaaagggtgcgtaggcggaactgcaagtcag |
| | | tggtgaaatctgagggctcaaccctcaaactgccattgaaactgtggttcttgagtgaagtagaggtaggcggaattcccggtgt |
| | | agcggtggaatgcgtagagatcgggaggaacaccagtggcgaaggcggcctactgggctttaactgacgctgaggcacga |
| | | aagcatgggtagcaaacaggattagataccctggtagtccatgccgtaaacgatgattactaggtgtggggggtctgaccccct |
| | | ccgtgccggagttaacacaataagtaatccacctggggagtacggccgcaaggttgaaactcaaaggaattgacgggggcc |
| | | cgcacaagcagtggagtatgtggtttaattcgacgcaacgcgaagaaccttaccaggtcttgacatccaactaacgaggtaga |
| | | gatacgtcaggtgcccttcggggaaagttgagacaggtggtgcatggttgtcgtcagctcgtgtcgtgagatgttgggttaagtc |
| | | ccgcaacgagcgcaacccttgtgattagttgctacgcaagagcactctaatcagactgccgttgacaaaacggaggaaggtg |
| | | gggatgacgtcaaatcatcatgccctttatgacctgggctacacacgtactacaatggccgtaaacagagagaggcgacaccg |
| | | cgaggtgaagcgaaaccctaaaaacggtctcagttcggattgtaggctgcaacccgcctacatgaagttggaattgctagtaat |
| | | cgcggatcagcatgccgcggtgaatacgttcccgggccttgtacacaccgcccgtcacaccatgggagccggtaatacccga |
| | | agtcagtagtctaaccgcaaggggggcgctgccgaaggtaggattggcgactggggtgaagtcgtaacaaggtagccgtat |
| | | cggaaggtgcggctggatcacctccttt |
| Ruminococcus_ | SEQ ID | attaagagtttgatcctggctcaggacgaacgctggcggcacgcttaacacatgcaagtcgaacgagagaagagaagcttgct |
| bicirculans | NO: 26 | tttctgatctagtggcggacgggtgagtaacacgtgagcaatctgcctttcagagggggataccgattggaaacgatcgttaata |
| | | ccgcataacataattgaaccgcatgatttgattatcaaagatttatcgctgaaagatgagctcgcgtctgattagctagttggtaag |
| | | gtaacggcttaccaaggcgacgatcagtagccggactgagaggttgatcggccacattgggactgagacacggcccagact |
| | | cctacgggaggcagcagtggggaatattgcacaatggaggaaactctgatgcagcgatgccgcgtgagggaagaaggtttt |
| | | aggattgtaaacctctgtcttcagggacgaaaaaaaagacggtacctgaggaggaagctccggctaactacgtgccagcagc |
| | | cgcggtaatacgtagggagcgagcgttgtccggaattactgggtgtaaagggagcgtaggcgggatcgcaagtcagatgtg |
| | | aaaactatgggcttaacccataaactgcatttgaaactgtggttcttgagtgaagtagaggtaagcggaattcctagtgtagcggt |
| | | gaaatgcgtagatattaggaggaacatcagtggcgaaggcggcttactgggctttaactgacgctgaggctcgaaagcgtgg |
| | | ggagcaaacaggattagataccctggtagtccacgccgtaaacgatgattactaggtgtggggggactgaccccttccgtgcc |
| | | gcagcaaacgcaataagtaatccacctggggagtacgaccgcaaggttgaaactcaaaggaattgacgggggcccgcacaa |
| | | gcagtggagtatgtggattaattcgaagcaacgcgaagaaccttaccaggtcttgacatcgtatgcatagctcagagatgagtg |
| | | aaatctcttcggagacatatagacaggtggtgcatggttgtcgtcagctcgtgtcgtgagatgttgggttaagtcccgcaacgag |
| | | cgcaacccttactgttagttgctacgcaagagcactctagcaggactgccgttgacaaaacggaggaaggtggggatgacgt |
| | | caaatcatcatgccccttatgacctgggcctcacacgtactacaatggctgtcaacagagggatgcaaagccgcgaggtggag |
| | | cgaacccctaaaagcagtcttagttcggattgtaggctgcaacccgcctacatgaagtcggaattgctagtaatcgcagatcag |
| | | catgctgcggtgaatacgttcccgggccttgtacacaccgcccgtcacgccatgggagtcggtaacacccgaagcctgtagtc |
| | | taaccgcaaggaggacgcagtcgaaggtgggattgatgactggggtgaagtcgtaacaaggtagccgtatcggaaggtgcg |
| | | gctggatcacctccttt |
| Streptococcus_ | SEQ ID | aatgagagtttgatcctggctcaggacgaacgctggcggcgtgcctaatacatgcaagtagaacgcacagtttataccgtagct |
| gordonii | NO: 27 | tgctacaccatagactgtgagttgcgaacgggtgagtaacgcgtaggtaacctgcctggtagcgggggataactattggaaac |
| | | gatagctaataccgcataatattaattattgcatgataattgattgaaagatgcaactgcatcactaccagatggacctgcgttgtat |
| | | tagctagtaggtgaggtaacggctcacctaggcgacgatacatagccgacctgagagggtgatcggccacactgggactga |
| | | gacacggcccagactcctacgggaggcagcagtagggaatcttcggcaatggacgaaagtctgaccgagcaacgccgcgt |
| | | gagtgaagaaggttttcggatcgtaaagctctgttgtaagagaagaacgggtgtgagagtggaaagttcacactgtgacggtat |
| | | cttaccagaaagggacggctaactacgtgccagcagccgcggtaatacgtaggtcccgagcgttgtccggatttattgggcgt |
| | | aaagcgagcgcaggcggttagataagtctgaagttaaaggctgtggcttaaccatagtacgctttggaaactgtttaacttgagt |
| | | gcagaaggggagagtggaattccatgtgtagcggtgaaatgcgtagatatatggaggaacaccggtggcgaaagcggctct |
| | | ctggtctgtaactgacgctgaggctcgaaagcgtggggagcgaacaggattagataccctggtagtccacgccgtaaacgat |
| | | gagtgctaggtgttaggccctttccggggcttagtgccgcagctaacgcattaagcactccgcctggggagtacgaccgcaag |
| | | gttgaaactcaaaggaattgacgggggcccgcacaagcggtggagcatgtggtttaattcgaagcaacgcgaagaaccttac |
| | | caggtcttgacatcccgatgcccgctctagagatagagttttacttcggtacatcggtgacaggtggtgcatggttgtcgtcagct |
| | | cgtgtcgtgagatgttgggttaagtcccgcaacgagcgcaaccatattgttagttgccatcattcagttgggcactctagcgaga |
| | | ctgccggtaataaaccggaggaaggtggggatgacgtcaaatcatcatgccccttatgacctgggctacacacgtgctacaat |
| | | ggctggtacaacgagtcgcaagtcggtgacggcaagctaatctcttaaagccagtctcagttcggattgtaggctgcaactcgc |
| | | ctacatgaagtcggaatcgctagtaatcgcggatcagcacgccgcggtgaatacgttcccgggccttgtacacaccgcccgtc |
| | | acaccacgagagtttgtaacacccgaagtcggtgaggtaaccttttaggagccagccgcctaaggtgggatagatgattgggg |
| | | tgaagtcgtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgcacagtttataccgtagct |
| gordonii | NO: 28 | tgctacaccatagactgtgagttgcgaacgggtgagtaacgcgtaggtaacctgcctggtagcgggggataactattggaaac |
| | | gatagctaataccgcataatattaattattgcatgataattgattgaaagatgcaactgcatcactaccagatggacctgcgttgtat |
| | | tagctagtaggtgaggtaacggctcacctaggcgacgatacatagccgacctgagagggtgatcggccacactgggactga |
| | | gacacggcccagactcctacgggaggcagcagtagggaatcttcggcaatggacgaaagtctgaccgagcaacgccgcgt |
| | | gagtgaagaaggttttcggatcgtaaagctctgttgtaagagaagaacgggtgtgagagtggaaagttcacactgtgacggtat |
| | | cttaccagaaagggacggctaactacgtgccagcagccgcggtaatacgtaggtcccgagcgttgtccggatttattgggcgt |
| | | aaagcgagcgcaggcggttagataagtctgaagttaaaggctgtggcttaaccatagtacgctttggaaactgtttaacttgagt |
| | | gcagaaggggagagtggaattccatgtgtagcggtgaaatgcgtagatatatggaggaacaccggtggcgaaagcggctct |
| | | ctggtctgtaactgacgctgaggctcgaaagcgtggggagcgaacaggattagataccctggtagtccacgccgtaaacgat |
| | | gagtgctaggtgttaggccctttccggggcttagtgccgcagctaacgcattaagcactccgcctggggagtacgaccgcaag |
| | | gttgaaactcaaaggaattgacgggggcccgcacaagcggtggagcatgtggtttaattcgaagcaacgcgaagaaccttac |
| | | caggtcttgacatcccgatgcccgctctagagatagagttttacttcggtacatcggtgacaggtggtgcatggttgtcgtcagct |
| | | cgtgtcgtgagatgttgggttaagtcccgcaacgagcgcaacccttattgttagttgccatcattcagttgggcactctagcgaga |
| | | ctgccggtaataaaccggaggaaggtggggatgacgtcaaatcatcatgccccttatgacctgggctacacacgtgctacaat |
| | | ggctggtacaacgagtcgcaagtcggtgacggcaagctaatctcttaaagccagtctcagttcggattgtaggctgcaactcgc |
| | | ctacatgaagtcggaatcgctagtaatcgcggatcagcacgccgcggtgaatacgttcccgggccttgtacacaccgcccgtc |
| | | acaccacgagagtttgtaacacccgaagtcggtgaggtaaccttttaggagccagccgcctaaggtgggatagatgattgggg |
| | | tgaagtcgtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgcacagtttataccgtagct |
| gordonii | NO: 29 | tgctacaccatagactgtgagttgcgaacgggtgagtaacgcgtaggtaacctgcctggtagcgggggataactattggaaac |
| | | gatagctaataccgcataatattaattattgcatgataattgattgaaagatgcaactgcatcactaccagatggacctgcgttgtat |
| | | tagctagtaggtgaggtaacggctcacctaggcgacgatacatagccgacctgagagggtgatcggccacactgggactga |
| | | gacacggcccagactcctacgggaggcagcagtagggaatcttcggcaatggacgaaagtctgaccgagcaacgccgcgt |
| | | gagtgaagaaggttttcggatcgtaaagctctgttgtaagagaagaacgggtgtgagagtggaaagttcacactgtgacggtat |
| | | cttaccagaaagggacggctaactacgtgccagcagccgcggtaatacgtaggtcccgagcgttgtccggatttattgggcgt |
| | | aaagcgagcgcaggcggttagataagtctgaagttaaaggctgtggcttaaccatagtacgctttggaaactgtttaacttgagt |
| | | gcagaaggggagagtggaattccatgtgtagcggtgaaatgcgtagatatatggaggaacaccggtggcgaaagcggctct |
| | | ctggtctgtaactgacgctgaggctcgaaagcgtggggagcgaacaggattagataccctggtagtccacgccgtaaacgat |
| | | gagtgctaggtgttaggccctttccggggcttagtgccgcagctaacgcattaagcactccgcctggggagtacgaccgcaag |
| | | gttgaaactcaaaggaattgacgggggcccgcacaagcggtggagcatgtggtttaattcgaagcaacgcgaagaaccttac |
| | | caggtcttgacatcccgatgcccgctctagagatagagttttacttcggtacatcggtgacaggtggtgcatggttgtcgtcagct |
| | | cgtgtcgtgagatgttgggttaagtcccgcaacgagcgcaacccttattgttagttgccatcattcagttgggcactctagcgaga |
| | | ctgccggtaataaaccggaggaaggtggggatgacgtcaaatcatcatgccccttatgacctgggctacacacgtgctacaat |
| | | ggctggtacaacgagtcgcaagtcggtgacggcaagctaatctcttaaagccagtctcagttcggattgtaggctgcaactcgc |
| | | ctacatgaagtcggaatcgctagtaatcgcggatcagcacgccgcggtgaatacgttcccgggccttgtacacaccgcccgtc |
| | | acaccacgagagtttgtaacacccgaagtcggtgaggtaaccttttaggagccagccgcctaaggtgggatagatgattgggg |
| | | tgaagtcgtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgcacagtttataccgtagct |
| gordonii | NO: 30 | tgctacaccataaactgtgagttgcgaacgggtgagtaacgcgtaggtaacctgcctggtagcgggggataactattggaaac |
| | | gatagctaataccgcataatattgattattgcatgataattgattgaaagatgctattgcatcactaccagatggacctgcgttgtatt |
| | | agctagtaggtgaggtaacggctcacctaggcgacgatacatagccgacctgagagggtgatcggccacactgggactgag |
| | | acacggcccagactcctacgggaggcagcagtagggaatcttcggcaatggacgaaagtctgaccgagcaacgccgcgtg |
| | | agtgaagaaggttttcggatcgtaaagctctgttgtaagagaagaacgggtgtgagagtggaaagttcacactgtgacggtatc |
| | | ttaccagaaagggacggctaactacgtgccagcagccgcggtaatacgtaggtcccgagcgttgtccggatttattgggcgta |
| | | aagcgagcgcaggcggttagataagtctgaagttaaaggctgtggcttaaccatagtacgctttggaaactgtttaacttgagtg |
| | | cagaaggggagagtggaattccatgtgtagcggtgaaatgcgtagatatatggaggaacaccggtggcgaaagcggctctct |
| | | ggtctgtaactgacgctgaggctcgaaagcgtggggagcgaacaggattagataccctggtagtccacgccgtaaacgatga |
| | | gtgctaggtgttaggccctttccggggcttagtgccgcagctaacgcattaagcactccgcctggggagtacgaccgcaaggt |
| | | tgaaactcaaaggaattgacgggggcccgcacaagcggtggagcatgtggtttaattcgaagcaacgcgaagaaccttacca |
| | | ggtcttgacatcccgatgcccgctctagagatagagttttacttcggtacatcggtgacaggtggtgcatggttgtcgtcagctcg |
| | | tgtcgtgagatgttgggttaagtcccgcaacgagcgcaacccttattgttagttgccatcattcagttgggcactctagcgagact |
| | | gccggtaataaaccggaggaaggtggggatgacgtcaaatcatcatgccccttatgacctgggctacacacgtgctacaatgg |
| | | ctggtacaacgagtcgcaagtcggtgacggcaagctaatctcttaaagccagtctcagttcggattgtaggctgcaactcgcct |
| | | acatgaagtcggaatcgctagtaatcgcggatcagcacgccgcggtgaatacgttcccgggccttgtacacaccgcccgtcac |
| | | accacgagagtttgtaacacccgaagtcggtgaggtaaccttttaggagccagccgcctaaggtgggatagatgattggggtg |
| | | aagtcgtaacaaggtagccgtatcggaaggtgcggctggatcacctcctt |
| Akkermansia_ | SEQ ID | atggagagtttgattctggctcagaacgaacgctggcggcgtggataagacatgcaagtcgaacgagagaattgctagcttgc |
| muciniphila | NO: 31 | taataattctctagtggcgcacgggtgagtaacacgtgagtaacctgcccccgagagcgggatagccctgggaaactgggatt |
| | | aataccgcatagtatcgaaagattaaagcagcaatgcgcttggggatgggctcgcggcctattagttagttggtgaggtaacgg |
| | | ctcaccaaggcgatgacgggtagccggtctgagaggatgtccggccacactggaactgagacacggtccagacacctacg |
| | | ggtggcagcagtcgagaatcattcacaatgggggaaaccctgatggtgcgacgccgcgtgggggaatgaaggtcttcggatt |
| | | gtaaacccctgtcatgtgggagcaaattaaaaagatagtaccacaagaggaagagacggctaactctgtgccagcagccgcg |
| | | gtaatacagaggtctcaagcgttgttcggaatcactgggcgtaaagcgtgcgtaggctgtttcgtaagtcgtgtgtgaaaggcg |
| | | cgggctcaacccgcggacggcacatgatactgcgagactagagtaatggagggggaaccggaattctcggtgtagcagtga |
| | | aatgcgtagatatcgagaggaacactcgtggcgaaggcgggttcctggacattaactgacgctgaggcacgaaggccaggg |
| | | gagcgaaagggattagatacccctgtagtcctggcagtaaacggtgcacgcttggtgtgcggggaatcgaccccctgcgtgc |
| | | cggagctaacgcgttaagcgtgccgcctggggagtacggtcgcaagattaaaactcaaagaaattgacggggacccgcaca |
| | | agcggtggagtatgtggcttaattcgatgcaacgcgaagaaccttacctgggcttgacatgtaatgaacaacatgtgaaagcat |
| | | gcgactcttcggaggcgttacacaggtgctgcatggccgtcgtcagctcgtgtcgtgagatgtttggttaagtccagcaacgag |
| | | cgcaacccctgttgccagttaccagcacgtgaaggtggggactctggcgagactgcccagatcaactgggaggaaggtggg |
| | | gacgacgtcaggtcagtatggcccttatgcccagggctgcacacgtactacaatgcccagtacagagggggccgaagccgc |
| | | gaggcggaggaaatcctaaaaactgggcccagttcggactgtaggctgcaacccgcctacacgaagccggaatcgctagta |
| | | atggcgcatcagctacggcgccgtgaatacgttcccgggtcttgtacacaccgcccgtcacatcatggaagccggtcgcaccc |
| | | gaagtatctgaagccaaccgcaaggaggcagggtcctaaggtgagactggtaactgggatgaagtcgtaacaaggtagccg |
| | | taggggaacctgcggctggatcacctccttt |
| Akkermansia_ | SEQ ID | atggagagtttgattctggctcagaacgaacgctggcggcgtggataagacatgcaagtcgaacgagagaattgctagcttgc |
| muciniphila | NO: 32 | taataattctctagtggcgcacgggtgagtaacacgtgagtaacctgcccccgagagcgggatagccctgggaaactgggatt |
| | | aataccgcatagtatcgaaagattaaagcagcaatgcgcttggggatgggctcgcggcctattagttagttggtgaggtaacgg |
| | | ctcaccaaggcgatgacgggtagccggtctgagaggatgtccggccacactggaactgagacacggtccagacacctacg |
| | | ggtggcagcagtcgagaatcattcacaatgggggaaaccctgatggtgcgacgccgcgtgggggaatgaaggtcttcggatt |
| | | gtaaacccctgtcatgtgggagcaaattaaaaagatagtaccacaagaggaagagacggctaactctgtgccagcagccgcg |
| | | gtaatacagaggtctcaagcgttgttcggaatcactgggcgtaaagcgtgcgtaggctgtttcgtaagtcgtgtgtgaaaggcg |
| | | cgggctcaacccgcggacggcacatgatactgcgagactagagtaatggagggggaaccggaattctcggtgtagcagtga |
| | | aatgcgtagatatcgagaggaacactcgtggcgaaggcgggttcctggacattaactgacgctgaggcacgaaggccaggg |
| | | gagcgaaagggattagatacccctgtagtcctggcagtaaacggtgcacgcttggtgtgcggggaatcgaccccctgcgtgc |
| | | cggagctaacgcgttaagcgtgccgcctggggagtacggtcgcaagattaaaactcaaagaaattgacggggacccgcaca |
| | | agcggtggagtatgtggcttaattcgatgcaacgcgaagaaccttacctgggcttgacatgtaatgaacaacatgtgaaagcat |
| | | gcgactcttcggaggcgttacacaggtgctgcatggccgtcgtcagctcgtgtcgtgagatgtttggttaagtccagcaacgag |
| | | cgcaacccctgttgccagttaccagcacgtgaaggtggggactctggcgagactgcccagatcaactgggaggaaggtggg |
| | | gacgacgtcaggtcagtatggcccttatgcccagggctgcacacgtactacaatgcccagtacagagggggccgaagccgc |
| | | gaggcggaggaaatcctaaaaactgggcccagttcggactgtaggctgcaacccgcctacacgaagccggaatcgctagta |
| | | atggcgcatcagctacggcgccgtgaatacgttcccgggtcttgtacacaccgcccgtcacatcatggaagccggtcgcaccc |
| | | gaagtatctgaagccaaccgcaaggaggcagggtcctaaggtgagactggtaactgggatgaagtcgtaacaaggtagccg |
| | | taggggaacctgcggctggatcacctccttt |
| Akkermansia_ | SEQ ID | atggagagtttgattctggctcagaacgaacgctggcggcgtggataagacatgcaagtcgaacgagagaattgctagcttgc |
| muciniphila | NO: 33 | taataattctctagtggcgcacgggtgagtaacacgtgagtaacctgcccccgagagcgggatagccctgggaaactgggatt |
| | | aataccgcatagtatcgaaagattaaagcagcaatgcgcttggggatgggctcgcggcctattagttagttggtgaggtaacgg |
| | | ctcaccaaggcgatgacgggtagccggtctgagaggatgtccggccacactggaactgagacacggtccagacacctacg |
| | | ggtggcagcagtcgagaatcattcacaatgggggaaaccctgatggtgcgacgccgcgtgggggaatgaaggtcttcggatt |
| | | gtaaacccctgtcatgtgggagcaaattaaaaagatagtaccacaagaggaagagacggctaactctgtgccagcagccgcg |
| | | gtaatacagaggtctcaagcgttgttcggaatcactgggcgtaaagcgtgcgtaggctgtttcgtaagtcgtgtgtgaaaggcg |
| | | cgggctcaacccgcggacggcacatgatactgcgagactagagtaatggagggggaaccggaattctcggtgtagcagtga |
| | | aatgcgtagatatcgagaggaacactcgtggcgaaggcgggttcctggacattaactgacgctgaggcacgaaggccaggg |
| | | gagcgaaagggattagatacccctgtagtcctggcagtaaacggtgcacgcttggtgtgcggggaatcgaccccctgcgtgc |
| | | cggagctaacgcgttaagcgtgccgcctggggagtacggtcgcaagattaaaactcaaagaaattgacggggacccgcaca |
| | | agcggtggagtatgtggcttaattcgatgcaacgcgaagaaccttacctgggcttgacatgtaatgaacaacatgtgaaagcat |
| | | gcgactcttcggaggcgttacacaggtgctgcatggccgtcgtcagctcgtgtcgtgagatgtttggttaagtccagcaacgag |
| | | cgcaacccctgttgccagttaccagcacgtgaaggtggggactctggcgagactgcccagatcaactgggaggaaggtggg |
| | | gacgacgtcaggtcagtatggcccttatgcccagggctgcacacgtactacaatgcccagtacagagggggccgaagccgc |
| | | gaggcggaggaaatcctaaaaactgggcccagttcggactgtaggctgcaacccgcctacacgaagccggaatcgctagta |
| | | atggcgcatcagctacggcgccgtgaatacgttcccgggtcttgtacacaccgcccgtcacatcatggaagccggtcgcaccc |
| | | gaagtatctgaagccaaccgcaaggaggcagggtcctaaggtgagactggtaactgggatgaagtcgtaacaaggtagccg |
| | | taggggaacctgcggctggatcacctccttt |
| Eubacterium_ | SEQ ID | atgagagtttgatcctggctcaggatgaacgctggcggcgtgcttaacacatgcaagtcgaacgaagcactttatttgatttcctt |
| _rectale | NO: 34 | cgggactgattattttgtgactgagtggcggacgggtgagtaacgcgtgggtaacctgccagtacagggggataacagagg |
| | | aaacggctgctaataccgcataagcgcacggcatcgcatgatgcagtgtgaaaaactccggtggtataagatggacccgcgtt |
| | | ggattagctagttggtgaggtaacggcccaccaaggcgacgatccatagccgacctgagagggtgaccggccacattggga |
| | | ctgagacacggcccaaactcctacgggaggcagcagtggggaatattgcacaatgggcgaaagcctgatgcagcgacgcc |
| | | gcgtgagcgaagaagtatttcggtatgtaaagctctatcagcagggaagataatgacggtacctgactaagaagcaccggcta |
| | | aatacgtgccagcagccgcggtaatacgtatggtgcaagcgttatccggatttactgggtgtaaagggagcgcaggcggtgc |
| | | ggcaagtctgatgtgaaagcccggggctcaaccccggtactgcattggaaactgtcgtactagagtgtcggaggggtaagcg |
| | | gaattcctagtgtagcggtgaaatgcgtagatattaggaggaacaccagtggcgaaggcggcttactggacgataactgacgc |
| | | tgaggctcgaaagcgtggggagcaaacaggattagataccctggtagtccacgccgtaaacgatgaatactaggtgttggga |
| | | agcattgcttctcggtgccgtcgcaaacgcagtaagtattccacctggggagtacgttcgcaagaatgaaactcaaaggaattg |
| | | acggggacccgcacaagcggtggagcatgtggtttaattcgaagcaacgcgaagaaccttaccaagtcttgacatccttctga |
| | | ccggtacttaaccgtaccttctcttcggagcaggagtgacaggtggtgcatggttgtcgtcagctcgtgtcgtgagatgttgggtt |
| | | aagtcccgcaacgagcgcaacccttatctttagtagccagcggttcggccgggcactctagagagactgccagggataacct |
| | | ggaggaaggcggggatgacgtcaaatcatcatgccccttatgacttgggctacacacgtgctacaatggcgtaaacaaaggg |
| | | aagcaaagctgtgaagccgagcaaatctcaaaaataacgtctcagttcggactgtagtctgcaacccgactacacgaagctgg |
| | | aatcgctagtaatcgcagatcagaatgctgcggtgaatacgttcccgggtcttgtacacaccgcccgtcacaccatgggagttg |
| | | ggaatgcccgaagccagtgacctaaccgaaaggaaggagctgtcgaaggcaggctcgataactggggtgaagtcgtaaca |
| | | aggtagccgtatcggaaggtgcggctggatcacctccttt |
| Eubacterium_ | SEQ ID | atgagagtttgatcctggctcaggatgaacgctggcggcgtgcttaacacatgcaagtcgaacgaagcactttatttgatttcctt |
| rectale | NO: 35 | cgggactgattattttgtgactgagtggcggacgggtgagtaacgcgtgggtaacctgccttgtacagggggataacagttgg |
| | | aaacggctgctaataccgcataagcgcacagcatcgcatgatgcagtgtgaaaaactccggtggtataagatggacccgcgtt |
| | | ggattagctagttggtgaggtaacggcccaccaaggcgacgatccatagccgacctgagagggtgaccggccacattggga |
| | | ctgagacacggcccaaactcctacgggaggcagcagtggggaatattgcacaatgggcgaaagcctgatgcagcgacgcc |
| | | gcgtgagcgaagaagtatttcggtatgtaaagctctatcagcagggaagataatgacggtacctgactaagaagcaccggcta |
| | | aatacgtgccagcagccgcggtaatacgtatggtgcaagcgttatccggatttactgggtgtaaagggagcgcaggcggtgc |
| | | ggcaagtctgatgtgaaagcccggggctcaaccccggtactgcattggaaactgtcgtactagagtgtcggaggggtaagcg |
| | | gaattcctagtgtagcggtgaaatgcgtagatattaggaggaacaccagtggcgaaggcggcttactggacgataactgacgc |
| | | tgaggctcgaaagcgtggggagcaaacaggattagataccctggtagtccacgccgtaaacgatgaatactaggtgttggga |
| | | agcattgatctcggtgccgtcgcaaacgcagtaagtattccacctggggagtacgttcgcaagaatgaaactcaaaggaattg |
| | | acggggacccgcacaagcggtggagcatgtggtttaattcgaagcaacgcgaagaaccttaccaagtcttgacatccttctga |
| | | ccggtacttaaccgtaccttctcttcggagcaggagtgacaggtggtgcatggttgtcgtcagctcgtgtcgtgagatgttgggtt |
| | | aagtcccgcaacgagcgcaacccttatctttagtagccagcggtccggccgggcactctagagagactgccagggataacct |
| | | ggaggaaggcggggatgacgtcaaatcatcatgccccttatgacttgggctacacacgtgctacaatggcgtaaacaaaggg |
| | | aagcaaagctgtgaagccgagcaaatctcaaaaataacgtctcagttcggactgtagtctgcaacccgactacacgaagagg |
| | | aatcgctagtaatcgcagatcagaatgctgcggtgaatacgttcccgggtcttgtacacaccgcccgtcacaccatgggagttg |
| | | ggaatgcccgaagccagtgacctaaccgaaaggaaggagctgtcgaaggcaggctcgataactggggtgaagtcgtaaca |
| | | aggtagccgtatcggaaggtgcggctggatcacctccttt |
| Eubacterium_ | SEQ ID | atgagagtttgatcctggctcaggatgaacgctggcggcgtgcttaacacatgcaagtcgaacgaagcactttatttgatttcctt |
| rectale | NO: 36 | cgggactgattattttgtgactgagtggcggacgggtgagtaacgcgtgggtaacctgccttgtacagggggataacagttgg |
| | | aaacggctgctaataccgcataagcgcacggcatcgcatgatgcagtgtgaaaaactccggtggtataagatggacccgcgtt |
| | | ggattagctagttggtgaggtaacggcccaccaaggcgacgatccatagccgacctgagagggtgaccggccacattggga |
| | | ctgagacacggcccaaactcctacgggaggcagcagtggggaatattgcacaatgggcgaaagcctgatgcagcgacgcc |
| | | gcgtgagcgaagaagtatttcggtatgtaaagctctatcagcagggaagataatgacggtacctgactaagaagcaccggcta |
| | | aatacgtgccagcagccgcggtaatacgtatggtgcaagcgttatccggatttactgggtgtaaagggagcgcaggcggtgc |
| | | ggcaagtctgatgtgaaagcccggggctcaaccccggtactgcattggaaactgtcgtactagagtgtcggaggggtaagcg |
| | | gaattcctagtgtagcggtgaaatgcgtagatattaggaggaacaccagtggcgaaggcggcttactggacgataactgacgc |
| | | tgaggctcgaaagcgtggggagcaaacaggattagataccctggtagtccgcgccgtaaacgatgaatactaggtgttggga |
| | | agcattgcttctcggtgccgtcgcaaacgcagtaagtattccacctggggagtacgttcgcaagaatgaaactcaaaggaattg |
| | | acggggacccgcacaagcggtggagcatgtggtttaattcgaagcaacgcgaagaaccttaccaagtcttgacatccttctga |
| | | ccggtacttaaccgtaccttctcttcggagcaggagtgacaggtggtgcatggttgtcgtcagctcgtgtcgtgagatgttgggtt |
| | | aagtcccgcaacgagcgcaacccttatctttagtagccagcggtttggccgggcactctagagagactgccagggataacctg |
| | | gaggaaggcggggatgacgtcaaatcatcatgccccttatgacttgggctacacacgtgctacaatggcgtaaacaaaggga |
| | | agcaaagctgtgaagccgagcaaatctcaaaaataacgtctcagttcggactgtagtctgcaacccgactacacgaagctgga |
| | | atcgctagtaatcgcagatcagaatgctgcggtgaatacgttcccgggtcttgtacacaccgcccgtcacaccatgggagttgg |
| | | gaatgcccgaagccagtgacctaaccgaaaggaaggagctgtcgaaggcaggctcgataactggggtgaagtcgtaacaa |
| | | ggtagccgtatcggaaggtgcggctggatcacctccttt |
| Eubacterium_ | SEQ ID | atgagagtttgatcctggctcaggatgaacgctggcggcgtgcttaacacatgcaagtcgaacgaagcactttatttgatttcctt |
| rectale | NO: 37 | cgggactgattattttgtgactgagtggcggacgggtgagtaacgcgtgggtaacctgccttgtacagggggataacagttgg |
| | | aaacggctgctaataccgcataagcgcacggcatcgcatgatgcagtgtgaaaaactccggtggtataagatggacccgcgtt |
| | | ggattagctagttggtgaggtaacggcccaccaaggcgacgatccatagccgacctgagagggtgaccggccacattggga |
| | | ctgagacacggcccaaactcctacgggaggcagcagtggggaatattgcacaatgggcgaaagcctgatgcagcgacgcc |
| | | gcgtgagcgaagaagtatttcggtatgtaaagctctatcagcagggaagataatgacggtacctgactaagaagcaccggcta |
| | | aatacgtgccagcagccgcggtaatacgtatggtgcaagcgttatccggatttactgggtgtaaagggagcgcaggcggtgc |
| | | ggcaagtctgatgtgaaagcccggggctcaaccccggtactgcattggaaactgtcgtactagagtgtcggaggggtaagcg |
| | | gaattcctagtgtagcggtgaaatgcgtagatattaggaggaacaccagtggcgaaggcggcttactggacgataactgacgc |
| | | tgaggctcgaaagcgtggggagcaaacaggattagataccctggtagtccacgccgtaaacgatgaatactaggtgttggga |
| | | agcattgcttctcggtgccgtcgcaaacgcagtaagtattccacctggggagtacgttcgcaagaatgaaactcaaaggaattg |
| | | acggggacccgcacaagcggtggagcatgtggtttaattcgaagcaacgcgaagaaccttaccaagtcttgacatccttctga |
| | | ccggtacttaaccgtaccttctcttcggagcaggagtgacaggtggtgcatggttgtcgtcagctcgtgtcgtgagatgttgggtt |
| | | aagtcccgcaacgagcgcaacccttatctttagtagccagcggttcggccgggcactctagagagactgccagggataacct |
| | | ggaggaaggcggggatgacgtcaaatcatcatgccccttatgacttgggctacacacgtgctacaatggcgtaaacaaaggg |
| | | aagcaaagctgtgaagccgagcaaatctcaaaaataacgtctcagttcggactgtagtctgcaacccgactacacgaagctgg |
| | | aatcgctagtaatcgcagatcagaatgctgcggtgaatacgttcccgggtcttgtacacaccgcccgtcacaccatgggagttg |
| | | ggaatgcccgaagccagtgacctaaccgaaaggaaggagctgtcgaaggcaggctcgataactggggtgaagtcgtaaca |
| | | aggtagccgtatcggaaggtgcggctggatcacctccttt |
| Eubacterium_ | SEQ ID | atgagagtttgatcctggctcaggatgaacgctggcggcgtgcttaacacatgcaagtcgaacgaagcactttatttgatttcctt |
| rectale | NO: 38 | cgggactgattattttgtgactgagtggcggacgggtgagtaacgcgtgggtaacctgccttgtacagggggataacagttgg |
| | | aaacggctgctaataccgcataagcgcacagcatcgcatgatgcagtgtgaaaaactccggtggtataagatggacccgcgtt |
| | | ggattagctagttggtgaggtaacggcccaccaaggcgacgatccatagccgacctgagagggtgaccggccacattggga |
| | | ctgagacacggcccaaactcctacgggaggcagcagtggggaatattgcacaatgggcgaaagcctgatgcagcgacgcc |
| | | gcgtgagcgaagaagtatttcggtatgtaaagctctatcagcagggaagataatgacggtacctgactaagaagcaccggcta |
| | | aatacgtgccagcagccgcggtaatacgtatggtgcaagcgttatccggatttactgggtgtaaagggagcgcaggcggtgc |
| | | ggcaagtctgatgtgaaagcccggggctcaaccccggtactgcattggaaactgtcgtactagagtgtcggaggggtaagcg |
| | | gaattcctagtgtagcggtgaaatgcgtagatattaggaggaacaccagtggcgaaggcggcttactggacgataactgacgc |
| | | tgaggctcgaaagcgtggggagcaaacaggattagataccctggtagtccacgctgtaaacgatgaatactaggtgttgggaa |
| | | gcattgcttctcggtgccgtcgcaaacgcagtaagtattccacctggggagtacgttcgcaagaatgaaactcaaaggaattga |
| | | cggggacccgcacaagcggtggagcatgtggtttaattcgaagcaacgcgaagaaccttaccaagtcttgacatccttctgac |
| | | cggtacttaaccgtaccttctcttcggagcaggagtgacaggtggtgcatggttgtcgtcagctcgtgtcgtgagatgttgggtta |
| | | agtcccgcaacgagcgcaacccttatctttagtagccagcggtctggccgggcactctagagagactgccagggataacctg |
| | | gaggaaggcggggatgacgtcaaatcatcatgccccttatgacttgggctacacacgtgctacaatggcgtaaacaaaggga |
| | | agcgaagctgtgaagccgagcaaatctcaaaaataacgtctcagttcggactgtagtctgcaacccgactacacgaagctgga |
| | | atcgctagtaatcgcagatcagaatgctgcggtgaatacgttcccgggtcttgtacacaccgcccgtcacaccatgggagttgg |
| | | gaatgcccgaagccagtgacctaaccgaaaggaaggagctgtcgaaggcaggctcgataactggggtgaagtcgtaacaa |
| | | ggtagccgtatcggaaggtgcggctggatcacctccttt |
| Eggerthella_ | SEQ ID | acggagagtttgatcctggctcaggatgaacgctggcggcgtgcctaacacatgcaagtcgaacgatgaaaccgccctcggg |
| lenta | NO: 39 | cggacatgaagtggcgaacgggtgagtaacacgtgaccaacctgccccttgctccgggacaaccttgggaaaccgaggcta |
| | | ataccggatactcctcgcccccctcctggggggcccgggaaagcccagacggcaagggatggggtcgcggcccattaggt |
| | | agtaggcggggtaacggcccacctagcccgcgatgggtagccgggttgagagaccgaccggccacattgggactgagata |
| | | cggcccagactcctacgggaggcagcagtggggaattttgcgcaatgggggaaaccctgacgcagcaacgccgcgtgcgg |
| | | gacgacggccttcgggttgtaaaccgctttcagcagggaagaaattcgacggtacctgcagaagaagctccggctaactacgt |
| | | gccagcagccgcggtaatacgtagggagcgagcgttatccggattcattgggcgtaaagagcgcgtaggcggcctctcaag |
| | | cgggatctctaatccgagggctcaacccccggccggatcccgaactgggaggctcgagttcggtagaggcaggcggaattc |
| | | ccggtgtagcggtggaatgcgcagatatcgggaagaacaccgatggcgaaggcagcctgctgggccgcaactgacgctga |
| | | ggcgcgaaagctaggggagcgaacaggattagataccctggtagtcctagccgtaaacgatggatactaggtgtggggggc |
| | | tccgccctccgtgccgcagccaacgcattaagtatcccgcctggggagtacggccgcaaggctaaaactcaaaggaattgac |
| | | gggggcccgcacaagcagcggagcatgtggcttaattcgaagcaacgcgaagaaccttaccagggcttgacatggacgtga |
| | | agccggggaaacccggtggccgagaggagcgtccgcaggtggtgcatggctgtcgtcagctcgtgtcgtgagatgttgggtt |
| | | aagtcccgcaacgagcgcaacccctgccccatgttgccagcattaggttggggactcatgggggactgccggcgtcaagcc |
| | | ggaggaaggtggggacgacgtcaagtcatcatgccctttatgccctgggctgcacacgtgctacaatggccggtacaacggg |
| | | ctgcgagaccgcgaggtcgagcgaatccctcaaagccggccccagttcggatcggaggctgcaacccgcctccgtgaagtc |
| | | ggagttgctagtaatcgcggatcagcatgccgcggtgaatacgttcccgggccttgtacacaccgcccgtcacaccacccga |
| | | gtcgtctgcacccgaagccgccggccgaacccgcaaggggcggaggcgtcgaaggtgtggagggtaaggggggtgaag |
| | | tcgtaacaaggtagccgtaccggaaggtgcggctggatcacctccttt |
| Eggerthella_ | SEQ ID | acggagagtttgatcctggctcaggatgaacgctggcggcgtgcctaacacatgcaagtcgaacgatgaaaccgccctcggg |
| lenta | NO: 40 | cggacatgaagtggcgaacgggtgagtaacacgtgaccaacctgccccttgctccgggacaaccttgggaaaccgaggcta |
| | | ataccggatactcctcgcccccctcctggggggcccgggaaagcccagacggcaagggatggggtcgcggcccattaggt |
| | | agtaggcggggtaacggcccacctagcccgcgatgggtagccgggttgagagaccgaccggccacattgggactgagata |
| | | cggcccagactcctacgggaggcagcagtggggaattttgcgcaatgggggaaaccctgacgcagcaacgccgcgtgcgg |
| | | gacgacggccttcgggttgtaaaccgctttcagcagggaagaaattcgacggtacctgcagaagaagctccggctaactacgt |
| | | gccagcagccgcggtaatacgtagggagcgagcgttatccggattcattgggcgtaaagagcgcgtaggcggcctctcaag |
| | | cgggatctctaatccgagggctcaacccccggccggatcccgaactgggaggctcgagttcggtagaggcaggcggaattc |
| | | ccggtgtagcggtggaatgcgcagatatcgggaagaacaccgatggcgaaggcagcctgctgggccgcaactgacgctga |
| | | ggcgcgaaagctaggggagcgaacaggattagataccctggtagtcctagccgtaaacgatggatactaggtgtggggggc |
| | | tccgccctccgtgccgcagccaacgcattaagtatcccgcctggggagtacggccgcaaggctaaaactcaaaggaattgac |
| | | gggggcccgcacaagcagcggagcatgtggcttaattcgaagcaacgcgaagaaccttaccagggcttgacatggacgtga |
| | | agccggggaaacccggtggccgagaggagcgtccgcaggtggtgcatggctgtcgtcagctcgtgtcgtgagatgttgggtt |
| | | aagtcccgcaacgagcgcaacccctgccccatgttgccagcattaggttggggactcatgggggactgccggcgtcaagcc |
| | | ggaggaaggtggggacgacgtcaagtcatcatgccctttatgccctgggctgcacacgtgctacaatggccggtacaacggg |
| | | ctgcgagaccgcgaggtcgagcgaatccctcaaagccggccccagttcggatcggaggctgcaacccgcctccgtgaagtc |
| | | ggagttgctagtaatcgcggatcagcatgccgcggtgaatacgttcccgggccttgtacacaccgcccgtcacaccacccga |
| | | gtcgtctgcacccgaagccgccggccgaacccgcaaggggcggaggcgtcgaaggtgtggagggtaaggggggtgaag |
| | | tcgtaacaaggtagccgtaccggaaggtgcggctggatcacctccttt |
| Eggerthella_ | SEQ ID | acggagagtttgatcctggctcaggatgaacgctggcggcgtgcctaacacatgcaagtcgaacgatgaaaccgccctcggg |
| lenta | NO: 41 | cggacatgaagtggcgaacgggtgagtaacacgtgaccaacctgccccttgctccgggacaaccttgggaaaccgaggcta |
| | | ataccggatactcctcgcccccctcctggggggcccgggaaagcccagacggcaagggatggggtcgcggcccattaggt |
| | | agtaggcggggtaacggcccacctagcccgcgatgggtagccgggttgagagaccgaccggccacattgggactgagata |
| | | cggcccagactcctacgggaggcagcagtggggaattttgcgcaatgggggaaaccctgacgcagcaacgccgcgtgcgg |
| | | gacgacggccttcgggttgtaaaccgctttcagcagggaagaaattcgacggtacctgcagaagaagaccggctaactacgt |
| | | gccagcagccgcggtaatacgtagggagcgagcgttatccggattcattgggcgtaaagagcgcgtaggcggcctctcaag |
| | | cgggatctctaatccgagggctcaacccccggccggatcccgaactgggaggctcgagttcggtagaggcaggcggaattc |
| | | ccggtgtagcggtggaatgcgcagatatcgggaagaacaccgatggcgaaggcagcctgctgggccgcaactgacgctga |
| | | ggcgcgaaagctaggggagcgaacaggattagataccctggtagtcctagccgtaaacgatggatactaggtgtggggggc |
| | | tccgccctccgtgccgcagccaacgcattaagtatcccgcctggggagtacggccgcaaggctaaaactcaaaggaattgac |
| | | gggggcccgcacaagcagcggagcatgtggcttaattcgaagcaacgcgaagaaccttaccagggcttgacatggacgtga |
| | | agccggggaaacccggtggccgagaggagcgtccgcaggtggtgcatggctgtcgtcagctcgtgtcgtgagatgttgggtt |
| | | aagtcccgcaacgagcgcaacccctgccccatgttgccagcattaggttggggactcatgggggactgccggcgtcaagcc |
| | | ggaggaaggtggggacgacgtcaagtcatcatgccctttatgccctgggctgcacacgtgctacaatggccggtacaacggg |
| | | ctgcgagaccgcgaggtcgagcgaatccctcaaagccggccccagttcggatcggaggctgcaacccgcctccgtgaagtc |
| | | ggagttgctagtaatcgcggatcagcatgccgcggtgaatacgttcccgggccttgtacacaccgcccgtcacaccacccga |
| | | gtcgtctgcacccgaagccgccggccgaacccgcaaggggcggaggcgtcgaaggtgtggagggtaaggggggtgaag |
| | | tcgtaacaaggtagccgtaccggaaggtgcggctggatcacctccttt |
| Burkholderiales_ | SEQ ID | atagagtttgatcctggctcagattgaacgctggcggaacgctttacacatgcaagtcgaacggtaacgcgggagaagcttgct |
| bacterium_ | NO: 42 | tctcccggcgacgagtggcgaacgggtgagtaatacatcggaacgtgtccgctcgtgggggacaaccagccgaaaggttgg |
| 1_1_47 | | ctaataccgcatgagttctacggaagaaagagggggacccgcaagggcctctcgcgagcggagcggccgatgactgattag |
| | | ccggttggtgaggtaacggctcaccaaagcaacgatcagtagctggtctgagaggacgaccagccacactgggactgagac |
| | | acggcccagactcctacgggaggcagcagtggggaattttggacaatgggcgcaagcctgatccagctattccgcgtgtggg |
| | | atgaaggccctcgggttgtaaaccacttttgtagagaacgaaaagacaccttcgaataaagggtgttgctgacggtactctaag |
| | | aataagcaccggctaactacgtgccagcagccgcggtaatacgtagggtgcgagcgttaatcggaattactgggcgtaaagg |
| | | gtgcgcaggcggttgagtaagacagatgtgaaatccccgagcttaactcgggaatggcatatgtgactgctcgactagagtgt |
| | | gtcagagggaggtggaattccacgtgtagcagtgaaatgcgtagatatgtggaagaacaccgatggcgaaggcagcctcctg |
| | | ggacataactgacgctcaggcacgaaagcgtggggagcaaacaggattagataccctggtagtccacgccctaaacgatgtt |
| | | aactagttgttgggaagtaaaattctcagtaacgcagccaacgcgagaagttaaccgcctgggaagtacggtcgcaagactaa |
| | | aactcaaaggaattgacggggacccgcacaagcggtggatgatgtggattaattcgatgcaacgcgaaaaaccttacctaccc |
| | | ttgacatgtcaggaagacttgtaatgagagtgtgcccgcaagggagcctgaacacaggtgctgcatggctgtcgtcagacgt |
| | | gtcgtgagatgttgggttaagtcccgcaacgagcgcaacccttgtcactagttgctacgaaagggcactctagtgagactgccg |
| | | gtgacaaaccggaggaaggtggggatgacgtcaagtcctcatggcccttatgggtagggcttcacacgtcatacaatggtcg |
| | | gaacagagggcagcgaagtcgtgagacggagccaatcccagaaaaccgatcgtagtccggattgcagtctgcaactcgact |
| | | gcatgaagtcggaatcgctagtaatcgcggatcagcatgccgcggtgaatacgttcccgggtcttgtacacaccgcccgtcaa |
| | | acaatgggagtggtgtttaccagaagtcgttagcctaaccgcaaggagggcggcgaccacggtgagcaccgtgactaatgtt |
| | | aagtcgtaacaaggtagccgtaccggaaggtgcggctggatcacctccttt |
| Eubacterium_ | SEQ ID | ttatgagagtttgatcctggctcaggatgaacgctggcggcgtgcttaacacatgcaagtcgaacgaagcatttagaacagatta |
| eligens | NO: 43 | cttcggtttgaagttctttatgactgagtggcggacgggtgagtaacgcgtgggtaacctgccttgtactgggggatagcagctg |
| | | gaaacggctggtaataccgcataagcgcacaatgttgcatgacatggtgtgaaaaactccggtggtataagatggacccgcgt |
| | | ctgattagctagttggtgagataacagcccaccaaggcgacgatcagtagccgacctgagagggtgaccggccacattggga |
| | | ctgagacacggcccagactcctacgggaggcagcagtggggaatattgcacaatggaggaaactctgatgcagcgacgcc |
| | | gcgtgagtgaagaagtaattcgttatgtaaagctctatcagcagggaagatagtgacggtacctgactaagaagctccggctaa |
| | | atacgtgccagcagccgcggtaatacgtatggagcaagcgttatccggatttactgggtgtaaagggagtgtaggtggccatg |
| | | caagtcagaagtgaaaatccggggctcaaccccggaactgcttttgaaactgtaaggctggagtgcaggaggggtgagtgga |
| | | attcctagtgtagcggtgaaatgcgtagatattaggaggaacaccagtggcgaaggcggctcactggactgtaactgacactg |
| | | aggctcgaaagcgtggggagcaaacaggattagataccctggtagtccacgccgtaaacgatgaatactaggtgtcggggc |
| | | ccataagggcttcggtgccgcagcaaacgcaataagtattccacctggggagtacgttcgcaagaatgaaactcaaaggaatt |
| | | gacggggacccgcacaagcggtggagcatgtggtttaattcgaagcaacgcgaagaaccttaccaagtcttgacatcctcttg |
| | | accggtcagtaatgtggcctttccttcgggacaagagagacaggtggtgcatggttgtcgtcagctcgtgtcgtgagatgttgg |
| | | gttaagtcccgcaacgagcgcaacccctatccttagtagccagcagtaagatgggcactctagggagactgccagggataac |
| | | ctggaggaaggtggggatgacgtcaaatcatcatgccccttatgacttgggctacacacgtgctacaatggcgtaaacaaagt |
| | | gaagcgaagtcgtgaggccaagcaaatcacaaaaataacgtctcagttcggattgtagtctgcaactcgactacatgaagctg |
| | | gaatcgctagtaatcgcagatcagaatgctgcggtgaatacgttcccgggtcttgtacacaccgcccgtcacaccatgggagte |
| | | gaaaatgcccgaagtcggtgacctaacgtaagaaggagccgccgaaggcaggtttgataactggggtgaagtcgtaacaag |
| | | gtagccgtatcggaaggtgcggctggatcacctccttt |
| Eubacterium_ | SEQ ID | ttatgagagtttgatcctggctcaggatgaacgctggcggcgtgcttaacacatgcaagtcgaacgaagcatttagaacagatta |
| eligens | NO: 44 | tttcggtatgaagttctttatgactgagtggcggacgggtgagtaacgcgtgggtaacctgccttgtactgggggatagcagctg |
| | | gaaacggctggtaataccgcataagcgcacaatgttgcatgacatggtgtgaaaaactccggtggtataagatggacccgcgt |
| | | ctgattagctagttggtgagataacagcccaccaaggcgacgatcagtagccgacctgagagggtgaccggccacattggga |
| | | ctgagacacggcccagactcctacgggaggcagcagtggggaatattgcacaatggaggaaactctgatgcagcgacgcc |
| | | gcgtgagtgaagaagtaattcgttatgtaaagctctatcagcagggaagatagtgacggtacctgactaagaagctccggctaa |
| | | atacgtgccagcagccgcggtaatacgtatggagcaagcgttatccggatttactgggtgtaaagggagtgtaggtggccatg |
| | | caagtcagaagtgaaaatccggggctcaaccccggaactgcttttgaaactgtaaggctggagtgcaggaggggtgagtgga |
| | | attcctagtgtagcggtgaaatgcgtagatattaggaggaacaccagtggcgaaggcggctcactggactgtaactgacactg |
| | | aggctcgaaagcgtggggagcaaacaggattagataccctggtagtccacgccgtaaacgatgaatactaggtgtcggggc |
| | | ccaaaagggcttcggtgccgcagcaaacgcaataagtattccacctggggagtacgttcgcaagaatgaaactcaaaggaatt |
| | | gacggggacccgcacaagcggtggagcatgtggtttaattcgaagcaacgcgaagaaaccttaccaagtcttgacatcccact |
| | | gaccggacagtaatgtgtcctttccttcgggacagtggtgacaggtggtgcatggttgtcgtcagctcgtgtcgtgagatgttgg |
| | | gttaagtcccgcaacgagcgcaacccctatccttagtagccagcagtaagatgggcactctagggagactgccagggataac |
| | | ctggaggaaggtggggatgacgtcaaatcatcatgccccttatgacttgggctacacacgtgctacaatggcgtaaacaaagt |
| | | gaagcgaagtcgtgaggccaagcaaatcacaaaaataacgtctcagttcggattgtagtctgcaactcgactacatgaagctg |
| | | gaatcgctagtaatcgcagatcagaatgctgcggtgaatacgttcccgggtcttgtacacaccgcccgtcacaccatgggagtc |
| | | gaaaatgcccgaagtcggtgacctaacgtaagaaggagccgccgaaggcaggtttgataactggggtgaagtcgtaacaag |
| | | gtagccgtatcggaaggtgcggctggatcacctccttt |
| Eubacterium_ | SEQ ID | ttatgagagtttgatcctggctcaggatgaacgctggcggcgtgcttaacacatgcaagtcgaacgaagcatttagaacagatta |
| eligens | NO: 45 | tttcggtatgaagttctttatgactgagtggcggacgggtgagtaacgcgtgggtaacctgccttgtactgggggatagcagctg |
| | | gaaacggctggtaataccgcataagcgcacaatgttgcatgacatagtgtgaaaaactccggtggtataagatggacccgcgt |
| | | ctgattagctagttggtgagataacagcccaccaaggcgacgatcagtagccgacctgagagggtgaccggccacattggga |
| | | ctgagacacggcccagactcctacgggaggcagcagtggggaatattgcacaatggaggaaactctgatgcagcgacgcc |
| | | gcgtgagtgaagaagtaattcgttatgtaaagctctatcagcagggaagatagtgacggtacctgactaagaagctccggctaa |
| | | atacgtgccagcagccgcggtaatacgtatggagcaagcgttatccggatttactgggtgtaaagggagtgtaggtggccatg |
| | | caagtcagaagtgaaaatccggggcttaaccccggaactgcttttgaaactgtaaggctggagtgcaggaggggtgagtgga |
| | | attcctagtgtagcggtgaaatgcgtagatattaggaggaacaccagtggcgaaggcggctcactggactgtaactgacactg |
| | | aggctcgaaagcgtggggagcaaacaggattagataccctggtagtccacgccgtaaacgatgaatactaggtgtcggggc |
| | | ccataagggcttcggtgccgcagcaaacgcaataagtattccacctggggagtacgttcgcaagaatgaaactcaaaggaatt |
| | | gacggggacccgcacaagcggtggagcatgtggtttaattcgaagcaacgcgaagaaccttaccaagtcttgacatcccact |
| | | gaccggacagtaatgtgtcctttccttcgggacagtggtgacaggtggtgcatggttgtcgtcagctcgtgtcgtgagatgttgg |
| | | gttaagtcccgcaacgagcgcaacccctatccttagtagccagcagtaagatgggcactctagggagactgccagggataac |
| | | ctggaggaaggtggggatgacgtcaaatcatcatgccccttatgacttgggctacacacgtgctacaatggcgtaaacaaagt |
| | | gaagcgaagtcgtgaggccaagcaaatcacaaaaataacgtctcagttcggattgtagtctgcaactcgactacatgaagctg |
| | | gaatcgctagtaatcgcagatcagaatgctgcggtgaatacgttcccgggtcttgtacacaccgcccgtcacaccatgggagtc |
| | | gaaaatgcccgaagtcggtgacctaacgaaagaaggagccgccgaaggcaggtttgataactggggtgaagtcgtaacaag |
| | | gtagccgtatcggaaggtgcggctggatcacctccttt |
| Eubacterium_ | SEQ ID | ttatgagagtttgatcctggctcaggatgaacgctggcggcgtgcttaacacatgcaagtcgaacgaagcatttagaacagatta |
| eligens | NO: 46 | tttcggtatgaagttctttatgactgagtggcggacgggtgagtaacgcgtgggtaacctgccttgtactgggggatagcagctg |
| | | gaaacggctggtaataccgcataagcgcacaatgttgcatgacatggtgtgaaaaactccggtggtataagatggacccgcgt |
| | | ctgattagctagttggtgagataacagcccaccaaggcgacgatcagtagccgacctgagagggtgaccggccacattggga |
| | | ctgagacacggcccagactcctacgggaggcagcagtggggaatattgcacaatggaggaaactctgatgcagcgacgcc |
| | | gcgtgagtgaagaagtaattcgttatgtaaagctctatcagcagggaagatagtgacggtacctgactaagaagctccggctaa |
| | | atacgtgccagcagccgcggtaatacgtatggagcaagcgttatccggatttactgggtgtaaagggagtgtaggtggccatg |
| | | caagtcagaagtgaaaatccggggctcaaccccggaactgcttttgaaactgtgaggctggagtgcaggaggggtgagtgg |
| | | aattcctagtgtagcggtgaaatgcgtagatattaggaggaacaccagtggcgaaggcggctcactggactgtaactgacact |
| | | gaggctcgaaagcgtggggagcaaacaggattagataccctggtagtccacgccgtaaacgatgaatactaggtgtcgggg |
| | | cccataagggcttcggtgccgcagcaaacgcaataagtattccacctggggagtacgttcgcaagaatgaaactcaaaggaat |
| | | tgacggggacccgcacaagcggtggagcatgtggtttaattcgaagcaacgcgaagaaccttaccaagtcttgacatcccact |
| | | gaccggacagtaatgtgtcctttccttcgggacagtggagacaggtggtgcatggttgtcgtcagctcgtgtcgtgagatgttgg |
| | | gttaagtcccgcaacgagcgcaacccctatccttagtagccagcagtaagatgggcactctagggagactgccagggataac |
| | | ctggaggaaggtggggatgacgtcaaatcatcatgccccttatgacttgggctacacacgtgctacaatggcgtaaacaaagt |
| | | gaagcaaagtcgtgaggccaagcaaatcacaaaaataacgtctcagttcggattgtagtctgcaactcgactacatgaagctgg |
| | | aatcgctagtaatcgcagatcagaatgctgcggtgaatacgttcccgggtcttgtacacaccgcccgtcacaccatgggagtcg |
| | | aaaatgcccgaagtcggtgacctaacgcaagaaggagccgccgaaggcaggtttgataactggggtgaagtcgtaacaagg |
| | | tagccgtatcggaaggtgcggctggatcacctccttt |
| Eubacterium_ | SEQ ID | ttatgagagtttgatcctggctcaggatgaacgctggcggcgtgcttaacacatgcaagtcgaacgaagcatttgcgacagattt |
| eligens | NO: 47 | tttcggaatgaagttgcttatgactgagtggcggacgggtgagtaacgcgtgggtaacctgccttgtactgggggatagcagct |
| | | ggaaacggctggtaataccgcataagcgcacaatgttgcatgacatggtgtgaaaaactccggtggtataagatggacccgc |
| | | gtctgattagctagttggtgagataacagcccaccaaggcgacgatcagtagccgacctgagagggtgaccggccacattgg |
| | | gactgagacacggcccagactcctacgggaggcagcagtggggaatattgcacaatggaggaaactctgatgcagcgacg |
| | | ccgcgtgagtgaagaagtaattcgttatgtaaagctctatcagcagggaagatagtgacggtacctgactaagaagctccggct |
| | | aaatacgtgccagcagccgcggtaatacgtatggagcaagcgttatccggatttactgggtgtaaagggagtgtaggtggcca |
| | | tgcaagtcagaagtgaaaatccggggctcaaccccggaactgcttttgaaactgtaaggctggagtgcaggaggggtgagtg |
| | | gaattcctagtgtagcggtgaaatgcgtagatattaggaggaacaccagtggcgaaggcggctcactggactgtaactgacac |
| | | tgaggctcgaaagcgtggggagcaaacaggattagataccctggtagtccacgccgtaaacgatgaatactaggtgtcgggg |
| | | cccataagggcttcggtgccgcagcaaacgcaataagtattccacctggggagtacgttcgcaagaatgaaactcaaaggaat |
| | | tgacggggacccgcacaagcggtggagcatgtggtttaattcgaagcaacgcgaagaaccttaccaagtcttgacatcccact |
| | | gaccggacagtaatgtgtcctttccttcgggacagtggagacaggtggtgcatggttgtcgtcagctcgtgtcgtgagatgttgg |
| | | gttaagtcccgcaacgagcgcaacccctatccttagtagccagcagtaagatgggcactctagggagactgccagggataac |
| | | ctggaggaaggtggggatgacgtcaaatcatcatgccccttatgacttgggctacacacgtgctacaatggcgtaaacaaagt |
| | | gaagcgaagtcgtgaggccaagcaaatcacaaaaataacgtctcagttcggattgtagtctgcaactcgactacatgaagctg |
| | | gaatcgctagtaatcgcagatcagaatgctgcggtgaatacgttcccgggtcttgtacacaccgcccgtcacaccatgggagtc |
| | | gaaaatgcccgaagtcggtgacctaacgtaagaaggagccgccgaaggcaggtttgataactggggtgaagtcgtaacaag |
| | | gtagccgtatcggaaggtgcggctggatcacctccttt |
| Eubacterium_ | SEQ ID | aacgagagtttgatcctggctcaggatgaacgctggcggcgtgcttaatacatgcaagtcgaacgaagcaccttggacagaat |
| ventriosum | NO: 48 | ccttcgggaggaagaccattgtgactgagtggcggacgggtgagtaacgcgtgggtaacctgccttgtacagggggataaca |
| | | gttggaaacgactgctaataccgcataagcgcacagtaccgcatggtacggtgtgaaaaactccggtggtacaagatggacc |
| | | cgcgtctgattagctggttggtgaggtaacggcccaccaaggcgacgatcagtagccgacttgagagagtgatcggccacatt |
| | | gggactgagacacggcccaaactcctacgggaggcagcagtagggaatattgcacaatgggggaaaccctgatgcagcga |
| | | cgccgcgtgaaggaagaagtatttcggtatgtaaacttctatcagcaaggaagaaaatgacggtacttgactaagaagccccg |
| | | gctaaatacgtgccagcagccgcggtaatacgtatggggcaagcgttatccggatttactgggtgtaaagggagcgtaggcg |
| | | gcatggcaagtcagaagtgaaagcctggggctcaaccccggaattgcttttgaaactgtcaggctagagtgtcggaggggta |
| | | agcggaattcctagtgtagcggtgaaatgcgtagatattaggaggaacaccggtggcgaaggcggcttactggacgattactg |
| | | acgctgaggctcgaaagcgtggggagcaaacaggattagataccctggtagtccacgccgtaaacgatgaatactaggtgtc |
| | | gggggacaatagttcctcggtgccgaagcaaacgcattaagtattccacctggggagtacgttcgcaagaatgaaactcaaag |
| | | gaattgacggggacccgcacaagcggtggagcatgtggtttaattcgaagcaacgcgaagaaccttacctgctcttgacatcc |
| | | cactgacaggtcagtaatgtgaccctttcttcggaacagtggagacaggtggtgcatggttgtcgtcagctcgtgtcgtgagatg |
| | | ttgggttaagtcccgcaacgagcgcaacccttgtctttagtagccagcagtacggctgggcactctagagagactgccaggga |
| | | taacctggaggaaggcggggatgacgtcaaatcatcatgccccttacgagcagggctacacacgtgctacaatggcgtaaac |
| | | aaagggaagcgaccccgtgagggtaagcaaatctcaaaaataacgtctcagttcggattgtagtctgcaactcgactacatgaa |
| | | gctggaatcgctagtaatcgcgaatcagaatgtcgcggtgaatacgttcccgggtcttgtacacaccgcccgtcacaccatgg |
| | | gagttggatatgcccgaagtcagtgacccaaccgtaaggagggagctgccgaaggtggagccgataactggggtgaagtcg |
| | | taacaaggtagccgtatcggaaggtgcggctggatcacctccttt |
| Ruminococcus_ | SEQ ID | tcagagagtttgatcctggctcaggatgaacgctggcggcgtgcttaacacatgcaagtcgaacgggaaacttttcattgaagc |
| obeum | NO: 49 | ttcggcagatttggtctgtttctagtggcggacgggtgagtaacgcgtgggtaacctgccttatacagggggataacaaccaga |
| | | aatggttgctaataccgcataagcgcacaggaccgcatggtccggtgtgaaaaactccggtggtataagatggacccgcgttg |
| | | gattagctagttggcagggtaacggcctaccaaggcgacgatccatagccggcctgagagggtgaacggccacattgggac |
| | | tgagacacggcccagactcctacgggaggcagcagtggggaatattgcacaatgggggaaaccctgatgcagcgacgccg |
| | | cgtgaaggaagaagtatctcggtatgtaaacttctatcagcagggaagatagtgacggtacctgactaagaagccccggctaa |
| | | ctacgtgccagcagccgcggtaatacgtagggggcaagcgttatccggatttactgggtgtaaagggagcgtagacggactg |
| | | gcaagtctgatgtgaaaggcgggggctcaacccctggactgcattggaaactgttagtcttgagtgccggagaggtaagcgg |
| | | aattcctagtgtagcggtgaaatgcgtagatattaggaggaacaccagtggcgaaggcggcttactggacggtaactgacgtt |
| | | gaggctcgaaagcgtggggagcaaacaggattagataccctggtagtccacgccgtaaacgatgaatactaggtgttgggga |
| | | gcaaagctcttcggtgccgccgcaaacgcattaagtattccacctggggagtacgttcgcaagaatgaaactcaaaggaattg |
| | | acggggacccgcacaagcggtggagcatgtggtttaattcgaagcaacgcgaagaaccttaccaagtcttgacatccctctga |
| | | ccgttccttaaccggaactttccttcgggacagaggagacaggtggtgcatggttgtcgtcagctcgtgtcgtgagatgttgggt |
| | | taagtcccgcaacgagcgcaacccctatccccagtagccagcggttcggccgggcactctgaggagactgccagggataac |
| | | ctggaggaaggcggggatgacgtcaaatcatcatgccccttatgatttgggctacacacgtgctacaatggcgtaaacaaagg |
| | | gaagcgagcctgcgaaggtaagcaaatcccaaaaataacgtcccagttcggactgcagtctgcaactcgactgcacgaagct |
| | | ggaatcgctagtaatcgcggatcagaatgccgcggtgaatacgttcccgggtcttgtacacaccgcccgtcacaccatgggag |
| | | tcagtaacgcccgaagtcagtgacctaactgcaaagaaggagctgccgaaggcgggaccgatgactggggtgaagtcgtaa |
| | | caaggtagccgtatcggaaggtgcggctggatcacctccttt |
| Ruminococcus_ | SEQ ID | tcagagagtttgatcctggctcaggatgaacgctggcggcgtgcttaacacatgcaagtcgaacgggaaacttttcattgaagc |
| obeum | NO: 50 | ttcggcagatttggtctgatctagtggcggacgggtgagtaacgcgtgggtaacctgccttatacagggggataacaaccaga |
| | | aatggttgctaataccgcataagcgcacaggaccgcatggtctagtgtgaaaaactccggtggtataagatggacccgcgttg |
| | | gattagctagttggcagggtaacggcctaccaaggcgacgatccatagccggcctgagagggtgaacggccacattgggac |
| | | tgagacacggcccagactcctacgggaggcagcagtggggaatattgcacaatgggggaaaccctgatgcagcgacgccg |
| | | cgtgaaggaagaagtatctcggtatgtaaacttctatcagcagggaagatagtgacggtacctgactaagaagccccggctaa |
| | | ctacgtgccagcagccgcggtaatacgtagggggcaagcgttatccggatttactgggtgtaaagggagcgtagacggactg |
| | | gcaagtctgatgtgaaaggcgggggctcaacccctggactgcattggaaactgttagtcttgagtgccggagaggtaagcgg |
| | | aattcctagtgtagcggtgaaatgcgtagatattaggaggaacaccagtggcgaaggcggcttactggacggtaactgacgtt |
| | | gaggctcgaaagcgtggggagcaaacaggattagataccctggtagtccacgccgtaaacgatgaatactaggtgttgggga |
| | | gcaaagctcttcggtgccgccgcaaacgcattaagtattccacctggggagtacgttcgcaagaatgaaactcaaaggaattg |
| | | acggggacccgcacaagcggtggagcatgtggtttaattcgaagcaacgcgaagaaccttaccaagtcttgacatccctctga |
| | | ccgttccttaaccggaactttccttcgggacaggggagacaggtggtgcatggttgtcgtcagctcgtgtcgtgagatgttgggt |
| | | taagtcccgcaacgagcgcaacccctatccccagtagccagcagtccggctgggcactctgaggagactgccagggataac |
| | | ctggaggaaggcggggatgacgtcaaatcatcatgccccttatgatttgggctacacacgtgctacaatggcgtaaacaaagg |
| | | gaagcaagcctgcgaaggtaagcaaatcccaaaaataacgtcccagttcggactgcagtctgcaactcgactgcacgaagct |
| | | ggaatcgctagtaatcgcggatcagaatgccgcggtgaatacgttcccgggtcttgtacacaccgcccgtcacaccatgggag |
| | | tcagtaacgcccgaagtcagtgacctaactgcaaagaaggagctgccgaaggcgggaccgatgactggggtgaagtcgtaa |
| | | caaggtagccgtatcggaaggtgcggctggatcacctccttt |
| Ruminococcus_ | SEQ ID | gacacggcccagactcctacgggaggcagcagtggggaatattgtacaatgggggaaaccctgatgcagcgacgccgcgt |
| obeum | NO: 51 | gaaggaagaagtatctcggtatgtaaacttctatcagcagggaagatagtgacggtacctgactaagaagccccggctaacta |
| | | cgtgccagcagccgcggtaatacgtagggggcaagcgttatccggatttactgggtgtaaagggagcgtagacggactggc |
| | | aagtctgatgtgaaaggcgggggctcaacccctggactgcattggaaactgttagtcttgagtgccggagaggtaagcggaat |
| | | tcctagtgtagcggtgaaatgcgtagatattaggaggaacaccagtggcgaaggcggcttactggacggtaactgacgttgag |
| | | gctcgaaagcgtggggagcaaacaggattagataccctggtagtccacgccgtaaacgatgaatactaggtgttggggagca |
| | | aagctcttcggtgccgccgcaaacgcattaagtattccacctggggagtacgttcgcaagaatgaaactcaaaggaattgacg |
| | | gggacccgcacaagcggtggagcatgtggtttaattcgaagcaacgcgaagaaccttaccaagtcttgacatccctctgaccg |
| | | actcttaaccgagtctttccttcgggacaggggagacaggtggtgcatggttgtcgtcagctcgtgtcgtgagatgttgggttaa |
| | | gtcccgcaacgagcgcaacccctatccccagtagccagcagtccggctgggcactctgaggagactgccagggataacctg |
| | | gaggaaggcggggatgacgtcaaatcatcatgccccttatgatttgggctacacacgtgctacaatggcgtaaacaaagggaa |
| | | gcaagcctgcgaaggtaagcaaatccccaaaaataacgatcccagttcggactgctgtctgcaactcgactgcacgaagag |
| | | gaattcgctagtaatcgcggatcaaatgccgcggtgaatacgttcccgggatctgtacacacccgcccgtccatccttgggagt |
| | | ctgtacccccgatttagtggacctacc |
| Ruminococcus_ | SEQ ID | tcagagagtttgatcctggctcaggatgaacgctggcggcgtgcttaacacatgcaagtcgaacgggaaacttttcattgaagc |
| obeum | NO: 52 | ttcggcagatttggtctgtttctagtggcggacgggtgagtaacgcgtgggtaacctgccttatacagggggataacaaccaga |
| | | aatggttgctaataccgcataagcgcacaggaccgcatggtccggtgtgaaaaactccggtggtataagatggacccgcgttg |
| | | gattagctagttggcagggtaacggcctaccaaggcgacgatccatagccggcctgagagggtgaacggccacattgggac |
| | | tgagacacggcccagactcctacgggaggcagcagtggggaatattgcacaatgggggaaaccctgatgcagcgacgccg |
| | | cgtgaaggaagaagtatctcggtatgtaaacttctatcagcagggaagatagtgacggtacctgactaagaagccccggctaa |
| | | ctacgtgccagcagccgcggtaatacgtagggggcaagcgttatccggatttactgggtgtaaagggagcgtagacggactg |
| | | gcaagtctgatgtgaaaggcgggggctcaacccctggactgcattggaaactgttagtcttgagtgccggagaggtaagcgg |
| | | aattcctagtgtagcggtgaaatgcgtagatattaggaggaacaccagtggcgaaggcggcttactggacggtaactgacgtt |
| | | gaggctcgaaagcgtggggagcaaacaggattagataccctggtagtccacgccgtaaacgatgaatactaggtgttgggga |
| | | gcaaagctcttcggtgccgccgcaaacgcattaagtattccacctggggagtacgttcgcaagaatgaaactcaaaggatttga |
| | | cgggggacccgcacagcggtggagcatgtggtttattcgagcacggcgaagaaccttaccaagtctgacatcccct |
| Ruminococcus_ | SEQ ID | tcagagagtttgatcctggctcaggatgaacgctggcggcgtgcttaacacatgcaagtcgaacgggaaaccttttattgaagc |
| obeum | NO: 53 | ttcggcagatttagctggtttctagtggcggacgggtgagtaacgcgtgggtaacctgccttatacagggggataacaaccaga |
| | | aatggttgctaataccgcataagcgcacaggaccgcatggtccggtgtgaaaaactccggtggtataagatggacccgcgttg |
| | | gattagctagttggcagggtaacggcctaccaaggcgacgatccatagccggcctgagagggtgaacggccacattgggac |
| | | tgagacacggcccagactcctacgggaggcagcagtggggaatattgcacaatgggggaaaccctgatgcagcgacgccg |
| | | cgtgaaggaagaagtatctcggtatgtaaacttctatcagcagggaagatagtgacggtacctgactaagaagccccggctaa |
| | | ctacgtgccagcagccgcggtaatacgtagggggcaagcgttatccggatttactgggtgtaaagggagcgtagacggactg |
| | | gcaagtctgatgtgaaaggcgggggctcaacccctggactgcattggaaactgttagtcttgagtgccggagaggtaagcgg |
| | | aattcctagtgtagcggtgaaatgcgtagatattaggaggaacaccagtggcgaaagcggcttactggacggtaactgacgtt |
| | | gaggctcgaaagcgtggggagcaaacaggattagatacctggtagtccacgccgtaaacgatgaatactagtgttgggggag |
| | | caaagctcttcgtgccgccgcaaacgcattagtattccacctggggagtacgttcgcaagaatgaaactcaaaggaattgaccg |
| | | gcacccacaaacgcgtgagtcagggtgttaa |
| Ruminococcus_ | SEQ ID | aacgagagtttgatcctggctcaggatgaacgctggcggcgtgcctaacacatgcaagtcgagcgaagcactttgcttagattc |
| torques | NO: 54 | ttcggatgaagaggattgtgactgagcggcggacgggtgagtaacgcgtgggtaacctgcctcatacagggggataacagtt |
| | | agaaatgactgctaataccgcataagaccacagcaccgcatggtgcgggggtaaaaactccggtggtatgagatggacccgc |
| | | gtctgattagctagttggtaaggtaacggcttaccaaggcgacgatcagtagccgacctgagagggtggccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatcccg |
| | | ctgaccggacggtaatgcgtccttcccttcggggcagcggagacaggtggtgcatggttgtcgtcagctcgtgtcgtgagatgt |
| | | tgggttaagtcccgcaacgagcgcaacccctatctttagtagccagcggccaggccgggcactctagagagactgccgggg |
| | | ataacccggaggaaggtggggatgacgtcaaatcatcatgccccttatgagcagggctacacacgtgctacaatggcgtaaa |
| | | caaagggaagcgagaccgcgaggtggagcaaatcccaaaaataacgtctcagttcggattgtagtctgcaactcgactacatg |
| | | aagctggaatcgctagtaatcgcgaatcagaatgtcgcggtgaatacgttcccgggtcttgtacacaccgcccgtcacaccatg |
| | | ggagtcagtaacgcccgaagtcagtgacccaaccgtaaggagggagctgccgaaggcgggaccgataactgggggtgaa |
| | | gtcgtaacaaggtagccgtatcggaaggtgcggctggatcacctccttt |
| Ruminococcus_ | SEQ ID | aacgagagtttgatcctggctcaggatgaacgctggcggcgtgcctaacacatgcaagtcgagcgaagcactttgcttagattc |
| torques | NO: 55 | ttcggatgaagaggattgtgactgagcggcggacgggtgagtaacgcgtgggtaacctgcctcatacagggggataacagtt |
| | | agaaatgactgctaataccgcataagaccacagcaccgcatggtgcgggggtaaaaactccggtggtatgagatggacccgc |
| | | gtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatcccg |
| | | ctgaccggacggtaatgcgtccttcccttcggggcagcggagacaggtggtgcatggttgtcgtcagctcgtgtcgtgagatgt |
| | | tgggttaagtccccgcaacgagcgcaacccctatctttagtagccagcggccaggccgggcactctagagagactgccggg |
| | | gataacccggaggaaggtggggatgacgtcaaatcatcatgccccttatgagcagggctacacacgtgctacaatggcgtaa |
| | | acaaagggaagcgagaccgcgaggtggagcaaatcccaaaaataacgtctcagttcggattgtagtctgcaactcgactacat |
| | | gaagctggaatcgctagtaatcgcgaatcagaatgtcgcggtgaatacgttcccgggtcttgtacacaccgcccgtcacaccat |
| | | gggagtcagtaacgcccgaagtcagtgacccaaccgtaaggagggagctgccgaaggcgggaccgataactggggtgaa |
| | | gtcgtaacaaggtagccgtatcggaaggtgcggctggatcacctccttt |
| Ruminococcus_ | SEQ ID | aacgagagtttgatcctggctcaggatgaacgctggcggcgtgcctaacacatgcaagtcgagcgaagcactttgcttagattc |
| torques | NO: 56 | ttcggatgaagaggattgtgactgagcggcggacgggtgagtaacgcgtgggtaacctgcctcatacagggggataacagtt |
| | | agaaatgactgctaataccgcataagaccacagcaccgcatggtgcgggggtaaaaactccggtggtatgagatggacccgc |
| | | gtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtgggagcatgtggtttaattcgaagcaacgcgaagaaccttacctgctcttgacatccc |
| | | gctgaccggacggtaatgcgtccttcccttcgggcagcggagacaggtggtgcatggttgtcgtcagct |
| Ruminococcus_ | SEQ ID | aacgagagtttgatcctggctcaggatgaacgctggcggcgtgcctaacacatgcaagtcgagcgaagcactttgcttagattc |
| torques | NO: 57 | ttcggatgaagaggattgtgactgagcggcggacgggtgagtaacgcgtgggtaacctgcctcatacagggggataacagtt |
| | | agaaatgactgctaataccgcataagaccacagcaccgcatggtgcgggggtaaaaactccggtggtatgagatggacccgc |
| | | gtctgattagctagttggtaaggtaacggcttaccaaggcgacgatcagtagccgacctgagagggtggccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccaggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatcccg |
| | | ctgaccggacggtaatgcgtccttcccttcggggcagcggagacaggtggtgcatggttgtcgtcagctcgtgtcgtgagatgt |
| | | tgggttaagtcccgcaacgagcgcaacccctatctttagtagccagcggccaggccgggcactctagagagactgccgggg |
| | | ataacccggaggaaggtggggatgacgtcaaatcatcatgccccttatgagcagggctacacacgtgctacaatggcgtaaa |
| | | caaagggaagcgagaccgcgaggtggagcaaatcccaaaaataacgtctcagttcggattgtagtctgcaactcgactacatg |
| | | aagctggaatcgctagtaatcgcgaatcagaatgtcgcggtgaatacgttcccgggtcttgtacacaccgcccgtcacaccatg |
| | | ggagtcagtaacgcccgaagtcagtgacccaaccgtaaggagggagctgccgaaggcgggaccgataactgggggtgaa |
| | | gtcgtaacaaggtagccgtatcggaaggtgcggctggatcacctccttt |
| Ruminococcus_ | SEQ ID | aacgagagtttgatcctggctcaggatgaacgctggcggcgtgcctaacacatgcaagtcgagcgaagcactttgcttagattc |
| torques | NO: 58 | ttcggatgaagaggattgtgactgagcggcggacgggtgagtaacgcgtgggtaacctgcctcatacagggggataacagtt |
| | | agaaatgactgctaataccgcataagaccacagcaccgcatggtgcgggggtaaaaactccggtggtatgagatggacccgc |
| | | gtctgattagctggttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctgggggagtacgttcgcaagaatgaaactcaaagg |
| | | aattgacgggggacccgcacaagcggtggagcatgtggtttaattcgaagcaacgcgaagaaccttacctgctcttgacatcc |
| | | cgctgaccggacggtaatgcgtccttcccttcggggcagcggagacaggtggtgcatggttgtcgtcagctcgtgtcgtgaga |
| | | tgttgggttaagtcccgcacgagcgcaaccctatctttagtagcagcggccaggccgggcactctaaaagagactgccgggg |
| | | ataatcccgggaggaaggtggggat |
| Ruminococcus_ | SEQ ID | tacgagagtttgatcctggctcaggatgaacgcgggcggcgtgcctaacacatgcaagtcgagcgaagcactttgcttagattc |
| torques | NO: 59 | ttcggatgaagaggattgtgactgagcggcggacgggtgagtaacgcgtgggtaacctgcctcatacagggggataacagtt |
| | | agaaatgactgctaataccgcataagaccacagcaccgcatggtgcgggggtaaaaactccggtggtatgagatggacccgc |
| | | gtctgattagctagttggtaaggtaacggcttaccaaggcgacgatcagtagccgacctgagagggtggccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatcccg |
| | | ctgaccggacggtaatgcgtccttcccttcggggcagcggagacaggtggtgcatggttgtcgtcagctcgtgtcgtgagatgt |
| | | tgggttaagtcccgcaacgagcgcaacccctatctttagtagccagcggccaggccgggcactctagagagactgccgggg |
| | | ataacccggaggaaggtggggatgacgtcaaatcatcatgccccttatgagcagggctacacacgtgctacaatggcgtaaa |
| | | caaagggaagcgagaccgcgaggtggagcaaatcccaaaaataacgtctcagttcggattgtagtctgcaactcgactacatg |
| | | aagctggaatcgctagtaatcgcgaatcagaatgtcgcggtgaatacgttcccgggtcttgtacacaccgcccgtcacaccatg |
| | | ggagtcagtaacgcccgaagtcagtgacccaaccgtaaggagggagctgccgaaggcgggaccgataactggggtgaagt |
| | | cgtaacaaggtagccgtatcggaaggtgcggctggatcacctccttt |
| Dorea_ | SEQ ID | aacgagagtttgatcctggctcaggatgaacgctggcggcgtgcttaacacatgcaagtcgagcgaagcgcttaagtttgattc |
| longicatena | NO: 60 | ttcggatgaagacttttgtgactgagcggcggacgggtgagtaacgcgtgggtaacctgcctcatacagggggataacagtta |
| | | gaaatgactgctaataccgcataagaccacggtaccgcatggtacagtggtaaaaactccggtggtatgagatggacccgcgt |
| | | ctgattaggtagttggtggggtaacggcctaccaagccgacgatcagtagccgacctgagagggtgaccggccacattggga |
| | | ctgagacacggcccagactcctacgggaggcagcagtggggaatattgcacaatggaggaaactctgatgcagcgacgcc |
| | | gcgtgaaggatgaagtatttcggtatgtaaacttctatcagcagggaagaaaatgacggtacctgactaagaagccccggcta |
| | | actacgtgccagcagccgcggtaatacgtagggggcaagcgttatccggatttactgggtgtaaagggagcgtagacggcac |
| | | ggcaagccagatgtgaaagcccggggctcaaccccgggactgcatttggaactgctgagctagagtgtcggagaggcaagt |
| | | ggaattcctagtgtagcggtgaaatgcgtagatattaggaggaacaccagtggcgaaggcggcttgctggacgatgactgac |
| | | gttgaggctcgaaagcgtggggagcaaacaggattagataccctggtagtccacgccgtaaacgatgactgctaggtgtcgg |
| | | gtggcaaagccattcggtgccgcagctaacgcaataagcagtccacctggggagtacgttcgcaagaatgaaactcaaagga |
| | | attgacggggacccgcacaagcggtggagcatgtggtttaattcgaagcaacgcgaagaaccttacctgatcttgacatcccg |
| | | atgaccgcttcgtaatggaagtttttcttcggaacatcggtgacaggtggtgcatggttgtcgtcagctcgtgtcgtgagatgttg |
| | | ggttaagtcccgcaacgagcgcaacccctatcttcagtagccagcaggttaagctgggcactctggagagactgccagggat |
| | | aacctggaggaaggtggggatgacgtcaaatcatcatgccccttatgaccagggctacacacgtgctacaatggcgtaaacaa |
| | | agagaagcgaactcgcgagggtaagcaaatctcaaaaataacgtctcagttcggattgtagtctgcaactcgactacatgaagc |
| | | tggaatcgctagtaatcgcagatcagaatgctgcggtgaatacgttcccgggtcttgtacacaccgcccgtcacaccatggga |
| | | gtcagtaacgcccgaagtcagtgacccaaccgtaaggagggagagccgaaggtgggaccgataactggggtgaagtcgt |
| | | aacaaggtagccgtatcggaaggtgcggctggatcacctccttt |
| Dorea_ | SEQ ID | aacgagagtttgatcctggctcaggatgaacgctggcggcgtgcttaacacatgcaagtcgagcgaagcgctttggaaagatt |
| longicatena | NO: 61 | cttcggatgatttcctttgtgactgagcggcggacgggtgagtaacgcgtgggtaacctgcctcatacagggggataacagtta |
| | | gaaatgactgctaataccgcataagaccacggtaccgcatggtacagtggtaaaaactccggtggtatgagatggacccgcgt |
| | | ctgattaggtagttggtggggtaacggcctaccaagccgacgatcagtagccgacctgagagggtgaccggccacattggga |
| | | ctgagacacggcccagactcctacgggaggcagcagtggggaatattgcacaatggaggaaactctgatgcagcgacgcc |
| | | gcgtgaaggatgaagtatttcggtatgtaaacttctatcagcagggaagaaaatgacggtacctgactaagaagccccggcta |
| | | actacgtgccagcagccgcggtaatacgtagggggcaagcgttatccggatttactgggtgtaaagggagcgtagacggcac |
| | | ggcaagccagatgtgaaagcccggggctcaaccccgggactgcatttggaactgctgagctagagtgtcggagaggcaagt |
| | | ggaattcctagtgtagcggtgaaatgcgtagatattaggaggaacaccagtggcgaagcggcttgctggacgatgactgacgt |
| | | tgaggctcgaaagcgtggggagcaaacaggattagataccctggtagtccacgccgtaaacgatgactgctaggtgtcgggt |
| | | gg |
| Anaerostipes_ | SEQ ID | atgagagtttgatcctggctcaggatgaacgctggcggcgtgcttaacacatgcaagtcgaacgaagcatttaggattgaagttt |
| caccae | NO: 62 | tcggatggatttcctatatgactgagtggcggacgggtgagtaacgcgtggggaacctgccctatacagggggataacagctg |
| | | gaaacggctgctaataccgcataagcgcacagaatcgcatgattcagtgtgaaaagccctggcagtataggatggtcccgcgt |
| | | ctgattagctggttggtgaggtaacggctcaccaaggcgacgatcagtagccggcttgagagagtgaacggccacattggga |
| | | ctgagacacggcccaaactcctacgggaggcagcagtggggaatattgcacaatgggggaaaccctgatgcagcgacgcc |
| | | gcgtgagtgaagaagtatttcggtatgtaaagctctatcagcagggaagaaaacagacggtacctgactaagaagccccggct |
| | | aactacgtgccagcagccgcggtaatacgtagggggcaagcgttatccggaattactgggtgtaaagggtgcgtaggtggca |
| | | tggtaagtcagaagtgaaagcccggggcttaaccccgggactgcttttgaaactgtcatgctggagtgcaggagaggtaagc |
| | | ggaattcctagtgtagcggtgaaatgcgtagatattaggaggaacaccagtggcgaaggcggcttactggactgtcactgaca |
| | | ctgatgcacgaaagcgtggggagcaaacaggattagataccctggtagtccacgccgtaaacgatgaatactaggtgtcggg |
| | | gccgtagaggcttcggtgccgcagcaaacgcagtaagtattccacctggggagtacgttcgcaagaatgaaactcaaaggaa |
| | | ttgacggggacccgcacaagcggtggagcatgtggtttaattcgaagcaacgcgaagaaccttacctggtcttgacatcccaa |
| | | tgaccgaaccttaaccggttttttctttcgagacattggagacaggtggtgcatggttgtcgtcagctcgtgtcgtgagatgttggg |
| | | ttaagtcccgcaacgagcgcaacccctatctttagtagccagcattacggatgggcactctagagagactgccagggataacct |
| | | ggaggaaggtggggacgacgtcaaatcatcatgccccttatggccagggctacacacgtgctacaatggcgtaaacaaagg |
| | | gaagcgaagtcgtgaggcgaagcaaatcccagaaataacgtctcagttcggattgtagtctgcaactcgactacatgaagctg |
| | | gaatcgctagtaatcgtgaatcagaatgtcacggtgaatacgttcccgggtcttgtacacaccgcccgtcacaccatgggagtc |
| | | agtaacgcccgaagtcagtgacccaaccgcaaggagggagctgccgaaggtgggaccgataactggggtgaagtcgtaac |
| | | aaggtagccgtatcggaaggtgcggctggatcacctccttt |
| Anaerostipes_ | SEQ ID | ctagcggtgaatgcgtagatattaggaggaacaccagtggcgaaggcggcttactggactgtcactgacactgatgcacgaa |
| caccae | NO: 63 | agcgtggggagcaaacaggattagataccctggtagtccacgccgtaaacgatgaatactaggtgtcggggccgtagaggct |
| | | tcggtgccgcagcaaacgcagtaagtattccacctggggagtacgttcgcaagaatgaaactcaaaggaattgacggggacc |
| | | cgcacaagcggtggagcatgtggtttaattcgaagcaacgcgaagaaccttacctggtcttgacatcccaatgagcgaacctta |
| | | accggttttttctttcgagacattggagacaggtggtgcatggttgtcgtcagctcgtgtcgtgagatgttgggttaagtcccgcaa |
| | | cgagcgcaacccctatctttagtagccagcatttaaggtgggcactctagagagactgccagggataacctggaggaaggtg |
| | | gggacgacgtcaaatcatcatgccccttatggccagggctacacacgtgctacaatggcgtaaacaaagggaagcgaagtcg |
| | | tgaggcgaagcaaatcccagaaataacgtctcagttcggattgtagtctgcaactcgactacatgaagctggaatcgctagtaat |
| | | cgtgaatcagaatgtcacggtgaatacgttcccgggtcttgtacacaccgcccgtcacaccatgggagtcagtaacgcccgaa |
| | | gtcagtgacccaaccgcaaggagggagctgccgaaggtgggaccgataactggggtgaagtcgtaacaaggtagccgtat |
| | | cggaaggtgcggctggatcacctccttt |
| Anaerostipes_ | SEQ ID | atgagagtttgatcctggctcaggatgaacgctggcggcgtgcttaacacatgcaagtcgaacgaagcatttaggattgaagttt |
| caccae | NO: 64 | tcggatggatttcctatatgactgagtggcggacgggtgagtaacgcgtggggaacctgccctatacagggggataacagctg |
| | | gaaacggctgctaataccgcataagcgcacagaatcgcatgattcagtgtgaaaagccctggcagtataggatggtcccgcgt |
| | | ctgattagctggttggtgaggtaacggctcaccaaggcgacgatcagtagccggcttgagagagtgaacggccacattggga |
| | | ctgagacacggcccaaactcctacgggaggcagcagtggggaatattgcacaatgggggaaaccctgatgcagcgacgcc |
| | | gcgtgagtgaagaagtatttcggtatgtaaagctctatcagcagggaagaaaacagacggtacctgactaagaagccccggct |
| | | aactacgtgccagcagccgcggtaatacgtagggggcaagcgttatccggaattactgggtgtaaagggtgcgtaggtggca |
| | | tggtaagtcagaagtgaaagcccggggcttaaccccgggactgcttttgaaactgtcatgctggagtgcaggagaggtaagc |
| | | ggaattcctagtgtagcggtgaaatgcgtagatattaggaggaacaccagtggcgaaggcggcttactggactgtcactgaca |
| | | ctgatgcacgaaagcgtggggagcaaacaggattagataccctggtagtccacgccgtaaacgatgaatactaggtgtcggg |
| | | gccgtagaggcttcggtgccgcagcaaacgcagtaagtattccacctggggagtacgttcgcaagaatgaaactcaaaggaa |
| | | ttgacggggacccgcacaagcggtggagcatgtggtttaattcgaagcaacgcgaagaaccttacctggtcttgacatcccaa |
| | | tgaccgaaccttaaccggttttttctttcgagacattggagacaggtggtgcatggttgtcgtcagctcgtgtcgtgagatgttggg |
| | | ttaagtcccgcaacgagcgcaacccctatctttagtagccagcatttaaggtgggcactctagagagactgccagggataacct |
| | | ggaggaaggtggggacgacgtcaaatcatcatgccccttatggccagggctacacacgtgctacaatggcgtaaacaaagg |
| | | gaagcgaagtcgtgaggcgaagcaaatcccagaaataacgtctcagttcggattgtagtctgcaactcgactacatgaagctg |
| | | gaatcgctagtaatcgtgaatcagaatgtcacggtgaatacgttcccgggtcttgtacacaccgcccgtcacaccatgggagtc |
| | | agtaacgcccgaagtcagtgacccaaccgcaan |
| Anaerostipes_ | SEQ ID | atgagagtttgatcctggctcaggatgaacgctggcggcgtgcttaacacatgcaagtcgaacgaagcatttaggattgaagttt |
| caccae | NO: 65 | tcggatggatttcctatatgactgagtggcggacgggtgagtaacgcgtggggaacctgccctatacagggggataacagctg |
| | | gaaacggctgctaataccgcataagcgcacagaatcgcatgattcagtgtgaaaagccctggcagtataggatggtcccgcgt |
| | | ctgattagctggttggtgaggtaacggctcaccaaggcgacgatcagtagccggcttgagagagtgaacggccacattggga |
| | | ctgagacacggcccaaactcctacgggaggcagcagtggggaatattgcacaatgggggaaaccctgatgcagcgacgcc |
| | | gcgtgagtgaagaagtatttcggtatgtaaagctctatcagcagggaagaaaacagacggtacctgactaagaagccccggct |
| | | aactacgtgccagcagccgcggtaatacgtagggggcaagcgttatccggaattactgggtgtaaagggtgcgtaggtggca |
| | | tggtaagtcagaagtgaaagcccggggcttaaccccgggactgcttttgaaactgtcatgctggagtgcaggagaggtaagc |
| | | ggaattcctagtgtagcggtgaaatgcgtagatattaggaggaacaccagtggcgaaggcggcttactggactgtcactgaca |
| | | ctgatgcacgaaagcgtggggagcaaacaggattagataccctggtagtccacgccgtaaacgatgaatactaggtgtcggg |
| | | gccgtagaggcttcggtgccgcagcaaacgcagtaagtattccacctggggagtacgttcgcaagaatgaaactcaaaggaa |
| | | ttgacggggacccgcacaagcggtggagcatgtggtttaattcgaagcaacgcgaagaaccttacctggtcttgacatcccaa |
| | | tgaccgaaccttaaccggttttttctttcgagacattggagacaggtggtgcatggttgtcgtcagctcgtgtcgtgagatgttggg |
| | | ttaagtcccgcaacgagcgcaacccctatctttagtagccagcatttaaggtgggcactctagagagactgccagggataacct |
| | | ggaggaaggtggggacgacgtcaaatcatcatgccccttatggccagggctacacacgtgctacaatggcgtaaacaaagg |
| | | gaagcgaagtcgtgaggcgaagcaaatcccagaaataacgtctcagttcggattgtagtctgcaactcgactacatgaagctg |
| | | gaatcgctagtaatcgtgaatcagaatgtcacggtgaatacgttcccgggtcttgtacacaccgcccgtcacaccatgggagtc |
| | | agtaacgcccgaagtcagtgacccaaccgcaaggagggagctgccgaaggtgggaccgataactggggtgaagtcgtaac |
| | | aaggtagccgtatcggaaggtgcggctggatcacctccttt |
| Anaerostipes_ | SEQ ID | atgagagtttgatcctggctcaggatgaacgctggcggcgtgcttaacacatgcaagtcgaacgaagcatttaggattgaagttt |
| caccae | NO: 66 | tcggatggatttcctatatgactgagtggcggacgggtgagtaacgcgtggggaacctgccctatacaggggggataacagct |
| | | ggaaacggctgctaataccgcataagcgcacagaatcgcatgattcagtgtgaaaagccctggcagtataggatggtcccgc |
| | | gtctgattagctggttggtgaggtaacggctcaccaaggcgacgatcagtagccggcttgagagagtgaacggccacattgg |
| | | gactgagacacggcccaaactcctacgggaggcagcagtggggaatattgcacaatgggggaaaccctgatgcagcgacg |
| | | ccgcgtgagtgaagaagtatttcggtatgtaaagctctatcagcagggaagaaaacagacggtacctgactaagaagccccg |
| | | gctaactacgtgccagcagccgcggtaatacgtagggggcaagcgttatccggaattactgggtgtaaagggtgcgtaggtg |
| | | gcatggtaagtcagaagtgaaagcccggggcttaaccccgggactgcttttgaaactgtcatgctggagtgcaggagaggta |
| | | agcggaattcctagtgtagcggtgaaatgcgtagatattaggaggaacaccagtggcgaaggcggcttactggactgtcactg |
| | | acactgatgcacgaaagcgtggggagcaaacaggattagataccctggtagtccacgccgtaaacgatgaatactaggtgtc |
| | | ggggccgtagaggcttcggtgccgcagcaaacgcagtaagtattccacctggggagtacgttcgcaagaatgaaactcaaag |
| | | gaattgacggggacccgcacaagcggtggagcatgtggtttaattcgaagcaacgcgaagaaccttacctggtcttgacatcc |
| | | caatgagcgaaccttaaccggttttttctttcgagacattggagacaggtggtgcatggttgtcgtcagctcgtgtcgtgagatgtt |
| | | gggttaagtcccgcaacgagcgcaacccctatctttagtagccagcatttaaggtgggcactctagagagactgccagggata |
| | | acctggaggaaggtggggacgacgtcaaatcatcatgccccttatggccagggctacacacgtgctacaatggcgtaaacaa |
| | | agggaagcgaagtcgtgaggcgaagcaaatcccagaaataacgtctcagttcggattgtagtctgcaactcgactacatgaag |
| | | ctggaatcgctagtaatcgtgaatcagaatgtcacggtgaatacgttcccgggtcttgtacacaccgcccgtcacaccatggga |
| | | gtcagtaacgcccgaagtcagtgacccaaccgcaaggagggagctgccgaaggtgggaccgataactggggtgaagtcgt |
| | | aacaaggtagccgtatcggaaggtgcggctggatcacctccttt |
| Eubacterium_ | SEQ ID | caaagagtttgatcctggctcaggacgaacgctggcggcgcgcctaacacatgcaagtcgaacggtgaagaggagcttgctc |
| siraeum | NO: 67 | ctcggatcagtggcggacgggtgagtaacacgtgagcaacctggctctaagagggggacaacagttggaaacgactgctaa |
| | | taccgcataacgtatcgggatggcatcttcctgataccaaagattttatcgcttagagatgggctcgcgtctgattagatagttggc |
| | | ggggtaacggcccaccaagtcgacgatcagtagccggactgagaggttgaacggccacattgggactgagacacggccca |
| | | gactcctacgggaggcagcagtgggggatattggacaatgggggcaaccctgatccagcgacgccgcgtgagggaagaa |
| | | ggttttcggattgtaaacctctgttgacggagaaaaaaatgatggtatccgtttagaaagccacggctaactacgtgccagcagc |
| | | cgcggtaatacgtaggtggcaagcgttgtccggaattactgggtgtaaagggagtgtaggcgggatatcaagtcagaagtga |
| | | aaattacgggctcaactcgtaacctgcttttgaaactgacattcttgagtgaagtagaggcaagcggaattcctagtgtagcggt |
| | | gaaatgcgtagatattaggaggaacaccagtggcgaaggcggcttgctgggcttttactgacgctgaggctcgaaagcgtgg |
| | | ggagcaaacaggattagataccctggtagtccacgccgtaaacgatgattactaggtgtggggggattgaccccttccgtgcc |
| | | ggagtaaacacaataagtaatccacctggggagtacgaccgcaaggttgaaactcaaaggaattgacgggggcccgcacaa |
| | | gcagtggagtatgtggtttaattcgacgcaacgcgaagaaccttaccaggtcttgacatcgagtgaccgcctaagagattaggc |
| | | tttcccttcggggacacaaagacaggtggtgcatggttgtcgtcagctcgtgtcgtgagatgttgggttaagtcccgcaacgag |
| | | cgcaacccttatcattagttgctacgcaagagcactctaatgagactgccgttgacaaaacggaggaaggtggggatgacgtc |
| | | aaatcatcatgccctttatgacctgggctacacacgtactacaatggcgtttaacaaagagaagcaaagccgcgaggcagagc |
| | | aaatctccaaaaaacgtctcagttcggattgtaggctgcaactcgcctacatgaagtcggaattgctagtaatcgtaggtcagca |
| | | tactacggtgaatacgttcccgggccttgtacacaccgcccgtcaaaccatgagagttggcaacacccgaagtcggtagtctaa |
| | | ccgcaaggaggacgccgccgaaggtggggttgatgattagggttaagtcgtaacaaggtagccgtatcggaaggtgcggct |
| | | ggatcacctccttt |
| Eubacterium_ | SEQ ID | caaagagtttgatcctggctcaggacgaacgctggcggcgcgcctaacacatgcaagtcgaacggtgaagaggagcttgctc |
| siraeum | NO: 68 | ctcggaacagtggcggacgggtgagtaacacgtgagcaacctggctctaagagggggacaacagttggaaacgactgctaa |
| | | taccgcataacgtatcgggatggcatcttcctgataccaaagattttatcgcttagagatgggctcgcgtctgattagatagttggc |
| | | ggggtaacggcccaccaagtcgacgatcagtagccggactgagaggttgaacggccacattgggactgagacacggccca |
| | | gactcctacgggaggcagcagtgggggatattggacaatgggggcaaccctgatccagcgacgccgcgtgagggaagaa |
| | | ggttttcggattgtaaacctctgttgacggagaaaaaaatgatggtatccgtttagaaagccacggctaactacgtgccagcagc |
| | | cgcggtaatacgtaggtggcaagcgttgtccggaattactgggtgtaaagggagtgtaggcgggatatcaagtcagaagtga |
| | | aaattacgggctcaactcgtaacctgcttttgaaactgacattcttgagtgaagtagaggcaagcggaattcctagtgtagcggt |
| | | gaaatgcgtagatattaggaggaacaccagtggcgaaggcggcttgctgggcttttactgacgctgaggctcgaaagcgtgg |
| | | ggagcaaacaggattagataccctggtagtccacgccgtaaacgatgattactaggtgtggggggattgaccccttccgtgcc |
| | | ggagtaaacacaataagtaatccacctggggagtacgaccgcaaggttgaaactcaaaggaattgacgggggcccgcacaa |
| | | gcagtggagtatgtggtttaattcgacgcaacgcgaagaaccttaccaggtcttgacatcgagtgaccgcctaagagattaggc |
| | | tttcccttcggggacacaaagacaggtggtgcatggttgtcgtcagctcgtgtcgtgagatgttgggttaagtcccgcaacgag |
| | | cgcaacccttatcattagttgctacgcaagagcactctaatgagactgccgttgacaaaacggaggaaggtggggatgacgtc |
| | | aaatcatcatgccctttatgacctgggctacacacgtactacaatggcgtttaacaaagagaagcaaagccgcgaggcagagc |
| | | aaatctccaaaaaacgtctcagttcggattgtaggctgcaactcgcctacatgaagtcggaattgctagtaatcgtaggtcagca |
| | | tactacggtgaatacgttcccgggccttgtacacaccgcccgtcaaaccatgagagttggcaacacccgaagtcggtagtctaa |
| | | ccgcaaggaggacgccgccgaaggtggggttgatgattagggttaagtcgtaacaaggtagccgtatcggaaggtgcggct |
| | | ggatcacctccttt |
| Eubacterium_ | SEQ ID | caaagagtttgatcctggctcaggacgaacgctggcggcgcgcctaacacatgcaagtcgaacggtgaagaggagcttgctc |
| siraeum | NO: 69 | ctcggatcagtggcggacgggtgagtaacacgtgagcaacctggctctaagagggggacaacagttggaaacgactgctaa |
| | | taccgcatgatgtatcgggatggcatcttcctgataccaaagattttatcgcttagagatgggctcgcgtctgattagatagttggc |
| | | ggggtaacggcccaccaagtcgacgatcagtagccggactgagaggttgaacggccacattgggactgagacacggccca |
| | | gactcctacgggaggcagcagtgggggatattggacaatgggggcaaccctgatccagcgacgccgcgtgagggaagaa |
| | | ggttttcggattgtaaacctctgttgacggagaaaaaaatgatggtatccgtttagaaagccacggctaactacgtgccagcagc |
| | | cgcggtaatacgtaggtggcaagcgttgtccggaattactgggtgtaaagggagtgtaggcgggatatcaagtcagaagtga |
| | | aaattacgggctcaactcgtaacctgcttttgaaactgacattcttgagtgaagtagaggcaagcggaattcctagtgtagcggt |
| | | gaaatgcgtagatattaggaggaacaccagtggcgaaggcggcttgctgggcttttactgacgctgaggctcgaaagcgtgg |
| | | ggagcaaacaggattagataccctggtagtccacgccgtaaacgatgattactaggtgtggggggattgaccccttccgtgcc |
| | | ggagtaaacacaataagtaatccacctggggagtacgaccgcaaggttgaaactcaaaggaattgacgggggcccgcacaa |
| | | gcagtggagtatgtggtttaattcgacgcaacgcgaagaaccttaccaggtcttgacatcgagtgaccgcctaagagattaggc |
| | | tttcccttcggggacacaaagacaggtggtgcatggttgtcgtcagctcgtgtcgtgagatgttgggttaagtcccgcaacgag |
| | | cgcaacccttatcattagttgctacgcaagagcactctaatgagactgccgttgacaaaacggaggaaggtggggatgacgtc |
| | | aaatcatcatgccctttatgacctgggctacacacgtactacaatggcgtttaacaaagagaagcaaagccgcgaggcagagc |
| | | aaatctccaaaaaacgtctcagttcggattgtaggctgcaactcgcctacatgaagtcggaattgctagtaatcgtaggtcagca |
| | | tactacggtgaatacgttcccgggccttgtacacaccgcccgtcaaaccatgagagttggcaacacccgaagtcggtagtctaa |
| | | ccgcaaggaggacgccgccgaaggtggggttgatgattagggttaagtcgtaacaaggtagccgtatcggaaggtgcggct |
| | | ggatcacctccttt |
| Eubacterium_ | SEQ ID | caaagagtttgatcctggctcaggacgaacgctggcggcgcgcctaacacatgcaagtcgaacggtgaagaggagcttgctc |
| siraeum | NO: 70 | ctcggatcagtggcggacgggtgagtaacacgtgagcaacctggctctaagagggggacaacagttggaaacgactgctaa |
| | | taccgcataacgtatcgggatggcatcttcctgataccaaagattttatcgcttagagatgggctcgcgtctgattagatagttggc |
| | | ggggtaacggcccaccaagtcgacgatcagtagccggactgagaggttgaacggccacattgggactgagacacggccca |
| | | gactcctacgggaggcagcagtgggggatattggacaatgggggcaaccctgatccagcgacgccgcgtgagggaagaa |
| | | ggttttcggattgtaaacctctgttgacggagaaaaaaatgatggtatccgtttagaaagccacggctaactacgtgccagcagc |
| | | cgcggtaatacgtaggtggcaagcgttgtccggaattactgggtgtaaagggagtgtaggcgggatatcaagtcagaagtga |
| | | aaattacgggctcaactcgtaacctgcttttgaaactgacattcttgagtgaagtagaggcaagcggaattcctagtgtagcggt |
| | | gaaatgcgtagatattaggaggaacaccagtggcgaaggcggcttgctgggcttttactgacgctgaggctcgaaagcgtgg |
| | | ggagcaaacaggattagataccctggtagtccacgccgtaaacgatgattactaggtgtggggggattgaccccttccgtgcc |
| | | ggagtaaacacaataagtaatccacctggggagtacgaccgcaaggttgaaactcaaaggaattgacgggggcccgcacaa |
| | | gcagtggagtatgtggtttaattcgacgcaacgcgaagaaccttaccaggtcttgacatcgagtgaccgcctaagagattaggc |
| | | cttcccttcggggacacaaagacaggtggtgcatggttgtcgtcagctcgtgtcgtgagatgttgggttaagtcccgcaacgag |
| | | cgcaacccttatcattagttgctacgcaagagcactctaatgagactgccgttgacaaaacggaggaaggtggggatgacgtc |
| | | aaatcatcatgccctttatgacctgggctacacacgtactacaatggcgtttaacaaagagaagcaaagccgcgaggcagagc |
| | | aaatctccaaaaaacgtctcagttcggattgtaggctgcaactcgcctacatgaagtcggaattgctagtaatcgtaggtcagca |
| | | tactacggtgaatacgttcccgggccttgtacacaccgcccgtcaaaccatgagagttggcaacacccgaagtcggtagtctaa |
| | | ccgcaaggaggacgccgccgaaggtggggttgatgattagggttaagtcgtaacaaggtagccgtatcggaaggtgcggct |
| | | ggatcacctccttt |
| Clostridium_ | SEQ ID | tttagagagtttgatcctggctcaggacgaacgctggcggcgtgcctaacacatgcaagtcgaacggagttaaattcgacacc |
| leptum | NO: 71 | cgagtatccggccgggaggcggggtgctgggggttggatttaacttagtggcggacgggtgagtaacgcgtgagtaacctg |
| | | cctttcagagggggataacgttctgaaaagaacgctaataccgcataacatcaatttatcgcatgataggttgatcaaaggagca |
| | | atccgctggaagatggactcgcgtccgattagccagttggcggggtaacggcccaccaaagcgacgatcggtagccggact |
| | | gagaggttgaacggccacattgggactgagacacggcccagactcctacgggaggcagcagtgggggatattgcacaatg |
| | | ggggaaaccctgatgcagcaacgccgcgtgagggaagaaggttttcggattgtaaacctctgttcttagtgacgataatgacg |
| | | gtagctaaggagaaagctccggctaactacgtgccagcagccgcggtaatacgtagggagcgagcgttgtccggatttactg |
| | | ggtgtaaagggtgcgtaggcggcgaggcaagtcaggcgtgaaatctatgggcttaacccataaactgcgcttgaaactgtctt |
| | | gcttgagtgaagtagaggtaggcggaattcccggtgtagcggtgaaatgcgtagagatcgggaggaacaccagtggcgaag |
| | | gcggcctactgggctttaactgacgctgaagcacgaaagcatgggtagcaaacaggattagataccctggtagtccatgccgt |
| | | aaacgatgattactaggtgtggggggtctgaccccctccgtgccgcagttaacacaataagtaatccacctggggagtacggc |
| | | cgcaaggttgaaactcaaaggaattgacgggggcccgcacaagcagtggagtatgtggtttaattcgaagcaacgcgaagaa |
| | | ccttaccaggtcttgacatccgtctaacgaagcagagatgcattaggtgcccttcggggaaaggcgagacaggtggtgcatgg |
| | | ttgtcgtcagctcgtgtcgtgagatgttgggttaagtcccgcaacgagcgcaacccttgtttctagttgctacgcaagagcactct |
| | | agagagactgccgttgacaaaacggaggaaggtggggacgacgtcaaatcatcatgccccttatgacctgggccacacacgt |
| | | actacaatggctgtaaacagagggaagcaaagccgcgaggtggagcaaaaccctaaaagcagtcccagttcggatcgcag |
| | | gctgcaacccgcctgcgtgaagtcggaattgctagtaatcgcggatcagcatgccgcggtgaatacgttcccgggccttgtac |
| | | acaccgcccgtcacaccatgggagccggtaatacccgaagccagtagttcaaccgcaaggagagcgctgtcgaaggtagg |
| | | attggcgactggggtgaagtcgtaacaaggtagccgtatcggaaggtgcggaggatcacctccttt |
| Clostridium_ | SEQ ID | tttagagagtttgatcctggctcaggacgaacgctggcggcgtgcctaacacatgcaagtcgaacggagttaaattcgacacc |
| leptum | NO: 72 | cgagtatccggccgggaggcggggtgctgggggttggatttaacttagtggcggacgggtgagtaacgcgtgagtaacctg |
| | | cctttcagagggggataacgttctgaaaagaacgctaataccgcataacatcaatttatcgcatgataggttgatcaaaggagca |
| | | atccgctggaagatggactcgcgtccgattagccagttggcggggtaacggcccaccaaagcgacgatcggtagccggact |
| | | gagaggttgaacggccacattgggactgagacacggcccagactcctacgggaggcagcagtgggggatattgcacaatg |
| | | ggggaaaccctgatgcagcaacgccgcgtgagggaagaaggttttcggattgtaaacctctgttcttagtgacgataatgacg |
| | | gtagctaaggagaaagctccggctaactacgtgccagcagccgcggtaatacgtagggagcgagcgttgtccggatttactg |
| | | ggtgtaaagggtgcgtaggcggcgaggcaagtcaggcgtgaaatctatgggcttaacccataaactgcgcttgaaactgtctt |
| | | gcttgagtgaagtagaggtaggcggaattcccggtgtagcggtgaaatgcgtagagatcgggaggaacaccagtggcgaag |
| | | gcggcctactgggctttaactgacgctgaagcacgaaagcatgggtagcaaacaggattagataccctggtagtccatgccgt |
| | | aaacgatgattactaggtgtggggggtctgaccccctccgtgccgcagttaacacaataagtaatccacctggggagtacggc |
| | | cgcaaggttgaaactcaaaggaattgacgggggcccgcacaagcagtggagtatgtggtttaattcgaagcaacgcgaagaa |
| | | ccttaccaggtcttgacatccgtctaacgaagcagagatgcattaggtgcccttcggggaaaggcgagacaggtggtgcatgg |
| | | ttgtcgtcagctcgtgtcgtgagatgttgggttaagtcccgcaacgagcgcaacccttgtttctagttgctacgcaagagcactct |
| | | agagagactgccgttgacaaaacggaggaaggtggggacgacgtcaaatcatcatgccccttatgacctgggccacacacgt |
| | | actacaatggctgtaaacagagggaagcaaagccgcgaggtggagcaaaaccctaaaagcagtcccagttcggatcgcag |
| | | gctgcaacccgcctgcgtgaagtcggaattgctagtaatcgcggatcagcatgccgcggtgaatacgttcccgggccttgtac |
| | | acaccgcccgtcacaccatgggagccggtaatacccgaagccagtagttcaaccgcaaggagagcgctgtcgaaggtagg |
| | | attggcgactggggtgaagtcgtaacaaggtagccgtatcggaaggtgcggaggatcacctccttt |
| Clostridium_ | SEQ ID | atgagagtttgatcctggctcaggatgaacgctggcggcgtgcctaacacatgcaagtcgaacgaagcaattgaaggaagtttt |
| boltcae | NO: 73 | cggatggaattcgattgactgagtggcggacgggtgagtaacgcgtggataacctgcctcacactgggggataacagttaga |
| | | aatgactgctaataccgcataagcgcacagtaccgcatggtacagtgtgaaaaactccggtggtgtgagatggatccgcgtct |
| | | gattagccagaggcggggtaacggcccaccaaagcgacgatcagtagccgacctgagagggtgaccggccacattggga |
| | | ctgagacacggcccaaactcctacgggaggcagcagtggggaatattgcacaatgggcgaaagcctgatgcagcgacgcc |
| | | gcgtgagtgaagaagtatttcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgtagacggcg |
| | | aagcaagtctgaagtgaaaacccagggctcaaccctgggactgctttggaaactgttttgctagagtgtcggagaggtaagtg |
| | | gaattcctagtgtagcggtgaaatgcgtagatattaggaggaacaccagtggcgaaggcggcttactggacgataactgacgt |
| | | tgaggctcgaaagcgtggggagcaaacaggattagataccctggtagtccacgccgtaaacgatgaatgctaggtgttgggg |
| | | ggcaaagcccttcggtgccgtcgcaaacgcagtaagcattccacctggggagtacgttcgcaagaatgaaactcaaaggaatt |
| | | gacggggacccgcacaagcggtggagcatgtggtttaattcgaagcaacgcgaagaaccttaccaagtcttgacatcctcttg |
| | | accggcgtgtaacggcgccttcccttcggggcaagagagacaggtggtgcatggttgtcgtcagctcgtgtcgtgagatgttg |
| | | ggttaagtcccgcaacgagcgcaacccttatccttagtagccagcaggtaaagctgggcactctagggagactgccagggat |
| | | aacctggaggaaggtggggatgacgtcaaatcatcatgccccttatgatttgggctacacacgtgctacaatggcgtaaacaaa |
| | | gggaagcaagacagtgatgtggagcaaatcccaaaaataacgtcccagttcggactgtagtctgcaacccgactacacgaag |
| | | ctggaatcgctagtaatcgcgaatcagaatgtcgcggtgaatacgttcccgggtcttgtacacaccgcccgtcacaccatggga |
| | | gtcagcaacgcccgaagtcagtgacccaactcgcaagagagggagctgccgaaggcggggcaggtaactggggtgaagt |
| | | cgtaacaaggtagccgtatcggaaggtgcggctggatcacctccttt |
| Faecalibacterium_ | SEQ ID | tataaagagtttgatcctggctcaggacgaacgctggcggcgcgcctaacacatgcaagtcgaacgagcgagagagagcttg |
| prausnitzii | NO: 74 | ctttcttgagcgagtggcgaacgggtgagtaacgcgtgaggaacctgcctcaaagagggggacaacagttggaaacgactg |
| | | ctaataccgcataagcccacggctcggcatcgagcagagggaaaaggagcaatccgctttgagatggcctcgcgtccgatta |
| | | gctggttggtgaggtaacggcccaccaaggcgacgatcggtagccggactgagaggttgaacggccacattgggactgaga |
| | | cacggcccagactcctacgggaggcagcagtggggaatattgcacaatgggggaaaccctgatgcagcgacgccgcgtgg |
| | | aggaagaaggtcttcggattgtaaactcctgagttgaggaagataatgacggtactcaacaaggaagtgacggctaactacgt |
| | | gccagcagccgcggtaaaacgtaggtcacaagcgttgtccggaattactgggtgtaaagggagcgcaggcgggaagacaa |
| | | gttggaagtgaaatccatgggctcaacccatgaactgctttcaaaactgtttttcttgagtagtgcagaggtaggcggaattcccg |
| | | gtgtagcggtggaatgcgtagatatcgggaggaacaccagtggcgaaggcggcctactgggcaccaactgacgctgaggct |
| | | cgaaagtgtgggtagcaaacaggattagataccctggtagtccacactgtaaacgatgattactaggtgttggaggattgaccc |
| | | cttcagtgccgcagttaacacaataagtaatccacctggggagtacgaccgcaaggttgaaactcaaaggaattgacggggg |
| | | cccgcacaagcagtggagtatgtggtttaattcgacgcaacgcgaagaaccttaccaagtcttgacatcccttgacgatgctgg |
| | | aaacagtatttctcttcggagcaaggagacaggtggtgcatggttgtcgtcagctcgtgtcgtgagatgttgggttaagtcccgc |
| | | aacgagcgcaacccttatggtcagttactacgcaagaggactctggccagactgccgttgacaaaacggaggaaggtgggg |
| | | atgacgtcaaatcatcatgccctttatgacttgggctacacacgtactacaatggcgttaaacaaagagaagcaagaccgcgag |
| | | gtggagcaaaactcagaaacaacgtcccagttcggactgcaggctgcaactcgcctgcacgaagtcggaattgctagtaatcg |
| | | cagatcagcatgctgcggtgaatacgttcccgggccttgtacacaccgcccgtcacaccatgagagccggggggacccgaa |
| | | gtcggtagtctaaccgcaaggaggacgccgccgaaggtaaaactggtgattggggtgaagtcgtaacaaggtagccgtagg |
| | | agaacctgcggctggatcacctccttt |
| Faecalibacterium_ | SEQ ID | tataaagagtttgatcctggctcaggacgaacgctggcggcgcgcctaacacatgcaagtcgaacgagcgagaggaggttta |
| prausnitzii | NO: 75 | ctttctcaagcgagtggcgaacgggtgagtaacgcgtgaggaacctgcctcaaagagggggacaacagttggaaacgactg |
| | | ctaataccgcataagcccacgacccggcatcgggtagagggaaaaggagcaatccgctttgagatggcctcgcgtccgatta |
| | | gctagttggtgaggtaatggcccaccaaggcgacgatcggtagccggactgagaggttgaacggccacattgggactgaga |
| | | cacggcccagactcctacgggaggcagcagtggggaatattgcacaatgggggaaaccctgatgcagcgacgccgcgtgg |
| | | aggaagaaggtcttcggattgtaaactcctgttgttgaggaagataatgacggtactcaacaaggaagtgacggctaactacgt |
| | | gccagcagccgcggtaaaacgtaggtcacaagcgttgtccggaattactgggtgtaaagggagcgcaggcgggaagacaa |
| | | gttggaagtgaaatccatgggctcaacccatgaactgctttcaaaactgtttttcttgagtagtgcagaggtaggcggaattcccg |
| | | gtgtagcggtggaatgcgtagatatcgggaggaacaccagtggcgaaggcggcctactgggcaccaactgacgctgaggct |
| | | cgaaagtgtgggtagcaaacaggattagataccctggtagtccacactgtaaacgatgattactaggtgttggaggattgaccc |
| | | cttcagtgccgcagttaacacaataagtaatccacctggggagtacgaccgcaaggttgaaactcaaaggaattgacggggg |
| | | cccgcacaagcagtggagtatgtggtttaattcgacgcaacgcgaagaaccttaccaagtcttgacatcctgcgacgcacatag |
| | | aaatatgtgtttccttcgggacgcagagacaggtggtgcatggttgtcgtcagctcgtgtcgtgagatgttgggttaagtcccgc |
| | | aacgagcgcaacccttatggtcagttactacgcaagaggactctggccagactgccgttgacaaaacggaggaaggtgggg |
| | | atgacgtcaaatcatcatgccctttatgacttgggctacacacgtactacaatggcgttaaacaaagagaagcaagaccgcgag |
| | | gtggagcaaaactcagaaacaacgtcccagttcggactgcaggctgcaactcgcctgcacgaagtcggaattgctagtaatcg |
| | | cagatcagcatgctgcggtgaatacgttcccgggccttgtacacaccgcccgtcacaccatgagagccggggggacccgaa |
| | | gtcggtagtctaaccgcaaggaggacgccgccgaaggtaaaactggtgattggggtgaagtcgtaacaaggtagccgtagg |
| | | agaacctgcggctggatcacctccttt |
| Faecalibacterium_ | SEQ ID | tataaagagtttgatcctggctcaggacgaacgctggcggcgcgcctaacacatgcaagtcgaacgagcgagagagagcttg |
| prausnitzii | NO: 76 | ctttctcaagcgagtggcgaacgggtgagtaacgcgtgaggaacctgcctcaaagagggggacaacagttggaaacgactg |
| | | ctaataccgcataagcccacgacccggcatcgggtagagggaaaaggagcaatccgctttgagatggcctcgcgtccgatta |
| | | gctagttggtgaggtaacggcccaccaaggcgacgatcggtagccggactgagaggttgaacggccacattgggactgaga |
| | | cacggcccagactcctacgggaggcagcagtggggaatattgcacaatgggggaaaccctgatgcagcgacgccgcgtgg |
| | | aggaagaaggtcttcggattgtaaactcctgttgttgaggaagataatgacggtactcaacaaggaagtgacggctaactacgt |
| | | gccagcagccgcggtaaaacgtaggtcacaagcgttgtccggaattactgggtgtaaagggagcgcaggcgggaagacaa |
| | | gttggaagtgaaatccatgggctcaacccatgaactgctttcaaaactgtttttcttgagtagtgcagaggtaggcggaattcccg |
| | | gtgtagcggtggaatgcgtagatatcgggaggaacaccagtggcgaaggcggcctactgggcaccaactgacgctgaggct |
| | | cgaaagtgtgggtagcaaacaggattagataccctggtagtccacaccgtaaacgatgattactaggtgttggaggattgaccc |
| | | cttcagtgccgcagttaacacaataagtaatccacctggggagtacgaccgcaaggttgaaactcaaaggaattgacggggg |
| | | cccgcacaagcagtggagtatgtggtttaattcgacgcaacgcgaagaaccttaccaagtcttgacatcccttgacgatgctgg |
| | | aaacagtatttctcttcggagcaaggagacaggtggtgcatggttgtcgtcagctcgtgtcgtgagatgttgggttaagtcccgc |
| | | aacgagcgcaacccttatggtcagttactacgcaagaggactctggccagactgccgttgacaaaacggaggaaggtgggg |
| | | atgacgtcaaatcatcatgccctttatgacttgggctacacacgtactacaatggcgttaaacaaagagaagcaagaccgcgag |
| | | gtggagcaaaactcagaaacaacgtcccagttcggactgcaggctgcaactcgcctgcacgaagtcggaattgctagtaatcg |
| | | cagatcagcatgctgcggtgaatacgttcccgggccttgtacacaccgcccgtcacaccatgagagccggggggacccgaa |
| | | gtcggtagtctaaccgcaaggaggacgccgccgaaggtaaaactggtgattggggtgaagtcgtaacaaggtagccgtagg |
| | | agaacctgcggctggatcacctccttt |
| Faecalibacterium_ | SEQ ID | tataaagagtttgatcctggctcaggacgaacgctggcggcgcgcctaacacatgcaagtcgaacgagcgagagagagcttg |
| prausnitzii | NO: 77 | ctttctcaagcgagtggcgaacgggtgagtaacgcgtgaggaacctgcctcaaagagggggacaacagttggaaacgactg |
| | | ctaataccgcataagcccacgacccggcatcgggtagagggaaaaggagcaatccgctttgagatggcctcgcgtccgatta |
| | | gctggttggtgaggtaacggcccaccaaggcgacgatcggtagccggactgagaggttgaacggccacattgggactgaga |
| | | cacggcccagactcctacgggaggcagcagtggggaatattgcacaatgggggaaaccctgatgcagcgacgccgcgtgg |
| | | aggaagaaggtcttcggattgtaaactcctgttgttgaggaagataatgacggtactcaacaaggaagtgacggctaactacgt |
| | | gccagcagccgcggtaaaacgtaggtcacaagcgttgtccggaattactgggtgtaaagggagcgcaggcgggaagacaa |
| | | gttggaagtgaaatccatgggctcaacccatgaactgctttcaaaactgtttttcttgagtagtgcagaggtaggcggaattcccg |
| | | gtgtagcggtggaatgcgtagatatcgggaggaacaccagtggcgaaggcggcctactgggcaccaactgacgctgaggct |
| | | cgaaagtgtgggtagcaaacaggattagataccctggtagtccacaccgtaaacgatgattactaggtgttggaggattgaccc |
| | | cttcagtgccgcagttaacacaataagtaatccacctggggagtacgaccgcaaggttgaaactcaaaggaattgacggggg |
| | | cccgcacaagcagtggagtatgtggtttaattcgacgcaacgcgaagaaccttaccaagtcttgacatcctgcgacgcacatag |
| | | aaatatgtgtttccttcgggacgcagagacaggtggtgcatggttgtcgtcagctcgtgtcgtgagatgttgggttaagtcccgc |
| | | aacgagcgcaacccttatggtcagttactacgcaagaggactctggccagactgccgttgacaaaacggaggaaggtgggg |
| | | atgacgtcaaatcatcatgccctttatgacttgggctacacacgtactacaatggcgttaaacaaagagaagcaagaccgcgag |
| | | gtggagcaaaactcagaaacaacgtcccagttcggactgcaggctgcaactcgcctgcacgaagtcggaattgctagtaatcg |
| | | cagatcagcatgctgcggtgaatacgttcccgggccttgtacacaccgcccgtcacaccatgagagccggggggacccgaa |
| | | gtcggtagtctaaccgcaaggaggacgccgccgaaggtaaaactggtgattggggtgaagtcgtaacaaggtagccgtagg |
| | | agaacctgcggctggatcacctccttt |
| Faecalibacterium_ | SEQ ID | tataaagagtttgatcctggctcaggacgaacgctggcggcgcgcctaacacatgcaagtcgaacgagcgagagagagcttg |
| prausnitzii | NO: 78 | ctttctcaagcgagtggcgaacgggtgagtaacgcgtgaggaacctgcctcaaagagggggacaacagttggaaacgactg |
| | | ctaataccgcataagcccacgacccggcatcgggtagagggaaaaggagcaatccgctttgagatggcctcgcgtccgatta |
| | | gctagttggtgaggtaacggcccaccaaggcgacgatcggtagccggactgagaggttgaacggccacattgggactgaga |
| | | cacggcccagactcctacgggaggcagcagtggggaatattgcacaatgggggaaaccctgatgcagcgacgccgcgtgg |
| | | aggaagaaggtcttcggattgtaaactcctgttgttgaggaagataatgacggtactcaacaaggaagtgacggctaactacgt |
| | | gccagcagccgcggtaaaacgtaggtcacaagcgttgtccggaattactgggtgtaaagggagcgcaggcgggaagacaa |
| | | gaggaagtgaaatccatgggctcaacccatgaactgctttcaaaactgtttttcttgagtagtgcagaggtaggcggaattcccg |
| | | gtgtagcggtggaatgcgtagatatcgggaggaacaccagtggcgaaggcggcctactgggcaccaactgacgctgaggct |
| | | cgaaagtgtgggtagcaaacaggattagataccctggtagtccacactgtaaacgatgattactaggtgttggaggattgaccc |
| | | cttcagtgccgcagttaacacaataagtaatccacctggggagtacgaccgcaaggttgaaactcaaaggaattgacggggg |
| | | cccgcacaagcagtggagtatgtggtttaattcgacgcaacgcgaagaaccttaccaagtcttgacatcctgcgacgcacatag |
| | | aaatatgtgtttccttcgggacgcagagacaggtggtgcatggttgtcgtcagctcgtgtcgtgagatgttgggttaagtcccgc |
| | | aacgagcgcaacccttatggtcagttactacgcaagaggactctggccagactgccgttgacaaaacggaggaaggtgggg |
| | | atgacgtcaaatcatcatgccctttatgacttgggctacacacgtactacaatggcgttaaacaaagagaagcaagaccgcgag |
| | | gtggagcaaaactcagaaacaacgtcccagttcggactgcaggctgcaactcgcctgcacgaagtcggaattgctagtaatcg |
| | | cagatcagcatgctgcggtgaatacgttcccgggccttgtacacaccgcccgtcacaccatgagagccggggggacccgaa |
| | | gtcggtagtctaaccgcaaggaggacgccgccgaaggtaaaactggtgattggggtgaagtcgtaacaaggtagccgtagg |
| | | agaacctgcggctggatcacctccttt |
| Faecalibacterium_ | SEQ ID | tataaagagtttgatcctggctcaggacgaacgctggcggcgcgcctaacacatgcaagtcgaacgagcgagagagagcttg |
| prausnitzii | NO: 79 | ctttctcaagcgagtggcgaacgggtgagtaacgcgtgaggaacctgcctcaaagagggggacaacagttggaaacgactg |
| | | ctaataccgcataagcccacgacccggcatcgggtagagggaaaaggagcaatccgctttgagatggcctcgcgtccgatta |
| | | gctagttggtgaggtaatggcccaccaaggcgacgatcggtagccggactgagaggttgaacggccacattgggactgaga |
| | | cacggcccagactcctacgggaggcagcagtggggaatattgcacaatgggggaaaccctgatgcagcgacgccgcgtgg |
| | | aggaagaaggtcttcggattgtaaactcctgttgttgaggaagataatgacggtactcaacaaggaagtgacggctaactacgt |
| | | gccagcagccgcggtaaaacgtaggtcacaagcgttgtccggaattattgggtgtaaagggagcgcaggcgggaagacaa |
| | | gttggaagtgaaatccatgggctcaacccatgaactgctttcaaaactgtttttcttgagtagtgcagaggtaggcggaattcccg |
| | | gtgtagcggtggaatgcgtagatatcgggaggaacaccagtggcgaaggcggcctactgggcaccaactgacgctgaggct |
| | | cgaaagtgtgggtagcaaacaggattagataccctggtagtccacactgtaaacgatgattactaggtgttggaggattgaccc |
| | | cttcagtgccgcagttaacacaataagtaatccacctggggagtacgaccgcaaggttgaaactcaaaggaattgacggggg |
| | | cccgcacaagcagtggagtatgtggtttaattcgacgcaacgcgaagaaccttaccaagtcttgacatcctgcgacgcacatag |
| | | aaatatgtgtttccttcgggacgcagagacaggtggtgcatggttgtcgtcagctcgtgtcgtgagatgttgggttaagtcccgc |
| | | aacgagcgcaacccttatggtcagttactacgcaagaggactctggccagactgccgttgacaaaacggaggaaggtgggg |
| | | atgacgtcaaatcatcatgccctttatgacttgggctacacacgtactacaatggcgttaaacaaagagaagcaagaccgcgag |
| | | gtggagcaaaactcagaaacaacgtcccagttcggactgcaggctgcaactcgcctgcacgaagtcggaattgctagtaatcg |
| | | cagatcagcatgctgcggtgaatacgttcccgggccttgtacacaccgcccgtcacaccatgagagccggggggacccgaa |
| | | gtcggtagtctaaccgcaaggaggacgccgccgaaggtaaaactggtgattggggtgaagtcgtaacaaggtagccgtagg |
| | | agaacctgcggctggatcacctccttt |
| Clostridium_ | SEQ ID | atttgagagtttgatcctggctcaggatgaacgaggcggcgtgcctaacacatgcaagtcgagcgattctcttcggagaagag |
| bartlettii | NO: 80 | cggcggacgggtgagtaacgcgtgggtaacctgccctgtacacacggataacataccgaaaggtatgctaatacgggataac |
| | | ataagaaattcgcatgtttttcttatcaaagctccggcggtacaggatggacccgcgtctgattagctagttggtgaggtaacggc |
| | | tcaccaaggcgacgatcagtagccgacctgagagggtgatcggccacattggaactgagacacggtccaaactcctacggg |
| | | aggcagcagtggggaatattgcacaatgggcgaaagcctgatgcagcaacgccgcgtgagcgatgaaggccttcgggtcgt |
| | | aaagctctgtcctcaaggaagataatgacggtacttgaggaggaagccccggctaactacgtgccagcagccgcggtaatac |
| | | gtagggggctagcgttatccggatttactgggcgtaaagggtgcgtaggcggtcttttaagtcaggagtgaaaggctacggct |
| | | caaccgtagtaagctcttgaaactggaggacttgagtgcaggagaggagagtggaattcctagtgtagcggtgaaatgcgtag |
| | | atattaggaggaacaccagtagcgaaggcggctctctggactgtaactgacgctgaggcacgaaagcgtggggagcaaaca |
| | | ggattagataccctggtagtccacgccgtaaacgatgagtactaggtgtcgggggttacccccctcggtgccgcagctaacgc |
| | | attaagtactccgcctggggagtacgctcgcaagagtgaaactcaaaggaattgacggggacccgcacaagtagcggagca |
| | | tgtggtttaattcgaagcaacgcgaagaaccttacctaagcttgacatccttttgaccgatgcctaatcgcatctttcccttcgggg |
| | | acagaagtgacaggtggtgcatggttgtcgtcagctcgtgtcgtgagatgttgggttaagtcccgcaacgagcgcaacccttgc |
| | | ctttagttgccatcattaagttgggcactctagagggactgccagggataacctggaggaaggtggggatgacgtcaaatcatc |
| | | atgccccttatgcttagggctacacacgtgctacaatgggtggtacagagggcagcgaagtcgtgaggccaagctaatccctt |
| | | aaagccattctcagttcggattgtaggctgaaactcgcctacatgaagctggagttactagtaatcgcagatcagaatgctgcgg |
| | | tgaatgcgttcccgggtcttgtacacaccgcccgtcacaccatgggagttgggggcgcccgaagccggctagctaaccttttg |
| | | gaagcggtcgtcgaaggtgaaaccaataactggggtgaagtcgtaacaaggtagccgtatcggaaggtgcggctggatcac |
| | | ctccttt |
| Clostridium_ | SEQ ID | atttgagagtttgatcctggctcaggatgaacgctggcggcgtgcctaacacatgcaagtcgagcgattctctttggagaagag |
| bartlettii | NO: 81 | cggcggacgggtgagtaacgcgtgggtaacctgccctgtacacarggataacataccgaaaggtatgctaatacgggataac |
| | | ataagaaattcgcatgtttttcttatcaaagctccggcggtacaggatggacccgcgtctgattagctagaggtgaggtaacggc |
| | | tcaccaaggcgacgatcagtagccgacctgagagggtgatcggccacattggaactgagacacggtccaaactcctacggg |
| | | aggcagcagtggggaatattgcacaatgggcgaaagcctgatgcagcaacgccgcgtgagcgatgaaggccttcgggtcgt |
| | | aaagctctgtcctcaaggaagataatgacggtacttgaggaggaagccccggctaactacgtgccagcagccgcggtaatac |
| | | gtagggggctagcgttatccggatttactgggcgtaaagggtgcgtaggcggtcttttaagtcaggagtgaaaggctacggct |
| | | caaccgtagtaagctcttgaaactgggggacttgagtgcaggagaggagagtggaattcctagtgtagcggtgaaatgcgtag |
| | | atattaggaggaacaccagtagcgaaggcggctctctggactgtaactgacgctgaggcacgaaagcgtggggagcaaaca |
| | | ggattagataccctggtagtccacgccgtaaacgatgagtactagctgtcggaggttacccccttcggtggcgcagctaacgc |
| | | attaagtactccgcctggggagtacgctcgcaagagtgaaactcaaaggaattgacggggacccgcacaagtagcggagca |
| | | tgtggtttaattcgaagcaacgcgaagaaccttacctaagcttgacatccttttgaccgatgcctaatcgcatctttcccttcgggg |
| | | acagaagtgacaggtggtgcatggttgtcgtcagctcgtgtcgtgagatgttgggttaagtcccgcaacgagcgcaacccttgc |
| | | ctttagttgccatcattaagttgggcactctagagggactgccagggataacctggaggaaggtggggatgacgtcaaatcatc |
| | | atgccccttatgcttagggctacacacgtgctacaatgggtggtacagagggcagcgaagtcgtgaggccaagctaatccctt |
| | | aaagccattctcagttcggattgtaggctgaaactcgcctacatgaagctggagttactagtaatcgcagatcagaatgctgcgg |
| | | tgaatgcgttcccgggtcttgtacacaccgcccgtcacaccatgggagttgggggcgcccgaagccggctagctaaccttttg |
| | | gaagcggtcgtcgaaggtgaaaccaataactggggtgaagtcgtaacaaggtagccgtatcggaaggtgcggctggatcac |
| | | ctccttt |
| Clostridium_ | SEQ ID | gaggcagcagtggggaatattgcacaatgggcgaaagcctgatgcagcaacgccgcgtgagcgatgaaggccttcgggtc |
| bartlettii | NO: 82 | gtaaagctctgtcctcaaggaagataatgacggtacttgaggaggaagccccggctaactacgtgccagcagccgcggtaat |
| | | acgtagggggctagcgttatccggatttactgggcgtaaagggtgcgtaggcggtcttttaagtcaggagtgaaaggctacgg |
| | | ctcaaccgtagtaagctcttgaaactggaggacttgagtgcaggagaggagagtggaattcctagtgtagcggtgaaatgcgt |
| | | agatattaggaggaacaccagtagcgaaggcggctctctggactgtaactgacgctgaggcacgaaagcgtggggagcaaa |
| | | caggattagataccctggtagtccacgccgtaaacgatgagtactagctgtcggaggttacccccttcggtggcgcagctaac |
| | | gcattaagtactccgcctggggagtacgctcgcaagagtgaaactcaaaggaattgacggggacccgcacaagtagcggag |
| | | catgtggtttaattcgaagcaacgcgaagaaccttacctaagcttgacatccttttgaccgatgcctaatcgcatctttcccttcgg |
| | | ggacagaagtgacaggtggtgcatggttgtcgtcagctcgtgtcgtgagatgagggttaagtcccgcaacgagcgcaaccct |
| | | tgcctttagttgccatcattaagttgggcactctagagggactgccagggataacctggaggaaggtggggatgacgtcaaatc |
| | | atcatgccccttatgcttagggctacacacgtgctacaatgggtggtacagagggcagcgaagtcgtgaggccaagctaatcc |
| | | cttaaagccattctcagttcggattgtaggctgaaactcgcctacatgaagctggagttactagtaatcgcagatcagaatgctgc |
| | | ggtgaatgcgttcccgggtcttgtacacaccgcccgtcacaccatgggagttgggggcgcccgaagccggctagctaaccttt |
| | | tggaagcggtcgtcgaaggtgaaaccaataactggggtgaagtcgtaacaaggtagccgtatcggaaggtgcggaggatc |
| | | acctccttt |
| Clostridium_ | SEQ ID | atttgagagtttgatcctggctcaggatgaacgctggcggcgtgcctaacacatgcaagtcgagcgatcttcttcggaagagag |
| bartlettii | NO: 83 | cggcggacgggtgagtaacgcgtgggtaacctgccctgtacacacggataacataccgaaaggtatgctaatacgggataac |
| | | ataagaaattcgcatgtttttcttatcaaagctccggcggtacaggatggacccgcgtctgattagctagttggtgaggtaacggc |
| | | tcaccaaggcgacgatcagtagccgacctgagagggtgatcggccacattggaactgagacacggtccaaactcctacggg |
| | | aggcagcagtggggaatattgcacaatgggcgaaagcctgatgcagcaacgccgcgtgagcgatgaaggccttcgggtcgt |
| | | aaagctctgtcctcaaggaagataatgacggtacttgaggaggaagccccggctaactacgtgccagcagccgcggtaatac |
| | | gtagggggctagcgttatccggatttactgggcgtaaagggtgcgtaggcggtcttttaagtcaggagtgaaaggctacggct |
| | | caaccgtagtaagctcttgaaactggaggacttgagtgcaggagaggagagtggaattcctagtgtagcggtgaaatgcgtag |
| | | atattaggaggaacaccagtagcgaaggcggctctctggactgtaactgacgctgaggcacgaaagcgtggggagcaaaca |
| | | ggattagataccctggtagtccacgccgtaaacgatgagtactagctgtcggaggttacccccttcggtggcgcagctaacgc |
| | | attaagtactccgcctggggagtacgctcgcaagagtgaaactcaaaggaattgacggggacccgcacaagtagcggagca |
| | | tgtggtttaattcgaagcaacgcgaagaaccttacctaagcttgacatccttttgaccgatgcctaatcgcatctttcccttcgggg |
| | | acagaagtgacaggtggtgcatggttgtcgtcagctcgtgtcgtgagatgttgggttaagtcccgcaacgagcgcaacccttgc |
| | | ctttagttgccatcattaagttgggcactctagagggactgccagggataacctggaggaaggtggggatgacgtcaaatcatc |
| | | atgccccttatgcttagggctacacacgtgctacaatgggtggtacagagggcagcgaagtcgtgaggccaagctaatccctt |
| | | aaagccattctcagttcggattgtaggctgaaactcgcctacatgaagctggagttactagtaatcgcagatcagaatgctgcgg |
| | | tgaatgcgttcccgggtcttgtacacaccgcccgtcacaccatgggagttgggggcgcccgaagccggctagctaaccttttg |
| | | gaagcggtcgtcgaaggtgaaaccaataactggggtgaagtcgtaacaaggtagccgtatcggaaggtgcggctggatcac |
| | | ctccttt |
| Clostridium_ | SEQ ID | atgcagtcgagcgatcttcttcggagagagcggcggacgggggagtaacgcgtgggtaacctgccctgtacacacggattac |
| bartlettii | NO: 84 | ataccgaaaggtatgctaatacgggataacataagaaattcgcatgtttttcttatcaaagctccggcggtacaggatggacccg |
| | | cgtctgattagctagttggtgaggtaacggctcaccaaggcgacgatcagtagccgacctgagagggtgatcggccacattg |
| | | gaactgagacacggtccaaactcctacgggaggcagcagtggggaatattgcacaatgggcgaaagcctgatgcagcaac |
| | | gccgcgtgagcgatgaaggccttcgggtcgtaaagctctgtcctcaaggaagataatgacggtacttgaggaggaagccccg |
| | | gctaactacgtgccagcagccgcggtaatacgtagggggctagcgttatccggatttactgggcgtaaagggtgcgtaggcg |
| | | gtcttttaagtcaggagtgaaaggctacggctcaaccgtagtaagctcttgaaactggaggacttgagtgcaggagaggagag |
| | | tggaattcctagtgtagcggtgaaatgcgtagatattaggaggaacaccagtagcgaaggcggctctctggactgtaactgac |
| | | gctgaggcacgaaagcgtggggagcaaacaggattaaataccctggtattccacgccgtaaacgatgagtactagctgtcgg |
| | | aggttaccccctttcggtggcgcagctaacgcataaatactcgcctggggagtacgctcgctagagtgaaattaacagaattga |
| | | ctggatcctgcccaattagcggaacatgtgcttgactcgtaccaacggaaaaaccttagcattcctgcttccttgacgtg |
| Clostridium_ | SEQ ID | atttgagagtttgatcctggctcaggatgaacgctggcggcgtgcctaacacatgcaagtcgagcgattctatcggagaagag |
| bartlettii | NO: 85 | cggcggacgggtgagtaacgcgtgggtaacctgccctgtacacacggataacataccgaaaggtatgctaatacgggataac |
| | | ataagaaattcgcatgtttttcttatcaaagctccggcggtacaggatggacccgcgtctgattagctagttggtgaggtaacggc |
| | | tcaccaaggcgacgatcagtagccgacctgagagggtgatcggccacattggaactgagacacggtccaaactcctacggg |
| | | aggcagcagtggggaatattgcacaatgggcgaaagcctgatgcagcaacgccgcgtgagcgatgaaggccttcgggtcgt |
| | | aaagctctgtcctcaaggaagataatgacggtacttgaggaggaagccccggctaactacgtgccagcagccgcggtaatac |
| | | gtagggggctagcgttatccggatttactgggcgtaaagggtgcgtaggcggtcttttaagtcaggagtgaaaggctacggct |
| | | caaccgtagtaagctcttgaaactggaggacttgagtgcaggagaggagagtggaattcctagtgtagcggtgaaatgcgtag |
| | | atattaggaggaacaccagtagcgaaggcggctctctggactgtaactgacgctgaggcacgaaagcgtggggagcaaaca |
| | | ggattagataccctggtagtccacgccgtaaacgatgagtactagctgtcggaggttacccccttcggtggcgcagctaacgc |
| | | attaagtactccgcctggggagtacgctcgcaagagtgaaactcaaaggaattgacggggacccgcacaagtagcggagca |
| | | tgtggtttaattcgaagcaacgcgaagaaccttacctaagcttgacatccttttgaccgatgcctaatcgcatctttcccttcgggg |
| | | acagaagtgacaggtggtgcatggttgtcgtcagctcgtgtcgtgagatgttgggttaagtcccgcaacgagcgcaacccttgc |
| | | ctttagttgccatcattaagttgggcactctagagggactgccagggataacctggaggaaggtggggatgacgtcaaatcatc |
| | | atgccccttatgcttagggctacacacgtgctacaatgggtggtacagagggcagcgaagtcgtgaggccaagctaatccctt |
| | | aaagccattctcagttcggattgtaggctgaaactcgcctacatgaagctggagttactagtaatcgcagatcagaatgctgcgg |
| | | tgaatgcgttcccgggtcttgtacacaccgcccgtcacaccatgggagttgggggcgcccgaagccggctagctaaccttttg |
| | | gaagcggtcgtcgaaggtgaaaccaataactggggtgaagtcgtaacaaggtagccgtatcggaaggtgcggctggatcac |
| | | ctccttt |
| Clostridium_ | SEQ ID | atggagagtttgatcctggctcaggatgaacgctggcggcgtgcctaatacatgcaagtcgaacgcgagcacttgtgctcgag |
| ramosum | NO: 86 | tggcgaacgggtgagtaatacataagtaacctgccctagacagggggataactattggaaacgatagctaagaccgcataggt |
| | | acggacactgcatggtgaccgtattaaaagtgcctcaaagcactggtagaggatggacttatggcgcattagctggttggcgg |
| | | ggtaacggcccaccaaggcgacgatgcgtagccgacctgagagggtgaccggccacactgggactgagacacggcccag |
| | | actcctacgggaggcagcagtagggaattttcggcaatgggggaaaccctgaccgagcaacgccgcgtgaaggaagaagg |
| | | ttttcggattgtaaacttctgttataaaggaagaacggcggctacaggaaatggtagccgagtgacggtactttattagaaagcc |
| | | acggctaactacgtgccagcagccgcggtaatacgtaggtggcaagcgttatccggaattattgggcgtaaagagggagcag |
| | | gcggcagcaagggtctgtggtgaaagcctgaagcttaacttcagtaagccatagaaaccaggcagctagagtgcaggagag |
| | | gatcgtggaattccatgtgtagcggtgaaatgcgtagatatatggaggaacaccagtggcgaaggcgacgatctggcctgca |
| | | actgacgctcagtcccgaaagcgtggggagcaaataggattagataccctagtagtccacgccgtaaacgatgagtactaagt |
| | | gttggatgtcaaagttcagtgctgcagttaacgcaataagtactccgcctgagtagtacgttcgcaagaatgaaactcaaagga |
| | | attgacgggggcccgcacaagcggtggagcatgtggtttaattcgaagcaacgcgaagaaccttaccaggtcttgacatactc |
| | | ataaaggctccagagatggagagatagctatatgagatacaggtggtgcatggttgtcgtcagctcgtgtcgtgagatgttggg |
| | | ttaagtcccgcaacgagcgcaacccttatcgttagttaccatcattaagttggggactctagcgagactgccagtgacaagctg |
| | | gaggaaggcggggatgacgtcaaatcatcatgccccttatgacctgggctacacacgtgctacaatggatggtgcagaggga |
| | | agcgaagccgcgaggtgaagcaaaacccataaaaccattctcagttcggattgtagtctgcaactcgactacatgaagttggaa |
| | | tcgctagtaatcgcgaatcagcatgtcgcggtgaatacgttctcgggccttgtacacaccgcccgtcacaccacgagagttgat |
| | | aacacccgaagccggtggcctaaccgcaaggaaggagctgtctaaggtgggattgatgattggggtgattgtcgtaacaaggt |
| | | atccctacgggaacgtggggatggatcacctccttt |
| Clostridium_ | SEQ ID | cagcagccgcggtaatacgtagggggcaagcgttatccggatttactgggtgtaaagggagcgtagacggcgatgcaagcc |
| scindens | NO: 87 | agatgtgaaagcccggggctcaaccccgggactgcatttggaactgcgtggctggagtgtcggagaggcaggcggaattcc |
| | | tagtgtagcggtgaaatgcgtagatattaggaggaacaccagtggcgaaggcggcctgctggacgatgactgacgttgaggc |
| | | tcgaaagcgtggggagcaaacaggattagataccctggtagtccacgccgtaaacgatgactactaggtgtcgggtggcaag |
| | | gccattcggtgccgcagcaaacgcaataagtagtccacctggggagtacgttcgcaagaatgaaactcaaaggaattgacgg |
| | | ggacccgcacaagcggtggagcatgtggtttaattcgaagcaacgcgaagaaccttacctgatcttgacatcccgatgccaaa |
| | | gcgcgtaacgcgctctttcttcggaacatcggtgacaggtggtgcatggttgtcgtcagctcgtgtcgtgagatgttgggttaagt |
| | | cccgcaacgagcgcaacccctatcttcagtagccagcattttggatgggcactctggagagactgccagggagaacctggag |
| | | gaaggtggggatgacgtcaaatcatcatgccccttatgaccagggctacacacgtgctacaatggcgtaaacaaagggaggc |
| | | gaacccgcgagggtgggcaaatcccaaaaataacgtctcagttcggattgtagtctgcaactcgactacatgaagctggaatc |
| | | gctagtaatcgcgaatcagaatgtcgcggtgaatacgttcccgggtcttgtacacaccgcccgtcacaccatgggagtcagtaa |
| | | cgcccgaagccggtgacccaacccgtaagggagggagccgtcgaaggtgggaccgataactggggtgaagtcgtaacaa |
| | | ggtagccgtatcggaaggtgcggctggatcacctccttt |
| Clostridium_ | SEQ ID | aacgagagtttgatcctggctcaggatgaacgctggcggcgtgcctaacacatgcaagtcgaacgaagcgcctggccccga |
| scindens | NO: 88 | cttcttcggaacgaggagccttgcgactgagtggcggacgggtgagtaacgcgtgggcaacctgccttgcactgggggataa |
| | | cagccagaaatggctgctaataccgcataagaccgaagcgccgcatggcgcggcggccaaagccccggcggtgcaagat |
| | | gggcccgcgtctgattaggtagttggcggggtaacggcccaccaagccgacgatcagtagccgacctgagagggtgaccg |
| | | gccacattgggactgagacacggcccagactcctacgggaggcagcagtggggaatattgcacaatgggggaaaccctgat |
| | | gcagcgacgccgcgtgaaggatgaagtatttcggtatgtaaacttctatcagcagggaagaagatgacggtacctgactaaga |
| | | agccccggctaactacgtgccagcagccgcggtaatacgtagggggcaagcgttatccggatttactgggtgtaaagggagc |
| | | gtagacggcgatgcaagccagatgtgaaagcccggggctcaaccccgggactgcatttggaactgcgtggctggagtgtcg |
| | | gagaggcaggcggaattcctagtgtagcggtgaaatgcgtagatattaggaggaacaccagtggcgaaggcggcctgctgg |
| | | acgatgactgacgttgaggctcgaaagcgtggggagcaaacaggattagataccctggtagtccacgccgtaaacgatgact |
| | | actaggtgtcgggtggcaaggccattcggtgccgcagcaaacgcaataagtagtccacctggggagtacgttcgcaagaatg |
| | | aaactcaaaggaattgacggggacccgcacaagcggtggagcatgtggtttaattcgaagcaacgcgaagaaccttacctga |
| | | tcttgacatcccgatgccaaagcgcgtaacgcgctctttcttcggaacatcggtgacaggtggtgcatggttgtcgtcagctcgt |
| | | gtcgtgagatgttgggttaagtcccgcaacgagcgcaacccctatcttcagtagccagcttttaggatgggcactctggagaga |
| | | ctgccagggagaacctggaggaaggtggggatgacgtcaaatcatcatgccccttatgaccagggctacacacgtgctacaa |
| | | tggcgtaaacaaagggaggcgaacccgcgagggtgggcaaatcccaaaaataacgtctcagttcggattgtagtctgcaact |
| | | cgactacatgaagttggaatcgctagtaatcgcgaatcagaatgtcgcggtgaatacgttcccgggtcttgtacacaccgcccg |
| | | tcacaccatgggagtcagtaacgcccgaagccggtgacccaacccgtaagggagggagccgtcgaaggtgggaccgataa |
| | | ctggggtgaagtcgtaacaaggtagccgtatcggaaggtgcggctggatcacctccttt |
| Anaerotruncus_ | SEQ ID | caaagagtttgatcctggctcaggacgaacgctggcggcgcgcctaacacatgcaagtcgaacggagcttacgttttgaagttt |
| colihominis | NO: 89 | tcggatggatgaatgtaagcttagtggcggacgggtgagtaacacgtgagcaacctgcctttcagagggggataacagccgg |
| | | aaacggctgctaataccgcatgatgttgcgggggcacatgcccctgcaaccaaaggagcaatccgctgaaagatgggctcgc |
| | | gtccgattagccagttggcggggtaacggcccaccaaagcgacgatcggtagccggactgagaggttgaacggccacattg |
| | | ggactgagacacggcccagactcctacgggaggcagcagtgggggatattgcacaatgggcgaaagcctgatgcagcgac |
| | | gccgcgtgagggaagacggtcttcggattgtaaacctctgtctttggggaagaaaatgacggtacccaaagaggaagctccg |
| | | gctaactacgtgccagcagccgcggtaatacgtagggagcaagcgttgtccggaattactgggtgtaaagggagcgtaggc |
| | | gggatggcaagtagaatgttaaatccatcggctcaaccggtggctgcgttctaaactgccgttcttgagtgaagtagaggcagg |
| | | cggaattcctagtgtagcggtgaaatgcgtagatattaggaggaacaccagtggcgaaggcggcctgctgggctttaactgac |
| | | gctgaggctcgaaagcgtggggagcaaacaggattagataccctggtagtccacgccgtaaacgatgattactaggtgtggg |
| | | gggactgaccccttccgtgccgcagttaacacaataagtaatccacctggggagtacggccgcaaggttgaaactcaaagga |
| | | attgacgggggcccgcacaagcagtggagtatgtggtttaattcgaagcaacgcgaagaaccttaccaggtcttgacatcgga |
| | | tgcatagcctagagataggtgaagcccttcggggcatccagacaggtggtgcatggttgtcgtcagctcgtgtcgtgagatgtt |
| | | gggttaagtcccgcaacgagcgcaacccttattattagttgctacgcaagagcactctaatgagactgccgttgacaaaacgga |
| | | ggaaggtggggatgacgtcaaatcatcatgccccttatgacctgggctacacacgtactacaatggcactaaaacagagggc |
| | | ggcgacaccgcgaggtgaagcgaatcccgaaaaagtgtctcagttcagattgcaggctgcaacccgcctgcatgaagtcgg |
| | | aattgctagtaatcgcggatcagcatgccgcggtgaatacgttcccgggccttgtacacaccgcccgtcacaccatgggagtc |
| | | ggtaacacccgaagccagtagcctaaccgcaaggggggcgctgtcgaaggtgggattgatgactggggtgaagtcgtaaca |
| | | aggtagccgtatcggaaggtgcggctggatcacctccttt |
| Anaerotruncus_ | SEQ ID | caaagagtttgatcctggctcaggacgaacgctggcggcgcgcctaacacatgcaagtcgaacggagcttacgttttgaagttt |
| colihominis | NO: 90 | tcggatggacgaatgtaagcttagtggcggacgggtgagtaacacgtgagcaacctgcctttcagagggggataacagccg |
| | | gaaacggctgctaataccgcatgatgttgcgggggcacatgcccctgcaaccaaaggagcaatccgctgaaagatgggctc |
| | | gcgtccgattagccagttggcggggtaacggcccaccaaagcgacgatcggtagccggactgagaggttgaacggccacat |
| | | tgggactgagacacggcccagactcctacgggaggcagcagtgggggatattgcacaatgggcgaaagcctgatgcagcg |
| | | acgccgcgtgagggaagacggtcttcggattgtaaacctctgtctttggggaagaaaatgacggtacccaaagaggaagctc |
| | | cggctaactacgtgccagcagccgcggtaatacgtagggagcaagcgttgtccggaattactgggtgtaaagggagcgtag |
| | | gcgggatggcaagtagaatgttaaatccatcggctcaaccggtggctgcgttctaaactgccgttcttgagtgaagtagaggca |
| | | ggcggaattcctagtgtagcggtgaaatgcgtagatattaggaggaacaccagtggcgaaggcggcctgctgggctttaactg |
| | | acgctgaggctcgaaagcgtggggagcaaacaggattagataccctggtagtccacgccgtaaacgatgattactaggtgtg |
| | | gggggactgaccccttccgtgccgcagttaacacaataagtaatccacctggggagtacggccgcaaggttgaaactcaaag |
| | | gaattgacgggggcccgcacaagcagtggagtatgtggtttaattcgaagcaacgcgaagaaccttaccaggtcttgacatcg |
| | | gatgcatagcctagagataggtgaagcccttcggggcatccagacaggtggtgcatggttgtcgtcagctcgtgtcgtgagat |
| | | gagggttaagtcccgcaacgagcgcaacccttattattagttgctacgcaagagcactctaatgagactgccgttgacaaaacg |
| | | gaggaaggtggggatgacgtcaaatcatcatgccccttatgacctgggctacacacgtactacaatggcactaaaacagagg |
| | | gcggcgacaccgcgaggtgaagcgaatcccgaaaaagtgtctcagttcagattgcaggctgcaacccgcctgcatgaagtc |
| | | ggaattgctagtaatcgcggatcagcatgccgcggtgaatacgttcccgggccttgtacacaccgcccgtcacaccatgggag |
| | | tcggtaacacccgaagccagtagcctaaccgcaaggggggcgctgtcgaaggtgggattgatgactggggtgaagtcgtaa |
| | | caaggtagccgtatcggaaggtgcggctggatcacctccttt |
| Anaerotruncus_ | SEQ ID | tcaaagagtttgatcctggctcaggacgaacgctggcggcgcgcctaacacatgcaagtcgaacggagcttacgttttgaagtt |
| colihominis | NO: 91 | ttcggatggatgaatgtaagcttagtggcggacgggtgagtaacacgtgagcaacctgcctttcagagggggataacagccg |
| | | gaaacggctgctaataccgcatgatgttgcgggggcacatgcccctgcaaccaaaggagcaatccgctgaaagatgggctc |
| | | gcgtccgattagccagttggcggggtaacggcccaccaaagcgacgatcggtagccggactgagaggttgaacggccacat |
| | | tgggactgagacacggcccagactcctacgggaggcagcagtgggggatattgcacaatgggcgaaagcctgatgcagcg |
| | | acgccgcgtgagggaagacggtcttcggattgtaaacctctgtctttggggaagaaaatgacggtacccaaagaggaagctc |
| | | cggctaactacgtgccagcagccgcggtaatacgtagggagcaagcgttgtccggaattactgggtgtaaagggagcgtag |
| | | gcgggatggcaagtagaatgttaaatccatcggctcaaccggtggctgcgttctaaactgccgttcttgagtgaagtagaggca |
| | | ggcggaattcctagtgtagcggtgaaatgcgtagatattaggaggaacaccagtggcgaaggcggcctgctgggctttaactg |
| | | acgctgaggctcgaaagcgtggggagcaaacaggattagataccctggtagtccacgccgtaacgatgattactaggtgtgg |
| | | ggggactgaccccttccgtgccgcagttaacacataagtaatccacctggggagtacggccgcaggtgaactcaaggattga |
| | | cggggcccgcacagcagtgagtatgtggtttatcgagcacgcgagactacagtctgacatcgatgcatagctagagatagtga |
| | | gccctcgggcatccagacaggtgggtgcatgggtgtcg |
| Anaerotruncus_ | SEQ ID | caaagagtttgatcctggctcaggacgaacgctggcggcgcgcctaacacatgcaagtcgaacggagcttacgttttgaagttt |
| colihominis | NO: 92 | tcggatggatgaatgtaagcttagtggcggacgggtgagtaacacgtgagcaacctgcctttcagagggggataacagccgg |
| | | aaacggctgctaataccgcatgatgttgcgggggcacatgcccctgcaaccaaaggagcaatccgctgaaagatgggctcgc |
| | | gtccgattagccagttggcggggtaacggcccaccaaagcgacgatcggtagccggactgagaggttgaacggccacattg |
| | | ggactgagacacggcccagactcctacgggaggcagcagtgggggatattgcacaatgggcgaaagcctgatgcagcgac |
| | | gccgcgtgagggaagacggtcttcggattgtaaacctctgtctttggggaagaaaatgacggtacccaaagaggaagctccg |
| | | gctaactacgtgccagcagccgcggtaatacgtagggagcaagcgttgtccggaattactgggtgtaaagggagcgtaggc |
| | | gggatggcaagtagaatgttaaatccatcggctcaaccggtggctgcgttctaaactgccgttcttgagtgaagtagaggcagg |
| | | cggaattcctagtgtagcggtgaaatgcgtagatattaggaggaacaccagtggcgaaggcggcctgctgggctttaactgac |
| | | gctgaggctcgaaagcgtggggagcaaacaggattagataccctggtagtccacgccgtaaacgatgattactaggtgtggg |
| | | gggactgaccccttccgtgccgcagttaacacaataagtaatccacctggggagtacggccgcaaggttgaaactcaaagga |
| | | attgacgggggcccgcacaagcagtggagtatgtggtttaattcgaagcaacgcgaagaaccttaccaggtcttgacatcgga |
| | | tgcatagcctagagataggtgaagcccttcggggcatccagacaggtggtgcatggttgtcgtcagctcgtgtcgtgagatgtt |
| | | gggttaagtcccgcaacgagcgcaacccttattattagttgctacgcaagagcactctaatgagactgccgttgacaaaacgga |
| | | ggaaggtggggatgacgtcaaatcatcatgccccttatgacctgggctacacacgtactacaatggcactaaaacagagggc |
| | | ggcgacaccgcgaggtgaagcgaatcccgaaaaagtgtctcagttcagattgcaggctgcaacccgcctgcatgaagtcgg |
| | | aattgctagtaatcgcggatcagcatgccgcggtgaatacgttcccgggccttgtacacaccgcccgtcacaccatgggagtc |
| | | ggtaacacccgaagccagtagcctaaccgcaaggggggcgctgtcgaaggtgggattgatgactggggtgaagtcgtaaca |
| | | aggtagccgtatcggaaggtgcggctggatcacctccttt |
| Clostridium_ | SEQ ID | atggagagtttgatcctggctcaggatgaacgctggcggcgtgcctaatacatgcaagtcgaacgcgagcagcaatgctcga |
| spiroforme | NO: 93 | gtggcgaacgggtgagtaatacataagtaacctgccctagacagggggataactgctggaaacggcagctaagaccgcata |
| | | ggtatggacactgcatggtgaccatattaaaagtgccaaggcactggtagaggatggacttatggcgcattagctggttggtga |
| | | ggtaacagctcaccaaggcgacgatgcgtagccgacctgagagggtgaccggccacactgggactgagacacggcccag |
| | | actcctacgggaggcagcagtagggaattttcggcaatggggggaaccctgaccgagcaacgccgcgtgaaggaagaagg |
| | | aattcgttctgtaaacttctgttataaaggaagaacggcggatatagggaatgatatccgagtgacggtactttatgagaaagcc |
| | | acggctaactacgtgccagcagccgcggtaatacgtaggtggcgagcgttatccggaattattgggcgtaaagagggagcag |
| | | gcggcggcagaggtctgtggtgaaagactgaagcttaacttcagtaagccatagaaaccgggctgctagagtgcaggagag |
| | | gatcgtggaattccatgtgtagcggtgaaatgcgtagatatatggaggaacaccagtggcgaaggcgacggtctggcctgtaa |
| | | ctgacgctcattcccgaaagcgtggggagcaaataggattagataccctagtagtccacgccgtaaacgatgagtactaagtgt |
| | | tgggagtcaaatttcagtgctgcagttaacgcaataagtactccgcctgagtagtacgttcgcaagaatgaaactcaaaggaatt |
| | | gacgggggcccgcacaagcggtggagcatgtggtttaattcgaagcaacgcgaagaaccttaccaggtcttgacatccggat |
| | | aaagacctcagagatgaggggatagatatatccgagacaggtggtgcatggttgtcgtcagctcgtgtcgtgagatgttgggtt |
| | | aagtcccgcaacgagcgcaacccttgtcgctagttaccatcattaagaggggactctagcgagactgccagtgacaagctgg |
| | | aggaaggcggggatgacgtcaaatcatcatgccccttatgacctggctacacacgtgctacaatgatggagcagagggaagc |
| | | gaagccgcgagtgagcaaacccataaaccattctcagtcggattgtagtctgcactcgactacatga |
| Clostridium_ | SEQ ID | atggagagtttgatcctggctcaggatgaacgctggcggcgtgcctaatacatgcaagtcgaacgcgagcagcaatgctcga |
| spiroforme | NO: 94 | gtggcgaacgggtgagtaatacataagtaacctgccctagacagggggataactgctggaaacggcagctaagaccgcata |
| | | ggtatggacactgcatggtgaccatattaaaagtgccaaggcactggtagaggatggacttatggcgcattagctggttggtga |
| | | ggtaacggctcaccaaggcgacgatgcgtagccgacctgagagggtgaccggccacactgggactgagacacggcccag |
| | | actcctacgggaggcagcagtagggaattttcggcaatggggggaaccctgaccgagcaacgccgcgtgaaggaagaagg |
| | | aattcgttctgtaaacttctgttataaaggaagaacggcggatatagggaatgatatccgagtgacggtactttatgagaaagcc |
| | | acggctaactacgtgccagcagccgcggtaatacgtaggtggcgagcgttatccggaattattgggcgtaaagagggagcag |
| | | gcggcggcagaggtctatggtgaaagactgaagcttaacttcagtaagccatagaaaccgggctgctagagtgcaggagag |
| | | gatcgtgaattccatgtgtagcggtgaaatgcgtagatatatggaggaacaccagtggcgaaggcgacggtctgcctgtaact |
| | | gacgctcattcccgaaagcgtggggagcaaataggattagataccctagtagtccacgccgtaacgatgagtactaagtgtgg |
| | | gagtcaaatttcagtgctgcagttaacgcaataagtacctccgccttgaatagtacgttcgcaaga |
| Clostridium_ | SEQ ID | atggagagtttgatcctggctcaggatgaacgctggcggcgtgcctaatacatgcaagtcgaacgcgagcagcaatgctcga |
| spiroforme | NO: 95 | gtggcgaacgggtgagtaatacataagtaacccgccctagacagggggataactgctggaaacggcagctaagaccgcata |
| | | ggtatggacactgcatggtgaccatattaaaagtgccaaggcactggtagaggatggacttatggcgcattagctggttggtga |
| | | ggtaacggctcaccaaggcgacgatgcgtagccgacctgagagggtgaccggccacactgggactgagacacggcccag |
| | | actcctacgggaggcagcagtagggaattttcggcaatggggggaaccctgaccgagcaacgccgcgtgaaggaagaagg |
| | | aattcgttctgtaaacttctgttataaaggaagaacggcggatatagggaatgatatccgagtgacggtactttatgagaaagcc |
| | | acggctaactacgtgccagcagccgcggtaatacgtaggtggcgagcgttatccggaattattgggcgtaaagagggagcag |
| | | gcggcggcagaggtctatggtgaaagactgaagcttaacttcagtaagccatagaaaccgggctgctagagtgcaggagag |
| | | gatcgtggaattccatgtgtagcggtgaaatgcgtagatatatggaggaacaccagtggcgaaggcgacggtctggcctgtaa |
| | | ctgacgctcattcccgaaagcgtggggagcaaataggattagataccctagtagtccacgccgtaaacgatgagtactaagtgt |
| | | tgggagtcaaatttcagtgctgcagttaacgcaataagtactccgcctgagtagtacgttcgcaagaatgaaactcaaaggaatt |
| | | gacgggggcccgcacaagcggtggagcatgtggtttaattcgaagcaacgcgaagaaccttaccaggtcttgacatccggat |
| | | aaagacctcagagatgaggggatagatatatccgagacaggtggtgcatggttgtcgtcagctcgtgtcgtgagatgttgggtt |
| | | aagtcccgcaacgagcgcaacccttgtcgctagttaccatcattaagttggggactctagcgagactgccagtgacaagctgg |
| | | aggaaggcggggatgacgtcaaatcatcatgccccttatgacctgcgctacacacgtgctacaatgtatggagcagagggaa |
| | | gcgaagccgcgaggtgaagcaaacccataaaccattctcagttcggattgtagtctgcaactcgactacatgagtgtatcgcta |
| | | gtatcgcgatcagcatgtcgcgtgaatacgttctcggccagtacacaccccgcccgtcaacaccattg |
| Anaerofustis_ | SEQ ID | ttttgagagtttgatcctggctcaggacgaacgctggcggcgtgcttaacacatgcaagtcgaacgagaaacttataaatgatcc |
| stercorihominis | NO: 96 | ttcgggtgaatctataagcggacagtggcgaacgggtgagtaacgcgtaggtaaccaacctcatgcagggggatagcccag |
| | | ggaaacttggattaaacccgcataagaccacagcaccgcatggtgcaggggtaaaaactccggtggcatgagacggacctg |
| | | cgtcttattaggtagttggtgaggtaacggctcaccaagccaacgatgagtagccgacctgagagggtgatcggccacattgg |
| | | gactgagacacggcccagactcctacgggaggcagcagtggggaatattgcgcaatgggggaaaccctgacgcagcaac |
| | | gccgcgtgagcgatgaaggttttcggatcgtaaagctctgtctttggggaagataatgacggtacccaaggaggaagctccgg |
| | | ctaactacgtgccagcagccgcggtaatacgtagggagcaagcgttgtccggattcactgggcgtaaagagcacgtaggcg |
| | | gttaattaagtcaggtgtgaaagttttcggctcaaccggaaaagtgcacttgaaactggataacttgagtatcggagaggtaagc |
| | | ggaattcctagtgtagcggtgaaatgcgtagagattaggaagaacaccggtggcgaaggcggcttactggacgataactgac |
| | | gctgaggtgcgaaagcgtggggagcgaacaggattagataccctggtagtccacgccgtaaacgatgaatactaggtgttgg |
| | | ggtaactcagtgccgcagttaacacattaagtattccgcctggggagtacgctcgcaagagtgaaactcaaaggaattgacgg |
| | | gggcccgcacaagcagcggagcatgtggtttaattcgaagcaacgcgaagaaccttaccaggtcttgacatcccttgaccgcc |
| | | taagagattaggctttccttcgggacaaggagacaggtggtgcatggttgtcgtcagctcgtgtcgtgagatgttgggttaagtc |
| | | ccgcaacgagcgcaacccttatgtttagttactaacattcagttgaggactctagacagactgcccttgaaagagggaggaagg |
| | | tggggacgacgtcaaatcatcatgccccttacgacctgggctacacacgtgctacaatggtctgtacagagggttgcgaagca |
| | | gtgatgctaagctaatctcaaaaagcagatctcagttcggattgcaggctgcaactcgcctgcatgaagtcggagttgctagtaa |
| | | tcgcgaatcagaatgtcgcggtgaatgcgttcccgggccttgtacacaccgcccgtcacaccacgagagttggtaacacccga |
| | | agccagtgagctaaccattaggaggcagctgtcgaaggtgggatcagtaattggggtgaagtcgtaacaaggtagccgtatc |
| | | ggaaggtgcggctggatcacctccttt |
| Anaerofustis_ | SEQ ID | ttttgagagtttgatcctggctcaggacgaacgctggcggcgtgcttaacacatgcaagtcgaacgagaaacttataaatgatcc |
| stercorihominis | NO: 97 | ttcgggtgaatctataagcggacagtggcgaacgggtgagtaacgcgtaggtaaccaacctcatgcagggggatagcccag |
| | | ggaaacttggattaaacccgcataagaccacagcaccgcatggtgcaggggtaaaaactccggtggcatgagacggacctg |
| | | cgtcttattaggtagttggtgaggtaacggctcaccaagccaacgatgagtagccgacctgagagggtgatcggccacattgg |
| | | gactgagacacggcccagactcctacgggaggcagcagtggggaatattgcgcaatgggggaaaccctgacgcagcaac |
| | | gccgcgtgagcgatgaaggttttcggatcgtaaagctctgtctttggggaagataatgacggtacccaaggaggaagctccgg |
| | | ctaactacgtgccagcagccgcggtaatacgtagggagcaagcgttgtccggattcactgggcgtaaagagcacgtaggcg |
| | | gttaattaagtcaggtgtgaaagttttcggctcaaccggaaaagtgcacttgaaactggataacttgagtatcggagaggtaagc |
| | | ggaattcctagtgtagcggtgaaatgcgtagagattaggaagaacaccggtggcgaaggcggcttactggacgataactgac |
| | | gctgaggtgcgaaagcgtggggagcgaacaggattagataccctggtagtccacgccgtaaacgatgaatactaggtgttgg |
| | | ggtaactcagtgccgcagttaacacattaagtattccgcctggggagtacgctcgcaagagtgaaactcaaaggaattgacgg |
| | | gggcccgcacaagcagcggagcatgtggtttaattcgaagcaacgcgaagaaccttaccaggtcttgacatcccttgaccgcc |
| | | taagagattaggctttccttcgggacaaggagacaggtggtgcatggttgtcgtcagctcgtgtcgtgagatgttgggttaagtc |
| | | ccgcaacgagcgcaacccttatgtttagttactaacattcagttgaggactctagacagactgcccttgaaagagggaggaagg |
| | | tggggacgacgtcaaatcatcatgccccttacgacctgggctacacacgtgctacaatggtctgtacagagggttgcgaagca |
| | | gtgatgctaagctaatctcaaaaagcagatctcagttcggattgcaggctgcaactcgcctgcatgaagtcggagttgctagtaa |
| | | tcgcgaatcagaatgtcgcggtgaatgcgttcccgggccttgtacacaccgcccgtcacaccacgagagttggtaacacccga |
| | | agccagtgagctaaccattaggaggcagctgtcgaaggtgggatcagtaattggggtgaagtcgtaacaaggtagccgtatc |
| | | ggaaggtgcggctggatcacctccttt |
| Clostridiales_ | SEQ ID | tttgagagtttgatcctggctcaggatgaacgctggcggcgtgcctaacacatgcaagtcgaacgaagcatcttataggaagttt |
| bacterium_ | NO: 98 | tcggatggaatatgggatgactgagtggcggacgggtgagtaacgcgtggataacctgcctcacactgggggataacagtta |
| 1_7_47FAA | | gaaatggctgctaataccgcataagcgcacagtaccgcatggtacggtgtgaaaaacccaggtggtgtgagatggatccgcg |
| | | tctgattagccagttggcggggtaacggcccaccaaagcgacgatcagtagccgacctgagagggtgaccggccacattgg |
| | | gactgagacacggcccaaactcctacgggaggcagcagtggggaatattgcacaatgggcgaaagcctgatgcagcgacg |
| | | ccgcgtgagtgaagaagtatctcggtatgtaaagctctatcagcagggaagaaaatgacggtacctgactaagaagccccgg |
| | | ctaactacgtgccagcagccgcggtaatacgtagggggcaagcgttatccggatttactgggtgtaaagggagcgtagacgg |
| | | cgatgcaagtctgaagtgaaagcccggggctcaaccccgggactgctttggaaactgtgtggctggagtgcaggagaggta |
| | | agtggaattcctagtgtagcggtgaaatgcgtagatattaggaggaacaccagtggcgaaggcggcttactggactgtaactg |
| | | acgttgaggctcgaaagcgtggggagcaaacaggattagataccctggtagtccacgccgtaaacgatgaatgctaggtgtc |
| | | ggggggcaaagcccctcggtgccgccgctaacgcaataagcattccacctggggagtacgttcgcaagaatgaaactcaaa |
| | | ggaattgacggggacccgcacaagcggtggagcatgtggtttaattcgaagcaacgcgaagaaccttaccaagtcttgacatc |
| | | cccctgaccggacagtaacgtgtcccttccttcgggacaggggagacaggtggtgcatggttgtcgtcagctcgtgtcgtgag |
| | | atgttgggttaagtcccgcaacgagcgcaacccttatccttagtagccagcatttcgggtgggcactctagggagactgccagg |
| | | gataacctggaggaaggtggggatgacgtcaaatcatcatgccccttatgatttgggctacacacgtgctacaatggcgtaaac |
| | | aaagggaagcaccctgcgaaggtgagcaaatcccaaaaataacgtcccagttcggactgtagtctgcaacccgactacacga |
| | | agctggaatcgctagtaatcgcggatcagaatgccgcggtgaatacgttcccgggtcttgtacacaccgcccgtcacaccatg |
| | | ggagtcagcaacgcccgaagtcagtgacccaaccgaaaggagggagctgccgaaggcggggcaggtaactggggtgaa |
| | | gtcgtaacaaggtagccgtatcggaaggtgcggctggatcacctccttt |
| Coprococcus_ | SEQ ID | aacgagagtttgatcctggctcaggatgaacgctggcggcgtgcttaacacatgcaagtcgaacgaagcactttaacctgattc |
| comes | NO: 99 | ttcggatgaaggtttttgtgactgagtggcggacgggtgagtaacgcgtgggtaacctgcctcatacagggggataacagttag |
| | | aaatgactgctaataccgcataagaccacagagccgcatggctcggtgggaaaaactccggtggtatgagatggacccgcgt |
| | | ctgattaggtagttggtggggtaacggcctaccaagccaacgatcagtagccgacctgagagggtgaccggccacattggga |
| | | ctgagacacggcccaaactcctacgggaggcagcagtggggaatattgcacaatgggggaaaccctgatgcagcgacgcc |
| | | gcgtgagcgaagaagtatttcggtatgtaaagctctatcagcagggaagaaaatgacggtacctgactaagaagcaccggcta |
| | | aatacgtgccagcagccgcggtaatacgtatggtgcaagcgttatccggatttactgggtgtaaagggagcgtagacggctgt |
| | | gtaagtctgaagtgaaagcccggggctcaaccccgggactgctttggaaactatgcagctagagtgtcggagaggtaagtgg |
| | | aattcccagtgtagcggtgaaatgcgtagatattgggaggaacaccagtggcgaaggcggcttactggacgatgactgacgtt |
| | | gaggctcgaaagcgtggggagcaaacaggattagataccctggtagtccacgccgtaaacgatgactactaggtgtcgggg |
| | | agcaaagctcttcggtgccgcagcaaacgcaataagtagtccacctggggagtacgttcgcaagaatgaaactcaaaggaatt |
| | | gacggggacccgcacaagcggtggagcatgtggtttaattcgaagcaacgcgaagaaccttacctgctcttgacatcccggt |
| | | gaccggcgtgtaatgacgccttttcttcggaacaccggtgacaggtggtgcatggttgtcgtcagctcgtgtcgtgagatgttgg |
| | | gttaagtcccgcaacgagcgcaacccttatcttcagtagccagcaattcggatgggcactctggagagactgccagggataac |
| | | ctggaggaaggtggggatgacgtcaaatcatcatgccccttatgagcagggctacacacgtgctacaatggcgtaaacaaag |
| | | ggaagcgagcctgcgagggtaagcaaatctcaaaaataacgtctcagttcggattgtagtctgcaactcgactacatgaagctg |
| | | gaatcgctagtaatcgcgaatcagcatgtcgcggtgaatacgttcccgggtcttgtacacaccgcccgtcacaccatgggagtt |
| | | ggtaacgcccgaagtcagtgacccaaccgtaaggagggagctgccgaaggtgggaccgataactggggtgaagtcgtaac |
| | | aaggtagccgtatcggaaggtgcggctggatcacctccttt |
| Roseburia_ | SEQ ID | atgagagtttgatcctggctcaggatgaacgctggcggcgtgcttaacacatgcaagtcgaacgaagcactttatttgatttcttc |
| intestinalis | NO: 100 | ggaatgaagattttgtgactgagtggcggacgggtgagtaacgcgtgggtaacctgcctcatacagggggataacagttgga |
| | | aacgactgctaataccgcataagcgcacagggtcgcatgacctggtgtgaaaaactccggtggtatgagatggacccgcgtct |
| | | gattagccagttggtggggtaacggcctaccaaagcgacgatcagtagccgacctgagagggtgaccggccacattgggac |
| | | tgagacacggcccaaactcctacgggaggcagcagtggggaatattgcacaatgggggaaaccctgatgcagcgacgccg |
| | | cgtgagcgaagaagtatttcggtatgtaaagctctatcagcagggaagaagaaatgacggtacctgactaagaagcaccggct |
| | | aaatacgtgccagcagccgcggtaatacgtatggtgcaagcgttatccggatttactgggtgtaaagggagcgcaggcggta |
| | | cggcaagtctgatgtgaaagcccggggctcaaccccggtactgcattggaaactgtcggactagagtgtcggaggggtaagt |
| | | ggaattcctagtgtagcggtgaaatgcgtagatattaggaggaacaccagtggcgaaggcggcttactggacgattactgacg |
| | | ctgaggctcgaaagcgtggggagcaaacaggattagataccctggtagtccacgccgtaaacgatgaatactaggtgtcggg |
| | | gagtattgctcttcggtgccgcagcaaacgcaataagtattccacctggggagtacgttcgcaagaatgaaactcaaaggaatt |
| | | gacggggacccgcacaagcggtggagcatgtggtttaattcgaagcaacgcgaagaaccttaccaagtcttgacatcccggt |
| | | gacagaacatgtaatgtgttttctcttcggagcatcggtgacaggtggtgcatggagtcgtcagctcgtgtcgtgagatgttggg |
| Blautia_ | SEQ ID | cagagagtttgatcctggctcaggatgaacgctggcggcgtgcttaacacatgcaagtcgaacgaagcgatagagaacgga |
| hydrogenotrophica | NO: 101 | gatttcggttgaagttttctattgactgagtggcggacgggtgagtaacgcgtgggtaacctgccctatacagggggataacagt |
| | | tagaaatgactgctaataccgcataagcgcacagcttcgcatgaagcggtgtgaaaaactgaggtggtataggatggacccgc |
| | | gttggattagctagttggtgaggtaacggcccaccaaggcgacgatccatagccggcctgagagggtgaacggccacattgg |
| | | gactgagacacggcccaaactcctacgggaggcagcagtggggaatattgcacaatgggggaaaccctgatgcagcgacg |
| | | ccgcgtgaaggaagaagtatctcggtatgtaaacttctatcagcagggaagaaagtgacggtacctgactaagaagccccgg |
| | | ctaattacgtgccagcagccgcggtaatacgtaaggggcaagcgttatccggatttactgggtgtaaagggagcgtagacggt |
| | | ttggcaagtctgatgtgaaaggcatgggctcaacctgtggactgcattggaaactgtcagacttgagtgccggagaggcaagc |
| | | ggaattcctagtgtagcggtgaaatgcgtagatattaggaggaacaccagtggcgaaggcggcctgctggacggtaactgac |
| | | gttgaggctcgaaagcgtggggagcaaacaggattagataccctggtagtccacgctgtaaacgatgaatactaggtgtcgg |
| | | gtggcaaagccattcggtgccgcagcaaacgcaataagtattccacctggggagtacgttcgcaagaatgaaactcaaagga |
| | | attgacggggacccgcacaagcggtggagcatgtggtttaattcgaagcaacgcgaagaaccttaccaaatcttgacatccct |
| | | ctgaccgggaagtaatgttcccttttcttcggaacagaggagacaggtggtgcatggttgtcgtcagctcgtgtcgtgagatgtt |
| | | gggttaagtcccgcaacgagcgcaacccttattcttagtagccagcaggtagagctgggcactctagggagactgccaggga |
| | | taacctggaggaaggtggggatgacgtcaaatcatcatgccccttatgatttgggctacacacgtgctacaatggcgtaaacaa |
| | | agggaagcgaaggggtgacctggagcaaatctcaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gtcagtaacgcccgaagtcagtgacccaaccgaaaggagggagctgccgaaggtgggactgataactggggtgaagtcgt |
| | | aacaaggtagccgtatcggaaggtgcggctggatcacctccttt |
| Holdemania_ | SEQ ID | acctaggcgatgatacgtagccgacctgagagggtgaccggccacattgggactgagacacggcccaaactcctacggga |
| filiformis | NO: 102 | ggcagcagtagggaattttcggcaatgggcgaaagcctgaccgagcaacgccgcgtgagtgaagaaggccttcgggttgta |
| | | aagctctgttgtgaaggaagaacggctcatacagggaatggtatgggagtgacggtactttaccagaaagccacggctaacta |
| | | cgtgccagcagccgcggtaatacgtaggtggcgagcgttatccggaattattgggcgtaaagggtgcgcaggcggtttgttaa |
| | | gtttaaggtgaaagcgtggggcttaaccccatatagccttagaaactgacagactagagtacaggagagggcaatggaattcc |
| | | atgtgtagcggtaaaatgcgtagatatatggaggaacaccagtggcgaaggcggttgcctggcctgtaactgacgctcatgca |
| | | cgaaagcgtggggagcaaataggattagataccctagtagtccacgccgtaaacgatgagaactaagtgttggggaaactca |
| | | gtgctgcagttaacgcaataagttctccgcctggggagtatgcacgcaagtgtgaaactcaaaggaattgacgggggcccgc |
| | | acaagcggtggagtatgtggtttaattcgacgcaacgcgaagaaccttaccaggtcttgacatcccctgcaaagacatagagat |
| | | atgttggaggttatcagggagacaggtggtgcatggttgtcgtcagctcgtgtcgtgagatgttgggttaagtcccgcaacgag |
| | | cgcaacccttgtctttagttgctaacattaagttgaggactctagagagactgccggtgacaaaccggaggaaggtggggatg |
| | | acgtcaaatcatcatgccccttatgacctgggctacacacgtactacaatggcggatacaacgagaagcaagacagtgatgtg |
| | | gagcaaaactcagaaagtccgtctcagttcggattgaagtctgcaacccgacttcatgaagccggaatcgctagtaatcgcgg |
| | | atcagcatgccgcggtgaatacgttctcgggccttgtacacaccgcccgtcaaaccatgagagttggcaatacccgaagccgg |
| | | tggcctaacctcgcaagaggagggagccgtcgaaggtagggctgatgattggggttaagtcgtaacaaggtatccctacggg |
| | | aacgtggggatggatcacctccttt |
| Clostridium_ | SEQ ID | tttgagagtttgatcctggctcaggatgaacgctggcggcgtgcctaacacatgcaagtcgaacgaagcattttagatgaagtttt |
| asparagiforme | NO: 103 | cggatggattctgagatgactgagtggcggacgggtgagtaacacgtggataacctgcctcacactgggggacaacagttag |
| | | aaatgactgctaataccgcataagcgcacagtaccgcatggtacggtgtgaaaaactccggtggtgtgagatggatccgcgtc |
| | | tgattagccagttggcggggtaacggcccaccaaagcgacgatcagtagccgacctgagagggtgaccggccacattggga |
| | | ctgagacacggcccaaactcctacgggaggcagcagtggggaatattgcacaatgggcgaaagcctgatgcagcgacgcc |
| | | gcgtgagtgaagaagtatttcggtatgtaaagctctatcagcagggaagataatgacggtacctgactaagaagccccggcta |
| | | actacgtgccagcagccgcggtaatacgtagggggcaagcgttatccggatttactgggtgtaaagggagcgtagacggcat |
| | | ggcaagtctgaagtgaaaacccagggctcaaccctgggactgctttggaaactgtcaagctagagtgcaggagaggtaagtg |
| | | gaattcctagtgtagcggtgaaatgcgtagatattaggaggaacaccagtggcgaaggcggcttactggactgtaactgacgtt |
| | | gaggctcgaaagcgtggggagcaaacaggattagataccctggtagtccacgccgtaaacgatgagtgctaggtgttgggg |
| | | ggcaaagcccttcggtgccgtcgcaaacgcaataagcactccacctggggagtacgttcgcaagaatgaaactcaaaggaat |
| | | tgacggggacccgcacaagcggtggagcatgtggtttaattcgaagcaacgcgaagaaccttaccaagtcttgacatccccct |
| | | gaccggcgtgtaacggcgcctttccttcgggacaggggagacaggtggtgcatggttgtcgtcagctcgtgtcgtgagatgtt |
| | | gggttaagtcccgcaacgagcgcaacccttatccttagtagccagcattaagatgggcactctagggagactgccagggaca |
| | | acctggaggaaggtggggatgacgtcaaatcatcatgccccttatgatttgggctacacacgtgctacaatggcgtaaacaaa |
| | | gggaagcgaccctgcgaaggtgagcaaatctcaaaaataacgtcccagttcggactgtagtctgcaacccgactacacgaag |
| | | ctggaatcgctagtaatcgcgaatcagaatgtcgcggtgaatacgttcccgggtcttgtacacaccgcccgtcacaccatggga |
| | | gtcagcaacgcccgaagtcagtgacccaaccgaaaggagggagctgccgaaggcggggcaggtaactggggtgaagtcg |
| | | taacaaggtagccgtatcggaaggtgcggctggatcacctccttt |
| Blautia_wexlerae | SEQ ID | tcagagagtttgatcctggctcaggatgaacgctggcggcgtgcttaacacatgcaagtcgaacgggaattactttattgaaact |
| | NO: 104 | tcggtcgatttaatttaattctagtggcggacgggtgagtaacgcgtgggtaacctgccttatacagggggataacagtcagaaa |
| | | tggctgctaataccgcataagcgcacagagctgcatggctcagtgtgaaaaactccggtggtataagatggacccgcgttgga |
| | | ttagctagttggtggggtaacggcccaccaaggcgacgatccatagccggcctgagagggtgaacggccacattgggactg |
| | | agacacggcccagactcctacgggaggcagcagtggggaatattgcacaatgggggaaaccctgatgcagcgacgccgc |
| | | gtgaaggaagaagtatctcggtatgtaaacttctatcagcagggaagatagtgacggtacctgactaagaagccccggctaac |
| | | tacgtgccagcagccgcggtaatacgtagggggcaagcgttatccggatttactgggtgtaaagggagcgtagacggtgtgg |
| | | caagtctgatgtgaaaggcatgggctcaacctgtggactgcattggaaactgtcatacttgagtgccggaggggtaagcggaa |
| | | ttcctagtgtagcggtgaaatgcgtagatattaggaggaacaccagtggcgaaggcggcttactggacggtaactgacgttga |
| | | ggctcgaaagcgtggggagcaaacaggattagataccctggtagtccacgccgtaaacgatgaatactaggtgtcgggtggc |
| | | agagccattcggtgccgtcgcaaacgcagtaagtattccacctggggagtacgttcgcaagaatgaaactcaaaggaattgac |
| | | ggggacccgcacaagcggtggagcatgtggtttaattcgaagcaacgcgaagaaccttaccaagtcttgacatccgcctgact |
| | | gatccttaaccggatctttccttcgggacaggcgagacaggtggtgcatggttgtcgtcagctcgtgtcgtgagatgttgggtta |
| | | agtcccgcaacgagcgcaacccctatcctcagtagccagcatttaaggtgggcactctggggagactgccagggataacctg |
| | | gaggaaggcggggatgacgtcaaatcatcatgccccttatgatttgggctacacacgtgctacaatggcgtaaacaaagggaa |
| | | gcgagattgtgagatggagcaaatcccaaaaataacgtcccagttcggactgtagtctgcaacccgactacacgaagctggaa |
| | | tcgctagtaatcgcggatcagaatgccgcggtgaatacgttcccgggtcttgtacacaccgcccgtcacaccatggggagtca |
| | | gtaacgcccgaagtcagtgacctaaccgaaagggaggagctgccgaaggcgggaccgatgactggggtgaagtcgtaaca |
| | | aggtagccgtatcggaaggtgcggctggatcacctccttt |
| Blautia_wexlerae | SEQ ID | cagagagtttgatcctggctcaggatgaacgctggcggcgtgcttaacacatgcaagtcgaacgggaattactttattgaaactt |
| | NO: 105 | cggtcgatttaatttaattctagtggcggacgggtgagtaacgcgtgggtaacctgccttatacagggggataacagtcagaaat |
| | | ggctgctaataccgcataagcgcacagagctgcatggctcagtgtgaaaaactccggtggtataagatggacccgcgttggat |
| | | tagctagttggtggggtaacggcccaccaaggcgacgatccatagccggcctgagagggtgaacggccacattgggactga |
| | | gacacggcccagactcctacgggaggcagcagtggggaatattgcacaatgggggaaaccctgatgcagcgacgccgcgt |
| | | gaaggaagaagtatctcggtatgtaaacttctatcagcagggaagatagtgacggtacctgactaagaagccccggctaacta |
| | | cgtgccagcagccgcggtaatacgtagggggcaagcgttatccggatttactgggtgtaaagggagcgtagacggtgtggca |
| | | agtctgatgtgaaaggcatgggctcaacctgtggactgcattggaaactgtcatacttgagtgccggaggggtaagcggaattc |
| | | ctagtgtagcggtgaaatgcgtagatattaggaggaacaccagtggcgaaggcggcttactggacggtaactgacgttgagg |
| | | ctcgaaagcgtggggagcaaacaggattagataccctggtagtccacgccgtaaacgatgaatactaggtgtcggggagcat |
| | | ggctcttcggtgccgtcgcaaacgcagtaagtattccacctggggagtacgttcgcaagaatgaaactcaaaggaattgacgg |
| | | ggacccgcacaagcggtggagcatgtggtttaattcgaagcaacgcgaagaaccttaccaagtcttgacatccgcctgaccga |
| | | tccttaaccggatctttccttcgggacaggcgagacaggtggtgcatggttgtcgtcagctcgtgtcgtgagatgttgggttaagt |
| | | cccgcaacgagcgcaacccctatcctcagtagccagcatttaaggtgggcactctggggagactgccagggataacctggag |
| | | gaaggcggggatgacgtcaaatcatcatgccccttatgatttgggctacacacgtgctacaatggcgtaaacaaagggaagcg |
| | | agattgtgagatggagcaaatcccaaaaataacgtcccagttcggactgtagtctgcaacccgactacacgaagctggaatcg |
| | | ctagtaatcgcggatcagaatgccgcggtgaatacgttcccgggtcttgtacacaccgcccgtcacaccatgggagtcagtaa |
| | | cgcccgaagtcagtgacctaactgcaaagaaggagctgccgaaggcgggaccgatgactggggtgaagtcgtaacaaggt |
| | | agccgtatcggaaggtgcggctggatcacctccttt |
| Blautia_wexlerae | SEQ ID | cagagagtttgatcctggctcaggatgaacgctggcggcgtgcttaacacatgcaagtcgaacgggaattactttattgaaactt |
| | NO: 106 | cggtcgatttaatttaattctagtggcggacgggtgagtaacgcgtgggtaacctgccttatacagggggataacagtcagaaat |
| | | ggctgctaataccgcataagcgcacagagctgcatggctcagtgtgaaaaactccggtggtataagatggacccgcgttggat |
| | | tagctggttggtggggtaacggcccaccaaggcgacgatccatagccggcctgagagggtgaacggccacattgggactga |
| | | gacacggcccagactcctacgggaggcagcagtggggaatattgcacaatgggggaaaccctgatgcagcgacgccgcgt |
| | | gaaggaagaagtatctcggtatgtaaacttctatcagcagggaagatagtgacggtacctgactaagaagccccggctaacta |
| | | cgtgccagcagccgcggtaatacgtagggggcaagcgttatccggatttactgggtgtaaagggagcgtagacggtgtggca |
| | | agtctgatgtgaaaggcatgggctcaacctgtggactgcattggaaactgtcatacttgagtgccggaggggtaagcggaattc |
| | | ctagtgtagcggtgaaatgcgtagatattaggaggaacaccagtggcgaaggcggcttactggacggtaactgacgttgagg |
| | | ctcgaaagcgtggggagcaaacaggattagataccctggtagtccacgccgtaaacgatgaatactaggtgtcgggtagcat |
| | | agctcttcggtgccgtcgcaaacgcagtaagtattccacctggggagtacgttcgcaagaatgaaactcaaaggaattgacgg |
| | | ggacccgcacaagcggtggagcatgtggtttaattcgaagcaacgcgaagaaccttaccaagtcttgacatccctctgaccga |
| | | aacttaatcggatctttccttcgggacagacgagacaggtggtgcatggttgtcgtcagctcgtgtcgtgagatgttgggttaagt |
| | | cccgcaacgagcgcaacccctatcctcagtagccagcatttaaggtgggcactctggggagactgccagggataacctggag |
| | | gaaggcggggatgacgtcaaatcatcatgccccttatgatttgggctacacacgtgctacaatggcgtaaacaaagggaagcg |
| | | agattgtgagatggagcaaatcccaaaaataacgtcccagttcggactgtagtctgcaacccgactacacgaagctggaatcg |
| | | ctagtaatcgcggatcagaatgccgcggtgaatacgttcccgggtcttgtacacaccgcccgtcacaccatgggagtcagtaa |
| | | cgcccgaagtcagtgacctaactgcaaagaaggagctgccgaaggcgggaccgatgactggggtgaagtcgtaacaaggt |
| | | agccgtatcggaaggtgcggctggatcacctccttt |
| Blautia_wexlerae | SEQ ID | tcagagagtttgatcctggctcaggatgaacgctggcggcgtgcttaacacatgcaagtcgaacgggaaatatttcattgagac |
| | NO: 107 | ttcggtggatttgatctatttctagtggcggacgggtgagtaacgcgtgggtaacctgccttatacagggggataacagtcagaa |
| | | atggctgctaataccgcataagcgcacagagctgcatggctcagtgtgaaaaactccggtggtataagatggacccgcgttgg |
| | | attagctggttggtggggtaacggcccaccaaggcgacgatccatagccggcctgagagggtgaacggccacattgggact |
| | | gagacacggcccagactcctacgggaggcagcagtggggaatattgcacaatgggggaaaccctgatgcagcgacgccg |
| | | cgtgaaggaagaagtatctcggtatgtaaacttctatcagcagggaagatagtgacggtacctgactaagaagccccggctaa |
| | | ctacgtgccagcagccgcggtaatacgtagggggcaagcgttatccggatttactgggtgtaaagggagcgtagacggtgtg |
| | | gcaagtctgatgtgaaaggcatgggctcaacctgtggactgcattggaaactgtcatacttgagtgccggaggggtaagcgga |
| | | attcctagtgtagcggtgaaatgcgtagatattaggaggaacaccagtggcgaaggcggcttactggacggtaactgacgttg |
| | | aggctcgaaagcgtggggagcaaacaggattagataccctggtagtccacgccgtaaacgatgaatactaggtgtcgggga |
| | | gcatggctcttcggtgccgtcgcaaacgcagtaagtattccacctggggagtacgttcgcaagaatgaaactcaaaggaattg |
| | | acggggacccgcacaagcggtggagcatgtggtttaattcgaagcaacgcgaagaaccttaccaagtcttgacatccgcctg |
| | | accgatccttaaccggatctttccttcgggacaggcgagacaggtggtgcatggttgtcgtcagctcgtgtcgtgagatgttggg |
| | | ttaagtcccgcaacgagcgcaacccctatcctcagtagccagcatttaaggtgggcactctggggagactgccagggataacc |
| | | tggaggaaggcggggatgacgtcaaatcatcatgccccttatgatttgggctacacacgtgctacaatggcgtaaacaaaggg |
| | | aagcgagattgtgagatggagcaaatcccaaaaataacgtcccagttcggactgtagtctgcaacccgactacacgaagctgg |
| | | aa |
| Blautia_ | SEQ ID | cagagagtttgatcctggctcaggatgaacgctggcggcgtgcttaacacatgcaagtcgaacgggaattactttattgaaactt |
| wexlerae. | NO: 108 | cggtcgatttaatttaattctagtggcggacgggtgagtaacgcgtgggtaacctgccttatacagggggataacagtcagaaat |
| | | ggctgctaataccgcataagcgcacagagctgcatggctcagtgtgaaaaactccggtggtataagatggacccgcgttggat |
| | | tagctagttggtggggtaacggcccaccaaggcgacgatccatagccggcctgagagggtgaacggccacattgggactga |
| | | gacacggcccagactcctacgggaggcagcagtggggaatattgcacaatgggggaaaccctgatgcagcgacgccgcgt |
| | | gaaggaagaagtatctcggtatgtaaacttctatcagcagggaagatagtgacggtacctgactaagaagccccggctaacta |
| | | cgtgccagcagccgcggtaatacgtagggggcaagcgttatccggatttactgggtgtaaagggagcgtagacggtgtggca |
| | | agtctgatgtgaaaggcatgggctcaacctgtggactgcattggaaactgtcatacttgagtgccggaggggtaagcggaattc |
| | | ctagtgtagcggtgaaatgcgtagatattaggaggaacaccagtggcgaaggcggcttactggacggtaactgacgttgttgg |
| | | ctcgaaagcgtggggagcaaacaggattagataccctggtagtccacgccgtaaacgatgaatactaggtgtcggggagcat |
| | | ggctcttcggtgccgtcgcaaacgcagtaagtattccacctggggagtacgttcgcaagaatgaaactcaaaggaattgacgg |
| | | ggacccgcacaagcggtggagcatgtggtttaattcgaagcaacgcgaagaaccttaccaagtcttgacatccgcctgaccga |
| | | tccttaaccggatctttccttcgggacaggcgagacaggtggtgcatggttgtcgtcagctcgtgtcgtgagatgttgggttaagt |
| | | cccgcaacgagcgcaacccctatcctcagtagccagcatttaaggtgggcactctggggagactgccagggataacctggag |
| | | gaaggcggggatgacgtcaaatcatcatgccccttatgatttgggctacacacgtgctacaatggcgtaaacaaagggaagcg |
| | | agattgtgagatggagcaaatcccaaaaataacgtcccagttcggactgtagtctgcaacccgactacacgaagctggaatcg |
| | | ctagtaatcgcggatcagaatgccgcggtgaatacgttcccgggtcttgtacacaccgcccgtcacaccatgggagtcagtaa |
| | | cgcccgaagtcagtgacctaactgcaaagaaggagctgccgaaggcgggaccgatgactggggtgaagtcgtaacaaggt |
| | | agccgtatcggaaggtgcggctggatcacctccttt |
| Blautia_wexlerae | SEQ ID | tcagagagtttgatcctggctcaggatgaacgctggcggcgtgcttaacacatgcaagtcgaacgggaattactttattgaaact |
| | NO: 109 | tcggtcgatttaatttaattctagtggcggacgggtgagtaacgcgtgggtaacctgccttatacagggggataacagtcagaaa |
| | | tggctgctaataccgcataagcgcacagagagcatggctcagtgtgaaaaactccggtggtataagatggacccgcgttgga |
| | | ttagcttgttggtgaggtaacggcccaccaaggcgacgatccatagccggcctgagagggtgaacggccacattgggactga |
| | | gacacggcccagactcctacgggaggcagcagtggggaatattgcacaatgggggaaaccctgatgcagcgacgccgcgt |
| | | gaaggaagaagtatctcggtatgtaaacttctatcagcagggaagatagtgacggtacctgactaagaagccccggctaacta |
| | | cgtgccagcagccgcggtaatacgtagggggcaagcgttatccggatttactgggtgtaaagggagcgtagacggtgtggca |
| | | agtctgatgtgaaaggcatgggctcaacctgtggactgcattggaaactgtcatacttgagtgccggaggggtaagcggaattc |
| | | ctagtgtagcggtgaaatgcgtagatattaggaggaacaccagtggcgaaggcggcttactggacggtaactgacgttgagg |
| | | ctcgaaagcgtggggagcaaacaggattagataccctggtagtccacgccgtaaacgatgaatactaggtgtcggggagcat |
| | | ggctcttcggtgccgtcgcaaacgcagtaagtattccacctggggagtacgttcgcaagaatgaaactcaaaggaattgacgg |
| | | ggacccgcacaagcggtggagcatgtggtttaattcgaagcaacgcgaagaaccttaccaagtcttgacatccctctgaccgg |
| | | aacttaatcgttcctttccttcgggacagaggagacaggtggtgcatggttgtcgtcagctcgtgtcgtgagatgttgggttaagt |
| | | cccgcaacgagcgcaacccctatcctcagtagccagcatttaaggtgggcactctggggagactgccagggataacctggag |
| | | gaaggcggggatgacgtcaaatcatcatgccccttatgatttgggctacacacgtgctacaatggcgtaaacaaagggaagcg |
| | | agattgtgagatggagcaaatcccaaaaataacgtcccagttcggactgtagtctgcaacccgactacacgaagctggaatcg |
| | | ctagtaatcgcggatcagaatgccgcggtgaatacgttcccgggtcttgtacacaccgcccgtcacaccatgggagtcagtaa |
| | | cgcccgaagtcagtgacctaactgcaaagaaggagctgccgaaggcgggaccgatgactggggtgaagtcgtaacaaggt |
| | | agccgtatcggaaggtgcggctggatcacctccttt |
| Blautia_wexlerae | SEQ ID | tcagagagtttgatcctggctcaggatgaacgctggcggcgtgcttaacacatgcaagtcgaacgggaaatatttcattgagac |
| | NO: 110 | ttcggtggatttgatctatttctagtggcggacgggtgagtaacgcgtgggtaacctgccttatacagggggataacagtcagaa |
| | | atggctgctaataccgcataagcgcacagagctgcatggctcagtgtgaaaaactccggtggtataagatggacccgcgttgg |
| | | attagctggttggtggggtaacggcccaccaaggcgacgatccatagccggcctgagagggtgaacggccacattgggact |
| | | gagacacggcccagactcctacgggaggcagcagtggggaatattgcacaatgggggaaaccctgatgcagcgacgccg |
| | | cgtgaaggaagaagtatctcggtatgtaaacttctatcagcagggaagatagtgacggtacctgactaagaagccccggctaa |
| | | ctacgtgccagcagccgcggtaatacgtagggggcaagcgttatccggatttactgggtgtaaagggagcgtagacggtgtg |
| | | gcaagtctgatgtgaaaggcatgggctcaacctgtggactgcattggaaactgtcatacttgagtgccggaggggtaagcgga |
| | | attcctagtgtagcggtgaaatgcgtagatattaggaggaacaccagtggcgaaggcggcttactggacggtaactgacgttg |
| | | aggctcgaaagcgtggggagcaaacaggattagataccctggtagtccacgccgtaaacgatgaatactaggtgtcgggtgg |
| | | cagagccattcggtgccgtcgcaaacgcagtaagtattccacctggggagtacgttcgcaagaatgaaactcaaaggaattga |
| | | cggggacccgcacaagcggtggagcatgtggtttaattcgaagcaacgcgaagaaccttaccaagtcttgacatccctctgac |
| | | cggaacttaatcgttcctttccttcgggacagaggagacaggtggtgcatggttgtcgtcagctcgtgtcgtgagatgttgggtta |
| | | agtcccgcaacgagcgcaacccctatcctcagtagccagcatttaaggtgggcactctggggagactgccagggataacctg |
| | | gaggaaggcggggatgacgtcaaatcatcatgccccttatgatttgggctacacacgtgctacaatggcgtaaacaaagggaa |
| | | gcgagattgtgagatggagcaaatcccaaaaataacgtcccagttcggactgtagtctgcaacccgactacacgaagctggaa |
| | | tcgctagtaatcgcggatcagaatgccgcggtgaatacgttcccgggtcttgtacacaccgcccgtcacaccatgggagtcag |
| | | taacgcccgaagtcagtgacctaactgcaaagaaggagctgccgaaggcgggaccgatgactggggtgaagtcgtaacaa |
| | | ggtagccgtatcggaaggtgcggctggatcacctccttt |
| Dialister_ | SEQ ID | atggagagtttgatcctggctcaggacgaacgctggcggcgtgcttaacacatgcaagtcgaacgaaaagacggaaagagct |
| invisus | NO: 111 | tgctcttttcagaattgagtggcaaacgggtgagtaacacgtaaacaacctgccttcaggatggggacaacagacggaaacga |
| | | ctgctaataccgaataagttccaagagccgcatggcccatggaagaaaaggtggcctctacctgtaagctatcgcctgaagag |
| | | gggtttgcgtctgattagaggttggaggggtaacggcccaccaaggcgacgatcagtagccggtctgagaggatgaacggc |
| | | cacactggaactgagacacggtccagactcctacgggaggcagcagtggggaatcttccgcaatgggcgaaagcctgacg |
| | | gagcaacgccgcgtgagtgatgacggccttcgggttgtaaaactctgtgatccgggacgaaaaggcagagtgcgaagaaca |
| | | aactgcattgacggtaccggaaaagcaagccacggctaactacgtgccagcagccgcggtaatacgtaggtggcaagcgtt |
| | | gtccggaattattgggcgtaaagcgcgcgcaggcggcttcccaagtccctcttaaaagtgcggggcttaaccccgtgatggga |
| | | aggaaactgggaagctggagtatcggagaggaaagtggaattcctagtgtagcggtgaaatgcgtagagattaggaagaac |
| | | accggtggcgaaggcgactttctggacgaaaactgacgctgaggcgcgaaagcgtggggagcaaacaggattagataccct |
| | | ggtagtccacgccgtaaacgatggatactaggtgtaggaggtatcgacccctcctgtgccggagttaacgcaataagtatccc |
| | | gcctgggaagtacgatcgcaagattaaaactcaaaggaattgacggggcccgcacaagcggtggagtatgtggtttaattcga |
| | | cgcaacgcgaagaaccttaccaggtcttgacattgatcgcgatctgcagaaatgcggagttcttcttcggaagacgagaaaac |
| | | aggtggtgcacggctgtcgtcagctcgtgtcgtgagatgttgggttaagtcccgcaacgagcgcaacccctatcatttgttacca |
| | | gcacgtaaaggtggggactcaaatgagaccgccgcggacaacgcggaggaaggcggggacgacgtcaagtcatcatgcc |
| | | ccttatgacctgggctacacacgtactacaatgggtgtcaacaaagagaagcgaacccgcgaggaagagcaaacctcaaaa |
| | | acacacccccagttcagatcgcaggctgcaacccgcctgcgtgaagtaggaatcgctagtaatcgcgggtcagcataccgcg |
| | | gtgaatacgttcccgggccttgtacacaccgcccgtcacactatgagagtcagaaacacccgaagccggtgaggtaaccgca |
| | | aggagccagccgtcgaaggcggagctgatgattggagtgaagtcgtaacaaggtagccgtatcggaaggtgcggctggatc |
| | | acctccttt |
| Dialister_ | SEQ ID | atggagagtttgatcctggctcaggacgaacgctggcggcgtgcttaacacatgcaagtcgaacgaaaagagggaaagagc |
| invisus | NO: 112 | ttgctctttccggaattgagtggcaaacgggtgagtaacacgtaaacaacctgccttcaggatggggacaacagacggaaacg |
| | | actgctaataccgaataagttccaagagccgcatggcccatggaagaaaaggtggcctctacctgtaagctatcgcctgaaga |
| | | ggggtttgcgtctgattagctggttggaggggtaacggcccaccaaggcgacgatcagtagccggtctgagaggatgaacg |
| | | gccacactggaactgagacacggtccagactcctacgggaggcagcagtggggaatcttccgcaatgggcgaaagcctga |
| | | cggagcaacgccgcgtgagtgatgacggccttcgggttgtaaaactctgtgatccgggacgaaaaggcagagtgcgaagaa |
| | | caaactgcattgacggtaccggaaaagcaagccacggctaactacgtgccagcagccgcggtaatacgtaggtggcaagcg |
| | | ttgtccggaattattgggcgtaaagcgcgcgcaggcggcttcccaagtccctcttaaaagtgcggggcttaaccccgtgatgg |
| | | gaaggaaactgggaagctggagtatcggagaggaaagtggaattcctagtgtagcggtgaaatgcgtagagattaggaaga |
| | | acaccggtggcgaaggcgactttctggacgaaaactgacgctgaggcgcgaaagcgtggggagcaaacaggattagatac |
| | | cctggtagtccacgccgtaaacgatggatactaggtgtaggaggtatcgacccctcctgtgccggagttaacgcaataagtatc |
| | | ccgcctgggaagtacgatcgcaagattaaaactcaaaggaattgacgggggcccgcacaagcggtggagtatgtggtttaatt |
| | | cgacgcaacgcgaagaaccttaccaggtcttgacattgatcgcgatctgcagaaatgcggagttcttcttcggaagacgagaa |
| | | aacaggtggtgcacggctgtcgtcagctcgtgtcgtgagatgttgggttaagtcccgcaacgagcgcaacccctatcatttgtta |
| | | ccagcacgtaaaggtggggactcaaatgagaccgccgcggacaacgcggaggaaggcggggacgacgtcaagtcatcat |
| | | gccccttatgacctgggctacacacgtactacaatgggtgtcaacaaagagaagcgaacccgcgaggaagagcaaacctca |
| | | aaaacacacccccagttcagatcgcaggctgcaacccgcctgcgtgaagtaggaatcgctagtaatcgcgggtcagcatacc |
| | | gcggtgaatacgttcccgggccttgtacacaccgcccgtcacactatgagagtcagaaacacccgaagccggtgaggtaacc |
| | | gcaaggagccagccgtcgaaggcggagctgatgattggagtgaagtcgtaacaaggtagccgtatcggaaggtgcggctg |
| | | gatcacctccttt |
| Dialister_ | SEQ ID | atggagagtttgatcctggctcaggacgaacgctggcggcgtgcttaacacatgcaagtcgaacgaaaagacggaaagagct |
| invisus | NO: 113 | tgctcttttcagaattgagtggcaaacgggtgagtaacacgtaaacaacctgccttcaggatggggacaacagacggaaacga |
| | | ctgctaatgccgaataagttccaagagccgcatggcccatggaagaaaaggtggcctctacctgtaagctatcgcctgaagag |
| | | gggtttgcgtctgattagctggttggaggggtaacggcccaccaaggcgacgatcagtagccggtctgagaggatgaacggc |
| | | cacactggaactgagacacggtccagactcctacgggaggcagcagtggggaatcttccgcaatgggcgaaagcctgacg |
| | | gagcaacgccgcgtgagtgatgacggccttcgggttgtaaaactctgtgatccgggacgaaaaggcagagtgcgaagaaca |
| | | aactgcattgacggtaccggaaaagcaagccacggctaactacgtgccagcagccgcggtaatacgtaggtggcaagcgtt |
| | | gtccggaattattgggcgtaaagcgcgcgcaggcggcttcccaagtccctcttaaaagtgcggggcttaaccccgtgatggga |
| | | aggaaactgggaagctggagtatcggagaggaaagtggaattcctagtgtagcggtgaaatgcgtagagattaggaagaac |
| | | accggtggcgaaggcgactttctggacgaaaactgacgctgaggcgcgaaagcgtggggagcaaacaggattagataccct |
| | | ggtagtccacgccgtaaacgatggatactaggtgtaggaggtatcgacccctcctgtgccggagttaacgcaataagtatccc |
| | | gcctgggaagtacgatcgcaagattaaaactcaaaggaattgacgggggcccgcacaagcggtggagtatgtggtttaattcg |
| | | acgcaacgcgaagaaccttaccaggtcttgacattgatcgcgatctgcagaaatgcggagttcttcttcggaagacgagaaaa |
| | | caggtggtgcacggctgtcgtcagctcgtgtcgtgagatgttgggttaagtcccgcaacgagcgcaacccctatcatttgttacc |
| | | agcacgtaaaggtggggactcaaatgagaccgccgcggacaacgcggaggaaggcggggacgacgtcaagtcatcatgc |
| | | cccttatgacctgggctacacacgtactacaatgggtgtcaacaaagagaagcgaacccgcgaggaagagcaaacctcaaa |
| | | aacacacccccagttcagatcgcaggctgcaacccgcctgcgtgaagtaggaatcgctagtaatcgcgggtcagcataccgc |
| | | ggtgaatacgttcccgggccttgtacacaccgcccgtcacactatgagagtcagaaacacccgaagccggtgaggtaaccgc |
| | | aaggagccagccgtcgaaggcggagctgatgattggagtgaagtcgtaacaaggtagccgtatcggaaggtgcggctggat |
| | | cacctccttt |
| Dorea_ | SEQ ID | acgagagtttgatcctggctcaggatgaacgctggcggcgtgcttaacacatgcaagtcgagcgaagcacataagttcgattct |
| formicigenerans | NO: 114 | tcggatgaagacttttgtgactgagcggcggacgggtgagtaacgcgtgggtaacctgcctcatacagggggataacagcta |
| | | gaaatggctgctaataccgcataagaccacagtactgcatggtacagtggtaaaaactccggtggtatgagatggacccgcgt |
| | | ctgattaggtagttggtgaggtaacggcccaccaagccgacgatcagtagccgacctgagagggtgaccggccacattggg |
| | | actgagacacggcccagactcctacgggaggcagcagtggggaatattgcacaatgggcgaaagcctgatgcagcgacgc |
| | | cgcgtgaaggatgaagtatttcggtatgtaaacttctatcagcagggaagaaaatgacggtacctgactaagaagccccggct |
| | | aactacgtgccagcagccgcggtaatacgtagggggcaagcgttatccggatttactgggtgtaaagggagcgtagacggct |
| | | gtgcaagtctgaagtgaaaggcatgggctcaacctgtggactgctttggaaactgtgcagctagagtgtcggagaggtaagtg |
| | | gaattcctagtgtagcggtgaaatgcgtagatattaggaggaacaccagtggcgaaggcggcttactggacgatgactgacgt |
| | | tgaggctcgaaagcgtggggagcaaacaggattagataccctggtagtccacgccgtaaacgatgactgctaggtgtcgggt |
| | | agcaaagctattcggtgccgcagctaacgcaataagcagtccacctggggagtacgttcgcaagaatgaaactcaaaggaatt |
| | | gacggggacccgcacaagcggtggagcatgtggtttaattcgaagcaacgcgaagaaccttacctgatcttgacatcccgatg |
| | | accgcttcgtaatggaagtttttcttcggaacatcggtgacaggtggtgcatggttgtcgtcagctcgtgtcgtgagatgttgggtt |
| | | aagtcccgcaacgagcgcaacccttatcttcagtagccagcattaaggatgggcactctggagagactgccagggataacctg |
| | | gaggaaggtggggatgacgtcaaatcatcatgccccttatgaccagggctacacacgtgctacaatggcgtaaacaaaggga |
| | | ggcagagccgcgaggccgagcaaatctcaaaaataacgtctcagttcggattgtagtctgcaactcgactacatgaagctgga |
| | | atcgctagtaatcgcagatcagaatgctgcggtgaatacgttcccgggtcttgtacacaccgcccgtcacaccatgggagtcag |
| | | taacgcccgaagtcagtgacccaaccgaaaggagggagctgccgaaggtgggaccgataactggggtgaagtcgtaacaa |
| | | ggtagccgtatcggaaggtgcggctggatcacctccttt |
| Dorea_ | SEQ ID | acgagagtttgatcctggctcaggatgaacgctggcggcgtgcttaacacatgcaagtcgagcgaagcacttaagtttgattctt |
| formicigenerans | NO: 115 | cggatgaagacttttgtgactgagcggcggacgggtgagtaacgcgtgggtaacctgcctcatacagggggataacagttag |
| | | aaatggctgctaataccgcataagaccacagtactgcatggtacagtggtaaaaactccggtggtatgagatggacccgcgtct |
| | | gattaggtagttggtgaggtaacggcccaccaagccgacgatcagtagccgacctgagagggtgaccggccacattgggac |
| | | tgagacacggcccagactcctacgggaggcagcagtggggaatattgcacaatgggcgaaagcctgatgcagcgacgccg |
| | | cgtgaaggatgaagtatttcggtatgtaaacttctatcagcagggaagaaaatgacggtacctgactaagaagccccggctaac |
| | | tacgtgccagcagccgcggtaatacgtagggggcaagcgttatccggatttactgggtgtaaagggagcgtagacggctgtg |
| | | caagtctgaagtgaaaggcatgggctcaacctgtggactgctttggaaactgtgcagctagagtgtcggagaggtaagtggaa |
| | | ttcctagtgtagcggtgaaatgcgtagatattaggaggaacaccagtggcgaaggcggcttactggacgatgactgacgttga |
| | | ggctcgaaagcgtggggagcaaacaggattagataccctggtagtccacgccgtaaacgatgactgctaggtgtcgggtagc |
| | | aaagctattcggtgccgcagctaacgcaataagcagtccacctggggagtacgttcgcaagaatgaaactcaaaggaattgac |
| | | ggggacccgcacaagcggtggagcatgtggtttaattcgaagcaacgcgaagaaccttacctgatcttgacatcccgatgacc |
| | | gatcgtaatggaagcttttcttcggaacatcggtgacaggtggtgcatggttgtcgtcagctcgtgtcgtgagatgttgggttaa |
| | | gtcccgcaacgagcgcaacccttatcttcagtagccagcattaaggatgggcactctggagagactgccagggataacctgg |
| | | aggaaggtggggatgacgtcaaatcatcatgccccttatgaccagggctacacacgtgctacaatggcgtaaacaaagggaa |
| | | gcagagccgcgaggccgagcaaatctcaaaaataacgtctcagttcggattgtagtctgcaactcgactacatgaagctggaa |
| | | tcgctagtaatcgcagatcagaatgctgcggtgaatacgttcccgggtcttgtacacaccgcccgtcacaccatgggagtcagt |
| | | aacgcccgaagtcagtgacccaaccgaaaggagggagctgccgaaggtgggaccgataactggggtgaagtcgtaacaa |
| | | ggtagccgtatcggaaggtgcggctggatcacctccttt |
| Dorea_ | SEQ ID | acgagagtttgatcctggctcaggatgaacgctggcggcgtgcttaacacatgcaagtcgagcgaagcacttaagtttgattctt |
| formicigenerans | NO: 116 | cggatgaagacttttgtgactgagcggcggacgggtgagtaacgcgtgggtaacctgcctcatacagggggataacagttag |
| | | aaatggctgctaataccgcataagaccacagtactgcatggtacagtggtaaaaactccggtggtatgagatggacccgcgtct |
| | | gattaggtagttggtgaggtaacggcccaccaagccgacgatcagtagccgacctgagagggtgaccggccacattgggac |
| | | tgagacacggcccagactcctacgggaggcagcagtggggaatattgcacaatgggcgaaagcctgatgcagcgacgccg |
| | | cgtgaaggatgaagtatttcggtatgtaaacttctatcagcagggaagaaaatgacggtacctgactaagaagccccggctaac |
| | | tacgtgccagcagccgcggtaatacgtagggggcaagcgttatccggatttactgggtgtaaagggagcgtagacggctgtg |
| | | caagtctgaagtgaaaggcatgggctcaacctgtggactgctttggaaactgtgcagctagagtgtcggagaggtaagtggaa |
| | | ttcctagtgtagcggtgaaatgcgtagatattaggaggaacaccagtggcgaaggcggcttactggacgatgactgacgttga |
| | | ggctcgaaagcgtggggagcaaacaggattagataccctggtagtccacgccgtaaacgatgactgctaggtgtcgggtagc |
| | | aaagctattcggtgccgcagctaacgcaataagcagtccacctggggagtacgttcgcaagaatgaaactcaaaggaattgac |
| | | ggggacccgcacaagcggtggagcatgtggtttaattcgaagcaacgcgaagaaccttacctgatcttgacatcccgatgacc |
| | | gcttcgtaatggaagtttttcttcggaacatcggtgacaggtggtgcatggttgtcgtcagctcgtgtcgtgagatgttgggttaag |
| | | tcccgcaacgagcgcaacccttatattcagtagccagcatttaggatgggcactctggagagactgccagggataacctggag |
| | | gaaggtggggatgacgtcaaatcatcatgccccttatgaccagggctacacacgtgctacaatggcgtaaacaaagggaggc |
| | | agagccgcgaggccgagcaaatctcaaaaataacgtctcagttcggattgtagtctgcaactcgactacatgaagctggaatc |
| | | gctagtaatcgcagatcagaatgctgcggtgaatacgttcccgggtcttgtacacaccgcccgtcacaccatgggagtcagtaa |
| | | cgcccgaagtcagtgacccaaccgaaaggagggagctgccgaaggtgggaccgataactggggtgaagtcgtaacaaggt |
| | | agccgtatcggaaggtgcggctggatcacctccttt |
| Dorea_ | SEQ ID | acgagagtttgatcctggctcaggatgaacgctggcggcgtgcttaacacatgcaagtcgagcgaagcacttaagtttgattctt |
| formicigenerans | NO: 117 | cggatgaagacttttgtgactgagcggcggacgggtgagtaacgcgtgggtaacctgcctcatacagggggataacagttag |
| | | aaatggctgctaataccgcataagaccacagtactgcatggtacagtggtaaaaactccggtggtatgagatggacccgcgtct |
| | | gattaggtagttggtgaggtaacggcccaccaagccgacgatcagtagccgacctgagagggtgaccggccacattgggac |
| | | tgagacacggcccagactcctacgggaggcagcagtggggaatattgcacaatgggcgaaagcctgatgcagcgacgccg |
| | | cgtgaaggatgaagtatttcggtatgtaaacttctatcagcagggaagaaaatgacggtacctgactaagaagccccggctaac |
| | | tacgtgccagcagccgcggtaatacgtagggggcaagcgttatccggatttactgggtgtaaagggagcgtagacggctgtg |
| | | caagtctgaagtgaaaggcatgggctcaacctgtggactgctttggaaactgtgcagctagagtgtcggagaggtaagtggaa |
| | | ttcctagtgtagcggtgaaatgcgtagatattaggaggaacaccagtggcgaaggcggcttactggacgatgactgacgttga |
| | | ggctcgaaagcgtggggagcaaacaggattagataccctggtagtccacgccgtaaacgatgactgctaggtgtcgggtagc |
| | | aaagctattcggtgccgcagctaacgcaataagcagtccacctggggagtacgttcgcaagaatgaaactcaaaggaattgac |
| | | ggggacccgcacaagcggtggagcatgtggtttaattcgaagcaacgcgaagaaccttacctgatcttgacatcccgatgacc |
| | | gcttcgtaatggaagcttttcttcggaacatcggtgacaggtggtgcatggttgtcgtcagctcgtgtcgtgagatgttgggttaa |
| | | gtcccgcaacgagcgcaacccttatcttcagtagccagcatttaggatgggcactctggagagactgccagggataacctgga |
| | | ggaaggtggggatgacgtcaaatcatcatgccccttatgaccagggctacacacgtgctacaatggcgtaaacaaagggagg |
| | | cagagccgcgaggccgagcaaatctcaaaaataacgtctcagttcggattgtagtctgcaactcgactacatgaagctggaatc |
| | | gctagtaatcgcagatcagaatgctgcggtgaatacgttcccgggtcttgtacacaccgcccgtcacaccatgggagtcagtaa |
| | | cgcccgaagtcagtgacccaaccgaaaggagggagctgccgaaggtgggaccgataactggggtgaagtcgtaacaaggt |
| | | agccgtatcggaaggtgcggctggatcacctccttt |
| Dorea_ | SEQ ID | tatgagatggacccgcgtctgattaggtagttggtgaggtaacggcccaccaagccgacgatcagtagccgacctgagaggg |
| formicigenerans | NO: 118 | tgaccggccacattgggactgagacacggcccagactcctacgggaggcagcagtggggaatattgcacaatgggcgaaa |
| | | gcctgatgcagcgacgccgcgtgaaggatgaagtatttcggtatgtaaacttctatcagcagggaagaaaatgacggtacctg |
| | | actaagaagccccggctaactacgtgccagcagccgcggtaatacgtagggggcaagcgttatccggatttactgggtgtaa |
| | | agggagcgtagacggctgtgcaagtctgaagtgaaaggcatgggctcaacctgtggactgctttggaaactgtgcagctaga |
| | | gtgtcggagaggtaagtggaattcctagtgtagcggtgaaatgcgtagatattaggaggaacaccagtggcgaaggcggctt |
| | | actggacgatgactgacgttgaggctcgaaagcgtggggagcaaacaggattagataccctggtagtccacgccgtaaacga |
| | | tgactgctaggtgtcgggtagcaaagctattcggtgccgcagctaacgcaataagcagtccacctggggagtacgttcgcaag |
| | | aatgaaactcaaaggaattgacggggacccgcacaagcggtggagcatgtggtttaattcgaagcaacgcgaagaaccttac |
| | | ctgatcttgacatcccgatgaccgcttcgtaatggaagtttttcttcggaacatcggtgacaggtggtgcatggttgtcgtcagctc |
| | | gtgtcgtgagatgttgggttaagtcccgcaacgagcgcaacccttatcttcagtagccagcatttaggatgggcactctggaga |
| | | gactgccagggataacctggaggaaggtggggatgacgtcaaatcatcatgccccttatgaccagggctacacacgtgctac |
| | | aatggcgtaaacaaagggaggcagagccgcgaggccgagcaaatctcaaaaataacgtctcagttcggattgtagtctgcaa |
| | | ctcgactacatgaagctggaatcgctagtaatcgcagatcagaatgctgcggtgaatacgttcccgggtcttgtacacaccgcc |
| | | cgtcacaccatgggagtcagtaacgcccgaagtcagtgacccaaccgaaaggagggagctgccgaaggtgggaccgataa |
| | | ctggggtgaagtcgtaacaaggtagccgtatcggaaggtgcggctggatcacctccttt |
| Dorea_ | SEQ ID | acgagagtttgatcctggctcaggatgaacgctggcggcgtgcttaacacatgcaagtcgagcgaagcacataagttcgattct |
| formicigenerans | NO: 119 | tcggatgaagacttttgtgactgagcggcggacgggtgagtaacgcgtgggtaacctgcctcatacagggggataacagcta |
| | | gaaatggctgctaataccgcataagaccacagtactgcatggtacagtggtaaaaactccggtggtatgagatggacccgcgt |
| | | ctgattaggtagttggtgaggtaacggcccaccaagccgacgatcagtagccgacctgagagggtgaccggccacattggg |
| | | actgagacacggcccagactcctacgggaggcagcagtggggaatattgcacaatgggcgaaagcctgatgcagcgacgc |
| | | cgcgtgaaggatgaagtatttcggtatgtaaacttctatcagcagggaagaaaatgacggtacctgactaagaagccccggct |
| | | aactacgtgccagcagccgcggtaatacgtagggggcaagcgttatccggatttactgggtgtaaagggagcgtagacggct |
| | | gtgcaagtctgaagtgaaaggcatgggctcaacctgtggactgctttggaaactgtgcagctagagtgtcggagaggtaagtg |
| | | gaattcctagtgtagcggtgaaatgcgtagatattaggaggaacaccagtggcgaaggcggcttactggacgatgactgacgt |
| | | tgaggctcgaaagcgtggggagcaaacaggattagataccctggtagtccacgccgtaaacgatgactgctaggtgtcgggt |
| | | agcaaagctattcggtgccgcagctaacgcaataagcagtccacctggggagtacgttcgcaagaatgaaactcaaaggaatt |
| | | gacggggacccgcacaagcggtggagcatgtggtttaattcgaagcaacgcgaagaaccttacctgatcttgacatcccgatg |
| | | accgcttcgtaatggaagcttttcttcggaacatcggtgacaggtggtgcatggttgtcgtcagctcgtgtcgtgagatgttgggt |
| | | taagtcccgcaacgagcgcaacccttatcttcagtagccagcatttaggatgggcactctggagagactgccagggataacct |
| | | ggaggaaggtggggatgacgtcaaatcatcatgccccttatgaccagggctacacacgtgctacaatggcgtaaacaaaggg |
| | | aagcagagccgcgaggccgagcaaatctcaaaaataacgtctcagttcggattgtagtctgcaactcgactacatgaagctgg |
| | | aatcgctagtaatcgcagatcagaatgctgcggtgaatacgttcccgggtcttgtacacaccgcccgtcacaccatgggagtca |
| | | gtaacgcccgaagtcagtgacccaaccgaaaggagggagctgccgaaggtgggaccgataactggggtgaagtcgtaaca |
| | | aggtagccgtatcggaaggtgcggctggatcacctccttt |
| Eubacterium_ | SEQ ID | ctgagtggcggacgggtgagtaacgcgtgggtaacctgccctgtacagggggataacagaggaaacggctgctaataccg |
| hallii | NO: 120 | cataagcgcacgaggagacatctccttgtgtgaaaaactccggtggtacaggatgggcccgcgtctgattagaggttggcag |
| | | ggtaacggcctaccaaggcaacgatcagtagccggtctgagaggatgaacggccacattggaactgagacacggtccaaac |
| | | tcctacgggaggcagcagtggggaatattgcacaatgggggaaaccctgatgcagcaacgccgcgtgagtgaagaagtattt |
| | | cggtatgtaaagctctatcagcagggaagataatgacggtacctgactaagaagaccggctaaatacgtgccagcagccgc |
| | | ggtaatacgtatggagcaagcgttatccggatttactgggtgtaaagggtgcgtaggtggcagtgcaagtcagatgtgaaagg |
| | | ccggggctcaaccccggagctgcatttgaaactgctcggctagagtacaggagaggcaggcggaattcctagtgtagcggtg |
| | | aaatgcgtagatattaggaggaacaccagtggcgaaggcggcctgctggactgttactgacactgaggcacgaaagcgtgg |
| | | ggagcaaacaggattagataccctggtagtccacgccgtaaacgatgaatactaggtgtcggggccgtataggcttcggtgcc |
| | | gccgctaacgcagtaagtattccacctggggagtacgttcgcaagaatgaaactcaaaggaattgacggggacccgcacaag |
| | | cggtggagcatgtggtttaattcgaagcaacgcgaagaaccttaccaggtcttgacatccttctgaccgcaccttaatcggtgctt |
| | | tccttcgggacagaagagacaggtggtgcatggttgtcgtcagctcgtgtcgtgagatgttgggttaagtcccgcaacgagcg |
| | | caacccctatcttcagtagccagcaggtaaggctgggcactctggagagactgccagggataacctggaggaaggtgggga |
| | | cgacgtcaaatcatcatgccccttatgatctgggcgacacacgtgctacaatggcggtcacagagtgaggcgaacccgcgag |
| | | ggggagcaaaccacaaaaaggccgtcccagttcggactgtagtctgcaacccgactacacgaagctggaatcgctagtaatc |
| | | gcgaatcagaatgtcgcggtgaatacgttcccgggtcttgtacacaccgcccgtcacaccatgggagtcggaaatgcccgaa |
| | | gccagtgacccaaccttttggagggagctgtcgaaggtggagccggtaactggggtgaagtcgtaacaaggtagccgtatcg |
| | | gaaggtgcggctggatcacctccttt |
| Ruminococcaceae_ | SEQ ID | tattgagagtttgatcctggctcaggatgaacgctggcggcgtgcttaacacatgcaagtcgaacggagtgcctttgaaagagg |
| bacterium_ | NO: 121 | attcgtccaattgataaggttacttagtggcggacgggtgagtaacgcgtgaggaacctgccttggagtggggaataacacagt |
| D16 | | gaaaattgtgctaataccgcataatgcagttgggccgcatggctctgactgccaaagatttatcgctctgagatggcctcgcgtc |
| | | tgattagctagttggtggggtaacggcccaccaaggcgacgatcagtagccggactgagaggttggccggccacattggga |
| | | ctgagacacggcccagactcctacgggaggcagcagtggggaatattgggcaatgggcgcaagcctgacccagcaacgcc |
| | | gcgtgaaggaagaaggctttcgggttgtaaacttcttttcttagggacgaagcaagtgacggtacctaaggaataagccacgg |
| | | ctaactacgtgccagcagccgcggtaatacgtaggtggcaagcgttatccggatttactgggtgtaaagggcgtgtaggcggg |
| | | attgcaagtcagatgtgaaaaccacgggctcaacctgtggcctgcatttgaaactgtagttcttgagtactggagaggcagacg |
| | | gaattcctagtgtagcggtgaaatgcgtagatattaggaggaacaccagtggcgaaggcggtctgctggacagcaactgacg |
| | | ctgaggcgcgaaagcgtggggagcaaacaggattagataccctggtagtccacgctgtaaacgatggatactaggtgtggg |
| | | gggtctgaccccttccgtgccgcagttaacacaataagtatcccacctggggagtacgatcgcaaggttgaaactcaaaggaa |
| | | ttgacgggggcccgcacaagcggtggagtatgtggtttaattcgaagcaacgcgaagaaccttaccagggcttgacatcccg |
| | | aggcccggtctagagatagacctttctcttcggagacctcggtgacaggtggtgcatggttgtcgtcagctcgtgtcgtgagatg |
| | | ttgggttaagtcccgcaacgagcgcaacccctattgttagttgctacgcaagagcactctagcgagactgccgttgacaaaacg |
| | | gaggaaggtggggacgacgtcaaatcatcatgccccttatgtcctgggccacacacgtactacaatggtggttaacagaggg |
| | | aggcaataccgcgaggtggagcaaacccctaaaagccatcccagttcggattgcaggctgcaacccgcctgcatgaagttgg |
| | | aatcgctagtaatcgcggatcagcatgccgcggtgaatacgttcccgggccttgtacacaccgcccgtcacaccatgagagtc |
| | | gggaacacccgaagtccgtagcctaaccgcaaggggggcgcggccgaaggtgggttcgataattggggtgaagtcgtaac |
| | | aaggtagccgtatcggaaggtgcggctggatcacctccttt |
| Streptococcus_ | SEQ ID | aatgagagtttgatcctggctcaggacgaacgctggcggcgtgcctaatacatgcaagtagaacgctgaagagaggagcttg |
| vestibularis | NO: 122 | ctcttcttgaatgagttgcgaacgggtgagtaacgcgtaggtaacctgccttgtagcgggggataacattggaaacgatagcta |
| | | ataccgcataacaatgggtgacacatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtaggaatcttcggcaatgggggcaaccctgaccgagcaacgccgcgtgagtgaa |
| | | gaaggttttcggatcgtaaagctctgttgtaagtcaagaacgagtgtgagagtggaaagttcacactgtgacggtagcttaaccg |
| | | agaaagggacggctaactacgtgccagcagccgcggtaatacgtaggtcccgagcgttgtccggatttattgggcgtaaagc |
| | | gagcgcaggcggtttgataagtctgaagttaaaggctgtggctcaaccatagttcgctttggaaactgtcaaacttgagtgcaga |
| | | aggggagagtggaattccatgtgtagcggtgaaatgcgtagatatatggaggaacaccggtggcgaaagcggctctctggtc |
| | | tgtaactgacgctgaggctcgaaagcgtgggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagaccaatctcagttcggattgtaggctgcaactcgcctacat |
| | | gaagtcggaatcgctagtaatcgcggatcagcacgccgcggtgaatacgttcccgggccttgtacacaccgcccgtcacacc |
| | | acgagagtttgtaacacccgaagtcggtgaggtaaccttttggagccagccgcctaaggtgggatagatgattggggtgaagt |
| | | cgtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| vestibularis | NO: 123 | ctcttcttgaatgagttgcgaacgggtgagtaacgcgtaggtaacctgccttgtagcgggggataactattggaaacgatagcta |
| | | ataccgcataacaatgggtgacacatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| vestibularis | NO: 124 | ctcttcttgaatgagttgcgaacgggtgagtaacgcgtaggtaacctgccttgtagcgggggataactattggaaacgatagcta |
| | | ataccgcataacaatgggtgacacatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgtcgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagtaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccgt |
| | | aataaaccggaggaaggtggggatgacgtcaaatcatcatgccccttatgacctgggctacacacgtgctacaatggttggta |
| | | caacgagttgcgagtcggtgacggcaagctaatctcttaaagccaatctcagttcggattgtaggctgcaactcgcctacatga |
| | | agtcggaatcgctagtaatcgcggatcagcacgccgcggtgaatacgttcccgggccttgtacacaccgcccgtcacaccac |
| | | gagagtttgtaacacccgaagtcggtgaggtaaccttttggagccagccgcctaaggtggggtagatgattggggtgaagtcg |
| | | taacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| vestibularis | NO: 125 | ctcttcttgaatgagttgcgaacgggtgagtaacgcgtaggtaacctgccttgtagcgggggataactattggaaacgatagcta |
| | | ataccgcataacaatggtgacacatgtcattatttgaaaggggcaattgctccactacaagatggacctgcgttgtattagctagt |
| | | aggtgaggtaacggctcacctaggcgacgatacatagccgacctgagagggtgatcggccacactgggactgagacacgg |
| | | cccagactcctacgggaggcagcagtagggaatcttcggcaatgggggcaaccctgaccgagcaacgccgcgtgagtgaa |
| | | gaaggttttcggatcgtaaagctctgttgtaagtcaagaacgagtgtgagagtggaaagttcacactgtgacggtagcttaccag |
| | | aaagggacggctaactacgtgccagcagccgcggtaatacgtaggtcccgagcgttgtccggatttattgggcgtaaagcga |
| | | gcgcaggcggtttgataagtctgaagttaaaggctgtggctcaaccatagttcgctttggaaactgtcaaacttgagtgcagaag |
| | | gggagagtggaattccatgtgtagcggtgaaatgcgtagatatatggaggaacaccggtggcgaaagcggctctctggtctgt |
| | | aactgacgctgaggctcgaaagcgtggggagcgaacaggattagataccctggtagtccacgccgtaaacgatgagtgcta |
| | | ggtgttggatcctttccgggattcagtgccgcagctaacgcattaagcactccgcctggggagtacgaccgcaaggttgaaact |
| | | caaaggaattgacgggggcccgcacaagcggtggagcatgtggtttaattcgaagcaacgcgaagaaccttaccaggtcttg |
| | | acatcccgatgctatttctagagatagaaagttacttcggtacatcggtgacaggtggtgcatggagtcgtcagctcgtgtcgtg |
| | | agatgttgggttaagtcccgcaacgagcgcaacccctattgttagttgccatcattcagttgggcactctagcgagactgccggt |
| | | aataaaccggaggaaggtggggatgacgtcaaatcatcatgccccttatgacctgggctacacacgtgctacaatggttggta |
| | | caacgagttgcgagtcggtgacggcaagctaatctcttaaagccaatctcagttcggattgtaggctgcaactcgcctacatga |
| | | agtcggaatcgctagtaatcgcggatcagcacgccgcggtgaatacgttcccgggccttgtacacaccgcccgtcacaccac |
| | | gagagtttgtaacacccgaagtcggtgaggtaaccttttggagccagccgcctaaggtgggatagatgattggggtgaagtcg |
| | | taacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| vestibularis | NO: 126 | ctcttcttgaatgagttgcgaacgggtgagtaacgcgtaggtaacctgccttgtagcgggggataactattggaaacgatagcta |
| | | ataccgcataacaatgggtgacacatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| vestibularis | NO: 127 | ctcttcttgaatgagttgcgaacgggtgagtaacgcgtaggtaacctgccttgtagcgggggataactattggaaacgatagcta |
| | | ataccgcataacaatgggtgacacatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| thermophilus | NO: 128 | ctcttcttggatgagttgcgaacgggtgagtaacgcgtaggtaacctgccttgtagcgggggataactattggaaacgatagct |
| | | aataccgcataacaatggatgacacatgtcatttatttgaaaggggcaattgctccactacaagatggacctgcgttgtattagct |
| | | agtaggtgaggtaatggctcacctaggcgacgatacatagccgacctgagagggtgatcggccacactgggactgagacac |
| | | ggcccagactcctacgggaggcagcagtagggaatcttcggcaatgggggcaaccctgaccgagcaacgccgcgtgagtg |
| | | aagaaggttttcggatcgtaaagctctgttgtaagtcaagaacgggtgtgagagtggaaagttcacactgtgacggtagcttacc |
| | | agaaagggacggctaactacgtgccagcagccgcggtaatacgtaggtcccgagcgttgtccggatttattgggcgtaaagc |
| | | gagcgcaggcgggttgataagtctgaagttaaaggctggggctcaaccatagttcgctttggaaactgtcaaacttgagtgcag |
| | | aaggggagagtggaattccatgtgtagcggtgaaatgcgtagatatatggaggaacaccggtggcgaaagcggctctctggt |
| | | ctgtaactgacgctgaggctcgaaagcgtggggagcgaacaggattagataccctggtagtccacgccgtaaacgatgagtg |
| | | ctaggtgaggatcctttccgggattcagtgccgaagctaacgcattaagcactccgcctggggagtacgaccgcaaggttgaa |
| | | actcaaaggaattgacgggggcccgcacaagcggtggagcatgtggtttaattcgaagcaacgcgaagaaccttaccaggtc |
| | | ttgacatcccgatgctatttctagagatagaaagttacttcggtacatcggtgacaggtggtgcatggttgtcgtcagctcgtgtcg |
| | | tgagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcgagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggacagatgattggggtgaagtc |
| | | gtaacaaggtagccgtatcggaaggtgcggctgggtcacctcctt |
| Streptococcus_ | SEQ ID | atgaaagtttgatcctggctcaggacgaacgctggcggcgtgcctaatacatgcaagtagaacgctgaagagaggagcttgct |
| thermophilus | NO: 129 | cttcttggatgagttgcgaacgggtgagtaacgcgtaggtaacctgccttgtagcgggggataactattggaaacgatagctaa |
| | | taccgcataacaatggatgacacatgtcatttatttgaaaggggcaattgctccactacaagatggacctgcgttgtattagctagt |
| | | aggtgaggtaatggctcacctaggcgacgatacatagccgacctgagagggtgatcggccacactgggactgagacacggc |
| | | ccagactcctacgggaggcagcagtagggaatcttcggcaatgggggcaaccctgaccgagcaacgccgcgtgagtgaag |
| | | aaggttttcggatcgtaaagctctgttgtaagtcaagaacgggtgtgagagtggaaagttcacactgtgacggtagcttaccaga |
| | | aagggacggctaactacgtgccagcagccgcggtaatacgtaggtcccgagcgttgtccggatttattgggcgtaaagcgag |
| | | cgcaggcggtttgataagtctgaagttaaaggctgtggctcaaccatagttcgctttggaaactgtcaaacttgagtgcagaagg |
| | | ggagagtggaattccatgtgtagcggtgaaatgcgtagatatatggaggaacaccggtggcgaaagcggctcactggtctgta |
| | | actgacgctgaggctcgaaagcgtggggagcgaacaggattagataccctggtagtccacgccgtaaacgatgagtgctag |
| | | gtgttggatcctttccgggattcagtgccgcagctaacgcattaagcactccgcctggggagtacgaccgcaaggttgaaactc |
| | | aaaggaattgacgggggcccgcacaagcggtggagcatgtggtttaattcgaagcaacgcgaagaaccttaccaggtcttga |
| | | catcccgatgctatttctagagatagaaagttacttcggtacatcggtgacaggtggtgcatggttgtcgtcagctcgtgtcgtga |
| | | gatgttgggttaagtcccgcaacgagcgcaacccctattgttagttgccatcattcagttgggcactctagcgagactgccggta |
| | | ataaaccggaggaaggtggggatgacgtcaaatcatcatgccccttatgacctgggctacacacgtgctacaatggttggtac |
| | | aacgagttgcgagtcggtgacggcgagctaatctcttaaagccaatctcagttcggattgtaggctgcaactcgcctacatgaa |
| | | gtcggaatcgctagtaatcgcggatcagcacgccgcggtgaatacgttcccgggccttgtacacaccgcccgtcacaccacg |
| | | agagtttgtaacacccgaagtcggtgaggtaaccttttggagccagccgcctaaggtgggacagatgattggggtgaagtcgt |
| | | aacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| thermophilus | NO: 130 | ctcttcttggatgagttgcgaacgggtgagtaacgcgtaggtaacctgccttgtagcgggggataactattggaaacgatagct |
| | | aataccgcataacaatggatgacacatgtcatttatttgaaaggggcaattgctccactacaagatggacctgcgttgtattagct |
| | | agtaggtgaggtaatggctcacctaggcgacgatacatagccgacctgagagggtgatcggccacactgggactgagacac |
| | | ggcccagactcctacgggaggcagcagtagggaatcttcggcaatgggggcaaccctgaccgagcaacgccgcgtgagtg |
| | | aagaaggttttcggatcgtaaagctctgttgtaagtcaagaacgggtgtgagagtggaaagttcacactgtgacggtagcttacc |
| | | agaaagggacggctaactacgtgccagcagccgcggtaatacgtaggtcccgagcgttgtccggatttattgggcgtaaagc |
| | | gagcgcaggcggtttgataagtctgaagttaaaggctgtggctcaaccatagttcgctttggaaactgtcaaacttgagtgcaga |
| | | aggggagagtggaattccatgtgtagcggtgaaatgcgtagatatatggaggaacaccggtggcgaaagcggctctctggtc |
| | | tgtaactgacgctgaggctcgaaagcgtggggagcgaacaggattagataccctggtagtccacgccgtaaacgatgagtgc |
| | | taggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcgagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggacagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| thermophilus | NO: 131 | ctcttcttggatgagttgcgaacgggtgagtaacgcgtaggtaacctgccttgtagcgggggataactattggaaacgatagct |
| | | aataccgcataacaatggatgacacatgtcatttatttgaaaggggcaattgctccactacaagatggacctgcgttgtattagct |
| | | agtaggtgaggtaatggctcacctaggcgacgatacatagccgacctgagagggtgatcggccacactgggactgagacac |
| | | ggcccagactcctacgggaggcagcagtagggaatcttcggcaatgggggcaaccctgaccgagcaacgccgcgtgagtg |
| | | aagaaggttttcggatcgtaaagctctgttgtaagtcaagaacgggtgtgagagtggaaagttcacactgtgacggtagcttacc |
| | | agaaagggacggctaactacgtgccagcagccgcggtaatacgtaggtcccgagcgttgtccggatttattgggcgtaaagc |
| | | gagcgcaggcgggttgataagtctgaagttaaaggctggggctcaaccatagttcgctttggaaactgtcaaacttgagtgcag |
| | | aaggggagagtggaattccatgtgtagcggtgaaatgcgtagatatatggaggaacaccggtggcgaaagcggctctctggt |
| | | ctgtaactgacgctgaggctcgaaagcgtggggagcgaacaggattagataccctggtagtccacgccgtaaacgatgagtg |
| | | ctaggtgaggatcctttccgggattcagtgccgcagctaacgcattaagcactccgcctggggagtacgaccgcaaggttgaa |
| | | actcaaaggaattgacgggggcccgcacaagcggtggagcatgtggtttaattcgaagcaacgcgaagaaccttaccaggtc |
| | | ttgacatcccgatgctatttctagagatagaaagttacttcggtacatcggtgacaggtggtgcatggttgtcgtcagctcgtgtcg |
| | | tgagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcgagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggacagatgattggggtgaagtc |
| | | gtaacaaggtagccgtatcggaaggtgcggctggtcacctcctt |
| Streptococcus_ | SEQ ID | aatgagagtttgatcctggctcaggacgaacgctggcggcgtgcctaatacatgcaagtagaacgctgaagagaggagcttg |
| thermophilus | NO: 132 | ctcttcttggatgagttgcgaacgggtgagtaacgcgtaggtaacctgccttgtagcgggggataactattggaaacgatagct |
| | | aataccgcataacaatggatgacacatgtcatttatttgaaaggggcaattgctccactacaagatggacctgcgttgtattagct |
| | | agtaggtgaggtaatggctcacctaggcgacgatacatagccgacctgagagggtgatcggccacactgggactgagacac |
| | | ggcccagactcctacgggaggcagcagtagggaatcttcggcaatgggggcaaccctgaccgagcaacgccgcgtgagtg |
| | | aagaaggttttcggatcgtaaagctctgttgtaagtcaagaacgggtgtgagagtggaaagttcacactgtgacggtagcttacc |
| | | agaaagggacggctaactacgtgccagcagccgcggtaatacgtaggtcccgagcgttgtccggatttattgggcgtaaagc |
| | | gagcgcaggcgggttgataagtctgaagttaaaggctgtggctcaaccatagttcgctttggaaactgtcaaacttgagtgcag |
| | | aaggggagagtggaattccatgtgtagcggtgaaatgcgtagatatatggaggaacaccggtggcgaaagcggctctctggt |
| | | ctgtaactgacgctgaggctcgaaagcgtggggagcgaacaggattagataccctggtagtccacgccgtaaacgatgagtg |
| | | ctaggtgttggatcctttccgggattcagtgccgcagctaacgcattaagcactccgcctggggagtacgaccgcaaggttgaa |
| | | actcaaaggaattgacgggggcccgcacaagcggtggagcatgtggtttaattcgaagcaacgcgaagaaccttaccaggtc |
| | | ttgacatcccgatgctatttctagagatagaaagttacttcggtacatcggtgacaggtgggcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcgagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggacagatgattggggtgaagtc |
| | | gtaacaaggtagccgtatcggaaggtgcggctgggtcacctcctt |
| Prevotella_ | SEQ ID | cgtatccaaccttcccgcgaccaagggataacctgccgaaaggcagactaataccttatgtcctcctttgaaggcatctgacga |
| buccae | NO: 133 | ggagcaaagatccgtcggtcacggatggggatgcgtccgattagcttgaggcggggcaacggcccaccaaggcttcgatc |
| | | ggtaggggttctgagaggaaggccccccacactggaactgagacacggtccagactcctacgggaggcagcagtgaggaa |
| | | tattggtcaatgggcggaagcctgaaccagccaagtagcgtgcaggatgacggccctacgggttgtaaactgcttttatgcgg |
| | | ggataaagttgcccacgcgtgggtttttgcaggtaccgcatgaataaggaccggctaattccgtgccagcagccgcggtaata |
| | | cggaaggtccgggcgttatccggatttattgggtttaaagggagcgcaggccgccgtgcaagcgtgccgtgaaaagcagcg |
| | | gcccaaccgctgccctgcggcgcgaactgcttggcttgagtgcgccggaagcgggcggaattcgtggtgtagcggtgaaat |
| | | gcttagatatcacgaagaaccccgattgcgaaggcagcccgctgtggcgccactgacgctgaggctcgaaggtgcgggtat |
| | | cgaacaggattagataccctggtagtccgcacggtaaacgatggatacccgctgtccggcccgtacggtctgggcggccaag |
| | | cgaaagcgttaagtatcccacctggggagtacgccggcaacggtgaaactcaaaggaattgacgggggcccgcacaagcg |
| | | gaggaacatgtggtttaattcgatgatacgcgaggaaccttacccgggcttgaattgcaggagaatgattcagagatgatgaag |
| | | tccctcgggactcctgtgaaggtgctgcatggagtcgtcagctcgtgccgtgaggtgtcggctcaagtgccataacgagcgca |
| | | acccctctccgcagttgccatcgggtcatgccgggcactctgcggacactgccgccgcaaggtgcgaggaaggtggggatg |
| | | acgtcaaatcagcacggcccttacgtccggggccacacacgtgttacaatggccggcacagagggctgtccgcatgcaagtg |
| | | cgggcgaatccccaaatccggtcccagttcggactggggtctgcaacccgaccccacgaagctggattcgctagtaatcgcg |
| | | catcagccatggcgcggtgaatacgttcccgggccttgtacacaccgcccgtcaagccatgaaagccgggggtgcctgaagt |
| | | ccgtgaccgcgagggtcggcctagggcaaaaccggtgattggggctaagtcgtaacaaggtagccgtaccggaaggtgcg |
| | | gctggaacacctccttt |
| Lachnospiraceae_ | SEQ ID | atgagagtttgatcctggctcaggatgaacgctggcggcgtgcttaacacatgcaagtcgaacgaaacaccttatttgattttctt |
| bacterium_ | NO: 134 | cggaactgaagatttggtgattgagtggcggacgggtgagtaacgcgtgggtaacctgccctgtacagggggataacagtca |
| 5_1_63 | | gaaatgactgctaataccgcataagaccacagcaccgcatggtgcaggggtaaaaactccggtggtacaggatggacccgc |
| FAA | | gtctgattagctggttggtgaggtaacggctcaccaaggcgacgatcagtagccggcttgagagagtgaacggccacattgg |
| | | gactgagacacggcccaaactcctacgggaggcagcagtggggaatattgcacaatgggggaaaccctgatgcagcgacg |
| | | ccgcgtgagtgaagaagtatctcggtatgtaaagctctatcagcagggaagaaaatgacggtacctgactaagaagccccgg |
| | | ctaactacgtgccagcagccgcggtaatacgtagggggcaagcgttatccggaattactgggtgtaaagggtgcgtaggtggt |
| | | atggcaagtcagaagtgaaaacccagggcttaactctgggactgcttttgaaactgtcagactggagtgcaggagaggtaagc |
| | | ggaattcctagtgtagcggtgaaatgcgtagatattaggaggaacatcagtggcgaaggcggcttactggactgaaactgaca |
| | | ctgaggcacgaaagcgtggggagcaaacaggattagataccctggtagtccacgccgtaaacgatgaatactaggtgtcgg |
| | | ggccgtagaggcttcggtgccgcagccaacgcagtaagtattccacctggggagtacgttcgcaagaatgaaactcaaagga |
| | | attgacggggacccgcacaagcggtggagcatgtggtttaattcgaagcaacgcgaagaaccttacctggtcttgacatccttc |
| | | tgaccggtccttaaccggacctttccttcgggacaggagagacaggtggtgcatggttgtcgtcagctcgtgtcgtgagatgttg |
| | | ggttaagtcccgcaacgagcgcaacccctatctttagtagccagcatataaggtgggcactctagagagactgccagggataa |
| | | cctggaggaaggtggggacgacgtcaaatcatcatgccccttatgaccagggctacacacgtgctacaatggcgtaaacaga |
| | | gggaagcagcctcgtgagagtgagcaaatcccaaaaataacgtctcagttcggattgtagtctgcaactcgactacatgaagct |
| | | ggaatcgctagtaatcgcgaatcagaatgtcgcggtgaatacgttcccgggtcttgtacacaccgcccgtcacaccatgggag |
| | | tcagtaacgcccgaagtcagtgacccaaccgtaaggagggagctgccgaaggcgggaccgataactggggtgaagtcgta |
| | | acaaggtagccgtatcggaaggtgcggctggatcacctccttt |
| Lachnospiraceae_ | SEQ ID | aacgagagtttgatcctggctcaggatgaacgctggcggcgtgcctaacacatgcaagtcgagcgaagcactttgcttagattc |
| bacterium_ | NO: 135 | ttcggatgaagaggattgtgactgagcggcggacgggtgagtaacgcgtgggtaacctgcctcatacagggggataacagtt |
| 8_1_57 | | agaaatgactgctaataccgcataagaccacagcaccgcatggtgcgggggtaaaaactccggtggtatgagatggacccgc |
| FAA | | gtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatcccg |
| | | ctgaccggacggtaatgcgtccttcctttcggggcagcggagacaggtggtgcatggttgtcgtcagctcgtgtcgtgagatgt |
| | | tgggttaagtcccgcaacgagcgcaacccctatctttagtagccagcggccaggccgggcactctagagagactgccgggg |
| | | ataacccggaggaaggtggggatgacgtcaaatcatcatgccccttatgagcagggctacacacgtgctacaatggcgtaaa |
| | | caaagggaagcgagaccgcgaggtggagcaaatcccaaaaataacgtctcagttcggattgtagtctgcaactcgactacatg |
| | | aagctggaatcgctagtaatcgcgaatcagaatgtcgcggtgaatacgttcccgggtcttgtacacaccgcccgtcacaccatg |
| | | ggagtcagtaacgcccgaagtcagtgacccaaccgtaaggagggagctgccgaaggcgggaccgataactggggtgaagt |
| | | cgtaacaaggtagccgtatcggaaggtgcggctggatcacctccttt |
| Streptococcus_ | SEQ ID | aatgagagtttgatcctggctcaggatgaacgctggcggcgtgcctaatacatgcaagtagaacgctgaaggaaggagcttgc |
| australis | NO: 136 | tctttctggatgagttgcgaacgggtgagtaacgcgtaggtaacctgcctggtagcgggggataactattggaaacgatagcta |
| | | ataccgcataacagtagatgttgcatgatatctgcttgaaaggggcaattgctccactaccagatggacctgcgttgtattagcta |
| | | gttggtgaggtaacggctcaccaaggcgacgatacatagccgacctgagagggtgatcggccacactgggactgagacacg |
| | | gcccagactcctacgggaggcagcagtagggaatcttcggcaatggacggaagtctgaccgagcaacgccgcgtgagtga |
| | | agaaggttttcggatcgtaaagctctgttgtaagagaagaacgagtgtgagagtggaaagttcacactgtgacggtatcttacca |
| | | gaaagggacggctaactacgtgccagcagccgcggtaatacgtaggtcccgagcgttatccggatttattgggcgtaaagcg |
| | | agcgcaggcggttagataagtctgaagttaaaggctgtggcttaaccatagtacgctttggaaactgtttaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcaaacaggattagataccctggtagtccacgccgtaaacgatgagtgct |
| | | aggtgttgggtcctttccgggactcagtgccgtagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatccctctgatcgctctagagatagagttttccttcgggacagaggtgacaggtggtgcatggttgtcgtcagctcgtgtcg |
| | | tgagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggctgg |
| | | tacaacgagtcgcaagtcggtgacggcaagctaatctcttaaagccagtctcagttcggattgtaggctgcaactcgcctacat |
| | | gaagtcggaatcgctagtaatcgcggatcagcacgccgcggtgaatacgttcccgggccttgtacacaccgcccgtcacacc |
| | | acgagagtttgtaacacccgaagtcggtgaggtaaccttttaggagccagccgcctaaggtgggatagatgattggggtgaag |
| | | tcgtaacaaggtagccgtatcggaaggtgcggctggatcacctcctt |
| Lachnospiraceae_ | SEQ ID | aacgagagtttgatcctggctcaggatgaacgctggcggcgtgcttaacacatgcaagtcgaacgaagcacttgaatggaatt |
| bacterium_ | NO: 137 | cttcggaaggaagctcaagtgactgagtggcggacgggtgagtaacgcgtgggtaacctgcctcatacagggggataacag |
| 4_1_37 | | ttagaaatgactgctaataccgcataagcacacgtgatcgcatgatcgagtgtgaaaaactccggtggtatgagatggacccgc |
| FAA | | gtctgattagctagttggtggggtaacggcccaccaaggcgacgatcagtagccggcctgagagggtgaacggccacattg |
| | | ggactgagacacggcccaaactcctacgggaggcagcagtggggaatattgcacaatgggggaaaccctgatgcagcgac |
| | | gccgcgtgaaggatgaagtatttcggtatgtaaacttctatcagcagggaagaaaatgacggtacctgactaagaagccccgg |
| | | ctaactacgtgccagcagccgcggtaatacgtagggggcaagcgttatccggatttactgggtgtaaagggagcgtagacgg |
| | | cagtgcaagtctgaagtgaaagcccggggctcaaccccgggactgctttggaaactgtgcagctagagtgtcggagaggca |
| | | agcggaattcctagtgtagcggtgaaatgcgtagatattaggaggaacaccagtggcgaaggcggcttgctggacgatgact |
| | | gacgttgaggctcgaaagcgtggggagcaaacaggattagataccctggtagtccacgccgtaaacgatgactactaggtgt |
| | | cggggagcaaagctcttcggtgccgcagccaacgcaataagtagtccacctggggagtacgttcgcaagaatgaaactcaaa |
| | | ggaattgacggggacccgcacaagcggtggagcatgtggtttaattcgaagcaacgcgaagaaccttacctgctcttgacatc |
| | | cctctgaccggcaagtaatgttgcctttccttcgggacagaggagacaggtggtgcatggttgtcgtcagacgtgtcgtgagat |
| | | gttgggttaagtcccgcaacgagcgcaacccctatcttcagtagccagcggtaaggccgggcactctggagagactgccagg |
| | | gataacctggaggaaggtggggatgacgtcaaatcatcatgccccttatgagcagggctacacacgtgctacaatggcgtaaa |
| | | caaagggaagcagagtcgtgaggccgagcaaatcccaaaaataacgtctcagttcggattgtagtctgcaactcgactacatg |
| | | aagctggaatcgctagtaatcgcgaatcagaatgtcgcggtgaatacgttcccgggtcttgtacacaccgcccgtcacaccatg |
| | | ggagtcagtaacgcccgaagtcagtgacccaaccgcaaggagggagctgccgaaggtgggaccgataactggggtgaagt |
| | | cgtaacaaggtagccgtatcggaaggtgcggctggatcacctccttt |
| Streptococcus_ | SEQ ID | aatgagagtttgatcctggctcaggacgaacgctggcggcgtgcctaatacatgcaagtagaacgctgaagagaggagcttg |
| sanguinis | NO: 138 | ctcttcttggatgagttgcgaacgggtgagtaacgcgtaggtaacctgcctggtagcgggggataactattggaaacgatagct |
| | | aataccgcatgatattaattatcgcatgataattaattgaaagatgcaattgcatcactaccagatggacctgcgttgtattagctag |
| | | ttggtgaggtaacggctcaccaaggcgacgatacatagccgacctgagagggtgatcggccacactgggactgagacacgg |
| | | cccagactcctacgggaggcagcagtagggaatcttcggcaatggggggaaccctgaccgagcaacgccgcgtgagtgaa |
| | | gaaggttttcggatcgtaaagctctgttgtaagagaagaacgggtgtgagagtggaaagttcacactgtgacggtatcttacca |
| | | gaaagggacggctaactacgtgccagcagccgcggtaatacgtaggtcccgagcgttgtccggatttattgggcgtaaagcg |
| | | agcgcaggcggttagataagtctgaagttaaaggctgtggcttaaccatagtatgctttggaaactgtttaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcaaacaggattagataccctggtagtccacgccgtaaacgatgagtgct |
| | | aggtgttaggccctttccggggcttagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatccctctgaccgctctagagatagagttttccttcgggacagaggtgacaggtggtgcatggttgtcgtcagctcgtgtc |
| | | gtgagatgttgggttaagtcccgcaacgagcgcaacccctattgttagttgccatcattgagttgggcactctagcgagactgcc |
| | | ggtaataaaccggaggaaggtggggatgacgtcaaatcatcatgccccttatgacctgggctacacacgtgctacaatggctg |
| | | gtacaacgagtcgcaagccggtgacggcaagctaatctctgaaagccagtctcagttcggattgtaggctgcaactcgcctac |
| | | atgaagtcggaatcgctagtaatcgcggatcagcacgccgcggtgaatacgttcccgggccttgtacacaccgcccgtcacac |
| | | cacgagagtttgtaacacccgaagtcggtgaggtaaccgtaaggagccagccgcctaaggtgggatagatgattggggtgaa |
| | | gtcgtaacaaggtagccgtatcggaaggtgcggctggatcacctcctt |
| Lachnospiraceae_ | SEQ ID | aacgagagtttgatcctggctcaggatgaacgctggcggcgtgcctaacacatgcaagtcgagcgaagcactttgcttagattc |
| bacterium_ | NO: 139 | ttcggatgaagaggattgtgactgagcggcggacgggtgagtaacgcgtgggtaacctgcctcatacagggggataacagtt |
| 3_1_46 | | agaaatgactgctaataccgcataagaccacagcaccgcatggtgcgggggtaaaaactccggtggtatgagatggacccgc |
| FAA | | gtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatcccg |
| | | ctgaccggacggtaatgcgtccttcccttcggggcagcggagacaggtggtgcatggttgtcgtcagctcgtgtcgtgagatgt |
| | | tgggttaagtcccgcaacgagcgcaacccctatctttagtagccagcggccaggccgggcactctagagagactgccgggg |
| | | ataacccggaggaaggtggggatgacgtcaaatcatcatgccccttatgagcagggctacacacgtgctacaatggcgtaaa |
| | | caaagggaagcgagaccgcgaggtggagcaaatcccaaaaataacgtctcagttcggattgtagtctgcaactcgactacatg |
| | | aagctggaatcgctagtaatcgcgaatcagaatgtcgcggtgaatacgttcccgggtcttgtacacaccgcccgtcacaccatg |
| | | ggagtcagtaacgcccgaagtcagtgacccaaccgtaaggagggagctgccgaaggcgggaccgataactggggtgaagt |
| | | cgtaacaaggtagccgtatcggaaggtgcggctggatcacctccttt |
| Lachnospiraceae_ | SEQ ID | tcagagagtttgatcctggctcaggatgaacgctggcggcgtgcttaacacatgcaagtcgagcgaagcacttatcattgactct |
| bacterium_ | NO: 140 | tcggaagatttgatatttgactgagcggcggacgggtgagtaacgcgtgggtaacctgcctcatacaggggaataacagttag |
| 6_1_63 | | aaatggctgctaatgccgcataagcgcacaggaccgcatggtctggtgtgaaaaactgaggtggtatgagatggacccgcgt |
| FAA | | ctgattaggtagttggtggggtaacggcctaccaagccgacgatcagtagccggcctgagagggtgaacggccacattggg |
| | | actgagacacggcccagactcctacgggaggcagcagtggggaatattgcacaatgggggaaaccctgatgcagcgacgc |
| | | cgcgtgaaggaagaagtatctcggtatgtaaacttctatcagcagggaagaaaatgacggtacctgactaagaagccccggct |
| | | aactacgtgccagcagccgcggtaatacgtagggggcaagcgttatccggatttactgggtgtaaagggagcgtagacggaa |
| | | gagcaagtctgatgtgaaaggctggggcttaaccccaggactgcattggaaactgtttttctagagtgccggagaggtaagcg |
| | | gaattcctagtgtagcggtgaaatgcgtagatattaggaggaacaccagtggcgaaggcggcttactggacggtaactgacgt |
| | | tgaggctcgaaagcgtggggagcaaacaggattagataccctggtagtccacgccgtaaacgatgaatactaggtgtcgggg |
| | | tgcaaagcagttcggtgccgcagcaaacgcaataagtattccacctggggagtacgttcgcaagaatgaaactcaaaggaatt |
| | | gacggggacccgcacaagcggtggagcatgtggtttaattcgaagcaacgcgaagaaccttaccaagtcttgacatctgcctg |
| | | accgttccttaaccggagctttccttcgggacaggcaagacaggtggtgcatggttgtcgtcagctcgtgtcgtgagatgttggg |
| | | ttaagtcccgcaacgagcgcaacccctatccttagtagccagcagtccggctgggcactctagggagactgccggggataac |
| | | ccggaggaaggcggggacgacgtcaaatcatcatgccccttatgatttgggctacacacgtgctacaatggcgtaaacaaag |
| | | ggaagcgaagcggtgacgcttagcaaatctcaaaaataacgtcccagttcggactgcagtctgcaactcgactgcacgaagct |
| | | ggaatcgctagtaatcgcgaatcagaatgtcgcggtgaatacgttcccgggtcttgtacacaccgcccgtcacaccatgggag |
| | | tcagtaacgcccgaagtcagtgacccaaccttatggagggagctgccgaaggcgggaccgataactggggtgaagtcgtaa |
| | | caaggtagccgtatcggaaggtgcggctggatcacctccttt |
| Lachnospiraccae | SEQ ID | aacgagagtttgatcctggctcaggatgaacgctggcggcgtgcttaacacatgcaagtcgaacgaagcacttgaatggaatt |
| bacterium_ | NO: 141 | cttcggaaggaagctcaagtgactgagtggcggacgggtgagtaacgcgtgggtaacctgcctcatacagggggataacag |
| 9_1_43 | | ttagaaatgactgctaataccgcataagcacacgtgatcgcatgatcgagtgtgaaaaactccggtggtatgagatggacccgc |
| BFAA | | gtctgattagctagttggtggggtaatggcccaccaaggcgacgatcagtagccggcctgagagggtgaacggccacattgg |
| | | gactgagacacggcccaaactcctacgggaggcagcagtggggaatattgcacaatgggggaaaccctgatgcagcgacg |
| | | ccgcgtgaaggatgaagtatttcggtatgtaaacttctatcagcagggaagaaaatgacggtacctgactaagaagccccggc |
| | | taactacgtgccagcagccgcggtaatacgtagggggcaagcgttatccggatttactgggtgtaaagggagcgtagacggc |
| | | agtgcaagtctgaagtgaaagcccggggctcaaccccgggactgctttggaaactgtgcagctagagtgtcggagaggcaa |
| | | gcggaattcctagtgtagcggtgaaatgcgtagatattaggaggaacaccagtggcgaaggcggcttgctggacgatgactg |
| | | acgttgaggctcgaaagcgtggggagcaaacaggattagataccctggtagtccacgccgtaaacgatgactactaggtgtc |
| | | ggggagcaaagctcttcggtgccgcagccaacgcaataagtagtccacctggggagtacgttcgcaagaatgaaactcaaag |
| | | gaattgacggggacccgcacaagcggtggagcatgtggtttaattcgaagcaacgcgaagaaccttacctgctcttgacatcc |
| | | ctctgaccgctctttaatcggagctttccttcgggacagaggagacaggtggtgcatggttgtcgtcagctcgtgtcgtgagatg |
| | | ttgggttaagtcccgcaacgagcgcaacccctatcttcagtagccagcggtaaggccgggcactctggagagactgccaggg |
| | | ataacctggaggaaggtggggatgacgtcaaatcatcatgccccttatgagcagggctacacacgtgctacaatggcgtaaac |
| | | aaagggaagcagagtcgtgaggccgagcaaatcccaaaaataacgtctcagttcggattgtagtctgcaactcgactacatga |
| | | agctggaatcgctagtaatcgcgaatcagaatgtcgcggtgaatacgttcccgggtcttgtacacaccgcccgtcacaccatgg |
| | | gagtcagtaacgcccgaagtcagtgacccaaccgcaaggagggagctgccgaaggtgggaccgataactggggtgaagtc |
| | | gtaacaaggtagccgtatcggaaggtgcggctggatcacctccttt |
| Ruminococcus_ | SEQ ID | atatcaaagattttatcgcttgaagatggactcgcgtccgattagttagttggtgaggtaacggctcaccaagaccgcgatcggt |
| bromii | NO: 142 | agccggactgagaggttgaacggccacattgggactgagacacggcccagactcctacgggaggcagcagtgggggatat |
| | | tgcgcaatgggggcaaccctgacgcagcaacgccgcgtgaaggatgaaggttttcggattgtaaacttcttttattaaggacga |
| | | aacttgacggtacttaatgaataagctccggctaactacgtgccagcagccgcggtaatacgtagggagcaagcgttgtccgg |
| | | atttactgggtgtaaagggtgcgtaggcggctttgcaagtcagatgtgaaatctatgggctcaacccataaactgcatttgaaact |
| | | gtagagcttgagtgaagtagaggcaggcggaattccccgtgtagcggtgaaatgcgtagagatggggaggaacaccagtgg |
| | | cgaaggcggcctgctgggctttaactgacgctgaggcacgaaagcgtgggtagcaaacaggattagataccctggtagtcca |
| | | cgctgtaaacgatgattactaggtgtggggggtctgaccccttccgtgccggagttaacacaataagtaatccacctggggagt |
| | | acggccgcaaggttgaaactcaaaggaattgacgggggcccgcacaagcagtggagtatgtggtttaattcgaagcaacgc |
| | | gaagaaccttaccaggtcttgacatccaactaacgaagtagagatacattaggtgcccttcggggaaagttgagacaggtggt |
| | | gcatggttgtcgtcagctcgtgtcgtgagatgttgggttaagtcccgcaacgagcgcaacccttgctattagttgctacgcaaga |
| | | gcactctaataggactgccgttgacaaaacggaggaaggtggggacgacgtcaaatcatcatgccccttatgacctgggctac |
| | | acacgtactacaatggatgttaacagagggaagcaagacagcgatgtggagcaaacccctaaaaacattctcagttcagattg |
| | | caggctgcaacccgcctgcatgaagatggaattgctagtaatcgcggatcagcatgccgcggtgaatacgttcccgggccttg |
| | | tacacaccgcccgtcacaccatgggagccggtaatacccgaagtcagtagtccaacctcgtgaggacgctgccgaaggtag |
| | | gattggcgactggggtgaagtcgtaacaaggtagccgtatcggaaggtgcggctggatcacctccttt |
| Coprococcus_ | SEQ ID | atgagagtttgatcctggctcaggatgaacgctggcggcgtgcttaacacatgcaagtcgaacggacgatgaagagcttgcttt |
| catus | NO: 143 | tcaaagttagtggcggacgggtgagtaacgcgtgggtaacctgcctcatacagggggatagcagctggaaacggctgataaa |
| | | accgcataagcgcacagcatcgcatgatgcagtgtgaaaaactccggtggtatgagatggacccgcgtctgattagctggttg |
| | | gtgaggtaacggcccaccaaggcgacgatcagtagccggcctgagagggtgaccggccacattgggactgagacacggc |
| | | ccaaactcctacgggaggcagcagtggggaatattgcacaatgggggaaaccctgatgcagcgacgccgcgtgaaggaag |
| | | aagtatctcggtatgtaaacttctatcagcagggaagataatgacggtacctgactaagaagccccggctaactacgtgccagc |
| | | agccgcggtaatacgtagggggcaagcgttatccggatttactgggtgtaaagggagcgtaggcggcggagcaagtcagaa |
| | | gtgaaagcccggggctcaaccccgggacggcttttgaaactgccctgcttgatttcaggagaggtaagcggaattcctagtgt |
| | | agcggtgaaatgcgtagatattaggaggaacaccagtggcgaaggcggcttactggactgacaatgacgctgaggctcgaa |
| | | agcgtggggagcaaacaggattagataccctggtagtccacgccgtaaacgatgaatactaggtgtcgggggctcataagag |
| | | ctttcggtgccgcagcaaacgcaataagtattccacctggggagtacgttcgcaagaatgaaactcaaaggaattgacgggga |
| | | cccgcacaagcggtggagcatgtggtttaattcgaagcaacgcgaagaaccttaccaggccttgacatcccnnn |
| Dysgonomonas_ | SEQ ID | agaagagtttgatcctggctcaggatgaacgctagcgataggcctaacacatgcaagtcgaggggcagcatagagtagcaat |
| mossii | NO: 144 | actttgatggcgaccggcgcacgggtgagtaacacgtatgcaacctacctacatcaggggaataacccggagaaattcggac |
| | | taataccgcataatacagggatgccgcatggcaatatttgttaaagatttatcggatgtagatgggcatgcgttccattagctggtt |
| | | ggtaaggtaacggcttaccaaggcaacgatggataggggaactgagaggtttgtcccccacactggaactgagacacggac |
| | | cagactcctacgggaggcagcagtgaggaatattggtcaatggacgagagtctgaaccagccaaatcgcgtgaaggaagac |
| | | tgccctacgggttgtaaacttcttttgtacaggaataaaagacattacgtgtagtgtattgcatgtactgtacgaataagcatcggct |
| | | aactccgtgccagcagccgcggtaatacggaggatgcgagcgttatccggatttattgggtttaaagggtgcgcaggcgggat |
| | | tttaagtcagcggtgaaattttcaggctcaacctgaacactgccgttgaaactgggattcttgagtatggatgaagtaggcggaat |
| | | tcgttgtgtagcggtgacatgcttagatataacgaggaactccgattgcgtaggcagcttactaagccataactgacgctcaagc |
| | | acgaaagcgtggggatcaaacaggattagataccctggtagtccacgccgtaaacgatgattactagttgtttgcgatataccgt |
| | | aagtgactaagcgaaagcgataagtaatccacctggggagtacgccggcaacggtgaaactcaaaggaattgacgggggc |
| | | ccgcacaagcggaggaacatgtggtttaattcgatgatacgcgaggaaccttacccgggcttgaaatgtacctgaccgtccttg |
| | | aaaggggacttctagcaatagcaggtatgtaggtgctgcatggttgtcgtcagctcgtgccgtgaggtgtcggcttaagtgccat |
| | | aacgagcgcaacccacattattagttactaacaggttaagctgaggactctaataagactgccgtcgtaagatgcgaggaaggt |
| | | gtggatgacgtcaaatcagcacggcccttacgtccggggcgacacacgtgttacaatgggaggtacaaagggctgctacctg |
| | | gtgacaggatgctaatccccaaagcctctcccagttcggatcggagtctgcaactcgactccgtgaagctggattcgctagtaa |
| | | tcgcgcatcagccatggcgcggtgaatacgttcccgggccttgtacacaccgcccgtcaagccatgggagccgggggtatct |
| | | gaagtgcgtaaccgtaaggagcgtcctagggtaaaactggtgactggggctaagtcgtaacaaggtagccgtaccggaaggt |
| | | gcggctggaacacctcctt |
| Lachnospiraceae_ | SEQ ID | aacgagagtttgatcctggctcaggatgaacgctggcggcgtgcttaacacatgcaagtcgaacgaagcactttaccggatttc |
| bacterium_ | NO: 145 | ttcgggatgaaagttttgtgactgagtggcggacgggtgagtaacgcgtgggtaacctgcctcatacagggggataacagtta |
| 1_4_56 | | gaaatgactgctaataccgcataagaccacaggattgcatgatccggtggtaaaaactccggtggtatgagatggacccgcgt |
| FAA | | ctgattagctagttggtaaggtaacggcttaccaaggcgacgatcagtagccgacctgagagggtgaccggccacattggga |
| | | ctgagacacggcccaaactcctacgggaggcagcagtggggaatattgcacaatgggggaaaccctgatgcagcgacgcc |
| | | gcgtgagcgatgaagtatttcggtatgtaaagctctatcagcagggaagaaaatgacggtacctgactaagaagcaccggcta |
| | | aatacgtgccagcagccgcggtaatacgtatggtgcaagcgttatccggatttactgggtgtaaagggagcgtagacggaga |
| | | ggcaagtctgatgtgaaaacccggggctcaaccccgggactgcattggaaactgtttttctagagtgtcggagaggtaagtgg |
| | | aattcctagtgtagcggtgaaatgcgtagatattaggaggaacaccagtggcgaaggcggcttactggacgatgactgacgtt |
| | | gaggctcgaaagcgtggggagcaaacaggattagataccctggtagtccacgccgtaaacgatgactgctaggtgtcggga |
| | | ggcaaagcctttcggtgccgcagcaaacgcaataagcagtccacctggggagtacgttcgcaagaatgaaactcaaaggaat |
| | | tgacggggacccgcacaagcggtggagcatgtggtttaattcgaagcaacgcgaagaaccttacctgcccttgacatccggct |
| | | gaccggcgagtaatgtcgcctttccttcgggacagccgagacaggtggtgcatggttgtcgtcagctcgtgtcgtgagatgttg |
| | | ggttaagtcccgcaacgagcgcaacccttatctttagtagccagcatttcggatgggcactctagagagactgccagggataac |
| | | ctggaggaaggtggggatgacgtcaaatcatcatgccccttatgggcagggctacacacgtgctacaatggcgtaaacaaag |
| | | ggaggcaagcctgcgagggtgagcaaatcccaaaaataacgtctcagttcggattgtagtctgcaactcgactacatgaagct |
| | | ggaatcgctagtaatcgcgaatcagaatgtcgcggtgaatacgttcccgggtcttgtacacaccgcccgtcacaccatgggag |
| | | ttggtaacgcccgaagtcagtgacccaaccgtaaggagggagctgccgaaggtgggaccgataactggggtgaagtcgtaa |
| | | caaggtagccgtatcggaaggtgcggctggatcacctccttt |
| Lachnospiraceae_ | SEQ ID | agagagtttgatcctggctcaggatgaacgctggcggcgtgcctaacacatgcaagtcgaacgaagttacacggaggaagttt |
| bacterium_ | NO: 146 | tcggatggaatcggtataacttagtggcggacgggtgagtaacgcgtgggaaacctgccctgtaccgggggataacacttag |
| 3_1_57 | | aaataggtgctaataccgcataagcgcacggaaccgcatggttccgtgtgaaaaactccggtggtacaggatggtcccgcgtc |
| FAA_CT1 | | tgattagccagttggcagggtaacggcctaccaaagcgacgatcagtagccggcctgagagggtgaacggccacattggga |
| | | ctgagacacggcccaaactcctacgggaggcagcagtggggaatattgcacaatgggggaaaccctgatgcagcgacgcc |
| | | gcgtgagtgaagaagtatttcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgtagacggcat |
| | | ggcaagccagatgtgaaaacccagggctcaaccttgggattgcatttggaactgccaggctggagtgcaggagaggtaagc |
| | | ggaattcctagtgtagcggtgaaatgcgtagatattaggaggaacaccagtggcgaaggcggcttactggactgtaactgacg |
| | | ttgaggctcgaaagcgtggggagcaaacaggattagataccctggtagtccacgcggtaaacgatgattgctaggtgtaggtg |
| | | ggtatggacccatcggtgccgcagctaacgcaataagcaatccacctggggagtacgttcgcaagaatgaaactcaaaggaa |
| | | ttgacggggacccgcacaagcggtggagcatgtggtttaattcgaagcaacgcgaagaaccttaccaagtcttgacatccccc |
| | | tgaatatcttgtaaaaaagagatgccttcgggacaggggagacaggtggtgcatggttgtcgtcagctcgtgtcgtgagatgtt |
| | | gggttaagtcccgcaacgagcgcaacccttattatagtagccagcaggtaaggctgggcactctaaggagactgccgggga |
| | | taacccggaggaaggcggggatgacgtcaaatcatcatgccccttatgatttgggctacacacgtgctacaatggcgtaaaca |
| | | aagggaagcgagacagtgatgtggagcaaatcccagaaataacgtctcagttcggattgtagtctgcaactcgactacatgaa |
| | | gctggaatcgctagtaatcgcgaatcagcatgtcgcggtgaatacgttcccgggtcttgtacacaccgcccgtcacaccatgg |
| | | gagttggaaatgcccgaagtctgtgacctaaccgaaagggaggagcagccgaaggcaggtctgataactggggtgaagtcg |
| | | taacaaggtagccgtatcggaaggtgcggctggatcacctccttt |
| Lachnospiraceae_ | SEQ ID | agagagtttgatcctggctcaggatgaacgctggcggcgtgcctaacacatgcaagtcgaacgaagttatatcgaggaagtttt |
| bacterium_ | NO: 147 | cggatggaatcagtataacttagtggcggacgggtgagtaacgcgtgggaaacctgccctgtaccgggggataacacttaga |
| 3_1_57 | | aataggtgctaataccgcataagcgcacagcttcacatgaagcagtgtgaaaaactccggtggtacaggatggtcccgcgtct |
| FAA_CT1 | | gattagccagttggcagggtaacggcctaccaaagcgacgatcagtagccggcctgagagggtgaacggccacattgggac |
| | | tgagacacggcccaaactcctacgggaggcagcagtggggaatattgcacaatgggggaaaccctgatgcagcgacgccg |
| | | cgtgagtgaagaagtatttcggtatgtaaagctctatcagcagggaagaaaatgacggtacctgactaagaagccccggctaa |
| | | ctacgtgccagcagccgcggtaatacgtagggggcaagcgttatccggatttactgggtgtaaagggagcgtagacggcatg |
| | | gcaagccagatgtgaaaacccagggctcaaccttgggattgcatttggaactgtcaggctggagtgcaggagaggtaagcgg |
| | | aattcctagtgtagcggtgaaatgcgtagatattaggaggaacaccagtggcgaaggcggcttactggactgtaactgacgttg |
| | | aggctcgaaagcgtggggagcaaacaggattagataccctggtagtccacgcggtaaacgatgattgctaggtgtaggtggg |
| | | tatggacccatcggtgccgcagctaacgcaataagcaatccacctggggagtacgttcgcaagaatgaaactcaaaggaattg |
| | | acggggacccgcacaagcggtggagcatgtggtttaattcgaagcaacgcgaagaaccttaccaagtcttgacatccccctga |
| | | ctctcctgtaaaagagtgaggccttcgggacattggagacaggtggtgcatggttgtcgtcagctcgtgtcgtgagatgttgggt |
| | | taagtcccgcaacgagcgcaacccttattcttagtagccagcaggtaaagctgggcactctaaggagactgccggggataacc |
| | | cggaggaaggcggggatgacgtcaaatcatcatgccccttatgatttgggctacacacgtgctacaatggcgtaaacaaagg |
| | | gaagcgagacagtgatgtggagcaaatcccagaaataacgtctcagttcggattgtagtctgcaactcgactacatgaagctg |
| | | gaatcgctagtaatcgcgaatcagcatgtcgcggtgaatacgttcccgggtcttgtacacaccgcccgtcacaccatgggagtt |
| | | ggaaatgcccgaagtctgtgacctaaccgaaagggaggagcagccgaaggcaggtctgataactggggtgaagtcgtaaca |
| | | aggtagccgtatcggaaggtgcggctggatcacctccttt |
| Lachnospiraceae_ | SEQ ID | agagagtttgatcctggctcaggatgaacgctggcggcgtgcctaacacatgcaagtcgaacgaagttacacagaggaagttt |
| bacterium_ | NO: 148 | tcggatggaatcggtataacttagtggcggacgggtgagtaacgcgtgggaaacctgccctgtaccgggggataacacttag |
| 3_1_57 | | aaataggtgctaataccgcataagcgcacggattcacatgaagcagtgtgaaaaactccggtggtacaggatggtcccgcgtc |
| FAA_CT1 | | tgattagccagttggcagggtaacggcctaccaaagcgacgatcagtagccggcctgagagggtgaacggccacattggga |
| | | ctgagacacggcccaaactcctacgggaggcagcagtggggaatattgcacaatgggggaaaccctgatgcagcgacgcc |
| | | gcgtgagtgaagaagtatttcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgtagacggcat |
| | | ggcaagccagatgtgaaaacccagggctcaaccttgggattgcatttggaactgtcaggctggagtgcaggagaggtaagcg |
| | | gaattcctagtgtagcggtgaaatgcgtagatattaggaggaacaccagtggcgaaggcggcttactggactgtaactgacgtt |
| | | gaggctcgaaagcgtggggagcaaacaggattagataccctggtagtccacgcggtaaacgatgattgctaggtgtaggtgg |
| | | gtatggacccatcggtgccgcagctaacgcaataagcaatccacctggggagtacgttcgcaagaatgaaactcaaaggaatt |
| | | gacggggacccgcacaagcggtggagcatgtggtttaattcgaagcaacgcgaagaaccttaccaagtcttgacatcccaat |
| | | gacgcacctgtaaagaggtgttcccttcggggcattggagacaggtggtgcatggttgtcgtcagctcgtgtcgtgagatgttg |
| | | ggttaagtcccgcaacgagcgcaacccttattcttagtagccagcaggtaaagctgggcactctaaggagactgccggggata |
| | | acccggaggaaggcggggatgacgtcaaatcatcatgccccttatgatttgggctacacacgtgctacaatggcgtaaacaaa |
| | | gggaagcgagacagtgatgtggagcaaatcccagaaataacgtctcagttcggattgtagtctgcaactcgactacatgaagc |
| | | tggaatcgctagtaatcgcgaatcagcatgtcgcggtgaatacgttcccgggtcttgtacacaccgcccgtcacaccatggga |
| | | gttggaaatgcccgaagtctgtgacctaaccgaaagggaggagcagccgaaggcaggtctgataactggggtgaagtcgta |
| | | acaaggtagccgtatcggaaggtgcggctggatcacctccttt |
| Lachnospiraceae_ | SEQ ID | aacgagagtttgatcctggctcaggatgaacgctggcggcgtgcctaacacatgcaagtcgaacgaagcgcctggccccga |
| bacterium_ | NO: 149 | cttcttcggaacgaggagccttgcgactgagtggcggacgggtgagtaacgcgtgggcaacctgccttgcactgggggataa |
| 5_1_57 | | cagccagaaatggctgctaataccgcataagaccgaagcgccgcatggcgcggcggccaaagccccggcggtgcaagat |
| FAA | | gggcccgcgtctgattaggtagttggcggggtaacggcccaccaagccgacgatcagtagccgacctgagagggtgaccg |
| | | gccacattgggactgagacacggcccagactcctacgggaggcagcagtggggaatattgcacaatgggggaaaccctgat |
| | | gcagcgacgccgcgtgaaggatgaagtatttcggtatgtaaacttctatcagcagggaagaagatgacggtacctgactaaga |
| | | agccccggctaactacgtgccagcagccgcggtaatacgtagggggcaagcgttatccggatttactgggtgtaaagggagc |
| | | gtagacggcgatgcaagccagatgtgaaagcccggggctcaaccccgggactgcatttggaactgcgtggctggagtgtcg |
| | | gagaggcaggcggaattcctagtgtagcggtgaaatgcgtagatattaggaggaacaccagtggcgaaggcggcctgctgg |
| | | acgatgactgacgttgaggctcgaaagcgtggggagcaaacaggattagataccctggtagtccacgccgtaaacgatgact |
| | | actaggtgtcgggtggcaaggccattcggtgccgcagcaaacgcaataagtagtccacctggggagtacgttcgcaagaatg |
| | | aaactcaaaggaattgacggggacccgcacaagcggtggagcatgtggtttaattcgaagcaacgcgaagaaccttacctga |
| | | tcttgacatcccgatgccaaagcgcgtaacgcgctctttcttcggaacatcggtgacaggtggtgcatggttgtcgtcagctcgt |
| | | gtcgtgagatgttgggttaagtcccgcaacgagcgcaacccctatcttcagtagccagcattatggatgggcactctggagaga |
| | | ctgccagggagaacctggaggaaggtggggatgacgtcaaatcatcatgccccttatgaccagggctacacacgtgctacaa |
| | | tggcgtaaacaaagggaggcgaacccgcgagggtgggcaaatcccaaaaataacgtctcagttcggattgtagtctgcaact |
| | | cgactacatgaagctggaatcgctagtaatcgcgaatcagaatgtcgcggtgaatacgttcccgggtatgtacacaccgcccg |
| | | tcacaccatgggagtcagtaacgcccgaagccggtgacccaacccgtaagggagggagccgtcgaaggtgggaccgataa |
| | | ctggggtgaagtcgtaacaaggtagccgtatcggaaggtgcggctggatcacctccttt |
| Lachnospiraceae_ | SEQ ID | aacgagagtttgatcctggctcaggatgaacgctggcggcgtgcctaacacatgcaagtcgagcgaagcactttgatagattc |
| bacterium_ | NO: 150 | ttcggatgaagaggattgtgactgagcggcggacgggtgagtaacgcgtgggtaacctgcctcatacagggggataacagtt |
| 1_1_57 | | agaaatgactgctaataccgcataagaccacagcaccgcatggtgcgggggtaaaaactccggtggtatgagatggacccgc |
| FAA | | gtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcagggaagaaaatgacggtacctgactaagaagcaccgg |
| | | ctaaatacgtgccagcagccgcggtaatacgtatggtgcaagcgttatccggatttactgggtgtaaagggagcgtagacgga |
| | | tgggcaagtctgatgtgaaaacccggggctcaaccccgggactgcattggaaactgttcatctagagtgctggagaggtaagt |
| | | ggaattcctagtgtagcggtgaaatgcgtagatattaggaggaacaccagtggcgaaggcggcttactggacagtaactgac |
| | | gttgaggctcgaaagcgtggggagcaaacaggattagataccctggtagtccacgccgtaaacgatgactactaggtgtcgg |
| | | gtggcaaagccattcggtgccgcagcaaacgcaataagtagtccacctggggagtacgttcgcaagaatgaaactcaaagga |
| | | attgacggggacccgcacaagcggtggagcatgtggtttaattcgaagcaacgcgaagaaccttacctgctcttgacatcccg |
| | | ctgaccggacggtaatgcgtccttcccttcggggcagcggagacaggtggtgcatggttgtcgtcagctcgtgtcgtgagatgt |
| | | tgggttaagtcccgcaacgagcgcaacccctatctttagtagccagcggccaggccgggcactctagagagactgccgggg |
| | | ataacccggaggaaggtggggatgacgtcaaatcatcatgccccttatgagcagggctacacacgtgctacaatggcgtaaa |
| | | caaagggaagcgagaccgcgaggtggagcaaatcccaaaaataacgtctcagttcggattgtagtctgcaactcgactacatg |
| | | aagctggaatcgctagtaatcgcgaatcagaatgtcgcggtgaatacgttcccgggtcttgtacacaccgcccgtcacaccatg |
| | | ggagtcagtaacgcccgaagtcagtgacccaaccgtaaggagggagagccgaaggcgggaccgataactggggtgaagt |
| | | cgtaacaaggtagccgtatcggaaggtgcggctggatcacctccttt |
| Lachnospiraceae_ | SEQ ID | aacgagagtttgatcctggctcaggatgaacgctggcggcgtgcttaacacatgcaagtcgagcgaagcaccttgacggattt |
| bacterium_ | NO: 151 | cttcggattgaagccttggtgactgagcggcggacgggtgagtaacgcgtgggtaacctgcctcatacagggggataacagtt |
| 2_1_58 | | ggaaacggctgctaataccgcataagcgcacagtaccgcatggtacggtgtgaaaaactccggtggtatgagatggacccgc |
| FAA | | gtctgattaggtagttggtggggtaacggcctaccaagccgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgatgaagtatttcggtatgtaaagctctatcagcagggaagaaaatgacggtacctgactaagaagccccggc |
| | | taactacgtgccagcagccgcggtaatacgtagggggcaagcgttatccggatttactgggtgtaaagggagcgtagacggc |
| | | atggcaagccagatgtgaaagcccggggctcaaccccgggactgcatttggaactgtcaggctagagtgtcggagaggaaa |
| | | gcggaattcctagtgtagcggtgaaatgcgtagatattaggaggaacaccagtggcgaaggcggctttctggacgatgactga |
| | | cgttgaggctcgaaagcgtggggagcaaacaggattagataccctggtagtccacgccgtaaacgatgaatactaggtgtcg |
| | | ggtggcaaagccattcggtgccgcagcaaacgcaataagtattccacctggggagtacgttcgcaagaatgaaactcaaagg |
| | | aattgacggggacccgcacaagcggtggagcatgtggtttaattcgaagcaacgcgaagaaccttacctggtcttgacatccc |
| | | tctgaccgctctttaatcggagctttccttcgggacagaggagacaggtggtgcatggttgtcgtcagacgtgtcgtgagatgtt |
| | | gggttaagtcccgcaacgagcgcaacccctatctttagtagccagcattttcggatgggcactctagagagactgccagggat |
| | | aacctggaggaaggtggggatgacgtcaaatcatcatgccccttatgaccagggctacacacgtgctacaatggcgtaaacaa |
| | | agggaagcgagcccgcgagggggagcaaatcccaaaaataacgtctcagttcggattgtagtctgcaactcgactacatgaa |
| | | gctggaatcgctagtaatcgcgaatcagaatgtcgcggtgaatacgttcccgggtcttgtacacaccgcccgtcacaccatgg |
| | | gagtcagtaacgcccgaagtcagtgacccaaccgcaaggagggagctgccgaaggtgggaccgataactggggtgaagtc |
| | | gtaacaaggtagccgtatcggaaggtgcggctggatcacctccttt |
| Roseburia_ | SEQ ID | atgagagtttgatcctggctcaggatgaacgctggcggcgtgcttaacacatgcaagtcgaacgaagcactttaattgatttcttc |
| hominis | NO: 152 | ggaatgaagtttttgtgactgagtggcggacgggtgagtaacgcgtgggtaacctgcctcatacagggggataacagttggaa |
| | | acgactgctaataccgcataagcgcacaggattgcatgatccagtgtgaaaaactccggtggtatgagatggacccgcgtctg |
| | | attagccagttggcggggtaacggcccaccaaagcgacgatcagtagccgacctgagagggtgaccggccacattgggact |
| | | gagacacggcccaaactcctacgggaggcagcagtggggaatattgcacaatgggggaaaccctgatgcagcgacgccgc |
| | | gtgagcgaagaagtatttcggtatgtaaagctctatcagcagggaagaagaatgacggtacctgactaagaagcaccggctaa |
| | | atacgtgccagcagccgcggtaatacgtatggtgcaagcgttatccggatttactgggtgtaaagggagcgcaggcggtacg |
| | | gcaagtctgatgtgaaatcccggggctcaaccccggtactgcattggaaactgtcggactagagtgtcggaggggtaagtgg |
| | | aattcctagtgtagcggtgaaatgcgtagatattaggaggaacaccagtggcgaaggcggcttactggacgattactgacgct |
| | | gaggctcgaaagcgtggggagcaaacaggattagataccctggtagtccacgccgtaaacgatgaatactaggtgtcgggg |
| | | agcattgctcttcggtgccgcagcaaacgcaataagtattccacctggggagtacgttcgcaagaatgaaactcaaaggaattg |
| | | acggggacccgcacaagcggtggagcatgtggtttaattcgaagcaacgcgaagaaccttaccaagtcttgacatcccactga |
| | | caaagtatgtaatgtactttctcttcggagcagtggtgacaggtggtgcatggttgtcgtcagctcgtgtcgtgagatgttgggtta |
| | | agtcccgcaacgagcgcaacccctattcttagtagccagcggttcggccgggcactctagggagactgccagggataacctg |
| | | gaggaaggtggggatgacgtcaaatcatcatgccccttatgacttgggctacacacgtgctacaatggcgtaaacaaagggaa |
| | | gcaatcccgcgagggggagcaaatctcaaaaataacgtctcagttcggactgtagtctgcaactcgactacacgaagctggaa |
| | | tcgctagtaatcgcgaatcagaatgtcgcggtgaatacgttcccgggtcttgtacacaccgcccgtcacaccatgggagttggt |
| | | aatgcccgaagtcagtgacccaaccgcaaggagggagctgccgaaggcaggactgataactggggtgaagtcgtaacaag |
| | | gtagccgtatcggaaggtgcggctggatcacctccttt |
| Roseburia_ | SEQ ID | atgagagtttgatcctggctcaggatgaacgctggcggcgtgcttaacacatgcaagtcgaacgaagcactttaattgatttcttc |
| hominis | NO: 153 | ggaatgaagtttttgtgactgagtggcggacgggtgagtaacgcgtgggtaacctgcctcatacagggggataacagttggaa |
| | | acgactgctaataccgcataagcgcacaggattgcatgatccagtgtgaaaaactccggtggtatgagatggacccgcgtctg |
| | | attagccagttggcggggtaacggcccaccaaagcgacgatcagtagccgacctgagagggtgaccggccacattgggact |
| | | gagacacggcccaaactcctacgggaggcagcagtggggaatattgcacaatgggggaaaccctgatgcagcgacgccgc |
| | | gtgagcgaagaagtatttcggtatgtaaagctctatcagcagggaagaagaatgacggtacctgactaagaagcaccggctaa |
| | | atacgtgccagcagccgcggtaatacgtatggtgcaagcgttatccggatttactgggtgtaaagggagcgcaggcggtacg |
| | | gcaagtctgatgtgaaatcccggggctcaaccccggtactgcattggaaactgtcggactagagtgtcggaggggtaagtgg |
| | | aattcctagtgtagcggtgaaatgcgtagatattaggaggaacaccagtggcgaaggcggcttactggacgattactgacgct |
| | | gaggctcgaaagcgtggggagcaaacaggattagataccctggtagtccacgccgtaaacgatgaatactaggtgtcgggg |
| | | agcattgctcttcggtgccgcagcaaacgcaataagtattccacctggggagtacgttcgcaagaatgaaactcaaaggaattg |
| | | acggggacccgcacaagcggtggagcatgtggtttaattcgaagcaacgcgaagaaccttaccaagtcttgacatcccactga |
| | | cagagtatgtaatgtactttctcttcggagcagtggtgacaggtggtgcatggttgtcgtcagctcgtgtcgtgagatgttgggtta |
| | | agtcccgcaacgagcgcaacccctattcttagtagccagcggttcggccgggcactctagggagactgccagggataacctg |
| | | gaggaaggtggggatgacgtcaaatcatcatgccccttatgacttgggctacacacgtgctacaatggcgtaaacaaagggaa |
| | | gcaatcccgcgagggggagcaaatctcaaaaataacgtctcagttcggactgtagtctgcaactcgactacacgaagctggaa |
| | | tcgctagtaatcgcgaatcagaatgtcgcggtgaatacgttcccgggtcttgtacacaccgcccgtcacaccatgggagttggt |
| | | aatgcccgaagtcagtgacccaaccgcaaggagggagctgccgaaggcaggactgataactggggtgaagtcgtaacaag |
| | | gtagccgtatcggaaggtgcggctggatcacctccttt |
| Roseburia_ | SEQ ID | atgagagtttgatcctggctcaggatgaacgctggcggcgtgcttaacacatgcaagtcgaacgaagcactttaattgatttcttc |
| hominis | NO: 154 | ggaatgaagtttttgtgactgagtggcggacgggtgagtaacgcgtgggtaacctgcctcatacagggggataacagttggaa |
| | | acgactgctaataccgcataagcgcacaggattgcatgatccagtgtgaaaaactccggtggtatgagatggacccgcgtctg |
| | | attagccagttggcggggtaacggcccaccaaagcgacgatcagtagccgacctgagagggtgaccggccacattgggact |
| | | gagacacggcccaaactcctacgggaggcagcagtggggaatattgcacaatgggggaaaccctgatgcagcgacgccgc |
| | | gtgagcgaagaagtatttcggtatgtaaagctctatcagcagggaagaagaatgacggtacctgactaagaagcaccggctaa |
| | | atacgtgccagcagccgcggtaatacgtatggtgcaagcgttatccggatttactgggtgtaaagggagcgcaggcggtacg |
| | | gcaagtctgatgtgaaatcccggggctcaaccccggtactgcattggaaactgtcggactagagtgtcggaggggtaagtgg |
| | | aattcctagtgtagcggtgaaatgcgtagatattaggaggaacaccagtggcgaaggcggcttactggacgattactgacgct |
| | | gaggctcgaaagcgtggggagcaaacaggattagataccctggtagtccacgccgtaaacgatgaatactaggtgtcgggg |
| | | agcattgctcttcggtgccgcagcaaacgcaataagtattccacctggggagtacgttcgcaagaatgaaactcaaaggaattg |
| | | acggggacccgcacaagcggtggagcatgtggtttaattcgaagcaacgcgaagaaccttaccaagtcttgacatcccactga |
| | | cagagtatgtaatgtactttctcttcggagcagtggtgacaggtggtgcatggttgtcgtcagctcgtgtcgtgagatgttgggtta |
| | | agtcccgcaacgagcgcaacccctattcttagtagccagcggttcggccgggcactctagggagactgccagggataacctg |
| | | gaggaaggtggggatgacgtcaaatcatcatgccccttatgacttgggctacacacgtgctacaatggcgtaaacaaagggaa |
| | | gcaatcccgcgagggggagcaaatctcaaaaataacgtctcagttcggactgtagtctgcaactcgactacacgaagctggaa |
| | | tcgctagtaatcgcgaatcagaatgtcgcggtgaatacgttcccgggtcttgtacacaccgcccgtcacaccatgggagttggt |
| | | aatgcccgaagtcagtgacccaaccgcaaggagggagctgccgaaggcaggactgataactggggtgaagtcgtaacaag |
| | | gtagccgtatcggaaggtgcggctggatcacctccttt |
| Roseburia_ | SEQ ID | atgagagtttgatcctggctcaggatgaacgctggcggcgtgcttaacacatgcaagtcgaacgaagcactttaattgatttcttc |
| hominis | NO: 155 | ggaatgaagtttttgtgactgagtggcggacgggtgagtaacgcgtgggtaacctgcctcatacagggggataacagttggaa |
| | | acgactgctaataccgcataagcgcacaggattgcatgatccggtgtgaaaaactccggtggtatgagatggacccgcgtctg |
| | | attagccagttggcggggtaacggcccaccaaagcgacgatcagtagccgacctgagagggtgaccggccacattgggact |
| | | gagacacggcccaaactcctacgggaggcagcagtggggaatattgcacaatgggggaaaccctgatgcagcgacgccgc |
| | | gtgagcgaagaagtatttcggtatgtaaagctctatcagcagggaagaagaatgacggtacctgactaagaagcaccggctaa |
| | | atacgtgccagcagccgcggtaatacgtatggtgcaagcgttatccggatttactgggtgtaaagggagcgcaggcggtacg |
| | | gcaagtctgatgtgaaatcccggggctcaaccccggtactgcattggaaactgtcggactagagtgtcggaggggtaagtgg |
| | | aattcctagtgtagcggtgaaatgcgtagatattaggaggaacaccagtggcgaaggcggcttactggacgattactgacgct |
| | | gaggctcgaaagcgtggggagcaaacaggattagataccctggtagtccacgccgtaaacgatgaatactaggtgtcgggg |
| | | agcattgctcttcggtgccgcagcaaacgcaataagtattccacctggggagtacgttcgcaagaatgaaactcaaaggaattg |
| | | acggggacccgcacaagcggtggagcatgtggtttaattcgaagcaacgcgaagaaccttaccaagtcttgacatcccactga |
| | | cagagtatgtaatgtactttctcttcggagcagtggtgacaggtggtgcatggttgtcgtcagctcgtgtcgtgagatgttgggtta |
| | | agtcccgcaacgagcgcaacccctattcttagtagccagcggttcggccgggcactctagggagactgccagggataacctg |
| | | gaggaaggtggggatgacgtcaaatcatcatgccccttatgacttgggctacacacgtgctacaatggcgtaaacaaagggaa |
| | | gcaatcccgcgagggggagcaaatctcaaaaataacgtctcagttcggactgtagtctgcaactcgactacacgaagctggaa |
| | | tcgctagtaatcgcgaatcagaatgtcgcggtgaatacgttcccgggtcttgtacacaccgcccgtcacaccatgggagttggt |
| | | aatgcccgaagtcagtgacccaaccgcaaggagggagctgccgaaggcaggactgataactggggtgaagtcgtaacaag |
| | | gtagccgtatcggaaggtgcggctggatcacctccttt |
| Erysipelotrichaceae_ | SEQ ID | atggagagtttgatcctggctcaggatgaacgctggcggcatgcctaatacatgcaagtcgaacgaagcttcaggaagcttgct |
| bacterium_2_2_ | NO: 156 | tccaaagagacttagtggcgaacgggtgagtaacacgtaggtaacctgcccatgtgtccgggataactgctggaaacggtag |
| 44A | | ctaaaaccggataggtatacagagcgcatgctcagtatattaaagcgcccatcaaggcgtgaacatggatggacctgcggcg |
| | | cattagctagttggtgaggtaacggcccaccaaggcgatgatgcgtagccggcctgagagggtaaacggccacattgggact |
| | | gagacacggcccaaactcctacgggaggcagcagtagggaattttcgtcaatgggggaaaccctgaacgagcaatgccgc |
| | | gtgagtgaagaaggtcttcggatcgtaaagctctgttgtaagtgaagaacggctcatagaggaaatgctatgggagtgacggt |
| | | agcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggaaacaaataccctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccctttatggcctgggctacacacgtactacaatggcga |
| | | ccacaaagagcagcgacttggtgacaagaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Clostridium_ | SEQ ID | tttgagagtttgatcctggctcaggatgaacgctggcggcgtgcctaacacatgcaagtcgaacgaagcaatcagaatgaaatt |
| citroniae | NO: 157 | ttcggatggatttctggttgactgagtggcggacgggtgagtaacgcgtggataacctgcctcacactgggggataacagttag |
| | | aaatggctgctaataccgcataagcgcacagtaccgcatggtacggtgtgaaaaactccggtggtgtgagatggatccgcgtc |
| | | tgattagttagaggcggggtaacggcccaccaagacagcgatcagtagccgacctgagagggtgaccggccacattggga |
| | | ctgagacacggcccaaactcctacgggaggcagcagtggggaatattgcacaatgggcgaaagcctgatgcagcgacgcc |
| | | gcgtgagtgaagaagtatttcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgtagacggcg |
| | | aagcaagtctgaagtgaaaacccagggctcaaccctgggactgctttggaaactgttttgctagagtgtcggagaggtaagtg |
| | | gaattcctagtgtagcggtgaaatgcgtagatattaggaggaacaccagtggcgaaggcggcttactggacgataactgacgt |
| | | tgaggctcgaaagcgtggggagcaaacaggattagataccctggtagtccacgccgtaaacgatgaatgctaggtgttgggg |
| | | ggcaaagcccttcggtgccgtcgcaaacgcaataagcattccacctggggagtacgttcgcaagaatgaaactcaaaggaatt |
| | | gacggggacccgcacaagcggtggagcatgtggtttaattcgaagcaacgcgaagaaccttaccaagtcttgacatccccct |
| | | gaccggtcagtaaagtgacctttccttcgggacaggggagacaggtggtgcatggttgtcgtcagctcgtgtcgtgagatgttg |
| | | ggttaagtcccgcaacgagcgcaacccttatccttagtagccagcaggtaaagctgggcactctagggagactgccagggat |
| | | aacctggaggaaggtggggatgacgtcaaatcatcatgccccttatgatttgggctacacacgtgctacaatggcgtaaacaaa |
| | | gggaagcgaccctgcgaaggcaagcaaatcccaaaaataacgtcccagttcggactgtagtctgcaacccgactacacgaa |
| | | gctggaatcgctagtaatcgcgaatcagaatgtcgcggtgaatacgttcccgggtcttgtacacaccgcccgtcacaccatgg |
| | | gagtcagcaacgcccgaannnnn |
| Flavonifractor_ | SEQ ID | tattgagagtttgatcctggctcaggatgaacgctggcggcgtgcttaacacatgcaagtcgaacggggtgctcatgacggag |
| plautii | NO: 158 | gattcgtccaatggattgagttacctagtggcggacgggtgagtaacgcgtgaggaacctgccttggagaggggaataacact |
| | | ccgaaaggagtgctaataccgcatgaagcagttgggtcgcatggctctgactgccaaagatttatcgctctgagatggcctcgc |
| | | gtctgattagctagtaggcggggtaacggcccacctaggcgacgatcagtagccggactgagaggttgaccggccacattgg |
| | | gactgagacacggcccagactcctacgggaggcagcagtggggaatattgggcaatgggcgcaagcctgacccagcaac |
| | | gccgcgtgaaggaagaaggctttcgggttgtaaacttcttttgtcggggacgaaacaaatgacggtacccgacgaataagcca |
| | | cggctaactacgtgccagcagccgcggtaatacgtaggtggcaagcgttatccggatttactgggtgtaaagggcgtgtaggc |
| | | gggattgcaagtcagatgtgaaaactgggggctcaacctccagcctgcatttgaaactgtagttcttgagtgctggagaggcaa |
| | | tcggaattccgtgtgtagcggtgaaatgcgtagatatacggaggaacaccagtggcgaaggcggattgctggacagtaactg |
| | | acgctgaggcgcgaaagcgtggggagcaaacaggattagataccctggtagtccacgccgtaaacgatggatactaggtgt |
| | | ggggggtctgaccccctccgtgccgcagttaacacaataagtatcccacctggggagtacgatcgcaaggttgaaactcaaa |
| | | ggaattgacgggggcccgcacaagcggtggagtatgtggtttaattcgaagcaacgcgaagaaccttaccagggcttgacat |
| | | cccactaacgaggcagagatgcgttaggtgcccttcggggaaagtggagacaggtggtgcatggttgtcgtcagctcgtgtc |
| | | gtgagatgttgggttaagtcccgcaacgagcgcaacccttattgttagttgctacgcaagagcactctagcgagactgccgttg |
| | | acaaaacggaggaaggtggggacgacgtcaaatcatcatgccccttatgtcctgggccacacacgtactacaatggtggttaa |
| | | cagagggaggcaataccgcgaggtggagcaaatccctaaaagccatcccagttcggattgcaggctgaaacccgcctgtatg |
| | | aagttggaatcgctagtaatcgcggatcagcatgccgcggtgaatacgttcccgggccttgtacacaccgcccgtcacaccat |
| | | gagagtcgggaacacccgaagtccgtagcctaaccgcaaggagggcgcggccgaaggtgggttcgataattggggtgaag |
| | | tcgtaacaaggtagccgtatcggaaggtgcggctggatcacctccttt |
| Lachnospiraceae_ | SEQ ID | tattgagagtttgatcctggctcaggatgaacgctggcggcgtgcttaacacatgcaagtcgaacggggtgctcatgacggag |
| bacterium_ | NO: 159 | gattcgtccaacggattgagttacctagtggcggacgggtgagtaacgcgtgaggaacctgccttggagaggggaataacac |
| 7_1_58 | | tccgaaaggagtgctaataccgcatgatgcagttgggtcgcatggctctgactgccaaagatttatcgctctgagatggcctcg |
| FAA | | cgtctgattagctagtaggcggggtaacggcccacctaggcgacgatcagtagccggactgagaggttgaccggccacattg |
| | | ggactgagacacggcccagactcctacgggaggcagcagtggggaatattgggcaatgggcgcaagcctgacccagcaa |
| | | cgccgcgtgaaggaagaaggctttcgggttgtaaacttcttttgtcggggacgaaacaaatgacggtacccgacgaataagcc |
| | | acggctaactacgtgccagcagccgcggtaatacgtaggtggcaagcgttatccggatttactgggtgtaaagggcgtgtagg |
| | | cgggattgcaagtcagatgtgaaaactgggggctcaacctccagcctgcatttgaaactgtagttcttgagtgctggagaggca |
| | | atcggaattccgtgtgtagcggtgaaatgcgtagatatacggaggaacaccagtggcgaaggcggattgctggacagtaact |
| | | gacgctgaggcgcgaaagcgtggggagcaaacaggattagataccctggtagtccacgccgtaaacgatggatactaggtg |
| | | tggggggtctgaccccctccgtgccgcagttaacacaataagtatcccacctggggagtacgatcgcaaggttgaaactcaaa |
| | | ggaattgacgggggcccgcacaagcggtggagtatgtggtttaattcgaagcaacgcgaagaaccttaccagggcttgacat |
| | | cccactaacgaagcagagatgcattaggtgcccttcggggaaagtggagacaggtggtgcatggttgtcgtcagctcgtgtcg |
| | | tgagatgttgggttaagtcccgcaacgagcgcaacccttattgttagttgctacgcaagagcactctagcgagactgccgttgac |
| | | aaaacggaggaaggtggggacgacgtcaaatcatcatgccccttatgtcctgggccacacacgtactacaatggtggttaaca |
| | | gagggaggcaataccgcgaggtggagcaaatccctaaaagccatcccagttcggattgcaggctgaaacccgcctgtatgaa |
| | | gttggaatcgctagtaatcgcggatcagcatgccgcggtgaatacgttcccgggccttgtacacaccgcccgtcacaccatga |
| | | gagtcgggaacacccgaagtccgtagcctaaccgcaaggagggcgcggccgaaggtgggttcgataattggggtgaagtc |
| | | gtaacaaggtagccgtatcggaaggtgcggctggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 160 | cttccaaagagacttagtggcgaacgggtgagtaacacgtaggtaacctgcccatgtgtccgggataactgctggaaacggta |
| 6_1_45 | | gctaaaaccggataggtatacagagcgcatgctcagtatattaaagcgnnnnnnnnnnnnnnnnnnnnnnnnnnnnnnn |
| | | nnnnnnnnnnnnnnnnnnnnnnnnnnnnnnnnnnnnnnnnnnnnnnnnnnnnnnnnnnnnnnnnnnnnnng |
| | | aggtaacggctcaccaaggcgatgatgcgtagccggcctgagagggtaaacggccacattgggactgagacacggcccaa |
| | | actcctacgggaggcagcagtagggaattttcgtcaatgggggaaaccctgaacgagcaatgccgcgtgagtgaagaaggt |
| | | cttcggatcgtaaagctctgttgtaagtgaagaacggatcatagaggaaatgctatgggagtgacggtagcttaccagaaagcc |
| | | acggctaactacgtgccagcagccgcggtaatacgtaggtggcaagcgttatccggaatcattgggcgtaaagggtgcgtag |
| | | gtggcgtactaagtctgtagtaaaaggcaatggctcaaccattgtaagctatggaaactggtatgctggagtgcagaagaggg |
| | | cgatggaattccatgtgtagcggtaaaatgcgtagatatatggaggaacaccagtggcgaaggcggtcgcctggtctgtaact |
| | | gacactgaggcacgaaagcgtggggagcaaataggattagataccctagtagtccacgccgtaaacgatgagaactaagtgt |
| | | tggaggaattcagtgctgcagttaacgcaataagttctccgcctggggagtatgcacgcaagtgtgaaactcaaaggaattgac |
| | | gggggcccgcacaagcggtggagtatgtggtttaattcgaagcaacgcgaagaaccttaccaggccttgacatggaaacaaa |
| | | taccctagagatagggggataattatggatcacacaggtggtgcatggttgtcgtcagctcgtgtcgtgagatgttgggttaagt |
| | | cccgcaacgagcgcaacccttgtcgcatgttaccagcatcaagttggggactcatgcgagactgccggtgacaaaccggagg |
| | | aaggtggggatgacgtcaaatcatcatgccccttatggcctgggctacacacgtactacaatggcggccacaaagagcagcg |
| | | acacagtgatgtgaagcgaatctcataaaggtcgtctcagttcggattgaagtctgcaactcgacttcatgaagtcggaatcgct |
| | | agtaatcgcagatcagcatgctgcggtgaatacgttctcgggccttgtacacaccgcccgtcaaaccatgggagtcagtaatac |
| | | ccgaagccggtggcataaccgtaaggagtgagccgtcgaaggtaggaccgatgactggggttaagtcgtaacaaggtatcc |
| | | ctacgggaacgtggggatggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 161 | cttccaaagagacttagtggcgaacgggtgagtaacacgtaggtaacctgcccatgtgtccgggataactgctggaaacggta |
| 6_1_45 | | gctaaaaccggataggtatacagagcgcatgctcagtatattaaagcgtccatcaaggcgtgaacatggatggacctgcggcg |
| | | cattagctagttggtgaggtaacggctcaccaaggcgatgatgcgtagccggcctgagagggtaaacggccacattgggact |
| | | gagacacggcccaaactcctacgggaggcagcagtagggaattttcgtcaatgggggaaaccctgaacgagcaatgccgc |
| | | gtgagtgaagaaggtcttcggatcgtaaagctctgttgtaagtgaagaacggctcatagaggaaatgctatgggagtgacggt |
| | | agcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcttggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggaaacaaataccctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcgg |
| | | ccacaaagagcagcgacacagtgatgtgaagcgaatctcataaaggtcgtctcagttcggattgaagtctgcaactcgacttca |
| | | tgaagtcggaatcgctagtaatcgcagatcagcatgctgcggtgaatacgactcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 162 | cttccaaagagacttagtggcgaacgggtgagtaacacgtaggtaacctgcccatgtgtccgggataactgctggaaacggta |
| 6_1_45 | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggatcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggaaacaaataccctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcgg |
| | | ccacaaagagcagcgacacagtgatgtgaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 163 | cttccaaagagacttagtggcgaacgggtgagtaacacgtaggtaacctgcccatgtgtccgggataactgctggaaacggta |
| 6_1_45 | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggatcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggaaacaaataccctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcgg |
| | | ccacaaagagcagcgacacagtgatgtgaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 164 | cttccaaagagacttagtggcgaacgggtgagtaacacgtaggtaacctgcccatgtgtccgggataactgctggaaacggta |
| 21_3 | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggctcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggatgcaaatgttctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcgg |
| | | ccacaaagagcagcgacacagtgatgtgaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 165 | cttccaaagagacttagtggcgaacgggtgagtaacacgtaggtaacctgcccatgtgtccgggataactgctggaaacggta |
| 21_3 | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggctcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacat |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 166 | cttccaaagagacttagtggcgaacgggtgagtaacacgtaggtaacctgcccatgtgtccgggataactgctggaaacggta |
| 21_3 | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggctcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggatgcaaatgttctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcgg |
| | | ccacaaagagcagcgacacagtgatgtgaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 167 | cttccaaagagacttagtggcgaacgggtgagtaacacgtaggtaacctgcccatgtgtccgggataactgctggaaacggta |
| 21_3 | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggctcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggatgcaaatgttctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcgg |
| | | ccacaaagagcagcgacacagtgatgtgaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Erysipelotrichaceae_ | SEQ ID | atggagagtttgatcctggctcaggatgaacgctggcggcatgcctaatacatgcaagtcgaacgaagtttcgaggaagcttg |
| bacterium_ | NO: 168 | cttccaaagagacttagtggcgaacgggtgagtaacacgtaggtaacctgcccatgtgtccgggataactgctggaaacggta |
| 21_3 | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggctcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggatgcaaatgttctagagatagggggataattatggatcacacaggtggtgcatggttgtcgtcagacgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcgg |
| | | ccacaaagagcagcgacacagtgatgtgaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Haemophilus_ | SEQ ID | ttgaagagtttgatcatggctcagattgaacgctggcggcaggcttaacacatgcaagtcgaacggtaacataaagaagcttgc |
| parainfluenzae | NO: 169 | ttctttgatgacgagtguggacgggtgagtaatgcttgggaatctagcttatggagggggataactacgggaaactgtagcta |
| | | ataccgcgtagaatcgggagatgaaagtgtgggaccttcgggccacatgccataggatgagcccaagtgggattaggtagtt |
| | | ggtgaggtaaaggctcaccaagccgacgatctctagctggtctgagaggatgaccagccacactgggactgagacacggcc |
| | | cagactcctacgggaggcagcagtggggaatattgcgcaatgggggcaaccctgacgcagccatgccgcgtgaatgaaga |
| | | aggccttcgggttgtaaagttctttcggtagcgaggaaggcatttagtttaataaactggatgattgacgttaactacagaagaag |
| | | caccggctaactccgtgccagcagccgcggtaatacggagggtgcgagcgttaatcggaataactgggcgtaaagggcacg |
| | | caggcggtgacttaagtgaggtgtgaaagccccgggcttaacctgggaattgcatttcatactgggtcgctagagtactttagg |
| | | gaggggtagaattccacgtgtagcggtgaaatgcgtagagatgtggaggaataccgaaggcgaaggcagccccttgggaat |
| | | gtactgacgctcatgtgcgaaagcgtggggagcaaacaggattagataccctggtagtccacgctgtaaacgatgtcgatttg |
| | | ggggttgagctttaagtttggcgcccgtagctaacgtgataaatcgaccgcctggggagtacggccgcaaggttaaaactcaa |
| | | atgaattgacgggggcccgcacaagcggtggagcatgtggtttaattcgatgcaacgcgaagaaccttacctactcttgacatc |
| | | cagagaacattccagagatggattggtgccttcgggaactctgagacaggtgctgcatggctgtcgtcagctcgtgttgtgaaa |
| | | tgttgggttaagtcccgcaacgagcgcaacccttatcctttgagccagcgatttggtcgggaactcaaaggagactgccggtg |
| | | ataaaccggaggaaggtggggatgacgtcaagtcatcatggcccttacgagtagggctacacacgtgctacaatggcgtata |
| | | cagagggaagcgagagtgcgagctggagcgaatctcacaaagtacgtctaagtccggattggagtctgcaactcgactccat |
| | | gaagtcggaatcgctagtaatcgcaaatcagaatgttgcggtgaatacgttcccgggccttgtacacaccgcccgtcacaccat |
| | | gggagtgggttgtaccagaagtagatagcttaaccacgggggggcgtttaccacggtatgattcatgactggggtgaagtcgt |
| | | aacaaggtaaccgtaggggaacctgcggttggatcacctcctt |
| Streptococcus_ | SEQ ID | taatgagagtttgatcctggctcaggatgaacgctggcggcgtgcctaatacatgcaagtagaacgctgaagcttggtgcttgc |
| parasanguinis | NO: 170 | accgagcggaggagttgcgaacgggtgagtaacgcgtaggtaacctgcctcttagcgggggataactattggaaacgatagc |
| | | taataccgcataaaagtcgacattgcatgatgttgacttgaaaggtgcaactgcatcactaagagatggacctgcgttgtattagc |
| | | tagttggtgaggtaacggctcaccaaggcgacgatacatagccgacctgagagggtgatcggccacactgggactgagaca |
| | | cggcccagactcctacgggaggcagcagtagggaatcttcggcaatggacggaagtctgaccgagcaacgccgcgtgagt |
| | | gaagaaggttttcggatcgtaaagctctgagtaagagaagaacgagtgtgagagtggaaagttcacactgtgacggtaactta |
| | | ccagaaagggacggctaactacgtgccagcagccgcggtaatacgtaggtcccgagcgttatccggatttattgggcgtaaa |
| | | gcgagcgcaggcggttagataagtctgaagttaaaggctgtggcttaaccatagtacgctttggaaactgtttaacttgagtgca |
| | | gaaggggagagtggaattccatgtgtagcggtgaaatgcgtagatatatggaggaacaccggtggcgaaagcggctctctgg |
| | | tctgtaactgacgctgaggctcgaaagcgtggggagcaaacaggattagataccctggtagtccacgccgtaaacgatgagt |
| | | gctaggtgttgggtcctttccgggactcagtgccgcagctaacgcattaagcactccgcctggggagtacgaccgcaaggttg |
| | | aaactcaaaggaattgacgggggcccgcacaagcggtggagcatgtggtttaattcgaagcaacgcgaagaaccttaccag |
| | | gtcttgacatccctctgaccgctctagagatagagctttccttcgggacagaggtgacaggtggtgcatggttgtcgtcagctcg |
| | | tgtcgtgagatgttgggttaagtcccgcaacgagcgcaacccctattgttagttgccatcatttagagggcactctagcgagact |
| | | gccggtaataaaccggaggaaggtggggatgacgtcaaatcatcatgccccttatgacctgggctacacacgtgctacaatgg |
| | | ctggtacaacgagtcgcgagtcggtgacggcaagctaatctcttaaagccagtctcagttcggattgtaggctgcaactcgcct |
| | | acatgaagtcggaatcgctagtaatcgcggatcagcacgccgcggtgaatacgttcccgggccttgtacacaccgcccgtcac |
| | | accacgagagtttgtaacacccgaagtcggtgaggtaaccttttaggagccagccgcctaaggtgggatagatgattggggtg |
| | | aagtcgtaacaaggtagccgtatcggaaggtgcggctggatcacctccttt |
| Streptococcus_ | SEQ ID | taatgagagtttgatcctggctcaggatgaacgctggcggcgtgcctaatacatgcaagtagaacgctgaagcttggtgcttgc |
| parasanguinis | NO: 171 | accgagcggaggagttgcgaacgggtgagtaacgcgtaggtaacctgcctcttagcgggggataactattggaaacgatagc |
| | | taataccgcataaaagtcgacattgcatgatgttgacttgaaaggtgcaaatgcatcactgagagatggacctgcgttgtattagc |
| | | tagttggtgaggtaacggctcaccaaggcgacgatacatagccgacctgagagggtgatcggccacactgggactgagaca |
| | | cggcccagactcctacgggaggcagcagtagggaatcttcggcaatggacggaagtctgaccgagcaacgccgcgtgagt |
| | | gaagaaggttttcggatcgtaaagctctgttgtaagagaagaacgagtgtgagagtggaaagttcacactgtgacggtaactta |
| | | ccagaaagggacggctaactacgtgccagcagccgcggtaatacgtaggtcccgagcgttatccggatttattgggcgtaaa |
| | | gcgagcgcaggcggttagataagtctgaagttaaaggctgtggcttaaccatagtacgctttggaaactgtttaacttgagtgca |
| | | gaaggggagagtggaattccatgtgtagcggtgaaatgcgtagatatatggaggaacaccggtggcgaaagcggctctctgg |
| | | tctgtaactgacgctgaggctcgaaagcgtggggagcaaacaggattagataccctggtagtccacgccgtaaacgatgagt |
| | | gctaggtgttggtcctttccgggactcagtgccgcagctaacgcattaagcactccgcctggggagtacgaccgcaaggttga |
| | | aactcaaaggaattgacgggggcccgcacaagcggtggagcatgtggtttaattcgaagcaacgcgaagaaccttaccaggt |
| | | cttgacatccctctgaccgctctagagatagagctttccttcgggacagaggtgacaggtggtgcatggttgtcgtcagctcgtg |
| | | tcgtgagatgttgggttaagtcccgcaacgagcgcaacccctattgttagttgccatcatttagttgggcactctagcgagactgc |
| | | cggtaataaaccggaggaaggtggggatgacgtcaaatcatcatgccccttatgacctgggctacacacgtgctacaatggct |
| | | ggtacaacgagtcgcgagtcggtgacggcaagctaatctcttaaagccagtctcagttcggattgtaggctgcaactcgcctac |
| | | atgaagtcggaatcgctagtaatcgcggatcagcacgccgcggtgaatacgttcccgggccttgtacacaccgcccgtcacac |
| | | cacgagagtttgtaacacccgaagtcggtgaggtaaccttttaggagccagccgcctaaggtgggatagatgattggggtgaa |
| | | gtcgtaacaaggtagccgtatcggaaggtgcggctggatcacctccttt |
| Streptococcus_ | SEQ ID | taatgagagtttgatcctggctcaggatgaacgctggcggcgtgcctaatacatgcaagtagaacgctgaagcttggtgcttgc |
| parasanguinis | NO: 172 | accgagcggaggagttgcgaacgggtgagtaacgcgtaggtaacctgcctcttagcgggggataactattggaaacgatagc |
| | | taataccgcataaaagtcgacattgcatgatgttgacttgaaaggtgcaactgcatcactaagagatggacctgcgttgtattagc |
| | | tagttggtgaggtaacggctcaccaaggcgacgatacatagccgacctgagagggtgatcggccacactgggactgagaca |
| | | cggcccagactcctacgggaggcagcagtagggaatcttcggcaatggacggaagtctgaccgagcaacgccgcgtgagt |
| | | gaagaaggttttcggatcgtaaagctctgttgtaagagaagaacgagtgtgagagtggaaagttcacactgtgacggtaactta |
| | | ccagaaagggacggctaactacgtgccagcagccgcggtaatacgtaggtcccgagcgttatccggatttattgggcgtaaa |
| | | gcgagcgcaggcggttagataagtctgaagttaaaggctgtggcttaaccatagtacgctttggaaactgtttaacttgagtgca |
| | | gaaggggagagtggaattccatgtgtagcggtgaaatgcgtagatatatggaggaacaccggtggcgaaagcggctctctgg |
| | | tctgtaactgacgctgaggctcgaaagcgtggggagcaaacaggattagataccctggtagtccacgccgtaaacgatgagt |
| | | gctaggtgttgggtcctttccgggactcagtgccgcagctaacgcattaagcactccgcctggggagtacgaccgcaaggttg |
| | | aaactcaaaggaattgacgggggcccgcacaagcggtggagcatgtggtttaattcgaagcaacgcgaagaaccttaccag |
| | | gtcttgacatccctctgaccgctctagagatagagctttccttcgggacagaggtgacaggtggtgcatggttgtcgtcagctcg |
| | | tgtcgtgagatgttgggttaagtcccgcaacgagcgcaacccctattgttagttgccatcatttagttgggcactctagcgagact |
| | | gccggtaataaaccggaggaaggtggggatgacgtcaaatcatcatgccccttatgacctgggctacacacgtgctacaatgg |
| | | ctggtacaacgagtcgcgagtcggtgacggcaagctaatctcttaaagccagtctcagttcggattgtaggctgcaactcgcct |
| | | acatgaagtcggaatcgctagtaatcgcggatcagcacgccgcggtgaatacgttcccgggccttgtacacaccgcccgtcac |
| | | accacgagagtttgtaacacccgaagtcggtgaggtaaccttttaggagccagccgcctaaggtgggatagatgattggggtg |
| | | aagtcgtaacaaggtagccgtatcggaaggtgcggctggatcacctccttt |
| Streptococcus_ | SEQ ID | aatgagagtttgatcctggctcaggatgaacgctggcggcgtgcctaatacatgcaagtagaacgctgaagcttggtgcttgca |
| parasanguinis | NO: 173 | ccgagcggatgagttgcgaacgggtgagtaacgcgtaggtaacctacctcttagcgggggataactattggaaacgatagcta |
| | | ataccgcataaaagtcggcattgcatgatgagacttgaaaggtgcaaatgcatcactgagagatggacctgcgttgtattagct |
| | | agttggtgaggtaacggctcaccaaggcgacgatacatagccgacctgagagggtgatcggccacactgggactgagacac |
| | | ggcccagactcctacgggaggcagcagtagggaatcttcggcaatggacggaagtctgaccgagcaacgccgcgtgagtg |
| | | aagaaggttttcggatcgtaaagctctgttgtaagagaagaacgagtgtgagagtggaaagttcacactgtgacggtaacttac |
| | | cagaaagggacggctaactacgtgccagcagccgcggtaatacgtaggtcccgagcgttatccggatttattgggcgtaaag |
| | | cgagcgcaggcggttagataagtctgaagttaaaggctgtggcttaaccatagtacgctttggaaactgtttaacttgagtgcag |
| | | aaggggagagtggaattccatgtgtagcggtgaaatgcgtagatatatggaggaacaccggtggcgaaagcggctctctggt |
| | | ctgtaactgacgctgaggctcgaaagcgtggggagcaaacaggattagataccctggtagtccacgccgtaaacgatgagtg |
| | | ctaggtgttgggtcctttccgggactcagtgccgcagctaacgcattaagcactccgcctggggagtacgaccgcaaggttga |
| | | aactcaaaggaattgacgggggcccgcacaagcggtggagcatgtggtttaattcgaagcaacgcgaagaaccttaccaggt |
| | | cttgacatccctctgaccgctctagagatagagctttccttcgggacagaggtgacaggtggtgcatggttgtcgtcagctcgtg |
| | | tcgtgagatgttgggttaagtcccgcaacgagcgcaacccctattgttagttgccatcatttagttgggcactctagcgagactgc |
| | | cggtaataaaccggaggaaggtggggatgacgtcaaatcatcatgccccttatgacctgggctacacacgtgctacaatggct |
| | | ggtacaacgagtcgcgagtcggtgacggcaagctaatctcttaaagccagtctcagttcggattgtaggctgcaactcgcctac |
| | | atgaagtcggaatcgctagtaatcgcggatcagcacgccgcggtgaatacgttcccgggccttgtacacaccgcccgtcacac |
| | | cacgagagtttgtaacacccgaagtcggtgaggtaaccttttaggagccagccgcctaaggtgggatagatgattggggtgaa |
| | | gtcgtaacaaggtagccgtatcggaaggtgcggctggatcacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 174 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 175 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 176 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggagtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 177 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 178 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattaccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 179 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 180 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattaccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgctttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 181 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 182 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| dorei | NO: 183 | ctaaggctgatggcgaccggcgcacgggtgagtaacacgtatccaacctgccgtctactcttggccagccttctgaaaggaag |
| | | attaatccaggatgggatcatgagttcacatgtccgcatgattaaaggtattttccggtagacgatggggatgcgttccattagat |
| | | agtaggcggggtaacggcccacctagtcaacgatggataggggttctgagaggaaggtcccccacattggaactgagacac |
| | | ggtccaaactcctacgggaggcagcagtgaggaatattggtcaatgggcgatggcctgaaccagccaagtagcgtgaagga |
| | | tgactgccctatgggttgtaaacttcttttataaaggaataaagtcgggtatgcatacccgtttgcatgtactttatgaataaggatc |
| | | ggctaactccgtgccagcagccgcggtaatacggaggatccgagcgttatccggatttattgggtttaaagggagcgtagatg |
| | | gatgtttaagtcagttgtgaaagtttgcggctcaaccgtaaaattgcagttgatactggatgtcttgagtgcagttgaggcaggcg |
| | | gaattcgtggtgtagcggtgaaatgcttagatatcacgaagaactccgattgcgaaggcagcctgctaagctgcaactgacatt |
| | | gaggctcgaaagtgtgggtatcaaacaggattagataccctggtagtccacacggtaaacgatgaatactcgctgtttgcgatat |
| | | acggcaagcggccaagcgaaagcgttaagtattccacctggggagtacgccggcaacggtgaaactcaaaggaattgacg |
| | | ggggcccgcacaagcggaggaacatgtggtttaattcgatgatacgcgaggaaccttacccgggcttaaattgcactcgaatg |
| | | atccggaaacggttcagctagcaatagcgagtgtgaaggtgctgcatggttgtcgtcagctcgtgccgtgaggtgtcggcttaa |
| | | gtgccataacgagcgcaacccttgttgtcagttactaacaggtgatgctgaggactctgacaagactgccatcgtaagatgtga |
| | | ggaaggtggggatgacgtcaaatcagcacggcccttacgtccggggctacacacgtgttacaatggggggtacagagggcc |
| | | gctaccacgcgagtggatgccaatccctaaaacccctctcagttcggactggagtctgcaacccgactccacgaagctggatt |
| | | cgctagtaatcgcgcatcagccacggcgcggtgaatacgttcccgggccttgtacacaccgcccgtcaagccatgggagccg |
| | | ggggtacctgaagtgcgtaaccgcgaggatcgccctagggtaaaactggtgactggggctaagtcgtaacaaggtagccgta |
| | | ccggaaggtgcggctggaacacctcctt |
| Bacteroides_ | SEQ ID | atgaagagtttgatcctggctcaggatgaacgctagctacaggcttaacacatgcaagtcgaggggcatcaggaagaaagctt |
| fragilis | NO: 184 | gctttctttgctggcgaccggcgcacgggtgagtaacacgtatccaacctgccctttactcggggatagcctttcgaaagaaag |
| | | attaatacccgatagcataatgattccgcatggtttcattattaaaggattccggtaaaggatggggatgcgttccattaggttgttg |
| | | gtgaggtaacggctcaccaagccttcgatggataggggttctgagaggaaggtcccccacattggaactgagacacggtcca |
| | | aactcctacgggaggcagcagtgaggaatattggtcaatgggcgttagcctgaaccagccaagtagcgtgaaggatgaagg |
| | | ctctatgggtcgtaaacttcttttatataagaataaagtgcagtatgtatactgttttgtatgtattatatgaataaggatcggctaactc |
| | | cgtgccagcagccgcggtaatacggaggatccgagcgttatccggatttattgggtttaaagggagcgtaggtggactggtaa |
| | | gtcagttgtgaaagtttgcggctcaaccgtaaaattgcagttgatactgtcagtcttgagtacagtagaggtgggcggaattcgtg |
| | | gtgtagcggtgaaatgcttagatatcacgaagaactccgattgcgaaggcagctcactggactgcaactgacactgatgctcg |
| | | aaagtgtgggtatcaaacaggattagataccctggtagtccacacagtaaacgatgaatactcgctgtttgcgatatacagtaag |
| | | cggccaagcgaaagcattaagtattccacctggggagtacgccggcaacggtgaaactcaaaggaattgacgggggcccg |
| | | cacaagcggaggaacatgtggtttaattcgatgatacgcgaggaaccttacccgggcttaaattgcagtggaatgatgtggaaa |
| | | catgtcagtgagcaatcaccgctgtgaaggtgctgcatggttgtcgtcagctcgtgccgtgaggtgtcggcttaagtgccataa |
| | | cgagcgcaacccttatcttcagttactaacaggtcatgctgaggactctggagagactgccgtcgtaagatgtgaggaaggtg |
| | | gggatgacgtcaaatcagcacggcccttacgtccggggctacacacgtgttacaatggggggtacagaaggcagctagcgg |
| | | gtgaccgtatgctaatcccaaaagcctctctcagttcggatcgaagtctgcaacccgacttcgtgaagctggattcgctagtaat |
| | | cgcgcatcagccacggcgcggtgaatacgttcccgggccttgtacacaccgcccgtcaagccatgggagccgggggtacct |
| | | gaagtacgtaaccgcaaggatcgtcctagggtaaaactggtgactggggctaagtcgtaacaaggtagccgtaccggaaggt |
| | | gcggctggaacacctcctt |
| Bacteroides_ | SEQ ID | aaaggatggggatgcgttccattaggttgttggtgaggtaacggctcaccaagccttcgatggataggggttctgagaggaag |
| fragilis | NO: 185 | gtcccccacattggaactgagacacggtccaaactcctacgggaggcagcagtgaggaatattggtcaatgggcgctagcct |
| | | gaaccagccaagtagcgtgaaggatgaaggctctatgggtcgtaaacttcttttatataagaataaagtgcagtatgtatactgttt |
| | | tgtatgtattatatgaataaggatcggctaactccgtgccagcagccgcggtaatacggaggatccgagcgttatccggatttatt |
| | | gggtttaaagggagcgtaggtggactggtaagtcagttgtgaaagtttgcggctcaaccgtaaaattgcagttgatactgtcagt |
| | | cttgagtacagtagaggtgggcggaattcgtggtgtagcggtgaaatgcttagatatcacgaagaactccgattgcgaaggca |
| | | gctcactggactgcaactgacactgatgctcgaaagtgtgggtatcaaacaggattagataccctggtagtccacacagtaaac |
| | | gatgaatactcgctgtttgcgatatacagtaagcggccaagcgaaagcattaagtattccacctggggagtacgccggcaacg |
| | | gtgaaactcaaaggaattgacgggggcccgcacaagcggaggaacatgtggtttaattcgatgatacgcgaggaaccttacc |
| | | cgggcttaaattgcagtggaatgatgtggaaacatgtcagtgagcaatcaccgctgtgaaggtgctgcatggttgtcgtcagct |
| | | cgtgccgtgaggtgtcggcttaagtgccataacgagcgcaacccttatcttcagttactaacaggttatgctgaggactctggag |
| | | agactgccgtcgtaagatgtgaggaaggtggggatgacgtcaaatcagcacggcccttacgtccggggctacacacgtgtta |
| | | caatggggggtacagaaggcagctagcgggtgaccgtatgctaatcccaaaagcctctctcagttcggatcgaagtctgcaac |
| | | ccgacttcgtgaagctggattcgctagtaatcgcgcatcagccacggcgcggtgaatacgttcccgggccttgtacacaccgc |
| | | ccgtcaagccatgggagccgggggtacctgaagtacgtaaccgcaaggatcgtcctagggtaaaactggtgactggggcta |
| | | agtcgtaacaaggtagccgtaccggaaggtgcggctggaacacctcctt |
| Bacteroides_ | SEQ ID | atgaagagtttgatcctggctcaggatgaacgctagctacaggcttaacacatgcaagtcgaggggcatcaggaagaaagctt |
| fragilis | NO: 186 | gctttctttgctggcgaccggcgcacgggtgagtaacacgtatccaacctgccctttactcggggatagcctttcgaaagaaag |
| | | attaatacccgatagtataatgattccgcatggtttcattattaaaggattccggtaaaggatggggatgcgttccattaggttgttg |
| | | gtgaggtaacggcccaccaagccttcgatggataggggttctgagaggaaggtcccccacattggaactgagacacggtcca |
| | | aactcctacgggaggcagcagtgaggaatattggtcaatgggcgttagcctgaaccagccaagtagcgtgaaggatgaagg |
| | | ctctatgggtcgtaaacttcttttatataagaataaagtgcagtatgttttactgttttgtatgtattatatgaataaggatcggctaactc |
| | | cgtgccagcagccgcggtaatacggaggatccgagcgttatccggatttattgggtttaaagggagcgtaggtggactggtaa |
| | | gtcagttgtgaaagtttgcggctcaaccgtaaaattgcagttgatactgtcagtcttgagtacagtagaggtgggcggaattcgtg |
| | | gtgtagcggtgaaatgcttagatatcacgaagaactccgattgcgaaggcagctcactggactgcaactgacactgatgctcg |
| | | aaagtgtgggtatcaaacaggattagataccctggtagtccacacagtaaacgatgaatactcgctgtttgcgatatacagtaag |
| | | cggccaagcgaaagcattaagtattccacctggggagtacgccggcaacggtgaaactcaaaggaattgacgggggcccg |
| | | cacaagcggaggaacatgtggtttaattcgatgatacgcgaggaaccttacccgggcttaaattgcagtggaatgatgtggaaa |
| | | catgtcagtgagcaatcaccgctgtgaaggtgctgcatggttgtcgtcagctcgtgccgtgaggtgtcggcttaagtgccataa |
| | | cgagcgcaacccttatcttcagttactaacaggtcatgctgaggactctggagagactgccgtcgtaagatgtgaggaaggtg |
| | | gggatgacgtcaaatcagcacggcccttacgtccggggctacacacgtgttacaatggggggtacagaaggcagctagcgg |
| | | gtgaccgtatgctaatcccaaaagcctctctcagttcggatcgaagtctgcaacccgacttcgtgaagctggattcgctagtaat |
| | | cgcgcatcagccacggcgcggtgaatacgttcccgggccttgtacacaccgcccgtcaagccatgggagccgggggtacct |
| | | gaagtacgtaaccgcaaggatcgtcctagggtaaaactggtgactggggctaagtcgtaacaaggtagccgtaccggaaggt |
| | | gcggctggaacacctcctt |
| Bacteroides_ | SEQ ID | gcatggtttcattattaaaggattccggtaaaggatggggatgcgttccattaggttgttggtgaggtaacggctcaccaagcctt |
| fragilis | NO: 187 | cgatggataggggttctgagaggaaggtcccccacattggaactgagacacggtccaaactcctacgggaggcagcagtga |
| | | ggaatattggtcaatgggcgctagcctgaaccagccaagtagcgtgaaggatgaaggctctatgggtcgtaaacttcttttatat |
| | | aagaataaagtgcagtatgtatactgttttgtatgtattatatgaataaggatcggctaactccgtgccagcagccgcggtaatac |
| | | ggaggatccgagcgttatccggatttattgggtttaaagggagcgtaggtggactggtaagtcagttgtgaaagtttgcggctca |
| | | accgtaaaattgcagttgatactgtcagtcttgagtacagtagaggtgggcggaattcgtggtgtagcggtgaaatgcttagatat |
| | | cacgaagaactccgattgcgaaggcagacactggactgcaactgacactgatgctcgaaagtgtgggtatcaaacaggatta |
| | | gataccctggtagtccacacagtaaacgatgaatactcgctgtttgcgatatacagtaagcggccaagcgaaagcattaagtatt |
| | | ccacctggggagtacgccggcaacggtgaaactcaaaggaattgacgggggcccgcacaagcggaggaacatgtggttta |
| | | attcgatgatacgcgaggaaccttacccgggcttaaattgcagtggaatgatgtggaaacatgtcagtgagcaatcaccgctgt |
| | | gaaggtgctgcatggttgtcgtcagctcgtgccgtgaggtgtcggcttaagtgccataacgagcgcaacccttatctttagttact |
| | | aacaggttatgctgaggactctggagagactgccgtcgtaagatgtgaggaaggtggggatgacgtcaaatcagcacggccc |
| | | ttacgtccggggctacacacgtgttacaatggggggtacagaaggcagctagcgggtgaccgtatgctaatcccaaaagcct |
| | | ctctcagttcggatcgaagtctgcaacccgacttcgtgaagctggattcgctagtaatcgcgcatcagccacggcgcggtgaat |
| | | acgttcccgggccttgtacacaccgcccgtcaagccatgggagccgggggtacctgaagtacgtaaccgcaaggatcgtcct |
| | | agggtaaaactggtgactggggctaagtcgtaacaaggtagccgtaccggaaggtgcggctggaacacctcctt |
| Bacteroides_ | SEQ ID | atgaagagtttgatcctggctcaggatgaacgctagctacaggcttaacacatgcaagtcgaggggcatcaggaagaaagctt |
| fragilis | NO: 188 | gctttctttgctggcgaccggcgcacgggtgagtaacacgtatccaacctgccctttactcggggatagcctttcgaaagaaag |
| | | attaatacccgatagtataatgattccgcatggtttcattattaaaggattccggtaaaggatggggatgcgttccattaggttgttg |
| | | gtgaggtaacggctcaccaagccttcgatggataggggttctgagaggaaggtcccccacattggaactgagacacggtcca |
| | | aactcctacgggaggcagcagtgaggaatattggtcaatgggcgctagcctgaaccagccaagtagcgtgaaggatgaagg |
| | | ctctatgggtcgtaaacttcttttatataagaataaagtgcagtatgtatactgttttgtatgtattatatgaataaggatcggctaactc |
| | | cgtgccagcagccgcggtaatacggaggatccgagcgttatccggatttattgggtttaaagggagcgtaggtggactggtaa |
| | | gtcagttgtgaaagtttgcggctcaaccgtaaaattgcagttgatactgtcagtcttgagtacagtagaggtgggcggaattcgtg |
| | | gtgtagcggtgaaatgcttagatatcacgaagaactccgattgcgaaggcagctcactggactgcaactgacactgatgctcg |
| | | aaagtgtgggtatcaaacaggattagataccctggtagtccacacagtaaacgatgaatactcgctgtttgcgatatacagtaag |
| | | cggccaagcgaaagcattaagtattccacctggggagtacgccggcaacggtgaaactcaaaggaattgacgggggcccg |
| | | cacaagcggaggaacatgtggtttaattcgatgatacgcgaggaaccttacccgggcttaaattgcagtggaatgatgtggaaa |
| | | catgtcagtgagcaatcaccgctgtgaaggtgctgcatggttgtcgtcagctcgtgccgtgaggtgtcggcttaagtgccataa |
| | | cgagcgcaacccttatctttagttactaacaggttatgctgaggactctggagagactgccgtcgtaagatgtgaggaaggtgg |
| | | ggatgacgtcaaatcagcacggcccttacgtccggggctacacacgtgttacaatggggggtacagaaggcagctagcggg |
| | | tgaccgtatgctaatcccaaaagcctctctcagttcggatcgaagtctgcaacccgacttcgtgaagctggattcgctagtaatc |
| | | gcgcatcagccacggcgcggtgaatacgttcccgggccttgtacacaccgcccgtcaagccatgggagccgggggtacctg |
| | | aagtacgtaaccgcaaggatcgtcctagggtaaaactggtgactggggctaagtcgtaacaaggtagccgtaccggaaggtg |
| | | cggctggaacacctcctt |
| Bacteroides_ | SEQ ID | atgaagagtttgatcctggctcaggatgaacgctagctacaggcttaacacatgcaagtcgaggggcatcaggaagaaagctt |
| fragilis | NO: 189 | gctttctttgctggcgaccggcgcacgggtgagtaacacgtatccaacctgccctttactcggggatagcctttcgaaagaaag |
| | | attaatacccgatggcataatgattccgcatggtttcattattaaaggattccggtaaaggatggggatgcgttccattaggttgttg |
| | | gtgaggtaacggcccaccaagccttcgatggataggggttctgagaggaaggtcccccacattggaactgagacacggtcca |
| | | aactcctacgggaggcagcagtgaggaatattggtcaatgggcgctagcctgaaccagccaagtagcgtgaaggatgaagg |
| | | ctctatgggtcgtaaacttcttttatataagaataaagtgcagtatgtatactgttttgtatgtattatatgaataaggatcggctaactc |
| | | cgtgccagcagccgcggtaatacggaggatccgagcgttatccggatttattgggtttaaagggagcgtaggtggactggtaa |
| | | gtcagttgtgaaagtttgcggctcaaccgtaaaattgcagttgatactgtcagtcttgagtacagtagaggtgggcggaattcgtg |
| | | gtgtagcggtgaaatgcttagatatcacgaagaactccgattgcgaaggcagctcactggactgcaactgacactgatgctcg |
| | | aaagtgtgggtatcaaacaggattagataccctggtagtccacacagtaaacgatgaatactcgctgtttgcgatatacagtaag |
| | | cggccaagcgaaagcattaagtattccacctggggagtacgccggcaacggtgaaactcaaaggaattgacgggggcccg |
| | | cacaagcggaggaacatgtggtttaattcgatgatacgcgaggaaccttacccgggcttaaattgcagtggaatgatgtggaaa |
| | | catgtcagtgagcaatcaccgctgtgaaggtgctgcatggttgtcgtcagctcgtgccgtgaggtgtcggcttaagtgccataa |
| | | cgagcgcaacccttatcttcagttactaacaggtcatgctgaggactctggagagactgccgtcgtaagatgtgaggaaggtg |
| | | gggatgacgtcaaatcagcacggcccttacgtccggggctacacacgtgttacaatggggggtacagaaggcagctagcgg |
| | | gtgaccgtatgctaatcccaaaagcctctctcagttcggatcgaagtctgcaacccgacttcgtgaagctggattcgctagtaat |
| | | cgcgcatcagccacggcgcggtgaatacgttcccgggccttgtacacaccgcccgtcaagccatgggagccgggggtacct |
| | | gaagtacgtaaccgcaaggatcgtcctagggtaaaactggtgactggggctaagtcgtaacaaggtagccgtaccggaaggt |
| | | gcggctggaacacctcctt |
| Bacteroides_ | SEQ ID | atgaagagtttgatcctggctcaggatgaacgctagctacaggcttaacacatgcaagtcgaggggcatcaggaagaaagctt |
| fragilis | NO: 190 | gctttctttgctggcgaccggcgcacgggtgagtaacacgtatccaacctgccctttactcggggatagcctttcgaaagaaag |
| | | attaatacccgatggcataatgattccgcatggtttcattattaaaggattccggtaaaggatggggatgcgttccattaggttgttg |
| | | gtgaggtaacggctcaccaagccttcgatggataggggttctgagaggaaggtcccccacattggaactgagacacggtcca |
| | | aactcctacgggaggcagcagtgaggaatattggtcaatgggcgctagcctgaaccagccaagtagcgtgaaggatgaagg |
| | | ctctatgggtcgtaaacttcttttatataagaataaagtgcagtatgtatactgttttgtatgtattatatgaataaggatcggctaactc |
| | | cgtgccagcagccgcggtaatacggaggatccgagcgttatccggatttattgggtttaaagggagcgtaggtggactggtaa |
| | | gtcagttgtgaaagtttgcggctcaaccgtaaaattgcagttgatactgtcagtcttgagtacagtagaggtgggcggaattcgtg |
| | | gtgtagcggtgaaatgcttagatatcacgaagaactccgattgcgaaggcagctcactggactgcaactgacactgatgctcg |
| | | aaagtgtgggtatcaaacaggattagataccctggtagtccacacagtaaacgatgaatactcgctgtttgcgatatacagtaag |
| | | cggccaagcgaaagcattaagtattccacctggggagtacgccggcaacggtgaaactcaaaggaattgacgggggcccg |
| | | cacaagcggaggaacatgtggtttaattcgatgatacgcgaggaaccttacccgggcttaaattgcagtggaatgatgtggaaa |
| | | catgtcagtgagcaatcaccgctgtgaaggtgctgcatggttgtcgtcagctcgtgccgtgaggtgtcggcttaagtgccataa |
| | | cgagcgcaacccttatcttcagttactaacaggtcatgctgaggactctggagagactgccgtcgtaagatgtgaggaaggtg |
| | | gggatgacgtcaaatcagcacggcccttacgtccggggctacacacgtgttacaatggggggtacagaaggcagctagcgg |
| | | gtgaccgtatgctaatcccaaaagcctctctcagttcggatcgaagtctgcaacccgacttcgtgaagctggattcgctagtaat |
| | | cgcgcatcagccacggcgcggtgaatacgttcccgggccttgtacacaccgcccgtcaagccatgggagccgggggtacct |
| | | gaagtacgtaaccgcaaggatcgtcctagggtaaaactggtgactggggctaagtcgtaacaaggtagccgtaccggaaggt |
| | | gcggctggaacacctcctt |
| Bacteroides_ | SEQ ID | atgaagagtttgatcctggctcaggatgaacgctagctacaggcttaacacatgcaagtcgaggggcatcaggaagaaagctt |
| fragilis | NO: 191 | gctttctttgctggcgaccggcgcacgggtgagtaacacgtatccaacctgccctttactcggggatagcctttcgaaagaaag |
| | | attaatacccgatggcataatgattccgcatggtttcattattaaaggattccggtaaaggatggggatgcgttccattaggttgttg |
| | | gtgaggtaacggcccaccaagccttcgatggataggggttctgagaggaaggtcccccacattggaactgagacacggtcca |
| | | aactcctacgggaggcagcagtgaggaatattggtcaatgggcgctagcctgaaccagccaagtagcgtgaaggatgaagg |
| | | ctctatgggtcgtaaacttcttttatataagaataaagtgcagtatgtatactgttttgtatgtattatatgaataaggatcggctaactc |
| | | cgtgccagcagccgcggtaatacggaggatccgagcgttatccggatttattgggtttaaagggagcgtaggtggactggtaa |
| | | gtcagttgtgaaagtttgcggctcaaccgtaaaattgcagttgatactgtcagtcttgagtacagtagaggtgggcggaattcgtg |
| | | gtgtagcggtgaaatgcttagatatcacgaagaactccgattgcgaaggcagctcactggactgcaactgacactgatgctcg |
| | | aaagtgtgggtatcaaacaggattagataccctggtagtccacacagtaaacgatgaatactcgctgtttgcgatatacagtaag |
| | | cggccaagcgaaagcattaagtattccacctggggagtacgccggcaacggtgaaactcaaaggaattgacgggggcccg |
| | | cacaagcggaggaacatgtggtttaattcgatgatacgcgaggaaccttacccgggcttaaattgcagtggaatgatgtggaaa |
| | | catgtcagtgagcaatcaccgctgtgaaggtgctgcatggttgtcgtcagctcgtgccgtgaggtgtcggcttaagtgccataa |
| | | cgagcgcaacccttatcttcagttactaacaggtcatgctgaggactctggagagactgccgtcgtaagatgtgaggaaggtg |
| | | gggatgacgtcaaatcagcacggcccttacgtccggggctacacacgtgttacaatggggggtacagaaggcagctagcgg |
| | | gtgaccgtatgctaatcccaaaagcctctctcagttcggatcgaagtctgcaacccgacttcgtgaagctggattcgctagtaat |
| | | cgcgcatcagccacggcgcggtgaatacgttcccgggccttgtacacaccgcccgtcaagccatgggagccgggggtacct |
| | | gaagtacgtaaccgcaaggatcgtcctagggtaaaactggtgactggggctaagtcgtaacaaggtagccgtaccggaaggt |
| | | gcggctggaacacctcctt |
| Bacteroides_ | SEQ ID | actgagacacggtccaaactcctacgggaggcagcagtgaggaatattggtcaatggacgagagtctgaaccagccaagta |
| uniformis | NO: 192 | gcgtgaaggatgactgccctatgggttgtaaacttcttttatacgggaataaagtgaggcacgtgtgcctttttgtatgtaccgtat |
| | | gaataaggatcggctaactccgtgccagcagccgcggtaatacggaggatccgagcgttatccggatttattgggtttaaagg |
| | | gagcgtaggcggacgcttaagtcagttgtgaaagtttgcggctcaaccgtaaaattgcagttgatactgggtgtcttgagtacag |
| | | tagaggcaggcggaattcgtggtgtagcggtgaaatgcttagatatcacgaagaactccgattgcgaaggcagcttgctggac |
| | | tgtaactgacgctgatgctcgaaagtgtgggtatcaaacaggattagataccctggtagtccacacagtaaacgatgaatactc |
| | | gctgtttgcgatatacagtaagcggccaagcgaaagcgttaagtattccacctggggagtacgccggcaacggtgaaactcaa |
| | | aggaattgacgggggcccgcacaagcggaggaacatgtggtttaattcgatgatacgcgaggaaccttacccgggcttgaatt |
| | | gcaactgaatgatgtggagacatgtcagccgcaaggcagttgtgaaggtgctgcatggttgtcgtcagctcgtgccgtgaggt |
| | | gtcggcttaagtgccataacgagcgcaacccttatcgatagttaccatcaggttatgctggggactctgtcgagactgccgtcgt |
| | | aagatgtgaggaaggtggggatgacgtcaaatcagcacggcccttacgtccggggctacacacgtgttacaatggggggtac |
| | | agaaggcagctacacggcgacgtgatgctaatcccgaaagcctctctcagttcggattggagtctgcaacccgactccatgaa |
| | | gctggattcgctagtaatcgcgcatcagccacggcgcggtgaatacgttcccgggccttgtacacaccgcccgtcaagccatg |
| | | aaagccgggggtacctgaagtgcgtaaccgcaaggagcgccctagggtaaaactggtgattggggctaagtcgtaacaagg |
| | | tagccgtaccggaaggtgcggctggaacacctcctt |
| Bacteroides_ | SEQ ID | atgaagagtttgatcctggctcaggatgaacgctagctacaggcttaacacatgcaagtcgaggggcagcatgaacttagcttg |
| uniformis | NO: 193 | ctaagtttgatggcgaccggcgcacgggtgagtaacacgtatccaacctgccgatgactcggggatagcctttcgaaagaaa |
| | | gattaatacccgatggcatagttcttccgcatggtggaactattaaagaatttcggtcatcgatggggatgcgttccattaggttgtt |
| | | ggcggggtaacggcccaccaagccttcgatggataggggttctgagaggaaggtcccccacattggaactgagacacggtc |
| | | caaactcctacgggaggcagcagtgaggaatattggtcaatggacgagagtctgaaccagccaagtagcgtgaaggatgact |
| | | gccctatgggttgtaaacttcttttatacgggaataaagtgaggcacgtgtgcctttttgtatgtaccgtatgaataaggatcggcta |
| | | actccgtgccagcagccgcggtaatacggaggatccgagcgttatccggatttattgggtttaaagggagcgtaggcggacg |
| | | cttaagtcagttgtgaaagtttgcggctcaaccgtaaaattgcagttgatactgggtgtcttgagtacagtagaggcaggcggaa |
| | | ttcgtggtgtagcggtgaaatgcttagatatcacgaagaactccgattgcgaaggcagcttgctggactgtaactgacgctgatg |
| | | ctcgaaagtgtgggtatcaaacaggattagataccctggtagtccacacagtaaacgatgaatactcgctgtttgcgatatacag |
| | | taagcggccaagcgaaagcgttaagtattccacctggggagtacgccggcaacggtgaaactcaaaggaattgacgggggc |
| | | ccgcacaagcggaggaacatgtggtttaattcgatgatacgcgaggaaccttacccgggcttgaattgcaactgaatgatgtgg |
| | | agacatgtcagccgcaaggcagttgtgaaggtgctgcatggttgtcgtcagctcgtgccgtgaggtgtcggcttaagtgccata |
| | | acgagcgcaacccttatcgatagttaccatcaggttatgctggggactctgtcgagactgccgtcgtaagatgtgaggaaggtg |
| | | gggatgacgtcaaatcagcacggcccttacgtccggggctacacacgtgttacaatggggggtacagaaggcagctacacg |
| | | gcgacgtgatgctaatcccgaaagcctctctcagttcggattggagtctgcaacccgactccatgaagctggattcgctagtaat |
| | | cgcgcatcagccacggcgcggtgaatacgttcccgggccttgtacacaccgcccgtcaagccatgaaagccgggggtacct |
| | | gaagtgcgtaaccgcaaggagcgccctagggtaaaactggtgattggggctaagtcgtaacaaggtagccgtaccggaagg |
| | | tgcggctggaacacctcctt |
| Bacteroides_ | SEQ ID | actgagacacggtccaaactcctacgggaggcagcagtgaggaatattggtcaatggacgagagtctgaaccagccaagta |
| uniformis | NO: 194 | gcgtgaaggatgactgccctatgggttgtaaacttcttttatacgggaataaagtgaggcacgtgtgcctttttgtatgtaccgtat |
| | | gaataaggatcggctaactccgtgccagcagccgcggtaatacggaggatccgagcgttatccggatttattgggtttaaagg |
| | | gagcgtaggcggacgcttaagtcagttgtgaaagtttgcggctcaaccgtaaaattgcagttgatactgggtgtcttgagtacag |
| | | tagaggcaggcggaattcgtggtgtagcggtgaaatgcttagatatcacgaagaactccgattgcgaaggcagcttgctggac |
| | | tgtaactgacgctgatgctcgaaagtgtgggtatcaaacaggattagataccctggtagtccacacagtaaacgatgaatactc |
| | | gctgtttgcgatatacagtaagcggccaagcgaaagcgttaagtattccacctggggagtacgccggcaacggtgaaactcaa |
| | | aggaattgacgggggcccgcacaagcggaggaacatgtggtttaattcgatgatacgcgaggaaccttacccgggcttgaatt |
| | | gcaactgaatgatgtggagacatgtcagccgcaaggcagttgtgaaggtgctgcatggttgtcgtcagctcgtgccgtgaggt |
| | | gtcggcttaagtgccataacgagcgcaacccttatcgatagttaccatcaggttatgctggggactctgtcgagactgccgtcgt |
| | | aagatgtgaggaaggtggggatgacgtcaaatcagcacggcccttacgtccggggctacacacgtgttacaatggggggtac |
| | | agaaggcagctacacggcgacgtgatgctaatcccgaaagcctctctcagttcggattggagtctgcaacccgactccatgaa |
| | | gctggattcgctagtaatcgcgcatcagccacggcgcggtgaatacgttcccgggccttgtacacaccgcccgtcaagccatg |
| | | aaagccgggggtacctgaagtgcgtaaccgcaaggagcgccctagggtaaaactggtgattggggctaagtcgtaacaagg |
| | | tagccgtaccggaaggtgcggctggaacacctcctt |
| Bacteroides_ | SEQ ID | atgaagagtttgatcctggctcaggatgaacgctagctacaggcttaacacatgcaagtcgaggggcagcatgaacttagcttg |
| uniformis | NO: 195 | ctaagtttgatggcgaccggcgcacgggtgagtaacacgtatccaacctgccgatgactcggggatagcctttcgaaagaaa |
| | | gattaatacccgatggcatagttcttccgcatggtagaactattaaagaatttcggtcatcgatggggatgcgttccattaggttgtt |
| | | ggcggggtaacggcccaccaagccttcgatggataggggttctgagaggaaggtcccccacattggaactgagacacggtc |
| | | caaactcctacgggaggcagcagtgaggaatattggtcaatggacgagagtctgaaccagccaagtagcgtgaaggatgact |
| | | gccctatgggttgtaaacttcttttatacgggaataaagtgaggcacgtgtgcctttttgtatgtaccgtatgaataaggatcggcta |
| | | actccgtgccagcagccgcggtaatacggaggatccgagcgttatccggatttattgggtttaaagggagcgtaggcggacg |
| | | cttaagtcagttgtgaaagtttgcggctcaaccgtaaaattgcagttgatactgggtgtcttgagtacagtagaggcaggcggaa |
| | | ttcgtggtgtagcggtgaaatgcttagatatcacgaagaactccgattgcgaaggcagcttgctggactgtaactgacgctgatg |
| | | ctcgaaagtgtgggtatcaaacaggattagataccctggtagtccacacagtaaacgatgaatactcgctgtttgcgatatacag |
| | | taagcggccaagcgaaagcgttaagtattccacctggggagtacgccggcaacggtgaaactcaaaggaattgacgggggc |
| | | ccgcacaagcggaggaacatgtggtttaattcgatgatacgcgaggaaccttacccgggcttgaattgcaactgaatgatgtgg |
| | | agacatgtcagccgcaaggcagttgtgaaggtgctgcatggttgtcgtcagctcgtgccgtgaggtgtcggcttaagtgccata |
| | | acgagcgcaacccttatcgatagttaccatcaggttatgctggggactctgtcgagactgccgtcgtaagatgtgaggaaggtg |
| | | gggatgacgtcaaatcagcacggcccttacgtccggggctacacacgtgttacaatggggggtacagaaggcagctacacg |
| | | gcgacgtgatgctaatcccgaaagcctctctcagttcggattggagtctgcaacccgactccatgaagctggattcgctagtaat |
| | | cgcgcatcagccacggcgcggtgaatacgttcccgggccttgtacacaccgcccgtcaagccatgaaagccgggggtacct |
| | | gaagtgcgtaaccgcaaggagcgccctagggtaaaactggtgattggggctaagtcgtaacaaggtagccgtaccggaagg |
| | | tgcggctggaacacctcctt |
| Bacteroides_ | SEQ ID | atgaagagtttgatcctggctcaggatgaacgctagctacaggcttaacacatgcaagtcgaggggcagcatgaacttagcttg |
| uniformis | NO: 196 | ctaagtttgatggcgaccggcgcacgggtgagtaacacgtatccaacctgccgatgactcggggatagcctttcgaaagaaa |
| | | gattaatacccgatggcatagttcttccgcatggtggaactattaaagaatttcggtcatcgatggggatgcgttccattaggttgtt |
| | | ggcggggtaacggcccaccaagccttcgatggataggggttctgagaggaaggtcccccacattggaactgagacacggtc |
| | | caaactcctacgggaggcagcagtgaggaatattggtcaatggacgagagtctgaaccagccaagtagcgtgaaggatgact |
| | | gccctatgggttgtaaacttcttttatacgggaataaagtgaggcacgtgtgcctttttgtatgtaccgtatgaataaggatcggcta |
| | | actccgtgccagcagccgcggtaatacggaggatccgagcgttatccggatttattgggtttaaagggagcgtaggcggacg |
| | | cttaagtcagttgtgaaagtttgcggctcaaccgtaaaattgcagttgatactgggtgtcttgagtacagtagaggcaggcggaa |
| | | ttcgtggtgtagcggtgaaatgcttagatatcacgaagaactccgattgcgaaggcagcttgctggactgtaactgacgctgatg |
| | | ctcgaaagtgtgggtatcaaacaggattagataccctggtagtccacacagtaaacgatgaatactcgctgtttgcgatatacag |
| | | taagcggccaagcgaaagcgttaagtattccacctggggagtacgccggcaacggtgaaactcaaaggaattgacgggggc |
| | | ccgcacaagcggaggaacatgtggtttaattcgatgatacgcgaggaaccttacccgggcttgaattgcaactgaatgatgtgg |
| | | agacatgtcagccgcaaggcagttgtgaaggtgctgcatggttgtcgtcagctcgtgccgtgaggtgtcggcttaagtgccata |
| | | acgagcgcaacccttatcgatagttaccatcaggttatgctggggactctgtcgagactgccgtcgtaagatgtgaggaaggtg |
| | | gggatgacgtcaaatcagcacggcccttacgtccggggctacacacgtgttacaatggggggtacagaaggcagctacacg |
| | | gcgacgtgatgctaatcccgaaagcctctctcagttcggattggagtctgcaacccgactccatgaagctggattcgctagtaat |
| | | cgcgcatcagccacggcgcggtgaatacgttcccgggccttgtacacaccgcccgtcaagccatgaaagccgggggtacct |
| | | gaagtgcgtaaccgcaaggagcgccctagggtaaaactggtgattggggctaagtcgtaacaaggtagccgtaccggaagg |
| | | tgcggctggaacacctcctt |
| Bacteroides_ | SEQ ID | atgaagagtttgatcctggctcaggatgaacgctagctacaggcttaacacatgcaagtcgaggggcagcatgaacttagcttg |
| uniformis | NO: 197 | ctaagtttgatggcgaccggcgcacgggtgagtaacacgtatccaacctgccgatgactcggggatagcctttcgaaagaaa |
| | | gattaatacccgatggcatagttcttccgcatggtagaactattaaagaatttcggtcatcgatggggatgcgttccattaggttgtt |
| | | ggcggggtaacggcccaccaagccttcgatggataggggttctgagaggaaggtcccccacattggaactgagacacggtc |
| | | caaactcctacgggaggcagcagtgaggaatattggtcaatggacgagagtctgaaccagccaagtagcgtgaaggatgact |
| | | gccctatgggttgtaaacttcttttatacgggaataaagtgaggcacgtgtgcctttttgtatgtaccgtatgaataaggatcggcta |
| | | actccgtgccagcagccgcggtaatacggaggatccgagcgttatccggatttattgggtttaaagggagcgtaggcggacg |
| | | cttaagtcagttgtgaaagtttgcggctcaaccgtaaaattgcagttgatactgggtgtcttgagtacagtagaggcaggcggaa |
| | | ttcgtggtgtagcggtgaaatgcttagatatcacgaagaactccgattgcgaaggcagcttgctggactgtaactgacgctgatg |
| | | ctcgaaagtgtgggtatcaaacaggattagataccctggtagtccacacagtaaacgatgaatactcgctgtttgcgatatacag |
| | | taagcggccaagcgaaagcgttaagtattccacctggggagtacgccggcaacggtgaaactcaaaggaattgacgggggc |
| | | ccgcacaagcggaggaacatgtggtttaattcgatgatacgcgaggaaccttacccgggcttgaattgcaactgaatgatgtgg |
| | | agacatgtcagccgcaaggcagttgtgaaggtgctgcatggttgtcgtcagctcgtgccgtgaggtgtcggcttaagtgccata |
| | | acgagcgcaacccttatcgatagttaccatcaggttatgctggggactctgtcgagactgccgtcgtaagatgtgaggaaggtg |
| | | gggatgacgtcaaatcagcacggcccttacgtccggggctacacacgtgttacaatggggggtacagaaggcagctacacg |
| | | gcgacgtgatgctaatcccgaaagcctctctcagttcggattggagtctgcaacccgactccatgaagctggattcgctagtaat |
| | | cgcgcatcagccacggcgcggtgaatacgttcccgggccttgtacacaccgcccgtcaagccatgaaagccgggggtacct |
| | | gaagtgcgtaaccgcaaggagcgccctagggtaaaactggtgattggggctaagtcgtaacaaggtagccgtaccggaagg |
| | | tgcggctggaacacctcctt |
| Bacteroides_ | SEQ ID | atgaagagtttgatcctggctcaggatgaacgctagctacaggcttaacacatgcaagtcgaggggcagcatgaacttagcttg |
| uniformis | NO: 198 | ctaagtttgatggcgaccggcgcacgggtgagtaacacgtatccaacctgccgatgactcggggatagcctttcgaaagaaa |
| | | gattaatacccgatggcatagttcttccgcatggtagaactattaaagaatttcggtcatcgatggggatgcgttccattaggttgtt |
| | | ggcggggtaacggcccaccaagccttcgatggataggggttctgagaggaaggtcccccacattggaactgagacacggtc |
| | | caaactcctacgggaggcagcagtgaggaatattggtcaatggacgagagtctgaaccagccaagtagcgtgaaggatgact |
| | | gccctatgggttgtaaacttcttttatacgggaataaagtgaggcacgtgtgcctttttgtatgtaccgtatgaataaggatcggcta |
| | | actccgtgccagcagccgcggtaatacggaggatccgagcgttatccggatttattgggtttaaagggagcgtaggcggacg |
| | | cttaagtcagttgtgaaagtttgcggctcaaccgtaaaattgcagttgatactgggtgtcttgagtacagtagaggcaggcggaa |
| | | ttcgtggtgtagcggtgaaatgcttagatatcacgaagaactccgattgcgaaggcagcttgctggactgtaactgacgctgatg |
| | | ctcgaaagtgtgggtatcaaacaggattagataccctggtagtccacacagtaaacgatgaatactcgctgtttgcgatatacag |
| | | taagcggccaagcgaaagcgttaagtattccacctggggagtacgccggcaacggtgaaactcaaaggaattgacgggggc |
| | | ccgcacaagcggaggaacatgtggtttaattcgatgatacgcgaggaaccttacccgggcttgaattgcaactgaatgatgtgg |
| | | agacatgtcagccgcaaggcagttgtgaaggtgctgcatgttgtcgtcagctcgtgccgtgaggtgtcggcttaagtgccata |
| | | acgagcgcaacccttatcgatagttaccatcaggttatgctggggactctgtcgagactgccgtcgtaagatgtgaggaaggtg |
| | | gggatgacgtcaaatcagcacggcccttacgtccggggctacacacgtgttacaatggggggtacagaaggcagctacacg |
| | | gcgacgtgatgctaatcccgaaagcctctctcagttcggattggagtctgcaacccgactccatgaagctggattcgctagtaat |
| | | cgcgcatcagccacggcgcggtgaatacgttcccgggccttgtacacaccgcccgtcaagccatgaaagccgggggtacct |
| | | gaagtgcgtaaccgcaaggagcgccctagggtaaaactggtgattggggctaagtcgtaacaaggtagccgtaccggaagg |
| | | tgcggctggaacacctcctt |
| Bacteroides_ | SEQ ID | tttgcatgtactttatgaataaggatcggctaactccgtgccagcagccgcggtaatacggaggatccgagcgttatccggattt |
| vulgatus | NO: 199 | attgggtttaaagggagcgtagatggatgtttaagtcagttgtgaaagtttgcggctcaaccgtaaaattgcagttgatactggat |
| | | atcttgagtgcagttgaggcaggcggaattcgtggtgtagcggtgaaatgcttagatatcacgaagaactccgattgcgaaggc |
| | | agcctgctaagctgcaactgacattgaggctcgaaagtgtgggtatcaaacaggattagataccctggtagtccacacggtaaa |
| | | cgatgaatactcgctgtttgcgatatacggcaagcggccaagcgaaagcgttaagtattccacctggggagtacgccggcaac |
| | | ggtgaaactcaaaggaattgacgggggcccgcacaagcggaggaacatgtggtttaattcgatgatacgcgaggaaccttac |
| | | ccgggcttaaattgcagatgaattacggtgaaagccgtaagccgcaaggcatctgtgaaggtgctgcatggttgtcgtcagctc |
| | | gtgccgtgaggtgtcggcttaagtgccataacgagcgcaacccttgttgtcagttactaacaggttctgctgaggactctgacaa |
| | | gactgccatcgtaagatgtgaggaaggtggggatgacgtcaaatcagcacggcccttacgtccggggctacacacgtgttac |
| | | aatggggggtacagagggccgctaccacgcgagtggatgccaatcccaaaaacctctctcagttcggactggagtctgcaac |
| | | ccgactccacgaagctggattcgctagtaatcgcgcatcagccacggcgcggtgaatacgttcccgggccttgtacacaccgc |
| | | ccgtcaagccatgggagccgggggtacctgaagtgcgtaaccgcgaggagcgccctagggtaaaactggtgactggggct |
| | | aagtcgtaacaaggtagccgtaccggaaggtgcggctggaacacctccttt |
| Bacteroides_ | SEQ ID | atgaagagtttgatcctggctcaggatgaacgctagctacaggcttaacacatgcaagtcgaggggcagcatggtcttagcttg |
| vulgatus | NO: 200 | ctaaggccgatggcgaccggcgcacgggtgagtaacacgtatccaacctgccgtctactcttggacagccttctgaaaggaa |
| | | gattaatacaagatggcatcatgagttcacatgttcacatgattaaaggtattccggtagacgatggggatgcgttccattagata |
| | | gtaggcggggtaacggcccacctagtcttcgatggataggggttctgagaggaaggtcccccacattggaactgagacacgg |
| | | tccaaactcctacgggaggcagcagtgaggaatattggtcaatgggcgagagcctgaaccagccaagtagcgtgaaggatg |
| | | actgccctatgggttgtaaacttcttttataaaggaataaagtcgggtatggatacccgtttgcatgtactttatgaataaggatcgg |
| | | ctaactccgtgccagcagccgcggtaatacggaggatccgagcgttatccggatttattgggtttaaagggagcgtagatggat |
| | | gtttaagtcagttgtgaaagtttgcggctcaaccgtaaaattgcagttgatactggatatcttgagtgcagttgaggcaggcggaa |
| | | ttcgtggtgtagcggtgaaatgcttagatatcacgaagaactccgattgcgaaggcagcctgctaagctgcaactgacattgag |
| | | gctcgaaagtgtgggtatcaaacaggattagataccctggtagtccacacggtaaacgatgaatactcgctgtttgcgatatacg |
| | | gcaagcggccaagcgaaagcgttaagtattccacctggggagtacgccggcaacggtgaaactcaaaggaattgacgggg |
| | | gcccgcacaagcggaggaacatgtggtttaattcgatgatacgcgaggaaccttacccgggcttaaattgcagatgaattacg |
| | | gtgaaagccgtaagccgcaaggcatctgtgaaggtgctgcatggttgtcgtcagctcgtgccgtgaggtgtcggcttaagtgc |
| | | cataacgagcgcaacccttgttgtcagttactaacaggttctgctgaggactctgacaagactgccatcgtaagatgtgaggaa |
| | | ggtggggatgacgtcaaatcagcacggcccttacgtccggggctacacacgtgttacaatggggggtacagagggccgcta |
| | | ccacgcgagtggatgccaatcccaaaaacctctctcagttcggactggagtctgcaacccgactccacgaagctggattcgct |
| | | agtaatcgcgcatcagccacggcgcggtgaatacgttcccgggccttgtacacaccgcccgtcaagccatgggagccgggg |
| | | gtacctgaagtgcgtaaccgcgaggagcgccctagggtaaaactggtgactggggctaagtcgtaacaaggtagccgtacc |
| | | ggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| vulgatus | NO: 201 | ctaaggccgatggcgaccggcgcacgggtgagtaacacgtatccaacctgccgtctactcttggacagccttctgaaaggaa |
| | | gattaatacaagatggcatcatgagtccgcatgttcacatgattaaaggtattccggtaggcgatggggatgcgttccattagata |
| | | gtaggcggggtaacggcccacctagtcttcgatggataggggttctgagaggaaggtcccccacattggaactgagacacgg |
| | | tccaaactcctacgggaggcagcagtgaggaatattggtcaatgggcgagagcctgaaccagccaagtagcgtgaaggatg |
| | | actgccctatgggttgtaaacttcttttataaaggaataaagtcgggtatgtatacccgtttgcatgtactttatgaataaggatcgg |
| | | ctaactccgtgccagcagccgcggtaatacggaggatccgagcgttatccggatttattgggtttaaagggagcgtagatggat |
| | | gtttaagtcagttgtgaaagtttgcggctcaaccgtaaaattgcagttgatactggatatcttgagtgcagttgaggcaggcggaa |
| | | ttcgtggtgtagcggtgaaatgcttagatatcacgaagaactccgattgcgaaggcagcctgctaagctgcaactgacattgag |
| | | gctcgaaagtgtgggtatcaaacaggattagataccctggtagtccacacggtaaacgatgaatactcgctgtttgcgatataca |
| | | gcaagcggccaagcgaaagcgttaagtattccacctggggagtacgccggcaacggtgaaactcaaaggaattgacgggg |
| | | gcccgcacaagcggaggaacatgtggtttaattcgatgatacgcgaggaaccttacccgggcttaaattgcagatgaattacg |
| | | gtgaaagccgtaagccgcaaggcatctgtgaaggtgctgcatggttgtcgtcagctcgtgccgtgaggtgtcggcttaagtgc |
| | | cataacgagcgcaacccttgttgtcagttactaacaggttctgctgaggactctgacaagactgccatcgtaagatgtgaggaa |
| | | ggtggggatgacgtcaaatcagcacggcccttacgtccggggctacacacgtgttacaatggggggtacagagggccgcta |
| | | ccacgcgagtggatgccaatcccaaaaacctctctcagttcggactggagtctgcaacccgactccacgaagctggattcgct |
| | | agtaatcgcgcatcagccacggcgcggtgaatacgttcccgggccttgtacacaccgcccgtcaagccatgggagccgggg |
| | | gtacctgaagtgcgtaaccgcgaggagcgccctagggtaaaactggtgactggggctaagtcgtaacaaggtagccgtacc |
| | | ggaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| vulgatus | NO: 202 | ctaaggccgatggcgaccggcgcacgggtgagtaacacgtatccaacctgccgtctactcttggacagccttctgaaaggaa |
| | | gattaatacaagatggcatcatgagtccgcatgttcacatgattaaaggtattccggtagacgatggggatgcgttccattagata |
| | | gtaggcggggtaacggcccacctagtcttcgatggataggggttctgagaggaaggtcccccacattggaactgagacacgg |
| | | tccaaactcctacgggaggcagcagtgaggaatattggtcaatgggcgcaggcctgaaccagccaagtagcgtgaaggatg |
| | | actgccctatgggttgtaaacttcttttataaaggaataaagtcgggtatgcatacccgtttgcatgtactttatgaataaggatcgg |
| | | ctaactccgtgccagcagccgcggtaatacggaggatccgagcgttatccggatttattgggtttaaagggagcgtagatggat |
| | | gtttaagtcagttgtgaaagtttgcggctcaaccgtaaaattgcagttgatactggatatcttgagtgcagttgaggcaggcggaa |
| | | ttcgtggtgtagcggtgaaatgcttagatatcacgaagaactccgattgcgaaggcagcctgctaagctgcaactgacattgag |
| | | gctcgaaagtgtgggtatcaaacaggattagataccctggtagtccacacggtaaacgatgaatactcgctgtttgcgatataca |
| | | gcaagcggccaagcgaaagcgttaagtattccacctggggagtacgccggcaacggtgaaactcaaaggaattgacgggg |
| | | gcccgcacaagcggaggaacatgtggtttaattcgatgatacgcgaggaaccttacccgggcttaaattgcagatgaattacg |
| | | gtgaaagccgtaagccgcaaggcatctgtgaaggtgctgcatggttgtcgtcagctcgtgccgtgaggtgtcggcttaagtgc |
| | | cataacgagcgcaacccttgttgtcagttactaacaggttccgctgaggactctgacaagactgccatcgtaagatgtgaggaa |
| | | ggtggggatgacgtcaaatcagcacggcccttacgtccggggctacacacgtgttacaatggggggtacagagggtcgctac |
| | | cacgcgagtggatgccaatccccaaaacctctctcagttcggactggagtctgcaacccgactccacgaagctggattcgcta |
| | | gtaatcgcgcatcagccacggcgcggtgaatacgttcccgggccttgtacacaccgcccgtcaagccatgggagccggggg |
| | | tacctgaagtgcgtaaccgcgaggagcgccctagggtaaaactggtgactggggctaagtcgtaacaaggtagccgtaccg |
| | | gaaggtgcggctggaacacctcctt |
| Bacteroides_ | SEQ ID | atgaagagtttgatcctggctcaggatgaacgctagctacaggcttaacacatgcaagtcgaggggcagcatggtcttagcttg |
| vulgatus | NO: 203 | ctaaggccgatggcgaccggcgcacgggtgagtaacacgtatccaacctgccgtctactcttggacagccttctgaaaggaa |
| | | gattaatacaagatggcatcatgagtccgcatgttcacatgattaaaggtattccggtagacgatggggatgcgttccattagata |
| | | gtaggcggggtaacggcccacctagtcttcgatggataggggttctgagaggaaggtcccccacattggaactgagacacgg |
| | | tccaaactcctacgggaggcagcagtgaggaatattggtcaatgggcgagagcctgaaccagccaagtagcgtgaaggatg |
| | | actgccctatgggttgtaaacttcttttataaaggaataaagtcgggtatggatacccgtttgcatgtactttatgaataaggatcgg |
| | | ctaactccgtgccagcagccgcggtaatacggaggatccgagcgttatccggatttattgggtttaaagggagcgtagatggat |
| | | gtttaagtcagttgtgaaagtttgcggctcaaccgtaaaattgcagttgatactggatatcttgagtgcagttgaggcaggcggaa |
| | | ttcgtggtgtagcggtgaaatgcttagatatcacgaagaactccgattgcgaaggcagcctgctaagctgcaactgacattgag |
| | | gctcgaaagtgtgggtatcaaacaggattagataccctggtagtccacacggtaaacgatgaatactcgctgtttgcgatataca |
| | | gcaagcggccaagcgaaagcgttaagtattccacctggggagtacgccggcaacggtgaaactcaaaggaattgacgggg |
| | | gcccgcacaagcggaggaacatgtggtttaattcgatgatacgcgaggaaccttacccgggcttaaattgcagatgaattacg |
| | | gtgaaagccgtaagccgcaaggcatctgtgaaggtgctgcatggttgtcgtcagctcgtgccgtgaggtgtcggcttaagtgc |
| | | cataacgagcgcaacccttgagtcagttactaacaggttctgctgaggactctgacaagactgccatcgtaagatgtgaggaa |
| | | ggtggggatgacgtcaaatcagcacggcccttacgtccggggctacacacgtgttacaatggggggtacagagggccgcta |
| | | ccacgcgagtggatgccaatcccaaaaacctctctcagttcggactggagtctgcaacccgactccacgaagctggattcgct |
| | | agtaatcgcgcatcagccacggcgcggtgaatacgttcccgggccttgtacacaccgcccgtcaagccatgggagccgggg |
| | | gtacctgaagtgcgtaaccgcgaggagcgccctagggtaaaactggtgactggggctaagtcgtaacaaggtagccgtacc |
| | | ggaaggtgcggctggaacacctcctt |
| Bacteroides_ | SEQ ID | ccgtttgcatgtactttatgaataaggatcggctaactccgtgccagcagccgcggtaatacggaggatccgagcgttatccgg |
| vulgatus | NO: 204 | atttattgggtttaaagggagcgtagatggatgtttaagtcagttgtgaaagtttgcggctcaaccgtaaaattgcagttgatactg |
| | | gatatcttgagtgcagttgaggcaggcggaattcgtggtgtagcggtgaaatgcttagatatcacgaagaactccgattgcgaa |
| | | ggcagcctgctaagctgcaactgacattgaggctcgaaagtgtgggtatcaaacaggattagataccctggtagtccacacgg |
| | | taaacgatgaatactcgctgtttgcgatatacagcaagcggccaagcgaaagcgttaagtattccacctggggagtacgccgg |
| | | caacggtgaaactcaaaggaattgacgggggcccgcacaagcggaggaacatgtggtttaattcgatgatacgcgaggaac |
| | | cttacccgggcttaaattgcagatgaattacggtgaaagccgtaagccgcaaggcatctgtgaaggtgctgcatggttgtcgtc |
| | | agctcgtgccgtgaggtgtcggcttaagtgccataacgagcgcaacccttgttgtcagttactaacaggttctgctgaggactct |
| | | gacaagactgccatcgtaagatgtgaggaaggtggggatgacgtcaaatcagcacggcccttacgtccggggctacacacgt |
| | | gttacaatggggggtacagagggccgctaccacgcgagtggatgccaatccccaaaacctctctcagttcggactggagtct |
| | | gcaacccgactccacgaagctggattcgctagtaatcgcgcatcagccacggcgcggtgaatacgttcccgggccttgtacac |
| | | accgcccgtcaagccatgggagccgggggtacctgaagtgcgtaaccgcgaggagcgccctagggtaaaactggtgactg |
| | | gggctaagtcgtaacaaggtagccgtaccggaaggtgcggctggaacacctccttt |
| Eubacterium_ | SEQ ID | atggagagtttgatcctggctcaggatgaacgctggcggcatgcctaatacatgcaagtcgaacgaagtctttaggaagcttgc |
| sp_3_1_31 | NO: 205 | ttccaaagagacttagtggcgaacgggtgagtaacacgtaggtaacctgcccatgtgcccgggataactgctggaaacggta |
| | | gctaaaaccggataggtatgagggaggcatcacctcatattaaagcaccttcgggtgtgaacatggatggacctgcggcgcat |
| | | tagctggttggtgaggtaacggcccaccaaggcgatgatgcgtagccgacctgagagggtgaacggccacattgggactga |
| | | gacacggcccaaactcctacgggaggcagcagtagggaattttcgtcaatggggggaaccctgaacgagcaatgccgcgtg |
| | | tgtgaagaaggtcttcggatcgtaaagcactgttgtaagtgaagaatgccacatagaggaaatgctatgtgggtgacggtagct |
| | | taccagaaagccacggctaactacgtgccagcagccgcggtaatacgtaggtggcaagcgttatccggaatcattgggcgta |
| | | aagggtgcgtaggtggcacgataagtctgaagtaaaaggcaacagctcaactgttgtatgctttggaaactgtcgagctagagt |
| | | gcagaagagggcgatggaattccatgtgtagcggtaaaatgcgtagatatatggaggaacaccagtggcgaaggcggtcgc |
| | | ctggtctgtaactgacactgatgcacgaaagcgtggggagcaaataggattagataccctagtagtccacgccgtaaacgatg |
| | | agaactaagtgttggagagattcagtgctgcagttaacgcaataagttctccgcctggggagtatgcacgcaagtgtgaaactc |
| | | aaaggaattgacgggggcccgcacaagcggtggagtatgtggtttaattcgaagcaacgcgaagaaccttaccaggccttga |
| | | catggatataaatgttctagagatagaaagatagctatatatcacacaggtggtgcatggttgtcgtcagctcgtgtcgtgagatg |
| | | ttgggttaagtcccgcaacgagcgcaacccttgtcttctgttaccagcattaggttggggactcaggagagactgccggtgaca |
| | | aaccggaggaaggtggggatgacgtcaaatcatcatgccccttatggcctgggctacacacgtactacaatggcgcctacaaa |
| | | gagcagcgacaccgcgaggtggagcgaatctcataaagggcgtctcagttcggattgaagtctgcaactcgacttcatgaagt |
| | | cggaatcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagtcgtaa |
| | | caaggtatccctacgggaacgtggggatggatcacctccttt |
| Eubacterium_ | SEQ ID | atggagagtttgatcctggctcaggatgaacgctggcggcatgcctaatacatgcaagtcgaacgaagtctttaggaagcttgc |
| sp_3_1_31 | NO: 206 | ttccaaagagacttagtggcgaacgggtgagtaacacgtaggtaacctgcccatgtgcccgggataactgctggaaacggta |
| | | gctaaaaccggataggtatgagggaggcatcttcctcatattaaagcaccttcgggtgtgaacatggatggacctgcggcgcat |
| | | tagctggttggtgaggtaacggcccaccaaggcgatgatgcgtagccgacctgagagggtgaacggccacattgggactga |
| | | gacacggcccaaactcctacgggaggcagcagtagggaattttcgtcaatggggggaaccctgaacgagcaatgccgcgtg |
| | | tgtgaagaaggtcttcggatcgtaaagcactgttgtaagtgaagaatgccacatagaggaaatgctatgtgggtgacggtagct |
| | | taccagaaagccacggctaactacgtgccagcagccgcggtaatacgtaggtggcaagcgttatccggaatcattgggcgta |
| | | aagggtgcgtaggtggcacgataagtctgaagtaaaaggcaacagctcaactgttgtatgctttggaaactgtcgagctagagt |
| | | gcagaagagggcgatggaattccatgtgtagcggtaaaatgcgtagatatatggaggaacaccagtggcgaaggcggtcgc |
| | | ctggtctgtaactgacactgatgcacgaaagcgtggggagcaaataggattagataccctagtagtccacgccgtaaacgatg |
| | | agaactaagtgttggagagattcagtgctgcagttaacgcaataagttctccgcctggggagtatgcacgcaagtgtgaaactc |
| | | aaaggaattgacgggggcccgcacaagcggtggagtatgtggtttaattcgaagcaacgcgaagaaccttaccaggccttga |
| | | catggatataaatgttctagagatagaaagatagctatatatcacacaggtggtgcatggttgtcgtcagctcgtgtcgtgagatg |
| | | ttgggttaagtcccgcaacgagcgcaacccttgtcttctgttaccagcattaggttggggactcaggagagactgccggtgaca |
| | | aaccggaggaaggtggggatgacgtcaaatcatcatgccccttatggcctgggctacacacgtactacaatggcgcctacaaa |
| | | gagcagcgacaccgcgaggtggagcgaatctcataaagggcgtctcagttcggattgaagtctgcaactcgacttcatgaagt |
| | | cggaatcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagtcgtaa |
| | | caaggtatccctacgggaacgtggggatggatcacctccttt |
| Eubacterium_ | SEQ ID | atggagagtttgatcctggctcaggatgaacgctggcggcatgcctaatacatgcaagtcgaacgaagtctttaggaagcttgc |
| sp_3_1_31 | NO: 207 | ttccaaagagacttagtggcgaacgggtgagtaacacgtaggtaacctgcccatgtgcccgggataactgctggaaacggta |
| | | gctaaaaccggataggtatgagggaggcatcttcctcatattaaagcaccttcgggtgtgaacatggatggacctgcggcgcat |
| | | tagctggttggtgaggtaacggcccaccaaggcgatgatgcgtagccgacctgagagggtgaacggccacattgggactga |
| | | gacacggcccaaactcctacgggaggcagcagtagggaattttcgtcaatggggggaaccctgaacgagcaatgccgcgtg |
| | | tgtgaagaaggtcttcggatcgtaaagcactgttgtaagtgaagaatgccatatagaggaaatgctatgtgggtgacggtagctt |
| | | accagaaagccacggctaactacgtgccagcagccgcggtaatacgtaggtggcaagcgttatccggaatcattgggcgtaa |
| | | agggtgcgtaggtggcacgataagtctgaagtaaaaggcaacagctcaactgttgtatgctttggaaactgtcgagctagagtg |
| | | cagaagagggcgatggaattccatgtgtagcggtaaaatgcgtagatatatggaggaacaccagtggcgaaggcggtcgcct |
| | | ggtctgtaactgacactgatgcacgaaagcgtggggagcaaataggattagataccctagtagtccacgccgtaaacgatgag |
| | | aactaagtgttggagagattcagtgctgcagttaacgcaataagttctccgcctggggagtatgcacgcaagtgtgaaactcaa |
| | | aggaattgacgggggcccgcacaagcggtggagtatgtggtttaattcgaagcaacgcgaagaaccttaccaggccttgaca |
| | | tggatataaatgttctagagatagaaagatagctatatatcacacaggtggtgcatggttgtcgtcagctcgtgtcgtgagatgttg |
| | | ggttaagtcccgcaacgagcgcaacccttgtcttctgttaccagcattaggttggggactcaggagagactgccggtgacaaa |
| | | ccggaggaaggtggggatgacgtcaaatcatcatgccccttatggcctgggctacacacgtactacaatggcgcctacaaag |
| | | agcagcgacaccgcgaggtgaagcgaatctcataaagggcgtctcagttcggattgaagtctgcaactcgacttcatgaagtc |
| | | ggaatcgctagtaatcgcagatcagcatgctgcggtgaatacgttctcgggccttgtacacaccgcccgtcaaaccatgggag |
| | | ttggtaatacccgaagccggtggcataaccgcaaggagtgagccgtcgaaggtaggaccgatgactggggttaagtcgtaac |
| | | aaggtatccctacgggaacgtggggatggatcacctccttt |
| Eubacterium_ | SEQ ID | atggagagtttgatcctggctcaggatgaacgctggcggcatgcctaatacatgcaagtcgaacgaagtctttaggaagcttgc |
| sp_3_1_31 | NO: 208 | ttccaaagagacttagtggcgaacgggtgagtaacacgtaggtaacctgcccatgtgcccgggataactgctggaaacggta |
| | | gctaaaaccggataggtatgagggaggcatcttcctcatattaaagcaccttcgggtgtgaacatggatggacctgcggcgcat |
| | | tagctggttggtgaggtaacggcccaccaaggcgatgatgcgtagccgacctgagagggtgaacggccacattgggactga |
| | | gacacggcccaaactcctacgggaggcagcagtagggaattttcgtcaatggggggaaccctgaacgagcaatgccgcgtg |
| | | tgtgaagaaggtcttcggatcgtaaagcactgttgtaagtgaagaatgccacatagaggaaatgctatgtgggtgacggtagct |
| | | taccagaaagccacggctaactacgtgccagcagccgcggtaatacgtaggtggcaagcgttatccggaatcattgggcgta |
| | | aagggtgcgtaggtggcacgataagtctgaagtaaaaggcaacagctcaactgttgtatgctttggaaactgtcgagctagagt |
| | | gcagaagagggcgatggaattccatgtgtagcggtaaaatgcgtagatatatggaggaacaccagtggcgaaggcggtcgc |
| | | ctggtctgtaactgacactgatgcacgaaagcgtggggagcaaataggattagataccctagtagtccacgccgtaaacgatg |
| | | agaactaagtgttggagagattcagtgctgcagttaacgcaataagttctccgcctggggagtatgcacgcaagtgtgaaactc |
| | | aaaggaattgacgggggcccgcacaagcggtggagtatgtggtttaattcgaagcaacgcgaagaaccttaccaggccttga |
| | | catggatataaatgttctagagatagaaagatagctatatatcacacaggtggtgcatggttgtcgtcagctcgtgtcgtgagatg |
| | | ttgggttaagtcccgcaacgagcgcaacccttgtcttctgttaccagcattaagttggggactcaggagagactgccggtgaca |
| | | aaccggaggaaggtggggatgacgtcaaatcatcatgccccttatggcctgggctacacacgtactacaatggcgcctacaaa |
| | | gagcagcgacaccgcgaggtggagcgaatctcataaagggcgtctcagttcggattgaagtctgcaactcgacttcatgaagt |
| | | cggaatcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagtcgtaa |
| | | caaggtatccctacgggaacgtggggatggatcacctccttt |
| Eubacterium_ | SEQ ID | atggagagtttgatcctggctcaggatgaacgctggcggcatgcctaatacatgcaagtcgaacgaagtctttaggaagcttgc |
| sp_3_1_31 | NO: 209 | ttccaaagagacttagtggcgaacgggtgagtaacacgtaggtaacctgcccatgtgcccgggataactgctggaaacggta |
| | | gctaaaaccggataggtatgagggaggcatcttcctcatattaaagcaccttcgggtgtgaacatggatggacctgcggcgcat |
| | | tagctggttggtgaggtaacggcccaccaaggcgatgatgcgtagccgacctgagagggtgaacggccacattgggactga |
| | | gacacggcccaaactcctacgggaggcagcagtagggaattttcgtcaatggggggaaccctgaacgagcaatgccgcgtg |
| | | tgtgaagaaggtcttcggatcgtaaagcactgttgtaagtgaagaatgccatatagaggaaatgctatgtgggtgacggtagctt |
| | | accagaaagccacggctaactacgtgccagcagccgcggtaatacgtaggtggcaagcgttatccggaatcattgggcgtaa |
| | | agggtgcgtaggtggcacgataagtctgaagtaaaaggcaacagctcaactgttgtatgctttggaaactgtcgagctagagtg |
| | | cagaagagggcgatggaattccatgtgtagcggtaaaatgcgtagatatatggaggaacaccagtggcgaaggcggtcgcct |
| | | ggtctgtaactgacactgatgcacgaaagcgtggggagcaaataggattagataccctagtagtccacgccgtaaacgatgag |
| | | aactaagtgttggagagattcagtgctgcagttaacgcaataagttctccgcctggggagtatgcacgcaagtgtgaaactcaa |
| | | aggaattgacgggggcccgcacaagcggtggagtatgtggtttaattcgaagcaacgcgaagaaccttaccaggccttgaca |
| | | tggatataaatgttctagagatagaaagatagctatatatcacacaggtggtgcatggttgtcgtcagctcgtgtcgtgagatgttg |
| | | ggttaagtcccgcaacgagcgcaacccttgtcttctgttaccagcattaggttggggactcaggagagactgccggtgacaaa |
| | | ccggaggaaggtggggatgacgtcaaatcatcatgccccttatggcctgggctacacacgtactacaatggcgcctacaaag |
| | | agcagcgacaccgcgaggtggagcgaatctcataaagggcgtctcagttcggattgaagtctgcaactcgacttcatgaagtc |
| | | ggaatcgctagtaatcgcagatcagcatgctgcggtgaatacgttctcgggccttgtacacaccgcccgtcaaaccatgggag |
| | | ttggtaatacccgaagccggtggcataaccgcaaggagtgagccgtcgaaggtaggaccgatgactggggttaagtcgtaac |
| | | aaggtatccctacgggaacgtggggatggatcacctccttt |
| Streptococcus_ | SEQ ID | aatgagagtttgatcctggctcaggacgaacgctggcggcgtgcctaatacatgcaagtagaacgctgaagagaggagcttg |
| salivarius | NO: 210 | ctcttcttggatgagttgcgaacgggtgagtaacgcgtaggtaacctgccttgtagcgggggataactattggaaacgatagct |
| | | aataccgcataacaatgggtgacacatgtcatttatttgaaaggggcaattgctccactacaagatggacctgcgttgtattagct |
| | | agtaggtgaggtaacggctcacctaggcgacgatacatagccgacctgagagggtgatcggccacactgggactgagacac |
| | | ggcccagactcctacgggaggcagcagtagggaatcttcggcaatgggggcaaccctgaccgagcaacgccgcgtgagtg |
| | | aagaaggttttcggatcgtaaagctctgttgtaagtcaagaacgagtgtgagagtggaaagttcacactgtgacggtagcttacc |
| | | agaaagggacggctaactacgtgccagcagccgcggtaatacgtaggtcccgagcgttgtccggatttattgggcgtaaagc |
| | | gagcgcaggcggtttgataagtctgaagttaaaggctgtggctcaaccatagttcgctttggaaactgtcaaacttgagtgcaga |
| | | aggggagagtggaattccatgtgtagcggtgaaatgcgtagatatatggaggaacaccggtggcgaaagcggctctctggtc |
| | | tgtaactgacgctgaggctcgaaagcgtggggagcgaacaggattagataccctggtagtccacgccgtaaacgatgagtgc |
| | | taggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| salivarius | NO: 211 | ctcttcttggatgagttgcgaacgggtgagtaacgcgtaggtaacctgccttgtagcgggggataactattggaaacgatagct |
| | | aataccgcataacaatgaatgactcatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| salivarius | NO: 212 | ctcttcttggatgagttgcgaacgggtgagtaacgcgtaggtaacctgccttgtagcgggggataactattggaaacgatagct |
| | | aataccgcataacaatgaatgactcatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgaccttcctggctcaggacgaacgctggcggcgtgcctaatacatgcaagtagaacgctgaagagaggagc |
| salivarius | NO: 213 | ttgctcttcttggatgagttgcgaacgggtgagtaacgcgtaggtaacctgccttgtagcgggggataactattggaaacgatag |
| | | ctaataccgcataacaatgaatgactcatgtcatttatttgaaaggggcaattgctccactacaagatggacctgcgttgtattagc |
| | | tagtaggtgaggtaacggctcacctaggcgacgatacatagccgacctgagagggtgatcggccacactgggactgagaca |
| | | cggcccagactcctacgggaggcagcagtagggaatcttcggcaatgggggcaaccctgaccgagcaacgccgcgtgagt |
| | | gaagaaggttttcggatcgtaaagctctgttgtaagtcaagaacgagtgtgagagtggaaagttcacactgtgacggtagcttac |
| | | cagaaagggacggctaactacgtgccagcagccgcggtaatacgtaggtcccgagcgttgtccggatttattgggcgtaaag |
| | | cgagcgcaggcggtttgataagtctgaagttaaaggctgtggctcaaccatagttcgctttggaaactgtcaaacttgagtgcag |
| | | aaggggagagtggaattccatgtgtagcggtgaaatgcgtagatatatggaggaacaccggtggcgaaagcggctctctggt |
| | | ctgtaactgacgctgaggctcgaaagcgtggggagcgaacaggattagataccctggtagtccacgccgtaaacgatgagtg |
| | | ctaggtgaggatcctttccgggattcagtgccgcagctaacgcattaagcactccgcctggggagtacgaccgcaaggttgaa |
| | | actcaaaggaattgacgggggcccgcacaagcggtggagcatgtggtttaattcgaagcaacgcgaagaaccttaccaggtc |
| | | ttgacatcccgatgctatttctagagatagaaagttacttcggtacatcggtgacaggtggtgcatggttgtcgtcagctcgtgtcg |
| | | tgagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| salivarius | NO: 214 | ctcttcttggatgagttgcgaacgggtgagtaacgcgtaggtaacctgccttgtagcgggggataactattggaaacgatagct |
| | | aataccgcataacaatgaatgactcatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| salivarius | NO: 215 | ctcttcttggatgagttgcgaacgggtgagtaacgcgtaggtaacctgccttgtagcgggggataactattggaaacgatagct |
| | | aataccgcataacaatgaatgactcatgtcatttatttgaaaggggcaattgctccactacaagatggacctgcgttgtattagcta |
| | | gtaggtgaggtaacggctcacctaggcgacgatacatagccgacctgagagggtgatcggccacactgggactgagacacg |
| | | gcccagactcctacgggaggcagcagtagggaatcttcggcaatgggggcaaccctgaccgagcaacgccgcgtgagtga |
| | | agaaggttttcggatcgtaaagctctgttgtaagtcaagaacgagtgtgagagtggaaagttcacactgtgacggtagcttacca |
| | | gaaagggacggctaactacgtgccagcagccgcggtaatacgtaggtcccgagcgttgtccggatttattgggcgtaaagcg |
| | | agcgcaggcggtttgataagtctgaagttaaaggctgtggctcaaccatagttcgctttggaaactgtcaaacttgagtgcagaa |
| | | ggggagagtggaattccatgtgtagcggtgaaatgcgtagatatatggaggaacaccggtggcgaaagcggctctctggtct |
| | | gtaactgacgctgaggctcgaaagcgtggggagcgaacaggattagataccctggtagtccacgccgtaaacgatgagtgct |
| | | aggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagaggagcttg |
| salivarius | NO: 216 | ctcttcttggatgagttgcgaacgggtgagtaacgcgtaggtaacctgccttgtagcgggggataactattggaaacgatagct |
| | | aataccgcataacaatgggtgacacatgtcatttatttgaaaggggcaattgctccactacaagatggacctgcgttgtattagct |
| | | agtaggtgaggtaacggctcacctaggcgacgatacatagccgacctgagagggtgatcggccacactgggactgagacac |
| | | ggcccagactcctacgggaggcagcagtagggaatcttcggcaatgggggcaaccctgaccgagcaacgccgcgtgagtg |
| | | aagaaggttttcggatcgtaaagactgttgtaagtcaagaacgagtgtgagagtggaaagttcacactgtgacggtagcttacc |
| | | agaaagggacggctaactacgtgccagcagccgcggtaatacgtaggtcccgagcgttgtccggatttattgggcgtaaagc |
| | | gagcgcaggcggtttgataagtctgaagttaaaggctgtggctcaaccatagttcgctttggaaactgtcaaacttgagtgcaga |
| | | aggggagagtggaattccatgtgtagcggtgaaatgcgtagatatatggaggaacaccggtggcgaaagcggctctctggtc |
| | | tgtaactgacgctgaggctcgaaagcgtggggagcgaacaggattagataccctggtagtccacgccgtaaacgatgagtgc |
| | | taggtgttggatcctttccgggattcagtgccgcagctaacgcattaagcactccgcctggggagtacgaccgcaaggttgaaa |
| | | ctcaaaggaattgacgggggcccgcacaagcggtggagcatgtggtttaattcgaagcaacgcgaagaaccttaccaggtct |
| | | tgacatcccgatgctatttctagagatagaaagttacttcggtacatcggtgacaggtggtgcatggttgtcgtcagctcgtgtcgt |
| | | gagatgttgggttaagtcccgcaacgagcgcaacccctattgttagttgccatcattcagttgggcactctagcgagactgccg |
| | | gtaataaaccggaggaaggtggggatgacgtcaaatcatcatgccccttatgacctgggctacacacgtgctacaatggttggt |
| | | acaacgagttgcgagtcggtgacggcaagctaatctcttaaagccaatctcagttcggattgtaggctgcaactcgcctacatg |
| | | aagtcggaatcgctagtaatcgcggatcagcacgccgcggtgaatacgttcccgggccttgtacacaccgcccgtcacacca |
| | | cgagagtttgtaacacccgaagtcggtgaggtaaccttttggagccagccgcctaaggtgggatagatgattggggtgaagtc |
| | | gtaacaaggtagccgtatcggaaggtgcggctggatcacctcctt |
| Parabacteroides_ | SEQ ID | cgaagagtttgatcctggctcaggatgaacgctagcgacaggcttaacacatgcaagtcgaggggcagcacaggtagcaata |
| distasonis | NO: 217 | ccgggtggcgaccggcgcacgggtgagtaacgcgtatgcaacttacctatcagagggggataacccggcgaaagtcggac |
| | | taataccgcatgaagcaggggtcccgcatggggatatttgctaaagattcatcgctgatagataggcatgcgttccattaggca |
| | | gttggcggggtaacggcccaccaaaccgacgatggataggggttctgagaggaaggtcccccacattggtactgagacacg |
| | | gaccaaactcctacgggaggcagcagtgaggaatattggtcaatgggcgtaagcctgaaccagccaagtcgcgtgagggat |
| | | gaaggttctatggatcgtaaacctcttttataagggaataaagtgcgggacgtgtcctgttttgtatgtaccttatgaataaggatcg |
| | | gctaactccgtgccagcagccgcggtaatacggaggatccgagcgttatccggatttattgggtttaaagggtgcgtaggcgg |
| | | ccttttaagtcagcggtgaaagtctgtggctcaaccatagaattgccgttgaaactggggggcttgagtatgtttgaggcaggcg |
| | | gaatgcgtggtgtagcggtgaaatgcttagatatcacgcagaaccccgattgcgaaggcagcctgccaagccatgactgacg |
| | | ctgatgcacgaaagcgtggggatcaaacaggattagataccctggtagtccacgcagtaaacgatgatcactagctgtttgcg |
| | | atacagtgtaagcggcacagcgaaagcgttaagtgatccacctggggagtacgccggcaacggtgaaactcaaaggaattg |
| | | acgggggcccgcacaagcggaggaacatgtggtttaattcgatgatacgcgaggaaccttacccgggtttgaacgcattcgg |
| | | accgaggtggaaacaccttttctagcaatagccgtttgcgaggtgctgcatggttgtcgtcagctcgtgccgtgaggtgtcggct |
| | | taagtgccataacgagcgcaacccttgccactagttactaacaggtaaagctgaggactctggtgggactgccagcgtaagct |
| | | gcgaggaaggcggggatgacgtcaaatcagcacggcccttacatccggggcgacacacgtgttacaatggcgtggacaaa |
| | | gggaggccacctggcgacagggagcgaatccccaaaccacgtctcagttcggatcggagtctgcaacccgactccgtgaag |
| | | ctggattcgctagtaatcgcgcatcagccatggcgcggtgaatacgttcccgggccttgtacacaccgcccgtcaagccatgg |
| | | gagccgggggtacctgaagtccgtaaccgcgaggatcggcctagggtaaaactggtgactggggctaagtcgtaacaaggt |
| | | agccgtaccggaaggtgcggctggaacacctccttt |
| Parabacteroides_ | SEQ ID | cgaagagtttgatcctggctcaggatgaacgctagcgacaggcttaacacatgcaagtcgaggggcagcacaggtagcaata |
| distasonis | NO: 218 | ccgggtggcgaccggcgcacgggtgagtaacgcgtatgcaacttgcctatcagagggggataacccggcgaaagtcggac |
| | | taataccgcatgaagcaggggccccgcatggggatatttgctaaagattcatcgctgatagataggcatgcgttccattaggca |
| | | gttggcggggtaacggcccaccaaaccgacgatggataggggttctgagaggaaggtcccccacattggtactgagacacg |
| | | gaccaaactcctacgggaggcagcagtgaggaatattggtcaatgggcgcgagcctgaaccagccaagtcgcgtgagggat |
| | | gaaggttctatggatcgtaaacctcttttataagggaataaagtgcgggacgtgtcctgttttgtatgtaccttatgaataaggatcg |
| | | gctaactccgtgccagcagccgcggtaatacggaggatccgagcgttatccggatttattgggtttaaagggtgcgtaggcgg |
| | | ccttttaagtcagcggtgaaagtctgtggctcaaccatagaattgccgttgaaactggggggcttgagtatgtttgaggcaggcg |
| | | gaatgcgtggtgtagcggtgaaatgcttagatatcacgcagaaccccgattgcgaaggcagcctgccaagccatgactgacg |
| | | ctgatgcacgaaagcgtggggatcaaacaggattagataccctggtagtccacgcagtaaacgatgatcactagctgtttgcg |
| | | atacagtgtaagcggcacagcgaaagcgttaagtgatccacctggggagtacgccggcaacggtgaaactcaaaggaattg |
| | | acgggggcccgcacaagcggaggaacatgtggtttaattcgatgatacgcgaggaaccttacccgggtttgaacgcattcgg |
| | | accgaggtggaaacaccttttctagcaatagccgtttgcgaggtgctgcatggttgtcgtcagctcgtgccgtgaggtgtcggct |
| | | taagtgccataacgagcgcaacccttgccactagttactaacaggtaaagctgaggactctggtgggactgccagcgtaagct |
| | | gcgaggaaggcggggatgacgtcaaatcagcacggcccttacatccggggcgacacacgtgttacaatggcgtggacaaa |
| | | gggatgccacctggcgacagggagcgaatccccaaaccacgtctcagttcggatcggagtctgcaacccgactccgtgaag |
| | | ctggattcgctagtaatcgcgcatcagccatggcgcggtgaatacgttcccgggccttgtacacaccgcccgtcaagccatgg |
| | | gagccgggggtacctgaagtccgtaaccgaaaggatcggcctagggtaaaactggtgactggggctaagtcgtaacaaggt |
| | | agccgtaccggaaggtgcggctggaacacctccttt |
| Parabacteroides_ | SEQ ID | cgaagagtttgatcctggctcaggatgaacgctagcgacaggcttaacacatgcaagtcgaggggcagcacaggtagcaata |
| distasonis | NO: 219 | ccgggtggcgaccggcgcacgggtgagtaacgcgtatgcaacttacctatcagagggggataacccggcgaaagtcggac |
| | | taataccgcatgaagcaggggtcccgcatggggatatttgctaaagattcatcgctgatagataggcatgcgttccattaggca |
| | | gttggcggggtaacggcccaccaaaccgacgatggataggggttctgagaggaaggtcccccacattggtactgagacacg |
| | | gaccaaactcctacgggaggcagcagtgaggaatattggtcaatgggcgcgagcctgaaccagccaagtcgcgtgagggat |
| | | gaaggttctatggatcgtaaacctcttttataagggaataaagtgcgggacgtgtcctgttttgtatgtaccttatgaataaggatcg |
| | | gctaactccgtgccagcagccgcggtaatacggaggatccgagcgttatccggatttattgggtttaaagggtgcgtaggcgg |
| | | ccttttaagtcagcggtgaaagtctgtggctcaaccatagaattgccgttgaaactggggggcttgagtatgtttgaggcaggcg |
| | | gaatgcgtggtgtagcggtgaaatgcttagatatcacgcagaaccccgattgcgaaggcagcctgccaagccatgactgacg |
| | | ctgatgcacgaaagcgtggggatcaaacaggattagataccctggtagtccacgcagtaaacgatgatcactagctgtttgcg |
| | | atacagtgtaagcggcacagcgaaagcgttaagtgatccacctggggagtacgccggcaacggtgaaactcaaaggaattg |
| | | acgggggcccgcacaagcggaggaacatgtggtttaattcgatgatacgcgaggaaccttacccgggtttgaacgcattcgg |
| | | accgaggtggaaacaccttttctagcaatagccgtttgcgaggtgctgcatggttgtcgtcagctcgtgccgtgaggtgtcggct |
| | | taagtgccataacgagcgcaacccttgccactagttactaacaggtgatgctgaggactctggtgggactgccagcgtaagct |
| | | gcgaggaaggcggggatgacgtcaaatcagcacggcccttacatccggggcgacacacgtgttacaatggcgtggacaaa |
| | | gggaagccacctggcgacagggagcgaatccccaaaccacgtctcagttcggatcggagtctgcaacccgactccgtgaag |
| | | ctggattcgctagtaatcgcgcatcagccatggcgcggtgaatacgttcccgggccttgtacacaccgcccgtcaagccatgg |
| | | gagccgggggtacctgaagtccgtaaccgaaaggatcggcctagggtaaaactggtgactggggctaagtcgtaacaaggt |
| | | agccgtaccggaaggtgcggctggaacacctccttt |
| Parabacteroides_ | SEQ ID | cgaagagtttgatcctggctcaggatgaacgctagcgacaggcttaacacatgcaagtcgaggggcagcgggggtagcaat |
| distasonis | NO: 220 | acccgccggcgaccggcgcacgggtgagtaacgcgtatgcaacttacctatcagagggggataacccggcgaaagtcgga |
| | | ctaataccgcatgaagcaggggccccgcatggggatatttgctaaagattcatcgctgatagataggcatgcgttccattaggc |
| | | agttggcggggtaacggcccaccaaaccgacgatggataggggttctgagaggaaggtcccccacattggtactgagacac |
| | | ggaccaaactcctacgggaggcagcagtgaggaatattggtcaatgggcgcgagcctgaaccagccaagtcgcgtgaggg |
| | | atgaaggttctatggatcgtaaacctcttttataagggaataaagtgcgggacgtgtcctgttttgtatgtaccttatgaataaggat |
| | | cggctaactccgtgccagcagccgcggtaatacggaggatccgagcgttatccggatttattgggtttaaagggtgcgtaggc |
| | | ggccttttaagtcagcggtgaaagtctgtggctcaaccatagaattgccgttgaaactggggggcttgagtatgtttgaggcagg |
| | | cggaatgcgtggtgtagcggtgaaatgcttagatatcacgcagaaccccgattgcgaaggcagcctgccaagccatgactga |
| | | cgctgatgcacgaaagcgtggggatcaaacaggattagataccctggtagtccacgcagtaaacgatgatcactagctgtttg |
| | | cgatacagtgtaagcggcacagcgaaagcgttaagtgatccacctggggagtacgccggcaacggtgaaactcaaaggaat |
| | | tgacgggggcccgcacaagcggaggaacatgtggtttaattcgatgatacgcgaggaaccttacccgggtttgaacgcattcg |
| | | gaccgaggtggaaacaccttttctagcaatagccgtttgcgaggtgctgcatggttgtcgtcagctcgtgccgtgaggtgtcgg |
| | | cttaagtgccataacgagcgcaacccttgccactagttactaacaggtaatgctgaggactctggtgggactgccagcgtaagc |
| | | tgcgaggaaggcggggatgacgtcaaatcagcacggcccttacatccggggcgacacacgtgttacaatggcgtggacaaa |
| | | gggaggccacctggcgacagggagcgaatccccaaaccacgtctcagttcggatcggagtctgcaacccgactccgtgaag |
| | | ctggattcgctagtaatcgcgcatcagccatggcgcggtgaatacgttcccgggccttgtacacaccgcccgtcaagccatgg |
| | | gagccgggggtacctgaagtccgtaaccgcgaggatcggcctagggtaaaactggtgactggggctaagtcgtaacaaggt |
| | | agccgtaccggaaggtgcggctggaacacctccttt |
| Parabacteroides_ | SEQ ID | cgaagagtttgatcctggctcaggatgaacgctagcgacaggcttaacacatgcaagtcgaggggcagcacaggtagcaata |
| distasonis | NO: 221 | ccgggtggcgaccggcgcacgggtgagtaacgcgtatgcaacttacctatcagagggggataacccggcgaaagtcggac |
| | | taataccgcatgaagcaggggtcccgcatggggatatttgctaaagattcatcgctgatagataggcatgcgttccattaggca |
| | | gttggcggggtaacggcccaccaaaccgacgatggataggggttctgagaggaaggtcccccacattggtactgagacacg |
| | | gaccaaactcctacgggaggcagcagtgaggaatattggtcaatgggcgtaagcctgaaccagccaagtcgcgtgagggat |
| | | gaaggttctatggatcgtaaacctcttttataagggaataaagtgcgggacgtgtcctgttttgtatgtaccttatgaataaggatcg |
| | | gctaactccgtgccagcagccgcggtaatacggaggatccgagcgttatccggatttattgggtttaaagggtgcgtaggcgg |
| | | ccttttaagtcagcggtgaaagtctgtggctcaaccatagaattgccgttgaaactggggggcttgagtatgtttgaggcaggcg |
| | | gaatgcgtggtgtagcggtgaaatgcttagatatcacgcagaaccccgattgcgaaggcagcctgccaagccatgactgacg |
| | | ctgatgcacgaaagcgtggggatcaaacaggattagataccctggtagtccacgcagtaaacgatgatcactagctgtttgcg |
| | | atacagtgtaagcggcacagcgaaagcgttaagtgatccacctggggagtacgccggcaacggtgaaactcaaaggaattg |
| | | acgggggcccgcacaagcggaggaacatgtggtttaattcgatgatacgcgaggaaccttacccgggtttgaacgcattcgg |
| | | accgaggtggaaacaccttttctagcaatagccgtttgcgaggtgctgcatggttgtcgtcagctcgtgccgtgaggtgtcggct |
| | | taagtgccataacgagcgcaacccttgccactagttactaacaggtaaagctgaggactctggtgggactgccagcgtaagct |
| | | gcgaggaaggcggggatgacgtcaaatcagcacggcccttacatccggggcgacacacgtgttacaatggcgtggacaaa |
| | | gggaagccacctggcgacagggagcgaatccccaaaccacgtctcagttcggatcggagtctgcaacccgactccgtgaag |
| | | ctggattcgctagtaatcgcgcatcagccatggcgcggtgaatacgttcccgggccttgtacacaccgcccgtcaagccatgg |
| | | gagccgggggtacctgaagtccgtaaccgcgaggatcggcctagggtaaaactggtgactggggctaagtcgtaacaaggt |
| | | agccgtaccggaaggtgcggctggaacacctccttt |
| Clostridiaceae_ | SEQ ID | atggagagtttgatcctggctcaggatgaacgctggcggcatgcctaatacatgcaagtcgaacgaagtgaagatagcttgcta |
| bacterium_ | NO: 222 | ttggaacttagtggcgaacgggtgagtaacacgtagataacctgcctgtatgaccgggataacagttggaaacgactgctaata |
| JC118 | | ccggataggcagagaggaggcatctcttctctgttaaagttgggacacaacgcaaacagatggatctgcggtgcattagctagt |
| | | tggtgaggtaacggcccaccaaggcgatgatgcatagccggcctgagagggcgaacggccacattgggactgagacacgg |
| | | cccaaactcctacgggaggcagcagtagggaattttcggcaatgggggaaaccctgaccgagcaatgccgcgtgagtgaag |
| | | acggccttcgggttgtaaagctctgttgtaagggaagaacggcatagagagggaatgctctatgagtgacggtaccttaccag |
| | | aaagccacggctaactacgtgccagcagccgcggtaatacgtaggtggcaagcgttatccggaattattgggcgtaaagggt |
| | | gcgtaggcggcgagataagtctgaggtaaaagcccgtggctcaaccacggtaagccttggaaactgtctggctggagtgcag |
| | | gagaggacaatggaattccatgtgtagcggtaaaatgcgtagatatatggaggaacaccagtggcgaaggcggttgtctggc |
| | | ctgtaactgacgctgaagcacgaaagcgtggggagcaaataggattagataccctagtagtccacgccgtaaacgatgagaa |
| | | ctaagtgttggggaaactcagtgctgcagttaacgcaataagttctccgcctggggagtatgcacgcaagtgtgaaactcaaag |
| | | gaattgacgggggcccgcacaagcggtggagtatgtggtttaattcgacgcaacgcgaagaaccttaccaggccttgacatg |
| | | gtatcaaaggccctagagatagggagatagttatggtacacacaggtggtgcatggttgtcgtcagctcgtgtcgtgagatgttg |
| | | ggttaagtcccgcaacgagcgcaacccttgtttctagttaccaacagtaagatggggactctagagagactgccggtgacaaa |
| | | ccggaggaaggtggggatgacgtcaaatcatcatgccccttatggcctgggctacacacgtactacaatggcgtctacaaaga |
| | | gcagcgagcaggtgactgtgagcgaatctcataaaggacgtctcagttcggattgaagtctgcaactcgacttcatgaagtcgg |
| | | aatcgctagtaatcgcggatcagcatgccgcggtgaatacgttctcgggccttgtacacaccgcccgtcaaaccatgggagtt |
| | | gataatacccgaagccggtggcctaaccatttatggagggagccgtcgaaggtaggatcgatgactggggttaagtcgtaaca |
| | | aggtatccctacgggaacgtggggatggatcacctccttt |
| Anaerostipes_ | SEQ ID | acctaccctgtacagggggataacagtcagaaatgactgctaataccgcataagaccacagcaccgcatggtgcaggggtaa |
| hadrus | NO: 223 | aaactccggtggtacaggatggacccgcgtctgattagctggttggtgaggtaacggctcaccaaggcgacgatcagtagcc |
| | | ggcttgagagagtgaacggccacattgggactgagacacggcccaaactcctacgggaggcagcagtggggaatattgcac |
| | | aatgggggaaaccctgatgcagcgacgccgcgtgagtgaagaagtatctcggtatgtaaagctctatcagcagggaagaaaa |
| | | tgacggtacctgactaagaagccccggctaactacgtgccagcagccgcggtaatacgtagggggcaagcgttatccggaat |
| | | tactgggtgtaaagggtgcgtaggtggtatggcaagtcagaagtgaaaacccagggcttaactctgggactgcttttgaaactg |
| | | tcagactggagtgcaggagaggtaagcggaattcctagtgtagcggtgaaatgcgtagatattaggaggaacatcagtggcg |
| | | aaggcggcttactggactgaaactgacactgaggcacgaaagcgtggggagcaaacaggattagataccctggtagtccac |
| | | gccgtaaacgatgaatactaggtgtcggggccgtagaggcttcggtgccgcagccaacgcagtaagtattccacctggggag |
| | | tacgttcgcaagaatgaaactcaaaggaattgacggggacccgcacaagcggtggagcatgtggtttaattcgaagcaacgc |
| | | gaagaaccttacctggtcttgacatccttctgaccggtccttaaccggacctttccttcgggacaggagagacaggtggtgcatg |
| | | gttgtcgtcagctcgtgtcgtgagatgttgggttaagtcccgcaacgagcgcaacccctatctttagtagccagca |
| Clostridium_ | SEQ ID | atggagagtttgatcctggctcaggatgaacgctggcggcatgcctaatacatgcaagtcgaacgaagactctaggaagcttg |
| innocuum | NO: 224 | cttccaaagagacttagtggcgaacgggtgagtaacacgtaggtaacctgcccatgtgtccgggataactgctggaaacggta |
| | | gctaaaaccggataggtatacagagcgcatgctcagtatattaaagcgcccatcaaggcgtgaacatggatggacctgcggc |
| | | gcattagctagttggtgaggtaacggcccaccaaggcgatgatgcgtagccggcctgagagggtaaacggccacattggga |
| | | ctgagacacggcccaaactcctacgggaggcagcagtagggaattttcgtcaatgggggaaaccctgaacgagcaatgccg |
| | | cgtgagtgaagaaggtcttcggatcgtaaagctctgttgtaagtgaagaacggctcatagaggaaatgctatgggagtgacgg |
| | | tagcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggaaacaaataccctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcga |
| | | ccacaaagagcagcgacttggtgacaagaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Clostridium_ | SEQ ID | atggagagtttgatcctggctcaggatgaacgctggcggcatgcctaatacatgcaagtcgaacgaagtcttcaggaagcttgc |
| innocuum | NO: 225 | ttccaaaaagacttagtggcgaacgggtgagtaacacgtaggtaacctgcccatgtgtccgggataactgctggaaacggtag |
| | | ctaaaaccggataggtatacggagcgcatgctctgtatattaaagcgcccatcaaggcgtgaacatggatggacctgcggcgc |
| | | attagctagttggtgaggtaacggcccaccaaggcgatgatgcgtagccggcctgagagggtaaacggccacattgggactg |
| | | agacacggcccaaactcctacgggaggcagcagtagggaattttcgtcaatgggggaaaccctgaacgagcaatgccgcgt |
| | | gagtgaagaaggtcttcggatcgtaaagactgttgtaagtgaagaacggctcatagaggaaatgctatgggagtgacggtag |
| | | cttaccagaaagccacggctaactacgtgccagcagccgcggtaatacgtaggtggcaagcgttatccggaatcattgggcgt |
| | | aaagggtgcgtaggtggcgtactaagtctgtagtaaaaggcaatggctcaaccattgtaagctatggaaactggtatgctggag |
| | | tgcagaagagggcgatggaattccatgtgtagcggtaaaatgcgtagatatatggaggaacaccagtggcgaaggcggtcg |
| | | cctggtctgtaactgacactgaggcacgaaagcgtggggagcaaataggattagataccctagtagtccacgccgtaaacgat |
| | | gagaactaagtgttggaggaattcagtgctgcagttaacgcaataagttctccgcctggggagtatgcacgcaagtgtgaaact |
| | | caaaggaattgacgggggcccgcacaagcggtggagtatgtggtttaattcgaagcaacgcgaagaaccttaccaggccttg |
| | | acatggaaacaaataccctagagatagggggataattatggatcacacaggtggtgcatggttgtcgtcagctcgtgtcgtgag |
| | | atgttgggttaagtcccgcaacgagcgcaacccttgtcgcatgttaccagcatcaagttggggactcatgcgagactgccggtg |
| | | acaaaccggaggaaggtggggatgacgtcaaatcatcatgccccttatggcctgggctacacacgtactacaatggcgacca |
| | | caaagagcagcgacttggtgacaagaagcgaatctcataaaggtcgtctcagttcggattgaagtctgcaactcgacttcatga |
| | | agtcggaatcgctagtaatcgcagatcagcatgctgcggtgaatacgttctcgggccttgtacacaccgcccgtcaaaccatgg |
| | | gagtcagtaatacccgaagccggtggcataaccgtaaggagtgagccgtcgaaggtaggaccgatgactggggttaagtcgt |
| | | aacaaggtatccctacgggaacgtggggatggatcacctccttt |
| Clostridium_ | SEQ ID | atggagagtttgatcctggctcaggatgaacgctggcggcatgcctaatacatgcaagtcgaacgaagtcttcaggaagcttgc |
| innocuum | NO: 226 | ttccaaaaagacttagtggcgaacgggtgagtaacacgtaggtaacctgcccatgtgtccgggataactgctggaaacggtag |
| | | ctaaaaccggataggtatacggagcgcatgctctgtatattaaagcgcccttcaaggcgtgaacatggatggacctgcgacgc |
| | | attagctagttggtgaggtaacggcccaccaaggcgatgatgcgtagccggcctgagagggtaaacggccacattgggactg |
| | | agacacggcccaaactcctacgggaggcagcagtagggaattttcgtcaatgggggaaaccctgaacgagcaatgccgcgt |
| | | gagtgaagaaggtcttcggatcgtaaagctctgttgtaagtgaagaacggctcatagaggaaatgctatgggagtgacggtag |
| | | cttaccagaaagccacggctaactacgtgccagcagccgcggtaatacgtaggtggcaagcgttatccggaatcattgggcgt |
| | | aaagggtgcgtaggtggcgtactaagtctgtagtaaaaggcaatggctcaaccattgtaagctatggaaactggtatgctggag |
| | | tgcagaagagggcgatggaattccatgtgtagcggtaaaatgcgtagatatatggaggaacaccagtggcgaaggcggtcg |
| | | cctggtctgtaactgacactgaggcacgaaagcgtggggagcaaataggattagataccctagtagtccacgccgtaaacgat |
| | | gagaactaagtgttggagaaattcagtgctgcagttaacgcaataagttctccgcctggggagtatgcacgcaagtgtgaaact |
| | | caaaggaattgacgggggcccgcacaagcggtggagtatgtggtttaattcgaagcaacgcgaagaaccttaccaggccttg |
| | | acatggaaacaaataccctagagatagggggataattatggatcacacaggtggtgcatggttgtcgtcagctcgtgtcgtgag |
| | | atgttgggttaagtcccgcaacgagcgcaacccttgtcgcatgttaccagcatcaagttggggactcatgcgagactgccggtg |
| | | acaaaccggaggaaggtggggatgacgtcaaatcatcatgccccttatggcctgggctacacacgtactacaatggcgacca |
| | | caaagagcagcgacttggtgacaagaagcgaatctcataaaggtcgtctcagttcggattgaagtctgcaactcgacttcatga |
| | | agtcggaatcgctagtaatcgcagatcagcatgctgcggtgaatacgttctcgggccttgtacacaccgcccgtcaaaccatgg |
| | | gagtcagtaatacccgaagccggtggcataaccgtaaggagtgagccgtcgaaggtaggaccgatgactggggttaagtcgt |
| | | aacaaggtatccctacgggaacgtggggatggatcacctccttt |
| Clostridium_ | SEQ ID | atggagagtttgatcctggctcaggatgaacgctggcggcatgcctaatacatgcaagtcgaacgaagtcttcaggaagcttgc |
| innocuum | NO: 227 | ttccaaaaagacttagtggcgaacgggtgagtaacacgtaggtaacctgcccatgtgtccgggataactgctggaaacggtag |
| | | ctaaaaccggataggtatacggagcgcatgctcagtatattaaagcgcccatcaaggcgtgaacatggatggacctgcggcg |
| | | cattagctagttggtgaggtaacggcccaccaaggcgatgatgcgtagccggcctgagagggtaaacggccacattgggact |
| | | gagacacggcccaaactcctacgggaggcagcagtagggaattttcgtcaatgggggaaaccctgaacgagcaatgccgc |
| | | gtgagtgaagaaggtcttcggatcgtaaagctctgttgtaagtgaagaacggctcatagaggaaatgctatgggagtgacggt |
| | | agcttaccagaaagccacggctaactacgtgccagcagccgcggtaatacgtaggtggcaagcgttatccggaatcattggg |
| | | cgtaaagggtgcgtaggtggcgtactaagtctgtagtaaaaggcaatggctcaaccattgtaagctatggaaactggtatgctg |
| | | gagtgcagaagagggcgatggaattccatgtgtagcggtaaaatgcgtagatatatggaggaacaccagtggcgaaggcgg |
| | | tcgcctggtctgtaactgacactgaggcacgaaagcgtggggagcaaataggattagataccctagtagtccacgccgtaaac |
| | | gatgagaactaagtgttggaggaattcagtgctgcagttaacgcaataagttctccgcctggggagtatgcacgcaagtgtgaa |
| | | actcaaaggaattgacgggggcccgcacaagcggtggagtatgtggtttaattcgaagcaacgcgaagaaccttaccaggcc |
| | | ttgacatggaaacaaataccctagagatagggggataattatggatcacacaggtggtgcatggttgtcgtcagctcgtgtcgtg |
| | | agatgttgggttaagtcccgcaacgagcgcaacccttgtcgcatgttaccagcatcaagttggggactcatgcgagactgccg |
| | | gtgacaaaccggaggaaggtggggatgacgtcaaatcatcatgccccttatggcctgggctacacacgtactacaatggcga |
| | | ccacaaagagcagcgacttggtgacaagaagcgaatctcataaaggtcgtctcagttcggattgaagtctgcaactcgacttca |
| | | tgaagtcggaatcgctagtaatcgcagatcagcatgctgcggtgaatacgttctcgggccttgtacacaccgcccgtcaaacca |
| | | tgggagtcagtaatacccgaagccggtggcataaccgtaaggagtgagccgtcgaaggtaggaccgatgactggggttaag |
| | | tcgtaacaaggtatccctacgggaacgtggggatggatcacctccttt |
| Clostridium_ | SEQ ID | atggagagtttgatcctggctcaggatgaacgctggcggcatgcctaatacatgcaagtcgaacgaagtctttaggaagcttgc |
| innocuum | NO: 228 | ttccaaaaagacttagtggcgaacgggtgagtaacacgtaggtaacctgcccatgtgtccgggataactgctggaaacggtag |
| | | ctaaaaccggataggtatacggagcgcatgctctgtatattaaagcgcccatcaaggcgtgaacatggatggacctgcggcgc |
| | | attagctagttggtgaggtaacggcccaccaaggcgatgatgcgtagccggcctgagagggtaaacggccacattgggactg |
| | | agacacggcccaaactcctacgggaggcagcagtagggaattttcgtcaatgggggaaaccctgaacgagcaatgccgcgt |
| | | gagtgaagaaggtcttcggatcgtaaagctctgttgtaagtgaagaacggctcatagaggaaatgctatgggagtgacggtag |
| | | cttaccagaaagccacggctaactacgtgccagcagccgcggtaatacgtaggtggcaagcgttatccggaatcattgggcgt |
| | | aaagggtgcgtaggtggcgtactaagtctgtagtaaaaggcaatggctcaaccattgtaagctatggaaactggtatgctggag |
| | | tgcagaagagggcgatggaattccatgtgtagcggtaaaatgcgtagatatatggaggaacaccagtggcgaaggcggtcg |
| | | cctggtctgtaactgacactgaggcacgaaagcgtggggagcaaataggattagataccctagtagtccacgccgtaaacgat |
| | | gagaactaagtgttggaggaattcagtgctgcagttaacgcaataagttctccgcctggggagtatgcacgcaagtgtgaaact |
| | | caaaggaattgacgggggcccgcacaagcggtggagtatgtggtttaattcgaagcaacgcgaagaaccttaccaggccttg |
| | | acatggaaacaaataccctagagatagggggataattatggatcacacaggtggtgcatggttgtcgtcagctcgtgtcgtgag |
| | | atgttgggttaagtcccgcaacgagcgcaacccttgtcgcatgttaccagcatcaagttggggactcatgcgagactgccggtg |
| | | acaaaccggaggaaggtggggatgacgtcaaatcatcatgccccttatggcctgggctacacacgtactacaatggcgacca |
| | | caaagagcagcgacttggtgacaagaagcgaatctcataaaggtcgtctcagttcggattgaagtctgcaactcgacttcatga |
| | | agtcggaatcgctagtaatcgcagatcagcatgctgcggtgaatacgttctcgggccttgtacacaccgcccgtcaaaccatgg |
| | | gagtcagtaatacccgaagccggtggcataaccgtaaggagtgagccgtcgaaggtaggaccgatgactggggttaagtcgt |
| | | aacaaggtatccctacgggaacgtggggatggatcacctccttt |
| Clostridium_ | SEQ ID | atgagagttcgatcctggctcaggatgaacgctggcggcgtgcttaacacatgcaagtcgagcgaagcggtttcaatgaagttt |
| hathewayi | NO: 229 | tcggatggaattgaaattgacttagcggcggacgggtgagtaacgcgtgggtaacctgccttacactgggggataacagttag |
| | | aaatgactgctaataccgcataagcgcacagggccgcatggtctggtgtgaaaaactccggtggtgtaagatggacccgcgt |
| | | ctgattaggtagttggtggggtaacggcccaccaagccgacgatcagtagccgacctgagagggtgaccggccacattggg |
| | | actgagacacggcccaaactcctacgggaggcagcagtggggaatattggacaatgggcgaaagcctgatccagcgacgc |
| | | cgcgtgagtgaagaagtatttcggtatgtaaagctctatcagcagggaagaaaatgacggtacctgactaagaagccccggct |
| | | aactacgtgccagcagccgcggtaatacgtagggggcaagcgttatccggatttactgggtgtaaagggagcgtagacggtt |
| | | aagcaagtctgaagtgaaagcccggggctcaaccccggtactgctttggaaactgtttgacttgagtgcaggagaggtaagtg |
| | | gaattcctagtgtagcggtgaaatgcgtagatattaggaggaacaccagtggcgaaggcggcttactggactgtaactgacgtt |
| | | gaggctcgaaagcgtggggagcaaacaggattagataccctggtagtccacgccgtaaacgatgaatactaggtgtcgggg |
| | | gacaacgtccttcggtgccgccgctaacgcaataagtattccacctggggagtacgttcgcaagaatgaaactcaaaggaatt |
| | | gacggggacccgcacaagcggtggagcatgtggtttaattcgaagcaacgcgaagaaccttaccaagtcttgacatcccattg |
| | | aaaacactttaaccggtgtccctcttcggagcaatggagacaggtggtgcatggttgtcgtcagctcgtgtcgtgagatgttggg |
| | | ttaagtcccgcaacgagcgcaacccttatccttagtagccagcacatgatggtgggcactctggggagactgccagggataac |
| | | ctggaggaaggtggggatgacgtcaaatcatcatgccccttatgatttgggctacacacgtgctacaatggcgtaaacaaagg |
| | | gaagcaaaggagcgatctggagcaaaccccaaaaataacgtctcagttcggattgcaggctgcaactcgcctgcatgaagct |
| | | ggaatcgctagtaatcgcgaatcagaatgtcgcggtgaatacgttcccgggtcttgtacacaccgcccgtcacaccatgggag |
| | | ttggtaacgcccgaagtcagtgacccaaccgcaaggagggagctgccgaaggcgggactgataactggggtgaagtcgta |
| | | acaaggtagccgtatcggaaggtgcggctggatcacctccttt |
| Clostridium_ | SEQ ID | atgagagttcgatcctggctcaggatgaacgctggcggcgtgcttaacacatgcaagtcgagcgaagcggtttcaatgaagttt |
| hathewayi | NO: 230 | tcggatggatttgaaattgacttagcggcggacgggtgagtaacgcgtgggtaacctgccttacactgggggataacagttag |
| | | aaatgactgctaataccgcataagcgcacagggccgcatggtctggtgtgaaaaactccggtggtgtaagatggacccgcgt |
| | | ctgattaggtagttggtggggtaacggcccaccaagccgacgatcagtagccgacctgagagggtgaccggccacattggg |
| | | actgagacacggcccaaactcctacgggaggcagcagtggggaatattggacaatgggcgaaagcctgatccagcgacgc |
| | | cgcgtgagtgaagaagtatttcggtatgtaaagctctatcagcagggaagaaaatgacggtacctgactaagaagccccggct |
| | | aactacgtgccagcagccgcggtaatacgtagggggcaagcgttatccggatttactgggtgtaaagggagcgtagacggtt |
| | | aagcaagtctgaagtgaaagcccggggctcaaccccggtactgctttggaaactgtttgacttgagtgcaggagaggtaagtg |
| | | gaattcctagtgtagcggtgaaatgcgtagatattaggaggaacaccagtggcgaaggcggcttactggactgtaactgacgtt |
| | | gaggctcgaaagcgtggggagcaaacaggattagataccctggtagtccacgccgtaaacgatgaatactaggtgtcgggg |
| | | gacaaagtccttcggtgccgccgctaacgcaataagtattccacctggggagtacgttcgcaagaatgaaactcaaaggaatt |
| | | gacggggacccgcacaagcgg |
| Clostridium_ | SEQ ID | atgagagtttgatcctggctcaggatgaacgctggcggcgtgcctaacacatgcaagtcgaacgaagcaattaaaggaagtttt |
| clostridioforme | NO: 231 | cggacggaatttgattgactgagtggcggacgggtgagtaacgcgtggataacctgcctcacactgggggataacagttaga |
| | | aatgactgctaataccgcataagcgcacagtaccgcatggtacggtgtgaaaaactccggtggtgtgagatggatccgcgtct |
| | | gattagccagttggcggggtaacggcccaccaaagcgacgatcagtagccgacctgagagggtgaccggccacattggga |
| | | ctgagacacggcccaaactcctacgggaggcagcagtggggaatattgcacaatgggcgaaagcctgatgcagcgacgcc |
| | | gcgtgagtgaagaagtatttcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgtagacggcg |
| | | aagcaagtctgaagtgaaaacccagggctcaaccctgggactgctttggaaactgttttgctagagtgtcggagaggtaagtg |
| | | gaattcctagtgtagcggtgaaatgcgtagatattaggaggaacaccagtggcgaaggcggcttactggacgataactgacgt |
| | | tgaggctcgaaagcgtggggagcaaacaggattagataccctggtagtccacgccgtaaacgatgaatgctaggtgttgggg |
| | | ggcaaagcccttcggtgccgtcgcaaacgcagtaagcattccacctggggagtacgttcgcaagaatgaaactcaaaggaatt |
| | | gacggggacccgcacaagcggtggagcatgtggtttaattcgaagcaacgcgaagaaccttaccaagtcttgacatccttttg |
| | | accggcgtgtaacggcgccttcccttcggggcaagagagacaggtggtgcatggttgtcgtcagctcgtgtcgtgagatgttg |
| | | ggttaagtcccgcaacgagcgcaacccttatccttagtagccagcaggtagagctgggcactctagggagactgccagggat |
| | | aacctggaggaaggcggggatgacgtcaaatcatcatgccccttatgatttgggctacacacgtgctacaatggcgtaaacaa |
| | | agggaagcaagacagtgatgtggagcaaatcccaaaaataacgtcccagttcggactgtagtctgcaacccgactacacgaa |
| | | gctggaatcgctagtaatcgcgaatcagaatgtcgcggtgaatacgttcccgggtcttgtacacaccgcccgtcacaccatgg |
| | | gagtcagcaacgcccgaagtcagtgacccaactcgcaagagagggagctgccgaaggcggggcaggtaactggggtgaa |
| | | gtcgtaacaaggtagccgtatcggaaggtgcggctggatcacctccttt |
| Clostridium_ | SEQ ID | atgagagtttgatcctggctcaggatgaacgctggcggcgtgcctaacacatgcaagtcgaacgaagcaattaaaggaagtttt |
| clostridioforme | NO: 232 | cggatggaatttgattgactgagtggcggacgggtgagtaacgcgtggataacctgcctcacactgggggataacagttagaa |
| | | atgactgctaataccgcataagcgcacagtgccgcatggtacggtgtgaaaaactccggtggtgtgagatggatccgcgtctg |
| | | attagccagaggcggggtaacggcccaccaaagcgacgatcagtagccgacctgagagggtgaccggccacattgggact |
| | | gagacacggcccaaactcctacgggaggcagcagtggggaatattgcacaatgggcgaaagcctgatgcagcgacgccgc |
| | | gtgagtgaagaagtatttcggtatgtaaagctctatcagcagggaagaaaatgacggtacctgactaagaagccccggctaac |
| | | tacgtgccagcagccgcggtaatacgtagggggcaagcgttatccggatttactgggtgtaaagggagcgtagacggcgaa |
| | | gcaagtctgaagtgaaaacccagggctcaaccctgggactgctttggaaactgttttgctagagtgtcggagaggtaagtgga |
| | | attcctagtgtagcggtgaaatgcgtagatattaggaggaacaccagtggcgaaggcggcttactggacgataactgacgttg |
| | | aggctcgaaagcgtggggagcaaacaggattagataccctggtagtccacgccgtaaacgatgaatgctaggtgttggggg |
| | | gcaaagcccttcggtgccgtcgcaaacgcagtaagcattccacctggggagtacgttcgcaagaatgaaactcaaaggaattg |
| | | acggggacccgcacaagcggtggagcatgtggtttaattcgaagcaacgcgaagaaccttaccaagtcttgacatcctcttga |
| | | ccggcgtgtaacggcgccttcccttcggggcaagagagacaggtggtgcatggttgtcgtcagctcgtgtcgtgagatgttgg |
| | | gttaagtcccgcaacgagcgcaacccttatccttagtagccagcaggtagagctgggcactctagggagactgccagggata |
| | | acctggaggaaggtggggatgacgtcaaatcatcatgccccttatgatttgggctacacacgtgctacaatggcgtaaacaaa |
| | | gggaagcaagacagtgatgtggagcaaatcccaaaaataacgtcccagttcggactgtagtctgcaacccgactacacgaag |
| | | ctggaatcgctagtaatcgcgaatcagaatgtcgcggtgaatacgttcccgggtcttgtacacaccgcccgtcacaccatggga |
| | | gtcagcaacgcccgaagtcagtgacccaactcgcaagagagggagctgccgaaggcggggcaggtaactggggtgaagt |
| | | cgtaacaaggtagccgtatcggaaggtgcggctggatcacctccttt |
| Clostridium_ | SEQ ID | atgagagtttgatcctggctcaggatgaacgctggcggcgtgcctaacacatgcaagtcgaacgaagcaattaaaggaagtttt |
| clostridioforme | NO: 233 | cggatggaatttgattgactgagtggcggacgggtgagtaacgcgtggataacctgcctcacactgggggataacagttagaa |
| | | atgactgctaataccgcataagcgcacagtaccgcatggtacggtgtgaaaaactccggtggtgtgagatggatccgcgtctg |
| | | attagccagttggcggggtaacggcccaccaaagcgacgatcagtagccgacctgagagggtgaccggccacattgggact |
| | | gagacacggcccaaactcctacgggaggcagcagtggggaatattgcacaatgggcgaaagcctgatgcagcgacgccgc |
| | | gtgagtgaagaagtatttcggtatgtaaagctctatcagcagggaagaaaatgacggtacctgactaagaagccccggctaac |
| | | tacgtgccagcagccgcggtaatacgtagggggcaagcgttatccggatttactgggtgtaaagggagcgtagacggcgaa |
| | | gcaagtctgaagtgaaaacccagggctcaaccctgggactgctttggaaactgttttgctagagtgtcggagaggtaagtgga |
| | | attcctagtgtagcggtgaaatgcgtagatattaggaggaacaccagtggcgaaggcggcttactggacgataactgacgttg |
| | | aggctcgaaagcgtggggagcaaacaggattagataccctggtagtccacgccgtaaacgatgaatgctaggtgttggggg |
| | | gcaaagcccttcggtgccgtcgcaaacgcagtaagcattccacctggggagtacgttcgcaagaatgaaactcaaaggaattg |
| | | acggggacccgcacaagcggtggagcatgtggtttaattcgaagcaacgcgaagaaccttaccaagtcttgacatcctcttgg |
| | | ccggcgtgtaacggcgccttcccttcggggcaagagagacaggtggtgcatggttgtcgtcagctcgtgtcgtgagatgttgg |
| | | gttaagtcccgcaacgagcgcaacccttatccttagtagccagcaggtagagctgggcactctagggagactgccagggata |
| | | acctggaggaaggtggggatgacgtcaaatcatcatgccccttatgatttgggctacacacgtgctacaatggcgtaaacaaa |
| | | gggaagcaagacagtgatgtggagcaaatcccaaaaataacgtcccagttcggactgtagtctgcaacccgactacacgaag |
| | | ctggaatcgctagtaatcgcgaatcagaatgtcgcggtgaatacgttcccgggtcttgtacacaccgcccgtcacaccatggga |
| | | gtcagcaacgcccgaagtcagtgacccaactcgcaagagagggagctgccgaaggcggggcaggtaactggggtgaagt |
| | | cgtaacaaggtagccgtatcggaaggtgcggctggatcacctccttt |
| Lactobacillus_ | SEQ ID | tatgagagtttgatcctggctcaggatgaacgccggcggtgtgcctaatacatgcaagtcgaacgcgttggcccgattgattgat |
| fermentum | NO: 234 | ggtgcttgcacctgattgattttggtcgccaacgagtggcggacgggtgagtaacacgtaggtaacctgcccagaagcgggg |
| | | gacaacatttggaaacagatgctaataccgcataacaacgttgttcgcatgaacaacgcttaaaagatggcttctcgctatcactt |
| | | ctggatggacctgcggtgcattagcttgttggtggggtaacggcctaccaaggcgatgatgcatagccgagttgagagactga |
| | | tcggccacaatgggactgagacacggcccatactcctacgggaggcagcagtagggaatcttccacaatgggcgcaagcct |
| | | gatggagcaacaccgcgtgagtgaagaagggtttcggctcgtaaagctctgttgttaaagaagaacacgtatgagagtaactg |
| | | ttcatacgttgacggtatttaaccagaaagtcacggctaactacgtgccagcagccgcggtaatacgtaggtggcaagcgttat |
| | | ccggatttattgggcgtaaagagagtgcaggcggttttctaagtctgatgtgaaagccttcggcttaaccggagaagtgcatcg |
| | | gaaactggataacttgagtgcagaagagggtagtggaactccatgtgtagcggtggaatgcgtagatatatggaagaacacca |
| | | gtggcgaaggcggctacctggtctgcaactgacgctgagactcgaaagcatgggtagcgaacaggattagataccctggtag |
| | | tccatgccgtaaacgatgagtgctaggtgttggagggtttccgcccttcagtgccggagctaacgcattaagcactccgcctgg |
| | | ggagtacgaccgcaaggttgaaactcaaaggaattgacgggggcccgcacaagcggtggagcatgtggtttaattcgaagct |
| | | acgcgaagaaccttaccaggtcttgacatcttgcgccaaccctagagatagggcgtttccttcgggaacgcaatgacaggtgg |
| | | tgcatggtcgtcgtcagctcgtgtcgtgagatgttgggttaagtcccgcaacgagcgcaacccttgttactagttgccagcattaa |
| | | gttgggcactctagtgagactgccggtgacaaaccggaggaaggtggggacgacgtcagatcatcatgccccttatgacctg |
| | | ggctacacacgtgctacaatggacggtacaacgagtcgcgaactcgcgagggcaagcaaatctcttaaaaccgttctcagttc |
| | | ggactgcaggctgcaactcgcctgcacgaagtcggaatcgctagtaatcgcggatcagcatgccgcggtgaatacgttcccg |
| | | ggccttgtacacaccgcccgtcacaccatgagagtttgtaacacccaaagtcggtggggtaaccttttaggagccagccgcct |
| | | aaggtgggacagatgattagggtgaagtcgtaacaaggtagccgtaggagaacctgcggctggatcacctccttt |
| Lactobacillus_ | SEQ ID | tatgagagtttgatcctggctcaggatgaacgccggcggtgtgcctaatacatgcaagtcgaacgcgttggcccaattgattga |
| fermentum | NO: 235 | cggtgcttgcacctgattgattttggtcgccaacgagtggcggacgggtgagtaacacgtaggtaacctgcccagaagcggg |
| | | ggacaacatttggaaacagatgctaataccgcataacaacgttgttcgcatgaacaacgcttaaaagatggcttctcgctatcact |
| | | tctggatggacctgcggtgcattagcttgttggtggggtaacggcctaccaaggcgatgatgcatagccgagttgagagactg |
| | | atcggccacaatgggactgagacacggcccatactcctacgggaggcagcagtagggaatcttccacaatgggcgcaagcc |
| | | tgatggagcaacaccgcgtgagtgaagaagggtttcggctcgtaaagctctgttgttaaagaagaacacgtatgagagtaact |
| | | gttcatacgttgacggtatttaaccagaaagtcacggctaactacgtgccagcagccgcggtaatacgtaggtggcaagcgtta |
| | | tccggatttattgggcgtaaagagagtgcaggcggttttctaagtctgatgtgaaagccttcggcttaaccggagaagtgcatcg |
| | | gaaactggataacttgagtgcagaagagggtagtggaactccatgtgtagcggtggaatgcgtagatatatggaagaacacca |
| | | gtggcgaaggcggctacctggtctgcaactgacgctgagactcgaaagcatgggtagcgaacaggattagataccctggtag |
| | | tccatgccgtaaacgatgagtgctaggtgttggagggtttccgcccttcagtgccggagctaacgcattaagcactccgcctgg |
| | | ggagtacgaccgcaaggttgaaactcaaaggaattgacgggggcccgcacaagcggtggagcatgtggtttaattcgaagct |
| | | acgcgaagaaccttaccaggtcttgacatcttgcgccaaccctagagatagggcgtttccttcgggaacgcaatgacaggtgg |
| | | tgcatggtcgtcgtcagctcgtgtcgtgagatgttgggttaagtcccgcaacgagcgcaacccttgttactagttgccagcattaa |
| | | gttgggcactctagtgagactgccggtgacaaaccggaggaaggtggggacgacgtcagatcatcatgccccttatgacctg |
| | | ggctacacacgtgctacaatggacggtacaacgagtcgcgaactcgcgagggcaagcaaatctcttaaaaccgttctcagttc |
| | | ggactgcaggctgcaactcgcctgcacgaagtcggaatcgctagtaatcgcggatcagcatgccgcggtgaatacgttcccg |
| | | ggccttgtacacaccgcccgtcacaccatgagagtttgtaacacccaaagtcggtggggtaaccttttaggagccagccgcct |
| | | aaggtgggacagatgattagggtgaagtcgtaacaaggtagccgtaggagaacctgcggctggatcacctccttt |
| Lactobacillus_ | SEQ ID | tatgagagtttgatcctggctcaggatgaacgccggcggtgtgcctaatacatgcaagtcgaacgcgttggcccaattgattgat |
| fermentum | NO: 236 | ggtgcttgcacctgattgattttggtcgccaacgagtggcggacgggtgagtaacacgtaggtaacctgcccagaagcgggg |
| | | gacaacatttggaaacagatgctaataccgcataacaacgttgttcgcatgaacaacgcttaaaagatggcttctcgctatcactt |
| | | ctggatggacctgcggtgcattagcttgttggtggggtaacggcctaccaaggcgatgatgcatagccgagttgagagactga |
| | | tcggccacaatgggactgagacacggcccatactcctacgggaggcagcagtagggaatcttccacaatgggcgcaagcct |
| | | gatggagcaacaccgcgtgagtgaagaagggtttcggctcgtaaagctctgttgttaaagaagaacacgtatgagagtaactg |
| | | ttcatacgttgacggtatttaaccagaaagtcacggctaactacgtgccagcagccgcggtaatacgtaggtggcaagcgttat |
| | | ccggatttattgggcgtaaagagagtgcaggcggttttctaagtctgatgtgaaagccttcggcttaaccggagaagtgcatcg |
| | | gaaactggataacttgagtgcagaagagggtagtggaactccatgtgtagcggtggaatgcgtagatatatggaagaacacca |
| | | gtggcgaaggcggctacctggtctgcaactgacgctgagactcgaaagcatgggtagcgaacaggattagataccctggtag |
| | | tccatgccgtaaacgatgagtgctaggtgttggagggtttccgcccttcagtgccggagctaacgcattaagcactccgcctgg |
| | | ggagtacgaccgcaaggttgaaactcaaaggaattgacgggggcccgcacaagcggtggagcatgtggtttaattcgaagct |
| | | acgcgaagaaccttaccaggtcttgacatcttgcgccaaccctagagatagggcgtttccttcgggaacgcaatgacaggtgg |
| | | tgcatggtcgtcgtcagctcgtgtcgtgagatgttgggttaagtcccgcaacgagcgcaacccttgttactagttgccagcattaa |
| | | gttgggcactctagtgagactgccggtgacaaaccggaggaaggtggggacgacgtcagatcatcatgccccttatgacctg |
| | | ggctacacacgtgctacaatggacggtacaacgagtcgcgaactcgcgagggcaagcaaatctcttaaaaccgttctcagttc |
| | | ggactgcaggctgcaactcgcctgcacgaagtcggaatcgctagtaatcgcggatcagcatgccgcggtgaatacgttcccg |
| | | ggccttgtacacaccgcccgtcacaccatgagagtttgtaacacccaaagtcggtggggtaaccttttaggagccagccgcct |
| | | aaggtgggacagatgattagggtgaagtcgtaacaaggtagccgtaggagaacctgcggctggatcacctccttt |
| Lactobacillus_ | SEQ ID | tatgagagtttgatcctggctcaggatgaacgccggcggtgtgcctaatacatgcaagtcgaacgcgttggtccaattgattgac |
| fermentum | NO: 237 | ggtgcttgcacctgattgattttggtcgccaacgagtggcggacgggtgagtaacacgtaggtaacctgcccagaagcgggg |
| | | gacaacatttggaaacagatgctaataccgcataacaacgttgttcgcatgaacaacgtttaaaagatggcttctcgctatcactt |
| | | ctggatggacctgcggtgcattagcttgaggtggggtaatggcctaccaaggcgatgatgcatagccgagttgagagactgat |
| | | cggccacaatgggactgagacacggcccatactcctacgggaggcagcagtagggaatcttccacaatgggcgcaagcctg |
| | | atggagcaacaccgcgtgagtgaagaagggtttcggctcgtaaagctctgttgttaaagaagaacacgtatgagagtaactgtt |
| | | catacgttgacggtatttaaccagaaagtcacggctaactacgtgccagcagccgcggtaatacgtaggtggcaagcgttatcc |
| | | ggatttattgggcgtaaagagagtgcaggcggttttctaagtctgatgtgaaagccttcggcttaaccggagaagtgcatcgga |
| | | aactggataacttgagtgcagaagagggtagtggaactccatgtgtagcggtggaatgcgtagatatatggaagaacaccagt |
| | | ggcgaaggcggctacctggtctgcaactgacgctgagactcgaaagcatgggtagcgaacaggattagataccctggtagtc |
| | | catgccgtaaacgatgagtgctaggtgttggagggtttccgcccttcagtgccggagctaacgcattaagcactccgcctggg |
| | | gagtacgaccgcaaggttgaaactcaaaggaattgacgggggcccgcacaagcggtggagcatgtggtttaattcgaagcta |
| | | cgcgaagaaccttaccaggtcttgacatcttgcgccaaccctagagatagggcgtttccttcgggaacgcaatgacaggtggt |
| | | gcatggtcgtcgtcagctcgtgtcgtgagatgttgggttaagtcccgcaacgagcgcaacccttgttactagttgccagcattaa |
| | | gttgggcactctagtgagactgccggtgacaaaccggaggaaggtggggacgacgtcagatcatcatgccccttatgacctg |
| | | ggctacacacgtgctacaatggacggtacaacgagtcgcaaactcgcgagggcaagcaaatctcttaaaaccgttctcagttc |
| | | ggactgcaggctgcaactcgcctgcacgaagtcggaatcgctagtaatcgcggatcagcatgccgcggtgaatacgttcccg |
| | | ggccttgtacacaccgcccgtcacaccatgagagtttgtaacacccaaagtcggtggggtaaccttttaggagccagccgcct |
| | | aaggtgggacagatgattagggtgaagtcgtaacaaggtagccgtaggagaacctgcggctggatcacctcctt |
| Lactobacillus_ | SEQ ID | tatgagagtttgatcctggctcaggatgaacgccggcggtgtgcctaatacatgcaagtcgaacgcgttggcccaattgattgat |
| fermentum | NO: 238 | ggtgcttgcacctgattgattttggtcgccaacgagtggcggacgggtgagtaacacgtaggtaacctgcccagaagcgggg |
| | | gacaacatttggaaacagatgctaataccgcataacaacgttgttcgcatgaacaacgcttaaaagatggcttctcgctatcactt |
| | | ctggatggacctgcgctgcattagcttgttggtggggtaatggcctaccaaggcgatgatgcatagccgagttgagagactgat |
| | | cggccacaatgggactgagacacggcccatactcctacgggaggcagcagtagggaatcttccacaatgggcgcaagcctg |
| | | atggagcaacaccgcgtgagtgaagaagggtttcggctcgtaaagctctgttgttaaagaagaacacgtatgagagtaactgtt |
| | | catacgttgacggtatttaaccagaaagtcacggctaactacgtgccagcagccgcggtaatacgtaggtggcaagcgttatcc |
| | | ggatttattgggcgtaaagagagtgcaggcggttttctaagtctgatgtgaaagccttcggcttaaccggagaagtgcatcgga |
| | | aactggataacttgagtgcagaagagggtagtggaactccatgtgtagcggtggaatgcgtagatatatggaagaacaccagt |
| | | ggcgaaggcggctacctggtctgcaactgacgctgagactcgaaagcatgggtagcgaacaggattagataccctggtagtc |
| | | catgccgtaaacgatgagtgctaggtgttggagggtttccgcccttcagtgccggagctaacgcattaagcactccgcctggg |
| | | gagtacgaccgcaaggttgaaactcaaaggaattgacgggggcccgcacaagcggtggagcatgtggtttaattcgaagcta |
| | | cgcgaagaaccttaccaggtcttgacatcttgcgccaaccctagagatagggcgtttccttcgggaacgcaatgacaggtggt |
| | | gcatggtcgtcgtcagctcgtgtcgtgagatgttgggttaagtcccgcaacgagcgcaacccttgttactagttgccagcattaa |
| | | gttgggcactctagtgagactgccggtgacaaaccggaggaaggtggggacgacgtcagatcatcatgccccttatgacctg |
| | | ggctacacacgtgctacaatggacggtacaacgagtcgcgaactcgtgagggcaagcaaatctcttaaaaccgttctcagttc |
| | | ggactgcaggctgcaactcgcctgcacgaagtcggaatcgctagtaatcgcggatcagcatgccgcggtgaatacgttcccg |
| | | ggccttgtacacaccgcccgtcacaccatgagagtttgtaacacccaaagtcggtggggtaaccttttaggagccagccgcct |
| | | aaggtgggacagatgattagggtgaagtcgtaacaaggtagccgtaggagaacctgcggctggatcacctcctt |
| Bacteroides_ | SEQ ID | ctgaaccagccaagtagcgtgaaggatgactgccctatgggttgtaaacttcttttatatgggaataaagttttccacgtgtggaat |
| thetaiotaomicron | NO: 239 | tttgtatgtaccatatgaataaggatcggctaactccgtgccagcagccgcggtaatacggaggatccgagcgttatccggattt |
| | | attgggtttaaagggagcgtaggtggacagttaagtcagttgtgaaagtttgcggctcaaccgtaaaattgcagttgatactggct |
| | | gtcttgagtacagtagaggtgggcggaattcgtggtgtagcggtgaaatgcttagatatcacgaagaactccgattgcgaagg |
| | | cagctcactggactgcaactgacactgatgctcgaaagtgtgggtatcaaacaggattagataccctggtagtccacacagtaa |
| | | acgatgaatactcgctgtttgcgatatacagtaagcggccaagcgaaagcattaagtattccacctggggagtacgccggcaa |
| | | cggtgaaactcaaaggaattgacgggggcccgcacaagcggaggaacatgtggtttaattcgatgatacgcgaggaacctta |
| | | cccgggcttaaattgcatttgaatatattggaaacagtatagccgtaaggcaaatgtgaaggtgctgcatggttgtcgtcagctcg |
| | | tgccgtgaggtgtcggcttaagtgccataacgagcgcaacccttatctttagttactaacaggtcatgctgaggactctagagag |
| | | actgccgtcgtaagatgtgaggaaggtggggatgacgtcaaatcagcacggcccttacgtccggggctacacacgtgttaca |
| | | atggggggtacagaaggcagctacctggtgacaggatgctaatcccaaaagcctctctcagttcggatcgaagtctgcaaccc |
| | | gacttcgtgaagctggattcgctagtaatcgcgcatcagccatggcgcggtgaatacgttcccgggccttgtacacaccgccc |
| | | gtcaagccatgaaagccgggggtacctgaagtacgtaaccgcaaggagcgtcctagggtaaaactggtaattggggctaagt |
| | | cgtaacaaggtagccgtaccggaaggtgcggctggaacacctcctt |
| Bacteroides_ | SEQ ID | ctgaaccagccaagtagcgtgaaggatgactgccctatgggttgtaaacttcttttatatgggaataaagttttccacgtgtggaat |
| thetaiotaomicron | NO: 240 | tttgtatgtaccatatgaataaggatcggctaactccgtgccagcagccgcggtaatacggaggatccgagcgttatccggattt |
| | | attgggtttaaagggagcgtaggtggacagttaagtcagttgtgaaagtttgcggctcaaccgtaaaattgcagttgatactggct |
| | | gtcttgagtacagtagaggtgggcggaattcgtggtgtagcggtgaaatgcttagatatcacgaagaactccgattgcgaagg |
| | | cagctcactggactgcaactgacactgatgctcgaaagtgtgggtatcaaacaggattagataccctggtagtccacacagtaa |
| | | acgatgaatactcgctgtttgcgatatacagtaagcggccaagcgaaagcattaagtattccacctggggagtacgccggcaa |
| | | cggtgaaactcaaaggaattgacgggggcccgcacaagcggaggaacatgtggtttaattcgatgatacgcgaggaacctta |
| | | cccgggcttaaattgcatttgaatatattggaaacagtatagccgtaaggcaaatgtgaaggtgctgcatggttgtcgtcagctcg |
| | | tgccgtgaggtgtcggcttaagtgccataacgagcgcaacccttatctttagttactaacaggtcatgctgaggactctagagag |
| | | actgccgtcgtaagatgtgaggaaggtggggatgacgtcaaatcagcacggcccttacgtccggggctacacacgtgttaca |
| | | atggggggtacagaaggcagctacctggtgacaggatgctaatcccaaaagcctctctcagttcggatcgaagtctgcaaccc |
| | | gacttcgtgaagctggattcgctagtaatcgcgcatcagccatggcgcggtgaatacgttcccgggccttgtacacaccgccc |
| | | gtcaagccatgaaagccgggggtacctgaagtacgtaaccgcaaggagcgtcctagggtaaaactggtaattggggctaagt |
| | | cgtaacaaggtagccgtaccggaaggtgcggctggaacacctcctt |
| Bacteroides_ | SEQ ID | atgaagagtttgatcctggctcaggatgaacgctagctacaggcttaacacatgcaagtcgaggggcagcatttcagtttgcttg |
| thetaiotaomicron | NO: 241 | caaactggagatggcgaccggcgcacgggtgagtaacacgtatccaacctgccgataactcggggatagcctttcgaaagaa |
| | | agattaatacccgatagtataattagaccgcatggtcttattattaaagaatttcggttatcgatggggatgcgttccattaggcagt |
| | | tggtgaggtaacggctcaccaaaccttcgatggataggggttctgagaggaaggtcccccacattggaactgagacacggtc |
| | | caaactcctacgggaggcagcagtgaggaatattggtcaatggacgagagtctgaaccagccaagtagcgtgaaggatgact |
| | | gccctatgggttgtaaacttcttttatatgggaataaagttttccacgtgtggaattttgtatgtaccatatgaataaggatcggctaa |
| | | ctccgtgccagcagccgcggtaatacggaggatccgagcgttatccggatttattgggtttaaagggagcgtaggtggacagt |
| | | taagtcagttgtgaaagtttgcggctcaaccgtaaaattgcagttgatactggctgtcttgagtacagtagaggtgggcggaattc |
| | | gtggtgtagcggtgaaatgcttagatatcacgaagaactccgattgcgaaggcagctcactggactgcaactgacactgatgct |
| | | cgaaagtgtgggtatcaaacaggattagataccctggtagtccacacagtaaacgatgaatactcgctgtttgcgatatacagta |
| | | agcggccaagcgaaagcattaagtattccacctggggagtacgccggcaacggtgaaactcaaaggaattgacgggggcc |
| | | cgcacaagcggaggaacatgtggtttaattcgatgatacgcgaggaaccttacccgggcttaaattgcatttgaatatattggaa |
| | | acagtatagccgtaaggcaaatgtgaaggtgctgcatggttgtcgtcagctcgtgccgtgaggtgtcggcttaagtgccataac |
| | | gagcgcaacccttatctttagttactaacaggtcatgctgaggactctagagagactgccgtcgtaagatgtgaggaaggtggg |
| | | gatgacgtcaaatcagcacggcccttacgtccggggctacacacgtgttacaatggggggtacagaaggcagctacctggtg |
| | | acaggatgctaatcccaaaagcctctctcagttcggatcgaagtctgcaacccgacttcgtgaagctggattcgctagtaatcgc |
| | | gcatcagccatggcgcggtgaatacgttcccgggccttgtacacaccgcccgtcaagccatgaaagccgggggtacctgaa |
| | | gtacgtaaccgcaaggagcgtcctagggtaaaactggtaattggggctaagtcgtaacaaggtagccgtaccggaaggtgcg |
| | | gctggaacacctcctt |
| Bacteroides_ | SEQ ID | atgaagagtttgatcctggctcaggatgaacgctagctacaggcttaacacatgcaagtcgaggggcagcatttcagtttgcttg |
| thetaiotaomicron | NO: 242 | caaactggagatggcgaccggcgcacgggtgagtaacacgtatccaacctgccgataactcggggatagcctttcgaaagaa |
| | | agattaatacccgatggtataattagaccgcatggtcttgttattaaagaatttcggttatcgatggggatgcgttccattaggcagt |
| | | tggtgaggtaacggctcaccaaaccttcgatggataggggttctgagaggaaggtcccccacattggaactgagacacggtc |
| | | caaactcctacgggaggcagcagtgaggaatattggtcaatgggcgcaggcctgaaccagccaagtagcgtgaaggatgac |
| | | tgccctatgggttgtaaacttcttttatatgggaataaagttttccacgtgtggattttagtatgtaccatatgaataaggatcggcta |
| | | actccgtgccagcagccgcggtaatacggaggatccgagcgttatccggatttattgggtttaaagggagcgtaggtggacag |
| | | ttaagtcagttgtgaaagtttgcggctcaaccgtaaaattgcagttgatactggctgtcttgagtacagtagaggtgggcggaatt |
| | | cgtggtgtagcggtgaaatgcttagatatcacgaagaactccgattgcgaaggcagctcactggactgcaactgacactgatg |
| | | ctcgaaagtgtgggtatcaaacaggattagataccctggtagtccacacagtaaacgatgaatactcgctgtttgcgatatacag |
| | | taagcggccaagcgaaagcattaagtattccacctggggagtacgccggcaacggtgaaactcaaaggaattgacgggggc |
| | | ccgcacaagcggaggaacatgtggtttaattcgatgatacgcgaggaaccttacccgggcttaaattgcatttgaatatattgga |
| | | aacagtatagccgtaaggcaaatgtgaaggtgctgcatggttgtcgtcagacgtgccgtgaggtgtcggcttaagtgccataa |
| | | cgagcgcaacccttatctttagttactaacaggtcatgctgaggactctagagagactgccgtcgtaagatgtgaggaaggtgg |
| | | ggatgacgtcaaatcagcacggcccttacgtccggggctacacacgtgttacaatggggggtacagaaggcagctacctggt |
| | | gacaggatgctaatcccaaaagcctctctcagttcggatcgaagtctgcaacccgacttcgtgaagaggattcgctagtaatcg |
| | | cgcatcagccatggcgcggtgaatacgttcccgggccttgtacacaccgcccgtcaagccatgaaagccgggggtacctgaa |
| | | gtacgtaaccgcaaggagcgtcctagggtaaaactggtaattggggctaagtcgtaacaaggtagccgtaccggaaggtgcg |
| | | gctggaacacctcctt |
| Sutterella_ | SEQ ID | taaagagtttgatcctggctcagattgaacgctggcggcatgctttacacatgcaagtcgaacggcagcgcggggagcttgct |
| wadsworthensis | NO: 243 | ccctggcggcgagtggcgcacgggtgagtaatacatcggaacgtgtcttctagtgggggataactgcccgaaagggcagct |
| | | aataccgcatgagacctgagggtgaaagcgggggatcgcaagacctcgcgctggaagagcggccgatgtccgattagcta |
| | | gttggtgaggtaaaggctcaccaaggcgacgatcggtagctggtctgagaggacgaccagccacactgggactgagacac |
| | | ggcccagactcctacgggaggcagcagtggggaattttggacaatgggggcaaccctgatccagccatgccgcgtgcagg |
| | | atgaaggtcttcggattgtaaactgcttttgtcagggacgaaaagggatgcgataacaccgtattccgctgacggtacctgaag |
| | | aataagcaccggctaactacgtgccagcagccgcggtaatacgtagggtgcaagcgttaatcggaattactgggcgtaaagc |
| | | gtgcgcaggcggttctgtaagatagatgtgaaatccccgggctcaacctgggaattgcatatatgactgcaggacttgagtttgt |
| | | cagaggagggtggaattccacgtgtagcagtgaaatgcgtagatatgtggaagaacaccgatggcgaaggcagccctctgg |
| | | ggcatgactgacgctcatgcacgaaagcgtggggagcaaacaggattagataccctggtagtccacgccctaaacgatgtct |
| | | actagttgttggggacgatagtccttggtaacgcagctaacgcgtgaagtagaccgcctggggagtacggtcgcaagattaaa |
| | | actcaaaggaattgacggggacccgcacaagcggtggatgatgtggattaattcgatgcaacgcgaaaaaccttacctagcct |
| | | tgacatgccaggaaggtctgagagatcagaccgtgcccgcaagggaatctggacacaggtgctgcatggctgtcgtcagctc |
| | | gtgtcgtgagatgttgggttaagtcccgcaacgagcgcaacccttgtcattagagctacgaaagggcactctaatgagactgc |
| | | cggtgacaaaccggaggaaggtggggatgacgtcaagtcctcatggcccttatggctagggcctcacacgtcatacaatggt |
| | | cggaacagagggaagcgaagccgcgaggtgaagccaatcccagaaaaccgatcgtagtccggattgcagtctgcaactcg |
| | | actgcatgaagtcggaatcgctagtaatcgcggatcagcatgccgcggtgaatacgttcccgggtcttgtacacaccgcccgt |
| | | cacaccatgggagtggggttcaccagaagacgtttgcccaaccgaaaggaaggcggcgtccacggtgggcttcatgactgg |
| | | ggtgaagtcgtaacaaggtagccgtaccggaaggtgcggctggatcacctccttt |
| Sutterella_ | SEQ ID | taaagagtttgatcctggctcagattgaacgctggcggcatgctttacacatgcaagtcgaacggcagcgcggggagcttgct |
| wadsworthensis | NO: 244 | ccctggcggcgagtggcgcacgggtgagtaatacatcggaacgtgtcttctagtgggggataactgcccgaaagggcagct |
| | | aataccgcatgagacctgagggtgaaagcgggggatcgcaagacctcgcgctggaagagcggccgatgtccgattagcta |
| | | gttggtgaggtaaaggctcaccaaggcgacgatcggtagctggtctgagaggacgaccagccacactgggactgagacac |
| | | ggcccagactcctacgggaggcagcagtggggaattttggacaatgggggcaaccctgatccagccatgccgcgtgcagg |
| | | atgaaggtcttcggattgtaaactgcttttgtcagggacgaaaagggatgcgataacaccgtattccgctgacggtacctgaag |
| | | aataagcaccggctaactacgtgccagcagccgcggtaatacgtagggtgcaagcgttaatcggaattactgggcgtaaagc |
| | | gtgcgcaggcggttctgtaagatagatgtgaaatccccgggctcaacctgggaattgcatatatgactgcaggacttgagtttgt |
| | | cagaggagggtggaattccacgtgtagcagtgaaatgcgtagatatgtggaagaacaccgatggcgaaggcagccctctgg |
| | | gacatgactgacgctcatgcacgaaagcgtggggagcaaacaggattagataccctggtagtccacgccctaaacgatgtcta |
| | | ctagttgttggggacgatagtccttggtaacgcagctaacgcgtgaagtagaccgcctggggagtacggtcgcaagattaaaa |
| | | ctcaaaggaattgacgggn |
| Sutterella_ | SEQ ID | taaagagtttgatcctggctcagattgaacgctggcggctttgctttacacatgcaagtcgaacggcagcgcggggagcttgct |
| wadsworthensis | NO: 245 | ccctggcggcgagtggcgcacgggtgagtaatacatcggaacgtgtcttctagtgggggataactgtccgaaagggcagcta |
| | | ataccgcatgagacctgagggtgaaagcgggggatcgcaagacctcgcgctggaagagcggccgatgtccgattagctagt |
| | | tggtgaggtaaaggctcaccaaggcgacgatcggtagctggtctgagaggacgaccagccacactgggactgagacacgg |
| | | cccagactcctacgggaggcagcagtggggaattttggacaatgggggcaaccctgatccagccatgccgcgtgcaggatg |
| | | aaggtcttcggattgtaaactgcttttgtcagggacgaaaagggatgcgataacaccgtattccgctgacggtacctgaagaata |
| | | agcaccggctaactacgtgccagcagccgcggtaatacgtagggtgcaagcgttaatcggaattactgggcgtaaagcgtgc |
| | | gcaggcggttctgtaagatagatgtgaaatccccgggctcaacctgggaattgcatatatgactgcaggacttgagtttgtcaga |
| | | ggagggtggaattccacgtgtagcagtgaaatgcgtagatatgtggaagaacaccgatggcgaaggcagccctctgggacat |
| | | gactgacgctcatgcacgaaagcgtggggagcaaacaggattagataccctggtagtccacgccctaaacgatgtctactagt |
| | | tgttggggacgatagtccttggtaacgcagctaacgcgtgaagtagaccgcctggggagtacggtcgcaagattaaaactcaa |
| | | aggaattgacggggacccgcacaagcggtggatgatgtggattaattcgatgcaacgcgaaaacccttccctagccttgacat |
| | | gccaggaaggtctgagagatcagaccgtgcccgcaagggaatctggacacaggtgctgcatggctgtcgtcagctcgtgtcg |
| | | tgagatgttgggttaagtcccgcaacgagcgcaacccttgtcattagttgctacgaaagggcactctaatgagactgccggtga |
| | | caaaccggaggaaggtggggatgacgtcaagtcctcatggcccttatggctagggcctcacacgtcatacaatggtcggaac |
| | | agagggaagcgaagccgcgaggtgaagccaatcccagaaaaccgatcgtagtccggattgcagtctgcaactcgactgcat |
| | | gaagtcggaatcgctagtaatcgcggatcagcatgccgcggtgaatacgacccgggtcttgtacacaccgcccgtcacacca |
| | | tgggagtggggttcaccagaagacgtttgcccaaccgaaaggaaggcggcgtccacggtgggcttcatgactggggtgaag |
| | | tcgtaacaaggtagccgtaccggaaggtgcggctggatcacctccttt |
| Sutterella_ | SEQ ID | taaagagtttgatcctggctcagattgaacgctggcggcatgctttacacatgcaagtcgaacggcagcgcggggagcttgct |
| wadsworthensis | NO: 246 | ccctggcggcgagtggcgcacgggtgagtaatacatcggaacgtgtcttctagtgggggataactgcccgaaagggcagct |
| | | aataccgcatgagacctgagggtgaaagcgggggatcgcaagacctcgcgctggaagagcggccgatgtccgattagcta |
| | | gttggtgaggtaaaggctcaccaaggcgacgatcggtagctggtctgagaggacgaccagccacactgggactgagacac |
| | | ggcccagactcctacgggaggcagcagtggggaattttggacaatgggggcaaccctgatccagccatgccgcgtgcagg |
| | | atgaaggtcttcggattgtaaactgcttttgtcagggacgaaaagggatgcgataacaccgtattccgctgacggtacctgaag |
| | | aataagcaccggctaactacgtgccagcagccgcggtaatacgtagggtgcaagcgttaatcggaattactgggcgtaaagc |
| | | gtgcgcaggcggttctgtaagatagatgtgaaatccccgggctcaacctgggaattgcatatatgactgcaggacttgagtttgt |
| | | cagaggagggtggaattccacgtgtagcagtgaaatgcgtagatatgtggaagaacaccgatggcgaaggcagccctctgg |
| | | gacatgactgacgctcatgcacgaaagcgtggggagcaaacaggattagataccctggtagtccacgccctaaacgatgtcta |
| | | ctagttgttggggacgatagtccttggtaacgcagctaacgcgtgaagtagaccgcctggggagtacggtcgcaagattaaaa |
| | | ctcaaaggaattgacggggacccgcacaagcggtggatgatgtggattaattcgatgcaacgcgaaaaaccttacctagcctt |
| | | gacatgccaggaaggcctgagagatcaggccgtgcccgcaagggaatctggacacaggtgctgcatggctgtcgtcagctc |
| | | gtgtcgtgagatgttgggttaagtcccgcaacgagcgcaacccttgtcattagttgctacgaaagggcactctaatgagactgc |
| | | cggtgacaaaccggaggaaggtggggatgacgtcaagtcctcatggcccttatggctagggcctcacacgtcatacaatggt |
| | | cggaacagagggaagcgaagccgcgaggtgaagccaatcccagaaaaccgatcgtagtccggattgcagtctgcaactcg |
| | | actgcatgaagtcggaatcgctagtaatcgcggatcagcatgccgcggtgaatacgttcccgggtcttgtacacaccgcccgt |
| | | cacaccatgggagtggggttcaccagaagacgtttgcccaaccgaaaggaaggcggcgtccacggtgggcttcatgactgg |
| | | ggtgaagtcgtaacaaggtagccgtaccggaaggtgcggctggatcacctccttt |
| Clostridium_ | SEQ ID | agagagtttgatcctggctcaggatgaacgctggcggcgtgcctaacacatgcaagtcgaacggagttatgcagaggaagttt |
| sp_KLE_1755 | NO: 247 | tcggatggaatcggcgtaacttagtggcggacgggtgagtaacgcgtgggaaacctgccctgtaccgggggataacacttag |
| | | aaataggtgctaataccgcataagcgcacagcttcacatgaggcagtgtgaaaaactccggtggtacaggatggtcccgcgtc |
| | | tgattagccagttggcagggtaacggcctaccaaagcgacgatcagtagccggcctgagagggtgaacggccacattggga |
| | | ctgagacacggcccaaactcctacgggaggcagcagtggggaatattgcacaatgggggaaaccctgatgcagcgacgcc |
| | | gcgtgagtgaagaagtatttcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgtagacggcat |
| | | gacaagccagatgtgaaaacccagggctcaaccctgggactgcatttggaactgccaggctggagtgcaggagaggtaagc |
| | | ggaattcctagtgtagcggtgaaatgcgtagatattaggaggaacaccagtggcgaaggcggcttactggactgtaactgacg |
| | | ttgaggctcgaaagcgtggggagcaaacaggattagataccctggtagtccacgcggtaaacgatgattgctaggtgtaggtg |
| | | ggtatggacccatcggtgccgcagctaacgcaataagcaatccacctggggagtacgttcgcaagaatgaaactcaaaggaa |
| | | ttgacggggacccgcacaagcggtggagcatgtggtttaattcgaagcaacgcgaagaaccttaccaagtcttgacatcccaa |
| | | tgacgtgcccgtaacggggcattctatcggagcattggagacaggtggtgcatggttgtcgtcagctcgtgtcgtgagatgttg |
| | | ggttaagtcccgcaacgagcgcaacccttatccttagtagccagcaggtaaagctgggcactctagggagactgccggggat |
| | | aacccggaggaaggcggggatgacgtcaaatcatcatgccccttatgatttgggctacacacgtgctacaatggcgtaaacaa |
| | | agggaagcgagacagtgatgttgagcaaatcccagaaataacgtctcagttcggattgtagtctgcaactcgactacatgaagc |
| | | tggaatcgctagtaatcgcgaatcagcatgtcgcggtgaatacgttcccgggtcttgtacacaccgcccgtcacaccatggga |
| | | gttggaaatgcccgaagcctgtgacctaaccgtaaggaaggagcagtcgaaggcaggtctaataactggggtgaagtcgtaa |
| | | caaggtagccgtatcggaaggtgcggctggatcacctccttt |
| Clostridium_ | SEQ ID | atgagagtttgatcctggctcaggatgaacgctggcggcgtgcctaacacatgcaagtcgaacgaagcgatttaacggaagttt |
| symbiosum | NO: 248 | tcggatggaagttgaattgactgagtggcggacgggtgagtaacgcgtgggtaacctgccttgtactgggggacaacagttag |
| | | aaatgactgctaataccgcataagcgcacagtattgcatgatacagtgtgaaaaactccggtggtacaagatggacccgcgtct |
| | | gattagctagttggtaaggtaacggcttaccaaggcgacgatcagtagccgacctgagagggtgaccggccacattgggact |
| | | gagacacggcccaaactcctacgggaggcagcagtggggaatattgcacaatgggcgaaagcctgatgcagcgacgccgc |
| | | gtgagtgaagaagtatttcggtatgtaaagctctatcagcagggaagaaaatgacggtacctgactaagaagccccggctaac |
| | | tacgtgccagcagccgcggtaatacgtagggggcaagcgttatccggatttactgggtgtaaagggagcgtagacggtaaag |
| | | caagtctgaagtgaaagcccgcggctcaactgcgggactgctttggaaactgtttaactggagtgtcggagaggtaagtggaa |
| | | ttcctagtgtagcggtgaaatgcgtagatattaggaggaacaccagtggcgaaggcgacttactggacgataactgacgttga |
| | | ggctcgaaagcgtggggagcaaacaggattagataccctggtagtccacgccgtaaacgatgaatactaggtgttggggagc |
| | | aaagctcttcggtgccgtcgcaaacgcagtaagtattccacctggggagtacgttcgcaagaatgaaactcaaaggaattgac |
| | | ggggacccgcacaagcggtggagcatgtggtttaattcgaagcaacgcgaagaaccttaccaggtcttgacatcgatccgac |
| | | gggggagtaacgtccccttcccttcggggcggagaagacaggtggtgcatggttgtcgtcagctcgtgtcgtgagatgttggg |
| | | ttaagtcccgcaacgagcgcaacccttattctaagtagccagcggttcggccgggaactcttgggagactgccagggataacc |
| | | tggaggaaggtggggatgacgtcaaatcatcatgccccttatgatctgggctacacacgtgctacaatggcgtaaacaaagag |
| | | aagcaagaccgcgaggtggagcaaatctcaaaaataacgtctcagttcggactgcaggctgcaactcgcctgcacgaagctg |
| | | gaatcgctagtaatcgcgaatcagaatgtcgcggtgaatacgttcccgggtcttgtacacaccgcccgtcacaccatgggagtc |
| | | agtaacgcccgaagtcagtgacccaaccgcaaggagggagctgccgaaggcgggaccgataactggggtgaagtcgtaa |
| | | caaggtagccgtatcggaaggtgcggctggatcacctccttt |
| Eubacterium_ | SEQ ID | gttctgtgactgagtggcggacgggtgagtaacgcgtggataacctgcctcacacagggggataacagttggaaacgactgc |
| ramulus | NO: 249 | taataccgcataagcgcacagcaccgcatggtgcagtgtgaaaaactccggtggtgtgagatggatccgcgtctgattagcttg |
| | | ttggcggggtaacggcccaccaaggcgacgatcagtagccggcctgagagggcgaccggccacattgggactgagacac |
| | | ggcccagactcctacgggaggcagcagtggggaatattgcacaatgggggaaaccctgatgcagcgacgccgcgtgagcg |
| | | atgaagtatttcggtatgtaaagctctatcagcagggaagataatgacggtacctgactaagaagctccggctaaatacgtgcc |
| | | agcagccgcggtaatacgtatggagcaagcgttatccggatttactgggtgtaaagggagcgtaggcggtcctgcaagtctga |
| | | tgtgaaaggccggggctcaaccccgggactgcattggaaactgtaggactagagtgtcggaggggtaagtggaattcctagt |
| | | gtagcggtgaaatgcgtagatattaggaggaacaccagtggcgaaggcggcttactggacgactactgacgctgaggctcga |
| | | aagcgtggggagcaaacaggattagataccctggtagtccacgccgtaaacgatgaatactaggtgttggcaggtaagacct |
| | | gtcggtgccgcagcaaacgcaataagtattccacctggggagtacgttcgcaagaatgaaactcaaaggaattgacggggac |
| | | ccgcacaagcggtggagcatgtggtttaattcgaagcaacgcgaagaaccttaccaagtcttgacatcccggtgacagaacat |
| | | gtaatgtgttttcccttcggggcaccggtgacaggtggtgcatggttgtcgtcagctcgtgtcgtgagatgttgggttaagtcccg |
| | | caacgagcgcaacccttatccccagtagccagcatttcggatgggcactctgtggagactgccagggacaacctggaggaag |
| | | gtggggatgacgtcaaatcatcatgccccttatgacttgggctacacacgtgctacaatggcgtaaacaaagggaagcgagac |
| | | cgcgaggtggagcaaatcccaaaaataacgtcccagttcggattgtagtctgcaactcgactacatgaagctggaatcgctagt |
| | | aatcgcagatcagaatgctgcggtgaatacgttcccgggtcttgtacacaccgcccgtcacaccatgggagtcggaaatgccc |
| | | gaagccggtggcctaaccgcaaggaaggagccgtcgaaggcaggtctgataactggggtgaagtcgtaacaaggtagccg |
| | | tatcggaaggtgcggctggatcacctccttt |
| Ruminococcus_ | SEQ ID | aacgagagtttgatcctggctcaggatgaacgctggcggcgtgcttaacacatgcaagtcgagcgaagcacttttgcggatttc |
| gnavus | NO: 250 | ttcggattgaagcaattgtgactgagcggcggacgggtgagtaacgcgtgggtaacctgcctcatacagggggataacagttg |
| | | gaaacggctgctaataccgcataagcgcacagtaccgcatggtaccgtgtgaaaaactccggtggtatgagatggacccgcg |
| | | tctgattagctagttggtggggtaacggcctaccaaggcgacgatcagtagccgacctgagagggtgaccggccacattggg |
| | | actgagacacggcccaaactcctacgggaggcagcagtggggaatattgcacaatgggggaaaccctgatgcagcgacgc |
| | | cgcgtgagcgatgaagtatttcggtatgtaaagctctatcagcagggaagaaaatgacggtacctgactaagaagccccggct |
| | | aactacgtgccagcagccgcggtaatacgtagggggcaagcgttatccggatttactgggtgtaaagggagcgtagacggca |
| | | tggcaagccagatgtgaaagcccggggctcaaccccgggactgcatttggaactgtcaggctagagtgtcggagaggaaag |
| | | cggaattcctagtgtagcggtgaaatgcgtagatattaggaggaacaccagtggcgaaggcggctttctggacgatgactgac |
| | | gttgaggctcgaaagcgtggggagcaaacaggattagataccctggtagtccacgccgtaaacgatgaatactaggtgtcgg |
| | | gtggcaaagccattcggtgccgcagcaaacgcaataagtattccacctggggagtacgttcgcaagaatgaaactcaaagga |
| | | attgacggggacccgcacaagcggtggagcatgtggtttaattcgaagcaacgcgaagaaccttacctggtcttgacatcccc |
| | | ctgaccgctctttaatcggagctttccttcgggacaggggagacaggtggtgcatggttgtcgtcagctcgtgtcgtgagatgtt |
| | | gggttaagtcccgcaacgagcgcaacccctatctttagtagccagcatttcggatgggcactctagagagactgccagggata |
| | | acctggaggaaggtggggatgacgtcaaatcatcatgccccttatgaccagggctacacacgtgctacaatggcgtaaacaaa |
| | | gggaagcgagcccgcgagggggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gtcagtaacgcccgaagtcagtgacccaaccgcaaggagggagctgccgaaggtgggaccgataactggggtgaagtcgt |
| | | aacaaggtagccgtatcggaaggtgcggctggatcacctccttt |
| Ruminococcus_ | SEQ ID | aacgagagtttgatcctggctcaggatgaacgctggcggcgtgcttaacacatgcaagtcgagcgaagcaccttgacggattt |
| gnavus | NO: 251 | cttcggattgaagccttggtgactgagcggcggacgggtgagtaacgcgtgggtaacctgcctcatacagggggataacagtt |
| | | ggaaacggctgctaataccgcataagcgcacagtaccgcatggtacggtgtgaaaaactccggtggtatgagatggacccgc |
| | | gtctgattaggtagttggtggggtaacggcctaccaagccgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgatgaagtatttcggtatgtaaagctctatcagcagggaagaaaatgacggtacctgactaagaagccccggc |
| | | taactacgtgccagcagccgcggtaatacgtagggggcaagcgttatccggatttactgggtgtaaagggagcgtagacggc |
| | | atggcaagccagatgtgaaagcccggggctcaaccccgggactgcatttggaactgtcaggctagagtgtcggagaggaaa |
| | | gcggaattcctagtgtagcggtgaaatgcgtagatattaggaggaacaccagtggcgaaggcggctttctggacgatgactga |
| | | cgttgaggctcgaaagcgtggggagcaaacaggattagataccctggtagtccacgccgtaaacgatgaatactaggtgtcg |
| | | ggtggcaaagccattcggtgccgcagcaaacgcaataagtattccacctggggagtacgttcgcaagaatgaaactcaaagg |
| | | aattgacggggacccgcacaagcggtggagcatgtggtttaattcgaagcaacgcgaagaaccttacctggtcttgacatccc |
| | | tctgaccgctctttaatcggagctttccttcgggacagaggagacaggtggtgcatggttgtcgtcagctcgtgtcgtgagatgtt |
| | | gggttaagtcccgcaacgagcgcaacccctatctttagtagccagcattttggatgggcactctagagagactgccagggata |
| | | acctggaggaaggtggggatgacgtcaaatcatcatgccccttatgatcagggctacacacgtgctacaatggcgtaaacaaa |
| | | gggaagcgagcccgcgagggggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gtcagtaacgcccgaagtcagtgacccaaccgcaaggagggagctgccgaaggtgggaccgataactggggtgaagtcgt |
| | | aacaaggtagccgtatcggaaggtgcggctggatcacctccttt |
| Ruminococcus_ | SEQ ID | aacgagagtttgatcctggctcaggatgaacgctggcggcgtgcttaacacatgcaagtcgagcgaagcaccttgacggattt |
| gnavus | NO: 252 | cttcggattgaagccttggtgactgagcggcggacgggtgagtaacgcgtgggtaacctgcctcatacagggggataacagtt |
| | | ggaaacggctgctaataccgcataagcgcacagtaccgcatggtacggtgtgaaaaactccggtggtatgagatggacccgc |
| | | gtctgattaggtagttggtggggtaacggcctaccaagccgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgatgaagtatttcggtatgtaaagctctatcagcagggaagaaaatgacggtacctgactaagaagccccggc |
| | | taactacgtgccagcagccgcggtaatacgtagggggcaagcgttatccggatttactgggtgtaaagggagcgtagacggc |
| | | atggcaagccagatgtgaaagcccggggctcaaccccgggactgcatttggaactgtcaggctagagtgtcggagaggaaa |
| | | gcggaattcctagtgtagcggtgaaatgcgtagatattaggaggaacaccagtggcgaaggcggctttctggacgatgactga |
| | | cgttgaggctcgaaagcgtggggagcaaacaggattagataccctggtagtccacgccgtaaacgatgaatactaggtgtcg |
| | | ggtggcaaagccattcggtgccgcagcaaacgcaataagtattccacctggggagtacgttcgcaagaatgaaactcaaagg |
| | | aattgacggggacccgcacaagcggtggagcatgtggtttaattcgaagcaacgcgaagaaccttacctggtcttgacatccc |
| | | cctgaccgctctttaatcggagctttccttcgggacagaggagacaggtggtgcatggttgtcgtcagctcgtgtcgtgagatgt |
| | | tgggttaagtcccgcaacgagcgcaacccctatctttagtagccagcattttggatgggcactctagagagactgccagggata |
| | | acctggaggaaggtggggatgacgtcaaatcatcatgccccttatgatcagggctacacacgtgctacaatggcgtaaacaaa |
| | | gggaagcgagcccgcgagggggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gtcagtaacgcccgaagtcagtgacccaaccgcaaggagggagctgccgaaggtgggaccgataactggggtgaagtcgt |
| | | aacaaggtagccgtatcggaaggtgcggctggatcacctccttt |
| Ruminococcus_ | SEQ ID | ncgacgatcagtagccgacctgagagggtgaccggccacattgggactgagacacggcccaaactcctacgggaggcagc |
| gnavus | NO: 253 | agtggggaatattgcacaatgggggaaaccctgatgcagcgacgccgcgtgagcgatgaagtatttcggtatgtaaagctcta |
| | | tcagcagggaagaaaatgacggtacctgactaagaagccccggctaactacgtgccagcagccgcggtaatacgtagggg |
| | | gcaagcgttatccggatttactgggtgtaaagggagcgtagacggcatggcaagccagatgtgaaagcccggggctcaacc |
| | | ccgggactgcatttggaactgtcaggctagagtgtcggagaggaaagcggaattcctagtgtagcggtgaaatgcgtagatat |
| | | taggaggaacaccagtggcgaaggcggctttctggacgatgactgacgttgaggctcgaaagcgtggggagcaaacaggat |
| | | tagataccctggtagtccacgccgtaaacgatgaatactaggtgtcgggtggcaaagccattcggtgccgcagcaaacgcaat |
| | | aagtattccacctggggagtacgttcgcaagaatgaaactcaaaggaattgacggggacccgcacaagcggtggagcatgtg |
| | | gtttaattcgaagcaacgcgaagaaccttacctggtcttgacatccctctgaccgctctttaatcggaactttccttcgggacaga |
| | | ggagacaggtggtgcatggttgtcgtcagctcgtgtcgtgagatgttgggttaagtcccgcaacgagcgcaacccctatcttta |
| | | gtagccagcattttggatgggcactctagagagactgccagggataacctggaggaaggtggggatgacgtcaaatcatcat |
| | | gccccttatgaccagggctacacacgtgctacaatggcgtaaacaaagggaagcgagcccgcgagggggagcaaatccca |
| | | aaaataacgtctcagttcggattgtagtctgcaactcgactacatgaagctggaatcgctagtaatcgcgaatcagaatgtcgcg |
| | | gtgaatacgttcccgggtcttgtacacaccgcccgtcacaccatgggagtcagtaacgcccgaagtcagtgacccaaccgca |
| | | aggagggagctgccgaaggtgggaccgataactggggtgaagtcgtaacaaggtagccgtatcggaaggtgcggctggat |
| | | cacctccttt |
| Ruminococcus_ | SEQ ID | aacgagagtttgatcctggctcaggatgaacgctggcggcgtgcttaacacatgcaagtcgagcgaagcaccttgacggattt |
| gnavus | NO: 254 | cttcggattgaagccttggtgactgagcggcggacgggtgagtaacgcgtgggtaacctgcctcatacagggggataacagtt |
| | | ggaaacggctgctaataccgcataagcgcacagtaccgcatggtacggtgtgaaaaactccggtggtatgagatggacccgc |
| | | gtctgattaggtagttggtggggtaacggcctaccaagccgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgatgaagtatttcggtatgtaaagctctatcagcagggaagaaaatgacggtacctgactaagaagccccggc |
| | | taactacgtgccagcagccgcggtaatacgtagggggcaagcgttatccggatttactgggtgtaaagggagcgtagacggc |
| | | atggcaagccagatgtgaaagcccggggctcaaccccgggactgcatttggaactgtcaggctagagtgtcggagaggaaa |
| | | gcggaattcctagtgtagcggtgaaatgcgtagatattaggaggaacaccagtggcgaaggcggctttctggacgatgactga |
| | | cgttgaggctcgaaagcgtggggagcaaacaggattagataccctggtagtccacgccgtaaacgatgaatactaggtgtcg |
| | | ggtggcaaagccattcggtgccgcagcaaacgcaataagtattccacctggggagtacgttcgcaagaatgaaactcaaagg |
| | | aattgacggggacccgcacaagcggtggagcatgtggtttaattcgaagcaacgcgaagaaccttacctggtcttgacatccc |
| | | tctgaccgctctttaatcggagctttccttcgggacagaggagacaggtggtgcatggttgtcgtcagctcgtgtcgtgagatgtt |
| | | gggttaagtcccgcaacgagcgcaacccctatctttagtagccagcatttcggatgggcactctagagagactgccagggata |
| | | acctggaggaaggtggggatgacgtcaaatcatcatgccccttatgaccagggctacacacgtgctacaatggcgtaaacaaa |
| | | gggaagcgagcccgcgagggggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gtcagtaacgcccgaagtcagtgacccaaccgcaaggagggagctgccgaaggtgggaccgataactggggtgaagtcgt |
| | | aacaaggtagccgtatcggaaggtgcggctggatcacctccttt |
| Blautia_wexlerae | SEQ ID | tcagagagtttgatcctggctcaggatgaacgctggcggcgtgcttaacacatgcaagtcgaacgggaaatatttcattgagac |
| | NO: 255 | ttcggtggatttgatctatttctagtggcggacgggtgagtaacgcgtgggtaacctgccttatacagggggataacagtcagaa |
| | | atggctgctaataccgcataagcgcacagagctgcatggctcagtgtgaaaaactccggtggtataagatggacccgcgttgg |
| | | attagctagttggtggggtaacggcccaccaaggcgacgatccatagccggcctgagagggtgaacggccacattgggact |
| | | gagacacggcccagactcctacgggaggcagcagtggggaatattgcacaatgggggaaaccctgatgcagcgacgccg |
| | | cgtgaaggaagaagtatctcggtatgtaaacttctatcagcagggaagatagtgacggtacctgactaagaagccccggctaa |
| | | ctacgtgccagcagccgcggtaatacgtagggggcaagcgttatccggatttactgggtgtaaagggagcgtagacggtgtg |
| | | gcaagtctgatgtgaaaggcatgggctcaacctgtggactgcattggaaactgtcatacttgagtgccggaggggtaagcgga |
| | | attcctagtgtagcggtgaaatgcgtagatattaggaggaacaccagtggcgaaggcggcttactggacggtaactgacgttg |
| | | aggctcgaaagcgtggggagcaaacaggattagataccctggtagtccacgccgtaaacgatgaatactaggtgtcgggtgg |
| | | cagagccattcggtgccgtcgcaaacgcagtaagtattccacctggggagtacgttcgcaagaatgaaactcaaaggaattga |
| | | cggggacccgcacaagcggtggagcatgtggtttaattcgaagcaacgcgaagaaccttaccaagtcttgacatccgcctga |
| | | ccgatccttaaccggatctttccttcgggacaggcgagacaggtggtgcatggttgtcgtcagctcgtgtcgtgagatgttgggt |
| | | taagtcccgcaacgagcgcaacccctatcctcagtagccagcatttaaggtgggcactctggggagactgccagggataacct |
| | | ggaggaaggcggggatgacgtcaaatcatcatgccccttatgatttgggctacacacgtgctacaatggcgtaaacaaaggg |
| | | aagcgagattgtgagatggagcaaatcccaaaaataacgtcccagttcggactgtagtctgcaacccgactacacgaagctgg |
| | | aatcgctagtaatcgcggatcagaatgccgcggtgaatacgttcccggg |
| Gemmiger_formicilis | SEQ ID | aaaggatttattcgcttcaggatggactcgcgtccaattagctagttggtgaggtaacggcccaccaaggcgacgattggtagc |
| | NO: 256 | cggactgagaggttgaacggccacattgggactgagacacggcccagactcctacgggaggcagcagtgggggatattgc |
| | | acaatgggggaaaccctgatgcagcgacgccgcgtggaggaagaaggttttcggattgtaaactcctgtcgttagggacgat |
| | | aatgacggtacctaacaagaaagcaccggctaactacgtgccagcagccgcggtaaaacgtagggtgcaagcgttgtccgg |
| | | aattactgggtgtaaagggagcgcaggcggaccggcaagttggaagtgaaatccatgggctcaacccgtgaattgctttcaaa |
| | | actgctggccttgagtagtgcagaggtaggtggaattcccggtgtagcggtggaatgcgtagatatcgggaggaacaccagt |
| | | ggcgaaggcgacctactgggcaccaactgacgctgaggctcgaaagcatgggtagcaaacaggattagataccctggtagt |
| | | ccatgccgtaaacgatgattactaggtgttggaggattgaccccttcagtgccgcagttaacacaataagtaatccacctgggg |
| | | agtacgaccgcaaggttgaaactcaaaggaattgacgggggcccgcacaagcagtggagtatgtggtttaattcgaagcaac |
| | | gcgaagaaccttaccaggtcttgacatccgatgcatagtgcagagatgcatgaagtccttcgggacatcgagacaggtggtgc |
| | | atggttgtcgtcagctcgtgtcgtgagatgttgggttaagtcccgcaacgagcgcaacccttattgccagttactacgcaagagg |
| | | actctggcgagactgccgttgacaaaacggaggaaggtggggatgacgtcaaatcatcatgccctttatgacctgggctacac |
| | | acgtactacaatggcgtttaacaaagagaagcaagaccgcgaggtggagcaaaactcagaaacaacgtctcagttcagattg |
| | | caggctgcaactcgcctgcatgaagtcggaattgctagtaatcgcggatcagcatgccgcggtgaatacgttcccgggccttgt |
| | | acacaccgcccgtcacaccatgagagccggggggacccgaagtcggtagtctaaccgcaaggaggacgccgccgaaggt |
| | | aaaactggtgattggggtgaagtcgtaacaaggtagccgtatcggaaggtgcggctggatcacctccttt |
| Eisenbergiella_ | SEQ ID | agagagtttgatcctggctcaggatgaacgctggcggcgtgcctaacacatgcaagtcgaacggagttatgcagaggaagttt |
| massiliensis | NO: 257 | tcggatggaatcggcgtaacttagtggcggacgggtgagtaacgcgtgggaaacctgccctgtaccgggggataacacttag |
| | | aaataggtgctaataccgcataagcgcacagcttcacatgaggcagtgtgaaaaactccggtggtacaggatggtcccgcgtc |
| | | tgattagccagttggcagggtaacggcctaccaaagcgacgatcagtagccggcctgagagggtgaacggccacattggga |
| | | ctgagacacggcccaaactcctacgggaggcagcagtggggaatattgcacaatgggggaaaccctgatgcagcgacgcc |
| | | gcgtgagtgaagaagtatttcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgtagacggcat |
| | | gacaagccagatgtgaaaacccagggctcaaccctgggactgcatttggaactgccaggctggagtgcaggagaggtaagc |
| | | ggaattcctagtgtagcggtgaaatgcgtagatattaggaggaacaccagtggcgaaggcggcttactggactgtaactgacg |
| | | ttgaggctcgaaagcgtggggagcaaacaggattagataccctggtagtccacgcggtaaacgatgattgctaggtgtaggtg |
| | | ggtatggacccatcggtgccgcagctaacgcaataagcaatccacctggggagtacgttcgcaagaatgaaactcaaaggaa |
| | | ttgacggggacccgcacaagcggtggagcatgtggtttaattcgaagcaacgcgaagaaccttaccaagtcttgacatcccaa |
| | | tgacgtgtccgtaacggggcattctcttcggagcattggagacaggtggtgcatggttgtcgtcagctcgtgtcgtgagatgttg |
| | | ggttaagtcccgcaacgagcgcaacccttatccttagtagccagcaggtaaagctgggcactctagggagactgccggggat |
| | | aacccggaggaaggcggggatgacgtcaaatcatcatgccccttatgatttgggctacacacgtgctacaatggcgtaaacaa |
| | | agggaagcgagacagtgatgttgagcaaatcccagaaataacgtctcagttcggattgtagtctgcaactcgactacatgaagc |
| | | tggaatcgctagtaatcgcgaatcagcatgtcgcggtgaatacgttcccgggtcttgtacacaccgcccgtcacaccatggga |
| | | gttggaaatgcccgaagcctgtgacctaaccgcaagggaggagcagtcgaaggcaggtctaataactggggtgaagtcgta |
| | | acaaggtagccgtatcggaaggtgcggctggatg |
| Ruminococcus_ | SEQ ID | ctaaagagtttgatcctggctcaggacgaacgctggcggcgcgcctaacacatgcaagtcgaacggagaaatgctgagcttg |
| sp. | NO: 258 | ctttgcatttcttagtggcggacgggtgagtaacacgtgagcaacctgcctttgtgaggggaataacgtctggaaacggacgct |
| | | aataccgcataacgtcaaggaaccgcatggttttttgaccaaagattttatcgcacaaagatgggctcgcggctgattagctagtt |
| | | ggcggggtaacggcccaccaaggcgacgatcagtagccggactgagaggttgatcggccacattgggactgagacacggc |
| | | ccagactcctacgggaggcagcagtgggggatattgcacaatgggggaaaccctgatgcagcgacgccgcgtgagggaa |
| | | gacggttttcggattgtaaacctctgtcttcagggacgaaatcaatgacggtacctgaggaggaagccacggctaactacgtgc |
| | | cagcagccgcggtaatacgtaggtggcaagcgttgtccggaattactgggtgtaaagggagcgtaggcgggaatgcaagttg |
| | | aatgtttaaactatcggctcaactgataatcgcgttcaaaactgcatttcttgagtggagtagaggcaggcggaattcctagtgta |
| | | gcggtgaaatgcgtagatattaggaggaacaccagtggcgaaggcggcctgctgggctctaactgacgctgaggctcgaaa |
| | | gcgtgggtagcaaacaggattagataccctggtagtccacgccgtaaacgatgattactaggtgtggggggactgaccccttc |
| | | cgtgccggagttaacacaataagtaatccacctggggagtacggtcgcaagactgaaactcaaaggaattgacgggggccc |
| | | gcacaagcagtggagtatgtggtttaattcgaagcaacgcgaagaaccttaccaggtcttgacatcgtgcgcataccgtagag |
| | | atacgggaagtccttcgggacgcatagacaggtggtgcatggttgtcgtcagctcgtgtcgtgagatgttgggttaagtcccgc |
| | | aacgagcgcaacccttattattagttgctacgcaagagcactctaatgagactgccgttgacaaaacggaggaaggtggggat |
| | | gacgtcaaatcatcatgccccttatgacctgggctacacacgtactacaatggcacttaacagagggaagcaagaccgcgag |
| | | gtggagcaaacccccaaaaagtgtctcagttcggattgcaggctgcaacccgcctgtatgaagtcggaattgctagtaatcgc |
| | | ggatcagcatgccgcggtgaatacgttcccgggccttgtacacaccgcccgtcacaccatgagagccggtaacacccgaagt |
| | | cagtagcctaaccgcaaggagggcgctgccgaaggtgggattggtgattagggtgaagtcgtaacaaggtagccgtatcgg |
| | | aaggtgcggctggatcacctccttt |
| Roseburia_ | SEQ ID | atgagagtttgatcctggctcaggatgaacgctggcggcgtgcttaacacatgcaagtcgaacgaagcactctatttgattttctt |
| faecis | NO: 259 | cggaaatgaagattttgtgactgagtggcggacgggtgagtaacgcgtgggtaacctgcctcatacagggggataacagttg |
| | | gaaacgactgctaataccgcataagcgcacaggattgcatgatccggtgtgaaaaactccggtggtatgggatggacccgcg |
| | | tctgattagccagttggcagggtaacggcctaccaaagcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgaagaagtatttcggtatgtaaagctctatcagcaggaaagaaaaatgacggtacctgactaagaagcaccg |
| | | gctaaatacgtgccagcagccgcggtaatacgtatggtgcaagcgttatccggatttactgggtgtaaagggagcgcaggcg |
| | | gtgcggcaagtctgatgtgaaagcccggggctcaaccccggtactgcattggaaactgtcgtactagagtgtcggaggggta |
| | | agtggaattcctagtgtagcggtgaaatgcgtagatattaggaggaacaccagtggcgaaggcggcttactggacgataactg |
| | | acgctgaggctcgaaagcgtggggagcaaacaggattagataccctggtagtccacgccgtaaacgatgaatactaggtgtc |
| | | ggggagcattgctcttcggtgccgcagcaaacgcaataagtattccacctggggagtacgttcgcaagaatgaaactcaaagg |
| | | aattgacggggacccgcacaagcggtggagcatgtggtttaattcgaagcaacgcgaagaaccttaccaagtcttgacatccc |
| | | gatgacagagtatgtaatgtactttctcttcggagcatcggtgacaggtggtgcatggttgtcgtcagctcgtgtcgtgagatgtt |
| | | gggttaagtcccgcaacgagcgcaacccctgttcttagtagccgcggttcggccgggcactctagggagactgccagggat |
| | | aacctggaggaaggcggggatgacgtcaaatcatcatgccccttatgacttgggctacacacgtgctacaatggcgtaaacaa |
| | | agggaagcggagccgtgaggccgagcaaatctcaaaaataacgtctcagttcggactgtagtctgcaacccgactacacgaa |
| | | gctggaatcgctagtaatcgcagatcagaatgctgcggtgaatacgttcccgggtcttgtacacaccgcccgtcacaccatgg |
| | | gagttggaaatgcccgaagtcagtgacccaaccgcaaggagggagctgccgaaggcaggttcgataactggggtgaagtc |
| | | gtaacaaggtagccgtatcggaaggtgcggctggatcacctccttt |
| Butyrivibrio_ | SEQ ID | ttgagagtttgatcctggctcaggatgaacgctggcggcgtgcttaacacatgcaagtcgaacggacggattggagcttgctcc |
| sp. | NO: 260 | atgaagttagtggcggacgggtgagtaacgcgtgggtaacctgccctatgcagggggataacgtttggaaacgaacgctaat |
| | | accgcataacctagttggttcgcatgaaccgactagcaaagattttatcggcataggatggacccgcgttggattagctagttgg |
| | | tgagataacagcccaccaaggcaacgatccatagccggcctgagagggtgaacggccacactgggactgagacacggccc |
| | | agactcctacgggaggcagcagtggggaatattgcacaatgggcgcaagcctgatgcagcgacgccgcgtgaaggatgaa |
| | | ggtcttcggatcgtaaacttctatcagcagggaagaaaccatgacggtacctgactaagaagccccggctaactacgtgccag |
| | | cagccgcggtaatacgtagggggcaagcgttatccggaattactgggtgtaaagggtgcgtaggcggcgatttaagtcagat |
| | | gtgaaaactcagggctcaaccttgagactgcatctgaaactgagttgctagagtgcaggagaggaaagcggaattccgagtgt |
| | | agcggtgaaatgcgtagagattcggaggaacaccagtagcgaaggcggctttctggactgtaactgacgctgaggcacgaa |
| | | agcgtggggagcgaacaggattagataccctggtagtccacgccgtaaacgatgaatactaggtgtcggggcttacgggtct |
| | | cggtgccgcagctaacgcattaagtattccacctgggaagtacgaccgcaaggttgaaactcaaaggaattgacggggaccc |
| | | gcacaagcggtggagcatgtggtttaattcgaagcaacgcgaagaaccttacctaaacttgacatccctttgaccggtccttaat |
| | | cggatctttactagcttgctagtacaaaggtgacaggtggtgcatggttgtcgtcagctcgtgtcgtgagatgttgggttaagtcc |
| | | cgcaacgagcgcaacccctatctttagtagccatcattcagttgggcactctagagagactgccagggataacctggaggaag |
| | | gtggggatgacgtcaaatcatcatgccccttatgtttagggctacacacgtgctacaatgggtgctacaaagggaagcaaatcc |
| | | gcgaggccgagcaaatctcaaaaaagcactcccagttcggattgtagtctgcaactcgactacatgaagttggaatcgctagta |
| | | atcgcgaatcagaatgtcgcggtgaatacgttcccgggtcttgtacacaccgcccgtcacaccatgggagttgggggggccc |
| | | aacgccagtgacctaacccgcaagggaaggagctgtctaaggcaaaaccaatgactggggtgaagtcgtaacaaggtagcc |
| | | gtatcggaaggtgcggctggatcacctccttt |
| Blautia_sp_ | SEQ ID | acagagagtttgatcctggctcaggatgaacgctggcggcgtgcttaacacatgcaagtcgaacggggattattttgacagag |
| CAG_257 | NO: 261 | acttcggttgaagtcgttataatcctagtggcggacgggtgagtaacgcgtgggtaacctgcctcacactgggggataacagtc |
| | | agaaatgactgctaataccgcataagcgcacagagctgcatggctcagtgtgaaaaactccggtggtgtgagatggacccgc |
| | | gttggattagccagttggcagggtaacggcctaccaaagcgacgatccatagccggcctgagagggtggacggccacattg |
| | | ggactgagacacggcccagactcctacgggaggcagcagtggggaatattgcacaatgggggaaaccctgatgcagcgac |
| | | gccgcgtgaaggaagaagtatctcggtatgtaaacttctatcagcagggaagaaaatgacggtacctgactaagaagccccg |
| | | gctaactacgtgccagcagccgcggtaatacgtagggggcaagcgttatccggatttactgggtgtaaagggagcgtagacg |
| | | gagcagcaagtctgatgtgaaaggcgggggctcaacccccggactgcattggaaactgttgatcttgagtaccggagaggta |
| | | agcggaattcctagtgtagcggtgaaatgcgtagatattaggaggaacaccagtggcgaaggcggcttactggacggtaact |
| | | gacgttgaggctcgaaagcgtggggagcaaacaggattagataccctggtagtccacgccgtaaacgatgaatactaggtgt |
| | | cgggtggcagagccattcggtgccgcagcaaacgcagtaagtattccacctggggagtacgttcgcaagaatgaaactcaaa |
| | | ggaattgacggggacccgcacaagcggtggagcatgtggtttaattcgaagcaacgcgaagaaccttaccaagtcttgacatc |
| | | cctctgaccggtgagtaacgtcacctttccttcgggacagaggagacaggtggtgcatggttgtcgtcagctcgtgtcgtgaga |
| | | tgttgggttaagtcccgcaacgagcgcaacccctatccttagtagccagcggtttggccgggcactctgaggagactgccagg |
| | | gataacctggaggaaggcggggatgacgtcaaatcatcatgccccttatgatttgggctacacacgtgctacaatggcgtaaa |
| | | caaagggaagcgagagtgtgagcttaagcaaatcccaaaaataacgtcccagttcggactgcagtctgcaactcgactgcac |
| | | gaagctggaatcgctagtaatcgcggatcagaatgccgcggtgaatacgttcccgggtcttgtacacaccgcccgtcacacca |
| | | tgggagtcagtaacgcccgaagtcagtgaccgaaccgaaaggacggagctgccgaaggcgggacggatgactggggtga |
| | | agtcgtaacaaggtagccgtatcggaaggtgcggctggatcacctccttt |
| Eubacterium_ | SEQ ID | gaacggccacattgggactgagacacggcccagactcctacgggaggcagcagtggggaatattgcgcaatgggggaaac |
| sp_3_1_31 | NO: 262 | cctgacgcagcaacgccgcgtgattgaagaaggccttcgggttgtaaagatctttaattggggacgaaaaatgacggtaccca |
| | | aagaataagctccggctaactacgtgccagcagccgcggtaatacgtagggagcaagcgttatccggatttactgggtgtaaa |
| | | gggcgagtaggcgggctggcaagagggagtgaaatcccggggcttaaccccggaactgctttcaaaactgctggtcttgagt |
| | | gatggagaggcaggcggaattccgtgtgtagcggtgaaatgcgtagatatacggaggaacaccagtggcgaaggcggcct |
| | | gctggacattaactgacgctgaggagcgaaagcgtggggagcaaacaggattagataccctggtagtccacgccgtaaacg |
| | | atggatactaggtgtgggaggtattgaccccttccgtgccggagttaacacaataagtatcccacctggggagtacggccgca |
| | | aggttgaaactcaaaggaattgacgggggcccgcacaagcagtggagtatgtggtttaattcgaagcaacgcgaagaacctt |
| | | accaggtcttgacatccctctgaccgccctagagatagggtttcccttcggggcagaggtgacaggtggtgcatggttgtcgtc |
| | | agctcgtgtcgtgagatgttgggttaagtcccgcaacgagcgcaacccttacggttagttgatacgcaagatcactctagccgg |
| | | actgccgttgacaaaacggaggaaggtggggacgacgtcaaatcatcatgccccttatgacctgggctacacacgtactacaa |
| | | tggcagtcatacagagggaagcaaaacagtgatgtggagcaaatccctaaaagctgtcccagttcagattgcaggctgcaact |
| | | cgcctgcatgaagtcggaattgctagtaatcgcggatcagcatgccgcggtgaatacgttcccgggccttgtacacaccgccc |
| | | gtcacaccatgagagccggtaatacccgaagtccgtagcctaaccgcaaggagggcgcggccgaaggtaggactggtaatt |
| | | agggtgaagtcgtaacaaggtagccgtatcggaaggtgcggctggatcacctccttt |
| Gemmiger_ | SEQ ID | aaaaggatttattcgctttaggatggactcgcgtccaattagctagttggtgaggtaacggcccaccaaggcgacgattggtag |
| formicilis | NO: 263 | ccggactgagaggttgaacggccacattgggactgagacacggcccagactcctacgggaggcagcagtgggggatattg |
| | | cacaatgggggaaaccctgatgcagcgacgccgcgtggaggaagaaggttttcggattgtaaactcctgtcgttagggacga |
| | | taatgacggtacctaacaagaaagcaccggctaactacgtgccagcagccgcggtaaaacgtagggtgcaagcgttgtccgg |
| | | aattactgggtgtaaagggagcgcaggcgggaagacaagttggaagtgaaaaccatgggctcaacccatgaattgctttcaa |
| | | aactgtttttcttgagtagtgcagaggtagatggaattcccggtgtagcggtggaatgcgtagatatcgggaggaacaccagtg |
| | | gcgaaggcggtctactgggcaccaactgacgctgaggctcgaaagcatgggtagcaaacaggattagataccctggtagtcc |
| | | atgccgtaaacgatgattactaggtgttgggggattgaccccctcagtgccgcagttaacacaataagtaatccacctggggag |
| | | tacgaccgcaaggttgaaactcaaaggaattgacgggggcccgcacaagcagtggagtatgtggtttaattcgaagcaacgc |
| | | gaagaaccttaccaggtcttgacatccgatgcatagcacagagatgtgtgaaatctttcgggacatcgagacaggtggtgcat |
| | | ggttgtcgtcagctcgtgtcgtgagatgttgggttaagtcccgcaacgagcgcaacccttattgccagttactacgttaagagga |
| | | ctctggcgagactgccgttgacaaaacggaggaaggtggggatgacgtcaaatcatcatgccctttatgacctgggctacaca |
| | | cgtactacaatggcgttaaacaaagagaagcaagaccgcgaggtggagcaaaactcaaaaacaacgtctcagttcagattgc |
| | | aggctgcaactcgcctgcatgaagtcggaattgctagtaatcgcggatcagcatgccgcggtgaatacgttcccgggccttgta |
| | | cacaccgcccgtcacaccatgagagccggggggacccgaagtcgatagtctaaccgcaaggaggacgtcgccgaaggtaa |
| | | aactggtgattggggtgaagtcgtaacaaggtagccgtatcggaaggtgcggctggatcacctccttt |
| Intestinimonas_ | SEQ ID | ggctcaccaaggcgacgatcagtagccggactgagaggttggccggccacattgggactgagacacggcccagactccta |
| butyriciproducens | NO: 264 | cgggaggcagcagtggggaatattgggcaatgggcgaaagcctgacccagcaacgccgcgtgaaggaagaaggccctcg |
| | | ggttgtaaacttcttttgtcagggacgaagcaagtgacggtacctgacgaataagccacggctaactacgtgccagcagccgc |
| | | ggtaatacgtaggtggcaagcgttatccggatttactgggtgtaaagggcgtgtaggcgggagtgcaagtcagatgtgaaaac |
| | | tatgggctcaacccatagcctgcatttgaaactgtacttcttgagtgatggagaggcaggcggaattccctgtgtagcggtgaaa |
| | | tgcgtagatatagggaggaacaccagtggcgaaggcggcctgctggacattaactgacgctgaggcgcgaaagcgtgggg |
| | | agcaaacaggattagataccctggtagtccacgccgtaaacgatggatactaggtgtggggggtctgaccccctccgtgccg |
| | | cagttaacacaataagtatcccacctggggagtacgatcgcaaggttgaaactcaaaggaattgacgggggcccgcacaagc |
| | | ggtggagtatgtggtttaattcgaagcaacgcgaagaaccttaccaggacttgacatcctactaacgaagcagagatgcataag |
| | | gtgcccttcggggaaagtagagacaggtggtgcatgttgtcgtcagctcgtgtcgtgagatgttgggttaagtcccgcaacga |
| | | gcgcaacccttattgttagttgctacgcaagagcactctagcgagactgccgttgacaaaacggaggaaggtggggacgacg |
| | | tcaaatcatcatgccccttatgtcctgggccacacacgtactacaatggcggtcaacagagggaagcaaagccgcgaggtgg |
| | | agcaaatccctaaaagccgtcccagttcggattgcaggctgaaactcgcctgtatgaagtcggaatcgctagtaatcgcggatc |
| | | agcatgccgcggtgaatacgttcccgggccttgtacacaccgcccgtcacaccatgagagtcgggaacacccgaagtccgta |
| | | gcctaacagcaatgggggcgcggccgaaggtgggttcgataattggggtgaagtcgtaacaaggtagccgtatcggaaggt |
| | | gcggctggatcacctccttt |
| Oscillospiraceae_ | SEQ ID | tatagagagtttgatcctggctcaggacgaacgctggcggcgtgcttaacacatgcaagtcgaacggagcacccttgactgag |
| bacterium_ | NO: 265 | gtttcggccaaatgataggaatgcttagtggcggactggtgagtaacgcgtgaggaacctaccttccagagggggacaacag |
| VE202_ | | ttggaaacgactgctaataccgcatgacgcatgaccggggcatcccgggcatgtcaaagattttatcgctggaagatggcctc |
| 24 | | gcgtctgattagctagatggtggggtaacggcccaccatggcgacgatcagtagccggactgagaggttgaccggccacatt |
| | | gggactgagatacggcccagactcctacgggaggcagcagtggggaatattgggcaatggacgcaagtctgacccagcaa |
| | | cgccgcgtgaaggaagaaggctttcgggttgtaaacttcttttgtcagggaagagtagaagacggtacctgacgaataagcca |
| | | cggctaactacgtgccagcagccgcggtaatacgtaggtggcaagcgttgtccggatttactgggtgtaaagggcgtgcagc |
| | | cgggccggcaagtcagatgtgaaatctggaggcttaacctccaaactgcatttgaaactgtaggtcttgagtaccggagaggtt |
| | | atcggaattccttgtgtagcggtgaaatgcgtagatataaggaagaacaccagtggcgaaggcggataactggacggcaact |
| | | gacggtgaggcgcgaaagcgtggggagcaaacaggattagataccctggtagtccacgctgtaaacgatggatactaggtgt |
| | | gcggggactgaccccctgcgtgccgcagttaacacaataagtatcccacctggggagtacgatcgcaaggttgaaactcaaa |
| | | ggaattgacgggggcccgcacaagcggtggattatgtggtttaattcgaagcaacgcgaagaaccttaccagggcttgacatc |
| | | ctactaacgaagtagagatacatcaggtgcccttcggggaaagtagagacaggtggtgcatggttgtcgtcagctcgtgtcgtg |
| | | agatgttgggttaagtcccgcaacgagcgcaacccctattgttagttgctacgcaagagcactctagcgagactgccgttgaca |
| | | aaacggaggaaggtggggacgacgtcaaatcatcatgccccttatgtcctgggctacacacgtaatacaatggcggtcaaca |
| | | gagggaggcaaagccgcgaggcagagcaaacccccaaaagccgtcccagttcggatcgcaggctgcaacccgcctgcgt |
| | | gaagtcggaatcgctagtaatcgcggatcagcatgccgcggtgaatacgttcccgggccttgtacacaccgcccgtcacacca |
| | | tgagagtcgggaacacccgaagtccgtagcctaaccgcaaggagggcgcggccgaaggtgggttcgataattggggtgaa |
| | | gtcgtaacaaggtagccgtatcggaaggtgcggctggatcacctccttt |
| Clostridium_ | SEQ ID | tagtggcggacgggtgagtaacgcgtgggtaacctgccttgtactgggggacaacagttggaaacgactgctaataccgcat |
| sp_CAG_91 | NO: 266 | aagcgcacagcttcgcatgaagcagtgtgaaaaactccggtggtacaagatggacccgcgtctgattagctggttggtgaggt |
| | | aacggcccaccaaggcgacgatcagtagccggcctgagagggtgaacggccacattgggactgagacacggcccaaactc |
| | | ctacgggaggcagcagtggggaatattgcacaatgggggaaaccctgatgcagcaacgccgcgtgagtgaagaagtatttc |
| | | ggtatgtaaagctctatcagcaggaaagaaaatgacggtacctgactaagaagccccggctaactacgtgccagcagccgcg |
| | | gtaatacgtagggggcaagcgttatccggatttactgggtgtaaagggagcgtagacggttttgcaagtctgaagtgaaagcc |
| | | cggggcttaaccccgggactgctttggaaactgtagaactagagtgcaggagaggtaagtggaattcctagtgtagcggtgaa |
| | | atgcgtagatattaggaggaacaccagtggcgaaggcggcttactggactgtaactgacgttgaggctcgaaagcgtgggga |
| | | gcaaacaggattagataccctggtagtccacgccgtaaacgatgattactaggtgttggtgggtacgacccatcggtgccgca |
| | | gcaaacgcaataagtaatccacctggggagtacgttcgcaagaatgaaactcaaaggaattgacggggacccgcacaagcg |
| | | gtggagcatgtggtttaattcgaagcaacgcgaagaaccttacctggtcttgacatccctatgaataacgggcaatgccgttagt |
| | | acttcggtacataggagacaggtggtgcatggttgtcgtcagctcgtgtcgtgagatgttgggttaagtcccgcaacgagcgca |
| | | acccttatctttagtagccagcagtaagatgggcactctagagagactgccggggataacccggaggaaggtggggatgacg |
| | | tcaaatcatcatgccccttatgaccagggctacacacgtgctacaatggcgtaaacaaagagaagcgaagtcgtgaggcaga |
| | | gcgaatctcaaaaataacgtctcagttcggattgtagtctgcaactcgactacatgaagctggaatcgctagtaatcgcagatca |
| | | gaatgctgcggtgaatacgttcccgggtcttgtacacaccgcccgtcacaccatgggagtcggaaatgcccgaagtcggtga |
| | | cctaaccgcaaggaaggagccgccgaaggcaggtctgataactggggtgaagtcgtaacaaggtagccgtatcggaaggt |
| | | gcggctggatcacctccttt |
| Ruminococcus_ | SEQ ID | cctacgggaggcagcagtggggaatattgcacaatgggggaaaccctgatgcagcgacgccgcgtgaaggaagaagtatc |
| obeum | NO: 267 | tcggtatgtaaacttctatcagcagggaagatagtgacggtacctgactaagaagccccggctaactacgtgccagcagccgc |
| | | ggtaatacgtagggggcaagcgttatccggatttactgggtgtaaagggagcgtagacggactggcaagtctgatgtgaaag |
| | | gcgggggctcaacccctggactgcattggaaactgttagtcttgagtgccggagaggtaagcggaattcctagtgtagcggtg |
| | | aaatgcgtagatattaggaggaacaccagtggcgaaggcggcttactggacggtaactgacgttgaggctcgaaagcgtgg |
| | | ggagcaaacaggattagataccctggtagtccacgccgtaaacgatgaatactaggtgttggggagcaaagctcttcggtgcc |
| | | gccgcaaacgcattaagtattccacctggggagtacgttcgcaagaatgaaactcaaaggaattgacggggacccgcacaag |
| | | cggtggagcatgtggtttaattcgaagcaacgcgaagaaccttaccaagtcttgacatccctctgaccgactcttaaccgagtct |
| | | ttccttcgggacagaggagacaggtggtgcatggttgtcgtcagctcgtgtcgtgagatgttgggttaagtcccgcaacgagcg |
| | | caacccctatccccagtagccagcggttcggccgggcactctgaggagactgccagggataacctggaggaaggcgggga |
| | | tgacgtcaaatcatcatgccccttatgatttgggctacacacgtgctacaatggcgtaaacaaagggaagcaagcctgcgaag |
| | | gtaagcaaatcccaaaaataacgtcccagttcggactgcagtctgcaactcgactgcacgaagaggaatcgctagtaatcgc |
| | | ggatcagaatgccgcggtgaatacgttcccgggtcttgtacacaccgcccgtcacaccatgggagtcagtaacgcccgaagt |
| | | cagtgacctaaccgcaagggaggagctgccgaaggcgggaccgatgactggggtgaagtcgtaacaaggtagccgtatcg |
| | | gaaggtgcggctggatcacctccttt |
| Blautia_sp. | SEQ ID | atgagagtttgatcctggctcaggatgaacgctggcggcgtgcttaacacatgcaagtcgagcgaagcaatttaagtgaagtttt |
| | NO: 268 | cggatggatcttagattgactgagcggcggacgggtgagtaacgcgtggataacctgcctcacacagggggataacagttag |
| | | aaatgactgctaataccgcataagcgcacggtaccgcatggtacagtgtgaaaaactccggtggtgtgagatggatccgcgtc |
| | | tgattaggtagttggtgaggtaacggcccaccaagccgacgatcagtagccgacctgagagggtgaccggccacattggga |
| | | ctgagacacggcccaaactcctacgggaggcagcagtggggaatattgcacaatgggcgaaagcctgatgcagcaacgcc |
| | | gcgtgagtgaagaagtatctcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgcagacggca |
| | | ctgcaagtctgaagtgaaagcccggggctcaaccccgggactgctttggaaactgtagagctagagtgctggagaggcaag |
| | | cggaattcctagtgtagcggtgaaatgcgtagatattaggaagaacaccagtggcgaaggcggcttgctggacagtaactgac |
| | | gttcaggctcgaaagcgtggggagcaaacaggattagataccctggtagtccacgccgtaaacgatgaatactaggtgttggt |
| | | gggcaaagcccatcggtgccgccgcaaacgcaataagtattccacctggggagtacgttcgcaagaatgaaactcaaagga |
| | | attgacggggacccgcacaagcggtggagcatgtggtttaattcgaagcaacgcgaagaaccttaccaagtcttgacatcgtg |
| | | atgaccggaacttaaccgttccttcccttcggggcatcatagacaggtggtgcatggttgtcgtcagctcgtgtcgtgagatgttg |
| | | ggttaagtcccgcaacgagcgcaacccttatcctcagtagccagcagtaagatgggcactctggggagactgccagggataa |
| | | cctggaggaaggtggggatgacgtcaaatcatcatgccccttatgatttgggctacacacgtgctacaatggcgtaaacaaag |
| | | ggaagcaagagggtgacctggagcaaatcccaaaaataacgtcccagttcggactgtagtctgcaacccgactacacgaagc |
| | | tggaatcgctagtaatcgcagatcagaatgctgcggtgaatacgttcccgggtcttgtacacaccgcccgtcacaccatggga |
| | | gtcagcaacgcccgaagtcagtgactcaaccgcaaggagagagctgccgaaggcggggcaggtaactggggtgaagtcgt |
| | | aacaaggtagccgtatcggaaggtgcggctggatcacctccttt |
| Blautia_sp. | SEQ ID | atgagagtttgatcctggctcaggatgaacgctggcggcgtgcttaacacatgcaagtcgagcgaagcaatttaagtgaagtttt |
| | NO: 269 | cggatggatcttagattgactgagcggcggacgggtgagtaacgcgtggataacctgcctcacacagggggataacagttag |
| | | aaatgactgctaataccgcataagcgcacggtaccgcatggtacagtgtgaaaaactccggtggtgtgagatggatccgcgtc |
| | | tgattaggtagttggtgaggtaacggcccaccaagccgacgatcagtagccgacctgagagggtgaccggccacattggga |
| | | ctgagacacggcccaaactcctacgggaggcagcagtggggaatattgcacaatgggcgaaagcctgatgcagcaacgcc |
| | | gcgtgagtgaagaagtatctcggtatgtaaagctctatcagcagggaagaaaatgacggtacctgactaagaagccccggcta |
| | | actacgtgccagcagccgcggtaatacgtagggggcaagcgttatccggatttactgggtgtaaagggagcgcagacggca |
| | | ctgcaagtctgaagtgaaagcccggggctcaaccccgggactgctttggaaactgtagagctagagtgctggagaggcaag |
| | | cggaattcctagtgtagcggtgaaatgcgtagatattaggaagaacaccagtggcgaaggcggcttgctggacagtaactgac |
| | | gttcaggctcgaaagcgtggggagcaaacaggattagataccctggtagtccacgccgtaaacgatgaatactaggtgttggt |
| | | gggcaaagcccatcggtgccgccgcaaacgcaataagtattccacctggggagtacgttcgcaagaatgaaactcaaagga |
| | | attgacggggacccgcacaagcggtggagcatgtggtttaattcgaagcaacgcgaagaaccttaccaagtcttgacatcgtg |
| | | atgaccggaacttaaccgttccttcccttcggggcatcatagacaggtggtgcatggttgtcgtcagctcgtgtcgtgagatgttg |
| | | ggttaagtcccgcaacgagcgcaacccttatcctcagtagccagcagtaagatgggcactctgtggagactgccagggataa |
| | | cctggaggaaggtggggatgacgtcaaatcatcatgccccttatgatttgggctacacacgtgctacaatggcgtaaacaaag |
| | | ggaagcaagagggtgacctggagcaaatcccaaaaataacgtcccagttcggactgtagtctgcaacccgactacacgaagc |
| | | tggaatcgctagtaatcgcagatcagaatgctgcggtgaatacgttcccgggtcttgtacacaccgcccgtcacaccatggga |
| | | gtcagcaacgcccgaagtcagtgactcaaccgcaaggagagagctgccgaaggcggggcaggtaactggggtgaagtcgt |
| | | aacaaggtagccgtatcggaaggtgcggctggatcacctccttt |
| Lachnospira_ | SEQ ID | ttatgagagtttgatcctggctcaggatgaacgctggcggcgtgcttaacacatgcaagtcgaacgaagcatttgcgacagattt |
| pectinoschiza | NO: 270 | cttcggattgaagttgcttatgactgagtggcggacgggtgagtaacgcgtgggtaacctgcctcacacagggggatagcagt |
| | | tggaaacggctgataataccgcataagcgcacagtaccgcatggtacagtgtgaaaaactccggtggtgtgagatggacccg |
| | | cgtctgattagcttgttggcagggtaacggcctaccaaggcaacgatcagtagccgacctgagagggtgaccggccacattg |
| | | ggactgagacacggcccagactcctacgggaggcagcagtggggaatattgcacaatggaggaaactctgatgcagcgac |
| | | gccgcgtgagtgaagaagtaattcgttatgtaaagctctatcagcagggaagatagtgacggtacctgactaagaagctccgg |
| | | ctaaatacgtgccagcagccgcggtaatacgtatggagcaagcgttatccggatttactgggtgtaaagggagtgtaggtggc |
| | | atcacaagtcagaagtgaaagcccggggctcaaccccgggactgcttttgaaactgtggagctggagtgcaggagaggcaa |
| | | gtggaattcctagtgtagcggtgaaatgcgtagatattaggaggaacaccagtggcgaaggcggcttgctggactgtaactga |
| | | cactgaggctcgaaagcgtggggagcaaacaggattagataccctggtagtccacgccgtaaacgatgaatactaggtgtcg |
| | | gggctcataagagcttcggtgccgcagcaaacgcaataagtattccacctggggagtacgttcgcaagaatgaaactcaaag |
| | | gaattgacggggacccgcacaagcggtggagcatgtggtttaattcgaagcaacgcgaagaaccttaccaagtcttgacatcc |
| | | tcttgcccggtcagtaatgtgaccttttcttcggaacaagagtgacaggtggtgcatggttgtcgtcagctcgtgtcgtgagatgtt |
| | | gggttaagtcccgcaacgagcgcaacccctattcttagtagccagcatttaaggtgggcactctaggaagactgccagggata |
| | | acctggaggaaggtggggatgacgtcaaatcatcatgccccttatgacttgggctacacacgtgctacaatggcgtaaacaaa |
| | | gtgaagcgagagtgtgagcttaagcaaatcacaaaaataacgtctcagttcggattgtagtctgcaactcgactacatgaagct |
| | | ggaatcgctagtaatcgcgaatcagaatgtcgcggtgaatacgttcccgggtcttgtacacaccgcccgtcacaccatgggag |
| | | tcggaaatgcccgaagtcggtgacctaacgaaagaaggagccgccgaaggcaggtctgataactggggtgaagtcgtaaca |
| | | aggtagccgtatcggaaggtgcggctggatcacctccttt |
| Lactonifactor_ | SEQ ID | ctgagagggtgaccggccacattgggactgagacacggcccaaactcctacgggaggcagcagtggggaatattgcacaat |
| longoviformis | NO: 271 | gggggaaaccctgatgcagcgacgccgcgtgaaggaagaagtatttcggtatgtaaacttctatcagcagggaagaaaatga |
| | | cggtacctgactaagaagccccggctaattacgtgccagcagccgcggtaatacgtaaggggcaagcgttatccggatttact |
| | | gggtgtaaagggagcgtagacggcagtgcaagtctgatgtgaaagcccggggctcaaccccgggactgcattggaaactgt |
| | | gcagctagagtgtcggagaggtaagtggaattcctagtgtagcggtgaaatgcgtagatattaggaggaacaccagtggcga |
| | | aggcggcttactggacgataactgacgttgaggctcgaaagcgtggggagcaaacaggattagataccctggtagtccacgc |
| | | cgtaaacgatgaatactaggtgtcgggcgccaaaggcgttcggtgccgcagcaaacgcaataagtattccacctggggagta |
| | | cgttcgcaagaatgaaactcaaaggaattgacggggacccgcacaagcggtggagcatgtggtttaattcgaagcaacgcga |
| | | agaaccttaccaagtcttgacatctgcctgaccggtccgtaacaggacccttccttcgggacaggcaagacaggtggtgcatg |
| | | gttgtcgtcagctcgtgtcgtgagatgttgggttaagtcccgcaacgagcgcaacccttatccttagtagccagcaggtagagct |
| | | gggcactctagggagactgccagggacaacctggaggaaggtggggatgacgtcaaatcatcatgccccttatgatttgggc |
| | | tacacacgtgctacaatggcgtaaacaaagggaggcgaaggggtgacctggagcaaatcccaaaaataacgtctcagttcgg |
| | | attgtagtctgcaactcgactacatgaagctggaatcgctagtaatcgcgaatcagaatgtcgcggtgaatacgttcccgggtct |
| | | tgtacacaccgcccgtcacaccatgggagtcggatatgcccgaagccggtgaccgaacccgaaagggaaggagccgtcga |
| | | aggtggagccgataactggggtgaagtcgtaacaaggtagccgtatcggaaggtgcggctggatcacctccttt |
| Eubacterium_ | SEQ ID | aacgagagtttgatcctggctcaggatgaacgctggcggcgtgcttaacacatgcaagtcgagcgaagcgctttacttagattt |
| contortum | NO: 272 | cttcggattgaagagttttgcgactgagcggcggacgggtgagtaacgcgtgggtaacctgcctcatacagggggataacagt |
| | | tagaaatgactgctaataccgcataagaccacagtaccgcatggtaccgtgggaaaaactccggtggtatgagatggacccgc |
| | | gtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgaaggatgaagtatttcggtatgtaaacttctatcagcagggaagaaaatgacggtacctgactaagaagccccggc |
| | | taactacgtgccagcagccgcggtaatacgtagggggcaagcgttatccggatttactgggtgtaaagggagcgtagacggtt |
| | | atgtaagtctgatgtgaaaacccggggctcaaccccgggactgcattggaaactatgtaactagagtgtcggagaggtaagtg |
| | | gaattcctagtgtagcggtgaaatgcgtagatattaggaggaacaccagtggcgaaggcggcttactggacgatgactgacgt |
| | | tgaggctcgaaagcgtggggagcaaacaggattagataccctggtagtccacgccgtaaacgatgcatactaggtgtcgggt |
| | | ggcaaagccattcggtgccgcagcaaacgcaataagtatgccacctggggagtacgttcgcaagaatgaaactcaaaggaat |
| | | tgacggggacccgcacaagcggtggagcatgtggtttaattcgaagcaacgcgaagaaccttacctgctcttgacatccccct |
| | | gaccggcgcgtaatggtgcctttccttcgggacaggggagacaggtggtgcatggttgtcgtcagctcgtgtcgtgagatgttg |
| | | ggttaagtcccgcaacgagcgcaacccttatctttagtagccagcggttcggccgggcactctagagagactgccagggataa |
| | | cctggaggaaggtggggatgacgtcaaatcatcatgccccttatgagcagggctacacacgtgctacaatggcgtaaacaaa |
| | | gggaggcgaagccgcgaggtggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gttggtaacgcccgaagtcagtgacccaaccgtaaggagggagctgccgaaggtgggaccgataactggggtgaagtcgta |
| | | acaaggtagccgtatcggaaggtgcggctggatcacctccttt |
| Eubacterium_ | SEQ ID | aacgagagtttgatcctggctcaggatgaacgctggcggcgtgcttaacacatgcaagtcgagcgaagcgctttacttagattt |
| contortum | NO: 273 | cttcggattgaagagttttgcgactgagcggcggacgggtgagtaacgcgtgggtaacctgcctcatacagggggacaacag |
| | | ttagaaatgactgctaataccgcataagaccacggtaccgcatggtacagtgggaaaaactccggtggtatgagatggacccg |
| | | cgtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgaaggatgaagtatttcggtatgtaaacttctatcagcagggaagaaaatgacggtacctgactaagaagccccggc |
| | | taactacgtgccagcagccgcggtaatacgtagggggcaagcgttatccggatttactgggtgtaaagggagcgtagacggtt |
| | | atgtaagtctgatgtgaaaacccggggctcaaccccgggactgcattggaaactatgtaactagagtgtcggagaggtaagtg |
| | | gaattcctagtgtagcggtgaaatgcgtagatattaggaggaacaccagtggcgaaggcggcttactggacgatgactgacgt |
| | | tgaggctcgaaagcgtggggagcaaacaggattagataccctggtagtccacgccgtaaacgatgcatactaggtgtcgggt |
| | | ggcaaagccattcggtgccgcagcaaacgcaataagtatgccacctggggagtacgttcgcaagaatgaaactcaaaggaat |
| | | tgacggggacccgcacaagcggtggagcatgtggtttaattcgaagcaacgcgaagaaccttacctgctcttgacatccccct |
| | | gaccggcgcgtaatggtgcctttccttcgggacaggggagacaggtggtgcatggttgtcgtcagctcgtgtcgtgagatgttg |
| | | ggttaagtcccgcaacgagcgcaacccttatctttagtagccagcggttcggccgggcactctagagagactgccagggataa |
| | | cctggaggaaggtggggatgacgtcaaatcatcatgccccttatgagcagggctacacacgtgctacaatggcgtaaacaaa |
| | | gggaggcgaagccgcgaggtggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gttggtaacgcccgaagtcagtgacccaaccgtaaggagggagctgccgaaggtgggaccgataactggggtgaagtcgta |
| | | acaaggtagccgtatcggaaggtgcggctggatcacctccttt |
| Eubacterium_ | SEQ ID | aacgagagtttgatcctggctcaggatgaacgctggcggcgtgcttaacacatgcaagtcgagcgaagcgctttacttagattt |
| contortum | NO: 274 | cttcggattgaagagttttgcgactgagcggcggacgggtgagtaacgcgtgggtaacctgcctcatacagggggataacagt |
| | | tagaaatgactgctaataccgcataagaccacggtaccgcatggtacagtgggaaaaactccggtggtatgagatggacccg |
| | | cgtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgaaggatgaagtatttcggtatgtaaacttctatcagcagggaagaaaatgacggtacctgactaagaagccccggc |
| | | taactacgtgccagcagccgcggtaatacgtagggggcaagcgttatccggatttactgggtgtaaagggagcgtagacggtt |
| | | atgtaagtctgatgtgaaaacccggggctcaaccccgggactgcattggaaactatgtaactagagtgtcggagaggtaagtg |
| | | gaattcctagtgtagcggtgaaatgcgtagatattaggaggaacaccagtggcgaaggcggcttactggacgatgactgacgt |
| | | tgaggctcgaaagcgtggggagcaaacaggattagataccctggtagtccacgccgtaaacgatgcatactaggtgtcgggt |
| | | ggcaaagccattcggtgccgcagcaaacgcaataagtatgccacctggggagtacgttcgcaagaatgaaactcaaaggaat |
| | | tgacggggacccgcacaagcggtggagcatgtggtttaattcgaagcaacgcgaagaaccttacctgctcttgacatccccct |
| | | gaccggcgcgtaatggtgcctttccttcgggacaggggagacaggtggtgcatggttgtcgtcagctcgtgtcgtgagatgttg |
| | | ggttaagtcccgcaacgagcgcaacccttatctttagtagccagcggttcggccgggcactctagagagactgccagggataa |
| | | cctggaggaaggtggggatgacgtcaaatcatcatgccccttatgagcagggctacacacgtgctacaatggcgtaaacaaa |
| | | gggaggcgaagccgcgaggtggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gttggtaacgcccgaagtcagtgacccaaccgtaaggagggagctgccgaaggtgggaccgataactggggtgaagtcgta |
| | | acaaggtagccgtatcggaaggtgcggctggatcacctccttt |
| Eubacterium_ | SEQ ID | aacgagagtttgatcctggctcaggatgaacgctggcggcgtgcttaacacatgcaagtcgagcgaagcactttacttagatttc |
| contortum | NO: 275 | ttcggaatgaagagtgttgtgactgagcggcggacgggtgagtaacgcgtgggtaacctgcctcatacagggggataacagt |
| | | tagaaatgactgctaataccgcataagaccacggtaccgcatggtacagtgggaaaaactccggtggtatgagatggacccg |
| | | cgtctgattagctagttggtaaggtaacggcttaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgaaggatgaagtatttcggtatgtaaacttctatcagcagggaagaaaatgacggtacctgactaagaagccccggc |
| | | taactacgtgccagcagccgcggtaatacgtagggggcaagcgttatccggatttactgggtgtaaagggagcgtagacggtt |
| | | atgtaagtctgatgtgaaaacccggggctcaaccccgggactgcattggaaactatgtaactagagtgtcggagaggtaagtg |
| | | gaattcctagtgtagcggtgaaatgcgtagatattaggaggaacaccagtggcgaaggcggcttactggacgatgactgacgt |
| | | tgaggctcgaaagcgtggggagcaaacaggattagataccctggtagtccacgccgtaaacgatgcatactaggtgtcgggt |
| | | ggcaaagccattcggtgccgcagcaaacgcaataagtatgccacctggggagtacgttcgcaagaatgaaactcaaaggaat |
| | | tgacggggacccgcacaagcggtggagcatgtggtttaattcgaagcaacgcgaagaaccttacctgctatgacatccccct |
| | | gaccggcgcgtaatggtgcctttccttcgggacaggggagacaggtggtgcatggttgtcgtcagctcgtgtcgtgagatgttg |
| | | ggttaagtcccgcaacgagcgcaacccttatctttagtagccagcggttcggccgggcactctagagagactgccagggataa |
| | | cctggaggaaggtggggatgacgtcaaatcatcatgccccttatgagcagggctacacacgtgctacaatggcgtaaacaaa |
| | | gggaggcgaagccgcgaggtggagcaaatcccaaaaataacgtctcagttcggattgtagtctgcaactcgactacatgaag |
| | | ctggaatcgctagtaatcgcgaatcagaatgtcgcggtgaatacgttcccgggtcttgtacacaccgcccgtcacaccatggga |
| | | gttggtaacgcccgaagtcagtgacccaaccgtaaggagggagctgccgaaggtgggaccgataactggggtgaagtcgta |
| | | acaaggtagccgtatcggaaggtgcggctggatcacctccttt |
| Eisenbergiella_ | SEQ ID | ggagaggtaagcggaattcctagtgtagcggtgaaatgcgtagatattaggaggaacaccagtggcgaaggcggcttactgg |
| tayi | NO: 276 | actgtaactgacgttgaggctcgaaagcgtggggagcaaacaggattagataccctggtagtccacgcggtaaacgatgattg |
| | | ctaggtgtaggtgggtatggacccatcggtgccgcagctaacgcaataagcaatccacctggggagtacgttcgcaagaatg |
| | | aaactcaaaggaattgacggggacccgcacaagcggtggagcatgtggtttaattcgaagcaacgcgaagaaccttaccaag |
| | | tcttgacatccccctgaatatctcgtaaaagggataggccttcgggacaggggagacaggtggtgcatggttgtcgtcagctcg |
| | | tgtcgtgagatgttgggttaagtcccgcaacgagcgcaacccttattcttagtagccagcaggtaaagctgggcactctaagga |
| | | gactgccggggataacccggaggaaggcggggatgacgtcaaatcatcatgccccttatgatttgggctacacacgtgctac |
| | | aatggcgtaaacaaagggaagcgagacagtgatgtggagcaaatcccagaaataacgtctcagttcggattgtagtctgcaac |
| | | tcgactacatgaagctggaatcgctagtaatcgcgaatcagcatgtcgcggtgaatacgttcccgggtcttgtacacaccgccc |
| | | gtcacaccatgggagttggaaatgcccgaagtctgtgacctaaccgaaagggaggagcagccgaaggcaggtctgataact |
| | | ggggtgaagtcgtaacaaggtagccgtatcggaaggtgcggctggatcacctccttt |
| Ruminococcus_ | SEQ ID | aacgagagtttgatcctggctcaggatgaacgctggcggcgtgcttaacacatgcaagtcgagcgaagcgcttttgcggatttc |
| gnavus_sp | NO: 277 | ttcggattgaagcaactgtgactgagcggcggacgggtgagtaacgcgtgggtaacctgcctcatacagggggataacagtt |
| | | ggaaacggctgctaataccgcataagcgcacagtaccgcatggtaccgtgtgaaaaactccggtggtatgagatggacccgc |
| | | gtctgattagctagttggtggggtaacggcctaccaaggcgacgatcagtagccgacctgagagggtgaccggccacattgg |
| | | gactgagacacggcccaaactcctacgggaggcagcagtggggaatattgcacaatgggggaaaccctgatgcagcgacg |
| | | ccgcgtgagcgatgaagtatttcggtatgtaaagctctatcagcagggaagaaaatgacggtacctgactaagaagccccggc |
| | | taactacgtgccagcagccgcggtaatacgtagggggcaagcgttatccggatttactgggtgtaaagggagcgtagacggc |
| | | atggcaagccagatgtgaaagcccggggctcaaccccgggactgcatttggaactgtcaggctagagtgtcggagaggtaa |
| | | gcggaattcctagtgtagcggtgaaatgcgtagatattaggaggaacaccagtggcgaaggcggcttactggacgatgactg |
| | | acgttgaggctcgaaagcgtggggagcaaacaggattagataccctggtagtccacgccgtaaacgatgaatactaggtgtc |
| | | gggtggcaaagccattcggtgccgcagcaaacgcaataagtattccacctggggagtacgttcgcaagaatgaaactcaaag |
| | | gaattgacggggacccgcacaagcggtggagcatgtggtttaattcgaagcaacgcgaagaaccttacctggtcttgacatcc |
| | | ctatgaccgctctttaatcggagctttccttcgggacagaggagacaggtggtgcatggttgtcgtcagctcgtgtcgtgagatg |
| | | ttgggttaagtcccgcaacgagcgcaacccctatctttagtagccagcattttggatgggcactctagagagactgccagggat |
| | | aacctggaggaaggtggggatgacgtcaaatcatcatgccccttatgaccagggctacacacgtgctacaatggcgtaaacaa |
| | | agggaagcgaacctgcgagggggagcaaatcccaaaaataacgtctcagttcggattgtagtctgcaactcgactacatgaa |
| | | gctggaatcgctagtaatcgcgaatcagaatgtcgcggtgaatacgttcccgggtcttgtacacaccgcccgtcacaccatgg |
| | | gagtcagtaacgcccgaagtcagtgacccaaccgtaaggagggagctgccgaaggtgggaccgataactggggtgaagtc |
| | | gtaacaaggtagccgtatcggaaggtgcggctggatcacctccttt |
| Clostridium_ | SEQ ID | tttgagagtttgatcctggctcaggatgaacgctggcggcgtgcctaacacatgcaagtcgaacgaagcgattaggatgaagtt |
| aldenense_ | NO: 278 | ttcggatggattcctggttgactgagtggcggacgggtgagtaacgcgtggataacctgcctcacactgggggataacagtta |
| sp | | gaaatggctgctaataccgcataagcgcacagtaccgcatggtacggtgtgaaaaacccaggtggtgtgagatggatccgcg |
| | | tctgattagccagttggcggggtaacggcccaccaaagcgacgatcagtagccgacctgagagggtgaccggccacattgg |
| | | gactgagacacggcccaaactcctacgggaggcagcagtggggaatattgcacaatgggcggaagcctgatgcagcgacg |
| | | ccgcgtgagtgaagaagtatctcggtatgtaaagctctatcagcagggaagaaaatgacggtacctgactaagaagccccgg |
| | | ctaactacgtgccagcagccgcggtaatacgtagggggcaagcgttatccggatttactgggtgtaaagggagcgtagacgg |
| | | cgacgcaagtctggagtgaaagcccggggcccaaccccgggactgctttggaaactgtgctgctggagtgcaggagaggta |
| | | agtggaattcctagtgtagcggtgaaatgcgtagatattaggaggaacaccagtggcgaaggcggcttactggactgtaactg |
| | | acgttgaggctcgaaagcgtggggagcaaacaggattagataccctggtagtccacgccgtaaacgatgaatgctaggtgtc |
| | | ggggggcaaagcccttcggtgccgccgctaacgcaataagcattccacctggggagtacgttcgcaagaatgaaactcaaag |
| | | gaattgacggggacccgcacaagcggtggagcatgtggtttaattcgaagcaacgcgaagaaccttaccaagtcttgacatcc |
| | | ccctgaccggcgcgtaacggtgcccttccttcgggacaggggagacaggtggtgcatggttgtcgtcagctcgtgtcgtgag |
| | | atgttgggttaagtcccgcaacgagcgcaacccttatccttagtagccagcacgtcgtggtgggcactctagggagactgcca |
| | | gggataacctggaggaaggtggggatgacgtcaaatcatcatgccccttatgatttgggctacacacgtgctacaatggcgtaa |
| | | acaaagggaggcgaccctgcgaaggtaagcaaatcccaaaaataacgtcccagttcggactgtagtctgcaacccgactaca |
| | | cgaagctggaatcgctagtaatcgcggatcagaatgccgcggtgaatacgttcccgggtcttgtacacaccgcccgtcacacc |
| | | atgggagtcagcaacgcccgaagtcagtgacccaaccttaacaggagggagctgccgaaggcggggcaggtaactggggt |
| | | gaagtcgtaacaaggtagccgtatcggaaggtgcggctggatcacctccttt |
| Clostridium_ | SEQ ID | acgagagtttgatcctggctcaggacgaacgctggcggcgtgcctaacacatgcaagtcgaacgagaatctttgaacagatct |
| sp | NO: 279 | tttcggagtgacgttcaaagaggaaagtggcggacgggcgagtaacgcgtgagtaacctgcccataagagggggataatcc |
| | | atggaaacgtggactaataccgcatattgtagttaagttgcatgacttgattatgaaagatttatcgcttatggatggactcgcgtc |
| | | agattagatagttggtgaggtaacggctcaccaagtcaacgatctgtagccgaactgagaggttgatcggccgcattgggact |
| | | gagacacggcccagactcctacgggaggcagcagtggggaatattgcgcaatgggggcaaccctgacgcagcaacgccg |
| | | cgtgcaggaagaaggtcttcggattgtaaactgttgtcgcaagggaagaagacagtgacggtaccttgtgagaaagtcacgg |
| | | ctaactacgtgccagcagccgcggtaatacgtaggtgacaagcgttgtccggatttactgggtgtaaagggcgcgtaggcgg |
| | | actgtcaagtcagtcgtgaaataccggggcttaaccccggggctgcgattgaaactgacagccttgagtatcggagaggaaa |
| | | gcggaattcctagtgtagcggtgaaatgcgtagatattaggaggaacaccagtggcgaaggcggctttctggacgacaactg |
| | | acgctgaggcgcgaaagtgtggggagcaaacaggattagataccctggtagtccacaccgtaaacgatggatactaggtgta |
| | | ggaggtatcgaccccttctgtgccgcagttaacacaataagtatcccacctggggagtacgaccgcaaggttgaaactcaaag |
| | | gaattgacgggggcccgcacaagcagtggagtatgtggtttaattcgaagcaacgcgaagaaccttacctgggcttgacatcc |
| | | ctggaatcgagtagagatacttgagtgccttcgggaatcaggtgacaggtggtgcatggttgtcgtcagctcgtgtcgtgagat |
| | | gttgggttaagtcccgcaacgagcgcaacccctattgtcagttgccatcattaagttgggcactctggcgagactgccggtgac |
| | | aaatcggaggaaggtggggacgacgtcaaatcatcatgccccttatgcccagggctacacacgtactacaatggccgataac |
| | | aaagtgcagcgaaaccgtgaggtggagcgaatcacaaaactcggtctcagttcagattgcaggctgcaactcgcctgcatga |
| | | agttggaattgctagtaatcgcggatcagaatgccgcggtgaatacgttcccgggccttgtacacaccgcccgtcacaccatga |
| | | gagtcgataacacccgaagcctgtgagctaacctttaggaggcagcagtcgaaggtggggttgatgattggggtgaagtcgta |
| | | acaaggtagccgtatcggaaggtgcggctggatcacctccttt |
| Clostridium_ | SEQ ID | tttgagagtttgatcctggctcaggatgaacgctggcggcgtgcctaacacatgcaagtcgaacgaagcatttcagatgaagttt |
| lavalense | NO: 280 | tcggatggattctgagatgactgagtggcggacgggtgagtaacacgtggataacctgcctcacactgggggacaacagtta |
| | | gaaatgactgctaataccgcataagcgcacagtaccgcatggtacagtgtgaaaaactccggtggtgtgagatggatccgcgt |
| | | ctgattagccagttggcggggtaacggcccaccaaagcgacgatcagtagccgacctgagagggtgaccggccacattggg |
| | | actgagacacggcccaaactcctacgggaggcagcagtggggaatattgcacaatgggcgaaagcctgatgcagcgacgc |
| | | cgcgtgagtgaagaagtatttcggtatgtaaagctctatcagcagggaagataatgacggtacctgactaagaagccccggct |
| | | aactacgtgccagcagccgcggtaatacgtagggggcaagcgttatccggatttactgggtgtaaagggagcgtagacggca |
| | | tggcaagtctgaagtgaaaacccagggctcaaccctgggactgctttggaaactgtcaagctagagtgcaggagaggtaagt |
| | | ggaattcctagtgtagcggtgaaatgcgtagatattaggaggaacaccagtggcgaaggcggcttactggactgtaactgacg |
| | | ttgaggctcgaaagcgtggggagcaaacaggattagataccctggtagtccacgccgtaaacgatgagtgctaggtgttggg |
| | | gggcaaagcccttcggtgccgtcgcaaacgcaataagcactccacctggggagtacgttcgcaagaatgaaactcaaagga |
| | | attgacggggacccgcacaagcggtggagcatgtggtttaattcgaagcaacgcgaagaaccttaccaagtcttgacatcctct |
| | | tgaccggcgtgtaacggcgcctttccttcgggacaagggagacaggtggtgcatggttgtcgtcagctcgtgtcgtgagatgtt |
| | | gggttaagtcccgcaacgagcgcaacccttatccttagtagccagcattaagatgggcactctagggagactgccagggaca |
| | | acctggaggaaggtggggatgacgtcaaatcatcatgccccttatgatttgggctacacacgtgctacaatggcgtaaacaaa |
| | | gggaagcgaccctgcgaaggtgagcaaatctcaaaaataacgtcccagttcggactgtagtctgcaacccgactacacgaag |
| | | ctggaatcgctagtaatcgcgaatcagaatgtcgcggtgaatacgttcccgggtcttgtacacaccgcccgtcacaccatggga |
| | | gtcagcaacgcccgaagtcagtgacccaactcgcaagagagggagctgccgaaggcggggcaggtaactggggtgaagt |
| | | cgtaacaaggtagccgtatcggaaggtgcggctggatcacctccttt |
| Blautia_producta | SEQ ID | AGAGTTTGATCCTGGCTCAGGATGAACGCTGGCGGCGTGCTTAACACATGCA |
| | NO: 281 | AGTCGAGCGAAGCACTAAGACGGATTTCTTCGGATTGAAGTCTTTGTGACTG |
| | | AGCGGCGGACGGGTGAGTAACGCGTGGGTAACCTGCCTCATACAGGGGGAT |
| | | AACAGTTAGAAATGACTGCTAATACCGCATAAGCGCACAGGACCGCATGGT |
| | | CTGGTGTGAAAAACTCCGGTGGTATGAGATGGACCCGCGTCTGATTAGCTAG |
| | | TTGGAGGGGTAACGGCCCACCAAGGCGACGATCAGTAGCCGGCCTGAGAGG |
| | | GTGAACGGCCACATTGGGACTGAGACACGGCCCAGACTCCTACGGGAGGCA |
| | | GCAGTGGGGAATATTGCACAATGGGGGAAACCCTGATGCAGCGACGCCGCG |
| | | TGAAGGAAGAAGTATCTCGGTATGTAAACTTCTATCAGCAGGGAAGAAAAT |
| | | GACGGTACCTGACTAAGAAGCCCCGGCTAACTACGTGCCAGCAGCCGCGGT |
| | | AATACGTAGGGGGCAAGCGTTATCCGGATTTACTGGGTGTAAAGGGAGCGT |
| | | AGACGGAAGAGCAAGTCTGATGTGAAAGGCTGGGGCTTAACCCCAGGACTG |
| | | CATTGGAAACTGTTGTTCTAGAGTGCCGGAGAGGTAAGCGGAATTCCTAGTG |
| | | TAGCGGTGAAATGCGTAGATATTAGGAGGAACACCAGTGGCGAAGGCGGCT |
| | | TACTGGACGGTAACTGACGTTGAGGCTCGAAAGCGTGGGGAGCAAACAGGA |
| | | TTAGATACCCTGGTAGTCCACGCCGTAAACGATGAATACTAGGTGTCGGGTG |
| | | GCAAAGCCATTCGGTGCCGCAGCAAACGCAATAAGTATTCCACCTGGGGAG |
| | | TACGTTCGCAAGAATGAAACTCAAAGGAATTGACGGGGACCCGCACAAGCG |
| | | GTGGAGCATGTGGTTTAATTCGAAGCAACGCGAAGAACCTTACCAAGTCTTG |
| | | ACATCCCTCTGACCGTCCCGTAACGGGGACTTCCCTTCGGGGCAGAGGAGAC |
| | | AGGTGGTGCATGGTTGTCGTCAGCTCGTGTCGTGAGATGTTGGGTTAAGTCC |
| | | CGCAACGAGCGCAACCCTTATCCTTAGTAGCCAGCACATGATGGTGGGCACT |
| | | CTAGGGAGACTGCCGGGGATAACCCGGAGGAAGGCGGGGACGACGTCAAA |
| | | TCATCATGCCCCTTATGATTTGGGCTACACACGTGCTACAATGGCGTAAACA |
| | | AAGGGAAGCGAGACAGCGATGTTGAGCGAATCCCAAAAATAACGTCCCAGT |
| | | TCGGACTGCAGTCTGCAACTCGACTGCACGAAGCTGGAATCGCTAGTAATCG |
| | | CGGATCAGAATGCCGCGGTGAATACGTTCCCGGGTCTTGTACACACCGCCCG |
| | | TCACACCATGGGAGTCAGTAACGCCCGAAGTCAGTGACCTAACCGAAAGGA |
| | | AGGAGCTGCCGAAGGCGGGACCGATAACTGGGGTGAAGTCGTAACAAGGTA |
| | | ACC |
| Clostridium_ | SEQ ID | NNNNNNNGAGATTTGATCCTGGCTCAGGATGAACGCTGGCCGGCCGTGCTT |
| nexile | NO: 282 | ACACATGCAGTCGAACGAAGCGCTTAAACTGGATTTCTTCGGATTGAAGTTT |
| | | TTGCTGACTGAGTGGCGGACGGGTGAGTAACGCGTGGGTAACCTGCCTCAT |
| | | ACAGGGGGATAACAGTTAGAAATGACTGCTAATACCNNATAAGCGCACAGT |
| | | GCTGCATGGCACAGTGTAAAAACTCCGGTGGTATGAGATGGACCCGCGTCT |
| | | GATTAGCTAGTTGGTGGGGTAACGGCCTACCAAGGCGACGATCAGTAGCCG |
| | | GCCTGAGAGGGTGAACGGCCACATTGGGACTGAGACACGGNCCAAACTCCT |
| | | ACGGGAGGCAGCAGTGGGGAATATTGCACAATGGGGGAAACCCTGATGCAG |
| | | CGACGCCGCGTGAGCGAAGAAGTATTTNGGTATGTAAAGCTCTATCAGCAG |
| | | GGAAGAAAATGACGGTACCTGACTAAGAAGCACCGGCTAAATACGTGCCAG |
| | | CAGCCGCGGTAATACGTAGGTGCAAGCGTTATCCGGATTTACTGGGTGTAAA |
| | | GGGAGCGTAGACGGTTGTGTAAGTCTGATGTGAAAGCCCGGGGCTCAACCC |
| | | CGGACTGCATTGGAAACTATGTAACTAGAGTGTCGGAGAGGTAAGCGGAAT |
| | | TCCTAGTGTAGCGGTGAAATGCGTAGATATTAGGAGGAACACCAGTGGCGA |
| | | AGGCGGCTTACTGGACGATCACTGACGTTGAGGCTCGAAAGCGTGGGGAGC |
| | | AAACAGGATTAGATACCCTGGTAGTCCACGCCGTAAACGATGACTACTAGG |
| | | TGTCGGGGAGCAAAGCTCTTCGGTGCCGCAGCAAACGCAATAAGTAGTCCA |
| | | CCTGGGGAGTACGTTCGCAAGAATGAAACTCAAAGGAATTGACGGGGACCC |
| | | GCACAAGCGTGGAGCATGTGGTTTAATTCGAGCAACGCGAAGACCTTACCT |
| | | GGTCTTGACATCCCGGTGACCGGTCCAGTAATGGGACCTTTCCTTCGGGACA |
| | | CGGTGACAGGTGGTGCATGGTTGTCGTCAGCTCGTGTCGTGAGATGTTGGGT |
| | | TAAGTCCCGCAACGAGCGCAACCCCTATCTTCAGTAGCCAGCATTTAAGGTG |
| | | GGCACTCTGGAGAGACTGCCAGGGATAACCTGGAGGAAGGTGGGGATGACG |
| | | TCAAATCATCATGCCCCTTATGACCAGGGCTACACACGTGCTACAATGGCGT |
| | | AAACAAAGGGAAGCGAACCTGTGAGGGGAAGCAAATCTCAAAAATAACGT |
| | | CTCAGTTCGGATTGTAGTCTGCAACTCGACTACATGAAGCTGGAATCGCTAG |
| | | TAATCGCGAATCAGCATGTCGCGGTGAATACGTTCCCGGGTCTTGTACACAC |
| | | CGCCCGTCACACCATGGGAGTCAGTAACGCCCGAAGTCAGTGACCCAACCG |
| | | TAAGGAGGGAGCTGCCGAAGGTGGGACCGATAACTGGGGTGAAGTCGTAAC |
| | | AAGGTAGCCGTATCGGAAGGTGCGGCTGGATCACCTCC |
| Roseburia_ | SEQ ID | TTGATCCTGGCTCAGGATGAACGCTGGCGGCGTGCTTAACACATGCAAGTCG |
| inulinivorans | NO: 283 | AACGAAGCACTTTTACAGATTTCTTCGGAATGAAGTTTTAGTGACTGAGTGG |
| | | CGGACGGGTGAGTAACGCGTGGGTAACCTGCCTCACACAGGGGGATAACAG |
| | | TTGGAAACGGCTGCTAATACCGCATAAGCGCACAGTACCGCATGGTACAGT |
| | | GTGAAAAACTCCGGTGGTGTGAGATGGACCCGCGTCTGATTAGCTAGTTGGC |
| | | AGGGCAACGGCCTACCAAGGCGACGATCAGTAGCCGACCTGAGAGGGTGAC |
| | | CGGCCACATTGGGACTGAGACACGGCCCAAACTCCTACGGGAGGCAGCAGT |
| | | GGGGAATATTGCACAATGGGGGAAACCCTGATGCAGCGACGCCGCGTGAGC |
| | | GAAGAAGTATTTCGGTATGTAAAGCTCTATCAGCAGGGAAGAAGAAATGAC |
| | | GGTACCTGACTAAGAAGCACCGGCTAAATACGTGCCAGCAGCCGCGGTAAT |
| | | ACGTATGGTGCAAGCGTTATCCGGATTTACTGGGTGTAAAGGGAGCGCAGG |
| | | CGGAAGGCTAAGTCTGATGTGAAAGCCCGGGGCTCAACCCCGGTACTGCAT |
| | | TGGAAACTGGTCATCTAGAGTGTCGGAGGGGTAAGTGGAATTCCTAGTGTA |
| | | GCGGTGAAATGCGTAGATATTAGGAGGAACACCAGTGGCGAAGGCGGCTTA |
| | | CTGGACGATAACTGACGCTGAGGCTCGAAAGCGTGGGGAGCAAACAGGATT |
| | | AGATACCCTGGTAGTCCACGCCGTAAACGATGAATACTAGGTGTCGGAAAG |
| | | CACAGCTTTTCGGTGCCGCCGCAAACGCATTAAGTATTCCACCTGGGGAGTA |
| | | CGTTCGCAAGAATGAAACTCAAAGGAATTGACGGGGACCCGCACAAGCGGT |
| | | GGAGCATGTGGTTTAATTCGAAGCAACGCGAAGAACCTTACCAAGTCTTGA |
| | | CATCCTTCTGACCGGACAGTAATGTGTCCTTTCCTTCGGGACAGAAGTGACA |
| | | GGTGGTGCATGGTTGTCGTCAGCTCGTGTCGTGAGATGTTGGGTTAAGTCCC |
| | | GCAACGAGCGCAACCCTTATCCCCAGTAGCCAGCGGTTCGGACGGGCACTC |
| | | TGAGGAGACTGCCAGGGATAACCTGGAGGAAGGTGGGGATGACGTCAAATC |
| | | ATCATGCCCCTTATGACTTGGGCTACACACGTGCTACAATGGCGTAAACAAA |
| | | GGGAAGCGAGACCGTGAGGTGGAGCAAATCCCAAAAATAACGTCTCAGTTC |
| | | GGACTGTAGTCTGCAACCCGACTACACGAAGCTGGAATCGCTAGTAATCGC |
| | | AGATCAGAATGCTGCGGTGAATACGTTCCCGGGTCTTGTACACACCGCCCGT |
| | | CACACCATGGGAGTTGGAAATGCCCGAAGTCAGTGACCCAACCGCAAGGAG |
| | | GGAGCTGCGAAGGCAGGT |
| Streptococcus_ | SEQ ID | GAACGGGTGAGTAACGCGTAGGTAACCTGCCTGGTAGCGGGGGATAACTAT |
| infantis | NO: 284 | TGGAAACGATAGCTAATACCGCATAACAGTAGATATCGCATGATAGCTGCTT |
| | | GAAAGGTGCAATTGCACCACTACCAGATGGACCTGCGTTGTATTAGCTAGTT |
| | | GGTGAGGTAACGGCTCACCAAGGCRACGATACATAGCCGACCTGAGAGGGT |
| | | GATCGGCCACACTGGGACTGAGACACGGCCCAGACTCCTACGGGAGGCAGC |
| | | AGTAGGGAATCTTCGGCAATGGACGGAAGTCTGACCGAGCAACGCCGCGTG |
| | | AGTGAAGAAGGTTTTCGGATCGTAAAGCTCTGTTGTAAGAGAAGAACGAGT |
| | | GTGAGAGTGGAAAGTTCACACTGTGACGGTATCTTACCAGAAAGGGACGGC |
| | | TAACTACGTGCCAGCAGCCGCGGTAATACGTAGGTCCCGAGCGTTGTCCGG |
| | | ATTTATTGGGCGTAAAGCGAGCGCAGGCGGTTAGATAAGTCTGAAGTTAAA |
| | | GGCTGTGGCTTAACCATAGTACGCTTTGGAAACTGTTTAACTTGAGTGCAAG |
| | | AGGGGAGAGTGGAATTCCATGTGTAGCGGTGAAATGCGTAGATATATGGAG |
| | | GAACACCGGTGGCGAAAGCGGCTCTCTGGCTTGTAACTGACGCTGAGGCTC |
| | | GAAAGCGTGGGGAGCAAACAGGATTAGATACCCTGGTAGTCCACGCCGTAA |
| | | ACGATGAGTGCTAGGTGTTAGACCCTTTCCGGGGTTTAGTGCCGAAGCTAAC |
| | | GCATTAAGCACTCCGCCTGGGGAGTACGACCGCAAGGTTGAAACTCAAAGG |
| | | AATTGACGGGGGCCCGCACAAGCGGTGGAGCATGTGGTTTAATTCGAAGCA |
| | | ACGCGAAGAACCTTACCAGGTCTTGACATCCCTCTGACCGCTCTAGAGATAG |
| | | AGTTTTCCTTCGGGACAGAGGTGACAGGTGGTGCATGGTTGTCGTCAGCTCG |
| | | TGTCGTGAGATGTTGGGTTAAGTCCCGCAACGAGCGCAACCCCTATTGTTAG |
| | | TTGCCATCATTYAGTTGGGCACTCTAGCGAGACTGCCGGTAATAAACCGGAG |
| | | GAAGGTGGGGATGACGTCAAATCATCATGCCCCTTATGACCTGGGCTACAC |
| | | ACGTGCTACAATGGTTGGTACAACGAGTCGCAAGCCGGTGACGGCAAGCTA |
| | | ATCTCTTAAAGCCAATCTCAGTTCGGATTGTAGGCTGCAACTCGCCTACATG |
| | | AAGTCGGAATCGCTAGTAATCGCGGATCAGCACGCCGCGGTGAATACGTTC |
| | | CCGGGCCTTGTACACACCGCCCGTCACACCACGAGAGTTTGTAAC |
| Streptococcus_ | SEQ ID | TGCCTAATACATGCAAGTAGAACGCTGAAGGAGGAGCTTGCTCTTCTGGATG |
| infantis | NO: 285 | AGTTGCGAACGGGTGAGTAACGCGTAGGTAACCTGCCTGGTAGCGGGGGAT |
| | | AACTATTGGAAACGATAGCTAATACCGCATAACAGTAGATATCGCATGATA |
| | | GCTGCTTGAAAGGTGCAATTGCACCACTACCAGATGGACCTGCGTTGTATTA |
| | | GCTAGTTGGTGAGGTAACGGCTCACCAAGGCAACGATACATAGCCGACCTG |
| | | AGAGGGTGATCGGCCACACTGGGACTGAGACACGGCCCAGACTCCTACGGG |
| | | AGGCAGCAGTAGGGAATCTTCGGCAATGGACGGAAGTCTGACCGAGCAACG |
| | | CCGCGTGAGTGAAGAAGGTTTTCGGATCGTAAAGCTCTGTTGTAAGAGAAG |
| | | AACGAGTGTGAGAGTGGAAAGTTCACACTGTGACGGTATCTTACCAGAAAG |
| | | GGACGGCTAACTACGTGCCAGCAGCCGCGGTAATACGTAGGTCCCGAGCGT |
| | | TGTCCGGATTTATTGGGCGTAAAGCGAGCGCAGGCGGTTAGATAAGTCTGA |
| | | AGTTAAAGGCTGTGGCTTAACCATAGTACGCTTTGGAAACTGTTTAACTTGA |
| | | GTGCAAGAGGGGAGAGTGGAATTCCATGTGTAGCGGTGAAATGCGTAGATA |
| | | TATGGAGGAACACCGGTGGCGAAAGCGGCTCTCTGGCTTGTAACTGACGNG |
| | | AGGCTCGAAAGCGTGGGGAGCAAACAGGATTAGATACCCTGGTAGTCCACG |
| | | CCGTAAACGATGAGTGCTAGGTGTTAGACCCTTTCCGGGGTTTAGTGCCGTA |
| | | GCTAACGCATTAAGCACTCCGCCTGGGGAGTACGACCGCAAGGTTGAAACT |
| | | CAAAGGAATTGACGGGGGCCCGCACAAGCGGTGGAGCATGTGGTTTAATTC |
| | | GAAGCAACGCGAAGAACCTTACCAGGTCTTGACATCCCTCTGACCGCTCTAG |
| | | AGATAGAGTTTTCCTTCGGGACAGAGGTGACAGGTGGTGCATGGTTGTCGTC |
| | | AGCTCGTGTCGTGAGATGTTGGGTTAAGTCCCGCAACGAGCGCAACCCCTAT |
| | | TGTTAGTTGCCATCATTCAGTTGGGCACTCTAGCGAGACTGCCGGTAATAAA |
| | | CCGGAGGAAGGTGGGGATGACGTCAAATCATCATGCCCCTTATGACCTGGG |
| | | CTACACACGTGCTACAATGGTTGGTACAACGAGTCGCAAGCCGGTGACGGC |
| | | AAGCTAATCTCTTAAAGCCAATCTCAGTTCGGATTGTAGGCTGCAACTCGCC |
| | | TACATGAAGTCGGAATCGCTAGTAATCGCGGATCAGCACGCCGCGGTGAAT |
| | | ACG |
| Streptococcus_ | SEQ ID | GACGAACGCTGGCGGCGTGCCTAATACATGCAAGTAGAACGCTGAAGGAGG |
| infantis | NO: 286 | AGCTTGCTCTTCTGGATGAGTTGCGAACGGGTGAGTAACGCGTAGGTAACCT |
| | | GCCTGGTAGCGGGGGATAACTATTGGAAACGATAGCTAATACCGCATAACA |
| | | GTAGATATCGCATGATAGCTGCTTGAAAGGTGCAATTGCACCACTACCAGAT |
| | | GGACCTGCGTTGTATTAGCTAGTTGGTGAGGTAACGGCTCACCAAGGCAAC |
| | | GATACATAGCCGACCTGAGAGGGTGATCGGCCACACTGGGACTGAGACACG |
| | | GCCCAGACTCCTACGGGAGGCAGCAGTAGGGAATCTTCGGCAATGGACGGA |
| | | AGTCTGACCGAGCAACGCCGCGTGAGTGAAGAAGGTTTTCGGATCGTAAAG |
| | | CTCTGTTGTAAGAGAAGAACGAGTGTGAGAGTGGAAAGTTCACACTGTGAC |
| | | GGTATCTTACCAGAAAGGGACGGCTAACTACGTGCCAGCAGCCGCGGTAAT |
| | | ACGTAGGTCCCGAGCGTTGTCCGGATTTATTGGGCGTAAAGCGAGCGCAGG |
| | | CGGTTAGATAAGTCTGAAGTTAAAGGCTGTGGCTTAACCATAGTACGCTTTG |
| | | GAAACTGTTTAACTTGAGTGCAAGAGGGGAGAGTGGAATTCCATGTGTAGC |
| | | GGTGAAATGCGTAGATATATGGAGGAACACCGGTGGCGAAAGCGGCTCTCT |
| | | GGCTTGTAACTGACGCTGAGGCTCGAAAGCGTGGGGAGCAAACAGGATTAG |
| | | ATACCCTGGTAGTCCACGCCGTAAACGATGAGTGCTAGGTGTTAGACCCTTT |
| | | CCGGGGTTTAGTGCCGAAGCTAACGCATTAAGCACTCCGCCTGGGGAGTAC |
| | | GACCGCAAGGTTGAAACTCAAAGGAATTGACGGGGGCCCGCACAAGCGGTG |
| | | GAGCATGTGGTTTAATTCGAAGCAACGCGAAGAACCTTACCAGGTCTTGAC |
| | | ATCCCTCTGACCGCTCTAGAGATAGAGTTTTCCTTCGGGACAGAGGTGACAG |
| | | GTGGTGCATGGTTGTCGTCAGCTCGTGTCGTGAGATGTTGGGTTAAGTCCCG |
| | | CAACGAGCGCAACCCCTATTGTTAGTTGCCATCATTTAGTTGGGCACTCTAG |
| | | CGAGACTGCCGGTAATAAACCGGAGGAAGGTGGGGATGACGTCAAATCATC |
| | | ATGCCCCTTATGACCTGGGCTACACACGTGCTACAATGGTTGGTACAACGAG |
| | | TCGCAAGCCGGTGACGGCAAGCTAATCTCTTAAAGCCAATCTCAGTTCGGAT |
| | | TGTAGGCTGCAACTCGCCTACATGAAGTCGGAATCGCTAGTAATCGCGGATC |
| | | AGCACGCCGCGGTGAATACGTTCCCGGGCCTTGTACACACCGCCCGTCACAC |
| | | CACGAGAGTTTGTAACACCCGAAGTCGGTGAGGTAACCATTTGGAGCCAGC |
| | | CGCCTAAGGTGGGATAGATGATT |
| Clostridium_ | SEQ ID | GCGATGAAGCCCTTCGGGGTGGATTAGCGGCGGACGGGTGAGTAACACGTG |
| sp_CL_6 | NO: 287 | GGTAACCTGCCTTGTAGAGGGGGATAGCCTTCCGAAAGGAAGATTAATACC |
| | | GCATAACATCTTTTTATCGCATGGTAGAAAGATCAAAGGAGCAATCCGCTAC |
| | | AAGATGGACCCGCGGCGCATTAGCTAGTTGGTGAGGTAACGGCTCACCAAG |
| | | GCGACGATGCGTAGCCGACCTGAGAGGGTGATCGGCCACATTGGAACTGAG |
| | | ACACGGTCCAGACTCCTACGGGAGGCAGCAGTGGGGAATATTGCGCAATGG |
| | | GGGAAACCCTGACGCAGCAACGCCGCGTGAATGAAGAAGGCCTTAGGGTTG |
| | | TAAAGTTCTGTTTACGGGGACGATAATGACGGTACCCGTGGGGAAGCCACG |
| | | GCTAACTACGTGCCAGCAGCCGCGGTAATACGTAGGTGGCAAGCGTTGTCC |
| | | GGATTTACTGGGCGTAAAGGATGCGTAGGCGGATGTTTAAGTGAGATGTGA |
| | | AATACCCGGGCTCAACTTGGGTGCTGCATTTCAAACTGGACATCTAGAGTGC |
| | | GGGAGAGGAAAGCGGAATTCCTAGTGTAGCGGTGAAATGCGTAGAGATTAG |
| | | GAAGAACACCAGTGGCGAAGGCGGCTTTCTGGACCGTAACTGACGCTGAGG |
| | | CATGAAAGCGTGGGGAGCAAACAGGATTAGATACCCTGGTAGTCCACGCCG |
| | | TAAACGATGAATACTAGGTGTAGGAGGTATCGACTCCTTCTGTGCCGCAGTT |
| | | AACACAATAAGTATTCCGCCTGGGAAGTACGATCGCAAGATTAAAACTCAA |
| | | AGGAATTGACGGGGGCCCGCACAAGCAGCGGAGCATGTGGTTTAATTCGAA |
| | | GCAACGCGAAGAACCTTACCTAGACTTGACATCTCCTGAATTACTCTTAATC |
| | | GAGGAAGCCCTTCGGGGCAGGAAGACAGGTGGTGCATGGTTGTCGTCAGCT |
| | | CGTGTCGTGAGATGTTGGGTTAAGTCCCGCAACGAGCGCAACCCTTATCATT |
| | | AGTTGCTACCATTAAGTTGAGCACTCTAGTGAGACTGCCACGGTTAACGTGG |
| | | AGGAAGGTGGGGATGACGTCAAATCATCATGCCCCTTATGTCTAGGGCTAC |
| | | ACACGTGCTACAATGGTGAATACAAATAGATGCAATACCGCGAGGTGGAGC |
| | | CAAACTATAAAATTCATCCCAGTTCGGATTGCAGTCTGAAACTCGACTGCAT |
| | | GAAGCCGGAGTTGCTAGTAATCGCGAATCAGCATGTCGCGGTGAATACGTT |
| | | CCCGGGCCTTGTACACACCGCCCGTCACACCATGAGAGCTGGTAACACCCG |
| | | AAGTTCCGTGAGGTAACCGCA |

TABLE 6
Full length 16S rDNA sequences of species (positive correlates)

| | Sequence | |
|---|---|---|
| Species Name | Identifier | 16S sequence |
| Streptococcus_mitis_ | SEQ ID | aatgagagtttgatcctggctcaggacgaacgctggcggcgtgcctaatacatgcaagtagaacgctgaaggagga |
| oralis_pneumoniae | NO: 288 | gcttgcttctctggatgagagcgaacgggtgagtaacgcgtaggtaacctgcctggtagcgggggataactattgga |
| | | aacgatagctaataccgcataagagtggatgdgcatgacatttgcttaaaaggtgcacttgcatcactaccagatgga |
| | | cctgcgttgtattagctagttggtggggtaacggctcaccaaggcgacgatacatagccgacctgagagggtgatcg |
| | | gccacactgggactgagacacggcccagactcctacgggaggcagcagtagggaatcttcggcaatggacggaa |
| | | gtctgaccgagcaacgccgcgtgagtgaagaaggattcggatcgtaaagctctgttgtaagagaagaacgagtgtg |
| | | agagtggaaagttcacactgtgacggtatcttaccagaaagggacggctaactacgtgccagcagccgcggtaata |
| | | cgtaggtcccgagcgagtccggatttattgggcgtaaagcgagcgcaggcggttagataagtctgaagttaaaggct |
| | | gtggcttaaccatagtaggattggaaactgataacttgagtgcaagaggggagagtggaattccatgtgtagcggtg |
| | | aaatgcgtagatatatggaggaacaccggtggcgaaagcggctctctggcttgtaactgacgctgaggctcgaaag |
| | | cgtggggagcaaacaggattagataccctggtagtccacgctgtaaacgatgagtgctaggtgttagaccctttccgg |
| | | ggtttagtgccgtagctaacgcattaagcactccgcctggggagtacgaccgcaaggttgaaactcaaaggaattga |
| | | cgggggcccgcacaagcggtggagcatgtggtttaattcgaagcaacgcgaagaaccttaccaggtcttgacatcc |
| | | actgaccgctctagagatagagttdcatcgggacagaggtgacaggtggtgcatggttgtcgtcagctcgtgtcgt |
| | | gagatgttgggttaagtcccgcaacgagcgcaacccctattgttagttgccatcattcagttgggcactctagcgagac |
| | | tgccggtaataaaccggaggaaggtggggatgacgtcaaatcatcatgccccttatgacctgggctacacacgtgct |
| | | acaatggctggtacaacgagtcgcaagccggtgacggcaagctaatctcttaaagccagtctcagttcggattgtagg |
| | | ctgcaactcgcctacatgaagtcggaatcgctagtaatcgcggatcagcacgccgcggtgaatacgttcccgggcct |
| | | tgtacacaccgcccgtcacaccacgagagtttgtaacacccgaagtcggtgaggtaaccgtaaggagccagccgcc |
| | | taaggtgggatagatgattggggtgaagtcgtaacaaggtagccgtatcggaaggtgcggctggatcacctcctt |
| Streptococcus_mitis_ | SEQ ID | aatgagagtttgatcctggctcaggacgaacgctggcggcgtgcctaatacatgcaagtagaacgctgaaggagga |
| oralis_pneumoniae | NO: 289 | gcttgcttctctggatgagttgcgaacgggtgagtaacgcgtaggtaacctgcctggtagcgggggataactattgga |
| | | aacgatagctaataccgcataagagtggatgdgcatgacatttgataaaaggtgcacttgcatcactaccagatgga |
| | | cctgcgttgtattagctagttggtggggtaacggctcaccaaggcgacgatacatagccgacctgagagggtgatcg |
| | | gccacactgggactgagacacggcccagactcctacgggaggcagcagtagggaatcttcggcaatggacggaa |
| | | gtctgaccgagcaacgccgcgtgagtgaagaaggattcggatcgtaaagctctgttgtaagagaagaacgagtgtg |
| | | agagtggaaagttcacactgtgacggtatcttaccagaaagggacggctaactacgtgccagcagccgcggtaata |
| | | cgtaggtcccgagcgagtccggatttattgggcgtaaagcgagcgcaggcggttagataagtctgaagttaaaggct |
| | | gtggcttaaccatagtaggctttggaaactgtttaacttgagtgcaagaggggagagtggaattccatgtgtagcggtg |
| | | aaatgcgtagatatatggaggaacaccggtggcgaaagcggctctctggcttgtaactgacgctgaggctcgaaag |
| | | cgtggggagcaaacaggattagataccctggtagtccacgctgtaaacgatgagtgctaggtgttagaccctttccgg |
| | | ggtttagtgccgtagctaacgcattaagcactccgcctggggagtacgaccgcaaggttgaaactcaaaggaattga |
| | | cgggggcccgcacaagcggtggagcatgtggtttaattcgaagcaacgcgaagaaccttaccaggtcttgacatcc |
| | | ctctgaccgctctagagatagagttttccttcgggacagaggtgacaggtggtgcatggttgtcgtcagctcgtgtcgt |
| | | gagatgttgggttaagtcccgcaacgagcgcaacccctattgttagttgccatcattcagttgggcactctagcgagac |
| | | tgccggtaataaaccggaggaaggtggggatgacgtcaaatcatcatgccccttatgacctgggctacacacgtgct |
| | | acaatggctggtacaacgagtcgcaagccggtgacggcaagctaatctcttaaagccagtctcagttcggattgtagg |
| | | ctgcaactcgcctacatgaagtcggaatcgctagtaatcgcggatcagcacgccgcggtgaatacgttcccgggcct |
| | | tgtacacaccgcccgtcacaccacgagagtttgtaacacccgaagtcggtgaggtaaccgtaaggagccagccgcc |
| | | taaggtgggatagatgattggggtgaagtcgtaacaaggtagccgtatcggaaggtgcggctggatcacctcctt |
| Streptococcus_mitis_ | SEQ ID | aatgagagtttgatcctggctcaggacgaacgctggcggcgtgcctaatacatgcaagtagaacgctgaaggagga |
| oralis_pneumoniae | NO: 290 | gcttgcttctctggatgagttgcgaacgggtgagtaacgcgtaggtaacctgcctggtagcgggggataactattgga |
| | | aacgatagctaataccgcataagagtggatgdgcatgacatttgcttaaaaggtgcacttgcatcactaccagatgga |
| | | cctgcgttgtattagctagttggtggggtaacggctcaccaaggcgacgatacatagccgacctgagagggtgatcg |
| | | gccacactgggactgagacacggcccagactcctacgggaggcagcagtagggaatcttcggcaatggacggaa |
| | | gtctgaccgagcaacgccgcgtgagtgaagaaggattcggatcgtaaagctctgttgtaagagaagaacgagtgtg |
| | | agagtggaaagttcacactgtgacggtatcttaccagaaagggacggctaactacgtgccagcagccgcggtaata |
| | | cgtaggtcccgagcgagtccggatttattgggcgtaaagcgagcgcaggcggttagataagtctgaagttaaaggct |
| | | gtggcttaaccatagtaggctaggaaactgataacttgagtgcaagaggggagagtggaattccatgtgtagcggtg |
| | | aaatgcgtagatatatggaggaacaccggtggcgaaagcggctctctggcttgtaactgacgctgaggctcgaaag |
| | | cgtggggagcaaacaggattagataccctggtagtccacgctgtaaacgatgagtgctaggtgttagaccctttccgg |
| | | ggtttagtgccgtagctaacgcattaagcactccgcctggggagtacgaccgcaaggttgaaactcaaaggaattga |
| | | cgggggcccgcacaagcggtggagcatgtggtttaattcgaagcaacgcgaagaaccttaccaggtcttgacatcc |
| | | ctctgaccgctctagagatagagttttccttcgggacagaggtgacaggtggtgcatggttgtcgtcagctcgtgtcgt |
| | | gagatgttgggttaagtcccgcaacgagcgcaacccctattgttagttgccatcattcagttgggcactctagcgagac |
| | | tgccggtaataaaccggaggaaggtggggatgacgtcaaatcatcatgccccttatgacctgggctacacacgtgct |
| | | acaatggctggtacaacgagtcgcaagccggtgacggcaagctaatctcttaaagccagtctcagttcggattgtagg |
| | | ctgcaactcgcctacatgaagtcggaatcgctagtaatcgcggatcagcacgccgcggtgaatacgttcccgggcct |
| | | tgtacacaccgcccgtcacaccacgagagtttgtaacacccgaagtcggtgaggtaaccgtaaggagccagccgcc |
| | | taaggtgggatagatgattggggtgaagtcgtaacaaggtagccgtatcggaaggtgcggctggatcacctcctt |
| Streptococcus_mitis_ | SEQ ID | aatgagagtttgatcctggctcaggacgaacgctggcggcgtgcctaatacatgcaagtagaacgctgaaggagga |
| oralis_pneumoniae | NO: 291 | gcttgcttctctggatgagttgcgaacgggtgagtaacgcgtaggtaacctgcctggtagcgggggataactattgga |
| | | aacgatagctaataccgcataagagtggatgagcatgacatttgcttaaaaggtgcacttgcatcactaccagatgga |
| | | cctgcgttgtattagctagttggtggggtaacggctcaccaaggcgacgatacatagccgacctgagagggtgatcg |
| | | gccacactgggactgagacacggcccagactcctacgggaggcagcagtagggaatcttcggcaatggacggaa |
| | | gtctgaccgagcaacgccgcgtgagtgaagaaggattcggatcgtaaagactgagtaagagaagaacgagtgtg |
| | | agagtggaaagttcacactgtgacggtatcttaccagaaagggacggctaactacgtgccagcagccgcggtaata |
| | | cgtaggtcccgagcgagtccggatttattgggcgtaaagcgagcgcaggcggttagataagtctgaagttaaaggct |
| | | gtggcttaaccatagtaggctaggaaactgataacttgagtgcaagaggggagagtggaattccatgtgtagcggtg |
| | | aaatgcgtagatatatggaggaacaccggtggcgaaagcggctctctggcttgtaactgacgctgaggctcgaaag |
| | | cgtggggagcaaacaggattagataccctggtagtccacgctgtaaacgatgagtgctaggtgttagaccctttccgg |
| | | ggtttagtgccgtagctaacgcattaagcactccgcctggggagtacgaccgcaaggttgaaactcaaaggaattga |
| | | cgggggcccgcacaagcggtggagcatgtggtttaattcgaagcaacgcgaagaaccttaccaggtatgacatcc |
| | | ctctgaccgctctagagatagagttttccttcgggacagaggtgacaggtggtgcatggttgtcgtcagctcgtgtcgt |
| | | gagatgttgggttaagtcccgcaacgagcgcaacccctattgttagttgccatcattcagttgggcactctagcgagac |
| | | tgccggtaataaaccggaggaaggtggggatgacgtcaaatcatcatgccccttatgacctgggctacacacgtgct |
| | | acaatggctggtacaacgagtcgcaagccggtgacggcaagctaatctcttaaagccagtctcagttcggattgtagg |
| | | ctgcaactcgcctacatgaagtcggaatcgctagtaatcgcggatcagcacgccgcggtgaatacgttcccgggcct |
| | | tgtacacaccgcccgtcacaccacgagagtttgtaacacccgaagtcggtgaggtaaccgtaaggagccagccgcc |
| | | taaggtgggatagatgattggggtgaagtcgtaacaaggtagccgtatcggaaggtgcggctggatcacctcctt |
| Proteus_mirabilis | SEQ ID | ttgaagagatgatcatggctcagattgaacgctggcggcaggcctaacacatgcaagtcgagcggtaacaggagaa |
| | NO: 292 | agcttgattatgctgacgagcggcggacgggtgagtaatgtatggggatctgcccgatagagggggataactact |
| | | ggaaacggtggctaataccgcataatgtctacggaccaaagcaggggctatcggaccttgcactatcggatgaacc |
| | | catatgggattagctagtaggtggggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttagggagtaaagtactttcagcggggaggaaggtgataagg |
| | | ttaatacccttgtcaattgacgttacccgcagaagaagcaccggctaactccgtgccagcagccgcggtaatacgga |
| | | gggtgcaagcgttttatcggaattactgggcgtaaagcgcacgcaggcggtcaattaagtcagatgtgaaagccccg |
| | | agcttaacttgggaattgcatctgaaactggttggctagagtcttgtagaggggggtagaattccatgtgtagcggtga |
| | | aatgcgtagagatgtggaggaataccggtggcgaaggcggccccctggacaaagactgacgctcaggtgcgaaa |
| | | gcgtggggagcaaacaggattagataccctggtagtccacgctgtaaacgatgtcgatttagaggttgtggtcttgaa |
| | | ccgtggcttctggagctaacgcgttaaatcgaccgcctggggagtacggccgcaaggttaaaactcaaatgaattga |
| | | cgggggcccgcacaagcggtggagcatgtggtttaattcgatgcaacgcgaagaaccttacctactcttgacatcca |
| | | gcgaatcattagagatagaggagtgctttcgggaacgctgagacaggtgctgcatggctgtcgtcagctcgtgagt |
| | | gaaatgagggttaagtcccgcaacgagcgcaacccttatcctttgttgccagcacgtaatggtgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctgcggttggatcacctcctt |
| Proteus_mirabilis | SEQ ID | ttgaagagtttgatcatggctcagattgaacgctggcggcaggcctaacacatgcaagtcgagcggtaacaggagaa |
| | NO: 293 | agcttgctacttgctgacgagcggcggacgggtgagtaatgtatggggatctgcccgatagagggggataactact |
| | | ggaaacggtggctaataccgcataatgtctacggaccaaagcaggggctcttcggaccttgcactatcggatgaacc |
| | | catatgggattagctagtaggtggggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttagggagtaaagtactttcagcggggaggaaggtgataagg |
| | | ttaatacccttgtcaattgacgttacccgcagaagaagcaccggctaactccgtgccagcagccgcggtaatacgga |
| | | gggtgcaagcgttaatcggaattactgggcgtaaagcgcacgcaggcggtcaattaagtcagatgtgaaagccccg |
| | | agcttaacttgggaattgcatctgaaactggttggctagagtcttgtagaggggggtagaattccatgtgtagcggtga |
| | | aatgcgtagagatgtggaggaataccggtggcgaaggcggccccctggacaaagactgacgctcaggtgcgaaa |
| | | gcgtggggagcaaacaggattagataccctggtagtccacgctgtaaacgatgtcgatttagaggttgtggtcttgaa |
| | | ccgtggcttctggagctaacgcgttaaatcgaccgcctggggagtacggccgcaaggttaaaactcaaatgaattga |
| | | cgggggcccgcacaagcggtggagcatgtggtttaattcgatgcaacgcgaagaaccttacctactcttgacatcca |
| | | gcgaatcctttagagatagaggagtgccacgggaacgctgagacaggtgctgcatggctgtcgtcagctcgtgagt |
| | | gaaatgagggttaagtcccgcaacgagcgcaacccttatccatgagccagcacgtgatggtgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctttggttggatcacctcctt |
| Proteus_mirabilis | SEQ ID | ttgaagagtagatcatggctcagattgaacgctggcggcaggcctaacacatgcaagtcgagcggtaacaggagaa |
| | NO: 294 | agcttgctttcttgctgacgagcggcggacgggtgagtaatgtatggggatctgcccgatagagggggataactact |
| | | ggaaacggtggctaataccgcataatgtctacggaccaaagcaggggctcttcggaccttgcactatcggatgaacc |
| | | catatgggattagctagtaggtggggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttagggttgtaaagtactttcagcggggaggaaggtgataagg |
| | | ttaatacccttgtcaattgacgttacccgcagaagaagcaccggctaactccgtgccagcagccgcggtaatacgga |
| | | gggtgcaagcgttaatcggaattactgggcgtaaagcgcacgcaggcggtcaattaagtcagatgtgaaagccccg |
| | | agcttaacttgggaattgcatctgaaactggttggctagagtatgtagaggggggtagaattccatgtgtagcggtga |
| | | aatgcgtagagatgtggaggaataccggtggcgaaggcggccccctggacaaagactgacgctcaggtgcgaaa |
| | | gcgtggggagcaaacaggattagataccctggtagtccacgctgtaaacgatgtcgatttagaggttgtggtcttgaa |
| | | ccgtggcttctggagctaacgcgttaaatcgaccgcctggggagtacggccgcaaggttaaaactcaaatgaattga |
| | | cgggggcccgcacaagcggtggagcatgtggtttaattcgatgcaacgcgaagaaccttacctactcttgacatcca |
| | | gcgaatccatagagatagaggagtgccacgggaacgctgagacaggtgctgcatggctgtcgtcagctcgtgttgt |
| | | gaaatgagggttaagtcccgcaacgagcgcaacccttatcctttgttgccagcacgtaatggtgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctgcggttggatcacctcctt |
| Proteus_mirabilis | SEQ ID | ttgaagagtttgatcatggctcagattgaacgctggcggcaggcctaacacatgcaagtcgagcggtaacaggagaa |
| | NO: 295 | agcttgctacttgctgacgagcggcggacgggtgagtaatgtatggggatctgcccgatagagggggataactact |
| | | ggaaacggtggctaataccgcataatgtctacggaccaaagcaggggctcttcggaccttgcactatcggatgaacc |
| | | catatgggattagctagtaggtggggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttagggagtaaagtactttcagcggggaggaaggtgataagg |
| | | ttaatacccttatcaattgacgttacccgcagaagaagcaccggctaactccgtgccagcagccgcggtaatacgga |
| | | gggtgcaagcgttaatcggaattactgggcgtaaagcgcacgcaggcggtcaattaagtcagatgtgaaagccccg |
| | | agcttaacttgggaattgcatctgaaactggttggctagagtcttgtagaggggggtagaattccatgtgtagcggtga |
| | | aatgcgtagagatgtggaggaataccggtggcgaaggcggccccctggacaaagactgacgctcaggtgcgaaa |
| | | gcgtggggagcaaacaggattagataccctggtagtccacgctgtaaacgatgtcgatttagaggttgtggtcttgaa |
| | | ccgtggcttctggagctaacgcgttaaatcgaccgcctggggagtacggccgcaaggttaaaactcaaatgaattga |
| | | cgggggcccgcacaagcggtggagcatgtggtttaattcgatgcaacgcgaagaaccttacctactcttgacatcca |
| | | gcgaatcctttagagatagaggagtgccttcgggaacgctgagacaggtgctgcatggctgtcgtcagctcgtgagt |
| | | gaaatgttgggttaagtcccgcaacgagcgcaacccttatcctttgttgccagcacgtaatggtgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctgcggttggatcacctcctt |
| Proteus_mirabilis | SEQ ID | ttgaagagtttgatcatggctcagattgaacgctggcggcaggcctaacacatgcaagtcgagcggtaacaggagaa |
| | NO: 296 | agcttgctttcttgctgacgagcggcggacgggtgagtaatgtatggggatctgcccgatagagggggataactact |
| | | ggaaacggtggctaataccgcataatgtctacggaccaaagcaggggctcttcggaccttgcactatcggatgaacc |
| | | catatgggattagctagtaggtggggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttaggogtaaagtactttcagcggggaggaaggtgataagg |
| | | ttaatacccttgtcaattgacgttacccgcagaagaagcaccggctaactccgtgccagcagccgcggtaatacgga |
| | | gggtgcaagcgttaatcggaattactgggcgtaaagcgcacgcaggcggtcaattaagtcagatgtgaaagccccg |
| | | agcttaacttgggaattgcatctgaaactggttggctagagtcttgtagaggggggtagaattccatgtgtagcggtga |
| | | aatgcgtagagatgtggaggaataccggtggcgaaggcggccccctggacaaagactgacgctcaggtgcgaaa |
| | | gcgtggggagcaaacaggattagataccctggtagtccacgctgtaaacgatgtcgatttagaggttgtggtcttgaa |
| | | ccgtggcttctggagctaacgcgttaaatcgaccgcctggggagtacggccgcaaggttaaaactcaaatgaattga |
| | | cgggggcccgcacaagcggtggagcatgtggtttaattcgatgcaacgcgaagaaccttacctactcttgacatcca |
| | | gcgaatcctttagagatagaggagtgccttcgggaacgctgagacaggtgctgcatggctgtcgtcagctcgtgttgt |
| | | gaaatgttgggttaagtcccgcaacgagcgcaacccttatcctttgttgccagcacgtgatggtgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctgcggttggatcacctcctt |
| Proteus_mirabilis | SEQ ID | ttgaagagtttgatcatggctcagattgaacgctggcggcaggcctaacacatgcaagtcgagcggtaacaggagaa |
| | NO: 297 | agcttgctttcttgctgacgagcggcggacgggtgagtaatgtatggggatctgcccgatagagggggataactact |
| | | ggaaacggtggctaataccgcataatgtctacggaccaaagcaggggctcttcggaccttgcactatcggatgaacc |
| | | catatgggattagctagtaggtggggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttagggttgtaaagtactttcagcggggaggaaggtgataagg |
| | | ttaatacccttatcaattgacgttacccgcagaagaagcaccggctaactccgtgccagcagccgcggtaatacgga |
| | | gggtgcaagcgttaatcggaattactgggcgtaaagcgcacgcaggcggtcaattaagtcagatgtgaaagccccg |
| | | agcttaacttgggaattgcatctgaaactggttggctagagtcttgtagaggggggtagaattccatgtgtagcggtga |
| | | aatgcgtagagatgtggaggaataccggtggcgaaggcggccccctggacaaagactgacgctcaggtgcgaaa |
| | | gcgtggggagcaaacaggattagataccctggtagtccacgctgtaaacgatgtcgatttagaggttgtggtcttgaa |
| | | ccgtggcttctggagctaacgcgttaaatcgaccgcctggggagtacggccgcaaggttaaaactcaaatgaattga |
| | | cgggggcccgcacaagcggtggagcatgtggtttaattcgatgcaacgcgaagaaccttacctactcttgacatcca |
| | | gcgaatcctttagagatagaggagtgccttctggaacgctgagacaggtgctgcatggctgtcgtcagctcgtgttgt |
| | | gaaatgttgggttaagtcccgcaacgagcgcaacccttatcctttgttgccagcacgtaatggtgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctgcggttggatcacctcctt |
| Proteus_mirabilis | SEQ ID | ttgaagagtagatcatggctcagattgaacgctggcggcaggcctaacacatgcaagtcgagcggtaacaggagaa |
| | NO: 298 | agcttgctacttgctgacgagcggcggacgggtgagtaatgtatggggatctgcccgatagagggggataactact |
| | | ggaaacggtggctaataccgcataatgtctacggaccaaagcaggggctcttcggaccttgcactatcggatgaacc |
| | | catatgggattagctagtaggtggggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttagggagtaaagtactttcagcggggaggaaggtgataagg |
| | | ttaatacccttatcaattgacgttacccgcagaagaagcaccggctaactccgtgccagcagccgcggtaatacgga |
| | | gggtgcaagcgttaatcggaattactgggcgtaaagcgcacgcaggcggtcaattaagtcagatgtgaaagccccg |
| | | agcttaacttgggaattgcatctgaaactggttggctagagtatgtagaggggggtagaattccatgtgtagcggtga |
| | | aatgcgtagagatgtggaggaataccggtggcgaaggcggccccctggacaaagactgacgctcaggtgcgaaa |
| | | gcgtggggagcaaacaggattagataccctggtagtccacgctgtaaacgatgtcgatttagaggttgtggtcttgaa |
| | | ccgtggcttctggagctaacgcgttaaatcgaccgcctggggagtacggccgcaaggttaaaactcaaatgaattga |
| | | cgggggcccgcacaagcggtggagcatgtggtttaattcgatgcaacgcgaagaaccttacctactcttgacatcca |
| | | gcgaatcctttagagatagaggagtgccttctggaacgctgagacaggtgctgcatggctgtcgtcagctcgtgttgt |
| | | gaaatgttgggttaagtcccgcaacgagcgcaacccttatccatgagccagcacgtaatggtgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctgcggttggatcacctcctt |
| Pediococcus_ | SEQ ID | atgagagtttgatcttggctcaggatgaacgctggcggcgtgcctaatacatgcaagtcgaacgaacttccgttaattg |
| acidilactici | NO: 299 | atcaggacgtgcttgcactgaatgagattttaacacgaagtgagtggcggacgggtgagtaacacgtgggtaacctg |
| | | cccagaagcaggggataacacctggaaacagatgctaataccgtataacagagaaaaccgcctggtatcttttaaaa |
| | | gatggctctgctatcacttctggatggacccgcggcgcattagctagttggtgaggtaacggctcaccaaggcgatga |
| | | tgcgtagccgacctgagagggtaatcggccacattgggactgagacacggcccagactcctacgggaggcagca |
| | | gtagggaatcttccacaatggacgcaagtctgatggagcaacgccgcgtgagtgaagaagggtttcggctcgtaaa |
| | | gctctgttgttaaagaagaacgtgggtgagagtaactgttcacccagtgacggtatttaaccagaaagccacggctaa |
| | | ctacgtgccagcagccgcggtaatacgtaggtggcaagcgttatccggatttattgggcgtaaagcgagcgcaggc |
| | | ggtatttaagtctaatgtgaaagcatcggctcaaccgaagaagtgcattggaaactgggagacttgagtgcagaag |
| | | aggacagtggaactccatgtgtagcggtgaaatgcgtagatatatggaagaacaccagtggcgaaggcggctgtct |
| | | ggtctgtaactgacgctgaggctcgaaagcatgggtagcgaacaggattagataccctggtagtccatgccgtaaac |
| | | gatgattactaagtgaggagggatccgcccttcagtgctgcagctaacgcattaagtaatccgcctggggagtacga |
| | | ccgcaaggttgaaactcaaaagaattgacgggggcccgcacaagcggtggagcatgtggtttaattcgaagctacg |
| | | cgaagaaccttaccaggtcttgacatcttctgccaacctaagagattaggcgttcccttcggggacagaatgacaggt |
| | | ggtgcatggttgtcgtcagctcgtgtcgtgagatgttgggttaagtcccgcaacgagcgcaacccttattactagttgcc |
| | | agcattcagttgggcactctagtgagactgccggtgacaaaccggaggaaggtggggacgacgtcaaatcatcatg |
| | | ccccttatgacctgggctacacacgtgctacaatggatggtacaacgagttgcgaaaccgcgaggtttagctaatctct |
| | | taaaaccattctcagttcggactgtaggctgcaactcgcctacacgaagtcggaatcgctagtaatcgcggatcagca |
| | | tgccgcggtgaatacgttcccgggccttgtacacaccgcccgtcacaccatgagagtttgtaacacccaaagccggt |
| | | ggggtaaccttttaggagctagccgtctaaggtgggacagatgattagggtgaagtcgtaacaaggtagccgtagga |
| | | gaacctgcggctggatcacctcctt |
| Bifidobacterium_ | SEQ ID | tatggagggttcgattctggctcaggatgaacgctggcggcgtgcttaacacatgcaagtcgaacgggatcccggg |
| dendum | NO: 300 | ggttcgctcccgggtgagagtggcgaacgggtgagtaatgcgtgaccgacctgccccatacaccggaatagctcct |
| | | ggaaacgggtggtaatgccggatgctccggttggatgcatgtccttccgggaaagattccatcggtatgggatgggg |
| | | tcgcgtcctatcagcttgatggcggggtaacggcccaccatggcttcgacgggtagccggcctgagagggcgacc |
| | | ggccacattgggactgagatacggcccagactcctacgggaggcagcagtggggaatattgcacaatgggcgcaa |
| | | gcctgatgcagcgacgccgcgtgcgggatggaggccttcgggagtaaaccgctatgatcgggagcaagcccttc |
| | | ggggtgagtgtacctttcgaataagcaccggctaactacgtgccagcagccgcggtaatacgtagggtgcaagcgtt |
| | | atccggaattattgggcgtaaagggctcgtaggcggttcgtcgcgtccggtgtgaaagcccatcgcttaacggtgggt |
| | | ctgcgccgggtacgggcgggctggagtgcggtaggggagactggaattcccggtgtaacggtggaatgtgtagat |
| | | atcgggaagaacaccaatggcgaaggcaggtctctgggccgtcactgacgctgaggagcgaaagcgtggggagc |
| | | gaacaggattagataccctggtagtccacgccgtaaacggtggatgctggatgtggggcccgttccacgggttccgt |
| | | gtcggagctaacgcgttaagcatcccgcctggggagtacggccgcaaggctaaaactcaaagaaattgacggggg |
| | | cccgcacaagcggcggagcatgcggattaattcgatgcaacgcgaagaaccttacctgggcttgacatgacccgac |
| | | agccgtagagatatggcctcccttcggggcgggttcacaggtggtgcatggtcgtcgtcagctcgtgtcgtgagatgt |
| | | tgggttaagtcccgcaacgagcgcaaccctcgccctgtgagccagcacgtcatggtgggaactcacgggggaccg |
| | | ccggggtcaactcggaggaaggtggggatgacgtcagatcatcatgccccttacgtccagggcttcacgcatgctac |
| | | aatggccggtacagcgggatgcgacatggcgacatggagcggatccctgaaaaccggtctcagttcggattggagt |
| | | ctgcaacccgactccatgaaggcggagtcgctagtaatcgcggatcagcaacgccgcggtgaatgcgttcccggg |
| | | ccagtacacaccgcccgtcaagtcatgaaagtgggcagcacccgaagccggtggcctaaccccttgcgggatgga |
| | | gccgtctaaggtgaggctcgtgattgggactaagtcgtaacaaggtagccgtaccggaaggtgcggctggatcacct |
| | | ccttt |
| Proteus_penneri | SEQ ID | ttgaagagtttgatcatggctcagattgaacgctggcggcaggcctaacacatgcaagtcgagcggtaacagaagaa |
| | NO: 301 | agcttgctacttgctgacgagcggcggacgggtgagtaatgtatggggatctgcccgatagagggggagtaactac |
| | | tggaaacggtggctaataccgcatgacgtctacggaccaaagcaggggctcttcggaccttgcgctatcggatgaac |
| | | ccatatgggattagctagtaggtgaggtaaaggctcacctaggcgacgatctctagctggtctgagaggatgatcagc |
| | | cacactgggactgagacacggcccagactcctacgggaggcagcagtggggaatattgcacaatgggcgcaagc |
| | | ctgatgcagccatgccgcgtgtatgaagaaggccttagggagtaaagtactttcagcggggaggaaggtgataaag |
| | | ttaatacctttatcaattgacgttacccgcagaagaagcaccggctaactccgtgccagcagccgcggtaatacggag |
| | | ggtgcaagcgttaatcggaattactgggcgtaaagcgcacgcaggcggtcaattaagtcagatgtgaaagccccga |
| | | gcttaacttgggaattgcatctgaaactggttggctagagtcagtagaggggggtagaattccatctgtagcggtgaa |
| | | atgcgtagagatgtggaggaataccggtggcgaaggcggccccctggacaaagactgacgctcaggtgcgaaag |
| | | cgtggggagcaaacaggattagataccctggtagtccacgctgtaaacgatgtcgatttagaggttgtggtcttgaac |
| | | cgtggcttctggagctaacgcgttaaatcgaccgcctggggagtacggccgcaaggttaaaactcaaatgaattgac |
| | | gggggcccgcacaagcggtggagcatgtggtttaattcgatgcaacgcgaagaaccttacctactcttgacatccag |
| | | cggatcctttagagatagaggagtgccttcgggaacgctgagacaggtgctgcatggctgtcgtcagctcgtgttgtg |
| | | aaatgttgggttaagtcccgcaacgagcgcaacccttatcctttgttgccagcgcgtgatggcgggaactcaaagga |
| | | gactgccggtgataaaccggaggaaggtggggatgacgtcaagtcatcatggcccttacgagtagggctacacacg |
| | | tgctacaatggcagatacaaagagaagcgacctcgcgagagcaagcggaactcataaagtctgtcgtagtccggatt |
| | | ggagtctgcaactcgactccatgaagtcggaatcgctagtaatcgtagatcagaatgctacggtgaatacgttcccgg |
| | | gccttgtacacaccgcccgtcacaccatgggagtgggttgcaaaagaagtaggtagcttaaccttcgggagggcgct |
| | | taccactttgtgattcatgactggggtgaagtcgtaacaaggtaaccgtaggggaacctgcggttggatcacctcctt |
| Proteus_penneri | SEQ ID | ttgaagagtttgatcatggctcagattgaacgctggcggcaggcctaacgcatgcaagtcgaccggaaacagagga |
| | NO: 302 | aagcttgctttcttgctgacgagcggcggacgggtgagtaatgtatggggatctgcccgatagaggggggtaactatt |
| | | ggaaaaagggggggtaaataccgcttgacgtctacggccaaaagccggggcgcttcggacctttcgctattggatg |
| | | aacccatatgggattagctagtaggtgaggtaaaggctcacctaggcgacgatctctagctggtctgagaggatgatc |
| | | agccacactgggactgagacacggcccagactcctacgggaggcagcagtggggaatattgcacaatgggcgca |
| | | agcctgatgcagccatgccgcgtgtatgaagaaggccttagggttgtaaagtactttcagcggggaggaaggtgata |
| | | aagttaatacctttatcaattgacgttacccgcagaagaagcaccggctaactccgtgccagcagccgcggtaatacg |
| | | gagggtgcaagcgttaatcggaattactgggcgtaaagcgcacgcaggcggtcaattaagtcagatgtgaaagccc |
| | | cgagcttaacttgggaattgcatctgaaactggttggctagagtcagtagagggggggtagaattccatgtgtagcgg |
| | | tgaaatgcgtagagatgtggaggaataccggtggcgaaggcggccccctggacaaagactgacgctcaggtgcga |
| | | aagcgtggggagcaaacaggattagataccctggtagtccacgctgtaaacgatgtcgatttagaggttgtggtcttg |
| | | aaccgtggcttctggagctaacgcgttaaatcgaccgcctggggagtacggccgcaaggttaaaactcaaatgaatt |
| | | gacgggggcccgcacaagcggtggagcatgtggtttaattcgatgcaacgcgaagaaccttacctactcttgacatc |
| | | cagcgaatcctttagagatagaggagtgccttcgggaacgctgagacaggtgctgcatggctgtcgtcagctcgtgtt |
| | | gtgaaatgagggttaagtcccgcaacgagcgcaacccttatcctagttgccagcgcgtgatggcgggaactcaaag |
| | | gagactgccggtgataaaccggaggaaggtggggatgacgtcaagtcatcatggcccttacgagtagggctacaca |
| | | cgtgctacaatggcagatacaaagagaagcgacctcgcgagagcaagcggaactcataaagtctgtcgtagtccgg |
| | | attggagtctgcaactcgactccatgaagtcggaatcgctagtaatcgtagatcagaatgctacggtgaatacg |
| Lactobacillus_ | SEQ ID | atgagagtagatcctggctcaggacgaacgctggcggcgtgcctaatacatgcaagtcgaacgaaactttcttacac |
| salivarius | NO: 303 | cgaatgcttgcattcatcgtaagaagttgagtggcggacgggtgagtaacacgtgggtaacctgcctaaaagaaggg |
| | | gataacacttggaaacaggtgctaataccgtatatctctaaggatcgcatgatccttagatgaaagatggttctgctatc |
| | | gcttttagatggacccgcggcgtattaactagttggtggggtaacggcctaccaaggtgatgatacgtagccgaactg |
| | | agaggttgatcggccacattgggactgagacacggcccaaactcctacgggaggcagcagtagggaatcttccaca |
| | | atggacgcaagtctgatggagcaacgccgcgtgagtgaagaaggtcttcggatcgtaaaactctgttgttagagaag |
| | | aacacgagtgagagtaactgttcattcgatgacggtatctaaccagcaagtcacggctaactacgtgccagcagccg |
| | | cggtaatacgtaggtggcaagcgagtccggatttattgggcgtaaagggaacgcaggcggtctataagtctgatgtg |
| | | aaagccttcggcttaaccggagtagtgcattggaaactggaagacttgagtgcagaagaggagagtggaactccat |
| | | gtgtagcggtgaaatgcgtagatatatggaagaacaccagtggcgaaagcggctctctggtctgtaactgacgctga |
| | | ggttcgaaagcgtgggtagcaaacaggattagataccctggtagtccacgccgtaaacgatgaatgctaggtgagg |
| | | agggtttccgcccttcagtgccgcagctaacgcaataagcattccgcctggggagtacgaccgcaaggttgaaactc |
| | | aaaggaattgacgggggcccgcacaagcggtggagcatgtggtttaattcgaagcaacgcgaagaaccttaccag |
| | | gtcttgacatccatgaccacctaagagattaggtttt |
| Veillonella_dispar | SEQ ID | ttggagagtttgatcctggctcaggacgaacgctggcggcgtgcttaacacatgcaagtcgaacgaagagcgatgg |
| | NO: 304 | aagcttgcttctatcaatcttagtggcgaacgggtgagtaacgcgtaatcaacctgcccttcagagggggacaacagt |
| | | tggaaacgactgctaataccgcatacgatctaacctcggcatcgaggatagatgaaaggtggcctctatttataagcta |
| | | tcactgaaggaggggattgcgtctgattagctagttggaggggtaacggcccaccaaggcgatgatcagtagccgg |
| | | tctgagaggatgaacggccacattgggactgagacacggcccagactcctacgggaggcagcagtggggaatctt |
| | | ccgcaatggacgaaagtctgacggagcaacgccgcgtgagtgatgacggccttcgggttgtaaagctctgttaatcg |
| | | ggacgaaaggccttcttgcgaatagttagaaggattgacggtaccggaatagaaagccacggctaactacgtgcca |
| | | gcagccgcggtaatacgtaggtggcaagcgttgtccggaattattgggcgtaaagcgcgcgcaggcggattggtca |
| | | gtctgtcttaaaagttcggggcttaaccccgtgatgggatggaaactgccaatctagagtatcggagaggaaagtgga |
| | | attcctagtgtagcggtgaaatgcgtagatattaggaagaacaccagtggcgaaggcgactttctggacgaaaactg |
| | | acgctgaggcgcgaaagccaggggagcgaacgggattagataccccggtagtcctggccgtaaacgatgggtact |
| | | aggtgtaggaggtatcgaccccactgtgccggagttaacgcaataagtaccccgcctggggagtacgaccgcaag |
| | | gttgaaactcaaaggaattgacgggggcccgcacaagcggtggagtatgtggtttaattcgacgcaacgcgaagaa |
| | | ccttaccaggtcttgacattgatggacagaactagagatagttcctcdcttcggaagccagaaaacaggtggtgcacg |
| | | gttgtcgtcagctcgtgtcgtgagatgagggttaagtcccgcaacgagcgcaacccctatcttatgagccagcactd |
| | | gggtgggaactcatgagagactgccgcagacaatgcggaggaaggcggggatgacgtcaaatcatcatgcccctt |
| | | atgacctgggctacacacgtactacaatgggagttaatagacggaagcaataccgcgaggtggagcaaacccgag |
| | | aaacactctctcagttcggatcgtaggctgcaactcgcctacgtgaagtcggaatcgctagtaatcgcaggtcagcat |
| | | actgcggtgaatacgttcccgggccagtacacaccgcccgtcacaccacgaaagtcggaagtgcccaaagccggt |
| | | ggggtaaccttcgggagccagccgtctaaggtaaagtcgatgattggggtgaagtcgtaacaaggtagccgtatcg |
| | | gaaggtgcggctggattacctccttt |
| Veillonella_dispar | SEQ ID | ttggagagtttgatcctggctcaggacgaacgctggcggcgtgcttaacacatgcaagtcgaacgaagagcgatgg |
| | NO: 305 | aagcttgcttctatcaatcttagtggcgaacgggtgagtaacgcgtaatcaacctgcccttcagagggggacaacagt |
| | | tggaaacgactgctaataccgcatacgatccaatctcggcatcgaggatagatgaaaggtggcctctatttataagcta |
| | | tcactgaaggaggggattgcgtctgattagctagttggaggggtaacggcccaccaaggcgatgatcagtagccgg |
| | | tctgagaggatgaacggccacattgggactgagacacggcccagactcctacgggaggcagcagtggggaatctt |
| | | ccgcaatggacgaaagtctgacggagcaacgccgcgtgagtgatgacggccttcgggttgtaaagctctgttaatcg |
| | | ggacgaaaggccttcttgcgaatagttagaaggattgacggtaccggaatagaaagccacggctaactacgtgcca |
| | | gcagccgcggtaatacgtaggtggcaagcgttgtccggaattattgggcgtaaagcgcgcgcaggcggattggtca |
| | | gtctgtcttaaaagttcggggcttaaccccgtgatgggatggaaactgccaatctagagtatcggagaggaaagtgga |
| | | attcctagtgtagcggtgaaatgcgtagatattaggaagaacaccagtggcgaaggcgactttctggacgaaaactg |
| | | acgctgaggcgcgaaagccaggggagcgaacgggattagataccccggtagtcctggccgtaaacgatgggtact |
| | | aggtgtaggaggtatcgaccccttctgtgccggagttaacgcaataagtaccccgcctggggagtacgaccgcaag |
| | | gttgaaactcaaaggaattgacgggggcccgcacaagcggtggagtatgtggtttaattcgacgcaacgcgaagaa |
| | | ccttaccaggtcttgacattgatggacagaactagagatagttcctcttcttcggaagccagaaaacaggtggtgcacg |
| | | aggttgtcgtcagctcgtgtcgtgatgagggttaagtcccgcaacgagcgcaacccctatcttatgagccagcactd |
| | | gggtgggaactcatgagagactgccgcagacaatgcggaggaaggcggggatgacgtcaaatcatcatgcccctt |
| | | atgacctgggctacacacgtactacaatgggagttaatagacggaagcaataccgcgaggtggagcaaacccgag |
| | | aaacactctctcagttcggatcgtaggctgcaactcgcctacgtgaagtcggaatcgctagtaatcgcaggtcagcat |
| | | actgcggtgaatacgttcccgggccagtacacaccgcccgtcacaccacgaaagtcggaagtgcccaaagccggt |
| | | ggggtaaccttcgggagccagccgtctaaggtaaagtcgatgattggggtgaagtcgtaacaaggtagccgtatcg |
| | | gaaggtgcggctggatcacctccat |
| Veillonella_dispar | SEQ ID | ttggagagtttgatcctggctcaggacgaacgctggcggcgtgcttaacacatgcaagtcgaacgaagagcgatgg |
| | NO: 306 | aagcttgcactatcaatcttagtggcgaacgggtgagtaacgcgtaatcaacctgcccttcagagggggacaacagt |
| | | tggaaacgactgctaataccgcatacgatctaacctcggcatcgaggatagatgaaaggtggcctctatttataagcta |
| | | tcactgaaggaggggattgcgtctgattagctagttggaggggtaacggcccaccaaggcgatgatcagtagccgg |
| | | tctgagaggatgaacggccacattgggactgagacacggcccagactcctacgggaggcagcagtggggaatat |
| | | ccgcaatggacgaaagtctgacggagcaacgccgcgtgagtgatgacggccttcgggttgtaaagctctgttaatcg |
| | | ggacgaaaggccacttgcgaatagttagaaggattgacggtaccggaatagaaagccacggctaactacgtgcca |
| | | gcagccgcggtaatacgtaggtggcaagcgttgtccggaattattgggcgtaaagcgcgcgcaggcggattggtca |
| | | gtctgtcttaaaagttcggggcttaaccccgtgatgggatggaaactgccaatctagagtatcggagaggaaagtgga |
| | | attcctagtgtagcggtgaaatgcgtagatattaggaagaacaccagtggcgaaggcgactttctggacgaaaactg |
| | | aacgctgaggcgcgaagccaggggagcgaacgggattagataccccggtagtcctggccgtaaacgatgggtact |
| | | aggtgtaggaggtatcgaccccactgtgccggagttaacgcaataagtaccccgcctggggagtacgaccgcaag |
| | | gagaaactcaaaggaattgacgggggcccgcacaagcggtggagtatgtggtaaattcgacgcaacgcgaagaa |
| | | atggacagccttaccaggtcttgacattgaactagagatagacctcttcttcggaagccagaaaacaggtggtgcacg |
| | | gagtcgtcagctcgtgtcgtgagatgagggttaagtcccgcaacgagcgcaacccctatcttatgagccagcacttt |
| | | gggtgggaactcatgagagactgccgcagacaatgcggaggaaggcggggatgacgtcaaatcatcatgcccctt |
| | | atgacctgggctacacacgtactacaatgggagttaatagacggaagcaataccgcgaggtggagcaaacccgag |
| | | aaacactctctcagttcggatcgtaggctgcaactcgcctacgtgaagtcggaatcgctagtaatcgcaggtcagcat |
| | | actgcggtgaatacgttcccgggccagtacacaccgcccgtcacaccacgaaagtcggaagtgcccaaagccggt |
| | | ggggtaaccttcgggagccagccgtctaaggtaaagtcgatgattggggtgaagtcgtaacaaggtagccgtatcg |
| | | gaaggtgcggctggatcacctccat |
| Rothia_dentocariosa | SEQ ID | acggagagtttgattctggctcaggacgaacgctggcggcgtgcttaacacatgcaagtcgaacgatgaagcctagc |
| | NO: 307 | ttgctaggtggattagtggcgaacgggtgagtaatacgtgagtgacctacctttgactctgggataagcctgggaaact |
| | | aatctccgcgggtctaataccggatacgaccatggggtgttggtggaaagcgttatggagtggttttagatgggctca |
| | | cggcctatcagatgaggtgaggtaatggcttaccaaggcgacgacgggtagccggcctgagagggtgaccggcc |
| | | acactgggactgagacacggcccagactcctacgggaggcagcagtggggaatattgcacaatgggcgcaagcct |
| | | gatgcagcgacgccgcgtgagggatgacggccttcgggttgtaaacctctgttagcatcgaagaagcgaaagtgac |
| | | ggtaggtgcagagaaagcgccggctaactacgtgccagcagccgcggtaatacgtagggcgcgagcgttgtccg |
| | | gaattattgggcgtaaagagcttgtaggcggttggtcgcgtctgctgtgaaaggctggggcttaaccctggtatgcag |
| | | tgggtacgggctaactagagtgcagtaggggagactggaattcctggtgtagcggtggaatgcgcagatatcagga |
| | | ggaacaccgatggcgaaggcaggtctctgggctgtaactgacgctgagaagcgaaagcatggggagcgaacagg |
| | | attagataccctggtagtccatgccgtaaacgagggcactaggtgtgggggacattccacgtatccgcgccgtagct |
| | | aacgcattaagtgccccgcctggggagtacggccgcaaggctaaaactcaaagaaattgacgggggcccgcacaa |
| | | gcggcggagcatgcggattaattcgatgcaacgcgaagaaccttaccaaggcttgacatatactggactgcgtcaga |
| | | gatggcgdtccatcggggctggtatacaggtggtgcatggttgtcgtcagctcgtgtcgtgagatgagggttaagtc |
| | | ccgcaacgagcgcaaccctcgttctatgttgccagcacgtgatggtggggactcataggagactgccggggtcaact |
| | | cggaggaaggtggggatgacgtcaaatcatcatgcccatatgtcagggcttcacgcatgctacaatggccggtaca |
| | | gagggttgcgatactgtgaggtggagctaatccctaaaagccggtctcagttcggattggggtctgcaactcgacccc |
| | | atgaagtcggagtcgctagtaatcgcagatcagcaacgctgcggtgaatacgttcccgggccttgtacacaccgccc |
| | | gtcaagtcacgaaagttggtaacacccgaagccggtggcctaaccctggtggggggagccgtcgaaggtgggact |
| | | ggcgattgggactaagtcgtaacaaggtagccgtaccggaaggtgcggctggatcacctcatt |
| Rothia_dentocariosa | SEQ ID | acggagagtttgattctggctcaggacgaacgctggcggcgtgcttaacacatgcaagtcgaacgatgaagcctagc |
| | NO: 308 | ttgctaggtggattagtggcgaacgggtgagtaatacgtgagtgacctacctttgactctgggataagcctgggaaact |
| | | aatctccgcgggtctaataccggatacgaccatggggtgttggtggaaagcgttatggagtggttttagatgggctca |
| | | cggcctatcagcttgaggtgaggtaatggcttaccaaggcgacgacgggtagccggcctgagagggtgaccggcc |
| | | acactgggactgagacacggcccagactcctacgggaggcagcagtggggaatattgcacaatgggcgcaagcct |
| | | gatgcagcgacgccgcgtgagggatgacggccttcgggttgtaaacctctgttagcatcgaagaagcgaaagtgac |
| | | ggtaggtgcagagaaagcgccggctaactacgtgccagcagccgcggtaatacgtagggcgcgagcgttgtccg |
| | | gaattattgggcgtaaagagcttgtaggcggttggtcgcgtctgctgtgaaaggctggggcttaaccctggtatgcag |
| | | tgggtacgggctaactagagtgcagtaggggagactggaattcctggtgtagcggtggaatgcgcagatatcagga |
| | | ggaacaccgatggcgaaggcaggtctctgggctgtaactgacgctgagaagcgaaagcatggggagcgaacagg |
| | | attagataccctggtagtccatgccgtaaacgttgggcactaggtgtgggggacattccacgtatccgcgccgtagct |
| | | aacgcattaagtgccccgcctggggagtacggccgcaaggctaaaactcaaagaaattgacgggggcccgcacaa |
| | | gcggcggagcatgcggattaattcgatgcaacgcgaagaaccttaccaaggcttgacatatactggactgcgtcaga |
| | | gatggcgdtccatcggggctggtatacaggtggtgcatggttgtcgtcagctcgtgtcgtgagatgttgggttaagtc |
| | | ccgcaacgagcgcaaccctcgttctatgttgccagcacgtgatggtggggactcataggagactgccggggtcaact |
| | | cggaggaaggtggggatgacgtcaaatcatcatgccccttatgtcagggatcacgcatgctacaatggccggtaca |
| | | gagggttgcgatactgtgaggtggagctaatccctaaaagccggtctcagttcggattggggtctgcaactcgacccc |
| | | atgaagtcggagtcgctagtaatcgcagatcagcaacgctgcggtgaatacgttcccgggccttgtacacaccgccc |
| | | gtcaagtcacgaaagttggtaacacccgaagccggtggcctaaccctggtggggggagccgtcgaaggtgggact |
| | | ggcgattgggactaagtcgtaacaaggtagccgtaccggaaggtgcggctggatcacctccttt |
| Rothia_dentocariosa | SEQ ID | acggagagtttgattctggctcaggacgaacgctggcggcgtgcttaacacatgcaagtcgaacgatgaagcctagc |
| | NO: 309 | ttgctaggtggattagtggcgaacgggtgagtaatacgtgagtgacctacctttgactctgggataagcctgggaaact |
| | | gggtctaataccggatacgaccaatctccgcatggggtgttggtggaaagcgttatggagtggttttagatgggctca |
| | | cggcctatcagcttgttggtgaggtaatggcttaccaaggcgacgacgggtagccggcctgagagggtgaccggcc |
| | | acactgggactgagacacggcccagactcctacgggaggcagcagtggggaatattgcacaatgggcgcaagcct |
| | | gatgcagcgacgccgcgtgagggatgacggccttcgggttgtaaacctctgttagcatcgaagaagcgaaagtgac |
| | | ggtaggtgcagagaaagcgccggctaactacgtgccagcagccgcggtaatacgtagggcgcgagcgttgtccg |
| | | gaattattgggcgtaaagagcttgtaggcggttggtcgcgtctgctgtgaaaggctggggcttaaccctggtatgcag |
| | | tgggtacgggctaactagagtgcagtaggggagactggaattcctggtgtagcggtggaatgcgcagatatcagga |
| | | ggaacaccgatggcgaaggcaggtctctgggctgtaactgacgctgagaagcgaaagcatggggagcgaacagg |
| | | attagataccctggtagtccatgccgtaaacgagggcactaggtgtgggggacattccacgttttccgcgccgtagct |
| | | aacgcattaagtgccccgcctggggagtacggccgcaaggctaaaactcaaagaaattgacgggggcccgcacaa |
| | | gcggcggagcatgcggattaattcgatgcaacgcgaagaaccttaccaaggcttgacatatactggactgcgtcaga |
| | | gatggcgtttcccttcggggctggtatacaggtggtgcatggttgtcgtcagctcgtgtcgtgagatgttgggttaagtc |
| | | ccgcaacgagcgcaaccctcgttctatgagccagcacgtgatggtggggactcataggagactgccggggtcaact |
| | | cggaggaaggtggggatgacgtcaaatcatcatgccccttatgtcagggcttcacgcatgctacaatggccggtaca |
| | | gagggttgcgatactgtgaggtggagctaatccctaaaagccggtctcagttcggattggggtctgcaactcgacccc |
| | | atgaagtcggagtcgctagtaatcgcagatcagcaacgctgcggtgaatacgttcccgggccttgtacacaccgccc |
| | | gtcaagtcacgaaagttggtaacacccgaagccggtggcctaaccctggtggggggagccgtcgaaggtgggact |
| | | ggcgattgggactaagtcgtaacaaggtagccgtaccggaaggtgcggctggatcacctccttt |
| Megasphaera_ | SEQ ID | atggagagtttgatcctggctcaggacgaacgctggcggcgtgcttaacacatgcaagtcgaacgagagcgtagag |
| micronuciformis | NO: 310 | aaagcttgctddctacaatctagtggcaaacgggtgagtaacgcgtaaacaacctgccctacggatggggacaaca |
| | | gctggaaacggctgctaataccgaatacgttccgaaagtcgcatgactgtcggaagaaaggatggcctctatttataa |
| | | gctatcgccggaggaggggtttgcgtctgattagctagttggaggggtaacggcccaccaaggcgacgatcagtag |
| | | ccggtctgagaggatgaacggccacattggaactgagacacggtccagactcctacgggaggcagcagtgggga |
| | | atcttccgcaatgggcgaaagcctgacggagcaacgccgcgtgagtgaagacggccttcgggttgtaaagctctgtt |
| | | atacgggacgaataatcttgtggttaatacccataagaagtgacggtaccgtaagagaaagccacggctaactacgt |
| | | gccagcagccgcggtaatacgtaggtggcaagcgttgtccggaattattgggcgtaaagggcgcgcaggcggcttc |
| | | ttaagtctgtcttaaaagtgcggggcttaaccccgtgatgggatggaaactgggaagctcagagtatcggagaggaa |
| | | agcggaattcctagtgtagcggtgaaatgcgtagatattaggaggaacaccagtggcgaaagcggctdctggacga |
| | | aaactgacgctgaggcgcgaaagccaggggagcgaacgggattagataccccggtagtcctggccgtaaacgat |
| | | gggtactaggtgtaggaggtatcgaccccttctgtgccggagttaacgcaataagtaccccgcctggggagtacggc |
| | | cgcaaggagaaactcaaaggaattgacgggggcccgcacaagcggtggagtatgtggataattcgacgcaacgc |
| | | gaagaaccttaccaagccttgacattgatcgcaaggagtagagatactctgttcacttcggaagacgagaaaacagg |
| | | tggtgcacggctgtcgtcagctcgtgtcgtgagatgttgggttaagtcccgcaacgagcgcaacccctatcttctgtta |
| | | ccagcacgtaacggtggggactcaggagagactgccgcagacaatgcggaggaaggcggggatgacgtcaagt |
| | | catcatgccccttatggcttgggctacacacgtactacaatggctcttaatagagggaggcgaaggagcgatccgga |
| | | gcaaaccccaaaaacagagtctcagttcggattgtaggctgcaactcgcctacatgaagcaggaatcgctagtaatc |
| | | gcaggtcagcatactgcggtgaatacgttcccgggccttgtacacaccgcccgtcacaccacgaaagtcattcacac |
| | | ccgaagccggtgaggtaaccgcaaggagccagccgtcgaaggtgggggcgatgattggggtgaagtcgtaacaa |
| | | ggtagccgtatcggaaggtgcggctgg |
| Veillonella_atypica | SEQ ID | ttggagagtttgatcctggctcaggacgaacgctggcggcgtgcttaacacatgcaagtcgaacgaagagcgatgg |
| | NO: 311 | aagcttgcactatcaatcttagtggcgaacgggtgagtaacgcgtaatcaacctgcccttcagagggggacaacagt |
| | | tggaaacgactgctaataccgcatacgatccaatctcggcatcgagactggatgaaaggtggcctctatttataagcta |
| | | tcactgaaggaggggattgcgtctgattagctagttggaggggtaacggcccaccaaggcgatgatcagtagccgg |
| | | tctgagaggatgaacggccacattgggactgagacacggcccagactcctacgggaggcagcagtggggaatat |
| | | ccgcaatggacgaaagtctgacggagcaacgccgcgtgagtgatgacggccttcgggttgtaaagctctgttaatcg |
| | | ggacgaatggttcttgtgcgaatagtgcgaggatttgacggtaccggaatagaaagccacggctaactacgtgccag |
| | | cagccgcggtaatacgtaggtggcaagcgttgtccggaattattgggcgtaaagcgcgcgcaggcggattagttagt |
| | | ctgtataaaagttcggggcttaaccccgtgatgggatggaaactgctgatctagagtatcggagaggaaagtggaat |
| | | tcctagtgtagcggtgaaatgcgtagatattaggaagaacaccagtggcgaaggcgactttctggacgaaaactgac |
| | | gctgaggcgcgaaagccaggggagcgaacgggattagataccccggtagtcctggccgtaaacgatgggtactag |
| | | gtgtaggaggtatcgaccccactgtgccggagttaacgcaataagtaccccgcctggggagtacgaccgcaaggtt |
| | | gaaactcaaaggaattgacgggggcccgcacaagcggtggagtatgtggtttaattcgacgcaacgcgaagaacct |
| | | taccaggtcttgacattgatggacagaactagagatagttcctatcttcggaagccagaaaacaggtggtgcacggtt |
| | | gtcgtcagctcgtgtcgtgagatgttgggttaagtcccgcaacgagcgcaacccctatcttatgttgccagcacttcgg |
| | | gtgggaactcatgagagactgccgcagacaatgcggaggaaggcggggatgacgtcaaatcatcatgccccttatg |
| | | acctgggctacacacgtactacaatgggagttaatagacggaagcgaaaccgcgaggtggagcaaacccgagaaa |
| | | cactctctcagttcggatcgtaggctgcaactcgcctacgtgaagtcggaatcgctagtaatcgcaggtcagcatactg |
| | | cggtgaatacgacccgggccagtacacaccgcccgtcacaccacgaaagtcggaagtgcccaaagccggtggg |
| | | gtaaccttcgggagccagccgtctaaggtaaagtcgatgattggggtgaagtcgtaacaaggtagccgtatcggaag |
| | | gtgcggctggatcacctccat |
| Streptococcus_ | SEQ ID | aatgagagtttgatcctggctcaggacgaacgctggcggcgtgcctaatacatgcaagtagaacgctgaagagagg |
| thermophilus | NO: 312 | agcttgctatcttggatgagttgcgaacgggtgagtaacgcgtaggtaacctgccagtagcgggggataactattgg |
| | | aaacgatagctaataccgcataacaatggatgacacatgtcatttatttgaaaggggcaattgctccactacaagatgg |
| | | acctgcgttgtattagctagtaggtgaggtaatggctcacctaggcgacgatacatagccgacctgagagggtgatcg |
| | | gccacactgggactgagacacggcccagactcctacgggaggcagcagtagggaatcttcggcaatgggggcaa |
| | | ccctgaccgagcaacgccgcgtgagtgaagaaggttttcggatcgtaaagctctgttgtaagtcaagaacgggtgtg |
| | | agagtggaaagttcacactgtgacggtagcttaccagaaagggacggctaactacgtgccagcagccgcggtaata |
| | | cgtaggtcccgagcgagtccggatttattgggcgtaaagcgagcgcaggcgggttgataagtctgaagttaaaggct |
| | | ggggctcaaccatagttcgctttggaaactgtcaaacttgagtgcagaaggggagagtggaattccatgtgtagcggt |
| | | gaaatgcgtagatatatggaggaacaccggtggcgaaagcggctctctggtctgtaactgacgctgaggctcgaaa |
| | | gcgtggggagcgaacaggattagataccctggtagtccacgccgtaaacgatgagtgctaggtgttggatcctttcc |
| | | gggattcagtgccgaagctaacgcattaagcactccgcctggggagtacgaccgcaaggttgaaactcaaaggaat |
| | | tgacgggggcccgcacaagcggtggagcatgtggtttaattcgaagcaacgcgaagaaccttaccaggtcttgacat |
| | | cccgatgctatttctagagatagaaagttacttcggtacatcggtgacaggtggtgcatggttgtcgtcagctcgtgtcg |
| | | tgagatgttgggttaagtcccgcaacgagcgcaacccctattgttagagccatcattcagttgggcactctagcgaga |
| | | ctgccggtaataaaccggaggaaggtggggatgacgtcaaatcatcatgccccttatgacctgggctacacacgtgc |
| | | tacaatggttggtacaacgagttgcgagtcggtgacggcgagctaatctcttaaagccaatctcagttcggattgtagg |
| | | ctgcaactcgcctacatgaagtcggaatcgctagtaatcgcggatcagcacgccgcggtgaatacgttcccgggcct |
| | | tgtacacaccgcccgtcacaccacgagagtttgtaacacccgaagtcggtgaggtaaccattggagccagccgcct |
| | | aaggtgggacagatgattggggtgaagtcgtaacaaggtagccgtatcggaaggtgcggctgggtcacctcctt |
| Streptococcus_ | SEQ ID | atgaaagtttgatcctggctcaggacgaacgctggcggcgtgcctaatacatgcaagtagaacgctgaagagaggag |
| thermophilus | NO: 313 | cttgctcacttggatgagttgcgaacgggtgagtaacgcgtaggtaacctgccttgtagcgggggataactattggaaa |
| | | cgatagctaataccgcataacaatggatgacacatgtcatttatttgaaaggggcaattgctccactacaagatggacctg |
| | | cgttgtattagctagtaggtgaggtaatggctcacctaggcgacgatacatagccgacctgagagggtgatcggccac |
| | | actgggactgagacacggcccagactcctacgggaggcagcagtagggaatcacggcaatgggggcaaccctgac |
| | | cgagcaacgccgcgtgagtgaagaaggttttcggatcgtaaagctctgagtaagtcaagaacgggtgtgagagtgga |
| | | aagttcacactgtgacggtagcttaccagaaagggacggctaactacgtgccagcagccgcggtaatacgtaggtccc |
| | | gagcgttgtccggatttattgggcgtaaagcgagcgcaggcggtttgataagtctgaagttaaaggctgtggctcaacc |
| | | atagttcgctttggaaactgtcaaacttgagtgcagaaggggagagtggaattccatgtgtagcggtgaaatgcgtagat |
| | | atatggaggaacaccggtggcgaaagcggctcactggtctgtaactgacgctgaggctcgaaagcgtggggagcga |
| | | acaggattagataccctggtagtccacgccgtaaacgatgagtgctaggtgttggatcctttccgggattcagtgccgca |
| | | gctaacgcattaagcactccgcctggggagtacgaccgcaaggttgaaactcaaaggaattgacgggggcccgcac |
| | | aagcggtggagcatgtggtttaattcgaagcaacgcgaagaaccttaccaggtcttgacatcccgatgctatactagag |
| | | atagaaagttacttcggtacatcggtgacaggtggtgcatggttgtcgtcagctcgtgtcgtgagatgttgggttaagtcc |
| | | cgcaacgagcgcaacccctattgttagttgccatcattcagttgggcactctagcgagactgccggtaataaaccggag |
| | | gaaggtggggatgacgtcaaatcatcatgccccttatgacctgggctacacacgtgctacaatggttggtacaacgagtt |
| | | gcgagtcggtgacggcgagctaatctcttaaagccaatctcagttcggattgtaggctgcaactcgcctacatgaagtcg |
| | | gaatcgctagtaatcgcggatcagcacgccgcggtgaatacgttcccgggccttgtacacaccgcccgtcacaccacg |
| | | agagatgtaacacccgaagtcggtgaggtaaccattggagccagccgcctaaggtgggacagatgattggggtgaa |
| | | gtcgtaacaaggtagccgtatcggaaggtgcggctggatcacctcctt |
| Streptococcus_ | SEQ ID | aatgagagtagatcctggctcaggacgaacgctggcggcgtgcctaatacatgcaagtagaacgctgaagagagga |
| thermophilus | NO: 314 | gcttgctcttcttggatgagttgcgaacgggtgagtaacgcgtaggtaacctgccttgtagcgggggataactattggaa |
| | | acgatagctaataccgcataacaatggatgacacatgtcatttatttgaaaggggcaattgctccactacaagatggacct |
| | | gcgttgtattagctagtaggtgaggtaatggctcacctaggcgacgatacatagccgacctgagagggtgatcggcca |
| | | cactgggactgagacacggcccagactcctacgggaggcagcagtagggaatcttcggcaatgggggcaaccctga |
| | | ccgagcaacgccgcgtgagtgaagaaggttttcggatcgtaaagctctgttgtaagtcaagaacgggtgtgagagtgg |
| | | aaagttcacactgtgacggtagcttaccagaaagggacggctaactacgtgccagcagccgcggtaatacgtaggtcc |
| | | cgagcgttgtccggatttattgggcgtaaagcgagcgcaggcggtttgataagtctgaagttaaaggctgtggctcaac |
| | | catagttcgctttggaaactgtcaaacttgagtgcagaaggggagagtggaattccatgtgtagcggtgaaatgcgtag |
| | | atatatggaggaacaccggtggcgaaagcggctctctggtctgtaactgacgctgaggctcgaaagcgtggggagcg |
| | | aacaggattagataccctggtagtccacgccgtaaacgatgagtgctaggtgttggatcctttccgggattcagtgccgc |
| | | agctaacgcattaagcactccgcctggggagtacgaccgcaaggttgaaactcaaaggaattgacgggggcccgca |
| | | caagcggtggagcatgtggtttaattcgaagcaacgcgaagaaccttaccaggtatgacatcccgatgctatttctaga |
| | | gatagaaagttacttcggtacatcggtgacaggtggtgcatggttgtcgtcagctcgtgtcgtgagatgttgggttaagtc |
| | | ccgcaacgagcgcaacccctattgttagagccatcattcagttgggcactctagcgagactgccggtaataaaccgga |
| | | ggaaggtggggatgacgtcaaatcatcatgccccttatgacctgggctacacacgtgctacaatggttggtacaacgag |
| | | ttgcgagtcggtgacggcgagctaatctcttaaagccaatctcagttcggattgtaggctgcaactcgcctacatgaagt |
| | | cggaatcgctagtaatcgcggatcagcacgccgcggtgaatacgacccgggccagtacacaccgcccgtcacacca |
| | | cgagagtttgtaacacccgaagtcggtgaggtaaccttttggagccagccgcctaaggtgggacagatgattggggtg |
| | | aagtcgtaacaaggtagccgtatcggaaggtgcggctggatcacctcctt |
| Streptococcus_ | SEQ ID | aatgagagtttgatcctggctcaggacgaacgctggcggcgtgcctaatacatgcaagtagaacgctgaagagagga |
| thermophilus | NO: 315 | gcttgctcttcttggatgagttgcgaacgggtgagtaacgcgtaggtaacctgccttgtagcgggggataactattggaa |
| | | acgatagctaataccgcataacaatggatgacacatgtcatttatttgaaaggggcaattgctccactacaagatggacct |
| | | gcgttgtattagctagtaggtgaggtaatggctcacctaggcgacgatacatagccgacctgagagggtgatcggcca |
| | | cactgggactgagacacggcccagactcctacgggaggcagcagtagggaatcttcggcaatgggggcaaccctga |
| | | ccgagcaacgccgcgtgagtgaagaaggttttcggatcgtaaagctctgttgtaagtcaagaacgggtgtgagagtgg |
| | | aaagttcacactgtgacggtagcttaccagaaagggacggctaactacgtgccagcagccgcggtaatacgtaggtcc |
| | | cgagcgttgtccggatttattgggcgtaaagcgagcgcaggcgggttgataagtctgaagttaaaggctggggctcaa |
| | | ccatagttcgctttggaaactgtcaaacttgagtgcagaaggggagagtggaattccatgtgtagcggtgaaatgcgta |
| | | gatatatggaggaacaccggtggcgaaagcggctctctggtctgtaactgacgctgaggctcgaaagcgtggggagc |
| | | gaacaggattagataccctggtagtccacgccgtaaacgatgagtgctaggtgaggatcctttccgggattcagtgccg |
| | | cagctaacgcattaagcactccgcctggggagtacgaccgcaaggttgaaactcaaaggaattgacgggggcccgc |
| | | acaagcggtggagcatgtggtttaattcgaagcaacgcgaagaaccttaccaggtatgacatcccgatgctatttctag |
| | | agatagaaagttacttcggtacatcggtgacaggtggtgcatggttgtcgtcagctcgtgtcgtgagatgttgggttaagt |
| | | cccgcaacgagcgcaacccctattgttagttgccatcattcagttgggcactctagcgagactgccggtaataaaccgg |
| | | aggaaggtggggatgacgtcaaatcatcatgcccatatgacctgggctacacacgtgctacaatggttggtacaacga |
| | | gttgcgagtcggtgacggcgagctaatctcttaaagccaatctcagttcggattgtaggctgcaactcgcctacatgaag |
| | | tcggaatcgctagtaatcgcggatcagcacgccgcggtgaatacgttcccgggccttgtacacaccgcccgtcacacc |
| | | acgagagtttgtaacacccgaagtcggtgaggtaaccattggagccagccgcctaaggtgggacagatgattggggt |
| | | gaagtcgtaacaaggtagccgtatcggaaggtgcggctggtcacctcctt |
| Streptococcus_ | SEQ ID | aatgagagtttgatcctggctcaggacgaacgctggcggcgtgcctaatacatgcaagtagaacgctgaagagagga |
| thermophilus | NO: 316 | gcttgctcttcttggatgagttgcgaacgggtgagtaacgcgtaggtaacctgccttgtagcgggggataactattggaa |
| | | acgatagctaataccgcataacaatggatgacacatgtcatttatttgaaaggggcaattgctccactacaagatggacct |
| | | gcgttgtattagctagtaggtgaggtaatggctcacctaggcgacgatacatagccgacctgagagggtgatcggcca |
| | | cactgggactgagacacggcccagactcctacgggaggcagcagtagggaatcttcggcaatgggggcaaccctga |
| | | ccgagcaacgccgcgtgagtgaagaaggttttcggatcgtaaagctctgttgtaagtcaagaacgggtgtgagagtgg |
| | | aaagttcacactgtgacggtagcttaccagaaagggacggctaactacgtgccagcagccgcggtaatacgtaggtcc |
| | | cgagcgttgtccggatttattgggcgtaaagcgagcgcaggcgggttgataagtctgaagttaaaggctgtggctcaac |
| | | catagttcgattggaaactgtcaaacttgagtgcagaaggggagagtggaattccatgtgtagcggtgaaatgcgtag |
| | | atatatggaggaacaccggtggcgaaagcggctctctggtctgtaactgacgctgaggctcgaaagcgtggggagcg |
| | | aacaggattagataccctggtagtccacgccgtaaacgatgagtgctaggtgttggatcctttccgggattcagtgccgc |
| | | agctaacgcattaagcactccgcctggggagtacgaccgcaaggttgaaactcaaaggaattgacgggggcccgca |
| | | caagcggtggagcatgtggtttaattcgaagcaacgcgaagaaccttaccaggtatgacatcccgatgctatttctaga |
| | | gatagaaagttacttcggtacatcggtgacaggtgggcatggttgtcgtcagctcgtgtcgtgagatgttgggttaagtcc |
| | | cgcaacgagcgcaacccctattgttagttgccatcattcagagggcactctagcgagactgccggtaataaaccggag |
| | | gaaggtggggatgacgtcaaatcatcatgccccttatgacctgggctacacacgtgctacaatggttggtacaacgagtt |
| | | gcgagtcggtgacggcgagctaatctcttaaagccaatctcagttcggattgtaggctgcaactcgcctacatgaagtcg |
| | | gaatcgctagtaatcgcggatcagcacgccgcggtgaatacgttcccgggccagtacacaccgcccgtcacaccacg |
| | | agagtttgtaacacccgaagtcggtgaggtaaccattggagccagccgcctaaggtgggacagatgattggggtgaa |
| | | gtcgtaacaaggtagccgtatcggaaggtgcggctgggtcacctcctt |
| Lactobacillus_ | SEQ ID | attgagagatgatcctggctcaggatgaacgctggcggcgtgcctaatacatgcaagtcgaacgaaacttcatatcacc |
| animalis | NO: 317 | gagtgcttgcactcaccgataaagagttgagtggcgaacgggtgagtaacacgtgggcaacctgcccaaaagaggg |
| | | ggataacacttggaaacaggtgctaataccgcataaccatagttaccgcatggtaactatgtaaaaggtggctatgctac |
| | | cgcttttggatgggcccgcggcgcattagctagttggtgaggtaaaggcttaccaaggcaatgatgcgtagccgaactg |
| | | agaggagatcggccacattgggactgagacacggcccaaactcctacgggaggcagcagtagggaatcaccacaa |
| | | tgggcgaaagcctgatggagcaacgccgcgtgggtgaagaaggtcttcggatcgtaaaaccctgagttagagaaga |
| | | aagtgcgtgagagtaactgttcacgatcgacggtatctaaccagaaagccacggctaactacgtgccagcagccgcg |
| | | gtaatacgtaggtggcaagcgttatccggatttattgggcgtaaagggaacgcaggcggtcdttaagtctgatgtgaaa |
| | | gccttcggcttaaccggagtagtgcattggaaactgggagacttgagtgcagaagaggagagtggaactccatgtgta |
| | | gcggtgaaatgcgtagatatatggaagaacaccagtggcgaaagcggctctctggtctgtaactgacgctgaggdcg |
| | | aaagcgtgggtagcaaacaggattagataccctggtagtccacgccgtaaacgatgaatgctaagtgttggagggtttc |
| | | cgcccttcagtgctgcagctaacgcaataagcattccgcctggggagtacgaccgcaaggttgaaactcaaaggaatt |
| | | gacgggggcccgcacaagcggtggagcatgtggtttaattcgaagcaacgcgaagaaccttaccaggtatgacatct |
| | | tctgacaatcctagagataggactttcccttcggggacagaatgacaggtggtgcatggagtcgtcagacgtgtcgtg |
| | | agatgttgggttaagtcccgcaacgagcgcaacccttattgttagttgccagcattaagttgggcactctagcaagactg |
| | | ccggtgacaaaccggaggaaggtggggatgacgtcaaatcatcatgccccttatgacctgggctacacacgtgctaca |
| | | atggacggtacaacgagtcgcaagaccgcgaggtttagcaaatctcttaaagccgttctcagttcggattgtaggctgca |
| | | actcgcctacatgaagtcggaatcgctagtaatcgcggatcagcatgccgcggtgaatacgttcccgggccttgtacac |
| | | accgcccgtcacaccatgagagtttgtaacacccaaagccggtggggtaaccttttggagccagccgtctaaggtggg |
| | | acagatgattggggtgaagtcgtaacaaggtagccgtaggagaacctgcggctggatcacctccttt |
| Bilophila_ | SEQ ID | ctggagagtttgattctggctcagattgaacgctggcggcgtgcttaacacatgcaagtcgaacgtgaaagtccttcggg |
| wadsworthia | NO: 318 | gcgagtaaagtggcgcacgggtgagtaacgcgtggataatctacccttaagatggggataacggctggaaacggtcg |
| | | ctaataccgaatacgctcccgattttatcattggggggaaagatggcctctgcttgcaagctatcgcttaaggatgagtcc |
| | | gcgtcccattagctagaggcggggtaacggcccaccaaggcgacgatgggtagccggtctgagaggatgaccggc |
| | | cacactggaactggaacacggtccagactcctacgggaggcagcagtggggaatattgcgcaatgggcgaaagcct |
| | | gacgcagcgacgccgcgtgagggatgaaggttctcggatcgtaaacctctgtcaggggggaagaaaccccctcgtgt |
| | | gaataatgcgagggcttgacggtacccccaaaggaagcaccggctaactccgtgccagcagccgcggtaatacgga |
| | | gggtgcaagcgttaatcggaatcactgggcgtaaagcgcacgtaggcggcaggtaagtcaggggtgaaatcccaca |
| | | gcccaactgtggaactgcctttgatactgccaggcttgagtaccggagagggtggcggaattccaggtgtaggagtga |
| | | aatccgtagatatctggaggaacaccggtggcgaaggcggccacctggacggtaactgacgctgaggtgcgaaagc |
| | | gtgggtagcaaacaggattagataccctggtagtccacgctgtaaacgatgggtgctgggtgctgggatgtatgtctcg |
| | | gtgccgtagctaacgcgataagcaccccgcctggggagtacggtcgcaaggctgaaactcaaagaaattgacgggg |
| | | gcccgcacaagcggtggagtatgtggtttaattcgatgcaacgcgaagaaccttacccaggcttgacatctagggaac |
| | | ccttcggaaatgaaggggtgcccttcggggagccctaagacaggtgctgcatggctgtcgtcagctcgtgccgtgagg |
| | | tgttgggttaagtcccgcaacgagcgcaacccctatcttcagttgccagcaggtaaggctgggcactctggagagacc |
| | | gccccggtcaacggggaggaaggtggggacgacgtcaagtcatcatggcccttacgcctggggctacacacgtact |
| | | acaatggcgcgcacaaagggtagcgagaccgcgaggtggagccaatcccaaaaaacgcgtcccagtccggattgg |
| | | agtctgcaactcgactccatgaagtcggaatcgctagtaattcgagatcagcatgctcgggtgaatgcgttcccgggcct |
| | | tgtacacaccgcccgtcacaccacgaaagtcggattacccgaagccggtgagctaactcgcaagaggagcagccgt |
| | | ctacggtagggccgatgattggggtgaagtcgtaacaaggtagccgtaggggaacctgcggctggatcacctccttt |
| Bilophila_ | SEQ ID | ctggagagtttgattctggctcagattgaacgctggcggcgtgcttaacacatgcaagtcgaacgtgaaagtccttcggg |
| wadsworthia | NO: 319 | gcgagtaaagtggcgcacgggtgagtaacgcgtggataatctacccttaagatggggataacggctggaaacggtcg |
| | | ctaataccgaatacgctcccgattttatcgttggggggaaagatggcctctgcttgcaagctatcgcttaaggatgagtcc |
| | | gcgtcccattagctagaggcggggtaacggcccaccaaggcgacgatgggtagccggtctgagaggatgaccggc |
| | | cacactggaactggaacacggtccagactcctacgggaggcagcagtggggaatattgcgcaatgggcgaaagcct |
| | | gacgcagcgacgccgcgtgagggatgaaggactcggatcgtaaacctctgtcaggggggaagaaaccccctcgtgt |
| | | gaataatgcgagggcttgacggtacccccaaaggaagcaccggctaactccgtgccagcagccgcggtaatacgga |
| | | gggtgcaagcgttaatcggaatcactgggcgtaaagcgcacgtaggcggcttggtaagtcaggggtgaaatcccaca |
| | | gcccaactgtggaactgcctttgatactgccaggcttgagtaccggagagggtggcggaattccaggtgtaggagtga |
| | | aatccgtagatatctggaggaacaccggtggcgaaggcggccacctggacggtaactgacgctgaggtgcgaaagc |
| | | gtgggtagcaaacaggattagataccctggtagtccacgctgtaaacgatgggtgctgggtgctgggatgtatgtctcg |
| | | gtgccgtagctaacgcgataagcaccccgcctggggagtacggtcgcaaggctgaaactcaaagaaattgacgggg |
| | | gcccgcacaagcggtggagtatgtggtttaattcgatgcaacgcgaagaaccttacccaggcttgacatctagggaac |
| | | ccttcggaaatgaaggggtgcccttcggggagccctaagacaggtgctgcatggctgtcgtcagctcgtgccgtgagg |
| | | tgttgggttaagtcccgcaacgagcgcaacccctatcttcagttgccagcaggtaaggctgggcactctggagagacc |
| | | gccccggtcaacggggaggaaggtggggacgacgtcaagtcatcatggcccttacgcctggggctacacacgtact |
| | | acaatggcgcgcacaaagggtagcgagaccgcgaggtggagccaatcccaaaaaacgcgtcccagtccggattgg |
| | | agtctgcaactcgactccatgaagtcggaatcgctagtaattcgagatcagcatgctcgggtgaatgcgttcccgggcct |
| | | tgtacacaccgcccgtcacaccacgaaagtcggttttacccgaagccggtgagctaactcgcaagaggagcagccgt |
| | | ctacggtagggccgatgattggggtgaagtcgtaacaaggtagccgtaggggaacctgcggctggatcacctccttt |
| Bilophila_ | SEQ ID | ctggagagtttgattctggctcagattgaacgctggcggcgtgcttaacacatgcaagtcgaacgtgaaagtccttcggg |
| wadsworthia | NO: 320 | gcgagtaaagtggcgcacgggtgagtaacgcgtggataatctacccttaagatggggataacggctggaaacggtcg |
| | | ctaataccgaatacgctcccgattttatcattggggggaaagatggcctctgcttgcaagctatcgcttaaggatgagtcc |
| | | gcgtcccattagctagttggcggggtaacggcccaccaaggcgacgatgggtagccggtctgagaggatgaccggc |
| | | cacactggaactggaacacggtccagactcctacgggaggcagcagtggggaatattgcgcaatgggcgaaagcct |
| | | gacgcagcgacgccgcgtgagggatgaaggttctcggatcgtaaacctctgtcaggggggaagaaaccccctcgtgt |
| | | gaataatgcgagggcttgacggtacccccaaaggaagcaccggctaactccgtgccagcagccgcggtaatacgga |
| | | gggtgcaagcgttaatcggaatcactgggcgtaaagcgcacgtaggcggcaggtaagtcaggggtgaaatcccaca |
| | | gcccaactgtggaactgcctttgatactgccaggcttgagtaccggagagggtggcggaattccaggtgtaggagtga |
| | | aatccgtagatatctggaggaacaccggtggcgaaggcggccacctggacggtaactgacgctgaggtgcgaaagc |
| | | gtgggtagcaaacaggattagataccctggtagtccacgctgtaaacgatgggtgctgggtgctgggatgtatgtctcg |
| | | gtgccgtagctaacgcgataagcaccccgcctggggagtacggtcgcaaggctgaaactcaaagaaattgacgggg |
| | | gcccgcacaagcggtggagtatgtgotaattcgatgcaacgcgaagaaccttacccaggcttgacatctagggaac |
| | | ccttcggaaatgaaggggtgcccttcggggagccctaagacaggtgctgcatggctgtcgtcagctcgtgccgtgagg |
| | | tgttgggttaagtcccgcaacgagcgcaacccctatcttcagttgccagcaggtaaggctgggcactctggagagacc |
| | | gccccggtcaacggggaggaaggtggggacgacgtcaagtcatcatggcccttacgcctggggctacacacgtact |
| | | acaatggcgcgcacaaagggtagcgagaccgcgaggtggagccaatcccaaaaaacgcgtcccagtccggattgg |
| | | agtctgcaactcgactccatgaagtcggaatcgctagtaattcgagatcagcatgctcgggtgaatgcgttcccgggcct |
| | | tgtacacaccgcccgtcacaccacgaaagtcggttttacccgaagccggtgagctaactcgcaagaggagcagccgt |
| | | ctacggtagggccgatgattggggtgaagtcgtaacaaggtagccgtaggggaacctgcggctggatcacctccttt |
| Bilophila_ | SEQ ID | ctggagagtttgattctggctcagattgaacgctggcggcgtgcttaacacatgcaagtcgaacgtgaaagtccttcggg |
| wadsworthia | NO: 321 | gcgagtaaagtggcgcacgggtgagtaacgcgtggataatctacccttaagatggggataacggctggaaacggtcg |
| | | ctaataccgaatacgctcccgattttatcgttggggggaaagatggcctctgcttgcaagctatcgcttaaggatgagtcc |
| | | gcgtcccattagctagttggcggggtaacggcccaccaaggcgacgatgggtagccggtctgagaggatgaccggc |
| | | cacactggaactggaacacggtccagactcctacgggaggcagcagtggggaatattgcgcaatgggcgaaagcct |
| | | gacgcagcgacgccgcgtgagggatgaaggttctcggatcgtaaacctctgtcaggggggaagaaaccccctcgtgt |
| | | gaataatgcgagggcttgacggtacccccaaaggaagcaccggctaactccgtgccagcagccgcggtaatacgga |
| | | gggtgcaagcgttaatcggaatcactgggcgtaaagcgcacgtaggcggcttggtaagtcaggggtgaaatcccaca |
| | | gcccaactgtggaactgcctttgatactgccaggcttgagtaccggagagggtggcggaattccaggtgtaggagtga |
| | | aatccgtagatatctggaggaacaccggtggcgaaggcggccacctggacggtaactgacgctgaggtgcgaaagc |
| | | gtgggtagcaaacaggattagataccctggtagtccacgctgtaaacgatgggtgctgggtgctgggatgtatgtctcg |
| | | gtgccgtagctaacgcgataagcaccccgcctggggagtacggtcgcaaggctgaaactcaaagaaattgacgggg |
| | | gcccgcacaagcggtggagtatgtggtttaattcgatgcaacgcgaagaaccttacccaggcttgacatctagggaac |
| | | ccttcggaaatgaaggggtgcccttcggggagccctaagacaggtgctgcatggctgtcgtcagctcgtgccgtgagg |
| | | tgttgggttaagtcccgcaacgagcgcaacccctatcttcagttgccagcaggtaaggctgggcactctggagagacc |
| | | gccccggtcaacggggaggaaggtggggacgacgtcaagtcatcatggcccttacgcctggggctacacacgtact |
| | | acaatggcgcgcacaaagggtagcgagaccgcgaggtggagccaatcccaaaaaacgcgtcccagtccggattgg |
| | | agtctgcaactcgactccatgaagtcggaatcgctagtaattcgagatcagcatgctcgggtgaatgcgttcccgggcct |
| | | tgtacacaccgcccgtcacaccacgaaagtcggttttacccgaagccggtgagctaactcgcaagaggagcagccgt |
| | | ctacggtagggccgatgattggggtgaagtcgtaacaaggtagccgtaggggaacctgcggctggatcacctccttt |
| Veillonella_parvula | SEQ ID | ttggagagatgatcctggctcaggacgaacgctggcggcgtgcttaacacatgcaagtcgaacgaagagcgatggaa |
| | NO: 322 | gcttgcttctatcaatcttagtggcgaacgggtgagtaacgcgtaatcaacctgcccttcagagggggacaacagttgg |
| | | aaacgactgctaataccgcatacgatctaatctcggcatcgaggatagatgaaaggtggcctctacatgtaagctatcac |
| | | tgaaggaggggattgcgtctgattagctagttggaggggtaacggcccaccaaggcgatgatcagtagccggtctga |
| | | gaggatgaacggccacattgggactgagacacggcccagactcctacgggaggcagcagtggggaatcttccgcaa |
| | | tggacgaaagtctgacggagcaacgccgcgtgagtgatgacggccttcgggttgtaaagctctgttaatcgggacgaa |
| | | aggccttcttgcgaacagttagaaggattgacggtaccggaatagaaagccacggctaactacgtgccagcagccgc |
| | | ggtaatacgtaggtggcaagcgctgtccggaattattgggcgtaaagcgcgcgcaggcggatcagtcagtctgtcttaa |
| | | aagttcggggcttaaccccgtgatgggatggaaactgctgatctagagtatcggagaggaaagtggaattcctagtgta |
| | | gcggtgaaatgcgtagatattaggaagaacaccagtggcgaaggcgactttctggacgaaaactgacgctgaggcgc |
| | | gaaagccaggggagcgaacgggattagataccccggtagtcctggccgtaaacgatgggtactaggtgtaggaggt |
| | | atcgaccccttctgtgccggagttaacgcaataagtaccccgcctggggagtacgaccgcaaggttgaaactcaaagg |
| | | aattgacgggggcccgcacaagcggtggagtatgtggtttaattcgacgcaacgcgaagaaccttaccaggtcttgac |
| | | attgatggacagaactagagatagttcctcttcttcggaagccagaaaacaggtggtgcacggttgtcgtcagctcgtgt |
| | | cgtgagatgttgggttaagtcccgcaacgagcgcaacccctatcttatgttgccagcacgtaatggtgggaactcatga |
| | | gagactgccgcagacaatgcggaggaaggcggggatgacgtcaaatcatcatgccccttatgacctgggctacacac |
| | | gtactacaatgggagttaatagacggaagcgagatcgcgagatggagcaaacccgagaaacactctctcagttcggat |
| | | cgtaggctgcaactcgcctacgtgaagtcggaatcgctagtaatcgcaggtcagcatactgcggtgaatacgttcccgg |
| | | gccttgtacacaccgcccgtcacaccacgaaagtcggaagtgcccaaagccggtggggtaaccttcgggagccagc |
| | | cgtctaaggtaaagtcgatgattggggtgaagtcgtaacaaggtagccgtatcggaaggtgcggctggatcacctcctt |
| | | t |
| Lachnospiraceae_ | SEQ ID | agagagatgatcctggctcaggatgaacgctggcggcgtgcctaacacatgcaagtcgaacgaagttacacggagg |
| bacterium_3_1_57FAA_ | NO: 323 | aagttttcggatggaatcggtataacttagtggcggacgggtgagtaacgcgtgggaaacctgccctgtaccggggga |
| CT1 | | taacacttagaaataggtgctaataccgcataagcgcacggaaccgcatggttccgtgtgaaaaactccggtggtacag |
| | | gatggtcccgcgtctgattagccagttggcagggtaacggcctaccaaagcgacgatcagtagccggcctgagaggg |
| | | tgaacggccacattgggactgagacacggcccaaactcctacgggaggcagcagtggggaatattgcacaatgggg |
| | | gaaaccctgatgcagcgacgccgcgtgagtgaagaagtatttcggtatgtaaagctctatcagcagggaagaaaatga |
| | | cggtacctgactaagaagccccggctaactacgtgccagcagccgcggtaatacgtagggggcaagcgttatccgga |
| | | tttactgggtgtaaagggagcgtagacggcatggcaagccagatgtgaaaacccagggctcaaccttgggattgcattt |
| | | ggaactgccaggctggagtgcaggagaggtaagcggaattcctagtgtagcggtgaaatgcgtagatattaggagga |
| | | acaccagtggcgaaggcggcttactggactgtaactgacgttgaggctcgaaagcgtggggagcaaacaggattaga |
| | | taccctggtagtccacgcggtaaacgatgattgctaggtgtaggtgggtatggacccatcggtgccgcagctaacgca |
| | | ataagcaatccacctggggagtacgttcgcaagaatgaaactcaaaggaattgacggggacccgcacaagcggtgg |
| | | agcatgtggtttaattcgaagcaacgcgaagaaccttaccaagtcttgacatccccctgaatatcttgtaaaaaagagatg |
| | | ccttcgggacaggggagacaggtggtgcatggttgtcgtcagctcgtgtcgtgagatgttgggttaagtcccgcaacga |
| | | gcgcaacccttattcttagtagccagcaggtaaggctgggcactctaaggagactgccggggataacccggaggaag |
| | | gcggggatgacgtcaaatcatcatgccccttatgatttgggctacacacgtgctacaatggcgtaaacaaagggaagc |
| | | gagacagtgatgtggagcaaatcccagaaataacgtctcagttcggattgtagtctgcaactcgactacatgaagctgg |
| | | aatcgctagtaatcgcgaatcagcatgtcgcggtgaatacgttcccgggtcttgtacacaccgcccgtcacaccatggg |
| | | agttggaaatgcccgaagtctgtgacctaaccgaaagggaggagcagccgaaggcaggtctgataactggggtgaa |
| | | gtcgtaacaaggtagccgtatcggaaggtgcggctggatcacctccttt |
| Lachnospiraceae_ | SEQ ID | agagagtttgatcctggctcaggatgaacgctggcggcgtgcctaacacatgcaagtcgaacgaagttatatcgagga |
| bacterium_3_1_57FAA_ | NO: 324 | agttttcggatggaatcagtataacttagtggcggacgggtgagtaacgcgtgggaaacctgccctgtaccgggggat |
| CT1 | | aacacttagaaataggtgctaataccgcataagcgcacagcttcacatgaagcagtgtgaaaaactccggtggtacag |
| | | gatggtcccgcgtctgattagccagttggcagggtaacggcctaccaaagcgacgatcagtagccggcctgagaggg |
| | | tgaacggccacattgggactgagacacggcccaaactcctacgggaggcagcagtggggaatattgcacaatgggg |
| | | gaaaccctgatgcagcgacgccgcgtgagtgaagaagtatttcggtatgtaaagctctatcagcagggaagaaaatga |
| | | cggtacctgactaagaagccccggctaactacgtgccagcagccgcggtaatacgtagggggcaagcgttatccgga |
| | | tttactgggtgtaaagggagcgtagacggcatggcaagccagatgtgaaaacccagggctcaaccttgggattgcattt |
| | | ggaactgtcaggctggagtgcaggagaggtaagcggaattcctagtgtagcggtgaaatgcgtagatattaggagga |
| | | acaccagtggcgaaggcggcttactggactgtaactgacgttgaggctcgaaagcgtggggagcaaacaggattaga |
| | | taccctggtagtccacgcggtaaacgatgattgctaggtgtaggtgggtatggacccatcggtgccgcagctaacgca |
| | | ataagcaatccacctggggagtacgttcgcaagaatgaaactcaaaggaattgacggggacccgcacaagcggtgg |
| | | agcatgtggtttaattcgaagcaacgcgaagaaccttaccaagtcttgacatccccctgactctcctgtaaaagagtgag |
| | | gccttcgggacattggagacaggtggtgcatggttgtcgtcagctcgtgtcgtgagatgttgggttaagtcccgcaacg |
| | | agcgcaacccttattcttagtagccagcaggtaaagctgggcactctaaggagactgccggggataacccggaggaa |
| | | ggcggggatgacgtcaaatcatcatgccccttatgatttgggctacacacgtgctacaatggcgtaaacaaagggaag |
| | | cgagacagtgatgtggagcaaatcccagaaataacgtctcagttcggattgtagtctgcaactcgactacatgaagctg |
| | | gaatcgctagtaatcgcgaatcagcatgtcgcggtgaatacgacccgggtatgtacacaccgcccgtcacaccatgg |
| | | gagttggaaatgcccgaagtctgtgacctaaccgaaagggaggagcagccgaaggcaggtctgataactggggtga |
| | | agtcgtaacaaggtagccgtatcggaaggtgcggctggatcacctccat |
| Lachnospiraceae_ | SEQ ID | agagagtttgatcctggctcaggatgaacgctggcggcgtgcctaacacatgcaagtcgaacgaagttacacagagg |
| bacterium_3_1_57FAA_ | NO: 325 | aagttttcggatggaatcggtataacttagtggcggacgggtgagtaacgcgtgggaaacctgccctgtaccggggga |
| CT1 | | taacacttagaaataggtgctaataccgcataagcgcacggattcacatgaagcagtgtgaaaaactccggtggtacag |
| | | gatggtcccgcgtctgattagccagaggcagggtaacggcctaccaaagcgacgatcagtagccggcctgagaggg |
| | | tgaacggccacattgggactgagacacggcccaaactcctacgggaggcagcagtggggaatattgcacaatgggg |
| | | gaaaccctgatgcagcgacgccgcgtgagtgaagaagtatttcggtatgtaaagctctatcagcagggaagaaaatga |
| | | cggtacctgactaagaagccccggctaactacgtgccagcagccgcggtaatacgtagggggcaagcgttatccgga |
| | | tttactgggtgtaaagggagcgtagacggcatggcaagccagatgtgaaaacccagggctcaaccttgggattgcattt |
| | | ggaactgtcaggctggagtgcaggagaggtaagcggaattcctagtgtagcggtgaaatgcgtagatattaggagga |
| | | acaccagtggcgaaggcggcttactggactgtaactgacgttgaggctcgaaagcgtggggagcaaacaggattaga |
| | | taccctggtagtccacgcggtaaacgatgattgctaggtgtaggtgggtatggacccatcggtgccgcagctaacgca |
| | | ataagcaatccacctggggagtacgttcgcaagaatgaaactcaaaggaattgacggggacccgcacaagcggtgg |
| | | agcatgtggtttaattcgaagcaacgcgaagaaccttaccaagtcttgacatcccaatgacgcacctgtaaagaggtgtt |
| | | cccttcggggcattggagacaggtggtgcatggagtcgtcagctcgtgtcgtgagatgagggttaagtcccgcaacg |
| | | agcgcaacccttattcttagtagccagcaggtaaagctgggcactctaaggagactgccggggataacccggaggaa |
| | | ggcggggatgacgtcaaatcatcatgccccttatgatttgggctacacacgtgctacaatggcgtaaacaaagggaag |
| | | cgagacagtgatgtggagcaaatcccagaaataacgtctcagttcggattgtagtctgcaactcgactacatgaagctg |
| | | gaatcgctagtaatcgcgaatcagcatgtcgcggtgaatacgttcccgggtcttgtacacaccgcccgtcacaccatgg |
| | | gagttggaaatgcccgaagtctgtgacctaaccgaaagggaggagcagccgaaggcaggtctgataactggggtga |
| | | agtcgtaacaaggtagccgtatcggaaggtgcggctggatcacctccat |
| Erysipelotrichaceae_ | SEQ ID | atggagagtttgatcctggctcaggatgaacgctggcggcatgcctaatacatgcaagtcgaacgaagcttcaggaag |
| bacterium_2_2_44A | NO: 326 | cagcttccaaagagacttagtggcgaacgggtgagtaacacgtaggtaacctgcccatgtgtccgggataactgctgg |
| | | aaacggtagctaaaaccggataggtatacagagcgcatgctcagtatattaaagcgcccatcaaggcgtgaacatgga |
| | | tggacctgcggcgcattagctagttggtgaggtaacggcccaccaaggcgatgatgcgtagccggcctgagagggta |
| | | aacggccacattgggactgagacacggcccaaactcctacgggaggcagcagtagggaattttcgtcaatgggggaa |
| | | accctgaacgagcaatgccgcgtgagtgaagaaggtatcggatcgtaaagctctgttgtaagtgaagaacggctcata |
| | | gaggaaatgctatgggagtgacggtagcttaccagaaagccacggctaactacgtgccagcagccgcggtaatacgt |
| | | aggtggcaagcgttatccggaatcattgggcgtaaagggtgcgtaggtggcgtactaagtctgtagtaaaaggcaatg |
| | | gctcaaccattgtaagctatggaaactggtatgctggagtgcagaagagggcgatggaattccatgtgtagcggtaaaa |
| | | tgcgtagatatatggaggaacaccagtggcgaaggcggtcgcctggtctgtaactgacactgaggcacgaaagcgtg |
| | | gggagcaaataggattagataccctagtagtccacgccgtaaacgatgagaactaagtgttggaggaattcagtgctgc |
| | | agttaacgcaataagttctccgcctggggagtatgcacgcaagtgtgaaactcaaaggaattgacgggggcccgcaca |
| | | agcggtggagtatgtggtttaattcgaagcaacgcgaagaaccttaccaggccttgacatggaaacaaataccctagag |
| | | atagggggataattatggatcacacaggtggtgcatggttgtcgtcagctcgtgtcgtgagatgttgggttaagtcccgc |
| | | aacgagcgcaacccttgtcgcatgttaccagcatcaagttggggactcatgcgagactgccggtgacaaaccggagg |
| | | aaggtggggatgacgtcaaatcatcatgccccttatggcctgggctacacacgtactacaatggcgaccacaaagagc |
| | | agcgacttggtgacaagaagcgaatctcataaaggtcgtctcagttcggattgaagtctgcaactcgacttcatgaagtc |
| | | ggaatcgctagtaatcgcagatcagcatgctgcggtgaatacgttctcgggccttgtacacaccgcccgtcaaaccatg |
| | | ggagtcagtaatacccgaagccggtggcataaccgtaaggagtgagccgtcgaaggtaggaccgatgactggggtta |
| | | agtcgtaacaaggtatccctacgggaacgtggggatggatcacctccttt |
| Rothia_mucilaginosa | SEQ ID | acggagagtttgattctggctcaggacgaacgctggcggcgtgcttaacacatgcaagtcgaacgatgaagcctagctt |
| | NO: 327 | gctaggtggattagtggcgaacgggtgagtaatacgtgagtaacctacctttaactctgggataagcctgggaaactgg |
| | | gtctaataccggatacgaccaatctccgcatggggtgttggtggaaagcgttatgtagtggttatagatgggctcacggc |
| | | ctatcagcttgttggtgaggtaacggctcaccaaggcgacgacgggtagccggcctgagagggtgaccggccacact |
| | | gggactgagacacggcccagactcctacgggaggcagcagtggggaatattgcacaatgggcgcaagcctgatgca |
| | | gcgacgccgcgtgagggatgacggccttcgggttgtaaacctctgttagcagggaagaagagaaattgacggtacct |
| | | gcagagaaagcgccggctaactacgtgccagcagccgcggtaatacgtagggcgcgagcgttgtccggaattattgg |
| | | gcgtaaagagcttgtaggcggtttgtcgcgtctgctgtgaaaggccggggcttaactccgtgtattgcagtgggtacgg |
| | | gcagactagagtgcagtaggggagactggaattcctggtgtagcggtggaatgcgcagatatcaggaggaacaccg |
| | | atggcgaaggcaggtctctgggctgtaactgacgctgagaagcgaaagcatggggagcgaacaggattagataccct |
| | | ggtagtccatgccgtaaacgttgggcactaggtgtgggggacattccacgttaccgcgccgtagctaacgcattaagtg |
| | | ccccgcctggggagtacggccgcaaggctaaaactcaaagaaattgacgggggcccgcacaagcggcggagcatg |
| | | cggattaattcgatgcaacgcgaagaaccttaccaaggcttgacatatactggaccgcatcagagatggtgtttcccac |
| | | ggggctggtatacaggtggtgcatggttgtcgtcagctcgtgtcgtgagatgttgggttaagtcccgcaacgagcgcaa |
| | | ccctcgttctatgttgccagcacgttatggtggggactcataggagactgccggggtcaactcggaggaaggtgggga |
| | | tgacgtcaaatcatcatgcccatatgtcagggcttcacgcatgctacaatggccggtacagagggagcgatactgtga |
| | | ggtggagctaatccctaaaagccggtctcagttcggattggggtctgcaactcgaccccatgaagtcggagtcgctagt |
| | | aatcgcagatcagcaacgctgcggtgaatacgttcccgggccttgtacacaccgcccgtcaagtcacgaaagttggta |
| | | acacccaaagccggtggcctaaccatcggggagggagccgtctaaggtgggattggcgattgggactaagtcgtaa |
| | | caaggtagccgtaccggaaggtgcggctggatcacctccttt |
| Lactobacillus_ | SEQ ID | tatgagagatgatcaggctcaggatgaacgctggcggcgtgcctaatacatgcaagtcgaacgagttctgattattgaa |
| rhamnosus | NO: 328 | aggtgcttgcatcttgatttgattttgaacgagtggcggacgggtgagcaacacgtgggtaactgcccttagtgggggat |
| | | acatttggaaacagatgctaataccgcataaatccaagaaccgcatggttcttggctgaaagatggcgtaagctatcgct |
| | | tttggatggacccgcggcgtattagctagttggtgaggtaacggctcaccaaggcaatgatacgtagccgaactgaga |
| | | ggttgatcggccacattgggactgagacacggcccaaactcctacgggaggcagcagtagggaatcttccacaatgg |
| | | acgcaagtagatggagcaacgccgcgtgagtgaagaaggattcgggtcgtaaaactctgttgttggagaagaatggt |
| | | cggcagagtaactgttgtcggcgtgacggtatccaaccagaaagccacggctaactacgtgccagcagccgcggtaa |
| | | tacgtaggtggcaagcgttatccggatttattgggcgtaaagcgagcgcaggcggattttaagtctgatgtgaaagccct |
| | | cggcttaaccgaggaagtgcatcggaaactgggaaacttgagtgcagaagaggacagtggaactccatgtgtagcgg |
| | | tgaaatgcgtagatatatggaagaacaccagtggcgaaggcggctgtctggtctgtaactgacgctgaggctcgaaag |
| | | catgggtagcgaacaggattagataccctggtagtccatgccgtaaacgatgaatgctaggtgttggagggtttccgcc |
| | | cttcagtgccgcagctaacgcattaagcattccgcctggggagtacgaccgcaaggttgaaactcaaaggaattgacg |
| | | ggggcccgcacaagcggtggagcatgtggtttaattcgaagcaacgcgaagaaccttaccaggtatgacatcattga |
| | | tcacctgagagatcaggtttccccttcgggggcaaaatgacaggtggtgtatggttgtcgtcagctcgtgtcgtgagatg |
| | | agggttaagtcccgcaacgagcgcaaccatatgactagagccagcatttagttgggcactctagtaagactgccggt |
| | | gacaaaccggaggaaggtggggatgacgtcaaatcatcatgccccttatgacctgggctacacacgtgctacaatgga |
| | | tggtacaacgagttgcgagaccgcgaggtcaagctaatctcttaaagccattctcagttcggactgtaggctgcaactcg |
| | | cctacacgaagtcggaatcgctagtaatcgcggatcagcacgccgcggtgaatacgttcccgggccttgtacacaccg |
| | | cccgtcacaccatgagagtttgtaacacccgaagccggtggcgtaaccatttagggagcgagccgtctaaggtggga |
| | | caaatgattagggtgaagtcgtaacaaggtagccgtaggagaacctgcggctggatcacctcat |
| Lactobacillus_ | SEQ ID | tatgagagatgatcaggctcaggatgaacgctggcggcgtgcctaatacatgcaagtcgaacgagactgattattgaa |
| rhamnosus | NO: 329 | aggtgchgcatcttgatttaattttgaacgagtggcggacgggtgagtaacacgtgggtaacctgcccttaagtggggg |
| | | ataacatttggaaacagatgctaataccgcataaatccaagaaccgcatggttcttggctgaaagatggcgtaagctatc |
| | | gataggatggacccgcggcgtattagctagttggtgaggtaacggctcaccaaggcaatgaatacgtagccgaactg |
| | | agaggttgatcggccacattgggactgagacacggcccaaactcctacgggaggcagcagtagggaatcttccacaa |
| | | tggacgcaagtctgatggagcaacgccgcgtgagtgaagaaggctttcgggtcgtaaaactctgttgttggagaagaat |
| | | ggtcggcagagtaactgttgtcggcgtgacggtatccaaccagaaagccacggctaactacgtgccagcagccgcgg |
| | | taatacgtaggtggcaagcgttatccggatttattgggcgtaaagcgagcgcaggcggttttttaagtctgatgtgaaagc |
| | | cctcggcttaaccgaggaagtgcatcggaaactgggaaacttgagtgcagaagaggacagtggaactccatgtgtag |
| | | cggtgaaatgcgtagatatatggaagaacaccagtggcgaaggcggctgtctggtctgtaactgacgctgaggctcga |
| | | aagcatgggtagcgaacaggattagataccctggtagtccatgccgtaaacgatgaatgctaggtgttggagggtttcc |
| | | gcccttcagtgccgcagctaacgcattaagcattccgcctggggagtacgaccgcaaggttgaaactcaaaggaattg |
| | | acgggggcccgcacaagcggtggagcatgtggtttaattcgaagcaacgcgaagaaccttaccaggtcttgacatcat |
| | | tgatcacctgagagatcaggtttccccttcgggggcaaaatgacaggtggtgtatggttgtcgtcagctcgtgtcgtgag |
| | | atgttgggttaagtcccgcaacgagcgcaacccttatgactagttgccagcatttagttgggcactctagtaagactgcc |
| | | ggtgacaaaccggaggaaggtggggatgacgtcaaatcatcatgccccttatgacctgggctacacacgtgctacaat |
| | | ggatggtacaacgagttgcgagaccgcgaggtcaagctaatctcttaaagccattctcagttcggactgtaggctgcaa |
| | | ctcgcctacacgaagtcggaatcgctagtaatcgcggatcagcacgccgcggtgaatacgacccgggccagtacac |
| | | accgcccgtcacaccatgagagtttgtaacacccgaagccggtggcgtaacccttttagggagcgagccgtctaaggt |
| | | gggacaaatgattagggtgaagtcgtaacaaggtagccgtaggagaacctgcggctggatcacctcctt |
| Klebsiella_oxytoca | SEQ ID | ttgaagagtttgatcatggctcagattgaacgctggcggcaggcctaacacatgcaagtcgaacggtagcacagagag |
| | NO: 330 | cttgctctcgggtgacgagtggcggacgggtgagtaatgtctgggaaactgcccgatggagggggataactactgga |
| | | aacggtagctaataccgcataacgtcgcaagaccaaagagggggaccttagggcctatgccatcggatgtgcccag |
| | | atgggattagcttgtaggtgaggtaacggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtatgaagaaggccttcgggttgtaaagtactttcagcggggaggaagggagtgaggttaataa |
| | | ccttattcattgacgttacccgcagaagaagcaccggctaactccgtgccagcagccgcggtaatacggagggtgcaa |
| | | gcgttaatcggaattactgggcgtaaagcgcacgcaggcggtctgtcaagtcggatgtgaaatccccgggctcaacct |
| | | gggaactgcattcgaaactggcaggctggagtcttgtagaggggggtagaattccaggtgtagcggtgaaatgcgtag |
| | | agatctggaggaataccggtggcgaaggcggccccctggacaaagactgacgctcaggtgcgaaagcgtggggag |
| | | caaacaggattagataccctggtagtccacgctgtaaacgatgtcgacttggaggttgttcccttgaggagtggcttccg |
| | | gagctaacgcgttaagtcgaccgcctggggagtacggccgcaaggttaaaactcaaatgaattgacgggggcccgca |
| | | caagcggtggagcatgtggtttaattcgatgcaacgcgaagaaccttacctactcttgacatc |
| Clostridium_ | SEQ ID | attgagagatgatcctggctcaggatgaacgctggcggcgtgcttaacacatgcaagtcgagcgatgaagtttccttcg |
| perfringens | NO: 331 | ggaaacggattagcggcggacgggtgagtaacacgtgggtaacctgcctcatagagtggaatagccttccgaaagga |
| | | agattaataccgcataatgttgaaagatggcatcatcattcaaccaaaggagcaatccgctatgagatggacccgcggc |
| | | gcattagctagttggtggggtaacggcctaccaaggcgacgatgcgtagccgacctgagagggtgatcggccacatt |
| | | gggactgagacacggcccagactcctacgggaggcagcagtggggaatattgcacaatgggggaaaccctgatgca |
| | | gcaacgccgcgtgagtgatgaaggttttcggatcgtaaagctctgtcatggggaagataatgacggtacccaaggagg |
| | | aagccacggctaactacgtgccagcagccgcggtaatacgtaggtggcgagcgttatccggatttactgggcgtaaag |
| | | ggagcgtaggcggatgattaagtgggatgtgaaatacccgggctcaacttgggtgctgcattccaaactggttatctag |
| | | agtgcaggagaggagagtggaattcctagtgtagcggtgaaatgcgtagagattaggaagaacaccagtggcgaag |
| | | gcgactctctggactgtaactgacgctgaggctcgaaagcgtggggagcaaacaggattagataccctggtagtccac |
| | | gccgtaaacgatgaatactaggtgtgggggtttcaacacctccgtgccgccgctaacgcattaagtattccgcctgggg |
| | | agtacggtcgcaagattaaaactcaaaggaattgacggggacccgcacaagtagcggagcatgtggtttaattcgaag |
| | | caacgcgaagaaccttacctacacttgacatcccttgcattactcttaatcgaggaaatcccttcggggacaaggtgaca |
| | | ggtggtgcatggagtcgtcagctcgtgtcgtgagatgagggttaagtcccgcaacgagcgcaacccttgtcgttagtta |
| | | ctaccattaagttgaggactctagcgagactgcctgggttaaccaggaggaaggtggggatgacgtcaaatcatcatgc |
| | | cccttatgtgtagggctacacacgtgctacaatggctggtacagagagatgcaataccgcgaggtggagccaaactta |
| | | aaaaccagtctcagttcggattgtaggctgaaactcgcctacatgaagctggagttactagtaatcgcgaatcagaatgt |
| | | cgcggtgaatacgttcccgggtcttgtacacaccgcccgtcacaccatgagagttggcaatacccgaagtccgtgagct |
| | | aaccgcaaggaggcagcggccgaaggtagggtcagcgattggggtgaagtcgtaacaaggtagccgtaggagaac |
| | | ctgcggctggatcacctccttt |
| Streptococcus_mitis_ | SEQ ID | taatgagagtttgatcctggctcaggacgaacgctggcggcgtgcctaatacatgcaagtagaacgctgaaggaggag |
| oralis_pneumoniae | NO: 332 | cttgcttctccggatgagttgcgaacgggtgagtaacgcgtaggtaacctgcctggtagcgggggataactattggaaa |
| | | cgatagctaataccgcataacagtagatgttgcatgacatttgcttaaaaggtgcaattgcatcactaccagatggacctg |
| | | cgagtattagctagaggtggggtaacggctcaccaaggcgacgatacatagccgacctgagagggtgatcggccac |
| | | actgggactgagacacggcccagactcctacgggaggcagcagtagggaatcttcggcaatggacggaagtctgac |
| | | cgagcaacgccgcgtgagtgaagaaggttttcggatcgtaaagctctgttgtaagagaagaacgagtgtgagagtgga |
| | | aagttcacactgtgacggtatcttaccagaaagggacggctaactacgtgccagcagccgcggtaatacgtaggtccc |
| | | gagcgttgtccggatttattgggcgtaaagcgagcgcaggcggttagataagtctgaagttaaaggctgtggcttaacc |
| | | atagtacgctttggaaactgtttaacttgagtgcaagaggggagagtggaattccatgtgtagcggtgaaatgcgtagat |
| | | atatggaggaacaccggtggcgaaagcggctctctggcttgtaactgacgctgaggctcgaaagcgtggggagcaaa |
| | | caggattagataccctggtagtccacgccgtaaacgatgagtgctaggtgttagaccctttccggggtttagtgccgcag |
| | | ctaacgcattaagcactccgcctggggagtacgaccgcaaggttgaaactcaaaggaattgacgggggcccgcacaa |
| | | gcggtggagcatgtggtttaattcgaagcaacgcgaagaaccttaccaggtcttgacatccctctgaccgctctagagat |
| | | agagattccttcgggacagaggtgacaggtggtgcatggttgtcgtcagctcgtgtcgtgagatgttgggttaagtccc |
| | | gcaacgagcgcaacccctattgttagttgccatcatttagttgggcactctagcgagactgccggtaataaaccggagg |
| | | aaggtggggatgacgtcaaatcatcatgccccttatgacctgggctacacacgtgctacaatggctggtacaacgagtc |
| | | gcaagccggtgacggcaagctaatctcttaaagccagtctcagttcggattgtaggctgcaactcgcctacatgaagtc |
| | | ggaatcgctagtaatcgcggatcagcacgccgcggtgaatacgttcccgggccttgtacacaccgcccgtcacaccac |
| | | gagagtttgtaacacccgaagtcggtgaggtaaccgtaaggagccagccgcctaaggtgggatagatgattggggtg |
| | | aagtcgtaacaaggtagccgtatcggaaggtgcggctggatcacctcctt |
| Bacteroides_ovatus | SEQ ID | cgatggataggggttctgagaggaaggtcccccacattggaactgagacacggtccaaactcctacgggaggcagca |
| | NO: 333 | gtgaggaatattggtcaatgggcgagagcctgaaccagccaagtagcgtgaaggatgaaggctctatgggtcgtaaa |
| | | cttcttttatatgggaataaagttttccacgtgtggaattttgtatgtaccatatgaataaggatcggctaactccgtgccagc |
| | | agccgcggtaatacggaggatccgagcgttatccggatttattgggtttaaagggagcgtaggtggattgttaagtcagt |
| | | tgtgaaagtttgcggctcaaccgtaaaattgcagttgaaactggcagtcttgagtacagtagaggtgggcggaattcgtg |
| | | gtgtagcggtgaaatgcttagatatcacgaagaactccgattgcgaaggcagctcactagactgtcactgacactgatg |
| | | ctcgaaagtgtgggtatcaaacaggattagataccctggtagtccacacagtaaacgatgaatactcgctgtttgcgatat |
| | | acagtaagcggccaagcgaaagcattaagtattccacctggggagtacgccggcaacggtgaaactcaaaggaattg |
| | | acgggggcccgcacaagcggaggaacatgtggtttaattcgatgatacgcgaggaaccttacccgggcttaaattgca |
| | | acagaatatattggaaacagtatagccgtaaggctgttgtgaaggtgctgcatggttgtcgtcagctcgtgccgtgaggt |
| | | gtcggcttaagtgccataacgagcgcaacccttatctttagttactaacaggtcatgctgaggactctagagagactgcc |
| | | gtcgtaagatgtgaggaaggtggggatgacgtcaaatcagcacggccccttacgtccggggctacacacgtgttacaa |
| | | tggggggtacagaaggcggctacctggtgacaggatgctaatcccaaaaacctctctcagttcggatcgaagtctgca |
| | | acccgacttcgtgaagctggattcgctagtaatcgcgcatcagccatggcgcggtgaatacgttcccgggccttgtaca |
| | | caccgcccgtcaagccatgaaagccgggggtacctgaagtacgtaaccgcaaggagcgtcctagggtaaaactggt |
| | | aattggggctaagtcgtaacaaggtagccgtaccggaaggtgcggctggaacacctcctt |
| Bacteroides_ovatus | SEQ ID | atgaagagtttgatcctggctcaggatgaacgctagctacaggcttaacacatgcaagtcgaggggcagcattttagttt |
| | NO: 334 | gcttgcaaactgaagatggcgaccggcgcacgggtgagtaacacgtatccaacctgccgataactccggaatagcctt |
| | | tcgaaagaaagattaataccggatagcatacgaatatcgcatgatatttttattaaagaatttcggttatcgatggggatgc |
| | | gaccattagatgaggcggggtaacggcccaccaagactacgatggataggggttctgagaggaaggtcccccacat |
| | | tggaactgagacacggtccaaactcctacgggaggcagcagtgaggaatattggtcaatgggcgagagcctgaacca |
| | | gccaagtagcgtgaaggatgaaggctctatgggtcgtaaacttatttatatgggaataaagtatccacgtgtggaatttt |
| | | gtatgtaccatatgaataaggatcggctaactccgtgccagcagccgcggtaatacggaggatccgagcgttatccgg |
| | | atttattgggtttaaagggagcgtaggtggattgttaagtcagttgtgaaagtttgcggctcaaccgtaaaattgcagttga |
| | | aactggcagtcttgagtacagtagaggtgggcggaattcgtggtgtagcggtgaaatgcttagatatcacgaagaactc |
| | | cgattgcgaaggcagctcactagactgtcactgacactgatgctcgaaagtgtgggtatcaaacaggattagataccct |
| | | ggtagtccacacagtaaacgatgaatactcgctgtttgcgatatacagtaagcggccaagcgaaagcattaagtattcca |
| | | cctggggagtacgccggcaacggtgaaactcaaaggaattgacgggggcccgcacaagcggaggaacatgtggttt |
| | | aattcgatgatacgcgaggaaccttacccgggcttaaattgcaacagaatatattggaaacagtatagccgtaaggctgt |
| | | tgtgaaggtgctgcatggagtcgtcagctcgtgccgtgaggtgtcggcttaagtgccataacgagcgcaacccttatctt |
| | | tagttactaacaggttatgctgaggactctagagagactgccgtcgtaagatgtgaggaaggtggggatgacgtcaaat |
| | | cagcacggccccttacgtccggggctacacacgtgttacaatggggggtacagaaggcggctacctggtgacaggat |
| | | gctaatcccaaaaacctctctcagttcggatcgaagtctgcaacccgacttcgtgaagctggattcgctagtaatcgcgc |
| | | atcagccatggcgcggtgaatacgttcccgggccttgtacacaccgcccgtcaagccatgaaagccgggggtacctg |
| | | aagtacgtaaccgcaaggagcgtcctagggtaaaactggtaattggggctaagtcgtaacaaggtagccgtaccgga |
| | | aggtgcggctggaacacctcctt |
| Bacteroides_ovatus | SEQ ID | atgaagagtttgatcctggctcaggatgaacgctagctacaggcttaacacatgcaagtcgaggggcagcattttagttt |
| | NO: 335 | gatgcaaactgaagatggcgaccggcgcacgggtgagtaacacgtatccaacctgccgataactccggaatagcctt |
| | | tcgaaagaaagattaataccggatagcatacgaatatcgcatgatatttttattaaagaatttcggttatcgatggggatgc |
| | | gaccattagatgaggcggggtaacggcccaccaagactacgatggataggggttctgagaggaaggtcccccacat |
| | | tggaactgagacacggtccaaactcctacgggaggcagcagtgaggaatattggtcaatgggcgagagcctgaacca |
| | | gccaagtagcgtgaaggatgaaggctctatgggtcgtaaacttcattatatgggaataaagtatccacgtgtggaatttt |
| | | gtatgtaccatatgaataaggatcggctaactccgtgccagcagccgcggtaatacggaggatccgagcgttatccgg |
| | | atttattgggtttaaagggagcgtaggtggattgttaagtcagttgtgaaagtttgcggctcaaccgtaaaattgcagttga |
| | | aactggcagtcttgagtacagtagaggtgggcggaattcgtggtgtagcggtgaaatgcttagatatcacgaagaactc |
| | | cgattgcgaaggcagctcactagactgtcactgacactgatgctcgaaagtgtgggtatcaaacaggattagataccct |
| | | ggtagtccacacagtaaacgatgaatactcgctgtttgcgatatacagtaagcggccaagcgaaagcattaagtattcca |
| | | cctggggagtacgccggcaacggtgaaactcaaaggaattgacgggggcccgcacaagcggaggaacatgtggttt |
| | | aattcgatgatacgcgaggaaccttacccgggcttaaattgcaacagaatatattggaaacagtatagccgtaaggctgt |
| | | tgtgaaggtgctgcatggttgtcgtcagctcgtgccgtgaggtgtcggcttaagtgccataacgagcgcaacccttatctt |
| | | tagttactaacaggttatgctgaggactctagagagactgccgtcgtaagatgtgaggaaggtggggatgacgtcaaat |
| | | cagcacggcccttacgtccggggctacacacgtgttacaatggggggtacagaaggcagctacacggcgacgtgat |
| | | gctaatcccaaaaacctctctcagttcggatcgaagtctgcaacccgacttcgtgaagctggattcgctagtaatcgcgc |
| | | atcagccatggcgcggtgaatacgttcccgggccttgtacacaccgcccgtcaagccatgaaagccgggggtacctg |
| | | aagtacgtaaccgcaaggagcgtcctagggtaaaactggtaattggggctaagtcgtaacaaggtagccgtaccgga |
| | | aggtgcggctggaacacctcctt |
| Bacteroides_ovatus | SEQ ID | atgaagagtttgatcctggctcaggatgaacgctagctacaggcttaacacatgcaagtcgaggggcagcattttagttt |
| | NO: 336 | gcttgcaaactaaagatggcgaccggcgcacgggtgagtaacacgtatccaacctgccgataactcggggatagcctt |
| | | tcgaaagaaagattaatatccgatggtatattttctccgcatggtgagaatattaaagaatttcggttatcgatggggatgc |
| | | gttccattagatgaggcggggtaacggcccaccaagaccacgatggataggggttctgagaggaaggtcccccaca |
| | | ttggaactgagacacggtccaaactcctacgggaggcagcagtgaggaatattggtcaatggacgagagtctgaacc |
| | | agccaagtagcgtgaaggatgactgccctatgggttgtaaacttcttttatatgggaataaagtgttccacgtgtgggattt |
| | | tgtatgtaccatatgaataaggatcggctaactccgtgccagcagccgcggtaatacggaggatccgagcgttatccgg |
| | | atttattgggtttaaagggagcgtaggtggattgttaagtcagttgtgaaagtttgcggctcaaccgtaaaattgcagttga |
| | | aactggcagtcttgagtacagtagaggtgggcggaattcgtggtgtagcggtgaaatgcttagatatcacgaagaactc |
| | | cgattgcgaaggcagctcactagactgttactgacactgatgctcgaaagtgtgggtatcaaacaggattagataccctg |
| | | gtagtccacacagtaaacgatgaatactcgctgatgcgatatacagtaagcggccaagcgaaagcattaagtattccac |
| | | ctggggagtacgccggcaacggtgaaactcaaaggaattgacgggggcccgcacaagcggaggaacatgtggttta |
| | | attcgatgatacgcgaggaaccttacccgggcttaaattgcaacagaatatattggaaacagtatagccgtaaggctgtt |
| | | gtgaaggtgctgcatggttgtcgtcagctcgtgccgtgaggtgtcggcttaagtgccataacgagcgcaacccttatcttt |
| | | agttactaacaggtcatgctgaggactctagagagactgccgtcgtaagatgtgaggaaggtggggatgacgtcaaat |
| | | cagcacggcccttacgtccggggctacacacgtgttacaatggggggtacagaaggcggctacctggtgacaggatg |
| | | ctaatcccaaaaacctctctcagttcggatcgaagtctgcaacccgacttcgtgaagctggattcgctagtaatcgcgcat |
| | | cagccatggcgcggtgaatacgttcccgggccttgtacacaccgcccgtcaagccatgaaagccgggggtacctgaa |
| | | gtacgtaaccgcaaggagcgtcctagggtaaaactggtaattggggctaagtcgtaacaaggtagccgtaccggaag |
| | | gtgcggctggaacacctcctt |
| Bacteroides_uniformis | SEQ ID | actgagacacggtccaaactcctacgggaggcagcagtgaggaatattggtcaatggacgagagtctgaaccagcca |
| | NO: 337 | agtagcgtgaaggatgactgccctatgggttgtaaacttcttttatacgggaataaagtgaggcacgtgtgcctttttgtat |
| | | gtaccgtatgaataaggatcggctaactccgtgccagcagccgcggtaatacggaggatccgagcgttatccggattta |
| | | agggataaagggagcgtaggcggacgcttaagtcagttgtgaaagtagcggctcaaccgtaaaattgcagttgatact |
| | | gggtgtcttgagtacagtagaggcaggcggaattcgtggtgtagcggtgaaatgcttagatatcacgaagaactccgat |
| | | tgcgaaggcagcttgctggactgtaactgacgctgatgctcgaaagtgtgggtatcaaacaggattagataccctggta |
| | | gtccacacagtaaacgatgaatactcgctgtttgcgatatacagtaagcggccaagcgaaagcgttaagtattccacctg |
| | | gggagtacgccggcaacggtgaaactcaaaggaattgacgggggcccgcacaagcggaggaacatgtggtttaatt |
| | | cgatgatacgcgaggaaccttacccgggcttgaattgcaactgaatgatgtggagacatgtcagccgcaaggcagttgt |
| | | gaaggtgctgcatggttgtcgtcagctcgtgccgtgaggtgtcggcttaagtgccataacgagcgcaacccttatcgata |
| | | gttaccatcaggttatgctggggactctgtcgagactgccgtcgtaagatgtgaggaaggtggggatgacgtcaaatca |
| | | gcacggcccttacgtccggggctacacacgtgttacaatggggggtacagaaggcagctacacggcgacgtgatgct |
| | | aatcccgaaagcctctctcagttcggattggagtctgcaacccgactccatgaagctggattcgctagtaatcgcgcatc |
| | | agccacggcgcggtgaatacgttcccgggccttgtacacaccgcccgtcaagccatgaaagccgggggtacctgaa |
| | | gtgcgtaaccgcaaggagcgccctagggtaaaactggtgattggggctaagtcgtaacaaggtagccgtaccggaag |
| | | gtgcggctggaacacctcctt |
| Bacteroides_uniformis | SEQ ID | atgaagagtttgatcctggctcaggatgaacgctagctacaggcttaacacatgcaagtcgaggggcagcatgaactta |
| | NO: 338 | gcttgctaagtttgatggcgaccggcgcacgggtgagtaacacgtatccaacctgccgatgactcggggatagcctttc |
| | | gaaagaaagattaatacccgatggcatagacttccgcatggtggaactattaaagaatttcggtcatcgatggggatgc |
| | | gaccattaggttgaggcggggtaacggcccaccaagctttcgatggataggggttctgagaggaaggtcccccacat |
| | | tggaactgagacacggtccaaactcctacgggaggcagcagtgaggaatattggtcaatggacgagagtctgaacca |
| | | gccaagtagcgtgaaggatgactgccctatgggttgtaaacttcttttatacgggaataaagtgaggcacgtgtgccttttt |
| | | gtatgtaccgtatgaataaggatcggctaactccgtgccagcagccgcggtaatacggaggatccgagcgttatccgg |
| | | atttattgggtttaaagggagcgtaggcggacgcttaagtcagttgtgaaagtttgcggctcaaccgtaaaattgcagttg |
| | | atactgggtgtcttgagtacagtagaggcaggcggaattcgtggtgtagcggtgaaatgcttagatatcacgaagaact |
| | | ccgattgcgaaggcagcttgctggactgtaactgacgctgatgctcgaaagtgtgggtatcaaacaggattagataccc |
| | | tggtagtccacacagtaaacgatgaatactcgctgtttgcgatatacagtaagcggccaagcgaaagcgttaagtattcc |
| | | acctggggagtacgccggcaacggtgaaactcaaaggaattgacgggggcccgcacaagcggaggaacatgtggt |
| | | ttaattcgatgatacgcgaggaaccttacccgggcttgaattgcaactgaatgatgtggagacatgtcagccgcaaggc |
| | | agttgtgaaggtgctgcatggttgtcgtcagctcgtgccgtgaggtgtcggcttaagtgccataacgagcgcaaccctta |
| | | tcgatagttaccatcaggttatgctggggactctgtcgagactgccgtcgtaagatgtgaggaaggtggggatgacgtc |
| | | aaatcagcacggccatacgtccggggctacacacgtgttacaatggggggtacagaaggcagctacacggcgacgt |
| | | gatgctaatcccgaaagcctctctcagttcggattggagtctgcaacccgactccatgaagctggattcgctagtaatcg |
| | | cgcatcagccacggcgcggtgaatacgttcccgggccttgtacacaccgcccgtcaagccatgaaagccgggggta |
| | | cctgaagtgcgtaaccgcaaggagcgccctagggtaaaactggtgattggggctaagtcgtaacaaggtagccgtac |
| | | cggaaggtgcggctggaacacctcctt |
| Bacteroides_uniformis | SEQ ID | actgagacacggtccaaactcctacgggaggcagcagtgaggaatattggtcaatggacgagagtctgaaccagcca |
| | NO: 339 | agtagcgtgaaggatgactgccctatgggttgtaaacttcttttatacgggaataaagtgaggcacgtgtgcctttttgtat |
| | | gtaccgtatgaataaggatcggctaactccgtgccagcagccgcggtaatacggaggatccgagcgttatccggattta |
| | | agggataaagggagcgtaggcggacgcttaagtcagttgtgaaagtagcggctcaaccgtaaaattgcagttgatact |
| | | gggtgtcttgagtacagtagaggcaggcggaattcgtggtgtagcggtgaaatgcttagatatcacgaagaactccgat |
| | | tgcgaaggcagcttgctggactgtaactgacgctgatgctcgaaagtgtgggtatcaaacaggattagataccctggta |
| | | gtccacacagtaaacgatgaatactcgctgtttgcgatatacagtaagcggccaagcgaaagcgttaagtattccacctg |
| | | gggagtacgccggcaacggtgaaactcaaaggaattgacgggggcccgcacaagcggaggaacatgtggtttaatt |
| | | cgatgatacgcgaggaaccttacccgggcttgaattgcaactgaatgatgtggagacatgtcagccgcaaggcagttgt |
| | | gaaggtgctgcatggttgtcgtcagctcgtgccgtgaggtgtcggcttaagtgccataacgagcgcaacccttatcgata |
| | | gttaccatcaggttatgctggggactctgtcgagactgccgtcgtaagatgtgaggaaggtggggatgacgtcaaatca |
| | | gcacggcccttacgtccggggctacacacgtgttacaatggggggtacagaaggcagctacacggcgacgtgatgct |
| | | aatcccgaaagcctctctcagttcggattggagtctgcaacccgactccatgaagctggattcgctagtaatcgcgcatc |
| | | agccacggcgcggtgaatacgttcccgggccttgtacacaccgcccgtcaagccatgaaagccgggggtacctgaa |
| | | gtgcgtaaccgcaaggagcgccctagggtaaaactggtgattggggctaagtcgtaacaaggtagccgtaccggaag |
| | | gtgcggctggaacacctcctt |
| Bacteroides_uniformis | SEQ ID | atgaagagtttgatcctggctcaggatgaacgctagctacaggcttaacacatgcaagtcgaggggcagcatgaactta |
| | NO: 340 | gcttgctaagtagatggcgaccggcgcacgggtgagtaacacgtatccaacctgccgatgactcggggatagcctttc |
| | | gaaagaaagattaatacccgatggcatagacttccgcatggtagaactattaaagaatttcggtcatcgatggggatgc |
| | | gttccattaggagttggcggggtaacggcccaccaagccttcgatggataggggttctgagaggaaggtcccccacat |
| | | tggaactgagacacggtccaaactcctacgggaggcagcagtgaggaatattggtcaatggacgagagtctgaacca |
| | | gccaagtagcgtgaaggatgactgccctatgggttgtaaacttatttatacgggaataaagtgaggcacgtgtgccattt |
| | | gtatgtaccgtatgaataaggatcggctaactccgtgccagcagccgcggtaatacggaggatccgagcgttatccgg |
| | | atttattgggtttaaagggagcgtaggcggacgcttaagtcagttgtgaaagtttgcggctcaaccgtaaaattgcagttg |
| | | atactgggtgtcttgagtacagtagaggcaggcggaattcgtggtgtagcggtgaaatgcttagatatcacgaagaact |
| | | ccgattgcgaaggcagcttgctggactgtaactgacgctgatgctcgaaagtgtgggtatcaaacaggattagataccc |
| | | tggtagtccacacagtaaacgatgaatactcgctgtttgcgatatacagtaagcggccaagcgaaagcgttaagtattcc |
| | | acctggggagtacgccggcaacggtgaaactcaaaggaattgacgggggcccgcacaagcggaggaacatgtggt |
| | | ttaattcgatgatacgcgaggaaccttacccgggcttgaattgcaactgaatgatgtggagacatgtcagccgcaaggc |
| | | agttgtgaaggtgctgcatggttgtcgtcagctcgtgccgtgaggtgtcggcttaagtgccataacgagcgcaaccctta |
| | | tcgatagttaccatcaggttatgctggggactctgtcgagactgccgtcgtaagatgtgaggaaggtggggatgacgtc |
| | | aaatcagcacggcccttacgtccggggctacacacgtgttacaatggggggtacagaaggcagctacacggcgacgt |
| | | gatgctaatcccgaaagcctctacagttcggattggagtctgcaacccgactccatgaagctggattcgctagtaatcg |
| | | cgcatcagccacggcgcggtgaatacgacccgggccttgtacacaccgcccgtcaagccatgaaagccgggggta |
| | | cctgaagtgcgtaaccgcaaggagcgccctagggtaaaactggtgattggggctaagtcgtaacaaggtagccgtac |
| | | cggaaggtgcggctggaacacctcctt |
| Bacteroides_uniformis | SEQ ID | atgaagagtagatcctggctcaggatgaacgctagctacaggcttaacacatgcaagtcgaggggcagcatgaactta |
| | NO: 341 | gcttgctaagtagatggcgaccggcgcacgggtgagtaacacgtatccaacctgccgatgactcggggatagcctttc |
| | | gaaagaaagattaatacccgatggcatagacttccgcatggtggaactattaaagaatttcggtcatcgatggggatgc |
| | | gttccattaggagttggcggggtaacggcccaccaagccttcgatggataggggttctgagaggaaggtcccccacat |
| | | tggaactgagacacggtccaaactcctacgggaggcagcagtgaggaatattggtcaatggacgagagtctgaacca |
| | | gccaagtagcgtgaaggatgactgccctatgggagtaaacttctatatacgggaataaagtgaggcacgtgtgccttat |
| | | gtatgtaccgtatgaataaggatcggctaactccgtgccagcagccgcggtaatacggaggatccgagcgttatccgg |
| | | atttattgggtttaaagggagcgtaggcggacgcttaagtcagttgtgaaagtttgcggctcaaccgtaaaattgcagttg |
| | | atactgggtgtcttgagtacagtagaggcaggcggaattcgtggtgtagcggtgaaatgcttagatatcacgaagaact |
| | | ccgattgcgaaggcagcttgctggactgtaactgacgctgatgctcgaaagtgtgggtatcaaacaggattagataccc |
| | | tggtagtccacacagtaaacgatgaatactcgctgtttgcgatatacagtaagcggccaagcgaaagcgttaagtattcc |
| | | acctggggagtacgccggcaacggtgaaactcaaaggaattgacgggggcccgcacaagcggaggaacatgtggt |
| | | ttaattcgatgatacgcgaggaaccttacccgggcttgaattgcaactgaatgatgtggagacatgtcagccgcaaggc |
| | | agagtgaaggtgctgcatggttgtcgtcagctcgtgccgtgaggtgtcggcttaagtgccataacgagcgcaaccctta |
| | | tcgatagttaccatcaggttatgctggggactctgtcgagactgccgtcgtaagatgtgaggaaggtggggatgacgtc |
| | | aaatcagcacggcccttacgtccggggctacacacgtgttacaatggggggtacagaaggcagctacacggcgacgt |
| | | gatgctaatcccgaaagcctctctcagttcggattggagtctgcaacccgactccatgaagctggattcgctagtaatcg |
| | | cgcatcagccacggcgcggtgaatacgttcccgggccttgtacacaccgcccgtcaagccatgaaagccgggggta |
| | | cctgaagtgcgtaaccgcaaggagcgccctagggtaaaactggtgattggggctaagtcgtaacaaggtagccgtac |
| | | cggaaggtgcggctggaacacctcctt |
| Bacteroides_uniformis | SEQ ID | atgaagagtagatcctggctcaggatgaacgctagctacaggcttaacacatgcaagtcgaggggcagcatgaactta |
| | NO: 342 | gcttgctaagtagatggcgaccggcgcacgggtgagtaacacgtatccaacctgccgatgactcggggatagcctttc |
| | | gaaagaaagattaatacccgatggcatagttcttccgcatggtagaactattaaagaatttcggtcatcgatggggatgc |
| | | gttccattaggttgttggcggggtaacggcccaccaagccttcgatggataggggttctgagaggaaggtcccccacat |
| | | tggaactgagacacggtccaaactcctacgggaggcagcagtgaggaatattggtcaatggacgagagtctgaacca |
| | | gccaagtagcgtgaaggatgactgccctatgggttgtaaacttcttttatacgggaataaagtgaggcacgtgtgccttttt |
| | | gtatgtaccgtatgaataaggatcggctaactccgtgccagcagccgcggtaatacggaggatccgagcgttatccgg |
| | | atttattgggtttaaagggagcgtaggcggacgcttaagtcagttgtgaaagtttgcggctcaaccgtaaaattgcagttg |
| | | atactgggtgtcttgagtacagtagaggcaggcggaattcgtggtgtagcggtgaaatgcttagatatcacgaagaact |
| | | ccgattgcgaaggcagcttgctggactgtaactgacgctgatgctcgaaagtgtgggtatcaaacaggattagataccc |
| | | tggtagtccacacagtaaacgatgaatactcgctgtttgcgatatacagtaagcggccaagcgaaagcgttaagtattcc |
| | | acctggggagtacgccggcaacggtgaaactcaaaggaattgacgggggcccgcacaagcggaggaacatgtggt |
| | | ttaattcgatgatacgcgaggaaccttacccgggcttgaattgcaactgaatgatgtggagacatgtcagccgcaaggc |
| | | agttgtgaaggtgctgcatggttgtcgtcagctcgtgccgtgaggtgtcggcttaagtgccataacgagcgcaaccctta |
| | | tcgatagttaccatcaggttatgctggggactctgtcgagactgccgtcgtaagatgtgaggaaggtggggatgacgtc |
| | | aaaatcagcacggcccttacgtccggggctaccacgtgttacaatggggggtacagaaggcagctacacggcgacgt |
| | | gatgctaatcccgaaagcctctctcagttcggattggagtctgcaacccgactccatgaagctggattcgctagtaatcg |
| | | cgcatcagccacggcgcggtgaatacgttcccgggccttgtacacaccgcccgtcaagccatgaaagccgggggta |
| | | cctgaagtgcgtaaccgcaaggagcgccctagggtaaaactggtgattggggctaagtcgtaacaaggtagccgtac |
| | | cggaaggtgcggctggaacacctcctt |
| Bacteroides_uniformis | SEQ ID | atgaagagtttgatcctggctcaggatgaacgctagctacaggcttaacacatgcaagtcgaggggcagcatgaactta |
| | NO: 343 | gcttgctaagtttgatggcgaccggcgcacgggtgagtaacacgtatccaacctgccgatgactcggggatagcctttc |
| | | gaaagaaagattaatacccgatggcatagttcttccgcatggtagaactattaaagaatttcggtcatcgatggggatgc |
| | | gttccattaggttgttggcggggtaacggcccaccaagccttcgatggataggggactgagaggaaggtcccccacat |
| | | tggaactgagacacggtccaaactcctacgggaggcagcagtgaggaatattggtcaatggacgagagtctgaacca |
| | | gccaagtagcgtgaaggatgactgccctatgggttgtaaacttcttttatacgggaataaagtgaggcacgtgtgccttttt |
| | | gtatgtaccgtatgaataaggatcggctaactccgtgccagcagccgcggtaatacggaggatccgagcgttatccgg |
| | | atttattgggtttaaagggagcgtaggcggacgcttaagtcagttgtgaaagtttgcggctcaaccgtaaaattgcagttg |
| | | atactgggtgtcttgagtacagtagaggcaggcggaattcgtggtgtagcggtgaaatgcttagatatcacgaagaact |
| | | ccgattgcgaaggcagcttgctggactgtaactgacgctgatgctcgaaagtgtgggtatcaaacaggattagataccc |
| | | tggtagtccacacagtaaacgatgaatactcgctgtttgcgatatacagtaagcggccaagcgaaagcgttaagtattcc |
| | | acctggggagtacgccggcaacggtgaaactcaaaggaattgacgggggcccgcacaagcggaggaacatgtggt |
| | | ttaattcgatgatacgcgaggaaccttacccgggcttgaattgcaactgaatgatgtggagacatgtcagccgcaaggc |
| | | agttgtgaaggtgctgcatggttgtcgtcagctcgtgccgtgaggtgtcggcttaagtgccataacgagcgcaaccctta |
| | | tcgatagttaccatcaggttatgctggggactctgtcgagactgccgtcgtaagatgtgaggaaggtggggatgacgtc |
| | | aaatcagcacggccatacgtccggggctacacacgtgttacaatggggggtacagaaggcagctacacggcgacgt |
| | | gatgctaatcccgaaagcctctctcagttcggattggagtctgcaacccgactccatgaagctggattcgctagtaatcg |
| | | cgcatcagccacggcgcggtgaatacgttcccgggccttgtacacaccgcccgtcaagccatgaaagccgggggta |
| | | cctgaagtgcgtaaccgcaaggagcgccctagggtaaaactggtgattggggctaagtcgtaacaaggtagccgtac |
| | | cggaaggtgcggctggaacacctcctt |
| Bacteroides_vulgatus | SEQ ID | tttgcatgtactttatgaataaggatcggctaactccgtgccagcagccgcggtaatacggaggatccgagcgttatccg |
| | NO: 344 | gatttattgggtttaaagggagcgtagatggatgtttaagtcagttgtgaaagtttgcggctcaaccgtaaaattgcagttg |
| | | atactggatatcttgagtgcagttgaggcaggcggaattcgtggtgtagcggtgaaatgcttagatatcacgaagaactc |
| | | cgattgcgaaggcagcctgctaagctgcaactgacattgaggctcgaaagtgtgggtatcaaacaggattagataccct |
| | | ggtagtccacacggtaaacgatgaatactcgctgtttgcgatatacggcaagcggccaagcgaaagcgttaagtattcc |
| | | acctggggagtacgccggcaacggtgaaactcaaaggaattgacgggggcccgcacaagcggaggaacatgtggt |
| | | ttaattcgatgatacgcgaggaaccttacccgggcttaaattgcagatgaattacggtgaaagccgtaagccgcaaggc |
| | | atctgtgaaggtgctgcatggttgtcgtcagctcgtgccgtgaggtgtcggcttaagtgccataacgagcgcaacccttg |
| | | ttgtcagttactaacaggttctgctgaggactctgacaagactgccatcgtaagatgtgaggaaggtggggatgacgtca |
| | | aatcagcacggcccttacgtccggggctacacacgtgttacaatggggggtacagagggccgctaccacgcgagtg |
| | | gatgccaatcccaaaaacctctctcagttcggactggagtctgcaacccgactccacgaagctggattcgctagtaatc |
| | | gcgcatcagccacggcgcggtgaatacgttcccgggccttgtacacaccgcccgtcaagccatgggagccgggggt |
| | | acctgaagtgcgtaaccgcgaggagcgccctagggtaaaactggtgactggggctaagtcgtaacaaggtagccgta |
| | | ccggaaggtgcggctggaacacctccttt |
| Bacteroides_vulgatus | SEQ ID | atgaagagtttgatcctggctcaggatgaacgctagctacaggcttaacacatgcaagtcgaggggcagcatggtctta |
| | NO: 345 | gcttgctaaggccgatggcgaccggcgcacgggtgagtaacacgtatccaacctgccgtctactcttggacagccttct |
| | | gaaaggaagattaatacaagatggcatcatgagttcacatgttcacatgattaaaggtattccggtagacgatggggatg |
| | | cgaccattagatagtaggcggggtaacggcccacctagttttcgatggataggggttctgagaggaaggtcccccac |
| | | attggaactgagacacggtccaaactcctacgggaggcagcagtgaggaatattggtcaatgggcgagagcctgaac |
| | | cagccaagtagcgtgaaggatgactgccctatgggttgtaaacttcttttataaaggaataaagtcgggtatggataccc |
| | | gtttgcatgtactttatgaataaggatcggctaactccgtgccagcagccgcggtaatacggaggatccgagcgttatcc |
| | | ggatttattgggtttaaagggagcgtagatggatgtttaagtcagttgtgaaagtttgcggctcaaccgtaaaattgcagtt |
| | | gatactggatatcttgagtgcagttgaggcaggcggaattcgtggtgtagcggtgaaatgcttagatatcacgaagaact |
| | | ccgattgcgaaggcagcctgctaagctgcaactgacattgaggctcgaaagtgtgggtatcaaacaggattagatacc |
| | | ctggtagtccacacggtaaacgatgaatactcgctgtagcgatatacggcaagcggccaagcgaaagcgttaagtatt |
| | | ccacctggggagtacgccggcaacggtgaaactcaaaggaattgacgggggcccgcacaagcggaggaacatgtg |
| | | gataattcgatgatacgcgaggaaccttacccgggcttaaattgcagatgaattacggtgaaagccgtaagccgcaag |
| | | gcatctgtgaaggtgctgcatggttgtcgtcagctcgtgccgtgaggtgtcggcttaagtgccataacgagcgcaaccc |
| | | ttgttgtcagttactaacaggttctgctgaggactctgacaagactgccatcgtaagatgtgaggaaggtggggatgacg |
| | | tcaaatcagcacggcccttacgtccggggctacacacgtgttacaatggggggtacagagggccgctaccacgcgag |
| | | tggatgccaatcccaaaaacctctctcagttcggactggagtctgcaacccgactccacgaagctggattcgctagtaat |
| | | cgcgcatcagccacggcgcggtgaatacgttcccgggccttgtacacaccgcccgtcaagccatgggagccggggg |
| | | tacctgaagtgcgtaaccgcgaggagcgccctagggtaaaactggtgactggggctaagtcgtaacaaggtagccgt |
| | | accggaaggtgcggctggaacacctcctt |
| Bacteroides_vulgatus | SEQ ID | atgaagagtttgatcctggctcaggatgaacgctagctacaggcttaacacatgcaagtcgaggggcagcatggtctta |
| | NO: 346 | gcttgctaaggccgatggcgaccggcgcacgggtgagtaacacgtatccaacctgccgtctactcttggacagccact |
| | | gaaaggaagattaatacaagatggcatcatgagtccgcatgttcacatgattaaaggtattccggtaggcgatggggat |
| | | gcgttccattagatagtaggcggggtaacggcccacctagtcttcgatggataggggttctgagaggaaggtccccca |
| | | cattggaactgagacacggtccaaactcctacgggaggcagcagtgaggaatattggtcaatgggcgagagcctgaa |
| | | ccagccaagtagcgtgaaggatgactgccctatgggttgtaaacttcttttataaaggaataaagtcgggtatgtataccc |
| | | gtttgcatgtactttatgaataaggatcggctaactccgtgccagcagccgcggtaatacggaggatccgagcgttatcc |
| | | ggatttattgggtttaaagggagcgtagatggatgtttaagtcagttgtgaaagtttgcggctcaaccgtaaaattgcagtt |
| | | gatactggatatcttgagtgcagttgaggcaggcggaattcgtggtgtagcggtgaaatgcttagatatcacgaagaact |
| | | ccgattgcgaaggcagcctgctaagctgcaactgacattgaggctcgaaagtgtgggtatcaaacaggattagatacc |
| | | ctggtagtccacacggtaaacgatgaatactcgctgtttgcgatatacagcaagcggccaagcgaaagcgttaagtatt |
| | | ccacctggggagtacgccggcaacggtgaaactcaaaggaattgacgggggcccgcacaagcggaggaacatgtg |
| | | gataattcgatgatacgcgaggaaccttacccgggcttaaattgcagatgaattacggtgaaagccgtaagccgcaag |
| | | gcatctgtgaaggtgctgcatggagtcgtcagctcgtgccgtgaggtgtcggcttaagtgccataacgagcgcaaccc |
| | | ttgttgtcagttactaacaggttctgctgaggactctgacaagactgccatcgtaagatgtgaggaaggtggggatgacg |
| | | atcaaatcagcacggcccttacgtccggggctacacacgtgttacaatggggggtacagagggccgctaccacgcgg |
| | | tggatgccaatcccaaaaacctctctcagttcggactggagtctgcaacccgactccacgaagctggattcgctagtaat |
| | | cgcgcatcagccacggcgcggtgaatacgttcccgggccttgtacacaccgcccgtcaagccatgggagccggggg |
| | | tacctgaagtgcgtaaccgcgaggagcgccctagggtaaaactggtgactggggctaagtcgtaacaaggtagccgt |
| | | accggaaggtgcggctggaacacctcctt |
| Bacteroides_vulgatus | SEQ ID | atgaagagtttgatcctggctcaggatgaacgctagctacaggcttaacacatgcaagtcgaggggcagcatggtctta |
| | NO: 347 | gcttgctaaggccgatggcgaccggcgcacgggtgagtaacacgtatccaacctgccgtctactcttggacagccttct |
| | | gaaaggaagattaatacaagatggcatcatgagtccgcatgttcacatgattaaaggtattccggtagacgatggggat |
| | | gcgaccattagatagtaggcggggtaacggcccacctagtcttcgatggataggggttctgagaggaaggtccccca |
| | | cattggaactgagacacggtccaaactcctacgggaggcagcagtgaggaatattggtcaatgggcgcaggcctgaa |
| | | ccagccaagtagcgtgaaggatgactgccctatgggttgtaaacttcttttataaaggaataaagtcgggtatgcataccc |
| | | gtttgcatgtactttatgaataaggatcggctaactccgtgccagcagccgcggtaatacggaggatccgagcgttatcc |
| | | ggatttattgggtttaaagggagcgtagatggatgtttaagtcagttgtgaaagtttgcggctcaaccgtaaaattgcagtt |
| | | gatactggatatcttgagtgcagttgaggcaggcggaattcgtggtgtagcggtgaaatgcttagatatcacgaagaact |
| | | ccgattgcgaaggcagcctgctaagctgcaactgacattgaggctcgaaagtgtgggtatcaaacaggattagatacc |
| | | ctggtagtccacacggtaaacgatgaatactcgctgtttgcgatatacagcaagcggccaagcgaaagcgttaagtatt |
| | | ccacctggggagtacgccggcaacggtgaaactcaaaggaattgacgggggcccgcacaagcggaggaacatgtg |
| | | gataattcgatgatacgcgaggaaccttacccgggcttaaattgcagatgaattttcggtgaaagccgtaagccgcaag |
| | | gcatctgtgaaggtgctgcatggttgtcgtcagctcgtgccgtgaggtgtcggcttaagtgccataacgagcgcaaccc |
| | | ttgttgtcagttactaacaggttccgctgaggactctgacaagactgccatcgtaagatgtgaggaaggtggggatgac |
| | | gtcaaatcagcacggcccttacgtccggggctacacacgtgttacaatggggggtacagagggtcgctaccacgcga |
| | | gtggatgccaatccccaaaacctctctcagttcggactggagtctgcaacccgactccacgaagctggattcgctagta |
| | | atcgcgcatcagccacggcgcggtgaatacgttcccgggccttgtacacaccgcccgtcaagccatgggagccggg |
| | | ggtacctgaagtgcgtaaccgcgaggagcgccctagggtaaaactggtgactggggctaagtcgtaacaaggtagcc |
| | | gtaccggaaggtgcggctggaacacctcctt |
| Bacteroides_vulgatus | SEQ ID | atgaagagtttgatcctggctcaggatgaacgctagctacaggcttaacacatgcaagtcgaggggcagcatggtctta |
| | NO: 348 | gcttgctaaggccgatggcgaccggcgcacgggtgagtaacacgtatccaacctgccgtctactcdggacagccact |
| | | gaaaggaagattaatacaagatggcatcatgagtccgcatgttcacatgattaaaggtattccggtagacgatggggat |
| | | gcgttccattagatagtaggcggggtaacggcccacctagtcttcgatggataggggttctgagaggaaggtccccca |
| | | cattggaactgagacacggtccaaactcctacgggaggcagcagtgaggaatattggtcaatgggcgagagcctgaa |
| | | ccagccaagtagcgtgaaggatgactgccctatgggttgtaaacttcttttataaaggaataaagtcgggtatggatacc |
| | | cgtttgcatgtactttatgaataaggatcggctaactccgtgccagcagccgcggtaatacggaggatccgagcgttatc |
| | | cggatttattgggtttaaagggagcgtagatggatgtttaagtcagttgtgaaagtttgcggctcaaccgtaaaattgcagt |
| | | tgatactggatatcttgagtgcagttgaggcaggcggaattcgtggtgtagcggtgaaatgatagatatcacgaagaac |
| | | tccgattgcgaaggcagcctgctaagctgcaactgacattgaggctcgaaagtgtgggtatcaaacaggattagatacc |
| | | ctggtagtccacacggtaaacgatgaatactcgctgtttgcgatatacagcaagcggccaagcgaaagcgttaagtatt |
| | | ccacctggggagtacgccggcaacggtgaaactcaaaggaattgacgggggcccgcacaagcggaggaacatgtg |
| | | gataattcgatgatacgcgaggaaccttacccgggcttaaattgcagatgaattttcggtgaaagccgtaagccgcaag |
| | | gcatctgtgaaggtgctgcatggttgtcgtcagctcgtgccgtgaggtgtcggcttaagtgccataacgagcgcaaccc |
| | | ttgttgtcagttactaacaggttctgctgaggactctgacaagactgccatcgtaagatgtgaggaaggtggggatgacg |
| | | tcaaatcagcacggcccttacgtccggggctacacacgtgttacaatggggggtacagagggccgctaccacgcgag |
| | | tggatgccaatcccaaaaacctctctcagttcggactggagtctgcaacccgactccacgaagctggattcgctagtaat |
| | | cgcgcatcagccacggcgcggtgaatacgttcccgggccttgtacacaccgcccgtcaagccatgggagccggggg |
| | | tacctgaagtgcgtaaccgcgaggagcgccctagggtaaaactggtgactggggctaagtcgtaacaaggtagccgt |
| | | accggaaggtgcggctggaacacctcctt |
| Bacteroides_vulgatus | SEQ ID | ccgtttgcatgtactttatgaataaggatcggctaactccgtgccagcagccgcggtaatacggaggatccgagcgttat |
| | NO: 349 | ccggatttattgggtttaaagggagcgtagatggatgtttaagtcagttgtgaaagtttgcggctcaaccgtaaaattgca |
| | | gagatactggatatatgagtgcagttgaggcaggcggaattcgtggtgtagcggtgaaatgcttagatatcacgaaga |
| | | actccgattgcgaaggcagcctgctaagctgcaactgacattgaggctcgaaagtgtgggtatcaaacaggattagata |
| | | ccctggtagtccacacggtaaacgatgaatactcgctgtttgcgatatacagcaagcggccaagcgaaagcgttaagta |
| | | ttccacctggggagtacgccggcaacggtgaaactcaaaggaattgacgggggcccgcacaagcggaggaacatgt |
| | | ggtttaattcgatgatacgcgaggaaccttacccgggcttaaattgcagatgaattacggtgaaagccgtaagccgcaa |
| | | ggcatctgtgaaggtgctgcatggttgtcgtcagctcgtgccgtgaggtgtcggcttaagtgccataacgagcgcaacc |
| | | cttgttgtcagttactaacaggttctgctgaggactctgacaagactgccatcgtaagatgtgaggaaggtggggatgac |
| | | gtcaaatcagcacggcccttacgtccggggctacacacgtgttacaatggggggtacagagggccgctaccacgcga |
| | | gtggatgccaatccccaaaacctctctcagttcggactggagtctgcaacccgactccacgaagctggattcgctagta |
| | | atcgcgcatcagccacggcgcggtgaatacgttcccgggccttgtacacaccgcccgtcaagccatgggagccggg |
| | | ggtacctgaagtgcgtaaccgcgaggagcgccctagggtaaaactggtgactggggctaagtcgtaacaaggtagcc |
| | | gtaccggaaggtgcggctggaacacctccttt |
| Eubacterium_sp_3_1_ | SEQ ID | atggagagtagatcctggctcaggatgaacgctggcggcatgcctaatacatgcaagtcgaacgaagtcataggaag |
| 31 | NO: 350 | cttgcttccaaagagacttagtggcgaacgggtgagtaacacgtaggtaacctgcccatgtgcccgggataactgctgg |
| | | aaacggtagctaaaaccggataggtatgagggaggcatcttcctcatattaaagcaccttcgggtgtgaacatggatgg |
| | | acctgcggcgcattagctggttggtgaggtaacggcccaccaaggcgatgatgcgtagccgacctgagagggtgaac |
| | | ggccacattgggactgagacacggcccaaactcctacgggaggcagcagtagggaattttcgtcaatggggggaac |
| | | cctgaacgagcaatgccgcgtgtgtgaagaaggtcttcggatcgtaaagcactgagtaagtgaagaatgccacataga |
| | | ggaaatgctatgtgggtgacggtagcttaccagaaagccacggctaactacgtgccagcagccgcggtaatacgtag |
| | | gtggcaagcgttatccggaatcattgggcgtaaagggtgcgtaggtggcacgataagtctgaagtaaaaggcaacag |
| | | ctcaactgagtatgctttggaaactgtcgagctagagtgcagaagagggcgatggaattccatgtgtagcggtaaaatg |
| | | cgtagatatatggaggaacaccagtggcgaaggcggtcgcctggtctgtaactgacactgatgcacgaaagcgtggg |
| | | gagcaaataggattagataccctagtagtccacgccgtaaacgatgagaactaagtgttggagagattcagtgctgcag |
| | | ttaacgcaataagttctccgcctggggagtatgcacgcaagtgtgaaactcaaaggaattgacgggggcccgcacaag |
| | | cggtggagtatgtggtttaattcgaagcaacgcgaagaaccttaccaggccttgacatggatataaatgttctagagata |
| | | gaaagatagctatatatcacacaggtggtgcatggttgtcgtcagctcgtgtcgtgagatgttgggttaagtcccgcaac |
| | | gagcgcaacccagtcttctgttaccagcattaggttggggactcaggagagactgccggtgacaaaccggaggaagg |
| | | tggggatgacgtcaaatcatcatgccccttatggcctgggctacacacgtactacaatggcgcctacaaagagcagcg |
| | | acaccgcgaggtggagcgaatctcataaagggcgtctcagttcggattgaagtctgcaactcgacttcatgaagtcgga |
| | | atcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagt |
| | | cgtaacaaggtatccctacgggaacgtggggatggatcacctccttt |
| Eubacterium_sp_3_1_ | SEQ ID | atggagagtttgatcctggctcaggatgaacgctggcggcatgcctaatacatgcaagtcgaacgaagtctttaggaag |
| 31 | NO: 351 | cttgcttccaaagagacttagtggcgaacgggtgagtaacacgtaggtaacctgcccatgtgcccgggataactgctgg |
| | | aaacggtagctaaaaccggataggtatgagggaggcatcttcctcatattaaagcaccttcgggtgtgaacatggatgg |
| | | acctgcggcgcattagctggttggtgaggtaacggcccaccaaggcgatgatgcgtagccgacctgagagggtgaac |
| | | ggccacattgggactgagacacggcccaaactcctacgggaggcagcagtagggaattttcgtcaatggggggaac |
| | | cctgaacgagcaatgccgcgtgtgtgaagaaggtcttcggatcgtaaagcactgagtaagtgaagaatgccacataga |
| | | ggaaatgctatgtgggtgacggtagcttaccagaaagccacggctaactacgtgccagcagccgcggtaatacgtag |
| | | gtggcaagcgttatccggaatcattgggcgtaaagggtgcgtaggtggcacgataagtctgaagtaaaaggcaacag |
| | | ctcaactgagtatgctttggaaactgtcgagctagagtgcagaagagggcgatggaattccatgtgtagcggtaaaatg |
| | | cgtagatatatggaggaacaccagtggcgaaggcggtcgcctggtctgtaactgacactgatgcacgaaagcgtggg |
| | | gagcaaataggattagataccctagtagtccacgccgtaaacgatgagaactaagtgttggagagattcagtgctgcag |
| | | ttaacgcaataagttctccgcctggggagtatgcacgcaagtgtgaaactcaaaggaattgacgggggcccgcacaag |
| | | cggtggagtatgtggtttaattcgaagcaacgcgaagaaccttaccaggccttgacatggatataaatgttctagagata |
| | | gaaagatagctatatatcacacaggtggtgcatggttgtcgtcagctcgtgtcgtgagatgttgggttaagtcccgcaac |
| | | gagcgcaacccttgtcttctgttaccagcattaggttggggactcaggagagactgccggtgacaaaccggaggaagg |
| | | tggggatgacgtcaaatcatcatgccccttatggcctgggctacacacgtactacaatggcgcctacaaagagcagcg |
| | | acaccgcgaggtggagcgaatctcataaagggcgtctcagttcggattgaagtctgcaactcgacttcatgaagtcgga |
| | | atcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagt |
| | | cgtaacaaggtatccctacgggaacgtggggatggatcacctccttt |
| Eubacterium_sp_3_1_ | SEQ ID | atggagagatgatcctggctcaggatgaacgctggcggcatgcctaatacatgcaagtcgaacgaagtcataggaag |
| 31 | NO: 352 | cttgcttccaaagagacttagtggcgaacgggtgagtaacacgtaggtaacctgcccatgtgcccgggataactgctgg |
| | | aaacggtagctaaaaccggataggtatgagggaggcatcttcctcatattaaagcaccttcgggtgtgaacatggatgg |
| | | acctgcggcgcattagctggttggtgaggtaacggcccaccaaggcgatgatgcgtagccgacctgagagggtgaac |
| | | ggccacattgggactgagacacggcccaaactcctacgggaggcagcagtagggaattttcgtcaatggggggaac |
| | | cctgaacgagcaatgccgcgtgtgtgaagaaggtcttcggatcgtaaagcactgagtaagtgaagaatgccatataga |
| | | ggaaatgctatgtgggtgacggtagcttaccagaaagccacggctaactacgtgccagcagccgcggtaatacgtag |
| | | gtggcaagcgttatccggaatcattgggcgtaaagggtgcgtaggtggcacgataagtctgaagtaaaaggcaacag |
| | | ctcaactgagtatgctttggaaactgtcgagctagagtgcagaagagggcgatggaattccatgtgtagcggtaaaatg |
| | | cgtagatatatggaggaacaccagtggcgaaggcggtcgcctggtctgtaactgacactgatgcacgaaagcgtggg |
| | | gagcaaataggattagataccctagtagtccacgccgtaaacgatgagaactaagtgttggagagattcagtgctgcag |
| | | ttaacgcaataagttctccgcctggggagtatgcacgcaagtgtgaaactcaaaggaattgacgggggcccgcacaag |
| | | cggtggagtatgtggtttaattcgaagcaacgcgaagaaccttaccaggccttgacatggatataaatgttctagagata |
| | | gaaagatagctatatatcacacaggtggtgcatggagtcgtcagctcgtgtcgtgagatgagggttaagtcccgcaac |
| | | gagcgcaacccttgtcttctgttaccagcattaggttggggactcaggagagactgccggtgacaaaccggaggaagg |
| | | tggggatgacgtcaaatcatcatgccccttatggcctgggctacacacgtactacaatggcgcctacaaagagcagcg |
| | | acaccgcgaggtgaagcgaatctcataaagggcgtctcagttcggattgaagtctgcaactcgacttcatgaagtcgga |
| | | atcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagt |
| | | cgtaacaaggtatccctacgggaacgtggggatggatcacctccttt |
| Eubacterium_sp_3_1_ | SEQ ID | atggagagtagatcctggctcaggatgaacgctggcggcatgcctaatacatgcaagtcgaacgaagtcataggaag |
| 31 | NO: 353 | cagcaccaaagagacttagtggcgaacgggtgagtaacacgtaggtaacctgcccatgtgcccgggataactgctgg |
| | | aaacggtagctaaaaccggataggtatgagggaggcatcttcctcatattaaagcaccacgggtgtgaacatggatgg |
| | | acctgcggcgcattagctggttggtgaggtaacggcccaccaaggcgatgatgcgtagccgacctgagagggtgaac |
| | | ggccacattgggactgagacacggcccaaactcctacgggaggcagcagtagggaattacgtcaatggggggaac |
| | | cctgaacgagcaatgccgcgtgtgtgaagaaggtcttcggatcgtaaagcactgttgtaagtgaagaatgccacataga |
| | | ggaaatgctatgtgggtgacggtagcttaccagaaagccacggctaactacgtgccagcagccgcggtaatacgtag |
| | | gtggcaagcgttatccggaatcattgggcgtaaagggtgcgtaggtggcacgataagtctgaagtaaaaggcaacag |
| | | ctcaactgagtatgctttggaaactgtcgagctagagtgcagaagagggcgatggaattccatgtgtagcggtaaaatg |
| | | cgtagatatatggaggaacaccagtggcgaaggcggtcgcctggtctgtaactgacactgatgcacgaaagcgtggg |
| | | gagcaaataggattagataccctagtagtccacgccgtaaacgatgagaactaagtgttggagagattcagtgctgcag |
| | | ttaacgcaataagttctccgcctggggagtatgcacgcaagtgtgaaactcaaaggaattgacgggggcccgcacaag |
| | | cggtggagtatgtggtttaattcgaagcaacgcgaagaaccttaccaggccttgacatggatataaatgttctagagata |
| | | gaaagatagctatatatcacacaggtggtgcatggttgtcgtcagctcgtgtcgtgagatgttgggttaagtcccgcaac |
| | | gagcgcaacccttgtcttctgttaccagcattaagttggggactcaggagagactgccggtgacaaaccggaggaagg |
| | | tggggatgacgtcaaatcatcatgccccttatggcctgggctacacacgtactacaatggcgcctacaaagagcagcg |
| | | acaccgcgaggtggagcgaatctcataaagggcgtctcagttcggattgaagtctgcaactcgacttcatgaagtcgga |
| | | atcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagt |
| | | cgtaacaaggtatccctacgggaacgtggggatggatcacctccttt |
| Eubacterium_sp_3_1_ | SEQ ID | atggagagtttgatcctggctcaggatgaacgctggcggcatgcctaatacatgcaagtcgaacgaagtctttaggaag |
| 31 | NO: 354 | cttgcaccaaagagacttagtggcgaacgggtgagtaacacgtaggtaacctgcccatgtgcccgggataactgctgg |
| | | aaacggtagctaaaaccggataggtatgagggaggcatcttcctcatattaaagcaccttcgggtgtgaacatggatgg |
| | | acctgcggcgcattagctggttggtgaggtaacggcccaccaaggcgatgatgcgtagccgacctgagagggtgaac |
| | | ggccacattgggactgagacacggcccaaactcctacgggaggcagcagtagggaattttcgtcaatggggggaac |
| | | cctgaacgagcaatgccgcgtgtgtgaagaaggtatcggatcgtaaagcactgagtaagtgaagaatgccatataga |
| | | ggaaatgctatgtgggtgacggtagcttaccagaaagccacggctaactacgtgccagcagccgcggtaatacgtag |
| | | gtggcaagcgttatccggaatcattgggcgtaaagggtgcgtaggtggcacgataagtctgaagtaaaaggcaacag |
| | | ctcaactgagtatgctttggaaactgtcgagctagagtgcagaagagggcgatggaattccatgtgtagcggtaaaatg |
| | | cgtagatatatggaggaacaccagtggcgaaggcggtcgcctggtctgtaactgacactgatgcacgaaagcgtggg |
| | | gagcaaataggattagataccctagtagtccacgccgtaaacgatgagaactaagtgttggagagattcagtgctgcag |
| | | ttaacgcaataagttctccgcctggggagtatgcacgcaagtgtgaaactcaaaggaattgacgggggcccgcacaag |
| | | cggtggagtatgtggtttaattcgaagcaacgcgaagaaccttaccaggccttgacatggatataaatgttctagagata |
| | | gaaagatagctatatatcacacaggtggtgcatggttgtcgtcagctcgtgtcgtgagatgttgggttaagtcccgcaac |
| | | gagcgcaacccttgtcttctgttaccagcattaggttggggactcaggagagactgccggtgacaaaccggaggaagg |
| | | tggggatgacgtcaaatcatcatgccccttatggcctgggctacacacgtactacaatggcgcctacaaagagcagcg |
| | | acaccgcgaggtggagcgaatctcataaagggcgtctcagttcggattgaagtctgcaactcgacttcatgaagtcgga |
| | | atcgctagtaatcgcagatcagcatgctgcggtgaatacgttctcgggccttgtacacaccgcccgtcaaaccatggga |
| | | gttggtaatacccgaagccggtggcataaccgcaaggagtgagccgtcgaaggtaggaccgatgactggggttaagt |
| | | cgtaacaaggtatccctacgggaacgtggggatggatcacctccttt |
| Morganella_morganii | SEQ ID | ggggataactactggaaacggtagctaataccgcataatgtcttcggaccaaagcgggggacctccgggcctcgcgc |
| | NO: 355 | catcagatgaacccatatgggattagctagtaggtgaggtaacggcttacctaggcgacgatccctagctggtctgaga |
| | | ggatgatcagccacactgggactgagacacggcccagactcctacgggaggcagcagtggggaatattgcacaatg |
| | | ggcgcaagcctgatgcagccatgccgcgtgtatgaagaaggccttcgggttgtaaagtactttcagtcgggaggaagg |
| | | tgtcaaggttaataaccttggcaattgacgttaccgacagaagaagcaccggctaactccgtgccagcagccgcggta |
| | | atacggagggtgcaagcgttaatcggaattactgggcgtaaagcgcacgcaggcggagattgagtcagatgtgaaat |
| | | ccccgggataacccgggaattgcatctgatactggtcagctagagtcttgtagaggggggtagaattccatgtgtagc |
| | | ggtgaaatgcgtagagatgtggaggaataccggtggcgaaggcggccccctggacaaagactgacgctcaggtgcg |
| | | aaagcgtggggagcaaacaggattagataccctggtagtccacgctgtaaacgatgtcgacttggaggttgtgcccttg |
| | | aggcgtggcttccggagctaacgcgttaagtcgaccgcctggggagtacggccgcaaggttaaaactcaaatgaattg |
| | | acgggggcccgcacaagcggtggagcatgtggtttaattcgatgcaacgcgaagaaccttacctactcttgacatcca |
| | | gagaacttagcagagatgctttggtgccttcgggaactctgagacaggtgctgcatggctgtcgtcagctcgtgttgtga |
| | | aatgttgggttaagtcccgcaacgagcgcaacccttatcctttgttgccagcgcgtgatggcgggaactcaaaggagac |
| | | tgccggtgataaaccggaggaaggtggggatgacgtcaagtcatcatggcccttacgagtagggctacacacgtgcta |
| | | caatggcgtatacaaagggaagcgaccccgcgagggcaagcggaactcataaagtacgtcgtagtccggattggagt |
| | | ctgcaactcgactccatgaagtcggaatcgctagtaatcgtagatcagaatgctacggtgaatacgttcccgggccttgt |
| | | acacaccgcccgtcacaccatgggagtgggttgcaaaagaagtaggtagcttaacctccgggagggcgcttaccactt |
| | | tgtgattcatgactggggtgaagtcgtaacaaggtaaccgtaggggaacctgcggaggatcacctcctt |
| Parabacteroides_ | SEQ ID | cgaagagtttgatcctggctcaggatgaacgctagcgacaggcttaacacatgcaagtcgaggggcagcacaggtag |
| distasanis | NO: 356 | caataccgggtggcgaccggcgcacgggtgagtaacgcgtatgcaacttacctatcagagggggataacccggcga |
| | | aagtcggactaataccgcatgaagcaggggtcccgcatggggatatttgctaaagattcatcgctgatagataggcatg |
| | | cgaccattaggcagaggcggggtaacggcccaccaaaccgacgatggataggggactgagaggaaggtccccca |
| | | cattggtactgagacacggaccaaactcctacgggaggcagcagtgaggaatattggtcaatgggcgtaagcctgaa |
| | | ccagccaagtcgcgtgagggatgaaggttctatggatcgtaaacctcttttataagggaataaagtgcgggacgtgtcct |
| | | gattgtatgtaccttatgaataaggatcggctaactccgtgccagcagccgcggtaatacggaggatccgagcgttatc |
| | | cggatttattgggataaagggtgcgtaggcggcctataagtcagcggtgaaagtctgtggctcaaccatagaattgccg |
| | | ttgaaactggggggcttgagtatgatgaggcaggcggaatgcgtggtgtagcggtgaaatgatagatatcacgcaga |
| | | accccgattgcgaaggcagcctgccaagccatgactgacgctgatgcacgaaagcgtggggatcaaacaggattag |
| | | ataccctggtagtccacgcagtaaacgatgatcactagctgatgcgatacagtgtaagcggcacagcgaaagcgttaa |
| | | gtgatccacctggggagtacgccggcaacggtgaaactcaaaggaattgacgggggcccgcacaagcggaggaac |
| | | atgtggataattcgatgatacgcgaggaaccttacccgggtagaacgcattcggaccgaggtggaaacaccattctag |
| | | caatagccgtttgcgaggtgctgcatggttgtcgtcagctcgtgccgtgaggtgtcggcttaagtgccataacgagcgc |
| | | aacccttgccactagttactaacaggtaaagctgaggactctggtgggactgccagcgtaagctgcgaggaaggcgg |
| | | ggatgacgtcaaatcagcacggccatacatccggggcgacacacgtgttacaatggcgtggacaaagggaggcca |
| | | cctggcgacagggagcgaatccccaaaccacgtctcagttcggatcggagtctgcaacccgactccgtgaagctgga |
| | | ttcgctagtaatcgcgcatcagccatggcgcggtgaatacgttcccgggccttgtacacaccgcccgtcaagccatgg |
| | | gagccgggggtacctgaagtccgtaaccgcgaggatcggcctagggtaaaactggtgactggggctaagtcgtaaca |
| | | aggtagccgtaccggaaggtgcggctggaacacctccat |
| Parabacteroides_ | SEQ ID | cgaagagtttgatcctggctcaggatgaacgctagcgacaggcttaacacatgcaagtcgaggggcagcacaggtag |
| distasanis | NO: 357 | caataccgggtggcgaccggcgcacgggtgagtaacgcgtatgcaacttgcctatcagagggggataacccggcga |
| | | aagtcggactaataccgcatgaagcaggggccccgcatggggatatttgctaaagattcatcgctgatagataggcatg |
| | | cgaccattaggcagaggcggggtaacggcccaccaaaccgacgatggataggggactgagaggaaggtccccca |
| | | cattggtactgagacacggaccaaactcctacgggaggcagcagtgaggaatattggtcaatgggcgcgagcctgaa |
| | | ccagccaagtcgcgtgagggatgaaggttctatggatcgtaaacctcttttataagggaataaagtgcgggacgtgtcct |
| | | gattgtatgtaccttatgaataaggatcggctaactccgtgccagcagccgcggtaatacggaggatccgagcgttatc |
| | | cggatttattgggtttaaagggtgcgtaggcggccttttaagtcagcggtgaaagtctgtggctcaaccatagaattgccg |
| | | ttgaaactggggggcttgagtatgatgaggcaggcggaatgcgtggtgtagcggtgaaatgatagatatcacgcaga |
| | | accccgattgcgaaggcagcctgccaagccatgactgacgctgatgcacgaaagcgtggggatcaaacaggattag |
| | | ataccctggtagtccacgcagtaaacgatgatcactagctgatgcgatacagtgtaagcggcacagcgaaagcgttaa |
| | | gtgatccacctggggagtacgccggcaacggtgaaactcaaaggaattgacgggggcccgcacaagcggaggaac |
| | | atgtggataattcgatgatacgcgaggaaccttacccgggtagaacgcattcggaccgaggtggaaacaccattctag |
| | | caatagccgtttgcgaggtgctgcatggttgtcgtcagctcgtgccgtgaggtgtcggcttaagtgccataacgagcgc |
| | | aacccttgccactagttactaacaggtaaagctgaggactctggtgggactgccagcgtaagctgcgaggaaggcgg |
| | | ggatgacgtcaaatcagcacggccatacatccggggcgacacacgtgttacaatggcgtggacaaagggatgccac |
| | | ctggcgacagggagcgaatccccaaaccacgtctcagttcggatcggagtctgcaacccgactccgtgaagctggatt |
| | | cgctagtaatcgcgcatcagccatggcgcggtgaatacgttcccgggccttgtacacaccgcccgtcaagccatggga |
| | | gccgggggtacctgaagtccgtaaccgaaaggatcggcctagggtaaaactggtgactggggctaagtcgtaacaag |
| | | gtagccgtaccggaaggtgcggctggaacacctccat |
| Parabacteroides_ | SEQ ID | cgaagagtttgatcctggctcaggatgaacgctagcgacaggcttaacacatgcaagtcgaggggcagcacaggtag |
| distasanis | NO: 358 | caataccgggtggcgaccggcgcacgggtgagtaacgcgtatgcaacttacctatcagagggggataacccggcga |
| | | aagtcggactaataccgcatgaagcaggggtcccgcatggggatatttgctaaagattcatcgctgatagataggcatg |
| | | cgttccattaggcagttggcggggtaacggcccaccaaaccgacgatggataggggttctgagaggaaggtccccca |
| | | cattggtactgagacacggaccaaactcctacgggaggcagcagtgaggaatattggtcaatgggcgcgagcctgaa |
| | | ccagccaagtcgcgtgagggatgaaggttctatggatcgtaaacctcttttataagggaataaagtgcgggacgtgtcct |
| | | gttttgtatgtaccttatgaataaggatcggctaactccgtgccagcagccgcggtaatacggaggatccgagcgttatc |
| | | cggatttattgggtttaaagggtgcgtaggcggccttttaagtcagcggtgaaagtctgtggctcaaccatagaattgccg |
| | | ttgaaactggggggcttgagtatgtttgaggcaggcggaatgcgtggtgtagcggtgaaatgatagatatcacgcaga |
| | | accccgattgcgaaggcagcctgccaagccatgactgacgctgatgcacgaaagcgtggggatcaaacaggattag |
| | | ataccctggtagtccacgcagtaaacgatgatcactagctgtttgcgatacagtgtaagcggcacagcgaaagcgttaa |
| | | gtgatccacctggggagtacgccggcaacggtgaaactcaaaggaattgacgggggcccgcacaagcggaggaac |
| | | atgtggtttaattcgatgatacgcgaggaaccttacccgggtttgaacgcattcggaccgaggtggaaacaccttttctag |
| | | caatagccgtttgcgaggtgctgcatggttgtcgtcagctcgtgccgtgaggtgtcggcttaagtgccataacgagcgc |
| | | aaccatgccactagttactaacaggtgatgctgaggactctggtgggactgccagcgtaagctgcgaggaaggcgg |
| | | ggatgacgtcaaatcagcacggcccttacatccggggcgacacacgtgttacaatggcgtggacaaagggaagcca |
| | | cctggcgacagggagcgaatccccaaaccacgtctcagttcggatcggagtctgcaacccgactccgtgaagctgga |
| | | ttcgctagtaatcgcgcatcagccatggcgcggtgaatacgttcccgggccttgtacacaccgcccgtcaagccatgg |
| | | gagccgggggtacctgaagtccgtaaccgaaaggatcggcctagggtaaaactggtgactggggctaagtcgtaaca |
| | | aggtagccgtaccggaaggtgcggctggaacacctccttt |
| Parabacteroides_ | SEQ ID | cgaagagtttgatcctggctcaggatgaacgctagcgacaggcttaacacatgcaagtcgaggggcagcgggggtag |
| distasanis | NO: 359 | caatacccgccggcgaccggcgcacgggtgagtaacgcgtatgcaacttacctatcagagggggataacccggcga |
| | | aagtcggactaataccgcatgaagcaggggccccgcatggggatatttgctaaagattcatcgctgatagataggcatg |
| | | cgaccattaggcagaggcggggtaacggcccaccaaaccgacgatggataggggactgagaggaaggtccccca |
| | | cattggtactgagacacggaccaaactcctacgggaggcagcagtgaggaatattggtcaatgggcgcgagcctgaa |
| | | ccagccaagtcgcgtgagggatgaaggttctatggatcgtaaacctcttttataagggaataaagtgcgggacgtgtcct |
| | | gattgtatgtaccttatgaataaggatcggctaactccgtgccagcagccgcggtaatacggaggatccgagcgttatc |
| | | cggatttattgggataaagggtgcgtaggcggcctataagtcagcggtgaaagtctgtggctcaaccatagaattgccg |
| | | ttgaaactggggggcttgagtatgtttgaggcaggcggaatgcgtggtgtagcggtgaaatgcttagatatcacgcaga |
| | | accccgattgcgaaggcagcctgccaagccatgactgacgctgatgcacgaaagcgtggggatcaaacaggattag |
| | | ataccctggtagtccacgcagtaaacgatgatcactagctgtttgcgatacagtgtaagcggcacagcgaaagcgttaa |
| | | gtgatccacctggggagtacgccggcaacggtgaaactcaaaggaattgacgggggcccgcacaagcggaggaac |
| | | atgtggtttaattcgatgatacgcgaggaaccttacccgggtttgaacgcattcggaccgaggtggaaacaccttttctag |
| | | caatagccgtttgcgaggtgctgcatggttgtcgtcagctcgtgccgtgaggtgtcggcttaagtgccataacgagcgc |
| | | aacccttgccactagttactaacaggtaatgctgaggactctggtgggactgccagcgtaagctgcgaggaaggcggg |
| | | gatgacgtcaaatcagcacggcccttacatccggggcgacacacgtgttacaatggcgtggacaaagggaggccac |
| | | ctggcgacagggagcgaatccccaaaccacgtctcagttcggatcggagtctgcaacccgactccgtgaagctggatt |
| | | cgctagtaatcgcgcatcagccatggcgcggtgaatacgttcccgggccttgtacacaccgcccgtcaagccatggga |
| | | gccgggggtacctgaagtccgtaaccgcgaggatcggcctagggtaaaactggtgactggggctaagtcgtaacaag |
| | | gtagccgtaccggaaggtgcggctggaacacctccat |
| Parabacteroides_ | SEQ ID | cgaagagtttgatcctggctcaggatgaacgctagcgacaggcttaacacatgcaagtcgaggggcagcacaggtag |
| distasanis | NO: 360 | caataccgggtggcgaccggcgcacgggtgagtaacgcgtatgcaacttacctatcagagggggataacccggcga |
| | | aagtcggactaataccgcatgaagcaggggtcccgcatggggatatttgctaaagattcatcgctgatagataggcatg |
| | | cgaccattaggcagaggcggggtaacggcccaccaaaccgacgatggataggggactgagaggaaggtccccca |
| | | cattggtactgagacacggaccaaactcctacgggaggcagcagtgaggaatattggtcaatgggcgtaagcctgaa |
| | | ccagccaagtcgcgtgagggatgaaggactatggatcgtaaacctctatataagggaataaagtgcgggacgtgtcct |
| | | gattgtatgtaccttatgaataaggatcggctaactccgtgccagcagccgcggtaatacggaggatccgagcgttatc |
| | | cggatttattgggataaagggtgcgtaggcggcctataagtcagcggtgaaagtctgtggctcaaccatagaattgccg |
| | | ttgaaactggggggcttgagtatgatgaggcaggcggaatgcgtggtgtagcggtgaaatgatagatatcacgcaga |
| | | accccgattgcgaaggcagcctgccaagccatgactgacgctgatgcacgaaagcgtggggatcaaacaggattag |
| | | ataccctggtagtccacgcagtaaacgatgatcactagctgatgcgatacagtgtaagcggcacagcgaaagcgttaa |
| | | gtgatccacctggggagtacgccggcaacggtgaaactcaaaggaattgacgggggcccgcacaagcggaggaac |
| | | atgtggataattcgatgatacgcgaggaaccttacccgggtagaacgcattcggaccgaggtggaaacaccattctag |
| | | caatagccgtttgcgaggtgctgcatggttgtcgtcagctcgtgccgtgaggtgtcggcttaagtgccataacgagcgc |
| | | aaccatgccactagttactaacaggtaaagctgaggactctggtgggactgccagcgtaagctgcgaggaaggcgg |
| | | ggatgacgtcaaatcagcacggcccttacatccggggcgacacacgtgttacaatggcgtggacaaagggaagcca |
| | | cctggcgacagggagcgaatccccaaaccacgtctcagttcggatcggagtctgcaacccgactccgtgaagctgga |
| | | ttcgctagtaatcgcgcatcagccatggcgcggtgaatacgttcccgggccttgtacacaccgcccgtcaagccatgg |
| | | gagccgggggtacctgaagtccgtaaccgcgaggatcggcctagggtaaaactggtgactggggctaagtcgtaaca |
| | | aggtagccgtaccggaaggtgcggctggaacacctccat |
| Citrobacter_freundii | SEQ ID | attggagagatgatcctggctcaggacgaacgctggcggcgtgcctaatacatgcaagtcgagcgagtcccacggg |
| | NO: 361 | ggctagcggcggacgggtgagtaacacgtaggcaacctgcctctcagactgggataacatagggaaacttatgctaat |
| | | accggataggtttaggatcgcatgatctgaaaagaaaagatggcttttcgctatcactgggagatgggcctgcggcgca |
| | | ttagctagttggtggggtaacggcctaccaaggcgacgatgcgtagccgacctgagagggtgaccggccacactgg |
| | | gactgagacacggcccagactcctacgggaggcagcagtagggaattttccacaatggacgaaagtctgatggagca |
| | | acgccgcgtgaacgatgaaggtcttcggattgtaaagactgttgtcagggacgaacacgtaccgttcgaatagggcgg |
| | | taccttgacggtacctgacgagaaagccacggctaactacgtgccagcagccgcggtaatacgtaggtggcaagcgtt |
| | | gtccggatttattgggcgtaaagcgcgcgcaggcggctatgtaagtctggtgttaaagcccggggctcaaccccggttc |
| | | gcatcggaaactgtgtagcttgagtgcagaagaggaaagcggtattccacgtgtagcggtgaaatgcgtagagatgtg |
| | | gaggaacaccagtggcgaaggcggctttctggtctgtaactgacgctgaggcgcgaaagcgtggggagcaaacagg |
| | | attagataccctggtagtccacgccgtaaacgatgagtgctaggtgttgggggtttcaataccctcagtgccgcagctaa |
| | | cgcaataagcactccgcctggggagtacgctcgcaagagtgaaactcaaaggaattgacgggggcccgcacaagcg |
| | | gtggagcatgtggtttaattcgaagcaacgcgaagaaccttaccaggtcttgacatcccgctgaccgctctggagacag |
| | | agcacccttcggggcagcggtgacaggtggtgcatggttgtcgtcagctcgtgtcgtgagatgttgggttaagtcccgc |
| | | aacgagcgcaacccttatctttagttgccagcattcagttgggcactctagagagactgccgtcgacaagacggaggaa |
| | | ggcggggatgacgtcaaatcatcatgccccttatgacctgggctacacacgtgctacaatggaggtacaacgggacg |
| | | ctacctcgcgagaggacgccaatctcttaaaaccaatctcagttcggattgtaggctgcaactcgcctacatgaagtcgg |
| | | aatcgctagtaatcgcggatcagcatgccgcggtgaatacgttcccgggccttgtacacaccgcccgtcacaccacgg |
| | | gagtttgcaacacccgaagtcggtgaggtaaccgcaaggagccagccgccgaaggtggggtagatgactggggtga |
| | | agtcgtaacaaggtatccgtaccggaaggtgcggatggatcacctccat |
| Citrobacter_freundii | SEQ ID | ttgaagagtttgatcatggctcagattgaacgctggcggcaggcctaacacatgcaagtcgaacggtagcacagagga |
| | NO: 362 | gcttgctcatgggtgacgagtggcggacgggtgagtaatgtctgggaaactgcccgatggagggggataactactgg |
| | | aaacggtagctaataccgcataacgtcgcaagaccaaagagggggaccttcgggcctcttgccatcggatgtgccca |
| | | gatgggattagctagtaggtggggtaacggctcacctaggcgacgatccctagctggtctgagaggatgaccagcca |
| | | cactggaactgagacacggtccagactcctacgggaggcagcagtggggaatattgcacaatgggcgcaagcctgat |
| | | gcagccatgccgcgtgtatgaagaaggccttcgggttgtaaagtactttcagcgaggaggaaggcgttgtggttaataa |
| | | ccgcagcgattgacgttactcgcagaagaagcaccggctaactccgtgccagcagccgcggtaatacggagggtgc |
| | | aagcgttaatcggaattactgggcgtaaagcgcacgcaggcggtctgtcaagtcggatgtgaaatccccgggctcaac |
| | | ctgggaactgcatccgaaactggcaggctagagtcttgtagaggggggtagaattccaggtgtagcggtgaaatgcgt |
| | | agagatctggaggaataccggtggcgaaggcggccccctggacaaagactgacgctcaggtgcgaaagcgtgggg |
| | | agcaaacaggattagataccctggtagtccacgccgtaaacgatgtcgacttggaggttgtgcccttgaggcgtggctt |
| | | ccggagctaacgcgttaagtcgaccgcctggggagtacggccgcaaggttaaaactcaaatgaattgacgggggccc |
| | | gcacaagcggtggagcatgtggtttaattcgatgcaacgcgaagaaccttacctactatgacatccagagaacttagca |
| | | gagatgctttggtgccttcgggaactctgagacaggtgctgcatggctgtcgtcagctcgtgttgtgaaatgttgggttaa |
| | | gtcccgcaacgagcgcaacccttatcctttgagccagcggacggccgggaactcaaaggagactgccagtgataaa |
| | | ctggaggaaggtggggatgacgtcaagtcatcatggcccttacgagtagggctacacacgtgctacaatggcatatac |
| | | aaagagaagcgacctcgcgagagcaagcggacctcataaagtatgtcgtagtccggattggagtctgcaactcgactc |
| | | catgaagtcggaatcgctagtaatcgtggatcagaatgccacggtgaatacgacccgggccttgtacacaccgcccgt |
| | | cacaccatgggagtgggttgcaaaagaagtaggtagataaccttcgggagggcgcttaccactttgtgattcatgactg |
| | | gggtgaagtcgtaacaaggtaaccgtaggggaacctgcggttggatcacctcctt |
| Lactococcus_lactis | SEQ ID | atttgagagtttgatcctggctcaggacgaacgctggcggcgtgcctaatacatgcaagttgagcgctgaaggttggta |
| | NO: 363 | cttgtaccgactggatgagcagcgaacgggtgagtaacgcgtggggaatctgcctttgagcgggggacaacatttgga |
| | | aacgaatgctaataccgcataaaaactttaaacacaagttttaagtttgaaagatgcaattgcatcactcaaagatgatccc |
| | | gcgttgtattagctagttggtgaggtaaaggctcaccaaggcgatgatacatagccgacctgagagggtgatcggcca |
| | | cattgggactgagacacggcccaaactcctacgggaggcagcagtagggaatcttcggcaatggacgaaagtctgac |
| | | cgagcaacgccgcgtgagtgaagaaggttttcggatcgtaaaactctgaggtagagaagaacgaggtgagagtgga |
| | | aagctcatcaagtgacggtaactacccagaaagggacggctaactacgtgccagcagccgcggtaatacgtaggtcc |
| | | cgagcgttgtccggatttattgggcgtaaagcgagcgcaggtggtttattaagtctggtgtaaaaggcagtggctcaacc |
| | | attgtatgcattggaaactggtagacttgagtgcaggagaggagagtggaattccatgtgtagcggtgaaatgcgtagat |
| | | atatggaggaacaccggtggcgaaagcggctctctggcctgtaactgacactgaggctcgaaagcgtggggagcaa |
| | | acaggattagataccctggtagtccacgccgtaaacgatgagtgctagatgtagggagctataagttctctgtatcgcag |
| | | ctaacgcaataagcactccgcctggggagtacgaccgcaaggttgaaactcaaaggaattgacgggggcccgcaca |
| | | agcggtggagcatgtggtttaattcgaagcaacgcgaagaaccttaccaggtcttgacatactcgtgctattcctagaga |
| | | taggaagttccttcgggacacgggatacaggtggtgcatggttgtcgtcagctcgtgtcgtgagatgttgggttaagtcc |
| | | cgcaacgagcgcaacccctattgttagttgccatcattaagttgggcactctaacgagactgccggtgataaaccggag |
| | | gaaggtggggatgacgtcaaatcatcatgccccttatgacctgggctacacacgtgctacaatggatggtacaacgagt |
| | | cgcgagacagtgatgtttagctaatctataaaaccattctcagttcggattgtaggctgcaactcgcctacatgaagtcg |
| | | gaatcgctagtaatcgcggatcagcacgccgcggtgaatacgttcccgggccttgtacacaccgcccgtcacaccacg |
| | | ggagttgggagtacccgaagtaggttgcctaaccgcaaggagggcgcttcctaaggtaagaccgatgactggggtga |
| | | agtcgtaacaaggtagccgtatcggaaggtgcggctggatcacctcctt |
| Lactococcus_lactis | SEQ ID | atttgagagtttgatcctggctcaggacgaacgctggcggcgtgcctaatacatgcaagttgagcgctgaaggttggta |
| | NO: 364 | cttgtaccgactggatgagcagcgaacgggtgagtaacgcgtggggaatctgcctttgagcgggggacaacatttgga |
| | | aacgaatgctaataccgcataaaaactttaaacacaagttttaagtttgaaagatgcaattgcatcactcaaagatgatccc |
| | | gcgttgtattagctagttggtgaggtaaaggctcaccaaggcgatgatacatagccgacctgagagggtgatcggcca |
| | | cattgggactgagacacggcccaaactcctacgggaggcagcagtagggaatcttcggcaatggacgaaagtctgac |
| | | cgagcaacgccgcgtgagtgaagaaggtatcggatcgtaaaactctgaggtagagaagaacgdggtgagagtgga |
| | | aagctcatcaagtgacggtaactacccagaaagggacggctaactacgtgccagcagccgcggtaatacgtaggtcc |
| | | cgagcgttgtccggatttattgggcgtaaagcgagcgcaggtggtttattaagtctggtgtaaaaggcagtggctcaacc |
| | | attgtatgcattggaaactggtagacttgagtgcaggagaggagagtggaattccatgtgtagcggtgaaatgcgtagat |
| | | atatggaggaacaccggtggcgaaagcggctctctggcctgtaactgacactgaggctcgaaagcgtggggagcaa |
| | | acaggattagataccctggtagtccacgccgtaaacgatgagtgctagatgtagggagctataagttctctgtatcgcag |
| | | ctaacgcaataagcactccgcctggggagtacgaccgcaaggttgaaactcaaaggaattgacgggggcccgcaca |
| | | agcggtggagcatgtggtttaattcgaagcaacgcgaagaaccttaccaggtcttgacatactcgtgctattcctagaga |
| | | taggaagttccttcgggacacgggatacaggtggtgcatggttgtcgtcagctcgtgtcgtgagatgttgggttaagtcc |
| | | cgcaacgagcgcaacccctattgdagagccatcattaagttgggcactctaacgagactgccggtgataaaccggag |
| | | gaaggtggggatgacgtcaaatcatcatgccccttatgacctgggctacacacgtgctacaatggatggtacaacgagt |
| | | cgcgagacagtgatgtttagctaatctcttaaaaccattctcagttcggattgtaggctgcaactcgcctacatgaagtcg |
| | | gaatcgctagtaatcgcggatcagcacgccgcggtgaatacgttcccgggccttgtacacaccgcccgtcacaccacg |
| | | ggagttgggagtacccgaagtaggttgcctaaccgcaaggagggcgcttcctaaggtaagaccgatgactggggtga |
| | | agtcgtaacaaggtagccgtatcggaaggtgcggctggatcacctcctt |
| Alistipes_onderdonkii | SEQ ID | atggagagtttgatcctggctcaggatgaacgctagcggcaggcctaacacatgcaagtcgaggggcatcgggattg |
| | NO: 365 | aagcttgcttcaattgccggcgaccggcgcacgggtgcgtaacgcgtatgtaacctacctataacaggggcataacact |
| | | gagaaattggtactaattccccataatattcggagaggcatctctccgggttgaaaactccggtggttatagatggacatg |
| | | cgttgtattagctagttggtgaggtaacggctcaccaaggcaacgatacatagggggactgagaggttaaccccccac |
| | | actggtactgagacacggaccagactcctacgggaggcagcagtgaggaatattggtcaatggacgcaagtctgaac |
| | | cagccatgccgcgtgcaggaagacggctctatgagagtaaactgcttagtacgagggtaaactcacctacgtgtaggt |
| | | gactgaaagtatcgtacgaataaggatcggctaactccgtgccagcagccgcggtaatacggaggattcaagcgttat |
| | | ccggatttattgggtttaaagggtgcgtaggcggtttgataagttagaggtgaaatcccggggcttaactccggaactgc |
| | | ctctaatactgttagactagagagtagagcggtaggcggaatgtatggtgtagcggtgaaatgcttagagatcatacag |
| | | aacaccgattgcgaaggcagcttaccaaactatatctgacgttgaggcacgaaagcgtggggagcaaacaggattag |
| | | ataccctggtagtccacgcagtaaacgatgataactcgdgtcggcgatacacagtcggtgactaagcgaaagcgataa |
| | | gttatccacctggggagtacgttcgcaagaatgaaactcaaaggaattgacgggggcccgcacaagcggaggaacat |
| | | gtggtttaattcgatgatacgcgaggaaccttacccgggcttgaaagttactgacgattctggaaacaggatttcccttcg |
| | | gggcaggaaactaggtgctgcatggagtcgtcagctcgtgccgtgaggtgtcgggttaagtcccataacgagcgcaa |
| | | cccctaccgttagttgccatcaggtcaagctgggcactctggcgggactgccggtgtaagccgagaggaaggtgggg |
| | | atgacgtcaaatcagcacggcccttacgtccggggctacacacgtgttacaatggtaggtacagagggtcgctacccc |
| | | gcgaggggatgccaatctcgaaagcctatctcagttcggattggaggctgaaacccgcctccatgaagttggattcgct |
| | | agtaatcgcgcatcagccatggcgcggtgaatacgttcccgggccttgtacacaccgcccgtcaagccatggaagctg |
| | | ggggtgcctgaagttcgtgaccgcaaggagcgacctagggcaaaaccggtaactggggctaagtcgtaacaaggta |
| | | gccgtaccggaaggtgcggctggaacacctccttt |
| Alistipes_onderdonkii | SEQ ID | atggagagtttgatcctggctcaggatgaacgctagcggcaggcctaacacatgcaagtcgaggggcatcgggattg |
| | NO: 366 | aagcttgatcaattgccggcgaccggcgcacgggtgcgtaacgcgtatgtaacctacctataacaggggcataacact |
| | | gagaaattggtactaattccccataatattcggagaggcatctctccgggttgaaaactccggtggttatagatggacatg |
| | | cgttgtattagctagttggtgaggtaacggctcaccaaggcaacgatacatagggggactgagaggttaaccccccac |
| | | actggtactgagacacggaccagactcctacgggaggcagcagtgaggaatattggtcaatggacgcaagtctgaac |
| | | cagccatgccgcgtgcaggaagacggctctatgagttgtaaactgcattgtacgagggtaaactcacctacgtgtaggt |
| | | gactgaaagtatcgtacgaataaggatcggctaactccgtgccagcagccgcggtaatacggaggattcaagcgttat |
| | | ccggatttattgggtttaaagggtgcgtaggcggtttgataagttagaggtgaaatcccggggcttaactccggaactgc |
| | | ctctaatactgttagactagagagtagttgcggtaggcggaatgtatggtgtagcggtgaaatgcttagagatcatacag |
| | | aacaccgattgcgaaggcagcttaccaaactatatctgacgttgaggcacgaaagcgtggggagcaaacaggattag |
| | | ataccctggtagtccacgcagtaaacgatgataactcgdgtcggcgatacacagtcggtgactaagcgaaagcgataa |
| | | gttatccacctggggagtacgttcgcaagaatgaaactcaaaggaattgacgggggcccgcacaagcggaggaacat |
| | | gtggtttaattcgatgatacgcgaggaaccttacccgggcttgaaagttactgacgattctggaaacaggatttcccttcg |
| | | gggcaggaaactaggtgctgcatggttgtcgtcagctcgtgccgtgaggtgtcgggttaagtcccataacgagcgcaa |
| | | cccctaccgttagttgccatcaggtcaagctgggcactctggcgggactgccggtgtaagccgagaggaaggtgggg |
| | | atgacgtcaaatcagcacggcccttacgtccggggctacacacgtgttacaatggtaggtacagagggtcgctacccc |
| | | gcgaggggatgccaatctcgaaagcctatctcagttcggattggaggctgaaacccgcctccatgaagttggattcgct |
| | | agtaatcgcgcatcagccatggcgcggtgaatacgttcccgggccttgtacacaccgcccgtcaagccatggaagctg |
| | | ggggtgcctgaagttcgtgaccgcaaggagcgacctagggcaaaaccggtaactggggctaagtcgtaacaaggta |
| | | gccgtaccggaaggtgcggctggaacacctccttt |
| Campylobacter_ | SEQ ID | atggagagtttgatcctggctcagagtgaacgctggcggcgtgcctaatacatgcaagtcgaacggacaagtaagagc |
| curvus | NO: 367 | ttgctcttatgagttagtggcgcacgggtgagtaatgtatagctaatctgccccatagtggaggacaacagttggaaacg |
| | | actgctaatactccatactccatttatatataagtataaatgggaaagtcgaaccctatatattgtttatttatcaaatacctac |
| | | ggcgcaaccgaaagttgcggtgtgattaggtgggtcaagggagcgtagctccttgtcgcaaagacggactttgttcgt |
| | | ctgcgaagtaaataatatatagggtttctcgctatgggatgaggctatatcgtatcagctagttggtaaggtaatggcttac |
| | | caaggctatgacgcgtaactggtctgagaggatgatcagtcacactggaactgagacacggtccagactcctacggga |
| | | ggcagcagtagggaatattgctcaatgggggaaaccctgaagcagcaacgccgcgtggaggatgacactdtcggag |
| | | cgtaaactccdttatggggaagaaatttgacggtacccaaggaataagcaccggctaactccgtgccagcagccgcg |
| | | gtaatacggagggtgcaagcgttactcggaatcactgggcgtaaaggacgcgtaggcggattatcaagtctcttgtgaa |
| | | atccaatggcttaaccattgaactgcttgagaaactgataatctagagtgagggagaggtagatggaattggtggtgtag |
| | | gggtaaaatccgtagagatcaccaggaatacccattgcgaaggcgatctgctggaactcaactgacgctaatgcgtga |
| | | aagcgtggggagcaaacaggattagataccctggtagtccacgccctaaacgatgtatactagttgttgctgagctagtc |
| | | ttggcagtaatgcacctaacggattaagtataccgcctggggagtacggtcgcaagattaaaactcaaaggaatagacg |
| | | gggacccgcacaagcggtggagcatgtggtttaattcgaagatacgcgaagaaccttacccggacttgatatctaacaa |
| | | atcatctagagatagaagagtgtctgcttgcagaaatgttaagacaggtgctgcacggctgtcgtcagctcgtgtcgtga |
| | | gatgttgggttaagtcccgcaacgagcgcaacccacgtcattagttgctaacggttcggccgagcactctaatgagact |
| | | gcatcgcaaggaggaggaaggtgtggacgacgtcaagtcatcatggccatatgtccggggcgacacacgtgctac |
| | | aatggcgtatacaatgagacgcaatatcgcgagatggagcaaatctataaaatacgtcccagttcggattggagtctgc |
| | | aactcgactccatgaagccggaatcgctagtaatcgtagatcagccatgctacggtgaatacgttcccgggtcagtact |
| | | caccgcccgtcacaccatgggagttgatttcactcgaagcccaaataccaaaccggttatggtccacagtggaatcagc |
| | | gactggggtgaagtcgtaacaaggtaaccgtaggagaacctgcggdggatcacctcctt |
| Lactobacillus_ | SEQ ID | tatgagagtttgatcctggctcaggatgaacgccggcggtgtgcctaatacatgcaagtcgaacgcgttggcccgattg |
| fermentum | NO: 368 | attgatggtgcttgcacctgattgattttggtcgccaacgagtggcggacgggtgagtaacacgtaggtaacctgccca |
| | | gaagcgggggacaacatttggaaacagatgctaataccgcataacaacgttgttcgcatgaacaacgcttaaaagatg |
| | | gatctcgctatcacttctggatggacctgcggtgcattagcttgaggtggggtaacggcctaccaaggcgatgatgcat |
| | | agccgagttgagagactgatcggccacaatgggactgagacacggcccatactcctacgggaggcagcagtaggga |
| | | atcttccacaatgggcgcaagcctgatggagcaacaccgcgtgagtgaagaagggtttcggctcgtaaagctctgttgt |
| | | taaagaagaacacgtatgagagtaactgttcatacgttgacggtatttaaccagaaagtcacggctaactacgtgccagc |
| | | agccgcggtaatacgtaggtggcaagcgttatccggatttattgggcgtaaagagagtgcaggcggttttctaagtctga |
| | | tgtgaaagccttcggcttaaccggagaagtgcatcggaaactggataacttgagtgcagaagagggtagtggaactcc |
| | | atgtgtagcggtggaatgcgtagatatatggaagaacaccagtggcgaaggcggctacctggtctgcaactgacgctg |
| | | agactcgaaagcatgggtagcgaacaggattagataccctggtagtccatgccgtaaacgatgagtgctaggtgagg |
| | | agggtttccgcccttcagtgccggagctaacgcattaagcactccgcctggggagtacgaccgcaaggttgaaactca |
| | | aaggaattgacgggggcccgcacaagcggtggagcatgtggtttaattcgaagctacgcgaagaaccttaccaggtct |
| | | tgacatcttgcgccaaccctagagatagggcgtttccttcgggaacgcaatgacaggtggtgcatggtcgtcgtcagct |
| | | cgtgtcgtgagatgagggttaagtcccgcaacgagcgcaacccagttactagttgccagcattaagttgggcactctag |
| | | tgagactgccggtgacaaaccggaggaaggtggggacgacgtcagatcatcatgccccttatgacctgggctacaca |
| | | cgtgctacaatggacggtacaacgagtcgcgaactcgcgagggcaagcaaatctcttaaaaccgttctcagttcggact |
| | | gcaggctgcaactcgcctgcacgaagtcggaatcgctagtaatcgcggatcagcatgccgcggtgaatacgttcccg |
| | | ggccagtacacaccgcccgtcacaccatgagagtagtaacacccaaagtcggtggggtaaccattaggagccagcc |
| | | gcctaaggtgggacagatgattagggtgaagtcgtaacaaggtagccgtaggagaacctgcggctggatcacctcctt |
| | | t |
| Lactobacillus_ | SEQ ID | tatgagagtttgatcctggctcaggatgaacgccggcggtgtgcctaatacatgcaagtcgaacgcgttggcccaattg |
| fermentum | NO: 369 | attgacggtgcttgcacctgattgattaggtcgccaacgagtggcggacgggtgagtaacacgtaggtaacctgccca |
| | | gaagcgggggacaacatttggaaacagatgctaataccgcataacaacgttgttcgcatgaacaacgcttaaaagatg |
| | | gatctcgctatcacttctggatggacctgcggtgcattagcttgaggtggggtaacggcctaccaaggcgatgatgcat |
| | | agccgagttgagagactgatcggccacaatgggactgagacacggcccatactcctacgggaggcagcagtaggga |
| | | atcttccacaatgggcgcaagcctgatggagcaacaccgcgtgagtgaagaagggtttcggctcgtaaagctctgttgt |
| | | taaagaagaacacgtatgagagtaactgacatacgttgacggtatttaaccagaaagtcacggctaactacgtgccagc |
| | | agccgcggtaatacgtaggtggcaagcgttatccggatttattgggcgtaaagagagtgcaggcggattctaagtctga |
| | | tgtgaaagccacggcttaaccggagaagtgcatcggaaactggataacttgagtgcagaagagggtagtggaactcc |
| | | atgtgtagcggtggaatgcgtagatatatggaagaacaccagtggcgaaggcggctacctggtctgcaactgacgctg |
| | | agactcgaaagcatgggtagcgaacaggattagataccctggtagtccatgccgtaaacgatgagtgctaggtgagg |
| | | agggtttccgcccttcagtgccggagctaacgcattaagcactccgcctggggagtacgaccgcaaggttgaaactca |
| | | aaggaattgacgggggcccgcacaagcggtggagcatgtggtttaattcgaagctacgcgaagaaccttaccaggtct |
| | | tgacatcttgcgccaaccctagagatagggcgtaccacgggaacgcaatgacaggtggtgcatggtcgtcgtcagct |
| | | cgtgtcgtgagatgagggttaagtcccgcaacgagcgcaacccagttactagttgccagcattaagttgggcactctag |
| | | tgagactgccggtgacaaaccggaggaaggtggggacgacgtcagatcatcatgccccttatgacctgggctacaca |
| | | cgtgctacaatggacggtacaacgagtcgcgaactcgcgagggcaagcaaatctataaaaccgttctcagttcggact |
| | | gcaggctgcaactcgcctgcacgaagtcggaatcgctagtaatcgcggatcagcatgccgcggtgaatacgttcccg |
| | | ggccagtacacaccgcccgtcacaccatgagagtagtaacacccaaagtcggtggggtaaccattaggagccagcc |
| | | gcctaaggtgggacagatgattagggtgaagtcgtaacaaggtagccgtaggagaacctgcggctggatcacctcctt |
| | | t |
| Lactobacillus_ | SEQ ID | tatgagagtttgatcctggctcaggatgaacgccggcggtgtgcctaatacatgcaagtcgaacgcgttggcccaattg |
| fermentum | NO: 370 | attgatggtgcttgcacctgattgattttggtcgccaacgagtggcggacgggtgagtaacacgtaggtaacctgccca |
| | | gaagcgggggacaacatttggaaacagatgctaataccgcataacaacgttgttcgcatgaacaacgcttaaaagatg |
| | | gatctcgctatcacttctggatggacctgcggtgcattagcttgaggtggggtaacggcctaccaaggcgatgatgcat |
| | | agccgagttgagagactgatcggccacaatgggactgagacacggcccatactcctacgggaggcagcagtaggga |
| | | atcttccacaatgggcgcaagcctgatggagcaacaccgcgtgagtgaagaagggtttcggctcgtaaagctctgttgt |
| | | taaagaagaacacgtatgagagtaactgttcatacgttgacggtatttaaccagaaagtcacggctaactacgtgccagc |
| | | agccgcggtaatacgtaggtggcaagcgttatccggatttattgggcgtaaagagagtgcaggcggattctaagtctga |
| | | tgtgaaagccttcggcttaaccggagaagtgcatcggaaactggataacttgagtgcagaagagggtagtggaactcc |
| | | atgtgtagcggtggaatgcgtagatatatggaagaacaccagtggcgaaggcggctacctggtctgcaactgacgctg |
| | | agactcgaaagcatgggtagcgaacaggattagataccctggtagtccatgccgtaaacgatgagtgctaggtgttgg |
| | | agggtttccgcccttcagtgccggagctaacgcattaagcactccgcctggggagtacgaccgcaaggttgaaactca |
| | | aaggaattgacgggggcccgcacaagcggtggagcatgtggtttaattcgaagctacgcgaagaaccttaccaggtct |
| | | tgacatcttgcgccaaccctagagatagggcgtttccttcgggaacgcaatgacaggtggtgcatggtcgtcgtcagct |
| | | cgtgtcgtgagatgagggttaagtcccgcaacgagcgcaacccagttactagttgccagcattaagagggcactctag |
| | | tgagactgccggtgacaaaccggaggaaggtggggacgacgtcagatcatcatgccccttatgacctgggctacaca |
| | | cgtgctacaatggacggtacaacgagtcgcgaactcgcgagggcaagcaaatctataaaaccgttctcagttcggact |
| | | gcaggctgcaactcgcctgcacgaagtcggaatcgctagtaatcgcggatcagcatgccgcggtgaatacgttcccg |
| | | ggccttgtacacaccgcccgtcacaccatgagagtttgtaacacccaaagtcggtggggtaaccttttaggagccagcc |
| | | gcctaaggtgggacagatgattagggtgaagtcgtaacaaggtagccgtaggagaacctgcggctggatcacctcctt |
| | | t |
| Lactobacillus_ | SEQ ID | tatgagagtttgatcctggctcaggatgaacgccggcggtgtgcctaatacatgcaagtcgaacgcgttggtccaattga |
| fermentum | NO: 371 | ttgacggtgatgcacctgattgattaggtcgccaacgagtggcggacgggtgagtaacacgtaggtaacctgcccag |
| | | aagcgggggacaacatttggaaacagatgctaataccgcataacaacgttgttcgcatgaacaacgtttaaaagatggc |
| | | actcgctatcacttctggatggacctgcggtgcattagcttgaggtggggtaatggcctaccaaggcgatgatgcatag |
| | | ccgagttgagagactgatcggccacaatgggactgagacacggcccatactcctacgggaggcagcagtagggaat |
| | | cttccacaatgggcgcaagcctgatggagcaacaccgcgtgagtgaagaagggtacggctcgtaaagctctgagtta |
| | | aagaagaacacgtatgagagtaactgttcatacgttgacggtatttaaccagaaagtcacggctaactacgtgccagca |
| | | gccgcggtaatacgtaggtggcaagcgttatccggatttattgggcgtaaagagagtgcaggcggattctaagtctgat |
| | | gtgaaagccttcggcttaaccggagaagtgcatcggaaactggataacttgagtgcagaagagggtagtggaactcca |
| | | tgtgtagcggtggaatgcgtagatatatggaagaacaccagtggcgaaggcggctacctggtctgcaactgacgctga |
| | | gactcgaaagcatgggtagcgaacaggattagataccctggtagtccatgccgtaaacgatgagtgctaggtgttgga |
| | | gggtttccgcccttcagtgccggagctaacgcattaagcactccgcctggggagtacgaccgcaaggttgaaactcaa |
| | | aggaattgacgggggcccgcacaagcggtggagcatgtggtttaattcgaagctacgcgaagaaccttaccaggtctt |
| | | gacatcttgcgccaaccctagagatagggcgatccttcgggaacgcaatgacaggtggtgcatggtcgtcgtcagctc |
| | | gtgtcgtgagatgagggttaagtcccgcaacgagcgcaacccagttactagagccagcattaagagggcactctagt |
| | | gagactgccggtgacaaaccggaggaaggtggggacgacgtcagatcatcatgccccttatgacctgggctacacac |
| | | gtgctacaatggacggtacaacgagtcgcaaactcgcgagggcaagcaaatctataaaaccgttctcagttcggactg |
| | | caggctgcaactcgcctgcacgaagtcggaatcgctagtaatcgcggatcagcatgccgcggtgaatacgttcccgg |
| | | gccttgtacacaccgcccgtcacaccatgagagtttgtaacacccaaagtcggtggggtaaccttttaggagccagccg |
| | | cctaaggtgggacagatgattagggtgaagtcgtaacaaggtagccgtaggagaacctgcggctggatcacctcctt |
| Lactobacillus_ | SEQ ID | tatgagagtttgatcctggctcaggatgaacgccggcggtgtgcctaatacatgcaagtcgaacgcgttggcccaattg |
| fermentum | NO: 372 | attgatggtgcttgcacctgattgattaggtcgccaacgagtggcggacgggtgagtaacacgtaggtaacctgccca |
| | | gaagcgggggacaacatttggaaacagatgctaataccgcataacaacgttgttcgcatgaacaacgcttaaaagatg |
| | | gatctcgctatcacttctggatggacctgcgctgcattagcttgaggtggggtaatggcctaccaaggcgatgatgcat |
| | | agccgagttgagagactgatcggccacaatgggactgagacacggcccatactcctacgggaggcagcagtaggga |
| | | atcttccacaatgggcgcaagcctgatggagcaacaccgcgtgagtgaagaagggatcggctcgtaaagctctgagt |
| | | taaagaagaacacgtatgagagtaactgttcatacgttgacggtatttaaccagaaagtcacggctaactacgtgccagc |
| | | agccgcggtaatacgtaggtggcaagcgttatccggatttattgggcgtaaagagagtgcaggcggattctaagtctga |
| | | tgtgaaagccttcggcttaaccggagaagtgcatcggaaactggataacttgagtgcagaagagggtagtggaactcc |
| | | atgtgtagcggtggaatgcgtagatatatggaagaacaccagtggcgaaggcggctacctggtctgcaactgacgag |
| | | agactcgaaagcatgggtagcgaacaggattagataccctggtagtccatgccgtaaacgatgagtgctaggtgttgg |
| | | agggatccgcccttcagtgccggagctaacgcattaagcactccgcctggggagtacgaccgcaaggttgaaactca |
| | | aaggaattgacgggggcccgcacaagcggtggagcatgtggtttaattcgaagctacgcgaagaaccttaccaggtct |
| | | tgacatcttgcgccaaccctagagatagggcgtttccttcgggaacgcaatgacaggtggtgcatggtcgtcgtcagct |
| | | cgtgtcgtgagatgagggttaagtcccgcaacgagcgcaacccagttactagttgccagcattaagttgggcactctag |
| | | tgagactgccggtgacaaaccggaggaaggtggggacgacgtcagatcatcatgccccttatgacctgggctacaca |
| | | cgtgctacaatggacggtacaacgagtcgcgaactcgtgagggcaagcaaatctcttaaaaccgttctcagttcggact |
| | | gcaggctgcaactcgcctgcacgaagtcggaatcgctagtaatcgcggatcagcatgccgcggtgaatacgttcccg |
| | | ggccagtacacaccgcccgtcacaccatgagagtagtaacacccaaagtcggtggggtaaccattaggagccagcc |
| | | gcctaaggtgggacagatgattagggtgaagtcgtaacaaggtagccgtaggagaacctgcggctggatcacctcctt |
| Bacteroides_ | SEQ ID | ctgaaccagccaagtagcgtgaaggatgactgccctatgggagtaaacttctatatatgggaataaagtatccacgtgt |
| thetaiotaomicron | NO: 373 | ggaattttgtatgtaccatatgaataaggatcggctaactccgtgccagcagccgcggtaatacggaggatccgagcgt |
| | | tatccggatttattgggataaagggagcgtaggtggacagttaagtcagagtgaaagtagcggctcaaccgtaaaatt |
| | | gcagttgatactggctgtatgagtacagtagaggtgggcggaattcgtggtgtagcggtgaaatgcttagatatcacga |
| | | agaactccgattgcgaaggcagctcactggactgcaactgacactgatgctcgaaagtgtgggtatcaaacaggatta |
| | | gataccctggtagtccacacagtaaacgatgaatactcgctgtttgcgatatacagtaagcggccaagcgaaagcatta |
| | | agtattccacctggggagtacgccggcaacggtgaaactcaaaggaattgacgggggcccgcacaagcggaggaa |
| | | catgtggtttaattcgatgatacgcgaggaaccttacccgggcttaaattgcatttgaatatattggaaacagtatagccgt |
| | | aaggcaaatgtgaaggtgctgcatggttgtcgtcagctcgtgccgtgaggtgtcggcttaagtgccataacgagcgcaa |
| | | cccttatctttagttactaacaggtcatgctgaggactctagagagactgccgtcgtaagatgtgaggaaggtggggatg |
| | | acgtcaaatcagcacggcccttacgtccggggctacacacgtgttacaatggggggtacagaaggcagctacctggt |
| | | gacaggatgctaatcccaaaagcctctctcagttcggatcgaagtctgcaacccgacttcgtgaagctggattcgctagt |
| | | aatcgcgcatcagccatggcgcggtgaatacgttcccgggccttgtacacaccgcccgtcaagccatgaaagccggg |
| | | ggtacctgaagtacgtaaccgcaaggagcgtcctagggtaaaactggtaattggggctaagtcgtaacaaggtagccg |
| | | taccggaaggtgcggctggaacacctcctt |
| Bacteroides_ | SEQ ID | ctgaaccagccaagtagcgtgaaggatgactgccctatgggagtaaacttatttatatgggaataaagtatccacgtgt |
| thetaiotaomicron | NO: 374 | ggaattttgtatgtaccatatgaataaggatcggctaactccgtgccagcagccgcggtaatacggaggatccgagcgt |
| | | tatccggatttattgggataaagggagcgtaggtggacagttaagtcagagtgaaagtttgcggctcaaccgtaaaatt |
| | | gcagttgatactggctgtatgagtacagtagaggtgggcggaattcgtggtgtagcggtgaaatgcttagatatcacga |
| | | agaactccgattgcgaaggcagctcactggactgcaactgacactgatgctcgaaagtgtgggtatcaaacaggatta |
| | | gataccctggtagtccacacagtaaacgatgaatactcgctgtttgcgatatacagtaagcggccaagcgaaagcatta |
| | | agtattccacctggggagtacgccggcaacggtgaaactcaaaggaattgacgggggcccgcacaagcggaggaa |
| | | catgtggtttaattcgatgatacgcgaggaaccttacccgggcttaaattgcatttgaatatattggaaacagtatagccgt |
| | | aaggcaaatgtgaaggtgctgcatggttgtcgtcagctcgtgccgtgaggtgtcggcttaagtgccataacgagcgcaa |
| | | cccttatctttagttactaacaggtcatgctgaggactctagagagactgccgtcgtaagatgtgaggaaggtggggatg |
| | | acgtcaaatcagcacggcccttacgtccggggctacacacgtgttacaatggggggtacagaaggcagctacctggt |
| | | gacaggatgctaatcccaaaagcctctctcagttcggatcgaagtctgcaacccgacttcgtgaagctggattcgctagt |
| | | aatcgcgcatcagccatggcgcggtgaatacgttcccgggccttgtacacaccgcccgtcaagccatgaaagccggg |
| | | ggtacctgaagtacgtaaccgcaaggagcgtcctagggtaaaactggtaattggggctaagtcgtaacaaggtagccg |
| | | taccggaaggtgcggctggaacacctcctt |
| Bacteroides_ | SEQ ID | atgaagagtttgatcctggctcaggatgaacgctagctacaggcttaacacatgcaagtcgaggggcagcatttcagttt |
| thetaiotaomicron | NO: 375 | gcttgcaaactggagatggcgaccggcgcacgggtgagtaacacgtatccaacctgccgataactcggggatagcct |
| | | ttcgaaagaaagattaatacccgatagtataattagaccgcatggtcttattattaaagaatttcggttatcgatggggatgc |
| | | gttccattaggcagttggtgaggtaacggctcaccaaaccttcgatggataggggttctgagaggaaggtcccccacat |
| | | tggaactgagacacggtccaaactcctacgggaggcagcagtgaggaatattggtcaatggacgagagtctgaacca |
| | | gccaagtagcgtgaaggatgactgccctatgggagtaaacttctatatatgggaataaagattccacgtgtggaattttg |
| | | tatgtaccatatgaataaggatcggctaactccgtgccagcagccgcggtaatacggaggatccgagcgttatccggat |
| | | ttattgggtttaaagggagcgtaggtggacagttaagtcagttgtgaaagtttgcggctcaaccgtaaaattgcagttgat |
| | | actggctgtcttgagtacagtagaggtgggcggaattcgtggtgtagcggtgaaatgcttagatatcacgaagaactcc |
| | | gattgcgaaggcagctcactggactgcaactgacactgatgctcgaaagtgtgggtatcaaacaggattagataccctg |
| | | gtagtccacacagtaaacgatgaatactcgctgtttgcgatatacagtaagcggccaagcgaaagcattaagtattccac |
| | | ctggggagtacgccggcaacggtgaaactcaaaggaattgacgggggcccgcacaagcggaggaacatgtggttta |
| | | attcgatgatacgcgaggaaccttacccgggcttaaattgcatttgaatatattggaaacagtatagccgtaaggcaaatg |
| | | tgaaggtgctgcatggttgtcgtcagctcgtgccgtgaggtgtcggcttaagtgccataacgagcgcaacccttatcttta |
| | | gttactaacaggtcatgctgaggactctagagagactgccgtcgtaagatgtgaggaaggtggggatgacgtcaaatc |
| | | agcacggcccttacgtccggggctacacacgtgttacaatggggggtacagaaggcagctacctggtgacaggatgc |
| | | taatcccaaaagcctctctcagttcggatcgaagtctgcaacccgacttcgtgaagctggattcgctagtaatcgcgcatc |
| | | agccatggcgcggtgaatacgttcccgggccttgtacacaccgcccgtcaagccatgaaagccgggggtacctgaag |
| | | tacgtaaccgcaaggagcgtcctagggtaaaactggtaattggggctaagtcgtaacaaggtagccgtaccggaaggt |
| | | gcggctggaacacctcctt |
| Bacteroides_ | SEQ ID | atgaagagtttgatcctggctcaggatgaacgctagctacaggcttaacacatgcaagtcgaggggcagcatttcagttt |
| thetaiotaomicron | NO: 376 | gcttgcaaactggagatggcgaccggcgcacgggtgagtaacacgtatccaacctgccgataactcggggatagcct |
| | | ttcgaaagaaagattaatacccgatggtataattagaccgcatggtcttgttattaaagaatttcggttatcgatggggatg |
| | | cgttccattaggcagttggtgaggtaacggctcaccaaaccttcgatggataggggttctgagaggaaggtcccccaca |
| | | ttggaactgagacacggtccaaactcctacgggaggcagcagtgaggaatattggtcaatgggcgcaggcctgaacc |
| | | agccaagtagcgtgaaggatgactgccctatgggttgtaaacttcttttatatgggaataaagttttccacgtgtggaatttt |
| | | gtatgtaccatatgaataaggatcggctaactccgtgccagcagccgcggtaatacggaggatccgagcgttatccgg |
| | | atttattgggtttaaagggagcgtaggtggacagttaagtcagttgtgaaagtttgcggctcaaccgtaaaattgcagttg |
| | | atactggctgtcttgagtacagtagaggtgggcggaattcgtggtgtagcggtgaaatgcttagatatcacgaagaactc |
| | | cgattgcgaaggcagctcactggactgcaactgacactgatgctcgaaagtgtgggtatcaaacaggattagataccct |
| | | ggtagtccacacagtaaacgatgaatactcgctgtttgcgatatacagtaagcggccaagcgaaagcattaagtattcca |
| | | cctggggagtacgccggcaacggtgaaactcaaaggaattgacgggggcccgcacaagcggaggaacatgtggttt |
| | | aattcgatgatacgcgaggaaccttacccgggcttaaattgcatttgaatatattggaaacagtatagccgtaaggcaaat |
| | | gtgaaggtgctgcatggttgtcgtcagctcgtgccgtgaggtgtcggcttaagtgccataacgagcgcaacccttatcttt |
| | | agttactaacaggtcatgctgaggactctagagagactgccgtcgtaagatgtgaggaaggtggggatgacgtcaaat |
| | | cagcacggcccttacgtccggggctacacacgtgttacaatggggggtacagaaggcagctacctggtgacaggatg |
| | | ctaatcccaaaagcctctctcagttcggatcgaagtctgcaacccgacttcgtgaagctggattcgctagtaatcgcgcat |
| | | cagccatggcgcggtgaatacgttcccgggccttgtacacaccgcccgtcaagccatgaaagccgggggtacctgaa |
| | | gtacgtaaccgcaaggagcgtcctagggtaaaactggtaattggggctaagtcgtaacaaggtagccgtaccggaag |
| | | gtgcggctggaacacctcctt |
| Lactobacillus_gasseri | SEQ ID | atgagagtttgatcctggctcaggacgaacgctggcggcgtgcctaatacatgcaagtcgagcgagcttgcctagatg |
| | NO: 377 | aatttggtgcttgcaccaaatgaaactagatacaagcgagcggcggacgggtgagtaacacgtgggtaacctgcccaa |
| | | gagactgggataacacctggaaacagatgctaataccggataacaacactagacgcatgtctagagtttaaaagatggt |
| | | tctgctatcactcttggatggacctgcggtgcattagctagttggtaaggtaacggcttaccaaggcaatgatgcatagcc |
| | | gagttgagagactgatcggccacattgggactgagacacggcccaaactcctacgggaggcagcagtagggaatctt |
| | | ccacaatggacgcaagtctgatggagcaacgccgcgtgagtgaagaagggtttcggctcgtaaagctctgttggtagt |
| | | gaagaaagatagaggtagtaactggcctttatttgacggtaattacttagaaagtcacggctaactacgtgccagcagcc |
| | | gcggtaatacgtaggtggcaagcgttgtccggatttattgggcgtaaagcgagtgcaggcggttcaataagtctgatgt |
| | | gaaagccttcggctcaaccggagaattgcatcagaaactgttgaacttgagtgcagaagaggagagtggaactccatg |
| | | tgtagcggtggaatgcgtagatatatggaagaacaccagtggcgaaggcggctctctggtctgcaactgacgctgagg |
| | | ctcgaaagcatgggtagcgaacaggattagataccctggtagtccatgccgtaaacgatgagtgctaagtgttgggag |
| | | gtttccgcctctcagtgctgcagctaacgcattaagcactccgcctggggagtacgaccgcaaggttgaaactcaaagg |
| | | aattgacgggggcccgcacaagcggtggagcatgtggtttaattcgaagcaacgcgaagaaccttaccaggtcttgac |
| | | atccagtgcaaacctaagagattaggagttcccttcggggacgctgagacaggtggtgcatggctgtcgtcagctcgtg |
| | | tcgtgagatgttgggttaagtcccgcaacgagcgcaacccttgtcattagttgccatcattaagttgggcactctaatgag |
| | | actgccggtgacaaaccggaggaaggtggggatgacgtcaagtcatcatgcccatatgacctgggctacacacgtg |
| | | ctacaatggacggtacaacgagaagcgaacctgcgaaggcaagcggatctctgaaagccgttctcagttcggactgta |
| | | ggctgcaactcgcctacacgaagctggaatcgctagtaatcgcggatcagcacgccgcggtgaatacgttcccgggc |
| | | cttgtacacaccgcccgtcacaccatgagagtctgtaacacccaaagccggtgggataacctttataggagtcagccgt |
| | | ctaaggtaggacagatgattagggtgaagtcgtaacaaggtagccgtaggagaacctgcggctggatcacctccttt |
| Lactobacillus_casei_ | SEQ ID | tatgagagtttgatcctggctcaggatgaacgctggcggcgtgcctaatacatgcaagtcgaacgagttctcgttgatga |
| paracasei | NO: 378 | tcggtgcttgcaccgagattcaacatggaacgagtggcggacgggtgagtaacacgtgggtaacctgcccttaagtgg |
| | | gggataacatttggaaacagatgctaataccgcatagatccaagaaccgcatggttcttggctgaaagatggcgtaagc |
| | | tatcgcttttggatggacccgcggcgtattagctagttggtgaggtaatggctcaccaaggcgatgatacgtagccgaac |
| | | tgagaggttgatcggccacattgggactgagacacggcccaaactcctacgggaggcagcagtagggaatcttccac |
| | | aatggacgcaagtctgatggagcaacgccgcgtgagtgaagaaggctttcgggtcgtaaaactctgttgttggagaag |
| | | aatggtcggcagagtaactgttgtcggcgtgacggtatccaaccagaaagccacggctaactacgtgccagcagccg |
| | | cggtaatacgtaggtggcaagcgttatccggatttattgggcgtaaagcgagcgcaggcggttttttaagtctgatgtga |
| | | aagccctcggcttaaccgaggaagcgcatcggaaactgggaaacttgagtgcagaagaggacagtggaactccatgt |
| | | gtagcggtgaaatgcgtagatatatggaagaacaccagtggcgaaggcggctgtctggtctgtaactgacgctgaggc |
| | | tcgaaagcatgggtagcgaacaggattagataccctggtagtccatgccgtaaacgatgaatgctaggtgttggagggt |
| | | ttccgcccttcagtgccgcagctaacgcattaagcattccgcctggggagtacgaccgcaaggttgaaactcaaagga |
| | | attgacgggggcccgcacaagcggtggagcatgtggtttaattcgaagcaacgcgaagaaccttaccaggtcttgaca |
| | | tcttttgatcacctgagagatcaggtttccccttcgggggcaaaatgacaggtggtgcatggttgtcgtcagctcgtgtcg |
| | | tgagatgttgggttaagtcccgcaacgagcgcaacccttatgactagttgccagcatttagttgggcactctagtaagact |
| | | gccggtgacaaaccggaggaaggtggggatgacgtcaaatcatcatgccccttatgacctgggctacacacgtgctac |
| | | aatggatggtacaacgagttgcgagaccgcgaggtcaagctaatctcttaaagccattctcagttcggactgtaggctgc |
| | | aactcgcctacacgaagtcggaatcgctagtaatcgcggatcagcacgccgcggtgaatacgttcccgggccttgtac |
| | | acaccgcccgtcacaccatgagagtttgtaacacccgaagccggtggcgtaacccttttagggagcgagccgtctaag |
| | | gtgggacaaatgattagggtgaagtcgtaacaaggtagccgtaggagaacctgcggctggatcacctcctt |
| Lactobacillus_casei_ | SEQ ID | tatgagagatgatcctggctcaggatgaacgctggcggcgtgcctaatacatgcaagtcgaacgagactcgttgatga |
| paracasei | NO: 379 | tcggtgcttgcaccgagattcaacatggaacgagtggcggacgggtgagtaacacgtgggtaacctgcccttaagtgg |
| | | gggataacataggaaacagatgctaataccgcatagatccaagaaccgcatggacttggctgaaagatggcgtaagc |
| | | tatcgcttttggatggacccgcggcgtattagctagttggtgaggtaatggctcaccaaggcgatgatacgtagccgaac |
| | | tgagaggttgatcggccacattgggactgagacacggcccaaactcctacgggaggcagcagtagggaatcttccac |
| | | aatggacgcaagtctgatggagcaacgccgcgtgagtgaagaaggattcgggtcgtaaaactctgagttggagaag |
| | | aatggtcggcagagtaactgagtcggcgtgacggtatccaaccagaaagccacggctaactacgtgccagcagccg |
| | | cggtaatacgtaggtggcaagcgttatccggatttattgggcgtaaagcgagcgcaggcggttttttaagtctgatgtga |
| | | aagccctcggcttaaccgaggaagcgcatcggaaactgggaaacttgagtgcagaagaggacagtggaactccatgt |
| | | gtagcggtgaaatgcgtagatatatggaagaacaccagtggcgaaggcggctgtctggtctgtaactgacgctgaggc |
| | | tcgaaagcatgggtagcgaacaggattagataccctggtagtccatgccgtaaacgatgaatgctaggtgttggagggt |
| | | ttccgcccttcagtgccgcagctaacgcattaagcattccgcctggggagtacgaccgcaaggttgaaactcaaagga |
| | | attgacgggggcccgcacaagcggtggagcatgtggtttaattcgaagcaacgcgaagaaccttaccaggtcttgaca |
| | | tcttttgatcacctgagagatcaggatccccttcgggggcaaaatgacaggtggtgcatggttgtcgtcagctcgtgtcg |
| | | tgagatgagggttaagtcccgcaacgagcgcaacccttatgactagttgccagcatttagttgggcactctagtaagact |
| | | gccggtgacaaaccggaggaaggtggggatgacgtcaaatcatcatgccccttatgacctgggctacacacgtgctac |
| | | aatggatggtacaacgagttgcgagaccgcgaggtcaagctaatctcttaaagccattctcagttcggactgtaggctgc |
| | | aactcgcctacacgaagtcggaatcgctagtaatcgcggatcagcacgccgcggtgaatacgttcccgggccttgtac |
| | | acaccgcccgtcacaccatgagagtttgtaacacccgaagccggtggcgtaacccttttagggagcgagccgtctaag |
| | | gtgggacaaatgattagggtgaagtcgtaacaaggtagccgtaggagaacctgcggctggatcacctcctt |
| Enterococcus_faecium | SEQ ID | tatgagagatgatcctggctcaggacgaacgctggcggcgtgcctaatacatgcaagtcgaacgctattctttcaccgg |
| | NO: 380 | agcttgctccaccgaaagaaaaagagtggcgaacgggtgagtaacacgtgggtaacctgcccatcagaaggggata |
| | | acacttggaaacaggtgctaataccgtataacactattttccgcatggaagaaagttgaaaggcgcttttgcgtcactgat |
| | | ggatggacccgcggtgcattagctagttggtgaggtaacggctcaccaaggcaacgatgcatagccgacctgagagg |
| | | gtgatcggccacactgggactgagacacggcccagactcctacgggaggcagcagtagggaatcttcggcaatgga |
| | | cgaaagtctgaccgagcaacgccgcgtgagtgaagaaggttttcggatcgtaaaactctgttgttagagaagaacaag |
| | | gatgagagtagaacgttcatcccttgacggtatctaaccagaaagccacggctaactacgtgccagcagccgcggtaa |
| | | tacgtaggtggcaagcgttgtccggatttattgggcgtaaagcgagcgcaggcggtttcttaagtctgatgtgaaagccc |
| | | ccggctcaaccggggagggtcattggaaactgggagacttgagtgcagaagaggagagtggaattccatgtgtagcg |
| | | gtgaaatgcgtagatatatggaggaacaccagtggcgaaggcggctctctggtctgtaactgacgctgaggctcgaaa |
| | | gcgtggggagcgaacaggattagataccctggtagtccacgccgtaaacgatgagtgctaagtgttggagggtttccg |
| | | cccttcagtgctgcagcaaacgcattaagcactccgcctggggagtacgaccgcaaggttgaaactcaaaggaattga |
| | | cgggggcccgcacaagcggtggagcatgtggtttaattcgaagcaacgcgaagaaccttaccaggtcttgacatccat |
| | | gaccactctagagatagagcttccccttcgggggcaaagtgacaggtggtgcatggagtcgtcagctcgtgtcgtgag |
| | | atgttgggttaagtcccgcaacgagcgcaacccttattgttagttgccatcatttagttgggcactctagcgagactgccg |
| | | gtgacaaaccggaggaaggtggggatgacgtcaaatcatcatgccccttatgacctgggctacacacgtgctacaatg |
| | | ggaagtacaacgagttgcgaagtcgcgaggctaagctaatctcttaaagcttctctcagttcggattgtaggctgcaact |
| | | cgcctacatgaagccggaatcgctagtaatcgcggatcagcacgccgcggtgaatacgttcccgggccttgtacacac |
| | | cgcccgtcacaccacgagagtagtaacacccgaagtcggtgaggtaaccatttggagccagccgcctaaggtggga |
| | | tagatgattggggtgaagtcgtaacaaggtagccgtatcggaaggtgcggctggatcacctccat |
| Lactobacillus_casei_ | SEQ ID | tatgagagatgatcctggctcaggatgaacgctggcggcgtgcctaatacatgcaagtcgaacgagactcgttgatga |
| paracasei | NO: 381 | tcggtgcttgcaccgagattcaacatggaacgagtggcggacgggtgagtaacacgtgggtaacctgcccttaagtgg |
| | | gggataacataggaaacagatgctaataccgcatagatccaagaaccgcatggacttggctgaaagatggcgtaagc |
| | | tatcgcttttggatggacccgcggcgtattagctagttggtgaggtaatggctcaccaaggcgatgatacgtagccgaac |
| | | tgagaggttgatcggccacattgggactgagacacggcccaaactcctacgggaggcagcagtagggaatcttccac |
| | | aatggacgcaagtctgatggagcaacgccgcgtgagtgaagaaggattcgggtcgtaaaactctgagttggagaag |
| | | aatggtcggcagagtaactgagtcggcgtgacggtatccaaccagaaagccacggctaactacgtgccagcagccg |
| | | cggtaatacgtaggtggcaagcgttatccggatttattgggcgtaaagcgagcgcaggcggttttttaagtctgatgtga |
| | | aagccctcggcttaaccgaggaagcgcatcggaaactgggaaacttgagtgcagaagaggacagtggaactccatgt |
| | | gtagcggtgaaatgcgtagatatatggaagaacaccagtggcgaaggcggctgtctggtctgtaactgacgctgaggc |
| | | tcgaaagcatgggtagcgaacaggattagataccctggtagtccatgccgtaaacgatgaatgctaggtgttggagggt |
| | | ttccgcccttcagtgccgcagctaacgcattaagcattccgcctggggagtacgaccgcaaggttgaaactcaaagga |
| | | attgacgggggcccgcacaagcggtggagcatgtggtttaattcgaagcaacgcgaagaaccttaccaggtcttgaca |
| | | tcttttgatcacctgagagatcaggatccccttcgggggcaaaatgacaggtggtgcatggttgtcgtcagctcgtgtcg |
| | | tgagatgagggttaagtcccgcaacgagcgcaacccttatgactagttgccagcatttagagggcactctagtaagact |
| | | gccggtgacaaaccggaggaaggtggggatgacgtcaaatcatcatgccccttatgacctgggctacacacgtgctac |
| | | aatggatggtacaacgagttgcgagaccgcgaggtcaagctaatctcttaaagccattctcagttcggactgtaggctgc |
| | | aactcgcctacacgaagtcggaatcgctagtaatcgcggatcagcacgccgcggtgaatacgttcccgggccttgtac |
| | | acaccgcccgtcacaccatgagagtttgtaacacccgaagccggtggcgtaacccttttagggagcgagccgtctaag |
| | | gtgggacaaatgattagggtgaagtcgtaacaaggtagccgtaggagaacctgcggctggatcacctcctt |
| Lactobacillus_casei_ | SEQ ID | tatgagagatgatcaggctcaggatgaacgctggcggcgtgcctaatacatgcaagtcgaacgagttctcgttgatga |
| paracasei | NO: 382 | tcggtgcttgcaccgagattcaacatggaacgagtggcggacgggtgagtaacacgtgggtaacctgcccttaagtgg |
| | | gggataacatttggaaacagatgctaataccgcatagatccaagaaccgcatggacttggctgaaagatggcgtaagc |
| | | tatcgcttttggatggacccgcggcgtattagctagttggtgaggtaacggctcaccaaggcgatgatacgtagccgaa |
| | | ctgagaggttgatcggccacattgggactgagacacggcccaaactcctacgggaggcagcagtagggaatcttcca |
| | | caatggacgcaagtctgatggagcaacgccgcgtgagtgaagaaggctttcgggtcgtaaaactctgagttggagaa |
| | | gaatggtcggcagagtaactgttgtcggcgtgacggtatccaaccagaaagccacggctaactacgtgccagcagcc |
| | | gcggtaatacgtaggtggcaagcgttatccggatttattgggcgtaaagcgagcgcaggcggttttttaagtctgatgtg |
| | | aaagccctcggcttaaccgaggaagcgcatcggaaactgggaaacttgagtgcagaagaggacagtggaactccat |
| | | gtgtagcggtgaaatgcgtagatatatggaagaacaccagtggcgaaggcggctgtctggtctgtaactgacgctgag |
| | | gctcgaaagcatgggtagcgaacaggattagataccctggtagtccatgccgtaaacgatgaatgctaggtgttggag |
| | | ggtttccgcccttcagtgccgcagctaacgcattaagcattccgcctggggagtacgaccgcaaggttgaaactcaaag |
| | | gaattgacgggggcccgcacaagcggtggagcatgtggtttaattcgaagcaacgcgaagaaccttaccaggtcttga |
| | | catcttttgatcacctgagagatcaggtttccccttcgggggcaaaatgacaggtggtgcatggttgtcgtcagctcgtgt |
| | | cgtgagatgagggttaagtcccgcaacgagcgcaacccttatgactagagccagcatttagttgggcactctagtaag |
| | | actgccggtgacaaaccggaggaaggtggggatgacgtcaaatcatcatgccccttatgacctgggctacacacgtgc |
| | | tacaatggatggtacaacgagttgcgagaccgcgaggttaagctaatctcttaaagccattctcagttcggactgtaggc |
| | | tgcaactcgcctacacgaagtcggaatcgctagtaatcgcggatcagcacgccgcggtgaatacgttcccgggccttg |
| | | tacacaccgcccgtcacaccatgagagtagtaacacccgaagccggtggcgtaacccattagggagcgagccgtct |
| | | aaggtgggacaaatgattagggtgaagtcgtaacaaggtagccgtaggagaacctgcggctggatcacctcctt |
| Lactobacillus_casei_ | SEQ ID | tatgagagatgatcctggctcaggatgaacgctggcggcgtgcctaatacatgcaagtcgaacgagttctcgttgatga |
| paracasei | NO: 383 | tcggtgcttgcactgagattcaacatggaacgagtggcggacgggtgagtaacacgtgggtaacctgcccttaagtgg |
| | | gggataacataggaaacagatgctaataccgcatagatccaagaaccgcatggacttggctgaaagatggcgtaagc |
| | | tatcgcttttggatggacccgcggcgtattagctagttggtgaggtaatggctcaccaaggcgatgatacgtagccgaac |
| | | tgagaggttgatcggccacattgggactgagacacggcccaaactcctacgggaggcagcagtagggaatcttccac |
| | | aatggacgcaagtctgatggagcaacgccgcgtgagtgaagaaggctttcgggtcgtaaaactctgttgttggagaag |
| | | aatggtcggcagagtaactgttgtcggcgtgacggtatccaaccagaaagccacggctaactacgtgccagcagccg |
| | | cggtaatacgtaggtggcaagcgttatccggatttattgggcgtaaagcgagcgcaggcggttttttaagtctgatgtga |
| | | aagccctcggcttaaccgaggaagcgcatcggaaactgggaaacttgagtgcagaagaggacagtggaactccatgt |
| | | gtagcggtgaaatgcgtagatatatggaagaacaccagtggcgaaggcggctgtctggtctgtaactgacgctgaggc |
| | | tcgaaagcatgggtagcgaacaggattagataccctggtagtccatgccgtaaacgatgaatgctaggtgttggagggt |
| | | ttccgcccttcagtgccgcagctaacgcattaagcattccgcctggggagtacgaccgcaaggttgaaactcaaagga |
| | | attgacgggggcccgcacaagcggtggagcatgtggtttaattcgaagcaacgcgaagaaccttaccaggtcttgaca |
| | | tcttttgatcacctgagagatcaggtaccccttcgggggcaaaatgacaggtggtgcatggttgtcgtcagctcgtgtcg |
| | | tgagatgagggttaagtcccgcaacgagcgcaacccttatgactagttgccagcatttagttgggcactctagtaagact |
| | | gccggtgacaaaccggaggaaggtggggatgacgtcaaatcatcatgccccttatgacctgggctacacacgtgctac |
| | | aatggatggtacaacgagttgcgagaccgcgaggtcaagctaatctcttaaagccattctcagttcggactgtaggctgc |
| | | aactcgcctacacgaagtcggaatcgctagtaatcgcggatcagcacgccgcggtgaatacgttcccgggccttgtac |
| | | acaccgcccgtcacaccatgagagtttgtaacacccgaagccggtggcgtaacccttttagggagcgagccgtctaag |
| | | gtgggacaaatgattagggtgaagtcgtaacaaggtagccgtaggagaacctgcggctggatcacctcctt |
| Lactobacillus_casei_ | SEQ ID | tatgagagatgatcctggctcaggatgaacgctggcggcgtgcctaatacatgcaagtcgaacgagttctcgttgatga |
| paracasei | NO: 384 | tcggtgcttgcaccgagattcaacatggaacgagtggcggacgggtgagtaacacgtgggtaacctgcccttaagtgg |
| | | gggataacatttggaaacagatgctaataccgcatagatccaagaaccgcatggttcaggctgaaagatggcgtaagc |
| | | tatcgcttttggatggacccgcggcgtattagctagttggtgaggtaatggctcaccaaggcgatgatacgtagccgaac |
| | | tgagaggttgatcggccacattgggactgagacacggcccaaactcctacgggaggcagcagtagggaatcttccac |
| | | aatggacgcaagtctgatggagcaacgccgcgtgagtgaagaaggctttcgggtcgtaaaactctgagttggagaag |
| | | aatggtcggcagagtaactgttgtcggcgtgacggtatccaaccagaaagccacggctaactacgtgccagcagccg |
| | | cggtaatacgtaggtggcaagcgttatccggatttattgggcgtaaagcgagcgcaggcggttttttaagtctgatgtga |
| | | aagccctcggcttaaccgaggaagcgcatcggaaactgggaaacttgagtgcagaagaggacagtggaactccatgt |
| | | gtagcggtgaaatgcgtagatatatggaagaacaccagtggcgaaggcggctgtctggtctgtaactgacgctgaggc |
| | | tcgaaagcatgggtagcgaacaggattagataccctggtagtccatgccgtaaacgatgaatgctaggtgttggagggt |
| | | ttccgcccttcagtgccgcagctaacgcattaagcattccgcctggggagtacgaccgcaaggttgaaactcaaagga |
| | | attgacgggggcccgcacaagcggtggagcatgtggtttaattcgaagcaacgcgaagaaccttaccaggtcttgaca |
| | | tcttttgatcacctgagagatcaggtttccccttcgggggcaaaatgacaggtggtgcatggttgtcgtcagctcgtgtcg |
| | | tgagatgttgggttaagtcccgcaacgagcgcaacccttatgactagttgccagcatttagttgggcactctagtaagact |
| | | gccggtgacaaaccggaggaaggtggggatgacgtcaaatcatcatgccccttatgacctgggctacacacgtgctac |
| | | aatggatggtacaacgagttgcgagaccgcgaggtcaagctaatctcttaaagccattctcagttcggactgtaggctgc |
| | | aactcgcctacacgaagtcggaatcgctagtaatcgcggatcagcacgccgcggtgaatacgttcccgggccttgtac |
| | | acaccgcccgtcacaccatgagagtttgtaacacccgaagccggtggcgtaacccttttagggagcgagccgtctaag |
| | | gtgggacaaatgattagggtgaagtcgtaacaaggtagccgtaggagaacctgcggctggatcacctcctt |
| Lactobacillus_casei_ | SEQ ID | tatgagagtttgatcaggctcaggatgaacgctggcggcgtgcctaatacatgcaagtcgaacgagttctcgttgatga |
| paracasei | NO: 385 | tcggtgcttgcaccgagattcaacatagaacgagtggcggacgggtgagtaacacgtgggtaacctgcccttaagtgg |
| | | gggataacatttggaaacagatgctaataccgcatagatccaagaaccgcatggacctggctgaaagatggcgtaagc |
| | | tatcgcttttggatggacccgcggcgtattagctagttggtgaggtaatggctcaccaaggcgatgatacgtagccgaac |
| | | tgagaggttgatcggccacattgggactgagacacggcccaaactcctacgggaggcagcagtagggaatcttccac |
| | | aatggacgcaagtctgatggagcaacgccgcgtgagtgaagaaggctttcgggtcgtaaaactctgagttggagaag |
| | | aatggtcggcagagtaactgttgtcggcgtgacggtatccaaccagaaagccacggctaactacgtgccagcagccg |
| | | cggtaatacgtaggtggcaagcgttatccggatttattgggcgtaaagcgagcgcaggcggttttttaagtctgatgtga |
| | | aagccctcggcttaaccgaggaagcgcatcggaaactgggaaacttgagtgcagaagaggacagtggaactccatgt |
| | | gtagcggtgaaatgcgtagatatatggaagaacaccagtggcgaaggcggctgtctggtctgtaactgacgctgaggc |
| | | tcgaaagcatgggtagcgaacaggattagataccctggtagtccatgccgtaaacgatgaatgctaggtgttggagggt |
| | | ttccgccatcagtgccgcagctaacgcattaagcattccgcctggggagtacgaccgcaaggttgaaactcaaagga |
| | | attgacgggggcccgcacaagcggtggagcatgtggtttaattcgaagcaacgcgaagaaccttaccaggtcttgaca |
| | | tcttttgatcacctgagagatcaggtttccccttcgggggcaaaatgacaggtggtgcatggttgtcgtcagctcgtgtcg |
| | | tgagatgttgggttaagtcccgcaacgagcgcaacccttatgactagttgccagcatttagttgggcactctagtaagact |
| | | gccggtgacaaaccggaggaaggtggggatgacgtcaaatcatcatgccccttatgacctgggctacacacgtgctac |
| | | aatggatggtacaacgagttgcgagaccgcgaggtcaagctaatctcttaaagccattctcagttcggactgtaggctgc |
| | | aactcgcctacacgaagtcggaatcgctagtaatcgcggatcagcacgccgcggtgaatacgttcccgggccttgtac |
| | | acaccgcccgtcacaccatgagagtttgtaacacccgaagccggtggcgtaacccttttagggagcgagccgtctaag |
| | | gtgggacaaatgattagggtgaagtcgtaacaaggtagccgtaggagaacctgcggctggatcacctcatt |
| Lactobacillus_ | SEQ ID | tttgagagtttgatcctggctcaggacgaacgctggcggcgtgcctaatacatgcaagtcgaacgaactctggtattgatt |
| plantarum | NO: 386 | ggtgcttgcatcatgatttacatttgagtgagtggcgaactggtgagtaacacgtgggaaacctgcccagaagcgggg |
| | | gataacacctggaaacagatgctaataccgcataacaacttggaccgcatggtccgagtttgaaagatggcttcggctat |
| | | cacttctggatggtcccgcggcgtattagctagatggtggggtaacggctcaccatggcaatgatacgtagccgacctg |
| | | agagggtaatcggccacattgggactgagacacggcccaaactcctacgggaggcagcagtagggaatcttccaca |
| | | atggacgaaagtctgatggagcaacgccgcgtgagtgaagaagggtttcggctcgtaaaactctgttgttaaagaaga |
| | | acatatctgagagtaactgttcaggtattgacggtatttaaccagaaagccacggctaactacgtgccagcagccgcgg |
| | | taatacgtaggtggcaagcgttgtccggatttattgggcgtaaagcgagcgcaggcggttttttaagtctgatgtgaaag |
| | | ccttcggctcaaccgaagaagtgcatcggaaactgggaaacttgagtgcagaagaggacagtggaactccatgtgta |
| | | gcggtgaaatgcgtagatatatggaagaacaccagtggcgaaggcggctgtctggtctgtaactgacgctgaggctcg |
| | | aaagtatgggtagcaaacaggattagataccctggtagtccataccgtaaacgatgaatgctaagtgttggagggtttcc |
| | | gcccttcagtgctgcagctaacgcattaagcattccgcctggggagtacggccgcaaggctgaaactcaaaggaattg |
| | | acgggggcccgcacaagcggtggagcatgtggataattcgaagctacgcgaagaaccttaccaggtcttgacatact |
| | | atgcaaatctaagagattagacgacccttcggggacatggatacaggtggtgcatggagtcgtcagctcgtgtcgtga |
| | | gatgttgggttaagtcccgcaacgagcgcaacccttattatcagttgccagcattaagttgggcactctggtgagactgc |
| | | cggtgacaaaccggaggaaggtggggatgacgtcaaatcatcatgccccttatgacctgggctacacacgtgctacaa |
| | | tggatggtacaacgagttgcgaactcgcgagagcaagctaatctcttaaagccattctcagttcggattgtaggctgcaa |
| | | ctcgcctacatgaagtcggaatcgctagtaatcgcggatcagcatgccgcggtgaatacgttcccgggccttgtacaca |
| | | ccgcccgtcacaccatgagagtttgtaacacccaaagtcggtggggtaaccttttaggaaccagccgcctaaggtggg |
| | | acagatgattagggtgaagtcgtaacaaggtagccgtaggagaacctgcggctggatcacctcctt |
| Enterobacter_cloacae | SEQ ID | agcggcggacgggtgagtaatgtctgggaaactgcctgatggagggggataactactggaaacggtagctaataccg |
| | NO: 387 | cataacgtcgcaagaccaaagagggggaccttcgggcctcttgccatcagatgtgcccagatgggattagctagtagg |
| | | tggggtaacggctcacctaggcgacgatccctagctggtctgagaggatgaccagccacactggaactgagacacgg |
| | | tccagactcctacgggaggcagcagtggggaatattgcacaatgggcgcaagcctgatgcagccatgccgcgtgtat |
| | | gaagaaggccttcgggttgtaaagtactttcagcggggaggaaggcgttgaggttaataacctcagcgattgacgttac |
| | | ccgcagaagaagcaccggctaactccgtgccagcagccgcggtaatacggagggtgcaagcgttaatcggaattact |
| | | gggcgtaaagcgcacgcaggcggtctgtcaagtcggatgtgaaatccccgggctcaacctgggaactgcattcgaaa |
| | | ctggcaggctagagtcttgtagaggggggtagaattccaggtgtagcggtgaaatgcgtagagatctggaggaatacc |
| | | ggtggcgaaggcggccccctggacaaagactgacgctcaggtgcgaaagcgtggggagcaaacaggattagatac |
| | | cctggtagtccacgccgtaaacgatgtcgacttggaggagtgcccttgaggcgtggcaccggagctaacgcgttaag |
| | | tcgaccgcctggggagtacggccgcaaggttaaaactcaaatgaattgacgggggcccgcacaagcggtggagcat |
| | | gtggtttaattcgatgcaacgcgaagaaccttacctactcttgacatccagagaactaccagagatggattggtgccttc |
| | | gggaactctgagacaggtgctgcatggctgtcgtcagctcgtgagtgaaatgttgggttaagtcccgcaacgagcgca |
| | | acccttatcctttgttgccagcggttcggccgggaactcaaaggagactgccagtgataaactggaggaaggtgggga |
| | | tgacgtcaagtcatcatggcccttacgagtagggctacacacgtgctacaatggcgcatacaaagagaagcgacctcg |
| | | cgagagcaagcggacctcataaagtgcgtcgtagtccggattggagtctgcaactcgactccatgaagtcggaatcgc |
| | | tagtaatcgtagatcagaatgctacggtgaatacgttcccgggccttgtacacaccgcccgtcacaccatgggagtggg |
| | | ttgcaaaagaagtaggtagcttaaccttcgggagggcgcttaccactttgtgattcatgactggggtgaagtcgtaacaa |
| | | ggtaaccgtaggggaacctgcggttggatcacctcctt |
| Escherichia_coli | SEQ ID | ttgaagagtttgatcatggctcagattgaacgctggcggcaggcctaacacatgcaagtcgaacggtaacaggaatca |
| | NO: 388 | gcttgctgattcgctgacgagtggcggacgggtgagtaatgtctgggaaactgcctgatggagggggataactactgg |
| | | aaacggtagctaataccgcataacgtcgcaagaccaaagagggggaccttcgggcctcttgccatcggatgtgccca |
| | | gatgggattagctagtaggtggggtaaaggctcacctaggcgacgatccctagctggtctgagaggatgaccagcca |
| | | cactggaactgagacacggtccagactcctacgggaggcagcagtggggaatattgcacaatgggcgcaagcctgat |
| | | gcagccatgccgcgtgtatgaagaaggccttcgggagtaaagtactttcagcggggaggaagggagtaaagttaata |
| | | cctttgctcattgacgttacccgcagaagaagcaccggctaactccgtgccagcagccgcggtaatacggagggtgca |
| | | agcgttaatcggaattactgggcgtaaagcgcacgcaggcggtttgttaagtcagatgtgaaatccccgggctcaacct |
| | | gggaactgcatctgatactggcaagcttgagtctcgtagaggggggtagaattccaggtgtagcggtgaaatgcgtag |
| | | agatctggaggaataccggtggcgaaggcggccccctggacgaagactgacgctcaggtgcgaaagcgtggggag |
| | | caaacaggattagataccctggtagtccacgccgtaaacgatgtcgacttggaggttgtgcccttgaggcgtggcttcc |
| | | ggagctaacgcgttaagtcgaccgcctggggagtacggccgcaaggttaaaactcaaatgaattgacgggggcccg |
| | | cacaagcggtggagcatgtggtttaattcgatgcaacgcgaagaaccttacctggtcttgacatccacagaaccagtag |
| | | agatacgagggtgccttcgggaactgtgagacaggtgctgcatggctgtcgtcagctcgtgttgtgaaatgttgggttaa |
| | | gtcccgcaacgagcgcaacccttatcctttgttgccagcggtccggccgggaactcaaaggagactgccagtgataaa |
| | | ctggaggaaggtggggatgacgtcaagtcatcatggcccttacgaccagggctacacacgtgctacaatggcgcata |
| | | caaagagaagcgacctcgcgagagcaagcggacctcataaagtgcgtcgtagtccggattggagtctgcaactcgac |
| | | tccatgaagtcggaatcgctagtaatcgtggatcagaatgccacggtgaatacgttcccgggccttgtacacaccgccc |
| | | gtcacaccatgggagtgggttgcaaaagaagtaggtagcttaaccttcgggagggcgcttaccactttgtgattcatgac |
| | | tggggtgaagtcgtaacaaggtaaccgtaggggaacctgcggttggatcacctcctt |
| Escherichia_coli | SEQ ID | ttgaagagtttgatcatggctcagattgaacgctggcggcaggcctaacacatgcaagtcgaacggtaacaggaagaa |
| | NO: 389 | gcttgcttcatgctgacgagtggcggacgggtgagtaatgtctgggaaactgcctgatggagggggataactactgga |
| | | aacggtagctaataccgcataacgtcgcaagaccaaagagggggaccacgggcctcttgccatcggatgtgcccag |
| | | atgggattagctagtaggtggggtaaaggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtatgaagaaggccttcgggttgtaaagtactttcagcggggaggaagggagtaaagttaatac |
| | | ctttgctcattgacgttacccgcagaagaagcaccggctaactccgtgccagcagccgcggtaatacggagggtgcaa |
| | | gcgttaatcggaattactgggcgtaaagcgcacgcaggcggtttgttaagtcagatgtgaaatccccgggctcaacctg |
| | | ggaactgcatctgatactggcaagcttgagtctcgtagaggggggtagaattccaggtgtagcggtgaaatgcgtaga |
| | | gatctggaggaataccggtggcgaaggcggccccctggacgaagactgacgctcaggtgcgaaagcgtggggagc |
| | | aaacaggattagataccctggtagtccacgccgtaaacgatgtcgacttggaggttgtgcccttgaggcgtggcttccg |
| | | gagctaacgcgttaagtcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaactttccagag |
| | | atggattggtgccttcgggaactgtgagacaggtgctgcatggctgtcgtcagctcgtgttgtgaaatgttgggttaagtc |
| | | ccgcaacgagcgcaacccttatcctagttgccagcggtccggccgggaactcaaaggagactgccagtgataaactg |
| | | gaggaaggtggggatgacgtcaagtcatcatggccatacgaccagggctacacacgtgctacaatggcgcatacaa |
| | | agagaagcgacctcgcgagagcaagcggacctcataaagtgcgtcgtagtccggattggagtctgcaactcgactcc |
| | | atgaagtcggaatcgctagtaatcgtggatcagaatgccacggtgaatacgacccgggccagtacacaccgcccgtc |
| | | acaccatgggagtgggttgcaaaagaagtaggtagcttaaccttcgggagggcgcttaccactttgtgattcatgactgg |
| | | ggtgaagtcgtaacaaggtaaccgtaggggaacctgcggttggatcacctcctt |
| Escherichia_coli | SEQ ID | ttgaagagtttgatcatggctcagattgaacgctggcggcaggcctaacacatgcaagtcgaacggtaacaggaagaa |
| | NO: 390 | gcttgcttcatgctgacgagtggcggacgggtgagtaatgtagggaaactgcctgatggagggggataactactgga |
| | | aacggtagctaataccgcataacgtcgcaagaccaaagagggggaccttcgggcctcttgccatcggatgtgcccag |
| | | atgggattagctagtaggtggggtaaaggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtatgaagaaggccttcgggagtaaagtactttcagcggggaggaagggagtaaagttaatac |
| | | ctttgctcattgacgttacccgcagaagaagcaccggctaactccgtgccagcagccgcggtaatacggagggtgcaa |
| | | gcgttaatcggaattactgggcgtaaagcgcacgcaggcggtttgttaagtcagatgtgaaatccccgggctcaacctg |
| | | ggaactgcatctgatactggcaagcttgagtctcgtagaggggggtagaattccaggtgtagcggtgaaatgcgtaga |
| | | gatctggaggaataccggtggcgaaggcggccccctggacgaagactgacgctcaggtgcgaaagcgtggggagc |
| | | aaacaggattagataccctggtagtccacgccgtaaacgatgtcgacttggaggttgtgcccttgaggcgtggcttccg |
| | | gagctaacgcgttaagtcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaactttccagag |
| | | atggattggtgccttcgggaactgtgagacaggtgctgcatggctgtcgtcagctcgtgttgtgaaatgttgggttaagtc |
| | | ccgcaacgagcgcaacccttatcctagttgccagcggtccggccgggaactcaaaggagactgccagtgataaactg |
| | | gaggaaggtggggatgacgtcaagtcatcatggcccttacgaccagggctacacacgtgctacaatggcgcatacaa |
| | | agagaagcgacctcgcgagagcaagcggacctcataaagtgcgtcgtagtccggattggagtctgcaactcgactcc |
| | | atgaagtcggaatcgctagtaatcgtggatcagaatgccacggtgaatacgacccgggccagtacacaccgcccgtc |
| | | acaccatgggagtgggttgcaaaagaagtaggtagcttaaccttcgggagggcgcttaccactttgtgattcatgactgg |
| | | ggtgaagtcgtaacaaggtaaccgtaggggaacctgcggttggatcacctcctt |
| Escherichia_coli | SEQ ID | ttgaagagtttgatcatggctcagattgaacgctggcggcaggcctaacacatgcaagtcgaacggtaacaggaagaa |
| | NO: 391 | gcttgcttcatgctgacgagtggcggacgggtgagtaatgtagggaaactgcctgatggagggggataactactgga |
| | | aacggtagctaataccgcataacgtcgcaagaccaaagagggggaccttcgggcctcttgccatcggatgtgcccag |
| | | atgggattagctagtaggtggggtaaaggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtatgaagaaggccttcgggttgtaaagtactttcagcggggaggaagggagtaaagttaatac |
| | | ctttgctcattgacgttacccgcagaagaagcaccggctaactccgtgccagcagccgcggtaatacggagggtgcaa |
| | | gcgttaatcggaattactgggcgtaaagcgcacgcaggcggtttgttaagtcagatgtgaaatccccgggctcaacctg |
| | | ggaactgcatctgatactggcaagcttgagtctcgtagaggggggtagaattccaggtgtagcggtgaaatgcgtaga |
| | | gatctggaggaataccggtggcgaaggcggccccctggacgaagactgacgctcaggtgcgaaagcgtggggagc |
| | | aaacaggattagataccctggtagtccacgccgtaaacgatgtcgacttggaggttgtgcccttgaggcgtggcttccg |
| | | gagctaacgcgttaagtcgaccgcctggggagtacggccgcaaggttaaaactcaaatgaattgacgggggcccgca |
| | | caagcggtggagcatgtggataattcgatgcaacgcgaagaaccttacctggtcttgacatccacagaactaccagag |
| | | atggattggtgccttcgggaactgtgagacaggtgctgcatggctgtcgtcagctcgtgttgtgaaatgagggttaagtc |
| | | ccgcaacgagcgcaacccttatcctagttgccagcggtccggccgggaactcaaaggagactgccagtgataaactg |
| | | gaggaaggtggggatgacgtcaagtcatcatggccatacgaccagggctacacacgtgctacaatggcgcatacaa |
| | | agagaagcgacctcgcgagagcaagcggacctcataaagtgcgtcgtagtccggattggagtctgcaactcgactcc |
| | | aatgaagtcggaatcgctagtaatcgtggatcagaatgccacggtgatacgttcccgggccttgtacacaccgcccgtc |
| | | acaccatgggagtgggttgcaaaagaagtaggtagcttaaccttcgggagggcgcttaccactttgtgattcatgactgg |
| | | ggtgaagtcgtaacaaggtaaccgtaggggaacctgcggttggatcacctcctt |
| Escherichia_coli | SEQ ID | ttgaagagtttgatcatggctcagattgaacgctggcggcaggcctaacacatgcaagtcgaacggtaacaggaagaa |
| | NO: 392 | gcttgcttctttgctgacgagtggcggacgggtgagtaatgtctgggaaactgcctgatggagggggataactactgga |
| | | aacggtagctaataccgcataacgtcgcaagaccaaagagggggaccttcgggcctcttgccatcggatgtgcccag |
| | | atgggattagctagtaggtggggtaaaggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtatgaagaaggccttcgggttgtaaagtactttcagcggggaggaagggagtaaagttaatac |
| | | ctttgctcattgacgttacccgcagaagaagcaccggctaactccgtgccagcagccgcggtaatacggagggtgcaa |
| | | gcgttaatcggaattactgggcgtaaagcgcacgcaggcggtttgttaagtcagatgtgaaatccccgggctcaacctg |
| | | ggaactgcatctgatactggcaagatgagtctcgtagaggggggtagaattccaggtgtagcggtgaaatgcgtaga |
| | | gatctggaggaataccggtggcgaaggcggccccctggacgaagactgacgctcaggtgcgaaagcgtggggagc |
| | | aaacaggattagataccctggtagtccacgccgtaaacgatgtcgacttggaggttgtgcccttgaggcgtggcttccg |
| | | gagctaacgcgttaagtcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaactttccagag |
| | | atggattggtgccttcgggaactgtgagacaggtgctgcatggctgtcgtcagctcgtgttgtgaaatgttgggttaagtc |
| | | ccgcaacgagcgcaacccttatcctttgagccagcggtccggccgggaactcaaaggagactgccagtgataaactg |
| | | gaggaaggtggggatgacgtcaagtcatcatggcccttacgaccagggctacacacgtgctacaatggcgcatacaa |
| | | agagaagcgacctcgcgagagcaagcggacctcataaagtgcgtcgtagtccggattggagtctgcaactcgactcc |
| | | atgaagtcggaatcgctagtaatcgtggatcagaatgccacggtgaatacgacccgggccagtacacaccgcccgtc |
| | | acaccatgggagtgggagcaaaagaagtaggtagcttaaccacgggagggcgcttaccactagtgattcatgactgg |
| | | ggtgaagtcgtaacaaggtaaccgtaggggaacctgcggttggatcacctcctt |
| Escherichia_coli | SEQ ID | ttgaagagtttgatcatggctcagattgaacgctggcggcaggcctaacacatgcaagtcgaacggtaacaggaagaa |
| | NO: 393 | gcttgcactttgctgacgagtggcggacgggtgagtaatgtagggaaactgcctgatggagggggataactactgga |
| | | aacggtagctaataccgcataacgtcgcaagaccaaagagggggaccttcgggcctatgccatcggatgtgcccag |
| | | atgggattagctagtaggtggggtaaaggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtatgaagaaggccttcgggttgtaaagtactttcagcggggaggaagggagtaaagttaatac |
| | | ctttgctcattgacgttacccgcagaagaagcaccggctaactccgtgccagcagccgcggtaatacggagggtgcaa |
| | | gcgttaatcggaattactgggcgtaaagcgcacgcaggcggtttgttaagtcagatgtgaaatccccgggctcaacctg |
| | | ggaactgcatctgatactggcaagcttgagtctcgtagaggggggtagaattccaggtgtagcggtgaaatgcgtaga |
| | | gatctggaggaataccggtggcgaaggcggccccctggacgaagactgacgctcaggtgcgaaagcgtggggagc |
| | | aaacaggattagataccctggtagtccacgccgtaaacgatgtcgacttggaggttgtgcccttgaggcgtggcttccg |
| | | gagctaacgcgttaagtcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaaccttgtagag |
| | | atacgagggtgccacgggaactgtgagacaggtgctgcatggctgtcgtcagctcgtgttgtgaaatgttgggttaagt |
| | | cccgcaacgagcgcaacccttatcctagttgccagcggtccggccgggaactcaaaggagactgccagtgataaact |
| | | ggaggaaggtggggatgacgtcaagtcatcatggcccttacgaccagggctacacacgtgctacaatggcgcataca |
| | | aagagaagcgacctcgcgagagcaagcggacctcataaagtgcgtcgtagtccggattggagtctgcaactcgactc |
| | | catgaagtcggaatcgctagtaatcgtggatcagaatgccacggtgaatacgacccgggccagtacacaccgcccgt |
| | | cacaccatgggagtgggttgcaaaagaagtaggtagcttaaccttcgggagggcgataccactagtgattcatgactg |
| | | gggtgaagtcgtaacaaggtaaccgtaggggaacctgcggttggatcacctcctt |
| Escherichia_coli | SEQ ID | ttgaagagtttgatcatggctcagattgaacgctggcggcaggcctaacacatgcaagtcgaacggtaacaggaagaa |
| | NO: 394 | gcttgcttctttgctgacgagtggcggacgggtgagtaatgtctgggaaactgcctgatggagggggataactactgga |
| | | aacggtagctaataccgcataacgtcgcaagaccaaagagggggaccttcgggcctcttgccatcggatgtgcccag |
| | | atgggattagctagtaggtggggtaaaggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtatgaagaaggccttcgggttgtaaagtactttcagcggggaggaagggagtaaagttaatac |
| | | ctttgctcattgacgttacccgcagaagaagcaccggctaactccgtgccagcagccgcggtaatacggagggtgcaa |
| | | gcgttaatcggaattactgggcgtaaagcgcacgcaggcggtagttaagtcagatgtgaaatccccgggctcaacctg |
| | | ggaactgcatctgatactggcaagcttgagtctcgtagaggggggtagaattccaggtgtagcggtgaaatgcgtaga |
| | | gatctggaggaataccggtggcgaaggcggccccctggacgaagactgacgctcaggtgcgaaagcgtggggagc |
| | | aaacaggattagataccctggtagtccacgccgtaaacgatgtcgacttggaggttgtgcccttgaggcgtggcttccg |
| | | gagctaacgcgttaagtcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaaccttgtagag |
| | | atacgagggtgccttcgggaactgtgagacaggtgctgcatggctgtcgtcagctcgtgttgtgaaatgttgggttaagt |
| | | cccgcaacgagcgcaacccttatcctagttgccagcggtccggccgggaactcaaaggagactgccagtgataaact |
| | | ggaggaaggtggggatgacgtcaagtcatcatggccatacgaccagggctacacacgtgctacaatggcgcataca |
| | | aagagaagcgacctcgcgagagcaagcggacctcataaagtgcgtcgtagtccggattggagtctgcaactcgactc |
| | | catgaagtcggaatcgctagtaatcgtggatcagaatgccacggtgaatacgacccgggccagtacacaccgcccgt |
| | | cacaccatgggagtgggttgcaaaagaagtaggtagcttaaccttcgggagggcgcttaccactagtgattcatgactg |
| | | gggtgaagtcgtaacaaggtaaccgtaggggaacctgcggttggatcacctcctt |
| Klebsiella_pneumoniae | SEQ ID | ttgaagagtttgatcatggctcagattgaacgctggcggcaggcctaacacatgcaagtcgagcggtagcacagagag |
| | NO: 395 | cttgctctcgggtgacgagcggcggacgggtgagtaatgtctgggaaactgcctgatggagggggataactactgga |
| | | aacggtagctaataccgcataacgtcgcaagaccaaagtgggggaccttcgggcctcatgccatcagatgtgcccag |
| | | atgggattagctagtaggtggggtaacggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtgtgaagaaggccttcgggttgtaaagcactttcagcggggaggaaggcggtgaggttaataa |
| | | cctcatcgattgacgttacccgcagaagaagcaccggctaactccgtgccagcagccgcggtaatacggagggtgca |
| | | agcgttaatcggaattactgggcgtaaagcgcacgcaggcggtctgtcaagtcggatgtgaaatccccgggcttaacct |
| | | gggaactgcattcgaaactggcaggctagagtcttgtagaggggggtagaattccaggtgtagcggtgaaatgcgtag |
| | | agatctggaggaataccggtggcgaaggcggccccctggacaaagactgacgctcaggtgcgaaagcgtggggag |
| | | caaacaggattagataccctggtagtccacgccgtaaacgatgtcgataggaggttgtgcccttgaggcgtggatccg |
| | | gagctaacgcgttaaatcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaactttccagag |
| | | atggattggtgccttcgggaactgtgagacaggtgctgcatggctgtcgtcagctcgtgttgtgaaatgttgggttaagtc |
| | | ccgcaacgagcgcaacccttatcctagttgccagcggtccggccgggaactcaaaggagactgccagtgataaactg |
| | | gaggaaggtggggatgacgtcaagtcatcatggccatacgaccagggctacacacgtgctacaatggcatatacaaa |
| | | gagaagcgacctcgcgagagcaagcggacctcataaagtatgtcgtagtccggattggagtctgcaactcgactccat |
| | | gaagtcggaatcgctagtaatcgtagatcagaatgctacggtgaatacgttcccgggccttgtacacaccgcccgtcac |
| | | accatgggagtgggagcaaaagaagtaggtagcttaaccttcgggagggcgcttaccactagtgattcatgactggg |
| | | gtgaagtcgtaacaaggtaaccgtaggggaacctgcggttggatcacctcctt |
| Klebsiella_pneumoniae | SEQ ID | ttgaagagtttgatcatggctcagattgaacgctggcggcaggcctaacacatgcaagtcgagcggtagcacagagag |
| | NO: 396 | cttgctctcgggtgacgagcggcggacgggtgagtaatgtctgggaaactgcctgatggagggggataactactgga |
| | | aacggtagctaataccgcataacgtcgcaagaccaaagtgggggaccttcgggcctcatgccatcagatgtgcccag |
| | | atgggattagctagtaggtggggtaacggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtgtgaagaaggccttcgggagtaaagcactacagcggggaggaaggcgttaaggttaataa |
| | | ccttggcgattgacgttacccgcagaagaagcaccggctaactccgtgccagcagccgcggtaatacggagggtgca |
| | | agcgttaatcggaattactgggcgtaaagcgcacgcaggcggtctgtcaagtcggatgtgaaatccccgggctcaacc |
| | | tgggaactgcattcgaaactggcaggctagagtcttgtagaggggggtagaattccaggtgtagcggtgaaatgcgta |
| | | gagatctggaggaataccggtggcgaaggcggccccctggacaaagactgacgctcaggtgcgaaagcgtgggga |
| | | gcaaacaggattagataccctggtagtccacgccgtaaacgatgtcgatttggaggttgtgcccttgaggcgtggatcc |
| | | ggagctaacgcgttaaatcgaccgcctggggagtacggccgcaaggttaaaactcaaatgaattgacgggggcccgc |
| | | acaagcggtggagcatgtggtttaattcgatgcaacgcgaagaaccttacctggtcttgacatccacagaactttccaga |
| | | gatggattggtgccttcgggaactgtgagacaggtgctgcatggctgtcgtcagctcgtgttgtgaaatgttgggttaagt |
| | | cccgcaacgagcgcaacccttatcctagttgccagcggtccggccgggaactcaaaggagactgccagtgataaact |
| | | ggaggaaggtggggatgacgtcaagtcatcatggccatacgaccagggctacacacgtgctacaatggcatatacaa |
| | | agagaagcgacctcgcgagagcaagcggacctcataaagtatgtcgtagtccggattggagtctgcaactcgactcca |
| | | tgaagtcggaatcgctagtaatcgtagatcagaatgctacggtgaatacgttcccgggccttgtacacaccgcccgtca |
| | | caccatgggagtgggagcaaaagaagtaggtagcttaaccacgggagggcgcttaccactagtgattcatgactgg |
| | | ggtgaagtcgtaacaaggtaaccgtaggggaacctgcggttggatcacctcctt |
| Klebsiella_pneumoniae | SEQ ID | ttgaagagtttgatcatggctcagattgaacgctggcggcaggcctaacacatgcaagtcgagcggtagcacagagag |
| | NO: 397 | cttgctctcgggtgacgagcggcggacgggtgagtaatgtctgggaaactgcctgatggagggggataactactgga |
| | | aacggtagctaataccgcataatgtcgcaagaccaaagtgggggaccttcgggcctcatgccatcagatgtgcccaga |
| | | tgggattagctagtaggtggggtaacggctcacctaggcgacgatccctagctggtctgagaggatgaccagccacac |
| | | tggaactgagacacggtccagactcctacgggaggcagcagtggggaatattgcacaatgggcgcaagcctgatgc |
| | | agccatgccgcgtgtgtgaagaaggccttcgggttgtaaagcactttcagcggggaggaaggcggtgaggttaataac |
| | | ctcatcgattgacgttacccgcagaagaagcaccggctaactccgtgccagcagccgcggtaatacggagggtgcaa |
| | | gcgttaatcggaattactgggcgtaaagcgcacgcaggcggtctgtcaagtcggatgtgaaatccccgggctcaacct |
| | | gggaactgcattcgaaactggcaggctagagtcttgtagaggggggtagaattccaggtgtagcggtgaaatgcgtag |
| | | agatctggaggaataccggtggcgaaggcggccccctggacaaagactgacgctcaggtgcgaaagcgtggggag |
| | | caaacaggattagataccctggtagtccacgccgtaaacgatgtcgataggaggttgtgcccttgaggcgtggatccg |
| | | gagctaacgcgttaaatcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaactttccagag |
| | | atggattggtgccttcgggaactgtgagacaggtgctgcatggctgtcgtcagctcgtgttgtgaaatgttgggttaagtc |
| | | ccgcaacgagcgcaacccttatcctagttgccagcggtccggccgggaactcaaaggagactgccagtgataaactg |
| | | gaggaaggtggggatgacgtcaagtcatcatggcccttacgaccagggctacacacgtgctacaatggcatatacaaa |
| | | gagaagcgacctcgcgagagcaagcggacctcataaagtatgtcgtagtccggattggagtctgcaactcgactccat |
| | | gaagtcggaatcgctagtaatcgtagatcagaatgctacggtgaatacgttcccgggccttgtacacaccgcccgtcac |
| | | accatgggagtgggagcaaaagaagtaggtagcttaaccttcgggagggcgcttaccactagtgattcatgactggg |
| | | gtgaagtcgtaacaaggtaaccgtaggggaacctgcggaggatcacctcctt |
| Klebsiella_pneumoniae | SEQ ID | ttgaagagtttgatcatggctcagattgaacgctggcggcaggcctaacacatgcaagtcgagcggtagcacagagag |
| | NO: 398 | cttgctctcgggtgacgagcggcggacgggtgagtaatgtctgggaaactgcctgatggagggggataactactgga |
| | | aacggtagctaataccgcataacgtcgcaagaccaaagtgggggaccttcgggcctcatgccatcagatgtgcccag |
| | | atgggattagctagtaggtggggtaacggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtgtgaagaaggccttcgggagtaaagcactacattggggaggaaggcgttaaggttaataa |
| | | ccttggcgattgacgttacccgcagaagaagcaccggctaactccgtgccagcagccgcggtaatacggagggtgca |
| | | agcgttaatcggaattactgggcgtaaagcgcacgcaggcggtctgtcaagtcggatgtgaaatccccgggctcaacc |
| | | tgggaactgcattcgaaactggcaggctagagtcttgtagaggggggtagaattccaggtgtagcggtgaaatgcgta |
| | | gagatctggaggaataccggtggcgaaggcggccccctggacaaagactgacgctcaggtgcgaaagcgtgggga |
| | | gcaaacaggattagataccctggtagtccacgccgtaaacgatgtcgatttggaggttgtgcccttgaggcgtggcttcc |
| | | ggagctaacgcgttaaatcgaccgcctggggagtacggccgcaaggttaaaactcaaatgaattgacgggggcccgc |
| | | acaagcggtggagcatgtggtttaattcgatgcaacgcgaagaaccttacctggtcttgacatccacagaactttccaga |
| | | gatggattggtgctttcgggaactgtgagacaggtgctgcatggctgtcgtcagctcgtgagtgaaatgttgggttaagt |
| | | cccgcaacgagcgcaacccttatcctttgttgccagcggttcggccgggaactcaaaggagactgccagtgataaact |
| | | ggaggaaggtggggatgacgtcaagtcatcatggcccttacgaccagggctacacacgtgctacaatggcatatacaa |
| | | agagaagcgacctttgcgagagcaagcggacctcataaagtatgtcgtagtttcggattggagtctgcaactcgactcca |
| | | tgaagtcggaatcgctagtaatcgtagatcagaatgctacggtgaatacgttcccgggccttgtacacaccgcccgtca |
| | | caccatgggagtgggttgcaaaagaagtaggtagcttaaccttcgggagggcgcttaccactttgtgattcatgactgg |
| | | ggtgaagtcgtaacaaggtaaccgtaggggaacctgcggttggatcacctcctt |
| Klebsiella_pneumoniae | SEQ ID | ttgaagagtttgatcatggctcagattgaacgctggcggcaggcctaacacatgcaagtcgagcggtagcacagagag |
| | NO: 399 | cttgctctcgggtgacgagcggcggacgggtgagtaatgtctgggaaactgcctgatggagggggataactactgga |
| | | aacggtagctaataccgcataatgtcgcaagaccaaagtgggggaccttcgggcctcatgccatcagatgtgcccaga |
| | | tgggattagctagtaggtggggtaacggctcacctaggcgacgatccctagctggtctgagaggatgaccagccacac |
| | | tggaactgagacacggtccagactcctacgggaggcagcagtggggaatattgcacaatgggcgcaagcctgatgc |
| | | agccatgccgcgtgtgtgaagaaggccttcgggttgtaaagcactttcagcggggaggaaggcggtgaggttaataac |
| | | cttggcgattgacgttacccgcagaagaagcaccggctaactccgtgccagcagccgcggtaatacggagggtgcaa |
| | | gcgttaatcggaattactgggcgtaaagcgcacgcaggcggtctgtcaagtcggatgtgaaatccccgggctcaacct |
| | | gggaactgcattcgaaactggcaggctagagtcttgtagaggggggtagaattccaggtgtagcggtgaaatgcgtag |
| | | agatctggaggaataccggtggcgaaggcggccccctggacaaagactgacgctcaggtgcgaaagcgtggggag |
| | | caaacaggattagataccctggtagtccacgccgtaaacgatgtcgatttggaggttgtgcccttgaggcgtggcttccg |
| | | gagctaacgcgttaaatcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaacttagcaga |
| | | gatgctttggtgccttcgggaactgtgagacaggtgctgcatggctgtcgtcagctcgtgttgtgaaatgttgggttaagt |
| | | cccgcaacgagcgcaacccttatccatgttgccagcggtccggccgggaactcaaaggagactgccagtgataaact |
| | | ggaggaaggtggggatgacgtcaagtcatcatggccatacgaccagggctacacacgtgctacaatggcatatacaa |
| | | agagaagcgacctcgcgagagcaagcggacctcataaagtatgtcgtagtccggattggagtctgcaactcgactcca |
| | | tgaagtcggaatcgctagtaatcgtagatcagaatgctacggtgaatacgttcccgggccttgtacacaccgcccgtca |
| | | caccatgggagtgggttgcaaaagaagtaggtagcttaaccttcgggagggcgcttaccactttgtgattcatgactgg |
| | | ggtgaagtcgtaacaaggtaaccgtaggggaacctgcggttggatcacctcctt |
| Klebsiella_pneumoniae | SEQ ID | ttgaagagatgatcatggctcagattgaacgctggcggcaggcctaacacatgcaagtcgagcggtagcacagagag |
| | NO: 400 | cttgctctcgggtgacgagcggcggacgggtgagtaatgtctgggaaactgcctgatggagggggataactactgga |
| | | aacggtagctaataccgcataacgtcgcaagaccaaagtgggggaccttcgggcctcatgccatcagatgtgcccag |
| | | atgggattagctagtaggtggggtaacggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtgtgaagaaggccttcgggagtaaagcactacagcggggaggaaggcggtgaggttaataa |
| | | cctcatcgattgacgttacccgcagaagaagcaccggctaactccgtgccagcagccgcggtaatacggagggtgca |
| | | agcgttaatcggaattactgggcgtaaagcgcacgcaggcggtctgtcaagtcggatgtgaaatccccgggcttaacct |
| | | gggaactgcattcgaaactggcaggctagagtcttgtagaggggggtagaattccaggtgtagcggtgaaatgcgtag |
| | | agatctggaggaataccggtggcgaaggcggccccctggacaaagactgacgctcaggtgcgaaagcgtggggag |
| | | caaacaggattagataccctggtagtccacgccgtaaacgatgtcgataggaggttgtgcccttgaggcgtggatccg |
| | | gagctaacgcgttaaatcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaactttccagag |
| | | atggattggtgccttcgggaactgtgagacaggtgctgcatggctgtcgtcagctcgtgttgtgaaatgttgggttaagtc |
| | | ccgcaacgagcgcaacccttatcctagttgccagcggtccggccgggaactcaaaggagactgccagtgataaactg |
| | | gaggaaggtggggatgacgtcaagtcatcatggcccttacgaccagggctacacacgtgctacaatggcatatacaaa |
| | | gagaagcgacctcgcgagagcaagcggacctcataaagtatgtcgtagtccggattggagtctgcaactcgactccat |
| | | gaagtcggaatcgctagtaatcgtagatcagaatgctacggtgaatacgttcccgggccttgtacacaccgcccgtcac |
| | | accatgggagtgggagcaaaagaagtaggtagcttaaccttcgggagggcgcttaccactagtgattcatgactggg |
| | | gtgaagtcgtaacaaggtaaccgtaggggaacctgcggttggatcacctcctt |
| Klebsiella_pneumoniae | SEQ ID | ttgaagagtttgatcatggctcagattgaacgctggcggcaggcctaacacatgcaagtcgagcggtagcacagagag |
| | NO: 401 | cttgctctcgggtgacgagcggcggacgggtgagtaatgtctgggaaactgcctgatggagggggataactactgga |
| | | aacggtagctaataccgcataatgtcgcaagaccaaagtgggggaccttcgggcctcatgccatcagatgtgcccaga |
| | | tgggattagcttgtaggtggggtaacggctcacctaggcgacgatccctagctggtctgagaggatgaccagccacac |
| | | tggaactgagacacggtccagactcctacgggaggcagcagtggggaatattgcacaatgggcgcaagcctgatgc |
| | | agccatgccgcgtgtgtgaagaaggccttcgggttgtaaagcactttcagcggggaggaaggcggtgaggttaataac |
| | | ctcatcgattgacgttacccgcagaagaagcaccggctaactccgtgccagcagccgcggtaatacggagggtgcaa |
| | | gcgttaatcggaattactgggcgtaaagcgcacgcaggcggtctgtcaagtcggatgtgaaatccccgggctcaacct |
| | | gggaactgcattcgaaactggcaggctagagtcttgtagaggggggtagaattccaggtgtagcggtgaaatgcgtag |
| | | agatctggaggaataccggtggcgaaggcggccccctggacaaagactgacgctcaggtgcgaaagcgtggggag |
| | | caaacaggattagataccctggtagtccacgccgtaaacgatgtcgataggaggttgtgcccttgaggcgtggatccg |
| | | gagctaacgcgttaaatcgaccgcctggggagtacggccgcaaggttaaaactcaaatgaattgacgggggcccgca |
| | | caagcggtggagcatgtggtttaattcgatgcaacgcgaagaaccttacctggtcttgacatccacagaacttagcaga |
| | | gatgctttggtgccttcgggaactgtgagacaggtgctgcatggctgtcgtcagctcgtgagtgaaatgttgggttaagt |
| | | cccgcaacgagcgcaacccttatcctagttgccagcggtccggccgggaactcaaaggagactgccagtgataaact |
| | | ggaggaaggtggggatgacgtcaagtcatcatggcccttacgaccagggctacacacgtgctacaatggcatatacaa |
| | | agagaagcgacctcgcgagagcaagcggacctcataaagtatgtcgtagtccggattggagtctgcaactcgactcca |
| | | tgaagtcggaatcgctagtaatcgtagatcagaatgctacggtgaatacgttttccgggccagtacacaccgcccgtca |
| | | caccatgggagtgggttgcaaaagaagtaggtagcttaaccttcgggagggcgcttaccactttgtgattcatgactgg |
| | | ggtgaagtcgtaacaaggtaaccgtaggggaacctgcggttggatcacctcctt |
| Klebsiella_pneumoniae | SEQ ID | ttgaagagtttgatcatggctcagattgaacgctggcggcaggcctaacacatgcaagtcgagcggtagcacagagag |
| | NO: 402 | cagctctcgggtgacgagcggcggacgggtgagtaatgtctgggaaactgcctgatggagggggataactactgga |
| | | aacggtagctaataccgcataacgtcgcaagaccaaagtgggggaccttcgggcctcatgccatcagatgtgcccag |
| | | atgggattagctagtaggtggggtaacggctcacctaggcgacgatccctagctggtctgagaggatgaccagccaca |
| | | ctggaactgagacacggtccagactcctacgggaggcagcagtggggaatattgcacaatgggcgcaagcctgatg |
| | | cagccatgccgcgtgtgtgaagaaggccttcgggttgtaaagcactttcagcggggaggaaggcgttaaggttaataa |
| | | cctcatcgattgacgttacccgcagaagaagcaccggctaactccgtgccagcagccgcggtaatacggagggtgca |
| | | agcgttaatcggaattactgggcgtaaagcgcacgcaggcggtctgtcaagtcggatgtgaaatccccgggctcaacc |
| | | tgggaactgcattcgaaactggcaggctagagtcttgtagaggggggtagaattccaggtgtagcggtgaaatgcgta |
| | | gagatctggaggaataccggtggcgaaggcggccccctggacaaagactgacgctcaggtgcgaaagcgtgggga |
| | | gcaaacaggattagataccctggtagtccacgccgtaaacgatgtcgatttggaggttgtgcccttgaggcgtggcttcc |
| | | ggagctaacgcgttaaatcgaccgcctggggagtacggccgcaaggttaaaactcaaatgaattgacgggggcccgc |
| | | acaagcggtggagcatgtggtttaattcgatgcaacgcgaagaaccttacctggtcttgacatccacagaacttagcag |
| | | agatgctttggtgccttcgggaactgtgagacaggtgctgcatggctgtcgtcagctcgtgttgtgaaatgttgggttaag |
| | | tcccgcaacgagcgcaacccttatcctttgttgccagcggtccggccgggaactcaaaggagactgccagtgataaac |
| | | tggaggaaggtggggatgacgtcaagtcatcatggcccttacgaccagggctacacacgtgctacaatggcatataca |
| | | aagagaagcgacctcgcgagagcaagcggacctcataaagtatgtcgtagtccggattggagtctgcaactcgactcc |
| | | atgaagtcggaatcgctagtaatcgtagatcagaatgctacggtgaatacgttcccgggccttgtacacaccgcccgtc |
| | | acaccatgggagtgggttgcaaaagaagtaggtagcttaaccttcgggagggcgcttaccactttgtgattcatgactgg |
| | | ggtgaagtcgtaacaaggtaaccgtaggggaacctgcggttggatcacctcctt |
| Fusobacterium_ | SEQ ID | gctcaccaaggcgatgatgggtagccggcctgagagggtgatcggccacaaggggactgagacacggcccttactc |
| nucleatum | NO: 403 | ctacgggaggcagcagtggggaatattggacaatggaccaagagtctgatccagcaattctgtgtgcacgatgaagttt |
| | | ttcggaatgtaaagtgctttcagttgggaagaaaaaaatgacggtaccaacagaagaagtgacggctaaatacgtgcca |
| | | gcagccgcggtaatacgtatgtcacaagcgttatccggatttattgggcgtaaagcgcgtctaggtggttatgtaagtctg |
| | | atgtgaaaatgcagggctcaactctgtattgcgttggaaactgtgtaactagagtactggagaggtaagcggaactaca |
| | | agtgtagaggtgaaattcgtagatatttgtaggaatgccgatggggaagccagcttactggacagatactgacgctgaa |
| | | gcgcgaaagcgtgggtagcaaacaggattagataccctggtagtccacgccgtaaacgatgattactaggtgttgggg |
| | | gtcgaacctcagcgcccaagcaaacgcgataagtaatccgcctggggagtacgtacgcaagtatgaaactcaaagga |
| | | attgacggggacccgcacaagcggtggagcatgtggtttaattcgacgcaacgcgaggaaccttaccagcgtttgaca |
| | | tcttaggaatgagacagagatgtttcagtgtcccttcggggaaacctaaagacaggtggtgcatggctgtcgtcagctcg |
| | | tgtcgtgagatgttgggttaagtcccgcaacgagcgcaacccctttcgtatgttaccatcattaagttggggactcatgcg |
| | | atactgcctgcgatgagcaggaggaaggtggggatgacgtcaagtcatcatgccccttatacgctgggctacacacgt |
| | | gctacaatgggtagtacagagagtcgcaaagccgtgaggtggagctaatctcagaaaactattcttagttcggattgtac |
| | | tctgcaactcgagtacatgaagttggaatcgctagtaatcgcgaatcagcaatgtcgcggtgaatacgttctcgggtcttg |
| | | tacacaccgcccgtcacaccacgagagttggttgcacctgaagtagcaggcctaaccgtaaggagggatgctccgag |
| | | ggtgtgattagcgattggggtgaagtcgtaacaaggtatccgtacgggaacgtgcggatggatcacctcctt |
| Butyrivibrio_sp. | SEQ ID | ttgagagtttgatcctggctcaggatgaacgctggcggcgtgcttaacacatgcaagtcgaacggacggattggagctt |
| | NO: 404 | gctccatgaagttagtggcggacgggtgagtaacgcgtgggtaacctgccctatgcagggggataacgtttggaaacg |
| | | aacgctaataccgcataacctagttggttcgcatgaaccgactagcaaagattttatcggcataggatggacccgcgttg |
| | | gattagctagttggtgagataacagcccaccaaggcaacgatccatagccggcctgagagggtgaacggccacactg |
| | | ggactgagacacggcccagactcctacgggaggcagcagtggggaatattgcacaatgggcgcaagcctgatgcag |
| | | cgacgccgcgtgaaggatgaaggtcttcggatcgtaaacttctatcagcagggaagaaaccatgacggtacctgacta |
| | | agaagccccggctaactacgtgccagcagccgcggtaatacgtagggggcaagcgttatccggaattactgggtgta |
| | | aagggtgcgtaggcggcgatttaagtcagatgtgaaaactcagggctcaaccttgagactgcatctgaaactgagttgc |
| | | tagagtgcaggagaggaaagcggaattccgagtgtagcggtgaaatgcgtagagattcggaggaacaccagtagcg |
| | | aaggcggctttctggactgtaactgacgctgaggcacgaaagcgtggggagcgaacaggattagataccctggtagt |
| | | ccacgccgtaaacgatgaatactaggtgtcggggcttacgggtctcggtgccgcagctaacgcattaagtattccacct |
| | | gggaagtacgaccgcaaggttgaaactcaaaggaattgacggggacccgcacaagcggtggagcatgtggtttaatt |
| | | cgaagcaacgcgaagaaccttacctaaacttgacatccctttgaccggtccttaatcggatctttactagcttgctagtaca |
| | | aaggtgacaggtggtgcatggagtcgtcagctcgtgtcgtgagatgagggttaagtcccgcaacgagcgcaacccct |
| | | atctttagtagccatcattcagttgggcactctagagagactgccagggataacctggaggaaggtggggatgacgtca |
| | | aatcatcatgccccttatgtttagggctacacacgtgctacaatgggtgctacaaagggaagcaaatccgcgaggccga |
| | | gcaaatctcaaaaaagcactcccagttcggattgtagtctgcaactcgactacatgaagttggaatcgctagtaatcgcg |
| | | aatcagaatgtcgcggtgaatacgttcccgggtcagtacacaccgcccgtcacaccatgggagagggggggcccaa |
| | | cgccagtgacctaacccgcaagggaaggagctgtctaaggcaaaaccaatgactggggtgaagtcgtaacaaggta |
| | | gccgtatcggaaggtgcggctggatcacctccttt |
| Ruminococcus_obeum | SEQ ID | cctacgggaggcagcagtggggaatattgcacaatgggggaaaccctgatgcagcgacgccgcgtgaaggaagaa |
| | NO: 405 | gtatctcggtatgtaaacttctatcagcagggaagatagtgacggtacctgactaagaagccccggctaactacgtgcc |
| | | agcagccgcggtaatacgtagggggcaagcgttatccggatttactgggtgtaaagggagcgtagacggactggcaa |
| | | gtctgatgtgaaaggcgggggctcaacccctggactgcattggaaactgttagtcttgagtgccggagaggtaagcgg |
| | | aattcctagtgtagcggtgaaatgcgtagatattaggaggaacaccagtggcgaaggcggcttactggacggtaactg |
| | | acgttgaggctcgaaagcgtggggagcaaacaggattagataccctggtagtccacgccgtaaacgatgaatactag |
| | | gtgttggggagcaaagctcttcggtgccgccgcaaacgcattaagtattccacctggggagtacgttcgcaagaatgaa |
| | | actcaaaggaattgacggggacccgcacaagcggtggagcatgtggtttaattcgaagcaacgcgaagaaccttacc |
| | | aagtcttgacatccctctgaccgactataaccgagtattccttcgggacagaggagacaggtggtgcatggttgtcgtc |
| | | agctcgtgtcgtgagatgttgggttaagtcccgcaacgagcgcaacccctatccccagtagccagcggttcggccggg |
| | | cactctgaggagactgccagggataacctggaggaaggcggggatgacgtcaaatcatcatgccccttatgatttggg |
| | | ctacacacgtgctacaatggcgtaaacaaagggaagcaagcctgcgaaggtaagcaaatcccaaaaataacgtccca |
| | | gttcggactgcagtctgcaactcgactgcacgaagctggaatcgctagtaatcgcggatcagaatgccgcggtgaata |
| | | cgttcccgggtcttgtacacaccgcccgtcacaccatgggagtcagtaacgcccgaagtcagtgacctaaccgcaagg |
| | | gaggagctgccgaaggcgggaccgatgactggggtgaagtcgtaacaaggtagccgtatcggaaggtgcggctgg |
| | | atcacctccttt |

### Example 3: Human Studies-Ulcerative Colitis

Human subjects having mild-moderate ulcerative colitis received placebo only (Arm A), weekly doses of enriched and purified spores prepared from the stool of healthy, screened humans (Arm B), a short course of vancomycin followed by weekly administration of the spores (Arm D), or a short course of vancomycin followed by daily administration of the spores (Arm C).

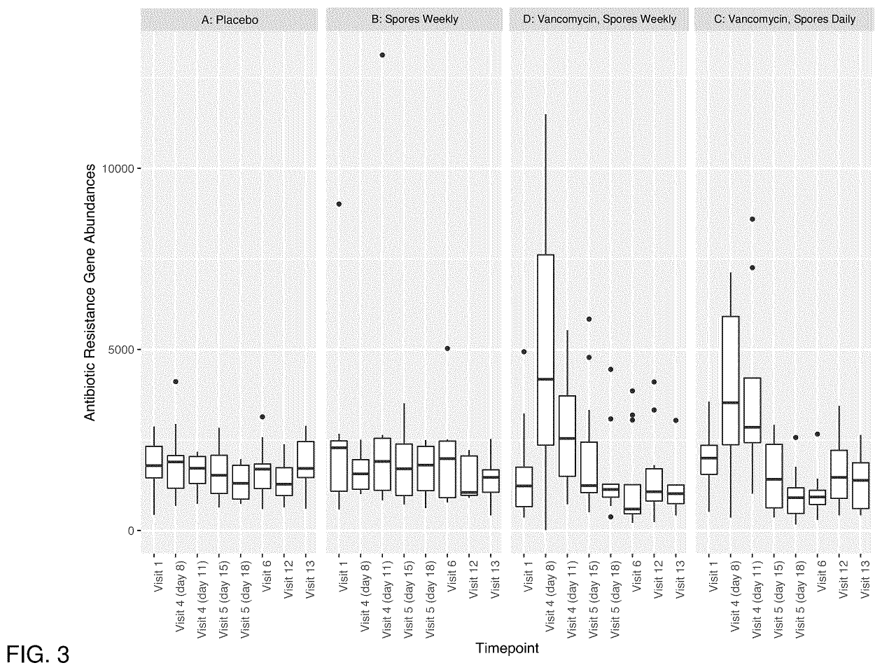

As antibiotics are not the standard of care for ulcerative colitis, this population of subjects is less likely to have been frequently treated with antibiotics as compared to the multiply recurrent C. difficile population (Example 1, above). Therefore, the microbiomes of ulcerative colitis subjects have experienced fewer periods of strong selection for antibiotic resistance. At the pre-antibiotic baseline sample (Visit 1, FIG. 3) we observed a low abundance of antibiotic resistance genes.

The results show that AbxR gene abundances are lower in post-treated time points as compared to post antibiotics baseline samples (Visit 4, day 8). In Arms C and D, treatment with vancomycin results in a spike in antibiotic resistance genes post-treatment (Visit 4, day 8). Treatment with spores daily or weekly resulted in a significant reduction in antibiotic resistance genes at Visit 5 (day 15, 7 days after spore dosing commenced) relative to post-antibiotic baseline (Visit 4, Table 7).

TABLE 7
AbxR gene abundances are lower in select post-treated
time points as compared to post
antibiotics baseline samples (Visit 4, day 8).

| | | Day 11 | Day 15 |
|---|---|---|---|
| | | (3 days post - | (7 days post |
| | | treatment) < Day 8 | treatment) < Day 8 |
| | Arm | (post vancomycin) | (post vancomycin) |
| Wilcox test | D (spores | 0.01367 | 0.03223 |
| p-values | weekly after | | |
| (one sided | vancomycin) | | |
| paired test) | | | |
| Wilcox test | C (spores | 0.6562 | 0.009766 |
| p-value | daily after | | |
| (one sided | vancomycin) | | |
| paired test) | | | |

AbxR gene abundances are lower in post-treated time points as compared to pre-antibiotic baseline samples (Visit 1). In Arms C and D, treatment with spores resulted in reduction in antibiotic resistance genes relative to pre-antibiotic baseline (Visit 1, Table 8, FIG. 3). Treatment with spores daily resulted in a significant reduction in antibiotic resistance genes at Visit 5 and Visit 6 (day 18, day 28, respectively) relative to pre-antibiotic baseline (Visit 1, Table 8).

TABLE 8
Treatment with spores resulted in reduction of AbxR gene
abundance relative to pre-antibiotic baseline
(e.g. Visit l (pre-antibiotic) vs (Visit 5 or Visit 6)).

| | Arms | Visit 5 (day 18) | Visit 6 |
|---|---|---|---|
| Wilcox test p- | D (spores | 0.1826 | 0.1902 |
| values (one sided | weekly after | | |
| paired test) | vancomycin) | | |
| Wilcox test p- | C (spores | 0.01367 | 0.04199 |
| value (one sided | daily after | | |
| paired test) | vancomycin) | | |

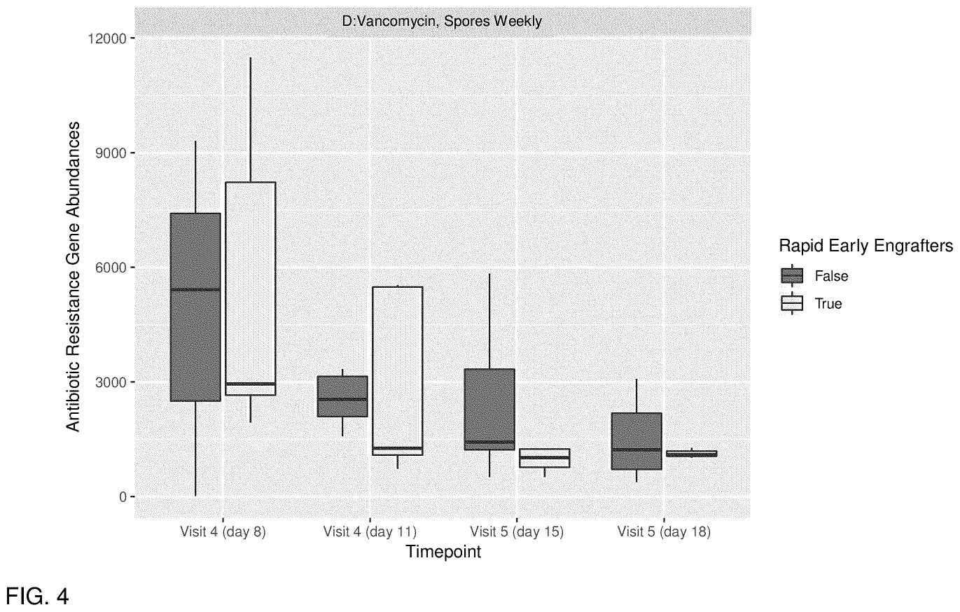

The results show that engraftment of spores drives reduced antibiotic resistance. In Arm D (weekly dosing), spores engrafted rapidly in a subset of subjects (Rapid Early Engrafters=True, FIG. 4). In the remaining subjects, engraftment was reduced in magnitude or timing (Rapid Early Engrafters=False, FIG. 4). “Rapid Early Engrafters” were defined as subjects with more than 6 dosed spore formers detected at visit 5 (Day 15). We observed that these rapid early engrafting subjects displayed significantly lower AbxR gene abundances at Visit 5 (day 15) as compared to those not identified as rapid early engrafters at Visit 5 (day 15, 7 days after first dose of spores).

TABLE 9
Within the same time point AbxR gene abundances are
lower in rapid engrafting subjects as compared
to non-rapid engrafters, Arm D (weekly dosing).

| | | | Day 15 |
|---|---|---|---|
| | Day 8 (pre- | Day 11 (3 days | (6 days post |
| | treatment) | post-treatment) | treatment) |
| Wilcox test p- | 0.6548 | 0.3312 | 0.07343 |
| value | | | |
| (alternative = | | | |
| “less”) | | | |

### Example 4: Animal Models

One method of testing a ROAR composition for the ability to reduce antibiotic resistance uses conventional mice (laboratory strains that have a microbiome). The mice are treated with selected antibiotics, then colonized with bacteria harboring one or more antibiotic resistance genes, for example, vancomycin resistant bacteria (vancomycin resistant Enterococcus; VRE) or vancomycin and carbapenem resistant bacteria (carbapenem resistant Enterobacteriaceae; CRE). Following colonization, the mice are treated with a ROAR composition, fecal transplant, or control. Titers of antibiotic resistant bacteria (for example, vancomycin resistant and carbapenem resistant bacteria) are monitored via plating of stool collected from treated animals.

Applicants have established a reproducible and robust vancomycin-resistant enterococci (VRE) colonization model in mice based on a published model (Caballero and Pamer, 2015, Ann Rev Immunol 33:227-256). Prior to VRE inoculation, specific pathogen free (SPF) mice are pre-conditioned with ampicillin to disrupt colonization resistance and allow VRE colonization. Following ampicillin treatment, mice are challenged with VRE and achieve high titer (107 to 1010 CFU/g feces) carriage in the GI tract, a condition that is stable for multiple weeks. Once colonized with VRE, mice are treated with a human FMT or a ROAR resistance composition via daily oral gavage for 3 days. Fresh fecal samples are collected throughout the study to evaluate the ability of compositions to clear VRE from the mice and restore a healthy gut microbiome. VRE viable titers in feces pre- and post-treatment are determined by plating on selective media that contains bile esculin azide agar with vancomycin and streptomycin. The VRE colonization model is able to detect a >4 log difference in colonization over 3 weeks in mice treated with human FMT compared to mice treated only with PBS. The ability to detect a >4 log difference in VRE titers during the study window has also distinguished designed microbial compositions with differential potency. In addition, the abundance of antibiotic resistance genes can be assayed. Carbapenem-resistant Enterobacteriaceae (CRE) resistance can be tested in a similar way, substituting selective media appropriate for CRE.

Using such models, a ROAR composition can be tested for its effects on the abundance of antibiotic resistance genes. A ROAR composition that can reduce the abundance of one or more antibiotic resistance genes in this model is useful for the methods described herein.

Various embodiments of the invention are within the following numbered paragraphs.

1. A method of reducing the abundance of at least one antibiotic-resistance gene in the microbiome of a subject, the method comprising administering a therapeutically effective amount of a microbiome composition comprising spore-former bacteria.

2. A method of treating a subject at risk for or diagnosed with an undesirable level or population of drug-resistant bacteria, the method comprising administering a therapeutically effective amount of a microbiome composition comprising Firmicutes derived from one or more healthy human subjects.

3. A method of reducing the abundance of antibiotic-resistant bacteria in the microbiome of a subject, the method comprising administering a therapeutically effective amount of a ROAR composition.

4. A method of disrupting a transmission cycle of antibiotic resistance genes, the method comprising administering a ROAR composition to at least two individuals with a high likelihood of direct or indirect contact including contact with a bodily fluid or waste.

5. The method of paragraph 1, wherein the antibiotic-resistance gene(s) is selected from those listed in Table 1.

6. The method of paragraph 5, wherein the abundance of at least 2, 3, 4, 5, 6, 7, 8, 9, 10, or more antibiotic resistance genes of Table 1 is reduced.

7. The method of any one of paragraphs 1 to 6, wherein the drug or antibiotic is from a drug class listed in Table 2.

8. The method of any one of paragraphs 1 to 7, further comprising reducing the abundance of 1, 2, 3, 4, 5, 6, 7, 8, 9, 10, 11, 12, 13, 14, 15, 16, 17, 18, 19, 20, 21, 22, 23, 24, 25, or more antibiotic resistant genes or drug classes.

9. The method of any one of paragraphs 1 to 8, wherein the administered composition comprises one or more species listed in Table 3.

10. The method of paragraph 9, wherein the administered composition comprises 2, 3, 4, 5, 6, 7, 8, 9, 10, 11, 12, 13, 14, 15, 16, 17, 18, 19, 20, or more species listed in Table 3.

11. The method of paragraph 9 or 10, wherein the administered species comprises a 16S rDNA, V4, and/or V6 sequence listed in Table 5, or a sequence having at least 95% identity thereto.

12. The method of any one of paragraphs 1 to 11, wherein the administered composition does not comprise one or more species of Table 4.

13. The method of any one of paragraphs 1 to 12, wherein the bacteria in the composition are in the form of spores.

14. The method of any one of paragraphs 1 to 13, wherein the bacteria in the composition are cultured.

15. The method of any one of paragraphs 1 to 14, wherein the composition comprises Firmicutes and one or more species of Bacteroides.

16. The method of any one of paragraphs 1 to 15, wherein the subject has a reduced abundance of one, two, or three bacteria selected from the group consisting of quinolone resistant, beta-lactam-resistant, and carbapenem-resistant bacteria.

17. The method of any one of paragraphs 1 to 16, wherein the subject has been unresponsive to antibiotic treatment.

18. The method of any one of paragraphs 1 to 17, wherein the subject has or is at risk of developing C. difficile infection.

19. The method of any one of paragraphs 1 to 18, wherein the subject has or is at risk of developing a colitis.

20. The method of paragraph 19, wherein the colitis is Crohn's disease or ulcerative colitis.

21. The method of paragraph 20, wherein the colitis is mild to moderate ulcerative colitis.

22. A composition comprising bacterial species associated with a decrease in the abundance of at least one antibiotic resistance gene in a subject.

23. The composition of paragraph 22, wherein the antibiotic-resistance gene(s) is selected from those listed in Table 1.

24. The composition of paragraph 23, wherein the abundance of at least 2, 3, 4, 5, 6, 7, 8, 9, 10, or more antibiotic resistance genes of Table 1 is decreased.

25. The composition of any one of paragraphs 22 to 24, wherein the antibiotic is from a class listed in Table 2.

26. The composition of any one of paragraphs 22 to 25, wherein the composition comprises one or more species listed in Table 3.

27. The composition of paragraph 26, wherein the composition comprises 2, 3, 4, 5, 6, 7, 8, 9, 10, 11, 12, 13, 14, 15, 16, 17, 18, 19, 20, or more species listed in Table 3.

28. The composition of paragraph 20 or 27, wherein the species comprises a 16S rDNA, V4, and/or V6 sequence listed in Table 5, or a sequence having at least 95% identity thereto.

29. The composition of any one of paragraphs 22 to 28, wherein the composition does not comprise one or more species of Table 4, and optionally the one or more species of Table 4 comprises a 16S rDNA, V4, and/or V6 sequence listed in Table 6, or a sequence having at least 95% identity thereto.

30. The composition of any one of paragraphs 22 to 29, wherein the composition comprises one or more species of Firmicutes.

31. The composition of any one of paragraphs 22 to 30, wherein the composition comprises one or more species of Bacteroides.

32. The composition of any one of paragraphs 22 to 31, wherein the bacteria in the composition are in the form of spores.

33. The composition of any one of paragraphs 22 to 32, wherein the bacteria in the composition are obtained from a spore fraction derived from feces of a healthy subject.

34. The composition of any one of paragraphs 22 to 33, wherein the composition comprises one or more species of Firmicutes, and optionally one or more species of Bacteroides, as the sole bacterial species in the composition.

35. The composition of any one of paragraphs 22 to 34, wherein the bacteria in the composition are cultured.

36. A composition comprising bacteria for use in decreasing the abundance of at least one antibiotic resistance gene in a subject to whom the composition is administered.

37. The composition of paragraph 36, wherein the bacteria in the composition are obtained from a spore fraction derived from feces of a healthy subject.

38. The composition of paragraph 36, wherein the bacteria are selected for being associated with a decrease in the abundance of one or more antibiotic resistance genes in a subject.

39. The composition of any one of paragraphs 36 to 38, wherein the antibiotic resistance gene(s) is selected from Table 1.

40. The composition of any one of paragraphs 36 to 39, wherein the composition comprises at least one species selected from Table 3.

41. The composition of any one of paragraphs 36 to 40, wherein the composition does not comprise a species of Table 4.

42. A method for identifying a bacterial composition useful for decreasing the abundance of at least one antibiotic resistance gene in a subject to whom the composition is administered, the method comprising screening the composition for the presence of one or more bacterial species of Table 3, wherein detection of one or more species of Table 3 in the bacterial composition indicates the identification of a composition that can be used to decrease the abundance of at least one antibiotic resistance gene.

43. The method of paragraph 42, wherein the screening comprises detection of one or more 16S rDNA, V4, and/or V6 sequences of Table 5 in the composition, or a sequence having at least 95% identity thereto.

44. The method of paragraph 42 or 43, further comprising screening for the presence of one or more bacterial species of Table 4, and optionally the one or more species of Table 4 comprises a 16S rDNA, V4, and/or V6 sequence listed in Table 6, or a sequence having at least 95% identity thereto.

45. A method for screening a potential donor of feces for use in therapeutic methods, the method comprising testing a feces sample from the potential donor for the presence of a bacterial species from Table 3 or Table 4, wherein detection of one or more species from Table 3 indicates that the potential donor may proceed to become a donor, while detection of one or more species from Table 4 indicates that the potential donor should not become a donor.

46. A method for determining whether a subject could benefit from treatment with a ROAR composition, the method comprising determining whether a sample from the subject comprises one or more species of bacteria from Table 4, wherein detection of one or more species from Table 4 indicates that the subject may benefit from treatment with a ROAR composition.

47. A composition of any one of paragraphs 22 to 41, wherein the bacteria in the composition demonstrate the ability to decrease the abundance of one or more antibiotic resistance gene(s) in an animal model of antibiotic resistance.

48. The composition of paragraph 47, wherein the animal model comprises a mouse treated with one or more antibiotics and then colonized with one or more bacteria harboring one or more antibiotic resistance genes.

49. The composition of paragraph 48, wherein the mice are colonized with vancomycin resistant bacteria or vancomycin and carbapenem resistant bacteria.

Other embodiments are within the scope of the following claims

1. A method of (a) reducing an abundance of at least one gene that confers resistance to an antibiotic (“antibiotic-resistance gene”) in the microbiome of a subject in need thereof or (b) treating a subject at risk for or diagnosed with an undesirable level or population of bacteria that are resistant to an antibiotic, comprising administering to the subject a therapeutically effective amount of a composition comprising one or more bacteria, wherein the one or more bacteria are selected from Table 3.

2-4. (canceled)

5. The method of claim 1, wherein the at least one antibiotic-resistance gene is selected from those listed in Table 1.

6. The method of claim 5, wherein the abundance of at least 2, 3, 4, 5, 6, 7, 8, 9, 10, or more antibiotic-resistance genes are reduced after the administration.

7. The method of claim 1, wherein the antibiotic belongs to a drug class listed in Table 2.

8-9. (canceled)

10. The method of claim 1, wherein the composition comprises 2, 3, 4, 5, 6, 7, 8, 9, 10, 11, 12, 13, 14, 15, 16, 17, 18, 19, 20, or more bacteria selected from Table 3.

11. The method of claim 1, wherein the one or more bacteria comprise a 16S rDNA sequence that is at least 95% identical to a 16S rDNA sequence set forth in Table 5.

12. The method of claim 1, wherein the composition does not comprise one or more bacteria selected from Table 4.

13. The method of claim 1, wherein the one or more bacteria are in the form of spores, cultured, or both.

14-15. (canceled)

16. The method of claim 1, wherein, prior to the administration, the subject (i) has a reduced abundance of one, two, or three bacteria selected from the group consisting of quinolone resistant, beta-lactam-resistant, and carbapenem-resistant bacteria; (ii) has been unresponsive to an antibiotic treatment; (iii) has or is at risk of developing a Clostridium difficile infection; (iv) has or is at risk of developing a colitis; or (v) any combination of (i) to (iv).

17-21. (canceled)

22. A composition comprising one or more bacteria that are capable of decreasing the abundance of at least one gene that confers resistance to an antibiotic (“antibiotic-resistance gene”) in a subject.

23. The composition of claim 22, wherein the at least one antibiotic-resistance gene is selected from those listed in Table 1.

24-25. (canceled)

26. The composition of claim 22, wherein the one or more bacteria are selected from Table 3.

27-44. (canceled)

45. A method for screening a feces of a potential donor for use in a therapy, the method comprising testing a feces sample from the potential donor for the presence of one or more bacterial species from Table 3 or Table 4, wherein the presence of the one or more bacterial species from Table 3 indicates that the potential donor is a suitable donor for the therapy, while the presence of the one or more bacterial species from Table 4 indicates that the potential donor is not a suitable donor for the therapy.

46-49. (canceled)

50. A method of reducing a resistance of a subject to one or more classes of antibiotics, comprising administering to the subject a therapeutically effective amount of the composition of claim 22.

51. The method of claim 50, wherein the one or more classes of antibiotics are selected from drug classes listed in Table 2.

52. The method of claim 50, wherein after the administration, the resistance of the subject to 2, 3, 4, 5, 6, 7, 8, 9, 10, 11, 12, 13, 14, 15, 16, 17, 18, 19, 20, 21, 22, 23, or 24 different classes of antibiotics is reduced.

53. The method of claim 2, wherein the composition comprises 2, 3, 4, 5, 6, 7, 8, 9, 10, 11, 12, 13, 14, 15, 16, 17, 18, 19, 20, or more bacteria selected from Table 3; or wherein the composition does not comprise one or more bacteria selected from Table 4.

54. The method of claim 2, wherein the one or more bacteria comprise a 16S rDNA sequence that is at least 95% identical to a 16S rDNA sequence set forth in Table 5.

55. A method of producing the composition of claim 22, comprising culturing one or more bacteria selected from Table 3 and combining the one or more bacteria after the culturing.
